(19) United States
(12) Patent Application Publication
(10) Pub. No.: US 2014/0206748 A1
(43) Pub. Date: Jul. 24, 2014

(54) COMPOSITIONS AND METHODS RELATED TO PROSTATE CANCER

(71) Applicant: Regents of the University of Minnesota, Minneapolis, MN (US)

(72) Inventors: Scott Michael Dehm, Plymouth, MN (US); Kevin Andrew Tang Silverstein, Saint Paul, MN (US); Kenneth Bruce Beckman, Saint Paul, MN (US)

(73) Assignee: Regents of the University of Minnesota, Minneapolis, MN (US)

(21) Appl. No.: 14/159,998

(22) Filed: Jan. 21, 2014

Related U.S. Application Data
(60) Provisional application No. 61/754,824, filed on Jan. 21, 2013.

(51) Int. Cl. C12Q 1/68; C07K 14/705

(52) U.S. Cl. CPC C12Q 1/6886; C07K 14/70567
USPC 514/44 A; 435/6.11

(57) ABSTRACT

This disclosure describes markers that can identify patients at risk of developing castration-resistant prostate cancer. The markers, and analyses that use the markers, can be used by health professionals to guide treatment decisions by, for example, helping to evaluate the likelihood that a patient will respond to or develop resistance to prostate cancer therapies targeted to the androgen receptor. Thus, in some cases, methods described herein may be used to identify subjects under treatment for prostate cancer as at risk for developing castration-resistant prostate cancer. Such an evaluation may indicate that a change in prescribed therapy is appropriate. In some of these instances, the change may involve modifying the subject's treatment regimen to include administration of a pharmaceutical composition effective for treating castration-resistant prostate cancer before resistance to androgen receptor-based treatments develops.

### CROSS-REFERENCE TO RELATED APPLICATION

This application claims priority to U.S. Provisional Patent Application Ser. No. 61/754,824, filed Jan. 21, 2013, which is incorporated herein by reference.

### GOVERNMENT FUNDING

This invention was made with government support under W81XWH-10-1-0353, awarded by the Department of Defense. The government has certain rights in the invention.

### SUMMARY

This disclosure describes, in one aspect, a method for detecting expression of an androgen receptor (AR) variant. Generally, the method includes receiving a biological sample obtained from a subject, the biological sample comprising cells expressing a plurality of non-wild-type androgen receptor polynucleotides, each non-wild-type androgen receptor polynucleotide being encoded by a genomic polynucleotide comprising a copy number; performing an assay to measure the copy number of at least one genomic polynucleotide that, when transcribed, produces a non-wild-type androgen receptor polynucleotide; and identifying the sample as exhibiting expression of an AR variant if the at least one genomic polynucleotide exhibits a copy number that differs from the mean AR copy number by at least one standard deviation.

In some embodiments, the non-wild-type androgen receptor polynucleotide can include at least a portion of AR intron 1. In some cases, at least a portion of AR intron 1 can exhibit a copy number that is greater than the mean AR copy number by at least one standard deviation. In other cases, at least a portion of AR intron 1 exhibits a copy number that is less than the mean AR copy number by at least one standard deviation. In one particular embodiment, the non-wild-type androgen receptor polynucleotide can exhibit a 48,476 bp deletion from AR intron 1.

In some embodiments, the non-wild-type androgen receptor polynucleotide can exhibit a deletion of at least a portion of AR exon 5, AR exon 6, or AR exon 7. In certain embodiments, the non-wild-type androgen receptor polynucleotide exhibits an 8579 bp deletion of AR exon 5, AR exon 6, and a portion of AR exon 7.

In some embodiments, the method can further include identifying the subject as at risk for castration-resistant prostate cancer. In some cases, the sample is obtained from a subject that has received treatment for prostate cancer.

In some embodiments, the method can further include either initiating or modifying treatment of the subject based on detecting unbalanced amplification of a polynucleotide that encodes an androgen receptor. In particular embodiment, the method can include administering to the subject at least one pharmaceutical composition effective for treating castration-resistant prostate cancer.

In another aspect, this disclosure describes a method that generally includes administering to a subject at risk of developing castration-resistant prostate cancer a composition that includes an inhibitor of an androgen receptor (AR) splice variant associated with castration-resistant prostate cancer. In some embodiments, such an inhibitor can include a polynucleotide that hybridizes to at least a portion of a transcript of the AR splice variant. In some embodiments, such a polynucleaotide can include a siRNA. In particular embodiments, the AR splice variant can include at least a portion of AR exon 1 or at least a portion of AR exon 7. In other embodiments, the splice variant can include AR 1/2/2b, AR 1/2/3/2b, AR 1/2/3/CE1, AR 1/2/3/CE2, AR 1/2/3/CE3, or ARv567es.

In yet another aspect, this disclosure describes a method for detecting expression of an androgen receptor (AR) variant. Generally, the method includes receiving a biological sample obtained from a subject, the biological sample including at least one cell that expresses an AR variant that includes a linear rearrangement of AR genomic DNA; sequencing a sufficient portion of the AR genomic DNA to detect the linear rearrangement of the AR genomic DNA; and detecting the linear rearrangement of AR genomic DNA.

In some embodiments, the linear rearrangement of the AR genomic DNA can include a deletion of at least a portion of intron 1. In other embodiments, the linear rearrangement of the AR genomic DNA can include a deletion of at least a portion of intron 5, intron 6, and/or intron 7. In still other embodiments, the linear rearrangement of the AR genomic DNA can include an inversion of at least a portion of intron 5, intron 6, and/or intron 7.

In some embodiments, sequencing the portion of the AR genomic DNA can include fragmenting the subject's genomic DNA; hybridizing at least a portion of the fragmented genomic DNA to a polynucleotide complementary to at least a portion of the AR genomic DNA; separating hybridized genomic DNA from non-hybridized genomic DNA; amplifying the hybridized genomic DNA; and sequencing the amplified genomic DNA.

In some embodiments, the method can further include identifying the subject as at risk for castration-resistant prostate cancer. In some embodiments, the method may be performed on a sample obtained from a subject who has received treatment for prostate cancer. In some embodiments, the method further includes either initiating or modifying treatment of the subject based on detecting unbalanced amplification of a polynucleotide that encodes an androgen receptor. In some of these embodiments, the method can include administering to the subject at least one pharmaceutical composition effective for treating castration-resistant prostate cancer.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Diverse and complex patterns of AR gene copy number imbalance in casteate-resistant prostate cancer (CRPCa). (a) Schematic of the AR gene with relative locations of multiplex ligation-dependent probe assay (MLPA) probes used for targeted copy number analysis. Genomic DNA from (b) androgen-dependent (AD) and castration-resistant (CR) PCa cell lines, (c) PCa xenografts, including AD/CR pairs propagated in intact/castrated male mice, and (d) clinical PCa was subjected to MLPA to evaluate genomic copy number across the AR locus and X chromosome.

FIG. 2. Intragenic deletion encompassing AR exons 5, 6, and 7 in the LuCaP 86.2 xenograft model. (a) Relative positions of deletion-spanning PCR primers and expected PCR fragment sizes based on the hg19 build of the human genome. (b) CWR-R1 and BPH-1 genomic DNA was subjected to nested PCR using primer sets indicated in (a). (c) PCR products from (b) were cloned and sequenced using the Sanger method. The electropherogram peak trace and AR gene structure (SEQ ID NO:1433) resulting from the 8,579 bp intragenic deletion are shown. (d) Alignment of the 5′ deletion breakpoint (SEQ ID NO:1432), the 3′ deletion breakpoint (SEQ ID NO:1434), and the deletion fusion (SEQ ID NO:1433) revealed 4 bp of perfect microhomology (solid box) and 10 bp of extended microhomology with 1 bp mismatch (dashed box). Sequence retained in the break fusion junction is shaded in gray.

FIG. 3. Stable, high-level expression of truncated AR variants in CWR-R1 cells. (a) AR genomic organization and exon composition of alternatively spliced AR mRNA isoforms reported in cell lines derived from the CWR22 xenograft. (b) RNA from indicated PCa cells lines was subjected to quantitative RT-PCR with isoform-specific primer sets and Ct values were converted to copy number by plotting on standard curves. (c) CWR-R1 cells were electroporated with a control siRNA or siRNAs targeted to AR exon 1 or exon 7 and analyzed by Western blot with the indicated antibodies 48 hours and 72 hours post-transfection. (d) CWR-R1 cells were treated with 1 nM dihydrotestosterone (DHT) or vehicle (EtOH) for indicated times and analyzed by Western blot with indicated antibodies.

FIG. 4. CWR-R1 cells harbor an intragenic deletion in AR intron 1. A schematic of the AR locus is illustrated at the top. Paired-end sequence reads were mapped to the hg19 build of the human genome using Burrows-Wheeler Alignment (BWA) and visualized using Integrated Genomics Viewer (IGV). Two 50 kb windows spanning genomic positions 66,790,000 and 66,890,000 are shown for each cell line, indicating positions and depth of coverage of BWA-mapped reads. Discordantly-mapped paired-end reads identified by the Hydra workflow are shown for CWR-R1 and the relative location of this predicted deletion is indicated on the AR locus schematic. Differences in coverage peak maxima at sites flanking the predicted deletion breakpoints were compared using t-tests. Positions of individual SureSelect baits used for AR sequence capture are tiled across the bottom. Two large gaps of extended repetitive sequence precluding design of capture baits are indicated.

FIG. 5. Intragenic deletion involving 48,476 bp of AR intron 1 in CWR-R1 cells. (a) Schematic of the AR locus with relative positions of deletion-spanning PCR primers. (b) CWR-R1 genomic DNA was subjected to nested PCR using indicated primer sets indicated in (a). (c) Schematic of the CWR-R1 AR locus harboring a 48,476 bp intron 1 deletion. (d) PCR products from (b) were cloned and sequenced (SEQ ID NO:1436) using the Sanger method. The electropherogram peak trace is shown. (e) Alignment of the 5′ deletion breakpoint (SEQ ID NO:1435), the 3′ deletion breakpoint (SEQ ID NO:1437), and the deletion fusion (residues 1-69 of SEQ ID NO:1436) revealed 3 bp of microhomology (boxed). Sequence retained in the break fusion junction is shaded in gray.

FIG. 6. Intragenic deletion is restricted to a CWR-R1 subpopulation. (a) Schematic of the AR gene with relative locations of primers and probes used for targeted genomic assays. (b) Genomic DNA was subjected to multiplex ligation-dependent probe assay (MLPA) to evaluate genomic copy number across the AR locus and X chromosome. Relative positions of AR-specific MLPA probes are indicated in (a). Gray boxes represent the mean+/−standard deviation of all AR locus probes from 2 independent experiments. Probe pairs displaying copy number greater than 1 standard deviation away from the mean AR copy number are diagnostic of duplication or deletion. Differences in copy number measured by probe pairs flanking predicted duplication or deletion breakpoints were compared with t-tests. (c) Genomic DNA was subjected to nested PCR using primer sets depicted in (a).

FIG. 7. CWR-R1 cells with intron 1 deletion are castration-resistant and dependent on truncated AR variant function. (a) Genomic DNA from early-passage CWR-R1 cells and CWR-R1 cells cultured for 20 passages under castrate conditions was subjected to multiplex ligation-dependent probe assay (MLPA) to evaluate genomic copy number across the AR locus and X chromosome. Gray boxes represent the mean+/−standard deviation of all AR locus probes from 2 independent experiments. Probe pairs diagnostic of deletion are indicated. (b) Lysates from early-passage CWR-R1 cells cultured in the presence (FBS) or absence (CSS) of androgens for 24 hours and CWR-R1 cells cultured in the presence (FBS) or absence (CSS) of androgens for 20 passages (late) were analyzed by Western blot with indicated antibodies. (c) Early-passage CWR-R1 cells and CWR-R1 cells cultured for 20 passages under castrate conditions (late) were transfected with MMTV-Luc, non-targeted control (CTRL) siRNA, or siRNAs targeted to AR exon 1 or exon 7. Cells were grown 24 hours in serum-free medium and treated with 1 nM mibolerone (synthetic androgen) or EtOH (vehicle control) for 24 hours. Luciferase activity was determined. Data represent the mean+/−S.E. from two independent experiments, each performed in duplicate. MMTV promoter activity without androgens and siRNAs was arbitrarily set to 1. (d) Early-passage CWR-R1 cells and CWR-R1 cells cultured for 20 passages under castrate conditions (late) were transfected with non-targeted control (CTRL) siRNA, or siRNAs targeted to AR exon 1 or AR exon 7. Growth of transfected cells under castrate conditions was monitored every two days.

FIG. 8. Agilent SureSelect bait library for AR sequence capture. Individual 120 bp RNA baits were visualized by tiling against human genome build 19 (hg19) using the UCSC genome browser. Repetitive genomic DNA elements identified by RepeatMasker (Tarailo-Graovac and Chen, “Using RepeatMasker to identify repetitive elements in genomic sequences,” Curr Protoc Bioinformatics 2009; Chapter 4: Unit 4.10) are indicated. Three extended regions of repetitive DNA precluded the design of capture baits, resulting in three large gaps (indicated) in the SureSelect bait library.

FIG. 9. Increased copy number within a duplicated segment in 22Rv1 cells. A schematic of the AR locus is illustrated at the top. Paired-end sequence reads were mapped to the hg19 build of the human genome using the Burrows-Wheeler Alignment tool (BWA) and visualized using Integrated Genomics Viewer (IGV). Two overlapping 50 kb windows spanning genomic positions 66,870,000-66,950,000 are shown for each cell line, indicating positions and depth of coverage of BWA-mapped reads. Differences in coverage peak maxima at sites flanking the known duplication breakpoints were compared using t-tests. Positions of individual SureSelect baits used for AR sequence capture are tiled across the bottom. Two large gaps of extended repetitive sequence precluding design of capture baits are indicated.

FIG. 10. Increased copy number of the AR exon 3 segment identified by next-generation re-sequencing of the AR gene in 22Rv1 cells. Top: schematic of the AR gene with locations of the 22Rv1 tandem duplication breakpoints identified in Li et al. (2011 Cancer Res 71:2108-17). Bar graphs represent mean+/−S.D. of 10 consecutive coverage peak maxima located before and after the indicated breakpoints.

FIG. 11. Decreased copy number of the AR intron 1 segment identified by next-generation re-sequencing of the AR gene in CWR-R1 cells. Top: schematic of the AR gene with locations of the predicted CWR-R1 deletion breakpoints. Bar graphs represent mean+/−S.D. of 10 consecutive coverage peak maxima located before and after the indicated breakpoints.

FIG. 12. Illumina reads spanning the CWR-R1 break-fusion junction. Top: schematic of the shorter AR locus in CWR-R1 cells harboring a 48,476 bp intragenic deletion in AR intron 1. Singleton 76 bp Illumina reads were aligned to a 130 bp sequence (SEQ ID NO:1438) harboring the CWR-R1 deletion break fusion using BWA. Aligned reads and coverage plots were visualized using the Integrated Genomics Viewer (IGV).

FIG. 13. Enrichment for cells harboring the AR intron 1 break fusion junction signature in CWR-R1 late cells. Genomic DNA from CWR-R1 early and CWR-R1 late cells was subjected to nested PCR using indicated primer pairs.

FIG. 14. Increased splicing of AR exon CE3 in CWR-R1 late vs. CWR-R1 early cells is stable and not an acute cellular response to hormonal manipulations. (A) RNA from CWR-R1 early and CWR-R1 late cells was subjected to quantitative RT-PCR with isoform-specific primer sets and Ct values were converted to copy number by plotting on standard curves. (B) CWR-R1 early and CWR-R1 late cells were cultured in the presence (DHT) and absence (EtOH) of androgens as indicated and subjected to quantitative RT-PCR with isoform-specific primer sets. Fold changes in expression were determined relative to a GAPDH internal standard using the formula 2^{-ΔΔCt}. (C) CWR-R1 early and CWR-R1 late cells were cultured in the presence (DHT) and absence (EtOH) of androgens as indicated and subjected to Western blot with indicated antibodies.

FIG. 15. Increased nuclear expression of the AR amino-terminal domain in CWR-R1 late cells. (A) Locations of AR epitopes recognized by polyclonal antibodies. (B) CWR-R1 early and CWR-R1 late cells were cultured 72 hours under androgen-free conditions and subjected to immunohistochemical staining using antibodies indicated in (A). Black bars at bottom right of each panel indicates 100 μm.

FIG. 16. AR variants (AR-Vs) support resistance to full-length AR targeting in 22Rv1 cells. (A) 22Rv1 cells were cultured under castrate (CSS) conditions with 10 μM bicalutamide (bic) or 1 μM enzalutamide (enz). (B) MMTV promoter activities in siRNA-transfected cells treated under castrate conditions with 1 nM DHT, 10 μM bicalutamide, or 1 μM enzalutamide. Data represent mean+/−S.E. from at least three independent experiments, each performed in duplicate. Inset: Western blot with antibodies targeted to the AR NTD or an internal control (ERK-2). Locations of full-length AR and truncated AR-Vs are indicated. (C) 22Rv1 cells were transfected with siRNAs under castrate conditions. Gene expression was assessed by quantitative RT-PCR. Bars represent mean+/−S.D. from two biological replicates, each performed in duplicate. Western blots were performed as in (C). (D) 22Rv1 cells were transfected and treated as in (B). Growth was assessed at indicated time-points. Data represent mean+/−S.D. from a quadruplicate experiment representative of two biological replicates. Inset: Western blots were performed as in (B).

FIG. 17. Rearrangement-positive CWR-R1 cells are resistant to full-length AR targeting. (A) Schematic of the AR locus with location of PCR primers for deletion analysis. (B) CWR-R1 single-cell clones were assessed for AR-V expression by Western blot using an antibody specific for the AR NTD. Concurrently, genomic DNA was isolated and subjected to deletion-specific PCR. (C) Deletion-negative clones were cultured under castrate conditions with 1 nM DHT, 10 μM bicalutamide, or 1 μM enzalutamide. Growth was assessed at indicated time-points. Data represent mean+/−S.D. from a quadruplicate experiment representative of two biological replicates. (D) Deletion-positive clones were cultured, treated, and subjected to growth assays as in (C). Deletion-positive clones were further transfected with siRNAs and subjected to growth assays at days 0, 4, and 6. Data represent mean+/−S.D. from a quadruplicate experiment representative of two biological replicates.

FIG. 18. AR-Vs support the androgen/AR transcriptional program. (A) CWR-R1 cells transfected with siRNAs specific for full-length and/or truncated AR-Vs were treated with 1 nM DHT under castrate conditions. Western blots were performed with antibodies specific for the AR NTD or a loading control (ERK-2). (B) Heat-map of the androgen/AR gene expression program (left two columns) with comparison of the responses of these genes to AR-V activity (right two columns). Androgen/AR targets are defined as those genes demonstrating differential expression in variant knock-down cells (siAR exon CE3) treated with DHT vs. vehicle control. (C) Heat-map of the AR-V gene expression program (left two columns) with comparison of the responses of these genes to androgen/AR activity (right two columns). AR-V targets are defined as those genes demonstrating differential expression in cells transfected with siRNA targeting AR exon 7 vs. AR exon 1. (D) Gene set enrichment analysis (GSEA) of “AR-V-specific” or “full-length AR-specific” gene signatures in gene expression datasets supported by AR-Vs (top) or androgen/AR (bottom).

FIG. 19. M-phase cell cycle genes display a biphasic response to both androgen/AR signaling and AR-V signaling. (A) Gene set enrichment analysis (GSEA) of an AR-V-specific gene signatures in gene expression datasets derived from LNCaP cells treated with 1 nM DHT (GSE26483, left) vs. 100 nM DHT (GSE7868, right). (B) LNCaP cells were treated with increasing concentrations of androgens and subjected to quantitative RT-PCR for indicated genes. Bars represent mean+/−S.D. from a triplicate experiment representative of two biological replicates. Western blots were performed using antibodies specific for the AR NTD or loading control (ERK-2). (C) LNCaP cells were infected with increasing titers of lentivirus encoding AR 1/2/3/CE3. RNA and protein analysis was performed as in (B). LNCaP cells were infected with increasing titers of lentivirus encoding AR Δ5/6/7. RNA and protein analysis was performed as in (B).

FIG. 20. Anti androgens achieve on-target inhibition of full-length AR in 22Rv1 cells. (A) 22Rv1 cells were treated under serum-free conditions with 1 nM DHT, 10 μM bicalutamide, or 1 μM enzalutamide as indicated. Quantitative RT-PCR was performed to assess mRNA expression of PSA and hK2. Data represent mean+/−S.D. from 3 biological replicate experiments, each performed in duplicate. (B) mRNA expression of TMPRSS2 in response to 1 nM DHT treatment was assessed as in (A).

FIG. 21. CWR-R1 cells harboring an approximately 48 kb intragenic deletion in AR intron 1 display androgen-independent growth. Cell growth in response to 1 nM DHT was assessed under castrate conditions (CSS) via crystal violet staining at indicated time-points.

FIG. 22. Deletion-enriched CWR-R1 cells are resistant to full-length AR targeting. (A) Multiplex ligation-dependent probe assay with AR-locus specific probes. Binding sites for discrete probes is indicated along the AR gene schematic. Grey boxes represent mean+/−S.D. of all AR locus probes. Probes displaying local loss of signal, indicating gnomic deletion, are indicated. (B) Growth of deletion-enriched CWR-R1 cells following treatment with 10 μM bicalutamide or 1 μM enzalutamide under castrate conditions. (C) Deletion-enriched CWR-R1 cells were transfected with a MMTV-luciferase promoter reporter construct along with indicated siRNAs and treated with combinations of 1 nM DHT, 10 μM bicalutamide, or 1 μM enzalutamide as indicated. Data represent the mean+/−S.D. from at least three independent experiments, each performed in duplicate. Inset: lysates from transfected cells were subjected to Western blot analysis using antibodies targeted to the AR NTD or an internal control (ERK-2). Locations of full-length AR and truncated AR-Vs are indicated.

FIG. 23. Gene set enrichment analysis (GSEA) of a full-length AR-specific gene signature in gene expression datasets derived from LNCaP cells treated with 1 nM (left) vs. 100 nM (right) DHT.

FIG. 24. AR gene rearrangements linked to AR-V expression in CRPC. (A) An 8.7 kb inversion of AR exons 5-7 in passage 2 of the LuCaP 136 xenograft, which was established from CRPC cells in abdominal ascites fluid. mh, microhomology. (B) RT-PCR analysis of AR mRNA in LNCaP cells, LuCaP 86.2 tissue, or LuCaP 136 tissue. Exon organization and relationship with functional protein domains for full length AR (AR-FL) and the ARv567es splice variant is shown. Heteroduplex formation in LuCaP 86.2 PCR products was confirmed. NTD, NH₂-terminal domain; DBD, DNA binding domain; LBD, ligand binding domain. (C) Western blot for the AR NTD or ERK-2 (loading control) in LNCaP cells, LuCaP 86.2 tissue, and LuCaP 136 tissue.

### DETAILED DESCRIPTION OF ILLUSTRATIVE EMBODIMENTS

Prostate cancer (PCa) is a cancer commonly diagnosed in the United States men. For many of these men, the disease can be cured through primary treatments such as surgery or radiation therapy. However, a substantial number of men are diagnosed with locally advanced or metastatic disease, or experience disease recurrence after primary treatment. For over 50 years, the mainstay of treatment for these men has been androgen depletion therapy, which is achieved by suppressing testicular production of androgens, or countering androgen action with antiandrogens.

The target of androgen depletion therapy is the androgen receptor (AR), a 110 kDa member of the steroid receptor transcription factor family. The androgen/AR transcriptional program is required for prostate gland development and normal function of the adult prostate, but it also is required for growth and survival of prostate cancer cells. Therefore, the AR serves as a prototype cell lineage-survival factor in cells of prostatic origin (Garraway and Sellers, 2006 Nat Rev Cancer 6:593-602) and inhibiting the androgen/AR signaling axis often provides effective control of advanced prostate cancer. Prostate cancer is, however, the second leading cause of male cancer death (Siegel et al., 2012 CA Cancer J Clin 62:10-29) due at least in part to progression of the disease to a castration-resistant prostate cancer (CRPC) phenotype.

Resistance to androgen depletion therapy develops through aberrant re-activation of the androgen/AR signaling axis (Attard et al., 2011 Clin Cancer Res 17:1649-57; Chen et al., 2009 Lancet Oncol 10:981-91; Knudsen et al., 2009 Clin Cancer Res 15:4792-8), the effect of which is consistent with the role of AR as a survival factor for cells of prostatic origin. This has been confirmed clinically through the recent re-targeting of persistent AR activity in castration-resistant prostate cancer patients with next-generation agents that block androgen synthesis in testes, adrenal glands, and tumor tissue (e.g., abiraterone; de Bono et al., 2011 N Engl J Med 364:1995-2005; Reid et al., 2010 J Clin Oncol 28:1489-95) or agents that antagonize AR activity even under conditions of AR overexpression (e.g., MDV3100; Scher et al., 2010 Lancet 375:1437-46; Scher et al., 2012 J Clin Oncol 30(suppl 5):abstr LBA1; Tran et al., 2009 Science 324:787-90). In Phase III trials, abiraterone and MDV3100 increased overall survival of castration-resistant prostate cancer patients by 3.9 and 4.8 months, respectively (de Bono et al., 2011 N Engl J Med 364:1995-2005; Scher et al., 2012 J Clin Oncol 30(suppl 5):abstr LBA1). While encouraging, these trials have highlighted two major challenges: 1) pre-existing mechanisms of resistance preclude responses to abiraterone and MDV3100 for nearly half of castration-resistant prostate cancer patients; and 2) resistance can develop rapidly in patients who initially respond to treatment (Scher et al., 2010 Lancet 375:1437-46; Danila et al., 2010 J Clin Oncol 28:1496-501). One barrier to addressing these challenges is that insufficient markers that may be used to guide treatment decisions have been established.

This disclosure describes markers that can identify patients at risk of developing castration-resistant prostate cancer. The markers, and analyses that use the markers, can be used by health professionals to guide treatment decisions by, for example, helping to evaluate the likelihood that a patient will respond to or develop resistance to prostate cancer therapies targeted to the androgen receptor. Thus, in some cases, methods described herein may be used to identify subjects under treatment for prostate cancer as at risk for developing castration-resistant prostate cancer. Such an evaluation may indicate that a change in prescribed therapy is appropriate. In some of these instances, the change may involve modifying the subject's treatment regimen to include administration of a pharmaceutical composition effective for treating castration-resistant prostate cancer before resistance to androgen receptor-based treatments develops.

In the description that follows, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

Truncated AR variant proteins that are involved in castration-resistant prostate cancer typically lack the AR ligand-binding domain, display constitutive, ligand-independent transcriptional activity, and mediate androgen-independent growth of prostate cancer cells in various model systems (Dehm et al., 2008 Cancer Res 68:5469-77; Guo et al., 2009 Cancer Res 69:2305-13; Hu et al., 2009 Cancer Res 69:16-22; Sun et al., 2010 J Clin Invest 120:2715-30; Watson et al., 2010 Proc Natl Acad Sci USA 107:16759-65). Increased expression of the AR3 variant protein (also termed AR-V7; Hu et al., 2009 Cancer Res 69:16-22) in prostate cancer prostatectomy specimens is associated with biochemical recurrence following surgery (Guo et al., 2009 Cancer Res 69:2305-13). In addition, increased mRNA expression of alternatively-spliced AR variants in prostate cancer bone metastases is associated with shorter survival (Hornberg et al., 2011 PLoS One 6:e19059). Therefore, understanding the mechanisms leading to increased synthesis of these species could provide important prognostic information and/or guide more effective use of therapies that inhibit ligand-dependent AR activity.

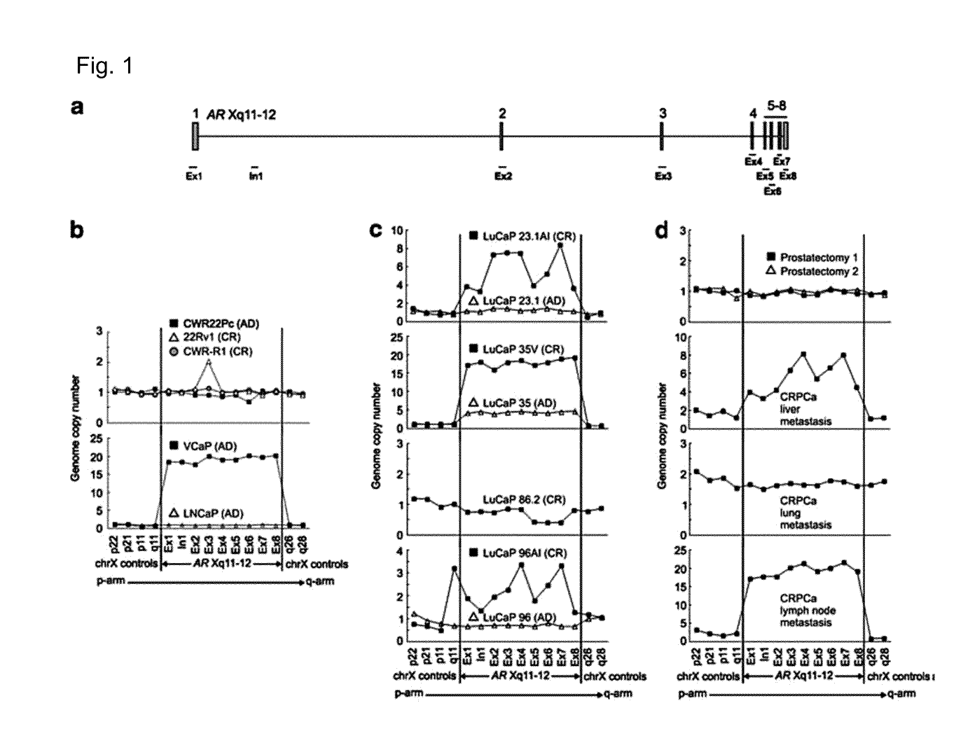

Truncated AR variants proteins were originally discovered and functionally characterized in the castration-resistant prostate cancer cell lines 22Rv1 and CWR-R1 (Dehm et al., 2008 Cancer Res 68:5469-77; Guo et al., 2009 Cancer Res 69:2305-13; Hu et al., 2009 Cancer Res 69:16-22) and the LuCaP 86.2 prostate cancer xenograft (Sun et al., 2010 J Clin Invest 120:2715-30). In 22Rv1 cells, a 35 kb AR intragenic tandem duplication is linked to altered splicing of full-length AR and synthesis of truncated AR variants (Li et al., 2011 Cancer Res 71:2108-17). Previously, we analyzed high-resolution whole-genome copy number data from castration-resistant prostate cancer metastases and discovered a frequent AR copy number imbalance that correlated with AR amplification in castration-resistant prostate cancer (Li et al., 2011 Cancer Res 71:2108-17, U.S. Patent Application Publication No. 2013/0130241 A1). To investigate this phenomenon directly, we employed a multiplex ligation-dependent probe assay (MLPA) with probe sets targeted to coding exons in the AR gene (FIG. 1a). This MLPA approach detected the 22Rv1 duplication involving exon 3, as well as 20-fold amplification of the AR gene in VCaP cells (FIG. 1b). Androgen-dependent prostate cancer tissue obtained from xenografts (FIG. 1c) or clinical specimens (FIG. 1d) displayed one intact genomic copy of the AR gene, with the exception of the LuCaP 35 xenograft, which displayed four genomic copies of the AR gene. However, castration-resistant prostate cancer tissue obtained from xenografts (FIG. 1c) or autopsy specimens (FIG. 1d) displayed frequent AR gene amplification and/or complex patterns of AR gene copy number imbalance. These data suggest that imbalances in AR gene copy number may be important for the progression of castration-resistant prostate cancer.

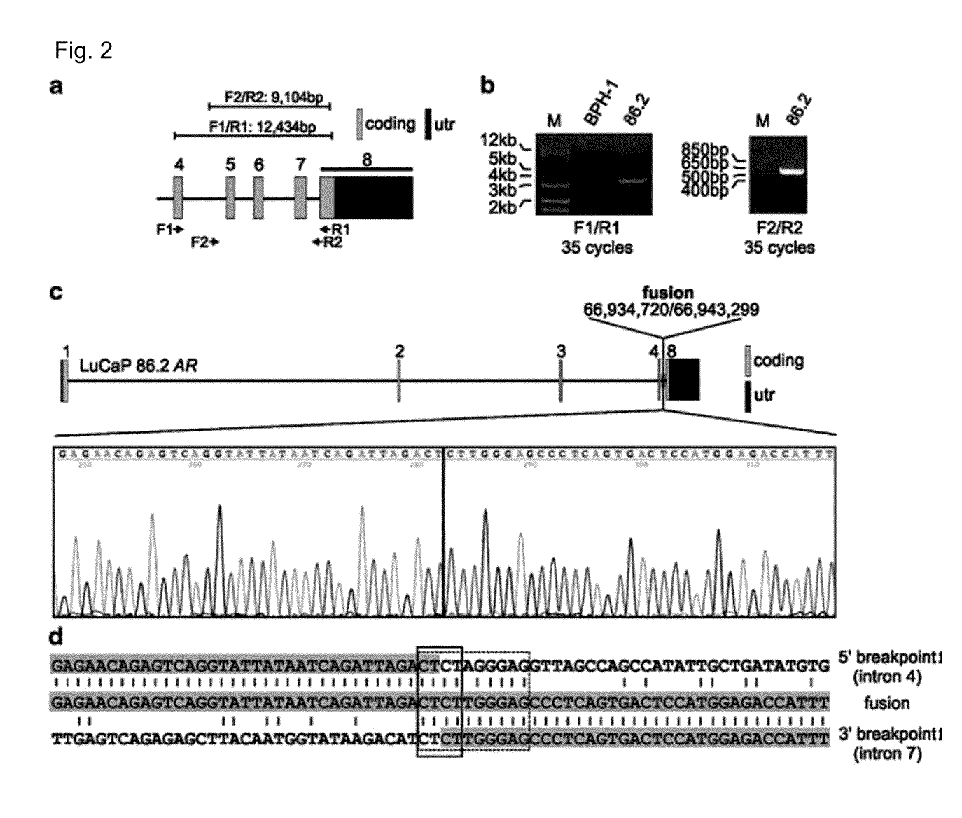

To investigate this phenomenon in more detail, we focused on the LuCaP 86.2 xenograft, which expresses high levels of the truncated AR v567es variant, which arises from the mRNA splicing machinery skipping exons 5, 6, and 7 (Sun et al., 2010 J Clin Invest 120:2715-30). LuCaP 86.2 displayed reduced genome copy number of these exons, indicating a mixed cell population with approximately 50% of cells harboring an intragenic deletion (FIG. 1c). Deletion spanning PCR yielded products consistent with an intragenic deletion encompassing AR exons 5, 6, and 7 (FIGS. 2a and 2b). Sequence analysis verified an 8,579 base pair (bp) deletion (FIG. 2c) with microhomology at the 5′ and 3′ break fusion junctions. This homology suggests a non-homologous end joining mechanism of origin (FIG. 2d). Together, these data identify the AR v567es variant as a marker indicative of castration-resistant prostate cancer and implicate focal intragenic deletion as a novel mechanism underlying synthesis of the truncated AR v567es variant in the LuCaP 86.2 xenograft.

Stable AR mRNA Splicing Alterations in Castration-Resistant Prostate Cancera CWR-R1 Cells

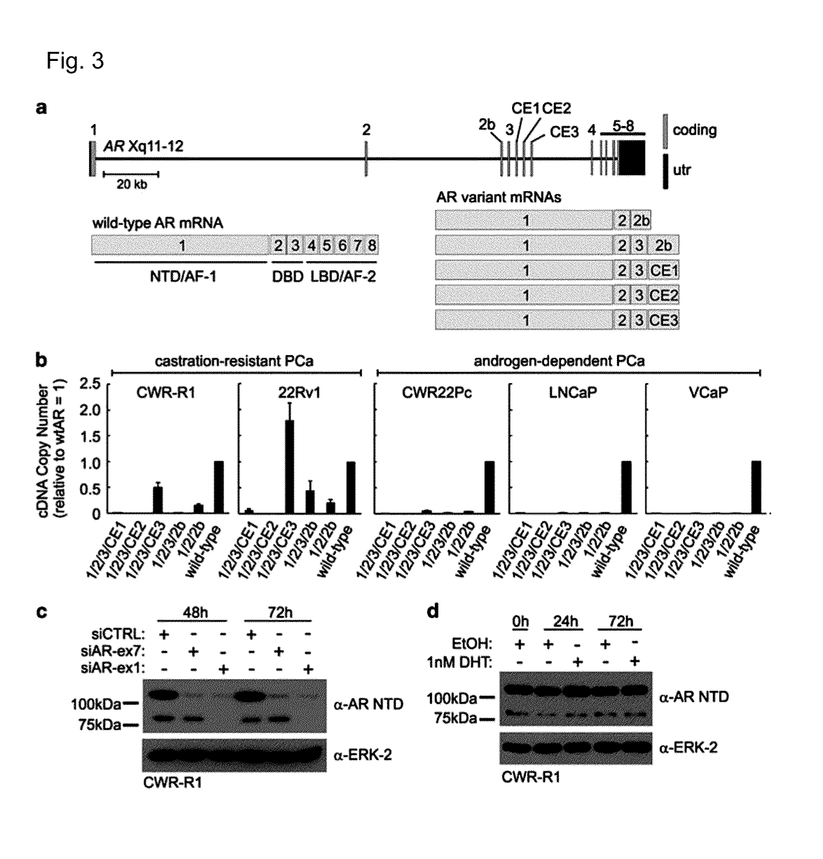

MLPA analysis reveals that the castration-resistant prostate cancer cell line CWR-R1 does not harbor any AR exon copy number alterations, despite previous studies demonstrating that these cells express truncated AR variants (Guo et al., 2009 Cancer Res 69:2305-13). To confirm altered splicing in these cells, we assessed AR mRNA isoform levels using an absolute quantification RT-PCR assay. To correct for variability in AR gene dosage and rates of AR transcription, we scaled copy number for each AR mRNA isoform relative to full-length AR. This approach revealed changes in the ratios of full-length AR mRNA and alternatively-spliced AR isoforms in castration-resistant prostate cancer cells CWR-R1 and 22Rv1 (FIG. 3b). Conversely, androgen-dependent CWR22Pc, LNCaP, and VCaP cells expressed predominantly full-length AR mRNA (FIG. 3b). To test for plasticity in the expression of truncated AR variant expression in CWR-R1 cells, we knocked down full-length AR mRNA using an AR exon 7-targeted siRNA. No changes in truncated AR protein expression were observed following 48 hours or 72 hours of knock-down, whereas an AR exon 1-targeted siRNA completely abolished all AR protein expression in these cells (FIG. 3c). Similarly, no changes in truncated AR variant protein expression were observed in CWR-R1 cells following 24 hours or 72 hours of androgen stimulation (FIG. 3d). These data indicate that the altered AR mRNA splicing pattern in CWR-R1 cells is stable and is unlikely to be an acute cellular response to manipulations of androgen or AR levels.

### Genomic Rearrangements in CWR-R1 Cells and LuCap 136 Cells Identified by Paired-End AR Gene Re-Sequencing

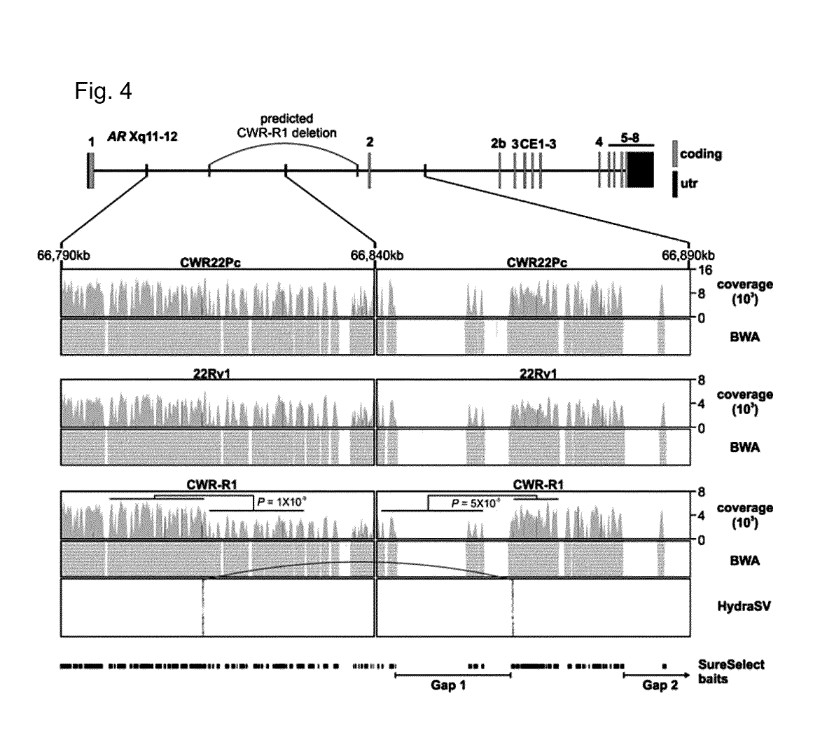

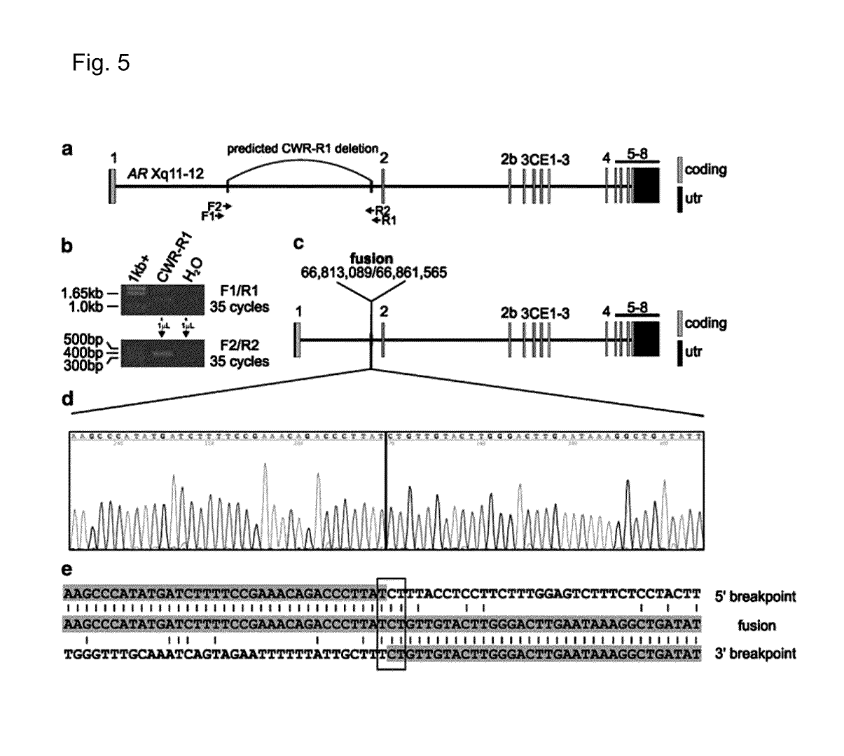

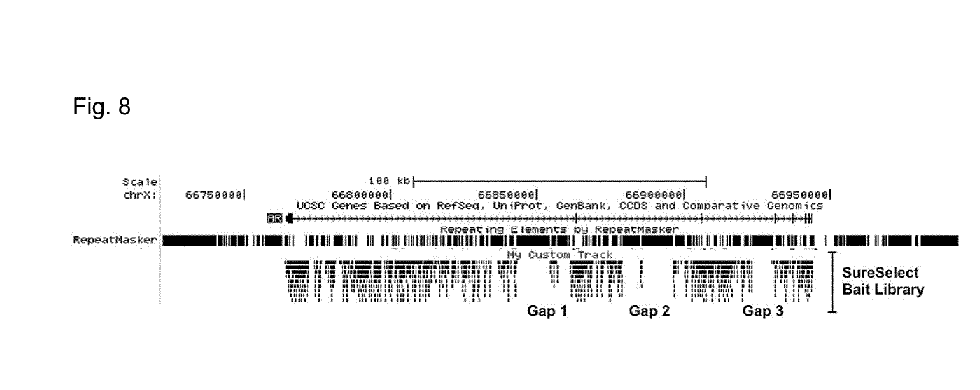

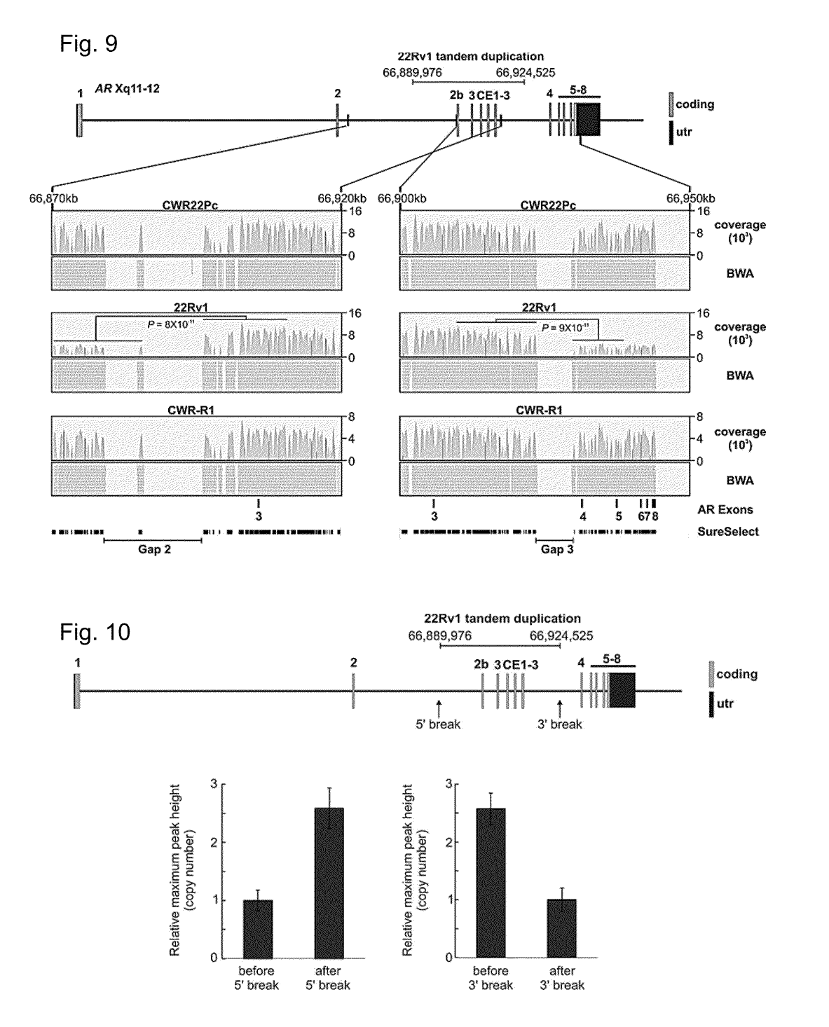

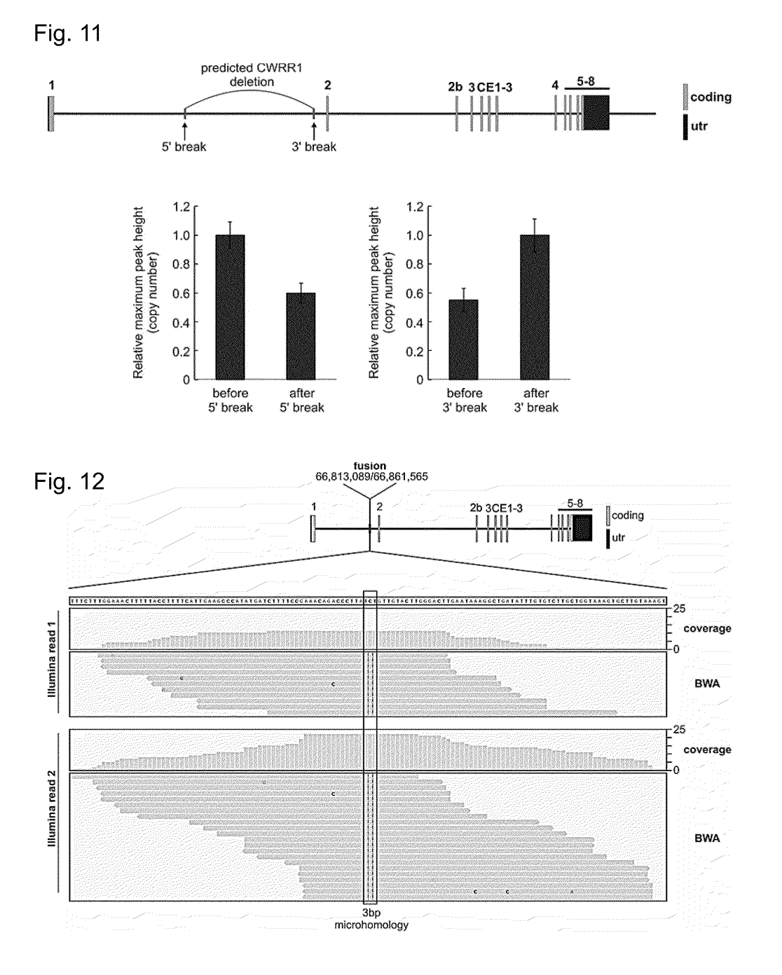

Because MLPA only interrogates AR copy number at coding exons, which represent less than 1.5% of the 180 kb AR gene, we analyzed the nucleotide sequence and structure of the entire AR locus in CWR-R1 cells using a combination of liquid-phase sequence capture and Illumina paired-end massively parallel sequencing (FIG. 8). Androgen-dependent CWR22Pc and castration-resistant prostate cancer 22Rv1 cells were sequenced concurrently. Strikingly, structural variant analysis of paired-end reads using the Hydra workflow (Quinlan et al., 2010 Genome Res 20:623-35) identified an approximately 48 kb intragenic deletion within AR exon 1 in a sub-population of the CWR-R1 cell line, which was also apparent from a relative decrease in sequence coverage peak height within this region (FIG. 4 and FIG. 11). To confirm this structural alteration in CWR-R1 cells, we performed nested PCR using primers spanning the deletion (FIGS. 5a and 5b). Sanger sequencing of cloned PCR products revealed deletion of 48,476 bp from AR intron 1 (FIGS. 5c and 5d). Alignments of the 5′ and 3′ break fusion junctions revealed 3 bp of microhomology, suggesting non-homologous end joining as the mechanism underlying this deletion (FIG. 5e).

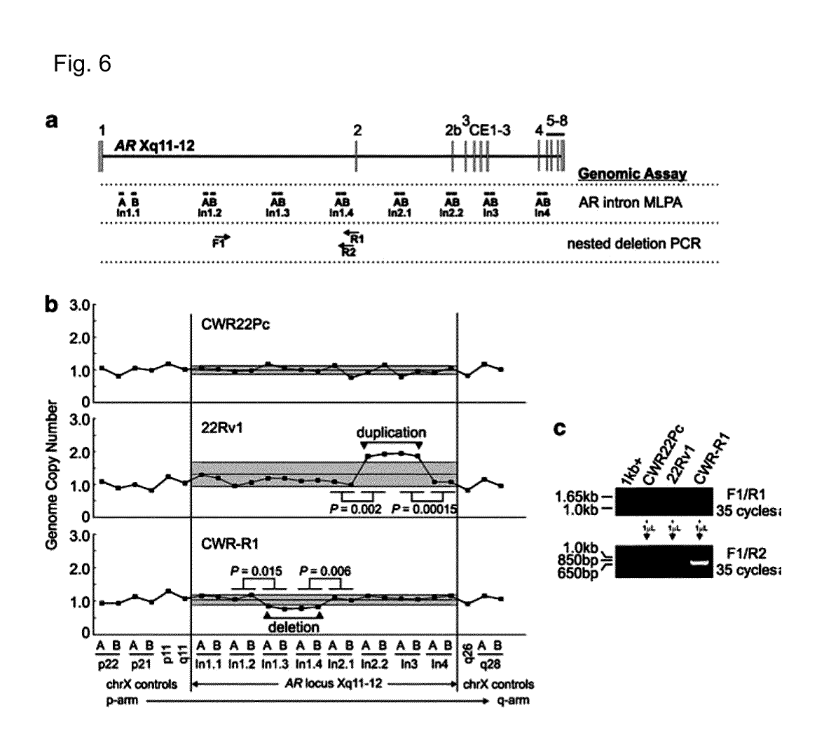

To quantify the prevalence of this deletion, we performed MLPA with probe pairs custom-designed to query copy number at regular intervals along the length of the AR gene (FIG. 6a). MLPA probe pairs targeted within this approximately 48 kb region displayed an approximately 20%-30% decrease in copy number (FIG. 6b). There were no copy number alterations in this region detected by MLPA in CWR22Pc or 22Rv1 cells (FIG. 6b), and nested PCR with deletion-spanning primers did not generate products in CWR22Pc or 22Rv1 cells (FIG. 6c). Similarly, only CWR-R1 cells yielded 76 bp Illumina sequencing reads that could be aligned to a 130 bp template harboring this specific breakpoint sequence (FIG. 12). Together, these data identify this 48,476 bp deletion within intron 1 of the AR gene as a marker for castration-resistant prostate cancer, and that the marker is present in 20%-40% of the cells in the CWR-R1 cell line.

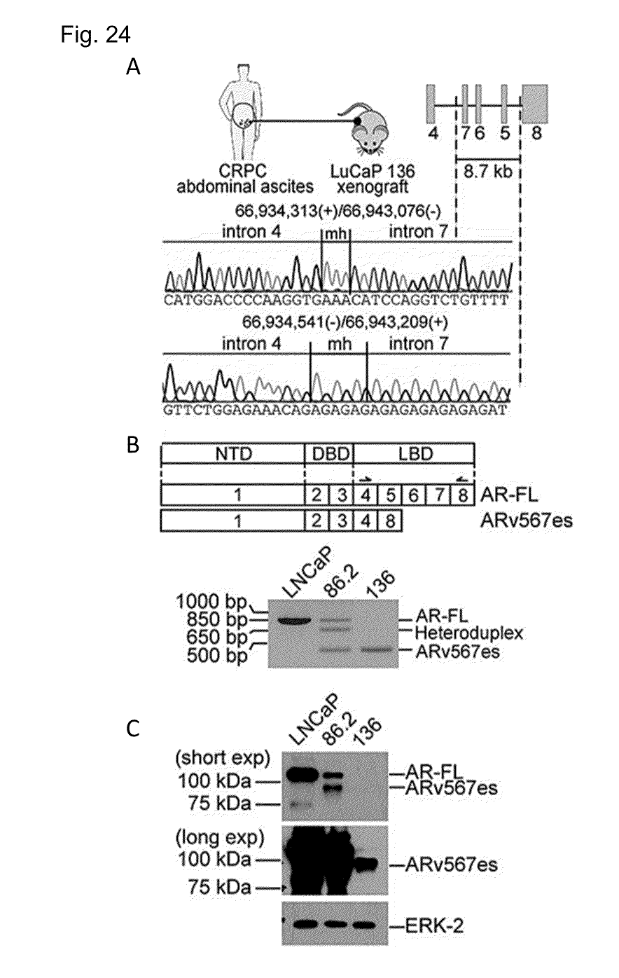

A prostate cancer xenograft established from CRPC abdominal ascites, LuCaP 136, expresses ARv567es mRNA and protein (Kumar et al., 2011. Proc Natl Acad Sci 108(41):17087-17092; Sun et al. 2010. J Clin Invest 120(8):2715-2730.). Whole exome re-sequencing of LuCaP 136 genomic DNA has been performed (Kumar et al., 2011. Proc Natl Acad Sci 108(41):17087-17092), but this did not provide an obvious basis for ARv567es expression, and intragenic deletion of AR exons 5-7 was not detected using PCR. Therefore, we re-sequenced the 183 kb AR gene in LuCaP 136 genomic DNA via hybrid capture followed by Illumina-based massively parallel paired-end sequencing. This analysis revealed a copy-neutral 8.7 kb inversion encompassing AR exons 5-7 (FIG. 24A). In contrast to heterogeneous AR expression in LuCaP 86.2 (FIGS. 24B and C), early-passage LuCaP 136 tissue displayed exclusive expression of ARv567es mRNA (FIG. 24B) and protein (FIG. 24C), which was consistent with very few cells harboring a normal AR allele. Although there was no archival patient material corresponding to LuCaP 136, discovery of this intragenic AR inversion was made in tissue that had been propagated for only two passages in non-castrate male mice. Later passages of LuCaP 136, which were serially-propagated in non-castrate male mice, displayed coordinate loss of cells with this AR exon 5-7 inversion allele and AR v567es protein expression.

### Enrichment for Cells Harboring AR Intron 1 Deletion During Castration

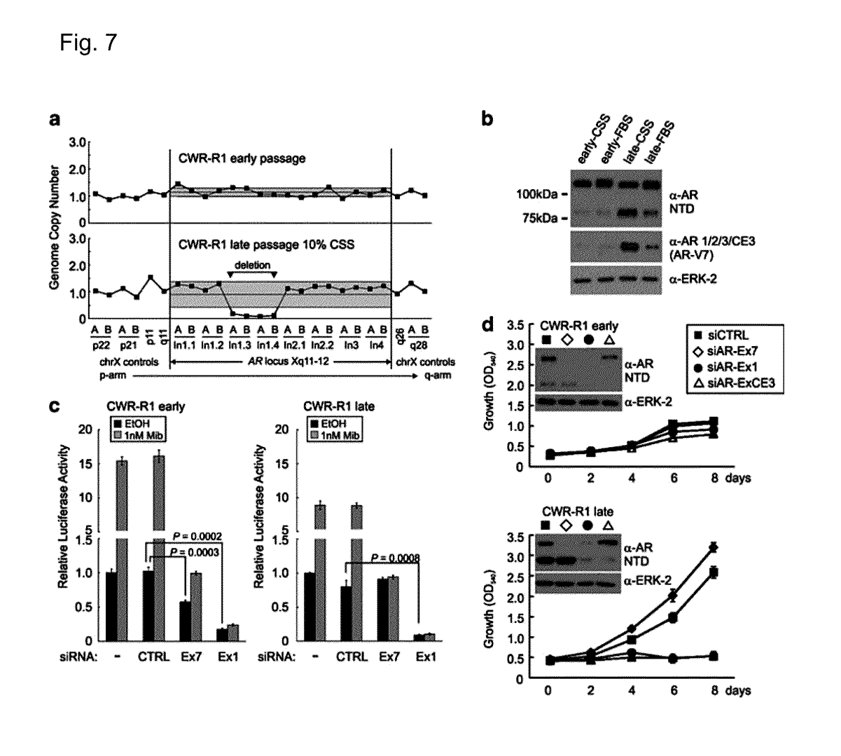

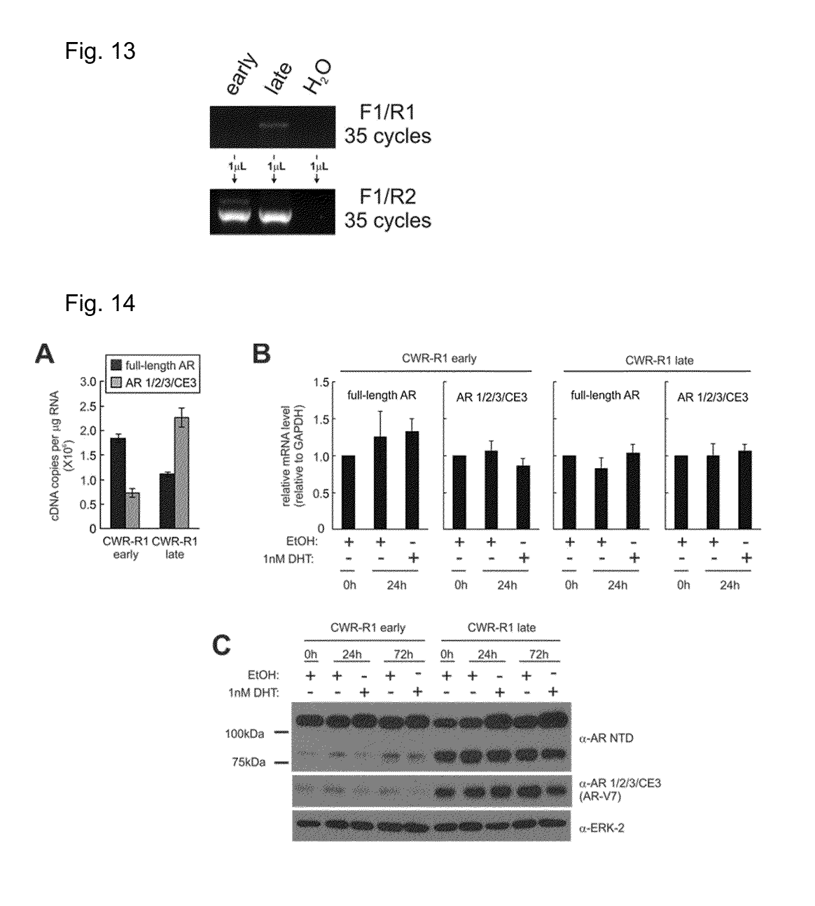

The levels of truncated AR isoforms expressed in CWR-R1 cells are markedly higher when these cells are grown as xenografts in castrated mice versus intact mice (Guo et al., 2009 Cancer Res 69:2305-13). All the analyses in our study had been performed with CWR-R1 cells that had been cultured in complete medium (contains androgens) for 10-20 passages, which would allow the growth of both androgen-dependent and castration-resistant prostate cancer cell populations. Therefore, we compared AR gene structure in early passage CWR-R1 cells (referred to as CWR-R1 early) to CWR-R1 cells that were cultured in the absence of androgens for 20 passages (referred to as CWR-R1-late). Remarkably, no copy number decrease within AR intron 1 was apparent following MLPA analysis of CWR-R1 early cells (FIG. 7a), despite a positive nested PCR signal for this deletion (FIG. 13). Conversely, MLPA probe signal in this region was nearly completely lost in CWR-R1 late cells (FIG. 7a), indicating that this deletion was a marker of the castration-resistant prostate cancer cell sub-population.

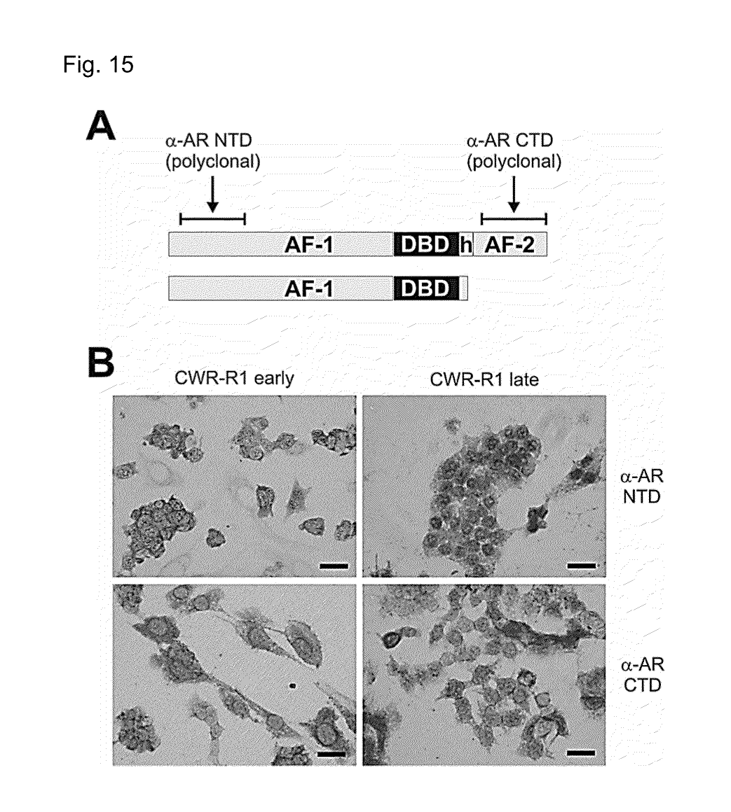

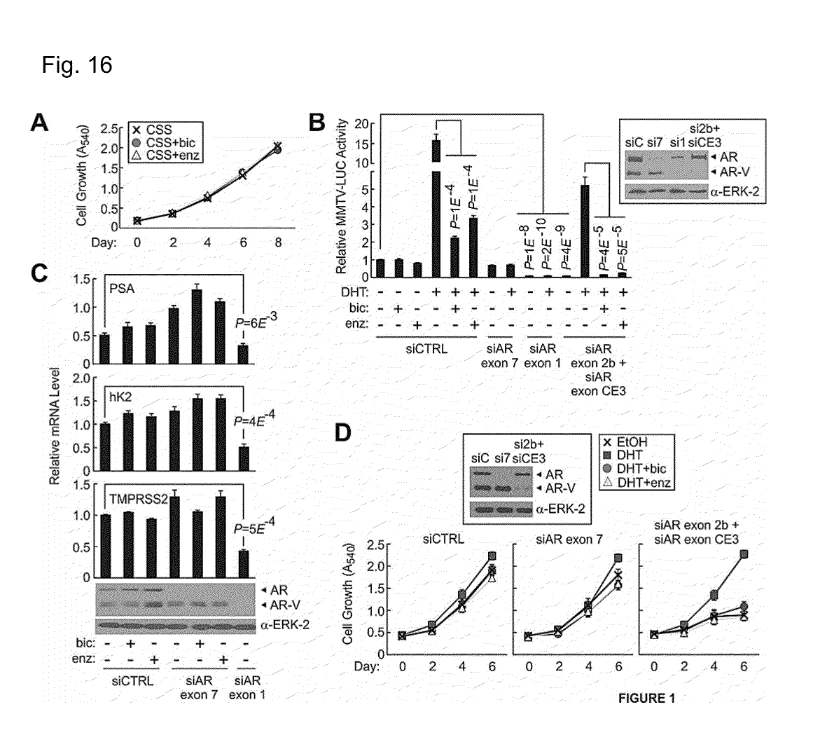

Consistent with this deletion underlying the AR splicing patterns in CWR-R1 cells, expression of the truncated AR 1/2/3/CE3 variant (also referred to as AR-V7 (Hu et al., 2009 Cancer Res 69:16-22) or AR3 (Guo et al., 2009 Cancer Res 69:2305-13)) was low in CWR-R1 early cells, regardless of whether they were cultured in whole serum or steroid-depleted serum (FIG. 7b). However, CWR-R1 late cells displayed high-level expression of AR 1/2/3/CE3 protein (FIG. 7b). These changes at the protein level corresponded with a stable shift in splicing favoring the AR 1/2/3/CE3 AR mRNA isoform in CWR-R1 late versus CWR-R1 early cells (FIG. 14). CWR-R1 cells cultured in the presence of androgens for 20 passages displayed an intermediate AR 1/2/3/CE3 protein expression pattern (FIG. 7b). Immunostaining of cells grown under castrate conditions demonstrated increased nuclear expression of the AR amino-terminal domain versus the AR carboxy-terminal domain in CWR-R1 late cells, but not CWR-R1 early cells (FIG. 15). Together, these data demonstrate that cells harboring this approximately 48 kb deletion within AR exon 1 display a splicing switch that favors stable, high-level expression of the truncated AR 1/2/3/CE3 variant.

To investigate whether there may be functional consequences to these differences in truncated AR variant expression, we examined AR transcriptional activity in CWR-R1 early and CWR-R1 late cells. AR transactivation in response to the synthetic androgen mibolerone was higher in CWR-R1 early cells than CWR-R1 late cells and knock-down of full-length AR inhibited this androgen response in both cell lines (FIG. 7c). Interestingly, knock-down of AR expression with siRNAs targeted to either AR exon 1 or exon 7 inhibited androgen-independent transcriptional activity in CWR-R1 early cells, but only siRNA targeted to AR exon 1 had this effect in CWR-R1 late cells (FIG. 7c). These data indicate that androgen independent AR activity in CWR-R1 early cells is dependent on full-length AR expression, whereas androgen-independent AR activity in CWR-R1 late cells is mediated by truncated AR variants through a mechanism that is independent of full-length AR. To investigate differential siRNA sensitivity and a differential role for full-length AR in more detail, we compared the androgen-independent growth of CWR-R1 early and CWR-R1 late cells transfected with AR-targeted siRNAs. Consistent with their selection under castrate conditions, CWR-R1 late cells displayed a rapid androgen-independent growth rate which was inhibited by siRNA targeted to AR exon 1 but not AR exon 7 (FIG. 7d). Conversely, CWR-R1 early cells grew slowly under androgen-independent conditions during this short time-course and there was limited response to AR-targeted siRNA (FIG. 6d).

We have therefore defined two additional simple AR gene structural alterations that are linked to the pathologic AR splicing patterns in the models of prostate cancer progression. These data, combined with MLPA analysis of additional castration-resistant prostate cancer specimens, indicate that the prevalence of AR gene alterations in tumors resistant to androgen depletion therapy may be higher than previously anticipated. While targeted methods such as MLPA are useful for identifying deletions or duplications that involve probe binding sites, this study has illustrated that unbiased evaluation of the entire AR gene sequence and structure is a preferable approach.

Previous analysis of genome-wide copy number data from clinical castration-resistant prostate cancer specimens suggested that complex patterns of copy number gain and copy number loss occurred along the length of the AR gene (Li et al., 2011 Cancer Res 71:2108-17), which is supported by MLPA analysis in this study. Here, we demonstrated that large deletions involving intron 1 are associated with enhanced synthesis of the truncated AR 1/2/3/CE3 variant (also referred to as AR-V7 or AR3) and a growth advantage under castrate conditions. In the CWR-R1 model, castration-mediated enrichment for cells harboring intron 1 deletion resulted in an overall population that exhibited levels of AR 1/2/3/CE3 that were equivalent to or greater than the levels of full-length AR. This is important because a recent study of surgical specimens of castration-resistant prostate cancer bone metastases with an antibody specific for the AR NTD demonstrated that protein expression of truncated AR variants can reach similar high levels relative to full-length AR (Hornberg et al., 2011 PLoS One 6:e19059). Moreover, patients with castration-resistant prostate cancer bone metastases that displayed the highest levels of alternatively-spliced, truncated AR mRNA variants had shorter cancer-specific survival after metastasis surgery than other castration-resistant prostate cancer patients (Hornberg et al., 2011 PLoS One 6:e19059). Therefore, increased expression of truncated AR variants is an important component of clinical prostate cancer progression. The data in this study strongly suggests that alterations in the architecture of the AR gene may underlie these disruptions in normal splicing patterns.

Overexpression of the AR 1/2/3/CE3 variant (also referred to as AR-V7 or AR3), the AR v567es variant, or a truncated AR variant of mouse origin (mAR-V4) in LNCaP cells can induce androgen-independent expression of AR target genes and growth under castrate conditions in vitro and in vivo (Guo et al., 2009 Cancer Res 69:2305-13; Sun et al., 2010 J Clin Invest 120:2715-30; Watson et al., 2010 Proc Natl Acad Sci USA 107:16759-65). In this study, knock-down of full-length AR had no effect on androgen-independent AR activity or androgen-independent growth in late-passage CWR-R1 cells. Knock-down of a truncated AR variant AR 1/2/3/CE3, however, inhibited these parameters. We have also demonstrated this differential response to isoform-targeted siRNAs in the 22Rv1 cell line (Dehm et al., 2008 Cancer Res 68:5469-77). Conversely, early-passage CWR-R1 cells displayed modest androgen-independent growth and measurable androgen-independent AR activity, which was inhibited following knock-down of full-length AR. These data demonstrate that the CWR-R1 cell line is heterogeneous and that growth conditions can have dramatic effects on the relative proportions of androgen-dependent cells and castration-resistant prostate cancer cells, which may explain a previous report in which CWR-R1 cells displayed decreased proliferation and increased apoptosis in response to full-length AR knock-down (Guo et al., 2009 Cancer Res 69:2305-13). With this in mind, the LuCaP 86.2 xenograft tissue evaluated in this study was propagated in an intact male mouse and MLPA data reflected an approximate 50/50 mixture of cells with either one intact AR gene copy or one AR gene copy reflecting a 8,579 bp deletion in exons 5, 6, and 7. If the cell population harboring the 8,549 bp intragenic deletion is indeed the cell population which synthesizes the AR v567es variant, these cells would not be able to synthesize full-length AR and would be truly independent of full-length AR activity (Sun et al., 2010 J Clin Invest 120:2715-30).

### AR-Vs are Sufficient for Resistance to Enzalutamide in 22Rv1 Cells

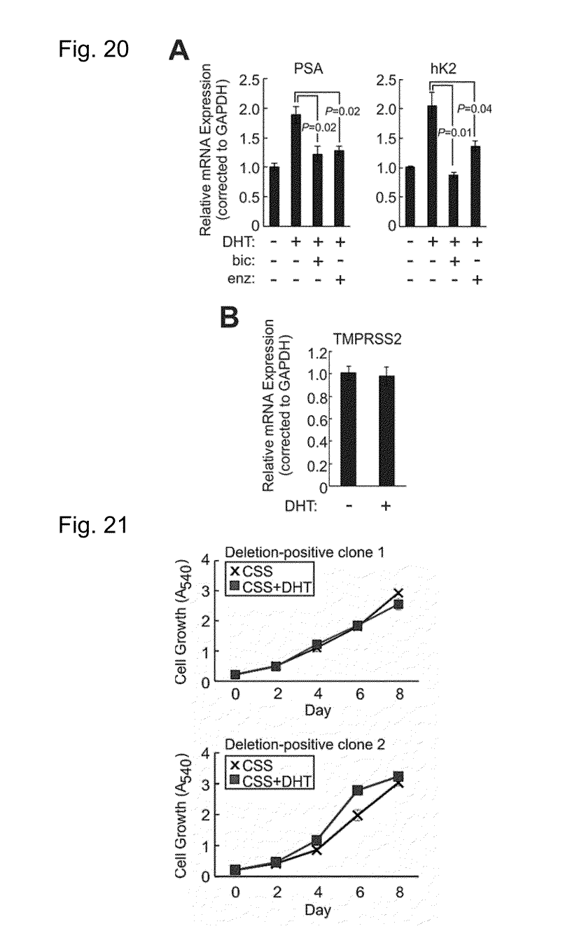

The castration-resistant prostate cancer cell line 22Rv1 is characterized by a 35 kb tandem duplication encompassing AR exon 3 (Li et al., 2011 Cancer Res 71:2108-17). This rearrangement is associated with enhanced mRNA and protein expression of truncated AR variants AR 1/2/3/2b and AR 1/2/3/CE3 (also referred to as AR-V7/AR3) (Chan et al., 2012 J Biol Chem 287:19736-49; Guo et al., 2009 Cancer Res 69:2305-13; Hu et al., 2009 Cancer Res 69:16-22; Li et al., 2011 Cancer Res 71:2108-17). 22Rv1 cells display robust growth under castrate conditions, which was unaffected by anti-androgens bicalutamide or enzalutamide (FIG. 16A). Both bicalutamide and enzalutamide are able to antagonize androgen-mediated activation of the AR target genes PSA and hK2 (FIG. 20A) as well as an AR-responsive mouse mammary tumor virus (MMTV)-luciferase reporter (FIG. 16B), showing that these drugs can achieve on-target inhibition of full-length AR in these cells. Similarly, androgen-induced MMTV activity was blocked following selective knock-down of full-length AR (FIG. 16B). However, knock-down of AR variants resulted in robust inhibition of constitutive, androgen-independent MMTV-LUC activity. Similarly, constitutive, androgen-independent expression of PSA and hK2 was blocked by AR variant knock-down, but not by manipulations that block full-length AR (bicalutamide, enzalutamide, full-length AR knock-down, or AR knock-down combined with antiandrogens, FIG. 16C). Interestingly, constitutive AR variant activity appeared to maintain TMPRSS2 expression at a maximal AR-inducible level, as there was no response to androgens (FIG. 20B), but expression was inhibited by AR variant knock-down (FIG. 16C). Together these data show that AR variants are independent effectors of constitutive AR transcriptional activity in these cells.

Next, we assessed the effects of androgens and anti-androgens on the growth of 22Rv1 cells under conditions of full-length versus AR variant knock-down. Remarkably, knock-down of AR variants, but not full-length AR, reduced the androgen-independent growth rate of these cells and restored robust growth-responsiveness to androgens (FIG. 16D). Perhaps more importantly, AR variant knock-down restored the ability of anti-androgens to inhibit this newly-acquired androgen-dependent growth phenotype (FIG. 16D). Based on this finding, we conclude that AR variants are sufficient for resistance of 22Rv1 cells to therapies targeting full-length AR, including enzalutamide.

### AR Gene Rearrangements Mark AR Variant-Driven, Enzalutamide-Resistant Cells in Heterogenous PCa Cell Populations

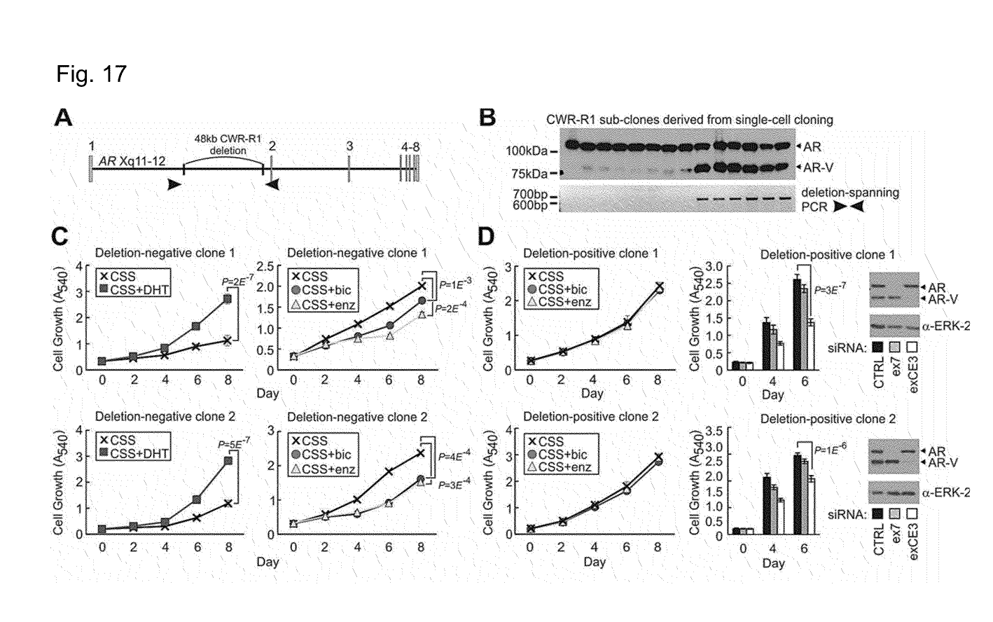

Single cell cloning revealed that cells positive for the approximately 48 kb deletion to AR intron 1 displayed high-level expression of the AR 1/2/3/CE3 variant (FIG. 17B). Conversely, cells that were negative for the approximately 48 kb deletion expressed predominantly full-length AR (FIG. 17B). Subclones negative for the approximately 48 kb AR intragenic deletion displayed a basal level of androgen-independent growth which was enhanced by DHT (FIG. 17C). This basal level of androgen-independent growth was reduced by treatment with bicalutamide or enzalutamide, indicating that full-length AR was required for the androgen-independent growth of these cells (FIG. 17C). Conversely, subclones positive for the approximately 48 kb AR intragenic deletion displayed rapid androgen-independent growth which was unaffected by androgens (FIG. 21) or anti-androgens (FIG. 17D). Selective knock-down of AR 1/2/3/CE3, however, inhibited androgen-independent growth of these rearrangement-positive cells (FIG. 17D).

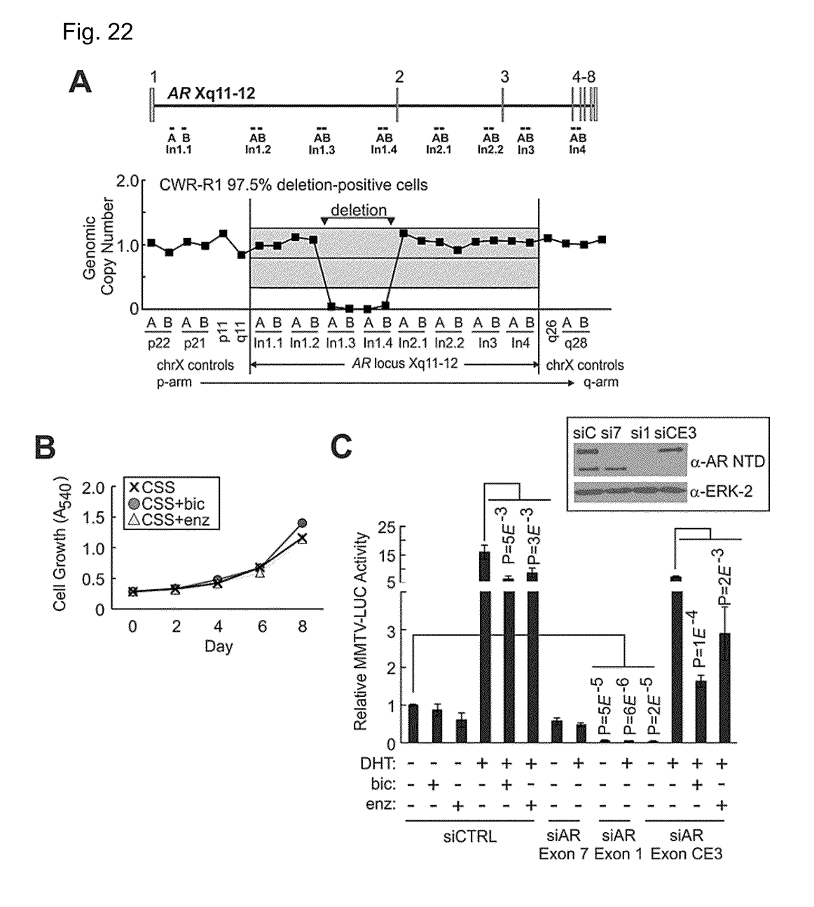

To verify that this property of enzalutamide resistance was not restricted to a few rare cells, we tested the effects of anti-androgens on a version of the CWR-R1 cell line that had been propagated long-term under castrate conditions. Long-term castration enriches for the AR intron 1 deletion-positive population (FIG. 7a, FIG. 22A). Similar to 22Rv1 cells, androgen-independent growth of deletion-enriched CWR-R1 cells was insensitive to bicalutamide and enzalutamide (FIG. 22B). Nevertheless, anti-androgens were able to achieve on-target activity and inhibit androgen-induced MMTV-LUC activation (FIG. 22C). However, only AR variant knock-down was able to inhibit constitutive, androgen-independent MMTV activity (FIG. 22C). Therefore, we conclude that the approximately 48 kb AR intron 1 deletion can discriminate between individual cells in the heterogeneous CWR-R1 cell line that are enzalutamide-responsive and cells that are driven by AR variant activity and resistant to inhibition of full-length AR.

### AR Variants are Independent Effectors of the Androgen/AR Transcriptional Program.

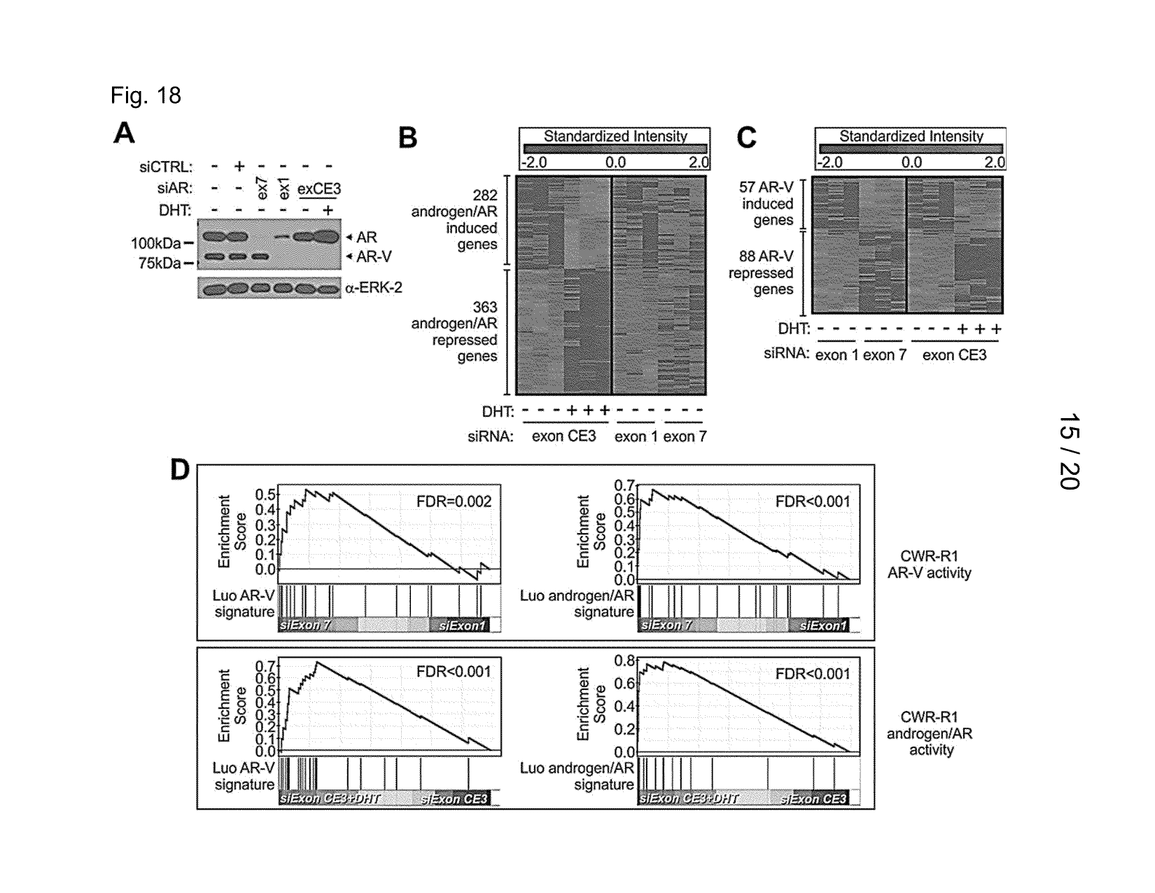

AR variants have been reported to induce unique transcriptional targets such as AKT1 (Guo et al., 2009 Cancer Res 69:2305-13), which may play a role in enzalutamide resistance (17CR). In AR intron 1 deletion-enriched CWR-R1 cells, however, we did not observe any changes in AKT1 expression following AR 1/2/3/CE3 knock-down. Therefore, to understand the mechanistic basis for AR variant-mediated resistance to enzalutamide, we performed gene expression profiling of deletion-enriched CWR-R1 cells. Because constitutive activity of AR variants can mask androgen/AR induction targets (and vice versa) we assessed the androgen/AR transcriptional program following AR 1/2/3/CE3 knock-down and assessed the AR variant transcriptional program following full-length AR knock-down (FIG. 18A). Many, but not all, of the genes responsive to androgen/AR activity were similarly activated/repressed in a constitutive manner by AR 1/2/3/CE3 in these cells (FIG. 18B). This suggests that the AR variant transcriptional program represented a subset of the broader androgen/AR transcriptional program. Indeed, when we focused on AR variant responsive genes, nearly all were regulated in the exact same manner by androgen/AR activity (FIG. 18C). These data confirm that AR variants are constitutive and independent effectors of the AR transcriptional program, which explains why androgens and AR variants can support maximal growth of the same cell line in an interchangeable fashion (FIG. 16D).

These findings are in contrast to reports suggesting that AR variants have gene signatures distinct from full-length AR, including a set of genes involved in M-phase cell cycle progression (Hu et al., 2012 Cancer Res 72:3457-62). To understand the basis for this discrepancy, we used gene set enrichment analysis (GSEA) (Subramanian et al., 2005 Proc Natl Acad Sci USA 102:15545-50) to test the response of this “AR-V-specific” set of M-phase-genes (Hu et al., 2012 Cancer Res 72:3457-62) in CWR-R1 cells. This AR variant-responsive M-phase gene set was positively-enriched in both androgen/AR and AR variant gene expression datasets derived from CWR-R1 cells (FIG. 18D). Similarly, a gene set deemed to be “full-length AR-specific” (Hu et al., 2012 Cancer Res 72:3457-62) was positively-enriched in both of these CWR-R1-derived gene expression datasets (FIG. 18D). Therefore, these signatures could not discriminate between AR variant versus androgen/AR activity in CWR-R1 cells.

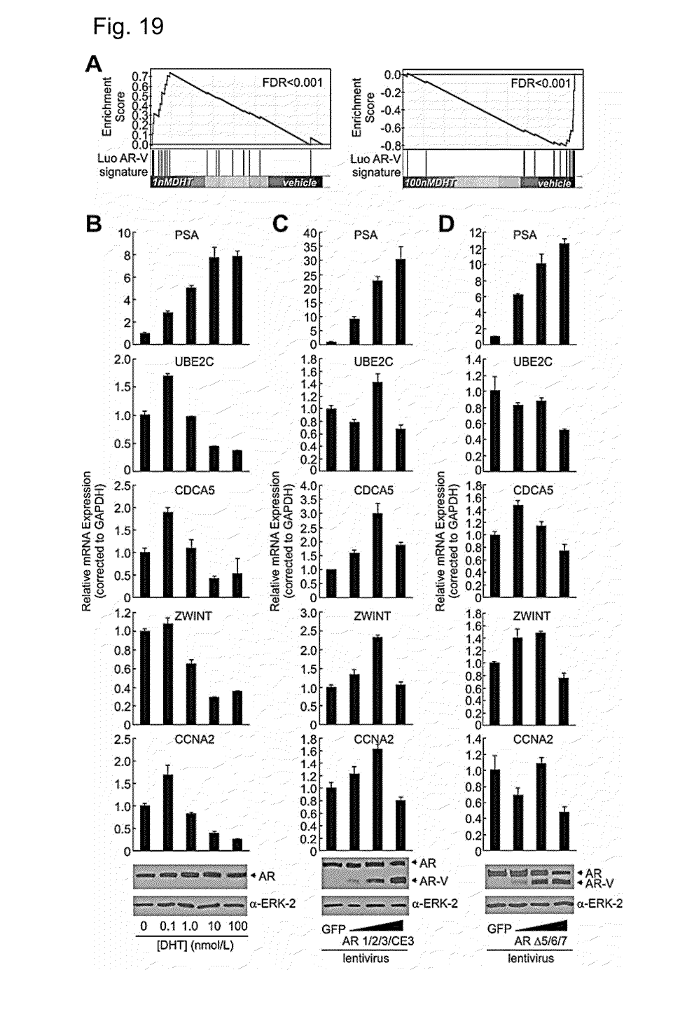

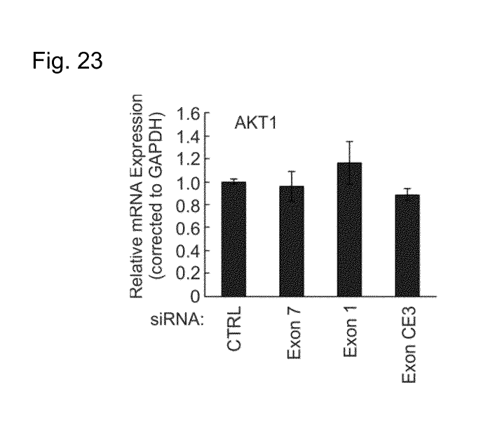

AR variants have been shown to drive biphasic AR signaling in a manner similar to androgens (Chan et al., 2012 J Biol Chem 287:19736-49). Therefore, differences previously noted between AR variants and full-length AR transcriptional programs could have arisen from comparing different strengths of AR transcriptional output from AR variants vs. androgens. To test this, we performed GSEA with gene expression datasets derived from LNCaP cells treated with 1 nM DHT (a pro-proliferative dose) or 100 nM DHT (an anti-proliferative dose). The “full-length AR” signature displayed positive enrichment in both the 1 nM DHT and 100 nM DHT gene expression datasets (FIG. 23). Conversely, the “AR-V-specific”signature displayed positive enrichment in the 1 nM DHT dataset, but strong negative enrichment in the 100 nM DHT dataset (FIG. 19A). Therefore, these data indicate that the “AR-V-specific” signature does not discriminate between AR variant versus full-length AR signaling, but rather reflects proliferative versus growth suppressive levels of AR signaling output. To test this further, we treated LNCaP cells with androgens at concentrations that cover the range of proliferative and growth suppressive doses (0.1 nM DHT to 100 nM DHT) and assessed expression of M-phase genes UBE2C, CDCA5, ZWINT, and CCNA2. Whereas PSA expression increased concomitant with increasing androgen concentration, all of the M-phase specific genes displayed a biphasic response: induction at low androgen concentrations and/or repression at higher doses (FIG. 19B). Similarly, when increasing titers of lentivirus encoding the AR 1/2/3/CE3 (AR-V7/AR3) variant (FIG. 19C) or the AR Δ5/6/7 (ARv567^{es}) variant (FIG. 19D) were used for infection, similar biphasic responses were observed for M-phase-specific genes, but not PSA. Therefore, these data challenge the notion that AR variants have acquired unique transcriptional targets and provide strong support for the concept that AR variants are independent effectors of the androgen/AR transcriptional program.

In summary, this disclosure describes intragenic deletions involving coding and non-coding sequences in the AR gene in castration-resistant prostate cancer, which we have linked to expression of truncated AR variants that support the castration-resistant prostate cancer phenotype. Thus, structural alterations in the AR gene may represent a widespread yet previously unanticipated mechanism of therapy resistance in prostate cancer. Consequently, truncated AR variants may serve as markers for individuals at risk of developing castration-resistant prostate cancer.

Moreover, AR variant expression driven by AR gene rearrangements can mediate resistance to therapies targeting full-length AR, including next-generation anti-androgens such as, for example, enzalutamide/MDV3100. Resistance to such therapies, either de novo or acquired during therapy, is a significant clinical limitation for new AR axis inhibitors (Scher et al., 2010 Lancet 375:1437-46; Danila et al., 2010 J Clin Oncol 28:1496-501). Importantly, many patients who display disease progression on enzalutamide also display rising PSA, indicating that enzalutamide-resistant tumors remain driven by persistent AR activity (Scher et al., 2012 N Engl J Med 367:1187-97). AR variants are overexpressed in a subset of castration-resistant prostate cancer metastases and correlate with poor survival (Hornberg et al., 2011 PLoS One 6:e19059). Mechanistically, our data demonstrate that AR variants mediate enzalutamide resistance in castration-resistant prostate cancer through their activities as independent effectors of the AR transcriptional program, driving persistent activation of a large subset of AR target genes at a level of output sufficient to support cell proliferation. Overall, these results establish a foundation for reversing enzalutamide resistance by inhibiting expression of AR variants and/or AR activity.

Thus, in one aspect, this disclosure describes a method for detecting expression of an androgen receptor (AR) variant. Generally, the method includes analyzing a biological sample obtained from a subject, wherein the biological sample includes cells that express a plurality of non-wild-type androgen receptor polynucleotides. Each non-wild-type androgen receptor polynucleotide can be characterized in terms of a copy number. The copy number is typically a genomic copy number—i.e., the number of copies of genomic DNA that can be transcribed to produce a non-wild-type androgen receptor variant polynucleotide. Typically, the cells can be of prostatic origin. One can measure the genomic copy number of at least one polynucleotide that can be transcribed to produce a non-wild-type androgen receptor polynucleotide and identify the sample as exhibiting expression of an AR variant if the measured polynucleotide exhibits a copy number that differs from the mean AR copy number by at least one standard deviation.

In some embodiments, the non-wild-type androgen receptor polynucleotide can include at least a portion of AR intron 1. In some of these embodiments, the AR variant can include a sufficient genomic duplication of at least part of AR intron 1 so that the copy number can be greater than the mean AR copy number by at least one standard deviation. In other embodiments, the AR variant can reflect a deletion of a sufficient portion of AR intron 1 so that copy number can be less than the mean AR copy number by at least one standard deviation. In some of these embodiments, the AR variant can reflect a 48,476 bp deletion from AR intron 1 as shown in FIG. 6b-6e.

In other embodiments, the non-wild-type androgen receptor polynucleotide can reflect a deletion of at least a portion of AR exon 5, AR exon 6, or AR exon 7. In some of these embodiments, the non-wild-type androgen receptor polynucleotide can reflect an 8579 bp deletion of AR exon 5, AR exon 6, and a portion of AR exon 7, as shown in FIG. 2c and FIG. 2d.

In still other embodiments, the non-wild-type androgen receptor polynucleotide can include AR variants known as AR 1/2/2b, AR 1/2/3/2b, AR 1/2/3/CE1, AR 1/2/3/CE2, AR 1/2/3/CE3, or ARv567es.

In another aspect, this disclosure describes a method for detecting expression of an AR variant in a subject. Generally, the method includes obtaining a biological sample from a subject that includes cells of prostatic origin and then sequencing a sufficient portion of the AR genomic DNA from at least one cell to detect a linear rearrangement of the AR genomic DNA. The linear rearrangement of the AR genomic DNA is indicative of an AR variant that can give rise to castration-resistant prostate cancer. Models that exhibit levels of truncated AR variant expression sufficient to drive the castration-resistant prostate cancer phenotype (e.g., 22Rv1, CWR-R1, and LuCaP 86.2) can exhibit different variations in the AR gene template. The exemplary AR intragenic duplications and deletions that we have defined appear to result from homologous recombination-independent mechanisms such as, for example, microhomology-mediated break-induced replication in 22Rv1, non-homologous end joining in LuCaP 86.2, CWR-R1, and LuCap 136. Therefore, the exact locations of breakpoints in the AR locus may not necessarily be conserved between specimens. Each of these three models of castration-resistant prostate cancer, however, displays a unique splicing signature and repertoire of truncated AR variant protein expression. Therefore, different patterns of AR gene alteration may give rise to different AR splicing patterns in clinical castration-resistant prostate cancer. Thus, a complete understanding of the role of truncated AR variants in the progression of castration-resistant prostate cancer may involve evaluating individual tumors for splicing alterations using an unbiased detection method, rather than a targeted approach that focuses on known AR variants.

Regardless of whether one uses a targeted approach (e.g., detecting the deletions or duplications described above) or an unbiased approach (as described immediately above), when one identifies a sample as exhibiting expression of an AR variant, the subject from whom the sample was obtained may be at risk for castration-resistant prostate cancer. As used herein, an individual is considered “at risk” for castration-resistant prostate cancer if the individual exhibits androgen receptor isoform expression indicative of castration-resistant prostate cancer regardless of whether the individual exhibits any symptoms or clinical signs of castration-resistant prostate cancer. Thus, the method can provide diagnosis of castration-resistant prostate cancer in advance of the individual exhibiting any symptoms of having castration-resistant prostate cancer. Consequently, performing the method allows one to commence treatment for castration-resistant prostate cancer and/or dimish reliance on androgen depletion therapy earlier than if the castration-resistant prostate cancer is detected only once the individual experiences one or more symptoms of castration-resistant prostate cancer.

Thus, in some embodiments, the biological sample may be obtained for a subject who has received treatment for prostate cancer. In such embodiments, the results of performing the method can assist a medical professional in evaluating whether the treatment already provided to the subject is likely to be effective and/or whether the subject is likely to develop resistance to the treatment (e.g., androgen depletion therapy).

In some cases, the method can include initiating or modifying the subject's treatment. For example, if the sample reflects expression of an AR variant, that subject may be at risk of developing resistance to androgen depletion therapy and, consequently, development of castration-resistant prostate cancer. One may therefore elect to modify the subject's treatment to de-emphasize androgen depletion therapy. Exemplary drugs used in connection with androgen depletion therapy include, for example, drugs that target and disrupt the androgen:AR interaction through the AR ligand binding domain. Such drugs include, for example, gonadotropin-releasing hormone (GnRH) agonists (e.g., leuprolide), AR antagonists (e.g., bicalutamide, enzalutamide, etc.) or androgen synthesis inhibitors (e.g., abiraterone acetate). In some cases, the treatment modification can include administering to the subject at least one pharmaceutical composition effective for treating castration-resistant prostate cancer. Exemplary drugs used in connection with therapies for treating castration-resistant prostate cancer includes, for example, drugs that target AR-independent pathways and/or drugs that target AR activity through mechanisms that are independent of the androgen; AR interaction through the AR ligand binding domain. Such drugs include, for example, radium-233 dichloride, immunotherapies (sipuleucel-T, ipilimumab, PROSTVAC (Bavarian Nordic Inc., Mountain View, Calif.), taxanes (e.g., docetaxel, cabazitaxel, etc.), kinase inhibitors (e.g., cabozantanib), AR amino-terminal domain inhibitors (e.g., EPI-001, EPI-002, etc.), or AR DNA binding domain inhibitors (e.g., pyrvinium).

In another aspect, this disclosure describes a method for treating castration-resistant prostate cancer. The method generally includes administering to a subject at risk of developing castration-resistant prostate cancer a composition that includes an inhibitor of an androgen receptor (AR) splice variant associated with castration-resistant prostate cancer. As used herein, “an AR splice variant associated with castration-resistant prostate cancer” refers to AR splice variants that correlate with castration-resistant prostate cancer to a statically significant degree in at least one subject or cell line. Exemplary AR splice variants that are associated with castration-resistant prostate cancer include, for example, AR 1/2/2b, AR 1/2/3/2b, AR 1/2/3/CE1, AR 1/2/3/CE2, AR 1/2/3/CE3, or Arv567es.

In some embodiments, the inhibitor can include a polynucleotide that hybridizes to at least a portion of a transcript of the AR splice variant. Suitable inhibitory polynucleotides include, for example, an siRNA (including, e.g., an EsiRNA, a natsiRNA), a microRNA, an antisense RNA, an antisense ssDNA, a peptide nucleic acid (PNA), a morpholino, a locked nucleic acid (LNA), a glycol nucleic acid (GNA), or a threose nucleic acid (TNA). In particular embodiments, the inhibitory polynucleotide can include an siRNA that hybridizes to at least a portion of the splice variant transcript.

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Prostate Cancer Tissues

Genomic DNA samples from the LuCaP series of prostate cancer xenografts and de-identified clinical castration-resistant prostate cancer tissue were obtained from the University of Washington Prostate Cancer Biorepository, and has been described in previous publications (Sun et al., 2010 J Clin Invest 120:2715-30; Corey et al., 2003 Prostate 55:239-46; Roudier et al., 2003 Hum Pathol 34:646-53). De-identified prostatectomy tissue samples were obtained under the direction of the University of Minnesota BioNet tissue resource. One millimeter cores of prostate cancer tissue were obtained from archival formalin-fixed, paraffin-embedded (FFPE) prostatectomy blocks using a tissue microarrayer (Beecher Instruments, Sun Prairie, Wis.) and genomic DNA was isolated using a RecoverAll kit (Applied Biosystems/Ambion, Austin, Tex.).

### Cell Culture

The 22Rv1 (#CRL-2505), LNCaP (#CRL-1740), and VCaP (#CRL-2876) cell lines were obtained from ATCC and cultured according to ATCC protocol. CWR22Pc cells (Dagvadorj et al., 2008 Clin Cancer Res 14:6062-72) were generously provided by Dr. Marja Nevalainen (Thomas Jefferson University) and cultured in RPMI 1640 supplemented with 10% FBS, 2.5 mM L-glutamine, and 0.8 nM dihydrotestosterone (Sigma-Aldrich, St. Louis, Mo.). CWR-R1 cells (Gregory et al., 2001 Cancer Res 61:2892-8) were a kind gift from Dr. Elizabeth Wilson (UNC Chapel Hill) and cultured in RPMI 1640+10% FBS. For androgen response experiments, cells were cultured in RPMI 1640+10% steroid-depleted, charcoal stripped serum (CSS) for 48 hours, treated at t=0 with 1 nM DHT (Sigma-Aldrich, St. Louis, Mo.) or vehicle (EtOH), and then harvested at indicated time points. For long-term culture experiments, CWR-R1 cells were cultured in RPMI 1640+10% CSS. Cells were trypsinized and re-seeded in the appropriate medium when flasks attained 80% confluence.

### Transient Transfections

The CWR-R1 cell line was electroporated with siRNAs targeted to AR exon 7 (target sequence: 5′-GGAACUCGAUCGUAUCAUU; SEQ ID NO:1) or AR exon 1 (5′-CAAGGGAGGUUACACCAAA; SEQ ID NO:2) and/or an MMTV-LUC reporter as described (Dehm et al., 2008 Cancer Res 68:5469-77). Growth of electroporated cells was monitored by crystal violet staining as described (Li et al., 2011 Cancer Res 71:2108-17). Luciferase activity was measured as described (Dehm et al., 2008 Cancer Res 68:5469-77).

### Quantitative Real-Time RT-PCR.

RNA isolation and absolute quantification RT-PCR analysis of alternatively-spliced AR mRNA isoforms was performed as described (Li et al., 2011 Cancer Res 71:2108-17). To correct for different levels of wild-type AR mRNA expression among the prostate cancer cell lines, copy numbers of AR mRNA isoforms were scaled relative to wild-type AR mRNA copy number in each cell line (set to 1). For relative quantification RT-PCR, fold change in expression levels were determined by the comparative Ct method using the equation 2^{-ΔΔCt}.

Genomic PCR.

Genomic PCR was performed as described (Li et al., 2011 Cancer Res 71:2108-17) using primer pairs listed in Table 1.

TABLE 1
Genomic PCR Primer Sequences.

| | | | SEQ |
|---|---|---|---|
| primer | | | ID |
| name | primer use | primer sequence (5′ → 3′) | NO: |
| ExCE3-RV | copy number PCR | CAA CCC CAA CGT CAA AGT CT | 3 |
| Ex4-FW | copy number PCR | CTG TGA CCA GGG AGA ATG GT | 4 |
| Ex4-RV | copy number PCR | TTC AGA TTA CCA AGT TTC TTC AGC | 5 |
| Int1-F1 | deletion-spanning PCR | GCA AAT TGG AGG CAG AAA TC | 6 |
| Int1-F2 | deletion-spanning PCR | CCC AGC TGG TTT AGG AAT CA | 7 |
| Int1-R1 | deletion-spanning PCR | TAT GAA GGA GAA GGG CCA GA | 8 |
| Int1-R2 | deletion-spanning PCR | ATG GCC TTT TGG TTT GAA TG | 9 |
| Int4-F1 | deletion-spanning PCR | CGG AAG CTG AAG AAA CTT GG | 10 |
| Int4-R1 | deletion-spanning PCR | TGG GTG TGG AAA TAG ATG GG | 11 |
| Int4-F2 | deletion-spanning PCR | GCA GCA AAG ATT TCC AAA CTG G | 12 |
| Int4-R2 | deletion-spanning PCR | CCT CTG ATT TTT GGT CTT TCA GCC | 13 |

### Western Blot

Western blotting with AR NTD (N-20, Santa Cruz Biotechnology, Inc., Dallas, Tex.), AR CTD (Santa Cruz C-19, Santa Cruz Biotechnology, Inc., Dallas, Tex.), ERK-2 (Santa Cruz D-2, Santa Cruz Biotechnology, Inc., Dallas, Tex.), and ARV-7 (#AG10008, Precision Antibody, Columbia, Md.) antibodies was performed as described (Li et al., 2011 Cancer Res 71:2108-17).

### Multiplex Ligation-Dependent Probe Assay

MLPA for AR coding sequence was performed using a commercially available kit (P074, MRC Holland) as per the manufacturer's protocol. Briefly, 100 ng of genomic DNA was hybridized at 60° C. for 18 hours with MLPA probe mix. Hybridized probes were ligated and amplified by PCR with labeled universal primers provided with the MLPA kit. PCR reactions were diluted 1:10 in formamide containing ROX-500 size standards (Applied Biosystems, Carlsbad, Calif.), denatured, and resolved by capillary electrophoresis using a Genetic Analyzer 3130XL (Applied Biosystems). Electropherogram peak areas were obtained using PeakScanner software (Applied Biosystems). Peak areas for samples and calibration control (HPV-7 prostate epithelial cell genomic DNA) were block-normalized using X-chromosome p-arm controls and then normalized to HPV-7 copy number with the inference that the HPV-7 genome contains 1 copy of the AR gene. MLPA for AR intron sequences was performed using the exact same protocol with a commercially available reagent kit (EK1, MRC Holland, Amsterdam) and custom-designed oligonucleotide probes (Table 2). Probe pairs that each displayed copy number values outside of one standard deviation from the mean copy number of all AR locus probes from two independent experiments were determined to have increased or decreased copy number at that location.

TABLE 2
MLPA Probe Sequences.

| | | SEQ |
|---|---|---|
| | | ID |
| Probe Name | Sequence 5′ → 3′ | NO: |
| In 1.1 A | GGGTTCCCTAAGGGTTGGAtctggacccgtgatggCCGTGAATTATTGCTTGCACACTCATGGGTG | 14 |
| Probe LPO | | |
| In 1.1 A | ATGCTACTCCCTCTCTCTCATGGCAATTCTTcattctctggttttcgTCTAGATTGGATCTTGCTGGCAC | 15 |
| Probe RPO | | |
| In 1.1 B | GGGTTCCCTAAGGGTTGGAtcggcgttAGGATTTCCCTGGGAATGGTGAGCCTCCATT | 16 |
| Probe LPO | | |
| In 1.1 B | GATGGTTTCAACACACAGCCAAGGCCCTATCttccggaaTCTAGATTGGATCTTGCTGGCAC | 17 |
| Probe RPO | | |
| In 1.2 A | GGGTTCCCTAAGGGTTGGAtaaaaaactaccgtGGTTTGGGGTTAAACCGTGAGTAACCTTATT | 18 |
| Probe LPO | | |
| In 1.2 A | TTCTAGGTCTCAGCCAACTTTGAAGGGCATGgaaaagtcggtggaTCTAGATTGGATCTTGCTGGCAC | 19 |
| Probe RPO | | |
| In 1.2 B | GGGTTCCCTAAGGGTTGGAgcaggaagtcgttaccacctggcccacggagccaatttctcatgct | 20 |
| Probe LPO | TGGTCACAACGTTTGGAGATAGGGAAGAGTT | |
| In 1.2 B | TGTGGATGGATCATGGCAGTGCATGGACAGTgaaaacgtggtgtaccggctgtctggtatgtatgagttt | 21 |
| Probe RPO | gtggtgaTCTAGATTGGATCTTGCTGGCAC | |
| In 1.3 A | GGGTTCCCTAAGGGTTGGAtaggccGAGCTGTCCTACGAGTGTCCAGAATCCTCTG | 22 |
| Probe LPO | | |
| In 1.3 A | TAGTCTTGGGCCTGGTGCTTGAGAGACCCAAagcgagTCTAGATTGGATCTTGCTGGCAC | 23 |
| Probe RPO | | |
| In 1.3 B | GGGTTCCCTAAGGGTTGGAtgtgaatgggGAGTGACCTGTCTTGAATTCAGAACTGCGCA | 24 |
| Probe LPO | | |
| In 1.3 B | GATCATTCCCCATTCTAAGGCCCTCTCATGCcggatgctaaTCTAGATTGGATCTTGCTGGCAC | 25 |
| Probe RPO | | |
| In 1.4 A | GGGTTCCCTAAGGGTTGGAtcagcgcaacacAACCTGGTGATCTCAGCTGGGTGCCAAGGTT | 26 |
| Probe LPO | | |
| In 1.4 A | TCCTAAGCCCAAGTTCCCCATGGTTGAGCCTccttatctggttTCTAGATTGGATCTTGCTGGCAC | 27 |
| Probe RPO | | |
| In 1.4 B | GGGTTCCCTAAGGGTTGGAtgtgggaggcgaaaattggcAGGTCAGGATCAGTGGGAGTGCTACCCAAAA | 28 |
| Probe LPO | | |
| In 1.4 B | TATTTTGCTAGCTGGGGAGTCAGGGAGAAGCgaacgtccggatgctgaagtTCTAGATTGGATCTTGCTG | 29 |
| Probe RPO | GCAC | |
| In 2.1 A | GGGTTCCCTAAGGGTTGGAggtgacgagtgcggttatagcggtccggctgtcgcggatgaatatgaccaG | 30 |
| Probe LPO | GCCCTCTACCTGAAGATATCTTGCTACTGA | |
| In 2.1 A | TGCTGTCTCACAGTGTCTGAAACTCCCATCAgccaacgtccgatatcacgaaggataaatgcagcaaatg | 31 |
| Probe RPO | cctgagcggttTCTAGATTGGATCTTGCTGGCAC | |
| In 2.1 B | GGGTTCCCTAAGGGTTGGAtgcaggtcgaaaaatgggtggatggcACGAGGGGTTTGGAAATCAGAAAAC | 32 |
| Probe LPO | CAGCAG | |
| In 2.1 B | AGGCAGGAAAACTCAGGGCAGCATGGGAGATaggaaagcaatactctgggacacgtaTCTAGATTGGATC | 33 |
| Probe RPO | TTGCTGGCAC | |
| In 2.2 A | GGGTTCCCTAAGGGTTGGAtgTCCACAAGGCCATATGCTTCCTAGACAAAGA | 34 |
| Probe LPO | | |
| In 2.2 A | GAAAAGATTTCTGCCACACTCAGAACGCTTTacTCTAGATTGGATCTTGCTGGCAC | 35 |
| Probe RPO | | |
| In 2.2 B | GGGTTCCCTAAGGGTTGGAtattacgcccgtgccttatccggagaggTAGGAGAAGTGACTTGGAAGCAG | 36 |
| Probe LPO | TCTTGAGA | |
| In 2.2 B | GATTGCCTGTTCCATCCCCTATCTTTGTCCTatgaatgacgcgacaggaagaacttgacTCTAGATTGGA | 37 |
| Probe RPO | TCTTGCTGGCAC | |
| In 3 A | GGGTTCCCTAAGGGTTGGAgagcggaaagagcattattcagcgcccgttccTGCTCCTCAACACAGACTT | 38 |
| Probe LPO | TGACGTTGGGGT | |
| In 3 A | TGGGGGCTACTCTCTTGATTGCTGACTCCCTtgaccgtgtggcttacctgaccgccggtatcgTCTAGAT | 39 |
| Probe RPO | TGGATCTTGCTGGCAC | |
| In 3 B | GGGTTCCCTAAGGGTTGGAgtatggtggccagcggctatgactaccggcgcgacgatgatgcgTGGAGAC | 40 |
| Probe LPO | TTGGGGGAAAGAATCAAGGAGCCT | |
| In 3 B | TCTTGCCTGGGGGAATTTGGCATGCACTTATggcttgtggagttcagccgatctgacttatgtcattacc | 41 |
| Probe RPO | tatgaTCTAGATTGGATCTTGCTGGCAC | |
| In 4 A | GGGTTCCCTAAGGGTTGGAtgccacgacgatgaacagacgcAAGCCATTGAGCCAGGTGTAGTGTGTGCT | 42 |
| Probe LPO | GG | |
| In 4 A | ACACGACAACAACCAGCCCGACTCCTTTGCAtgctgcgtgtggatgaggccatTCTAGATTGGATCTTGC | 43 |
| Probe RPO | TGGCAC | |
| In 4 B | GGGTTCCCTAAGGGTTGGAgtccaagggaccgagtgaaagtgtggatgcagccctACCAGTTCAGCCTCT | 44 |
| Probe LPO | TTCTCTCTATCCCAGG | |
| In 4 B | GAAGCCCTAGGTCACTCTTGCAAAATCTTAGgttgcccaactttacccgtggcaatgcccgcgcacaTCT | 45 |
| Probe RPO | AGATTGGATCTTGCTGGCAC | |
| Xp22 A | GGGTTCCCTAAGGGTTGGAtcaaGCTGAGCACCTCCTCAAAATAGACCCTCAGT | 46 |
| Probe LPO | | |
| Xp22 A | TATAGCCAGATGCATCTCGTGAGCCAGGATGaaagTCTAGATTGGATCTTGCTGGCAC | 47 |
| Probe RPO | | |
| Xp22 B | GGGTTCCCTAAGGGTTGGAgacattgttacactgtggaggagtccatgacgaaagatgaACAAAGTCAGC | 48 |
| Probe LPO | AGGGCAAACTCCACTGTATC | |
| Xp22 B | TGGTAGAGAATCCAGAAGCCAGGCCAAGTCTactgattgcccgtctccgctcgctgggtgaacaactgaa | 49 |
| Probe RPO | cTCTAGATTGGATCTTGCTGGCAC | |
| Xp21 A | GGGTTCCCTAAGGGTTGGAtcggtgagacgtgggaggcgaaaattggcgCACCCCCAAACATATGGGCAT | 50 |
| Probe LPO | TACCATTCCA | |
| Xp21 A | TCACCCCCTGGAGTGCCAACTATTTGACTGAaacgtccggatgctgaagtgatggcagagcTCTAGATTG | 51 |
| Probe RPO | GATCTTGCTGGCAC | |
| Xp21 B | GGGTTCCCTAAGGGTTGGATCCCATTGGAAATCAAGCTGGGAGAGAGCTT | 52 |
| Probe LPO | | |
| Xp21 B | CCTGTAGCTTCACCCTTTCCACAGGCGTTGCTCTAGATTGGATCTTGCTGGCAC | 53 |
| Probe RPO | | |
| Xp11 LPO | GGGTTCCCTAAGGGTTGGAgactcccagctggaccgctacgaaatgcgcgtatATCTTTGTTCCTCCCAA | 54 |
| | CACCAATGCCCCAC | |
| Xp11 RPO | TCCAAGTTCCCATTTCCCACCTCTTGGATGCggggatgggggccgggtgaggaaagctggctgatTCTAG | 55 |
| | ATTGGATCTTGCTGGCAC | |
| Xq11 LPO | GGGTTCCCTAAGGGTTGGAgatgcgtgatggtggcctgttctccggaggtggacgatgaagaccttcAGT | 56 |
| | GAGACCTGAACATCAGAGGGGGACTGAG | |
| Xq11 RPO | TAGTAAGAGTAGCTAGGAGGCCACAGACTGCcgctggaaagtgaaacccggtatggatgtggcttcggtc | 57 |
| | ccttctgtaTCTAGATTGGATCTTGCTGGCAC | |
| Xq26 LPO | GGGTTCCCTAAGGGTTGGAgtgagcagtcaggtggcgtgatacgtggtgtttttgatgaccGCAAGCTCT | 58 |
| | TCAGCCTCCCTTTTCTCCCATA | |
| Xq26 RPO | TTCCAAAAGTTGCCTTTGGGTTCGTCCACTActgaaaatatcagctatgccggacagggcgtgcgcgttg | 59 |
| | aagTCTAGATTGGATCTTGCTGGCAC | |
| Xq28 A | GGGTTCCCTAAGGGTTGGAtcatccggtgaagagattAGGAGACGACACTGTAGTTTTCACCCGCTGT | 60 |
| Probe LPO | | |
| Xq28 A | GGCTGAGATGTTGCCAAAATCTGAGATCCCTgagccacctgacagtgtgTCTAGATTGGATCTTGCTGGC | 61 |
| Probe RPO | AC | |
| Xq28 B | GGGTTCCCTAAGGGTTGGAtctgacctttcacatctggacagcCACCAACTCCAAAACCGTGGGATTCTG | 62 |
| Probe LPO | CTTT | |
| Xq28 B | TCATCCTCTGTGGTGTTGGCCAGATCATACTgtacagcccgttcagcacctgggtTCTAGATTGGATCTT | 63 |
| Probe RPO | GCTGGCAC | |

### Paired-End Library Creation, Sequence Capture, and Next-Generation Sequencing

Genomic DNA from CWR22Pc, 22Rv1, and CWR-R1 cells was fragmented using an S220 ultra-sonicator (Covaris, Woburn, Mass.) with Agilent SureSelect parameters. A Bioanalyzer DNA 1000 chip (Agilent, Santa Clara, Calif.) was used to verify DNA samples sheared with fragment peaks between 150 bp-200 bp. Paired-end sequencing libraries were generated from sheared DNA samples using a SureSelect Library Preparation Kit (Agilent) and amplified for sequence capture as per the manufacturer's protocol. The amplified DNA libraries were hybridized and captured using overlapping, tiled SureSelect baits (Agilent) custom-designed to provide 2× coverage of non-repetitive regions of the AR locus (Table 3). Target-enriched libraries were amplified for 16-cycles to add index tags and generate sufficient template for flowcell clustering. Final libraries were quantified via quantitative PCR (Kapa Biosystems, Woburn, Mass.), normalized, and pooled prior to clustering on a single lane of a flowcell. The flowcell was loaded on a Genome Analyzer IIx (GAIIx, Illumina, San Diego, Calif.) for paired-end sequencing at 76 cycles (2×76 bp). Data analysis methodology is provided below under the heading “Analysis of Paired-End Sequence Data.”

TABLE 3
SureSelect Bait Library for AR Sequence Capture

| TargetID | Bait Location | Sequence (5′ → 3′) | SEQ ID NO: |
|---|---|---|---|
| chrX: 66763874- | chrX: 66763963- | AGCTAGCCGCTCCAGTGCTGTACAGGAGCCGAAGGGACGCACCACGCCAGCCCCAGCCCGGCTCCAGCGACAGCCAACGCCTCTTGCAGC | 64 |
| 66944119 | 66764082 | GCGGCGGCTTCGAAGCCGCCGCCCGGAGCT | |
| chrX: 66763874- | chrX: 66763843- | GCATTTGCTCTCCACCTCCCAGCGCCCCCTCCGAGATCCCGGGGAGCCAGCTTGCTGGGAGAGCGGGACGGTCCGGAGCAAGCCCAGAGG | 65 |
| 66944119 | 66763962 | CAGAGGAGGCGACAGAGGGAAAAAGGGCCG | |
| chrX: 66763874- | chrX: 66764023- | GCTCCAGCGACAGCCAACGCCTCTTGCAGCGCGGCGGCTTCGAAGCCGCCGCCCGGAGCTGCCCTTTCCTCTTCGGTGAAGTTTTTAAAA | 66 |
| 66944119 | 66764142 | GCTGCTAAAGACTCGGAGGAAGCAAGGAAA | |
| chrX: 66763874- | chrX: 66763903- | GAGCGGGACGGTCCGGAGCAAGCCCAGAGGCAGAGGAGGCGACAGAGGGAAAAAGGGCCGAGCTAGCCGCTCCAGTGCTGTACAGGAGCC | 67 |
| 66944119 | 66764022 | GAAGGGACGCACCACGCCAGCCCCAGCCCG | |
| chrX: 66763874- | chrX: 66764444- | ACCCGCCTGGTTAGGCTGCACGCGGAGAGAACCCTCTGTTTTCCCCCACTCTCTCTCCACCTCCTCCTGCCTTCCCCACCCCGAGTGCGG | 68 |
| 66944119 | 66764563 | AGCCAGAGATCAAAAGATGAAAAGGCAGTC | |
| chrX: 66763874- | chrX: 66764384- | TTGCAAAGAAGGCTCTTAGGAGCCAGGCGACTGGGGAGCGGCTTCAGCACTGCAGCCACGACCCGCCTGGTTAGGCTGCACGCGGAGAGA | 69 |
| 66944119 | 66764503 | ACCCTCTGTTTTCCCCCACTCTCTCTCCAC | |
| chrX: 66763874- | chrX: 66764324- | CAGCCCGAGTTTGCAGAGAGGTAACTCCCTTTGGCTGCGAGCGGGCGAGCTAGCTGCACATTGCAAAGAAGGCTCTTAGGAGCCAGGCGA | 70 |
| 66944119 | 66764443 | CTGGGGAGCGGCTTCAGCACTGCAGCCACG | |
| chrX: 66763874- | chrX: 66765011- | GAAGGGTCTACCCTCGGCCGCCGTCCAAGACCTACCGAGGAGCTTTCCAGAATCTGTTCCAGAGCGTGCGCGAAGTGATCCAGAACCCGG | 71 |
| 66944119 | 66765130 | GCCCCAGGCACCCAGAGGCCGCGAGCGCAG | |
| chrX: 66763874- | chrX: 66764951- | GTAAGGGAAGTAGGTGGAAGATTCAGCCAAGCTCAAGGATGGAAGTGCAGTTAGGGCTGGGAAGGGTCTACCCTCGGCCGCCGTCCAAGA | 72 |
| 66944119 | 66765070 | CCTACCGAGGAGCTTTCCAGAATCTGTTCC | |
| chrX: 66763874- | chrX: 66764771- | GATCTTGTCCACCGTGTGTCTTCTTCTGCACGAGACTTTGAGGCTGTCAGAGCGCTTTTTGCGTGGTTGCTCCCGCAAGTTTCCTTCTCT | 73 |
| 66944119 | 66764890 | GGAGCTTCCCGCAGGTGGGCAGCTAGCTGC | |
| chrX: 66763874- | chrX: 66764891- | AGCGACTACCGCATCATCACAGCCTGTTGAACTCTTCTGAGCAAGAGAAGGGGAGGCGGGGTAAGGGAAGTAGGTGGAAGATTCAGCCAA | 74 |
| 66944119 | 66765010 | GCTCAAGGATGGAAGTGCAGTTAGGGCTGG | |
| chrX: 66763874- | chrX: 66764831- | GCGTGGTTGCTCCCGCAAGTTTCCTTCTCTGGAGCTTCCCGCAGGTGGGCAGCTAGCTGCAGCGACTACCGCATCATCACAGCCTGTTGA | 75 |
| 66944119 | 66764950 | ACTCTTCTGAGCAAGAGAAGGGGAGGCGGG | |
| chrX: 66763874- | chrX: 66764651- | CTACTTCAGTGGACACTGAATTTGGAAGGTGGAGGATTTTGTTTTTTTCTTTTAAGATCTGGGCATCTTTTGAATCTACCCTTCAAGTAT | 76 |
| 66944119 | 66764770 | TAAGAGACAGACTGTGAGCCTAGCAGGGCA | |
| chrX: 66763874- | chrX: 66764711- | GGGCATCTTTTGAATCTACCCTTCAAGTATTAAGAGACAGACTGTGAGCCTAGCAGGGCAGATCTTGTCCACCGTGTGTCTTCTTCTGCA | 77 |
| 66944119 | 66764830 | CGAGACTTTGAGGCTGTCAGAGCGCTTTTT | |
| chrX: 66763874- | chrX: 66765500- | TCCCCGGCTTAAGCAGCTGCTCCGCTGACCTTAAAGACATCCTGAGCGAGGCCAGCACCATGCAACTCCTTCAGCAACAGCAGCAGGAAG | 78 |
| 66944119 | 66765619 | CAGTATCCGAAGGCAGCAGCAGCGGGAGAG | |
| chrX: 66763874- | chrX: 66766220- | TGGCGAGCCTGCATGGCGCGGGTGCAGCGGGACCCGGTTCTGGGTCACCCTCAGCCGCCGCTTCCTCATCCTGGCACACTCTCTTCACAG | 79 |
| 66944119 | 66766339 | CCGAAGAAGGCCAGTTGTATGGACCGTGTg | |
| chrX: 66763874- | chrX: 66765920- | CTTTCAAGGGAGGTTACACCAAAGGGCTAGAAGGCGAGAGCCTAGGCTGCTCTGGCAGCGCTGCAGCAGGGAGCTCCGGGACACTTGAAC | 80 |
| 66944119 | 66766039 | TGCCGTCTACCCTGTCTCTCTACAAGTCCG | |
| chrX: 66763874- | chrX: 66765680- | CCATTTCTGACAACGCCAAGGAGTTGTGTAAGGCAGTGTCGGTGTCCATGGGCCTGGGTGTGGAGGCGTTGGAGCATCTGAGTCCAGGGG | 81 |
| 66944119 | 66765799 | AACAGCTTCGGGGGGATTGCATGTACGCCC | |
| chrX: 66763874- | chrX: 66765980- | CTGCAGCAGGGAGCTCCGGGACACTTGAACTGCCGTCTACCCTGTCTCTCTACAAGTCCGGAGCACTGGACGAGGCAGCTGCGTACCAGA | 82 |
| 66944119 | 66766099 | GTCGCGACTACTACAACTTTCCACTGGCTC | |
| chrX: 66763874- | chrX: 66766160- | ACCCGCTGGACTACGGCAGCGCCTGGGCGGCTGCGGCGGCGCAGTGCCGCTATGGGGACCTGGCGAGCCTGCATGGCGCGGGTGCAGCGG | 83 |
| 66944119 | 66766279 | GACCCGGTTCTGGGTCACCCTCAGCCGCCG | |
| chrX: 66763874- | chrX: 66765860- | AAGGTTCTCTGCTAGACGACAGCGCAGGCAAGAGCACTGAAGATACTGCTGAGTATTCCCCTTTCAAGGGAGGTTACACCAAAGGGCTAG | 84 |
| 66944119 | 66765979 | AAGGCGAGAGCCTAGGCTGCTCTGGCAGCG | |
| chrX: 66763874- | chrX: 66765800- | CACTTTTGGGAGTTCCACCCGCTGTGCGTCCCACTCCTTGTGCCCCATTGGCCGAATGCAAAGGTTCTCTGCTAGACGACAGCGCAGGCA | 85 |
| 66944119 | 66765919 | AGAGCACTGAAGATACTGCTGAGTATTCCC | |
| chrX: 66763874- | chrX: 66765440- | CACCTCCGGACGAGGATGACTCAGCTGCCCCATCCACGTTGTCCCTGCTGGGCCCCACTTTCCCCGGCTTAAGCAGCTGCTCCGCTGACC | 86 |
| 66944119 | 66765559 | TTAAAGACATCCTGAGCGAGGCCAGCACCA | |
| chrX: 66763874- | chrX: 66765260- | agGGTGAGGATGGTTCTCCCCAAGCCCATCGTAGAGGCCCCACAGGCTACCTGGTCCTGGATGAGGAACAGCAACCTTCACAGCCGCAGT | 87 |
| 66944119 | 66765379 | CGGCCCTGGAGTGCCACCCCGAGAGAGGTT | |
| chrX: 66763874- | chrX: 66765740- | TGGAGGCGTTGGAGCATCTGAGTCCAGGGGAACAGCTTCGGGGGGATTGCATGTACGCCCCACTTTTGGGAGTTCCACCCGCTGTGCGTC | 88 |
| 66944119 | 66765859 | CCACTCCTTGTGCCCCATTGGCCGAATGCA | |
| chrX: 66763874- | chrX: 66765560- | TGCAACTCCTTCAGCAACAGCAGCAGGAAGCAGTATCCGAAGGCAGCAGCAGCGGGAGAGCGAGGGAGGCCTCGGGGGCTCCCACTTCCT | 89 |
| 66944119 | 66765679 | CCAAGGACAATTACTTAGGGGGCACTTCGA | |
| chrX: 66763874- | chrX: 66765620- | CGAGGGAGGCCTCGGGGGCTCCCACTTCCTCCAAGGACAATTACTTAGGGGGCACTTCGACCATTTCTGACAACGCCAAGGAGTTGTGTA | 90 |
| 66944119 | 66765739 | AGGCAGTGTCGGTGTCCATGGGCCTGGGTG | |
| chrX: 66763874- | chrX: 66765320- | ATGAGGAACAGCAACCTTCACAGCCGCAGTCGGCCCTGGAGTGCCACCCCGAGAGAGGTTGCGTCCCAGAGCCTGGAGCCGCCGTGGCCG | 91 |
| 66944119 | 66765439 | CCAGCAAGGGGCTGCCGCAGCAGCTGCCAG | |
| chrX: 66763874- | chrX: 66766100- | TGGCCGGACCGCCGCCCCCTCCGCCGCCTCCCCATCCCCACGCTCGCATCAAGCTGGAGAACCCGCTGGACTACGGCAGCGCCTGGGCGG | 92 |
| 66944119 | 66766219 | CTGCGGCGGCGCAGTGCCGCTATGGGGACC | |
| chrX: 66763874- | chrX: 66765380- | GCGTCCCAGAGCCTGGAGCCGCCGTGGCCGCCAGCAAGGGGCTGCCGCAGCAGCTGCCAGCACCTCCGGACGAGGATGACTCAGCTGCCC | 93 |
| 66944119 | 66765499 | CATCCACGTTGTCCCTGCTGGGCCCCACTT | |
| chrX: 66763874- | chrX: 66766040- | GAGCACTGGACGAGGCAGCTGCGTACCAGAGTCGCGACTACTACAACTTTCCACTGGCTCTGGCCGGACCGCCGCCCCCTCCGCCGCCTC | 94 |
| 66944119 | 66766159 | CCCATCCCCACGCTCGCATCAAGCTGGAGA | |
| chrX: 66763874- | chrX: 66770066- | GCTTCCAATTGCAGGGTCATTTTGGGGTGCTTTCTTGCCTGTACTAATTTTATCTCATCAAGCTTCCATTCTTTGAGCTGTAAACTTTGA | 95 |
| 66944119 | 66770185 | AATAATATACTGGATTTGCTGGTACGTTTA | |
| chrX: 66763874- | chrX: 66769766- | CTATCTTACAAGCAACAGTTTGTCTTGTAAAGCAGAATTTTCCTTTGAAACCAAGACAGATTATTTCTGCCCATAGGCTTCAGGAACCAA | 96 |
| 66944119 | 66769885 | TATTTTGGCAAGAAGCATCTTTTCTTTGTG | |
| chrX: 66763874- | chrX: 66768746- | GTGATTGCTGCACTGAATATGAGGAGTCTAGTTAAAGGGACAACTGGTGTTCCTGTCTTGTGAGTTGACGAAGACTTTCCATTTCTAGGA | 97 |
| 66944119 | 66768865 | TATAGAAAATCCTTAAGCCGGTTTATTGAA | |
| chrX: 66763874- | chrX: 66767246- | AGAGTAGGCAGAGGGGCAACTTTCTTGGTAAAGACTTCACAGGATTTGCACTCACAGTTTCTCAACGTTGGTTGACTATGTTGAAAGTAG | 98 |
| 66944119 | 66767365 | TTGCTTGGGTCGGTTTTCTCTTGTAAAGTG | |
| chrX: 66763874- | chrX: 66769946- | AGCCACACTGGGTGGCACCCCATTCGGAAGTATACACAGGAAGTAGCCCTCTTGCTTGTTCACAGCTCAAGTCAGCCAAAGATTAACACT | 99 |
| 66944119 | 66770065 | GGTGAGAGATATTTTCAAAGAAGTTTGCAG | |
| chrX: 66763874- | chrX: 66766406- | gcgAGGCGGGAGCTGTAGCCCCCTACGGCTACACTCGGCCCCCTCAGGGGCTGGCGGGCCAGGAAAGCGACTTCACCGCACCTGATGTGT | 100 |
| 66944119 | 66766525 | GGTACCCTGGCGGCATGGTGAGCAGAGTGC | |
| chrX: 66763874- | chrX: 66768446- | CACAACTTACTTACTTAACAGGGAAAAAACTGATGGTTCCACATATTTGCTAAAAAATGTGTGCCTTCAAAGACAAAACCAAAATTTTTA | 101 |
| 66944119 | 66768565 | GGGAATAACTATAGAGAGCAAAAGTTACTC | |
| chrX: 66763874- | chrX: 66769526- | CTAAGAGAGTGCATGCCAGAATTAGAGTTTGGGGTTTAGAGAAATTATCCAGATGCCAAAAGAACATTTTAATTTTTCTCTTGGTAATTT | 102 |
| 66944119 | 66769645 | GTTCTGGTCTCCATAGTAGGTAGTATTTTA | |
| chrX: 66763874- | chrX: 66767126- | GAAAGTGGTCTCTGGGTGCTGAGGTCTGCTGTGTGAAAGGGTGAACTTCTTTCTCCTGAAGCAACTGGGGACTTGCTCCAGGGCTGGAGG | 103 |
| 66944119 | 66767245 | TCAGTAGAGATAATCCAAACCGTCATGTTT | |
| chrX: 66763874- | chrX: 66767186- | GCAACTGGGGACTTGCTCCAGGGCTGGAGGTCAGTAGAGATAATCCAAACCGTCATGTTTAGAGTAGGCAGAGGGGCAACTTTCTTGGTA | 104 |
| 66944119 | 66767305 | AAGACTTCACAGGATTTGCACTCACAGTTT | |
| chrX: 66763874- | chrX: 66770546- | CAGAGAAGGAAAAGGAAATCTCTCCCTACCCCCCACCTCCACCATTTTCTCTTTGTCTGCAGCTTCCTCAAGTGCTGCCTGTCCCCGATT | 105 |
| 66944119 | 66770665 | TTCTTTTATTCCACTCCTTTCATGTTTTTG | |
| chrX: 66763874- | chrX: 66768326- | GACGGTGGAGCCCCTCTTCTACTGTAAACTTTTCTTGGGGGAAAATGTCTAAGGTGCATTTTGACCTGCCATGATACTAAACCCAGACAC | 106 |
| 66944119 | 66768445 | TGGAACCTTCCATCTTCTGCATGCCTCCCC | |
| chrX: 66763874- | chrX: 66768986- | CTTACTTTCTCTTTTTTTCCCTCATTTTGTAGGGATACAATTTGGTGAAAGGCAAGAGATTTCTTAAGCCAAAGCAAGAGTGTCTTCCCT | 107 |
| 66944119 | 66769105 | CTCTGTGTTGCATGCATTATGTGCCATGTT | |
| chrX: 66763874- | chrX: 66769886- | GTCAGCAAATAGGTGGTGAGTTCTGTCTGGATCCCAACAATCAACACCTGAGGACCAAATAGCCACACTGGGTGGCACCCCATTCGGAAG | 108 |
| 66944119 | 66770005 | TATACACAGGAAGTAGCCCTCTTGCTTGTT | |
| chrX: 66763874- | chrX: 66766466- | AGGAAAGCGACTTCACCGCACCTGATGTGTGGTACCCTGGCGGCATGGTGAGCAGAGTGCCCTATCCCAGTCCCACTTGTGTCAAAAGCG | 109 |
| 66944119 | 66766585 | AAATGGGCCCCTGGATGGATAGCTACTCCG | |
| chrX: 66763874- | chrX: 66766526- | CCTATCCCAGTCCCACTTGTGTCAAAAGCGAAATGGGCCCCTGGATGGATAGCTACTCCGGACCTTACGGGGACATGCGGTAAGTTTTTC | 110 |
| 66944119 | 66766645 | CTTCCAGAAATGTCGCCTTTCGGCCCAGGG | |
| chrX: 66763874- | chrX: 66769106- | TGAGCTAAAAATCTCAAAATTGGGCAGGCTTCCAATGACCTGTTGGGTCCCTCCCTTTACCATTCATGTGTGTGTTTATGTACATAATTT | 111 |
| 66944119 | 66769225 | TGTGGAGGGGTTTTTTTAAACCTTAGTAAC | |
| chrX: 66763874- | chrX: 66766646- | CAGAGTCACTCTGTGTTCTGGGGTATCTAGCGGCTCCTACCTGCGCGAACACTCAGATTGCCCCTGGGAGAGCTCAGCAGGGTAAACCTA | 112 |
| 66944119 | 66766765 | GAGCTCTCCCGTGGACTCCCGGCCTGCCAG | |
| chrX: 66763874- | chrX: 66766586- | GACCTTACGGGGACATGCGGTAAGTTTTTCCTTCCAGAAATGTCGCCTTTCGGCCCAGGGCAGAGTCACTCTGTGTTCTGGGGTATCTAG | 113 |
| 66944119 | 66766705 | CGGCTCCTACCTGCGCGAACACTCAGATTG | |
| chrX: 66763874- | chrX: 66768566- | CCATCAAGTAGACAACGAGCTTGGTGATTTTATTTCAGGTCTTAATGAAAAAAGCTTCTTTATGAGGAAGGTTATCATATCTTGGTGCCT | 114 |
| 66944119 | 66768685 | CCTTGACAGTCCGCTTAAATTAATGACATA | |
| chrX: 66763874- | chrX: 66767306- | CTCAACGTTGGTTGACTATGTTGAAAGTAGTTGCTTGGGTCGGTTTTCTCTTGTAAAGTGTTTATTTTCTCTGTGGATTATAACAGATCC | 115 |
| 66944119 | 66767425 | ACAGCCCCCTACTTCAGGTTTGCATCAGAT | |
| chrX: 66763874- | chrX: 66766826- | CCAGATTCTTCAACTCCCCCAACCGCCCCAAATTCTCACTACCTCCTGGTACTCGAGGTCCCAAACAGAAATCCTATTGCACGGGCCACC | 116 |
| 66944119 | 66766945 | TTCAGAGATAAAGCTCCCAAGCCCTCCACT | |
| chrX: 66763874- | chrX: 66770726- | CATGCTCCTAAAGAATTTCTTTTTTAAAAAAAATCTGTAGAGTAGTAGATTAGATTAACCCCAGTATCTCTCCCTTAAGACTAGATGACA | 117 |
| 66944119 | 66770845 | TGAGGGGATTGCAAAATGAATAGCTGGGtt | |
| chrX: 66763874- | chrX: 66768266- | TTCATTTTATAAGAACAAATTTACTTGGTAGGGTTAATTTTTTTTCTAGGGCTGTCACTAGACGGTGGAGCCCCTCTTCTACTGTAAACT | 118 |
| 66944119 | 66768385 | TTTCTTGGGGGAAAATGTCTAAGGTGCATT | |
| chrX: 66763874- | chrX: 66768146- | TTTAATGTTTGACTTACAAGATTTTCAGAGGGTTCATTTGATATTGTCAAAGTCTTTTCCAGTTAATTTAGACTCTTCATTTTTGTAATG | 119 |
| 66944119 | 66768265 | GGTTTATGCTATGGGACAAAAAAAGTATTC | |
| chrX: 66763874- | chrX: 66770126- | AGCTTCCATTCTTTGAGCTGTAAACTTTGAAATAATATACTGGATTTGCTGGTACGTTTAATTTTCTTTGTTAAGTGTTTTCATTCCCAT | 120 |
| 66944119 | 66770245 | AGTAATTTTTCATCTAGTGTACATATATGC | |
| chrX: 66763874- | chrX: 66769286- | GCAAAGGTGGTCTCTTTTACTTAATTTAGCATGTGGTTTGAACAGAAGGAAAAATAAAAAGTGATGGGGCTTGTGTGCAACCCTGATGAT | 121 |
| 66944119 | 66769405 | ATTTTATGGAGCTGTCTGTCTTCTCTCTGA | |
| chrX: 66763874- | chrX: 66768026- | CTTCTTACAAGGTAGATAGCACAGTGTTAGTAAAGAAAGAAAGAGGAGGGTAGGATTTCATATTATTTCGTGGGCTGTTGAAGAAACAGC | 122 |
| 66944119 | 66768145 | TTCTTACCAGGCTTTACATTCCATTAGGTT | |
| chrX: 66763874- | chrX: 66770306- | CAAGCTACTTGGTGGGATTATGTGAACTGGAGTTAGAAATGTGGACAATTTTATTATGATTATTTTTAATGGTGATATCAAGATCACCAG | 123 |
| 66944119 | 66770425 | TTTCATTCGGAACCTTGCATAAGCAGGGAG | |
| chrX: 66763874- | chrX: 66769466- | TCTTATATTCCAGCAAGCAGCACAATAATATGACAAAAATTTATTCTTGGGAGTTGGGTTCTAAGAGAGTGCATGCCAGAATTAGAGTTT | 124 |
| 66944119 | 66769585 | GGGGTTTAGAGAAATTATCCAGATGCCAAA | |
| chrX: 66763874- | chrX: 66767426- | CTATAAAGAGGAGAATATTCTTTTAATGTACAATTTAATTAGGCTTGACTCTGACTTACAAAACTGTTGGAAAACATTTTTTTGTAAAGC | 125 |
| 66944119 | 66767545 | ATTTCCTGCTATTTCAGTGTGCTCCAAAAT | |
| chrX: 66763874- | chrX: 66769646- | GTAGTGCTTTGATATTGACAAGTCTTGCTCCCTTTCTCTATTAGATTTTTCAAAATAAGGCATTTTATTAATTCCTCTTTCCTTCTCCTC | 126 |
| 66944119 | 66769765 | TCTCCTCTCAGTTATCAAGCATTTTTATGA | |
| chrX: 66763874- | chrX: 66769406- | GATCAAACAGGACTACAACTTTGTTAATTGACCACTGGCTCCCTTGGCAAAAGTAGGGCTTCTTATATTCCAGCAAGCAGCACAATAATA | 127 |
| 66944119 | 66769525 | TGACAAAAATTTATTCTTGGGAGTTGGGTT | |
| chrX: 66763874- | chrX: 66769826- | TTATTTCTGCCCATAGGCTTCAGGAACCAATATTTTGGCAAGAAGCATCTTTTCTTTGTGGTCAGCAAATAGGTGGTGAGTTCTGTCTGG | 128 |
| 66944119 | 66769945 | ATCCCAACAATCAACACCTGAGGACCAAAT | |
| chrX: 66763874- | chrX: 66767906- | TTCTTTGTTTCTATGGCTGTTTTCTTTTACCTGTGACTTCTCCGAAATTTCTTTGTTAGCCTTAACATCTTTGTTTGGGGACTTAAATCC | 129 |
| 66944119 | 66768025 | AGCAATTTGCCTTCTTTCACTGATGCTTTC | |
| chrX: 66763874- | chrX: 66768386- | TTGACCTGCCATGATACTAAACCCAGACACTGGAACCTTCCATCTTCTGCATGCCTCCCCCACAACTTACTTACTTAACAGGGAAAAAAC | 130 |
| 66944119 | 66768505 | TGATGGTTCCACATATTTGCTAAAAAATGT | |
| chrX: 66763874- | chrX: 66766886- | CCAAACAGAAATCCTATTGCACGGGCCACCTTCAGAGATAAAGCTCCCAAGCCCTCCACTCTTCCTTTCCTCCTGTCCTCAAAGTCTGAG | 131 |
| 66944119 | 66767005 | AACCTCAACAGGAATTTGGGCAATTTCTCC | |
| chrX: 66763874- | chrX: 66768626- | TATGAGGAAGGTTATCATATCTTGGTGCCTCCTTGACAGTCCGCTTAAATTAATGACATAAACTAATGAGAATTTAGCAGTTCCTGCAGA | 132 |
| 66944119 | 66768745 | AAGTACAAGTTTATTTTTTTTTTCTGGTTT | |
| chrX: 66763874- | chrX: 66770366- | TATTTTTAATGGTGATATCAAGATCACCAGTTTCATTCGGAACCTTGCATAAGCAGGGAGCAGAATGCGGACTGGGTGTGGCAAAGCAAG | 133 |
| 66944119 | 66770485 | GGCTTATTTTATAGCCAAACCTGAAATCAC | |
| chrX: 66763874- | chrX: 66767966- | CTTAACATCTTTGTTTGGGGACTTAAATCCAGCAATTTGCCTTCTTTCACTGATGCTTTCCTTCTTACAAGGTAGATAGCACAGTGTTAG | 134 |
| 66944119 | 66768085 | TAAAGAAAGAAAGAGGAGGGTAGGATTTCA | |
| chrX: 66763874- | chrX: 66770666- | ACATTGAAATACAGACTCTTCTTTCCACTTCTCAGGGTATTTTTCTTATTACACCTGTGGCATGCTCCTAAAGAATTTCTTTTTTAAAAA | 135 |
| 66944119 | 66770785 | AAATCTGTAGAGTAGTAGATTAGATTAACC | |
| chrX: 66763874- | chrX: 66768206- | AGTTAATTTAGACTCTTCATTTTTGTAATGGGTTTATGCTATGGGACAAAAAAAGTATTCTTCATTTTATAAGAACAAATTTACTTGGTA | 136 |
| 66944119 | 66768325 | GGGTTAATTTTTTTTCTAGGGCTGTCACTA | |
| chrX: 66763874- | chrX: 66769346- | GTGATGGGGCTTGTGTGCAACCCTGATGATATTTTATGGAGCTGTCTGTCTTCTCTCTGAGATCAAACAGGACTACAACTTTGTTAATTG | 137 |
| 66944119 | 66769465 | ACCACTGGCTCCCTTGGCAAAAGTAGGGCT | |
| chrX: 66763874- | chrX: 66768866- | AATTAATCAATTTAATCAGAATGCAATCAATTCCAATACAAAAGTTAGTATTTTCTTTCTTTTTATTGAAAATTAATTTAATCAGAATAC | 138 |
| 66944119 | 66768985 | AATCAATTCCAATCCAAAAGTTGATATTTT | |
| chrX: 66763874- | chrX: 66767486- | AAACTGTTGGAAAACATTTTTTTGTAAAGCATTTCCTGCTATTTCAGTGTGCTCCAAAATCTCCACTGGGGAGGGTGGAGTGAGGTTTTT | 139 |
| 66944119 | 66767605 | TATTATATTCCTTTATTTTTAGGACATGTT | |
| chrX: 66763874- | chrX: 66770426- | CAGAATGCGGACTGGGTGTGGCAAAGCAAGGGCTTATTTTATAGCCAAACCTGAAATCACAACTCTGAAAAATAAAAAAAAAAAAAACCA | 140 |
| 66944119 | 66770545 | AACAAAAAAATCAAGTTTTGTGAGCTTGGT | |
| chrX: 66763874- | chrX: 66767666- | TGAGCCGTAATCTTACCCCAAAGTTTTAATTAGCATATGAGAAAAGTGGCAGGCAATTGCATCGTGCTTATTAAAAATTATTCCTCACCG | 141 |
| 66944119 | 66767785 | CAGTTGTTGAGCTTCTTGGAGACCATGCTG | |
| chrX: 66763874- | chrX: 66767546- | CTCCACTGGGGAGGGTGGAGTGAGGTTTTTTATTATATTCCTTTATTTTTAGGACATGTTTGCATTTTAGAATATGTGCAGTTAGCTCTA | 142 |
| 66944119 | 66767665 | ACAAATTGAGTAAGAACTCTTAATGACCTA | |
| chrX: 66763874- | chrX: 66770006- | CACAGCTCAAGTCAGCCAAAGATTAACACTGGTGAGAGATATTTTCAAAGAAGTTTGCAGGCTTCCAATTGCAGGGTCATTTTGGGGTGC | 143 |
| 66944119 | 66770125 | TTTCTTGCCTGTACTAATTTTATCTCATCA | |
| chrX: 66763874- | chrX: 66770246- | ATTTAAAACAAAAATTCTTTGGTCTCCTTATGCGTATATGCACTGCGGCTTGTACACGTACAAGCTACTTGGTGGGATTATGTGAACTGG | 144 |
| 66944119 | 66770365 | AGTTAGAAATGTGGACAATTTTATTATGAT | |
| chrX: 66763874- | chrX: 66768926- | TTTTATTGAAAATTAATTTAATCAGAATACAATCAATTCCAATCCAAAAGTTGATATTTTCTTACTTTCTCTTTTTTTCCCTCATTTTGT | 145 |
| 66944119 | 66769045 | AGGGATACAATTTGGTGAAAGGCAAGAGAT | |
| chrX: 66763874- | chrX: 66768506- | GTGCCTTCAAAGACAAAACCAAAATTTTTAGGGAATAACTATAGAGAGCAAAAGTTACTCCCATCAAGTAGACAACGAGCTTGGTGATTT | 146 |
| 66944119 | 66768625 | TATTTCAGGTCTTAATGAAAAAAGCTTCTT | |
| chrX: 66763874- | chrX: 66766766- | AGGTTTAACCTGAGCTCTCCTAATTTCTGCTGCGTGCCCTGGGTGCTGATTCCTGCCCTCCCAGATTCTTCAACTCCCCCAACCGCCCCA | 147 |
| 66944119 | 66766885 | AATTCTCACTACCTCCTGGTACTCGAGGTC | |
| chrX: 66763874- | chrX: 66768086- | TATTATTTCGTGGGCTGTTGAAGAAACAGCTTCTTACCAGGCTTTACATTCCATTAGGTTTTTAATGTTTGACTTACAAGATTTTCAGAG | 148 |
| 66944119 | 66768205 | GGTTCATTTGATATTGTCAAAGTCTTTTCC | |
| chrX: 66763874- | chrX: 66769706- | CATTTTATTAATTCCTCTTTCCTTCTCCTCTCTCCTCTCAGTTATCAAGCATTTTTATGACTATCTTACAAGCAACAGTTTGTCTTGTAA | 149 |
| 66944119 | 66769825 | AGCAGAATTTTCCTTTGAAACCAAGACAGA | |
| chrX: 66763874- | chrX: 66766946- | CTTCCTTTCCTCCTGTCCTCAAAGTCTGAGAACCTCAACAGGAATTTGGGCAATTTCTCCTCTTCAGGTCTGTTAGGATTTCACTTTCAG | 150 |
| 66944119 | 66767065 | CCTGCGCAGATTAGAGTCAAAAAGACCGGC | |
| chrX: 66763874- | chrX: 66770186- | ATTTTCTTTGTTAAGTGTTTTCATTCCCATAGTAATTTTTCATCTAGTGTACATATATGCATTTAAAACAAAAATTCTTTGGTCTCCTTA | 151 |
| 66944119 | 66770305 | TGCGTATATGCACTGCGGCTTGTACACGTA | |
| chrX: 66763874- | chrX: 66767846- | ATTGGGGAATTAACTCCTCAGGTAGGCCAGGTGCTGATGTCCCTGTGGACTTTTGTCTTATTCTTTGTTTCTATGGCTGTTTTCTTTTAC | 152 |
| 66944119 | 66767965 | CTGTGACTTCTCCGAAATTTCTTTGTTAGC | |
| chrX: 66763874- | chrX: 66767366- | TTTATTTTCTCTGTGGATTATAACAGATCCACAGCCCCCTACTTCAGGTTTGCATCAGATCTATAAAGAGGAGAATATTCTTTTAATGTA | 153 |
| 66944119 | 66767485 | CAATTTAATTAGGCTTGACTCTGACTTACA | |
| chrX: 66763874- | chrX: 66770486- | AACTCTGAAAAATAAAAAAAAAAAAAACCAAACAAAAAAATCAAGTTTTGTGAGCTTGGTCAGAGAAGGAAAAGGAAATCTCTCCCTACC | 154 |
| 66944119 | 66770605 | CCCCACCTCCACCATTTTCTCTTTGTCTGC | |
| chrX: 66763874- | chrX: 66767606- | TGCATTTTAGAATATGTGCAGTTAGCTCTAACAAATTGAGTAAGAACTCTTAATGACCTATGAGCCGTAATCTTACCCCAAAGTTTTAAT | 155 |
| 66944119 | 66767725 | TAGCATATGAGAAAAGTGGCAGGCAATTGC | |
| chrX: 66763874- | chrX: 66767726- | ATCGTGCTTATTAAAAATTATTCCTCACCGCAGTTGTTGAGCTTCTTGGAGACCATGCTGAAGATTTTCTCCCCCAGCAAATTAAGATAT | 156 |
| 66944119 | 66767845 | TAGTTTATCTGCTGAGGGAGGACAGACTGA | |
| chrX: 66763874- | chrX: 66769046- | TTCTTAAGCCAAAGCAAGAGTGTCTTCCCTCTCTGTGTTGCATGCATTATGTGCCATGTTTGAGCTAAAAATCTCAAAATTGGGCAGGCT | 157 |
| 66944119 | 66769165 | TCCAATGACCTGTTGGGTCCCTCCCTTTAC | |
| chrX: 66763874- | chrX: 66767066- | CCAATAGCTTCTCAGCGGGTATCCTCCAGAGAGGTAAAGTGAAATTCTCGGTTAGGGAAAGAAAGTGGTCTCTGGGTGCTGAGGTCTGCT | 158 |
| 66944119 | 66767185 | GTGTGAAAGGGTGAACTTCTTTCTCCTGAA | |
| chrX: 66763874- | chrX: 66767786- | AAGATTTTCTCCCCCAGCAAATTAAGATATTAGTTTATCTGCTGAGGGAGGACAGACTGAATTGGGGAATTAACTCCTCAGGTAGGCCAG | 159 |
| 66944119 | 66767905 | GTGCTGATGTCCCTGTGGACTTTTGTCTTA | |
| chrX: 66763874- | chrX: 66767006- | TCTTCAGGTCTGTTAGGATTTCACTTTCAGCCTGCGCAGATTAGAGTCAAAAAGACCGGCCCAATAGCTTCTCAGCGGGTATCCTCCAGA | 160 |
| 66944119 | 66767125 | GAGGTAAAGTGAAATTCTCGGTTAGGGAAA | |
| chrX: 66763874- | chrX: 66769166- | CATTCATGTGTGTGTTTATGTACATAATTTTGTGGAGGGGTTTTTTTAAACCTTAGTAACATCTGCACTCACTCTGTGTTCTTATACATT | 161 |
| 66944119 | 66769285 | TACAGTGTTTCTGCTGAGAGGAGGGAAGAT | |
| chrX: 66763874- | chrX: 66770606- | AGCTTCCTCAAGTGCTGCCTGTCCCCGATTTTCTTTTATTCCACTCCTTTCATGTTTTTGACATTGAAATACAGACTCTTCTTTCCACTT | 162 |
| 66944119 | 66770725 | CTCAGGGTATTTTTCTTATTACACCTGTGG | |
| chrX: 66763874- | chrX: 66768686- | AACTAATGAGAATTTAGCAGTTCCTGCAGAAAGTACAAGTTTATTTTTTTTTTCTGGTTTGTGATTGCTGCACTGAATATGAGGAGTCTA | 163 |
| 66944119 | 66768805 | GTTAAAGGGACAACTGGTGTTCCTGTCTTG | |
| chrX: 66763874- | chrX: 66768806- | TGAGTTGACGAAGACTTTCCATTTCTAGGATATAGAAAATCCTTAAGCCGGTTTATTGAAAATTAATCAATTTAATCAGAATGCAATCAA | 164 |
| 66944119 | 66768925 | TTCCAATACAAAAGTTAGTATTTTCTTTCT | |
| chrX: 66763874- | chrX: 66769586- | AGAACATTTTAATTTTTCTCTTGGTAATTTGTTCTGGTCTCCATAGTAGGTAGTATTTTAGTAGTGCTTTGATATTGACAAGTCTTGCTC | 165 |
| 66944119 | 66769705 | CCTTTCTCTATTAGATTTTTCAAAATAAGG | |
| chrX: 66763874- | chrX: 66769226- | ATCTGCACTCACTCTGTGTTCTTATACATTTACAGTGTTTCTGCTGAGAGGAGGGAAGATGCAAAGGTGGTCTCTTTTACTTAATTTAGC | 166 |
| 66944119 | 66769345 | ATGTGGTTTGAACAGAAGGAAAAATAAAAA | |
| chrX: 66763874- | chrX: 66766706- | CCCCTGGGAGAGCTCAGCAGGGTAAACCTAGAGCTCTCCCGTGGACTCCCGGCCTGCCAGAGGTTTAACCTGAGCTCTCCTAATTTCTGC | 167 |
| 66944119 | 66766825 | TGCGTGCCCTGGGTGCTGATTCCTGCCCTC | |
| chrX: 66763874- | chrX: 66771491- | GCTAGGTAACAAAGTAGAAAGTTAGATTTTCTATGATATTTGTTTACCACGTAGGGGAACCTCTCTAGAGCAATACTCCCAAGCTTTTTC | 168 |
| 66944119 | 66771610 | TTCTTGAAATTTCCCACCTGACAGATAATA | |
| chrX: 66763874- | chrX: 66771251- | GATGTTAAATTCAGAGAAAGTTAACCTTATCTTAAACACAAAGTTGACTTTTAAACAAAATTGCTTATAAAGTTCTGTACAGTTACCAGC | 169 |
| 66944119 | 66771370 | ATTGGTTGCCCTTTGTCGTACGGAAGAGAA | |
| chrX: 66763874- | chrX: 66771191- | AGATATTAAAGAAATTATTCTATCTCCGACTTCCCCTATCAGCATTCCATCAAGTTCTGGGATGTTAAATTCAGAGAAAGTTAACCTTAT | 170 |
| 66944119 | 66771310 | CTTAAACACAAAGTTGACTTTTAAACAAAA | |
| chrX: 66763874- | chrX: 66771071- | AGACCTCAGTAGCTGGATCACAAGCAGTACCCAATATGCATATGAGGGTGCGTGCTGCAAGTGTCCGGCTGGGCTAATCTGCTTAAGCTT | 171 |
| 66944119 | 66771190 | CATAAAAATTAATCATTTGAAAACAAAGAA | |
| chrX: 66763874- | chrX: 66771131- | GTGTCCGGCTGGGCTAATCTGCTTAAGCTTCATAAAAATTAATCATTTGAAAACAAAGAAAGATATTAAAGAAATTATTCTATCTCCGAC | 172 |
| 66944119 | 66771250 | TTCCCCTATCAGCATTCCATCAAGTTCTGG | |
| chrX: 66763874- | chrX: 66771731- | CTGCCAACCTGCAGGCCACACCAGGATTGAGGGCAGCTCATCTCGATAAATTTATAGCATTAAAGTGCTGGGTCATTTGAGAATGTTGTC | 173 |
| 66944119 | 66771850 | AATTTAGGTTACTTAGTACCTAAGTTTTAT | |
| chrX: 66763874- | chrX: 66771671- | TGAATTATTGCTTGCACACTCATGGGTGATGCTACTCCCTCTCTCTCATGGCAATTCTTGCTGCCAACCTGCAGGCCACACCAGGATTGA | 174 |
| 66944119 | 66771790 | GGGCAGCTCATCTCGATAAATTTATAGCAT | |
| chrX: 66763874- | chrX: 66771371- | TTATGAAATCTCATATTTACATAGCATTCTTCCAAAAAAAGAGACGGTGTTTTCCAGTTTATTCACTGCATTCGTGTAAGTGTGAGTAGG | 175 |
| 66944119 | 66771490 | CCAGGAGGGGTGCTTAGTGATTACCCTTTT | |
| chrX: 66763874- | chrX: 66771551- | CTCTCTAGAGCAATACTCCCAAGCTTTTTCTTCTTGAAATTTCCCACCTGACAGATAATACTTTAGATTGTTGCTCTTAAGGACTTCTCT | 176 |
| 66944119 | 66771670 | CAGTAGCTGCTACATAGAGATGATTGTCCG | |
| chrX: 66763874- | chrX: 66771431- | ATTCACTGCATTCGTGTAAGTGTGAGTAGGCCAGGAGGGGTGCTTAGTGATTACCCTTTTGCTAGGTAACAAAGTAGAAAGTTAGATTTT | 177 |
| 66944119 | 66771550 | CTATGATATTTGTTTACCACGTAGGGGAAC | |
| chrX: 66763874- | chrX: 66771011- | GCTACTTTCTCTCAGAGCCTGAGAGACAGCTCTGAGACACTTCCCAGGTCTGTTCGGTTCAGACCTCAGTAGCTGGATCACAAGCAGTAC | 178 |
| 66944119 | 66771130 | CCAATATGCATATGAGGGTGCGTGCTGCAA | |
| chrX: 66763874- | chrX: 66771311- | TTGCTTATAAAGTTCTGTACAGTTACCAGCATTGGTTGCCCTTTGTCGTACGGAAGAGAATTATGAAATCTCATATTTACATAGCATTCT | 179 |
| 66944119 | 66771430 | TCCAAAAAAAGAGACGGTGTTTTCCAGTTT | |
| chrX: 66763874- | chrX: 66771611- | CTTTAGATTGTTGCTCTTAAGGACTTCTCTCAGTAGCTGCTACATAGAGATGATTGTCCGTGAATTATTGCTTGCACACTCATGGGTGAT | 180 |
| 66944119 | 66771730 | GCTACTCCCTCTCTCTCATGGCAATTCTTG | |
| chrX: 66763874- | chrX: 66773884- | TATAAATTCGAACTGGTAATCCCATCCCCTTTCGGGATGAATAGGAGAGTGTTTTTAAATGTTCATCTCTTTAGAGAACAGCAGGAAAGA | 181 |
| 66944119 | 66774003 | AGCCTAGTAAGGTTTGGGTAGTTTATAATC | |
| chrX: 66763874- | chrX: 66774364- | GCTAATCAGCTACAACTGTAATGTCCTGATAATTGTGAATTAACTGCAGGGCACCCAGCAAAAGGTTTAGTTATAATCTAATAGCTGTCT | 182 |
| 66944119 | 66774483 | GTAGAGATTAGCCTAATAAAGGGATTTTTT | |
| chrX: 66763874- | chrX: 66774004- | CCTTTTTTAGAATTTGGATTTGGGAACTATTAGCAAGGCAGTGAGTAATAATAATAATTTCTATATAGAAAACTAACATGTAGAGGTGAC | 183 |
| 66944119 | 66774123 | AAATGAAATCACTAGCTATATTAGGCTTAT | |
| chrX: 66763874- | chrX: 66773824- | TTACTTCATTTCATCCTGTATGAAGGCTGCATGGGGACATTCTTCTCAGTTTTACTCAGCTATAAATTCGAACTGGTAATCCCATCCCCT | 184 |
| 66944119 | 66773943 | TTCGGGATGAATAGGAGAGTGTTTTTAAAT | |
| chrX: 66763874- | chrX: 66774304- | AAATTCATCTATACACAGACTGAACTTTGTCTTCATTAACACTCTAGGCTAAGGGTCATAGCTAATCAGCTACAACTGTAATGTCCTGAT | 185 |
| 66944119 | 66774423 | AATTGTGAATTAACTGCAGGGCACCCAGCA | |
| chrX: 66763874- | chrX: 66773944- | GTTCATCTCTTTAGAGAACAGCAGGAAAGAAGCCTAGTAAGGTTTGGGTAGTTTATAATCCCTTTTTTAGAATTTGGATTTGGGAACTAT | 186 |
| 66944119 | 66774063 | TAGCAAGGCAGTGAGTAATAATAATAATTT | |
| chrX: 66763874- | chrX: 66774124- | GTTTAGGTTATCGTAAGCAGCTAAAATCATAATTTTATGTTTTTATATGTTGTCCTTTGGACAAAGTAAATTCCAGTACTCCTTCTGATG | 187 |
| 66944119 | 66774243 | TGCATTTCTAGATGGGGAAAGGATTCATTT | |
| chrX: 66763874- | chrX: 66774064- | CTATATAGAAAACTAACATGTAGAGGTGACAAATGAAATCACTAGCTATATTAGGCTTATGTTTAGGTTATCGTAAGCAGCTAAAATCAT | 188 |
| 66944119 | 66774183 | AATTTTATGTTTTTATATGTTGTCCTTTGG | |
| chrX: 66763874- | chrX: 66774244- | ACTCTCATATAATTTAAGCTTCTTTTTAGGGATGTACTCCATAGCCATGAAGCAAAGATAAAATTCATCTATACACAGACTGAACTTTGT | 189 |
| 66944119 | 66774363 | CTTCATTAACACTCTAGGCTAAGGGTCATA | |
| chrX: 66763874- | chrX: 66773764- | TTGAATGCTTCTGGGACTTATGGGGAAGAGGGCTTCTGCTGCTGCACTGAAAGTTAAAGCTTACTTCATTTCATCCTGTATGAAGGCTGC | 190 |
| 66944119 | 66773883 | ATGGGGACATTCTTCTCAGTTTTACTCAGC | |
| chrX: 66763874- | chrX: 66774184- | ACAAAGTAAATTCCAGTACTCCTTCTGATGTGCATTTCTAGATGGGGAAAGGATTCATTTACTCTCATATAATTTAAGCTTCTTTTTAGG | 191 |
| 66944119 | 66774303 | GATGTACTCCATAGCCATGAAGCAAAGATA | |
| chrX: 66763874- | chrX: 66775352- | CAATAATATGAGTAATAAATTAAAATTGAAGGTGATTAATGGCTCTGAATTTGACATAAGAGTTGTTTTCCTGCCTTCTAAGTTTCCATT | 192 |
| 66944119 | 66775471 | GATCCTGATGAATTGCACAAACCAAACAAT | |
| chrX: 66763874- | chrX: 66775532- | CATTCTGAAATGGCCTACCACCTAACATGGGCTCTGTTCTCTGCAGGTTGAGTAGGTTCCTTGCTTGTGGAACTGTAGTCCCGCTATTTG | 193 |
| 66944119 | 66775651 | GCCGCTAGGGGGACTGCAAGTGCCCCGTGG | |
| chrX: 66763874- | chrX: 66774992- | ATCCTGGATTGTACGGTATGGATTTCTTAAAATGTAGATATTTTAAAAAAAAAGAGGAATGAATCAATAGAGGCTGAAGTGGTCAGCAAT | 194 |
| 66944119 | 66775111 | GTTACCTGTGGCTGCTTTTAATCCTTCGTG | |
| chrX: 66763874- | chrX: 66775292- | TGGCTATATCATTGCACTTGGTGGGTACATGTTTATGATGTCTTTATCTGAACAAGTCAGCAATAATATGAGTAATAAATTAAAATTGAA | 195 |
| 66944119 | 66775411 | GGTGATTAATGGCTCTGAATTTGACATAAG | |
| chrX: 66763874- | chrX: 66775712- | ATCGCAGGATAACTTGAACCAGAACTGCCTAGCACCAGACAATAAATAAGCTACTATGGTACTTACTGTTTCATTTGGGATGTTGTTTCT | 196 |
| 66944119 | 66775831 | CGAAGTGGCAAGCATTTTTTAGTAATATTT | |
| chrX: 66763874- | chrX: 66775172- | AAATAACAGAAGTTAGTACCACTGGAAAGAATGAACTGGAGGAATGGGTTGAAATCTATTTCTGCTTATTCAATAGTGCACCCCAGTCAA | 197 |
| 66944119 | 66775291 | GTTAGTTGCCAATTTCTTCTTCAGTTTCTT | |
| chrX: 66763874- | chrX: 66775412- | AGTTGTTTTCCTGCCTTCTAAGTTTCCATTGATCCTGATGAATTGCACAAACCAAACAATTCGGGGAGTAAGGGGGCACATGATGATCTT | 198 |
| 66944119 | 66775531 | ATAAGAGCTTTGCTGTATTAGACAACGTAA | |
| chrX: 66763874- | chrX: 66775592- | TTGCTTGTGGAACTGTAGTCCCGCTATTTGGCCGCTAGGGGGACTGCAAGTGCCCCGTGGCAGGATTTCCCTGGGAATGGTGAGCCTCCA | 199 |
| 66944119 | 66775711 | TTGATGGTTTCAACACACAGCCAAGGCCCT | |
| chrX: 66763874- | chrX: 66774932- | accaaacaaCCAAAAACCAAACAGAAATGCAGTATCATCATGCCATGATGCCTGTATGAGATCCTGGATTGTACGGTATGGATTTCTTAA | 200 |
| 66944119 | 66775051 | AATGTAGATATTTTAAAAAAAAAGAGGAAT | |
| chrX: 66763874- | chrX: 66775772- | ACTTACTGTTTCATTTGGGATGTTGTTTCTCGAAGTGGCAAGCATTTTTTAGTAATATTTTGACTTTTTAATACCTTTCTTTGCATATGG | 201 |
| 66944119 | 66775891 | AGCAGAAAACAGTGACACTGGAtatattca | |
| chrX: 66763874- | chrX: 66775232- | TCTGCTTATTCAATAGTGCACCCCAGTCAAGTTAGTTGCCAATTTCTTCTTCAGTTTCTTTGGCTATATCATTGCACTTGGTGGGTACAT | 202 |
| 66944119 | 66775351 | GTTTATGATGTCTTTATCTGAACAAGTCAG | |
| chrX: 66763874- | chrX: 66775472- | TCGGGGAGTAAGGGGGCACATGATGATCTTATAAGAGCTTTGCTGTATTAGACAACGTAACATTCTGAAATGGCCTACCACCTAACATGG | 203 |
| 66944119 | 66775591 | GCTCTGTTCTCTGCAGGTTGAGTAGGTTCC | |
| chrX: 66763874- | chrX: 66775652- | CAGGATTTCCCTGGGAATGGTGAGCCTCCATTGATGGTTTCAACACACAGCCAAGGCCCTATCGCAGGATAACTTGAACCAGAACTGCCT | 204 |
| 66944119 | 66775771 | AGCACCAGACAATAAATAAGCTACTATGGT | |
| chrX: 66763874- | chrX: 66775052- | GAATCAATAGAGGCTGAAGTGGTCAGCAATGTTACCTGTGGCTGCTTTTAATCCTTCGTGGAAGTAAGTAGGAGCATGTCTAAACTCAAG | 205 |
| 66944119 | 66775171 | CAATAGATTAAAGATCTTGATGTATATTTT | |
| chrX: 66763874- | chrX: 66775112- | GAAGTAAGTAGGAGCATGTCTAAACTCAAGCAATAGATTAAAGATCTTGATGTATATTTTAAATAACAGAAGTTAGTACCACTGGAAAGA | 206 |
| 66944119 | 66775231 | ATGAACTGGAGGAATGGGTTGAAATCTATT | |
| chrX: 66763874- | chrX: 66776189- | GAGAGGCAAAAGAACCACAAGTGGTATCAATACTAGAAATTTATGAATTTCTTAAGGCTTCTAGGTTTGTTACCCATCCACCAGACTGAT | 207 |
| 66944119 | 66776308 | GGATTTGGTTGTGTGAGAGTTCTGGGTGCC | |
| chrX: 66763874- | chrX: 66777704- | AATCACACTGAAATATTATTATTTTTACTGAACCACATACCAAAATATTTTTCCTGTAAAAACACAGTAAGTGAACTTTTAAAGGCAATT | 208 |
| 66944119 | 66777823 | GAGCTTTTAACAAAGCTAGAATCTACAGAG | |
| chrX: 66763874- | chrX: 66777644- | AGGTAATTAAACTAAATAGTGAATAAAACTGGGAAACTATACAAATTGGTTGCTCTCCCCAATCACACTGAAATATTATTATTTTTACTG | 209 |
| 66944119 | 66777763 | AACCACATACCAAAATATTTTTCCTGTAAA | |
| chrX: 66763874- | chrX: 66777584- | GGGCAAGAGTGAGTTTTTTGTTATTGTTTTTTGTTTGTTTCTTTGCCCAAACCTAAAACCAGGTAATTAAACTAAATAGTGAATAAAACT | 210 |
| 66944119 | 66777703 | GGGAAACTATACAAATTGGTTGCTCTCCCC | |
| chrX: 66763874- | chrX: 66777979- | cCTAGTCAGAGCCTGTTTTAAGGGTTAGTCGTATGTTGTTTTCTTGAAAAAAGTTACATTGGAAAAGTGAAAATTCTTTGGTCCATACTG | 211 |
| 66944119 | 66778098 | AGAACAAAGAATTATACATAATCATATATa | |
| chrX: 66763874- | chrX: 66778537- | AGAGATCCCAAGGAGGTTGATCTCCGACTGCTACAAACCTGGGCAATTCAATGCCTGCTTAAATAGGAGAGTTAAGATAAGAAAAATAAA | 212 |
| 66944119 | 66778656 | ATTGCCAATTTTTACAgicagacattgttt | |
| chrX: 66763874- | chrX: 66778477- | AGTTGATGACTCATGATAGGGATTGGAAAACAGGACTACAGGAATTATTGAAAAGGGCCTAGAGATCCCAAGGAGGTTGATCTCCGACTG | 213 |
| 66944119 | 66778596 | CTACAAACCTGGGCAATTCAATGCCTGCTT | |
| chrX: 66763874- | chrX: 66778417- | TAGGTGGGTGGCCTAGAGGTTAAAGTAGAGGCAGAGTGATGGAAAGGGGTGGTTAGAAGAAGTTGATGACTCATGATAGGGATTGGAAAA | 214 |
| 66944119 | 66778536 | CAGGACTACAGGAATTATTGAAAAGGGCCT | |
| chrX: 66763874- | chrX: 66778357- | GGTGACGTAGGTGACTTCATTATGCTCTGCCCCTATTATAGTCCACTGATCCTCACCAAATAGGTGGGTGGCCTAGAGGTTAAAGTAGAG | 215 |
| 66944119 | 66778476 | GCAGAGTGATGGAAAGGGGTGGTTAGAAGA | |
| chrX: 66763874- | chrX: 66778297- | ctactgtgcaatacCAGGAGCTTCTCAGATTACCCTTCACCTTTACCAACCCAAATGACTGGTGACGTAGGTGACTTCATTATGCTCTGC | 216 |
| 66944119 | 66778416 | CCCTATTATAGTCCACTGATCCTCACCAAA | |
| chrX: 66763874- | chrX: 66778860- | TATGGAATGAGGTAATTTTCTTATAACAGAAAGTTTTTAAAATGCAAAAACATTGTGCCTGAACTTCAAACACTGAACAACTCATATCCT | 217 |
| 66944119 | 66778979 | TAATATGCACCAGTTTCTTTTAAGCACTCT | |
| chrX: 66763874- | chrX: 66779443- | TGTCTCTCCAGGTTTTCTTTTAAAGAAGGTAGTCTTGCTAAATGATAACTATTTCAGCATTTATTTGAAAATGGGCAGTGCAGGAGAGAA | 218 |
| 66944119 | 66779562 | AGAATTTTTCCAAGCTTGTCACATTGGGCC | |
| chrX: 66763874- | chrX: 66779563- | ACCTCTCTGAAGCATTGTCCAACTTCTAATTAGATGAGGAGACTGCATAAACCAAGAGTTGAGAGTAAAGATGGAAACACTTGATGTTTG | 219 |
| 66944119 | 66779682 | GTGTTTGGGTGCAGAAAGGATTCCAGAACA | |
| chrX: 66763874- | chrX: 66780163- | AGGATTAGTGCAGTGATCCCACGTCCTTTCTCTCTAGCTCTCTCTGCTACTCTCTAATTCCTATTGTATTTGTGCCACCAGATCTTTCCA | 220 |
| 66944119 | 66780282 | AAGTTTAGCTCCAATCttgtctgtatactg | |
| chrX: 66763874- | chrX: 66779383- | TGTGGTTGTCAGATAACCTTTTTGTTACTGTGGAAATGGAAGCAGGCTACTGCAAAAATCTGTCTCTCCAGGTTTTCTTTTAAAGAAGGT | 221 |
| 66944119 | 66779502 | AGTCTTGCTAAATGATAACTATTTCAGCAT | |
| chrX: 66763874- | chrX: 66779923- | TCTGGGCTTCAGTGAGGTGCTAGATTGACTCAAAATATGGCAGGTCAGATGTGGGATTGAGCAGGGTGGACTCTTCTCTACCCTTCCCAA | 222 |
| 66944119 | 66780042 | TTCAGAGTTCCCCATCAAAGATGATCTCAT | |
| chrX: 66763874- | chrX: 66779983- | GCAGGGTGGACTCTTCTCTACCCTTCCCAATTCAGAGTTCCCCATCAAAGATGATCTCATAGTGTTTGAAAAACCAAGCTGAAGGCTTTG | 223 |
| 66944119 | 66780102 | GGAATTAGGGTGCTGAAGGGATATGCTGTT | |
| chrX: 66763874- | chrX: 66779863- | GACCAAAAATTTTAACCATGCTCATACTTTCATATGGTATGTAGTTTGCTTTAGACATTTTCTGGGCTTCAGTGAGGTGCTAGATTGACT | 224 |
| 66944119 | 66779982 | CAAAATATGGCAGGTCAGATGTGGGATTGA | |
| chrX: 66763874- | chrX: 66780103- | TCCCAAAGCCTTCTCAGTCATTCCTTCTCCCCCCAGTTCAGATTCTTAACACCTCTTTCCAGGATTAGTGCAGTGATCCCACGTCCTTTC | 225 |
| 66944119 | 66780222 | TCTCTAGCTCTCTCTGCTACTCTCTAATTC | |
| chrX: 66763874- | chrX: 66779143- | TTCACCTTTTAATTACCTGTCTCCATCAACAAGATTGGACAGAGAATTGGGAGAGTGAGCAGAGTCCATTTCCTTCCAGAGACTGGACAA | 226 |
| 66944119 | 66779262 | AAGGAACAAAATGTTAGGAAAAAATGTCAG | |
| chrX: 66763874- | chrX: 66779503- | TTATTTGAAAATGGGCAGTGCAGGAGAGAAAGAATTTTTCCAAGCTTGTCACATTGGGCCACCTCTCTGAAGCATTGTCCAACTTCTAAT | 227 |
| 66944119 | 66779622 | TAGATGAGGAGACTGCATAAACCAAGAGTT | |
| chrX: 66763874- | chrX: 66779263- | CATGTGGGATTTGTGGGATTTACACTAAATAAGAAGGGACACTTCCCAGGACTGACAAGATGCTACCTCCGTCCCTCTAGGCCCCAATGT | 228 |
| 66944119 | 66779382 | GTTGTGCAGGATCCCATAGGAAGTCATGAA | |
| chrX: 66763874- | chrX: 66779323- | TGCTACCTCCGTCCCTCTAGGCCCCAATGTGTTGTGCAGGATCCCATAGGAAGTCATGAATGTGGTTGTCAGATAACCTTTTTGTTACTG | 229 |
| 66944119 | 66779442 | TGGAAATGGAAGCAGGCTACTGCAAAAATC | |
| chrX: 66763874- | chrX: 66779803- | TTTTGAATAAAAAAGACCAAATTAGACTGAGATATTTCAGTCACCAACTATCTAATAATAGACCAAAAATTTTAACCATGCTCATACTTT | 230 |
| 66944119 | 66779922 | CATATGGTATGTAGTTTGCTTTAGACATTT | |
| chrX: 66763874- | chrX: 66779623- | GAGAGTAAAGATGGAAACACTTGATGTTTGGTGTTTGGGTGCAGAAAGGATTCCAGAACATGTTTTGGGTCTCTTTACTCTGTCCATCCC | 231 |
| 66944119 | 66779742 | TCCTTCCCTTTCATCTTTGTTTAAAAACCA | |
| chrX: 66763874- | chrX: 66779083- | ttgggctcccttctAACATTGACTTGTCTCCCTCCATTCCTCCTCCGTATTGTTCTGCCCTTCACCTTTTAATTACCTGTCTCCATCAAC | 232 |
| 66944119 | 66779202 | AAGATTGGACAGAGAATTGGGAGAGTGAGC | |
| chrX: 66763874- | chrX: 66779743- | CAGTTAGCAAATGTGTAGTCTGTTTGCAATTGTTCATCTGAAAAATTTGTTTGATCAGCCTTTTGAATAAAAAAGACCAAATTAGACTGA | 233 |
| 66944119 | 66779862 | GATATTTCAGTCACCAACTATCTAATAATA | |
| chrX: 66763874- | chrX: 66779203- | AGAGTCCATTTCCTTCCAGAGACTGGACAAAAGGAACAAAATGTTAGGAAAAAATGTCAGCATGTGGGATTTGTGGGATTTACACTAAAT | 234 |
| 66944119 | 66779322 | AAGAAGGGACACTTCCCAGGACTGACAAGA | |
| chrX: 66763874- | chrX: 66779683- | TGTTTTGGGTCTCTTTACTCTGTCCATCCCTCCTTCCCTTTCATCTTTGTTTAAAAACCACAGTTAGCAAATGTGTAGTCTGTTTGCAAT | 235 |
| 66944119 | 66779802 | TGTTCATCTGAAAAATTTGTTTGATCAGCC | |
| chrX: 66763874- | chrX: 66780043- | AGTGTTTGAAAAACCAAGCTGAAGGCTTTGGGAATTAGGGTGCTGAAGGGATATGCTGTTTCCCAAAGCCTTCTCAGTCATTCCTTCTCC | 236 |
| 66944119 | 66780162 | CCCCAGTTCAGATTCTTAACACCTCTTTCC | |
| chrX: 66763874- | chrX: 66780514- | CTTACAAAACTCTTAAAACCCAGCCAAAAGGATCTAGTCACTGTCACTTTAAACCATCCTCACTCTCTTGTTTTTTGAACATGTTATTTT | 237 |
| 66944119 | 66780633 | TCTTATAATCCCTTTGACCTTGAAGGCTAT | |
| chrX: 66763874- | chrX: 66780634- | CCCAATTTCAATACTATCCATTCTTCTATGACAGCCCCCTACAAAATGAATATTCTCAACCTCCCAACCCAAGGAGAAGTGATCTATATG | 238 |
| 66944119 | 66780753 | ACACAATATGGTTGAAAGAATGTTGgcttc | |
| chrX: 66763874- | chrX: 66780454- | TCTGGTGTTCAAGTCCTTTTATGATCTGCTTATTTTTCCAGCCTGAATTCCTGGAGTTCCCTTACAAAACTCTTAAAACCCAGCCAAAAG | 239 |
| 66944119 | 66780573 | GATCTAGTCACTGTCACTTTAAACCATCCT | |
| chrX: 66763874- | chrX: 66780574- | CACTCTCTTGTTTTTTGAACATGTTATTTTTCTTATAATCCCTTTGACCTTGAAGGCTATCCCAATTTCAATACTATCCATTCTTCTATG | 240 |
| 66944119 | 66780693 | ACAGCCCCCTACAAAATGAATATTCTCAAC | |
| chrX: 66763874- | chrX: 66780394- | catgaTTACAATTTCAGCTTCTCCCCATTGGCTTATGGGTTAAAGTCCAAATTATTTAAATCTGGTGTTCAAGTCCTTTTATGATCTGCT | 241 |
| 66944119 | 66780513 | TATTTTTCCAGCCTGAATTCCTGGAGTTCC | |
| chrX: 66763874- | chrX: 66780879- | CTTTTTTCTTTCTCCATCAATATGGTACTTAGTCCCTTAAATCAGAAGTACTTGTGTTAATGTCTGATAACGTCCTTCTAAATATACCTC | 242 |
| 66944119 | 66780998 | TAAACATCTGTCTCTCTTTAGGGCAAAGGT | |
| chrX: 66763874- | chrX: 66782473- | tgacatcccCATCAGATCAGAGACCTAAGCAAATCTTGGGTCCCTTGCTTACTCCAAGGGCTTTCACTCCTCGTATAGGAGGAGCTAAAG | 243 |
| 66944119 | 66782592 | AAATGTACAAGCAGCACCACAAtaggatca | |
| chrX: 66763874- | chrX: 66782955- | AAAGCCTATAGATGACTCCAAAATGCCCATTTGGATGATATGAGGCATACTTTGTGTAGTTAAGGATTTTAAATACATAACAGAGAGGCT | 244 |
| 66944119 | 66783074 | GAAGGGCCTTCGGGAAAGAAGCTGGGGTAA | |
| chrX: 66763874- | chrX: 66783581- | GTTGATGTGTGCTTCAAAGTGTGAGACAGAGCTAGTCTGAGGAGAGAGGGAGAGTGAGAAGATTCCTCTTCTTGGCCAGAGGTCATGGTC | 245 |
| 66944119 | 66783700 | TTCCACAAGGAACAGAATGACTCAATGCAA | |
| chrX: 66763874- | chrX: 66783701- | ATTATGGGACCTCTTTGAGTTTGGGGCCCCTACATTTAAACTAGTAACTCCGTTGCACATATTGGCACCCTTCCCCCAACAAAATTACTG | 246 |
| 66944119 | 66783820 | GGCAGGAATTTTCTTGAATCCTTCCGTGGC | |
| chrX: 66763874- | chrX: 66783521- | TTGCTTCGGTAATGCAAGTTATTAAGTTACTTCCCTCAGCCCAGCTGAAATCTCTTATTGGTTGATGTGTGCTTCAAAGTGTGAGACAGA | 247 |
| 66944119 | 66783640 | GCTAGTCTGAGGAGAGAGGGAGAGTGAGAA | |
| chrX: 66763874- | chrX: 66783761- | ATTGGCACCCTTCCCCCAACAAAATTACTGGGCAGGAATTTTCTTGAATCCTTCCGTGGCCTGGAATGATCTCCCTTCTCATCCTTGTGA | 248 |
| 66944119 | 66783880 | TCCACACAGCTGGCAAATGGCAGGCAGCAG | |
| chrX: 66763874- | chrX: 66783401- | ACATGGGCTTTTCCCCAGGCCACTGCTGCCTGGCTTCCCCTTCCACAAAGCTTTGAGTCTCCAAAATGCTTTGGCTGGAATGTAAGCGTG | 249 |
| 66944119 | 66783520 | AGGTCATTGCAGATAACAGGGGAGCATGAT | |
| chrX: 66763874- | chrX: 66783641- | GATTCCTCTTCTTGGCCAGAGGTCATGGTCTTCCACAAGGAACAGAATGACTCAATGCAAATTATGGGACCTCTTTGAGTTTGGGGCCCC | 250 |
| 66944119 | 66783760 | TACATTTAAACTAGTAACTCCGTTGCACAT | |
| chrX: 66763874- | chrX: 66783881- | AACAAAAACAAGCCTCTTAGCATATAGGGAGAGAAAGAGTCACAGCAGTACTGAATTTGCTTGGGAACCTAATGTTAACAAAGGACCTTC | 251 |
| 66944119 | 66784000 | CTCTCAACACCCCAAACAGATTAAAACATT | |
| chrX: 66763874- | chrX: 66783941- | TTGGGAACCTAATGTTAACAAAGGACCTTCCTCTCAACACCCCAAACAGATTAAAACATTTTTTTAACAGCAAGTTGTGTCTCGGAGCAG | 252 |
| 66944119 | 66784060 | CTCTTTGCTTGGGTATATTTAAAGATCTGC | |
| chrX: 66763874- | chrX: 66784001- | TTTTTAACAGCAAGTTGTGTCTCGGAGCAGCTCTTTGCTTGGGTATATTTAAAGATCTGCTGAGTCATTTAAGAGCAGGCTGGCATATCC | 253 |
| 66944119 | 66784120 | TAAGAGGCAAGGACTATACCCCAGTCTATG | |
| chrX: 66763874- | chrX: 66784181- | TCCACTTCCATCTCCCACGACTCTGGAGAGCATCTACTAAGCCTTCTTATTCTATCAACTTTGAACTCCTCAGTGTATAATAGAGTAAGG | 254 |
| 66944119 | 66784300 | GTGAGAGGGAAGGAGCAGTCGTACCAGTGt | |
| chrX: 66763874- | chrX: 66784121- | GGGGAGTAAGTTGAGAGGTGAAATCTGTTTGGCTTTCTCCCATGGAAACAAACAAGGTGATCCACTTCCATCTCCCACGACTCTGGAGAG | 255 |
| 66944119 | 66784240 | CATCTACTAAGCCTTCTTATTCTATCAACT | |
| chrX: 66763874- | chrX: 66783341- | tAGTAGTAGTAGTAGTCCAGACAAACAGAGCTTGGGAAAACGCTAGACTCTGGCTGACATACATGGGCTTTTCCCCAGGCCACTGCTGCC | 256 |
| 66944119 | 66783460 | TGGCTTCCCCTTCCACAAAGCTTTGAGTCT | |
| chrX: 66763874- | chrX: 66783461- | CCAAAATGCTTTGGCTGGAATGTAAGCGTGAGGTCATTGCAGATAACAGGGGAGCATGATTTGCTTCGGTAATGCAAGTTATTAAGTTAC | 257 |
| 66944119 | 66783580 | TTCCCTCAGCCCAGCTGAAATCTCTTATTG | |
| chrX: 66763874- | chrX: 66784061- | TGAGTCATTTAAGAGCAGGCTGGCATATCCTAAGAGGCAAGGACTATACCCCAGTCTATGGGGGAGTAAGTTGAGAGGTGAAATCTGTTT | 258 |
| 66944119 | 66784180 | GGCTTTCTCCCATGGAAACAAACAAGGTGA | |
| chrX: 66763874- | chrX: 66783821- | CTGGAATGATCTCCCTTCTCATCCTTGTGATCCACACAGCTGGCAAATGGCAGGCAGCAGAACAAAAACAAGCCTCTTAGCATATAGGGA | 259 |
| 66944119 | 66783940 | GAGAAAGAGTCACAGCAGTACTGAATTTGC | |
| chrX: 66763874- | chrX: 66785205- | CCCTCTTGCATTTTGTTTATCGATGGTTTTCAAAGGACTTAGAGGCTGGCTTTGTTATAGTTAGTTGGTAAGAGAAATGGTGGAGGACCG | 260 |
| 66944119 | 66785324 | GAAAATGGGAGTGGAACGAATGAGCATGTT | |
| chrX: 66763874- | chrX: 66784965- | GCCATCTCCTTGCCATCAAAACCATCCTCACAGACCCTTCTGAAACCACTTCTAGGAAGGGAAATCACAATGGATCCATGAAGGATGCTT | 261 |
| 66944119 | 66785084 | TCTGGATGACTTTAAAAGATTGGTATTAAG | |
| chrX: 66763874- | chrX: 66785025- | GAAATCACAATGGATCCATGAAGGATGCTTTCTGGATGACTTTAAAAGATTGGTATTAAGATATTTTATCAGTGGTAGCAACACTGACTT | 262 |
| 66944119 | 66785144 | ATTCAGGCAGCCATGCCCCGGATCTATAAG | |
| chrX: 66763874- | chrX: 66785085- | ATATTTTATCAGTGGTAGCAACACTGACTTATTCAGGCAGCCATGCCCCGGATCTATAAGAAATCAGGTAAGCTAAAAGTTGCTTGAGCT | 263 |
| 66944119 | 66785204 | GGCAGGAGACCTAGTTCTCTTTTTTCCTTT | |
| chrX: 66763874- | chrX: 66784725- | CCAATTTTCCCCCTGGTTTTCATCTTGTAAATCTGTAATCCTAAAAATTAGCAAAACCTAGAGCTTCTCTTTGGTCCTGGCCTGTTTGAA | 264 |
| 66944119 | 66784844 | CCCTGTTCCACAGACCCCAATCTTCTTTCT | |
| chrX: 66763874- | chrX: 66784665- | GTGGAGAAATTAGCAGTTACCAACTGTTCATTATAGGTACACATTGGGGTTTCCTTAGAGCCAATTTTCCCCCTGGTTTTCATCTTGTAA | 265 |
| 66944119 | 66784784 | ATCTGTAATCCTAAAAATTAGCAAAACCTA | |
| chrX: 66763874- | chrX: 66784605- | GCTAGAAAGTGAAATAATTTTTTTTCTACTTTATTCTCCACTGCACTTCTAAATTTATTAGTGGAGAAATTAGCAGTTACCAACTGTTCA | 266 |
| 66944119 | 66784724 | TTATAGGTACACATTGGGGTTTCCTTAGAG | |
| chrX: 66763874- | chrX: 66784905- | TCTCTAGCACCTCAGACTGTCTTCCCACAGTGGCACAGCCTCCCACTCCACTTTCACTGTGCCATCTCCTTGCCATCAAAACCATCCTCA | 267 |
| 66944119 | 66785024 | CAGACCCTTCTGAAACCACTTCTAGGAAGG | |
| chrX: 66763874- | chrX: 66784845- | TGTTTGAGGCAACTATCCTTCTCTTTGCCCACCGCCATTTTCCTTCATCTACTTTTCCCTTCTCTAGCACCTCAGACTGTCTTCCCACAG | 268 |
| 66944119 | 66784964 | TGGCACAGCCTCCCACTCCACTTTCACTGT | |
| chrX: 66763874- | chrX: 66784785- | GAGCTTCTCTTTGGTCCTGGCCTGTTTGAACCCTGTTCCACAGACCCCAATCTTCTTTCTTGTTTGAGGCAACTATCCTTCTCTTTGCCC | 269 |
| 66944119 | 66784904 | ACCGCCATTTTCCTTCATCTACTTTTCCCT | |
| chrX: 66763874- | chrX: 66785145- | AAATCAGGTAAGCTAAAAGTTGCTTGAGCTGGCAGGAGACCTAGTTCTCTTTTTTCCTTTCCCTCTTGCATTTTGTTTATCGATGGTTTT | 270 |
| 66944119 | 66785264 | CAAAGGACTTAGAGGCTGGCTTTGTTATAG | |
| chrX: 66763874- | chrX: 66784485- | TTGTAATGGCCCTGCTATTGGTAGCTGAGAGTAGCATGGAAGTGTCAGGTTGATGGGTTCATTTAATTCTTTTCTTTTCAGTTTCAGTCA | 271 |
| 66944119 | 66784604 | CATGCATTGTTACCATGGCATATGACAGTT | |
| chrX: 66763874- | chrX: 66784545- | ATTTAATTCTTTTCTTTTCAGTTTCAGTCACATGCATTGTTACCATGGCATATGACAGTTGCTAGAAAGTGAAATAATTTTTTTTCTACT | 272 |
| 66944119 | 66784664 | TTATTCTCCACTGCACTTCTAAATTTATTA | |
| chrX: 66763874- | chrX: 66785821- | cactgctcccagccCCAGACCATTAATTTTTGACAGTAAGTCCAACTTTTTTCAAGTTCACAGCTCAGATTTGCTATTGAATGAATGAGT | 273 |
| 66944119 | 66785940 | ATATATGTCATTTGGGAACATTCTTTCCAA | |
| chrX: 66763874- | chrX: 66785941- | CTTTTGGTTGAAGATTTGTTTTATCACTTGTGAAAATTTTTTTTCATTCTTAGCAATGTCAGTTTAGTTAAATGAGCATTTCATTTGCGA | 274 |
| 66944119 | 66786060 | ATTCACTAATTAATTATtttattcatcaat | |
| chrX: 66763874- | chrX: 66785881- | CAGCTCAGATTTGCTATTGAATGAATGAGTATATATGTCATTTGGGAACATTCTTTCCAACTTTTGGTTGAAGATTTGTTTTATCACTTG | 275 |
| 66944119 | 66786000 | TGAAAATTTTTTTTCATTCTTAGCAATGTC | |
| chrX: 66763874- | chrX: 66786358- | TTCAGCCTCTGTGTGTGTATTTGTGTGTGTGAAGGTTGAGTGTGTGATGATGGATGGGGCTGCGAGATTGTTAAGTAGGATCTATGGGGG | 276 |
| 66944119 | 66786477 | GCCTTAAATGGTCCTGGTGAGTCCCAACTT | |
| chrX: 66763874- | chrX: 66786418- | TGCGAGATTGTTAAGTAGGATCTATGGGGGGCCTTAAATGGTCCTGGTGAGTCCCAACTTTCTGGTTATGTATTTGAGTAGAGTATGGGG | 277 |
| 66944119 | 66786537 | GTGACAAAGATTGTTGTTTAAGAGTTGATT | |
| chrX: 66763874- | chrX: 66786538- | TTAGATTTTTTCCAAGTAAATGGTCAGCTGACTTGGAGCATCATCATTCCACTTGCTTTGAAAACCTGCCACTTAAGGCTCCTTCCAGTC | 278 |
| 66944119 | 66786657 | ATAGGTTAACTCTTTCTGGTCAAGTATtac | |
| chrX: 66763874- | chrX: 66786178- | tagTAAACATATGAACAGCAACTTGTGAAACGCCATTAGCAAAATCTCAAGTTATATTCTTCAGTGACTATGGCCATCCTAAAAATGGGG | 279 |
| 66944119 | 66786297 | TGTCTTTTATTTGGGGTAAATGAAGATGAA | |
| chrX: 66763874- | chrX: 66786238- | TCAGTGACTATGGCCATCCTAAAAATGGGGTGTCTTTTATTTGGGGTAAATGAAGATGAAGCCTTATGAGAAATTGCATTTTAATCTAAT | 280 |
| 66944119 | 66786357 | CTTGTCTTGCTAAGAACAGAAGTGGAATGT | |
| chrX: 66763874- | chrX: 66786478- | TCTGGTTATGTATTTGAGTAGAGTATGGGGGTGACAAAGATTGTTGTTTAAGAGTTGATTTTAGATTTTTTCCAAGTAAATGGTCAGCTG | 281 |
| 66944119 | 66786597 | ACTTGGAGCATCATCATTCCACTTGCTTTG | |
| chrX: 66763874- | chrX: 66786298- | GCCTTATGAGAAATTGCATTTTAATCTAATCTTGTCTTGCTAAGAACAGAAGTGGAATGTTTCAGCCTCTGTGTGTGTATTTGTGTGTGT | 282 |
| 66944119 | 66786417 | GAAGGTTGAGTGTGTGATGATGGATGGGGC | |
| chrX: 66763874- | chrX: 66787077- | CTCTTAAGTGCCTTATTTAACTGTAATATGGAAAATCAAAGTCACAGCTAATTCAGGAAAAATGAGTTTGGGATGTGAATTTCCTAGGCA | 283 |
| 66944119 | 66787196 | ACTTGTCATCTCTTTTTTACTTCCTTAGCT | |
| chrX: 66763874- | chrX: 66787197- | TCATAAACTTACCCACAATGTTCCCTGAGGACTAAGAGTAATGGAGGGTGATGAGGAAAGGCTTTCCTCCCTTCCTTTCCGAGAGTCCTT | 284 |
| 66944119 | 66787316 | TAGCCAAATGCCACACCTCCTCCTGTTTCC | |
| chrX: 66763874- | chrX: 66787317- | CTAGTCTCCGTGCAGAGATGGAAGTGGGAGATAGACATGGGTTCCTTTCAGCCCTGAGTTCATGCCAGGGTTTTCTTTCCCTCTAGCTGG | 285 |
| 66944119 | 66787436 | ACTGAGGTAGGAGGAGAGGTTGAAGTCCAC | |
| chrX: 66763874- | chrX: 66787257- | GCTTTCCTCCCTTCCTTTCCGAGAGTCCTTTAGCCAAATGCCACACCTCCTCCTGTTTCCCTAGTCTCCGTGCAGAGATGGAAGTGGGAG | 286 |
| 66944119 | 66787376 | ATAGACATGGGTTCCTTTCAGCCCTGAGTT | |
| chrX: 66763874- | chrX: 66786957- | TCCATCAGCCAAAGCCTCTTGCCTCCCTTCAACGTAACTCTTCTCTAGCGTCCTCTTAATAATCTTCTGAAAAGGTTTTACAGCCTTTCT | 287 |
| 66944119 | 66787076 | GGGTACTGGGACCCAGAGTCTTAATCCAGG | |
| chrX: 66763874- | chrX: 66787137- | AATGAGTTTGGGATGTGAATTTCCTAGGCAACTTGTCATCTCTTTTTTACTTCCTTAGCTTCATAAACTTACCCACAATGTTCCCTGAGG | 288 |
| 66944119 | 66787256 | ACTAAGAGTAATGGAGGGTGATGAGGAAAG | |
| chrX: 66763874- | chrX: 66787017- | AATCTTCTGAAAAGGTTTTACAGCCTTTCTGGGTACTGGGACCCAGAGTCTTAATCCAGGCTCTTAAGTGCCTTATTTAACTGTAATATG | 289 |
| 66944119 | 66787136 | GAAAATCAAAGTCACAGCTAATTCAGGAAA | |
| chrX: 66763874- | chrX: 66787689- | TACCACAAACTAAATCCTATACTTCAGGGATAAAATCTTCTCCTGTTTTTTCTAAAAGCCTGTGCATGTGTGGTGTAAGGGGTGGGTTTT | 290 |
| 66944119 | 66787808 | CCCTTGTACCAGCAACTTAGCAATTGTAGT | |
| chrX: 66763874- | chrX: 66787629- | TCTAGAAGTAATTTGTAAATTCAGAAAGGGCTTATAGATTTAAAGTGTAGCCGTTTTGATTACCACAAACTAAATCCTATACTTCAGGGA | 291 |
| 66944119 | 66787748 | TAAAATCTTCTCCTGTTTTTTCTAAAAGCC | |
| chrX: 66763874- | chrX: 66787809- | AACGGGGCTGAGGGCAGTGGCATGCTTCTTCATTGAGCAAGTGTGAAAAGAGGGTTATGCATTCAGGGGTCAGCAGATGGCAGGCAGAGT | 292 |
| 66944119 | 66787928 | AGCCCCTCCAAATCTCCCTCCCATACCACA | |
| chrX: 66763874- | chrX: 66787749- | TGTGCATGTGTGGTGTAAGGGGTGGGTTTTCCCTTGTACCAGCAACTTAGCAATTGTAGTAACGGGGCTGAGGGCAGTGGCATGCTTCTT | 293 |
| 66944119 | 66787868 | CATTGAGCAAGTGTGAAAAGAGGGTTATGC | |
| chrX: 66763874- | chrX: 66787869- | ATTCAGGGGTCAGCAGATGGCAGGCAGAGTAGCCCCTCCAAATCTCCCTCCCATACCACAAAGCCCTCTTATTTATTCAAACTTAACATT | 294 |
| 66944119 | 66787988 | AGAAGCTCATTTCAAGTAGGCACGTCTGTG | |
| chrX: 66763874- | chrX: 66787929- | AAGCCCTCTTATTTATTCAAACTTAACATTAGAAGCTCATTTCAAGTAGGCACGTCTGTGTCTGGGCGTCTATTTTCCTTCTTTGTATAT | 295 |
| 66944119 | 66788048 | AGCAGGCATTTGTCAACTTGGTGAAAAGCA | |
| chrX: 66763874- | chrX: 66787989- | TCTGGGCGTCTATTTTCCTTCTTTGTATATAGCAGGCATTTGTCAACTTGGTGAAAAGCATTACTCTTCTTTCCATTTCTGAGGACTAAT | 296 |
| 66944119 | 66788108 | TGTGCTTCTTCGCTAGACACGAGTTCAAAA | |
| chrX: 66763874- | chrX: 66789775- | CTTATCTTAGTTTGTATTTCTCTGGGTGTATATCCCTCTCTTGCAGTTCTGGGCCTTTGCAGTTTTTGGCTTATGTTTTTGTATATATCC | 297 |
| 66944119 | 66789894 | ACTAGAATTGGCTTCTTATCTTTTTTGTGC | |
| chrX: 66763874- | chrX: 66790495- | TTGTACTGCAAAGTCTTATTTCTGATAAGACATCACAAATAAGAATTATTGTGATGAGACTTATCACAAATAAGAATTATTGTGATAATT | 298 |
| 66944119 | 66790614 | CTTATTTGTGATAAGAATTACTGGGTTAGA | |
| chrX: 66763874- | chrX: 66789355- | CCTGTGTGCTTTGCTAGTGGGAACGTGTCTTTTCTTTTGTGCTCGTATCTCTGTGTAATCGAGTGTCTTGCTAAGTCAATGTGCCTCTGT | 299 |
| 66944119 | 66789474 | CTCTTTTTACCAGTTCTGTCTTTGTGTCTC | |
| chrX: 66763874- | chrX: 66790975- | AACTTCTGTTCTTTTGGTTTCTCTTTCTTTCTTTGATTATATTATTTTCTGGACTTGTTCTGCCAAAGCAAGAAGGAAATTCCACATGTG | 300 |
| 66944119 | 66791094 | GCTCACTCATTTATTATACTTGTTTCTTTG | |
| chrX: 66763874- | chrX: 66789475- | TGTGCCTTCATGTATTTTTTCCCCTGAGTTTGCACGTCTCTGTCTATGTGGATATCTCTCACTCCAGGCCACTGTATCACTGTGTCTGTA | 301 |
| 66944119 | 66789594 | TTACAGCTGTTTATTTCTGTCGGTGTGTGG | |
| chrX: 66763874- | chrX: 66789055- | GGGGGGGGGTTGGGGGTTGAGCAATTCATTATTATTAAAATGCAAAAAGCACTTAATTCGCTATGATAAGATTGCCTTTTTCATGCATAC | 302 |
| 66944119 | 66789174 | TGGCCTACCTGCAAGACCCCTAGAGACAGT | |
| chrX: 66763874- | chrX: 66789655- | ATTTCAGTTTATGTCATAGCCATTCTTTGTGTGACTGCTTCTAGGTATGTCTTTTTCTATGCCCCTATTGTCCCCATCTCCATGTGTCTC | 303 |
| 66944119 | 66789774 | TGTGTGTATATGTTCTAATGTATCTGCCTA | |
| chrX: 66763874- | chrX: 66789235- | CTGCACATGTGTTGTGGTTTGATGTTTGTATGTGATTGTGTACCAGGGTATGTGTGTCTGTTATTGTGAGTTCATTTCTGAGCAGTTGTG | 304 |
| 66944119 | 66789354 | ACACACAGAGATCCAGAAACAGTGTCTTAC | |
| chrX: 66763874- | chrX: 66790795- | GGATGGAGTGCACAGGAAATGGAGAGGTGAAGGTCATAGAGAGAAGTTTAGCAGGACCAGATCTTTCCTTGTCCTGGGCTGCTGTGACCA | 305 |
| 66944119 | 66790914 | TATAAGGAAGGCAGTAAGGGGAGGGGTAGG | |
| chrX: 66763874- | chrX: 66790315- | AGTGTGTCTTTATTAACAGGACACTTGTGTGTAGAGAATCCTTGAGAAATGAGTGGTTAGATGATAAATCTTTTCATATTAATTTCATGA | 306 |
| 66944119 | 66790434 | TGTCAGTGAAGTAAATTTGCAAGATATGGG | |
| chrX: 66763874- | chrX: 66791635- | GATTTAGTTAGAATTATTGAATGTAAAATAATGAAAATTAAAAAAAAAACAAGGAGGAGGAATCTATCCTATTTTATAATTCAGACCGTT | 307 |
| 66944119 | 66791754 | GAATTGAGTTTTTCTTTTGTTGTATTGATT | |
| chrX: 66763874- | chrX: 66791335- | TGGTCATTAATAAAAGACTAGGTTCAGTAGAAACATGTAAGTTGTCTAGGTGTTGGAAATTAATACAGTACTGTGCTAAGGGAACATATA | 308 |
| 66944119 | 66791454 | TCTAGAAGTTAACTGAATTATGCTCAATAA | |
| chrX: 66763874- | chrX: 66788575- | GTATGTTAGTTTGCTTATCTGCGCCGTCCCTCCTCTTCCCAACCCACTGTGTATTGCAGAATGTTTTATCAGCTCTGATTTGCCAAGTTG | 309 |
| 66944119 | 66788694 | CTCTCTTCTCCAGTAGGTGCTGCGAGCAGA | |
| chrX: 66763874- | chrX: 66790015- | ATGTGTACGCATCCATGTATCTTTCTGCTTCTCTGTGACCAGATATTTCTGTGTAGCTGTCTATGTATATTGGCTTCTGTCTGTGTCTGT | 310 |
| 66944119 | 66790134 | GTTGTTGGCTCTACGTCTGTGCATATGCAC | |
| chrX: 66763874- | chrX: 66789595- | ATTTCTATGTCTGTTTTCATCTTAATTTGTGTGTCTAAGCAAGACTGTTTTGGGGTGACTATTTCAGTTTATGTCATAGCCATTCTTTGT | 311 |
| 66944119 | 66789714 | GTGACTGCTTCTAGGTATGTCTTTTTCTAT | |
| chrX: 66763874- | chrX: 66791215- | AATGGGCGCAACCATGATTTTTCTGATGTCAGTCTGTGATGTCAGTTGTCCAGTGTGTATGCAGGCTGCTTAAGAGTACATACAGTTCCT | 312 |
| 66944119 | 66791334 | TCACAATTATGGTAGTCCCTGAGAAGGAAG | |
| chrX: 66763874- | chrX: 66790435- | CTGCATAAGAACTATGTTCTTTTTAAAACTCAGCATATTGATGGTGGAGAAAGCATTTATTTGTACTGCAAAGTCTTATTTCTGATAAGA | 313 |
| 66944119 | 66790554 | CATCACAAATAAGAATTATTGTGATGAGAC | |
| chrX: 66763874- | chrX: 66788635- | ATGTTTTATCAGCTCTGATTTGCCAAGTTGCTCTCTTCTCCAGTAGGTGCTGCGAGCAGAGAGGGATTCCTCGGAGGTCATCTGTTCCAT | 314 |
| 66944119 | 66788754 | CTTCTTGCCTATGCAAATGCCTGCCTGAAG | |
| chrX: 66763874- | chrX: 66790255- | CGTAGGTGTGCATAATTTATTCATGTAGGATGTCAAAAGAGTCAGTTAAAAATTATGCACAGTGTGTCTTTATTAACAGGACACTTGTGT | 315 |
| 66944119 | 66790374 | GTAGAGAATCCTTGAGAAATGAGTGGTTAG | |
| chrX: 66763874- | chrX: 66791155- | ATATGGGCAGGGGGTTCTTTATGCCAGTTCTGCTCTCTTCCCAGTGTATCTGTGGTGCTTAATGGGCGCAACCATGATTTTTCTGATGTC | 316 |
| 66944119 | 66791274 | AGTCTGTGATGTCAGTTGTCCAGTGTGTAT | |
| chrX: 66763874- | chrX: 66791095- | CACGATATTAAAGACAGCTTGTTAAGTGTCACTGCAAACATCATACACACTGATCCACTGATATGGGCAGGGGGTTCTTTATGCCAGTTC | 317 |
| 66944119 | 66791214 | TGCTCTCTTCCCAGTGTATCTGTGGTGCTT | |
| chrX: 66763874- | chrX: 66788875- | CTTCAATTGAAAACTTAGAACTCAGTTTCTAGGGTAGTGAGTGTTGTAAGGTTTGGACTGTGACCTAATATTACGCAGCCATGACATTAT | 318 |
| 66944119 | 66788994 | CTATTAGGCATCTAGACTAGCTTGCTTGAA | |
| chrX: 66763874- | chrX: 66789115- | CTATGATAAGATTGCCTTTTTCATGCATACTGGCCTACCTGCAAGACCCCTAGAGACAGTAAGCAGCATACATGGTGTCTTCCAGTTTTC | 319 |
| 66944119 | 66789234 | AGCCTTTGTGCAAGGAACAACTGTGGGTTT | |
| chrX: 66763874- | chrX: 66788455- | TCAGAGGCCTGGCAGTCAGAGGGATTCTGATCTCTATATGCAATATTTTCACACTACTGTACTTATTGAAATCACATTTGAATCTTGGCA | 320 |
| 66944119 | 66788574 | ATTAACAAGGCAGTAATTGGCATCAGGAGG | |
| chrX: 66763874- | chrX: 66789535- | ACTCCAGGCCACTGTATCACTGTGTCTGTATTACAGCTGTTTATTTCTGTCGGTGTGTGGATTTCTATGTCTGTTTTCATCTTAATTTGT | 321 |
| 66944119 | 66789654 | GTGTCTAAGCAAGACTGTTTTGGGGTGACT | |
| chrX: 66763874- | chrX: 66791575- | TAAAATTGACATGACAAAATTAAGTCATTGTGTCTGTTTTATGATAAAACAGGCTCTTTTGATTTAGTTAGAATTATTGAATGTAAAATA | 322 |
| 66944119 | 66791694 | ATGAAAATTAAAAAAAAAACAAGGAGGAGG | |
| chrX: 66763874- | chrX: 66791275- | GCAGGCTGCTTAAGAGTACATACAGTTCCTTCACAATTATGGTAGTCCCTGAGAAGGAAGTGGTCATTAATAAAAGACTAGGTTCAGTAG | 323 |
| 66944119 | 66791394 | AAACATGTAAGTTGTCTAGGTGTTGGAAAT | |
| chrX: 66763874- | chrX: 66791695- | AATCTATCCTATTTTATAATTCAGACCGTTGAATTGAGTTTTTCTTTTGTTGTATTGATTTAAATGCAGAGAAGTCTATGATGCTGGATT | 324 |
| 66944119 | 66791814 | CCAGTCAGAAGATAAACATTTGTATGTGGG | |
| chrX: 66763874- | chrX: 66791455- | AAAGAGTACAAATGTTTCATAAATATTTTGACCTAATCCTCCTGTAAGATTAGGAGAGGGATATTTCCGATATTCAAATAATTTTTTTAA | 325 |
| 66944119 | 66791574 | TTGGCAAACACCTTAGACATACTATTTACA | |
| chrX: 66763874- | chrX: 66789955- | TGTTTGCAGTCAGTTGTGTTGGCTGTCCCTGTGTTTGTCCCTGTGTGTGCATTTCATTGTATGTGTACGCATCCATGTATCTTTCTGCTT | 326 |
| 66944119 | 66790074 | CTCTGTGACCAGATATTTCTGTGTAGCTGT | |
| chrX: 66763874- | chrX: 66790915- | GATGAGGAAGAGACCAGCTCTCCTCTTTCTTTCTGATGGAAGGTTACCACCTCTATTTAAAACTTCTGTTCTTTTGGTTTCTCTTTCTTT | 327 |
| 66944119 | 66791034 | CTTTGATTATATTATTTTCTGGACTTGTTC | |
| chrX: 66763874- | chrX: 66789715- | GCCCCTATTGTCCCCATCTCCATGTGTCTCTGTGTGTATATGTTCTAATGTATCTGCCTACTTATCTTAGTTTGTATTTCTCTGGGTGTA | 328 |
| 66944119 | 66789834 | TATCCCTCTCTTGCAGTTCTGGGCCTTTGC | |
| chrX: 66763874- | chrX: 66789835- | AGTTTTTGGCTTATGTTTTTGTATATATCCACTAGAATTGGCTTCTTATCTTTTTTGTGCATGTTTTAGTTTGTATGAGTGAGCATATCC | 329 |
| 66944119 | 66789954 | AACTCTGTCTTTGAGAAGCAGAACTGTCTG | |
| chrX: 66763874- | chrX: 66791035- | TGCCAAAGCAAGAAGGAAATTCCACATGTGGCTCACTCATTTATTATACTTGTTTCTTTGCACGATATTAAAGACAGCTTGTTAAGTGTC | 330 |
| 66944119 | 66791154 | ACTGCAAACATCATACACACTGATCCACTG | |
| chrX: 66763874- | chrX: 66790075- | CTATGTATATTGGCTTCTGTCTGTGTCTGTGTTGTTGGCTCTACGTCTGTGCATATGCACCCACCGGGTTCATAAAAAGCTCACCTGCTC | 331 |
| 66944119 | 66790194 | TCCAAGGAATCTACCAGATTATTTTGTGAA | |
| chrX: 66763874- | chrX: 66791395- | TAATACAGTACTGTGCTAAGGGAACATATATCTAGAAGTTAACTGAATTATGCTCAATAAAAAGAGTACAAATGTTTCATAAATATTTTG | 332 |
| 66944119 | 66791514 | ACCTAATCCTCCTGTAAGATTAGGAGAGGG | |
| chrX: 66763874- | chrX: 66789895- | ATGTTTTAGTTTGTATGAGTGAGCATATCCAACTCTGTCTTTGAGAAGCAGAACTGTCTGTGTTTGCAGTCAGTTGTGTTGGCTGTCCCT | 333 |
| 66944119 | 66790014 | GTGTTTGTCCCTGTGTGTGCATTTCATTGT | |
| chrX: 66763874- | chrX: 66788815- | TGCTCCTGACATTGCCTGTCACTTTTTCCCATGATACTCTGGCTTCACAGGTGGGAGGTTCTTCAATTGAAAACTTAGAACTCAGTTTCT | 334 |
| 66944119 | 66788934 | AGGGTAGTGAGTGTTGTAAGGTTTGGACTG | |
| chrX: 66763874- | chrX: 66788995- | TATCTTAGCATGTTGACTAATTTGGGGCAGAATATAGTGTGGGTGGGGGATTTTGTGTGTGGGGGGGGGTTGGGGGTTGAGCAATTCATT | 335 |
| 66944119 | 66789114 | ATTATTAAAATGCAAAAAGCACTTAATTCG | |
| chrX: 66763874- | chrX: 66788755- | CTGCTGGAGGCTGGCTTTGTACCGGACTTTGTACAGGGAACCAGGGAAACGAATGCAGAGTGCTCCTGACATTGCCTGTCACTTTTTCCC | 336 |
| 66944119 | 66788874 | ATGATACTCTGGCTTCACAGGTGGGAGGTT | |
| chrX: 66763874- | chrX: 66788935- | TGACCTAATATTACGCAGCCATGACATTATCTATTAGGCATCTAGACTAGCTTGCTTGAATATCTTAGCATGTTGACTAATTTGGGGCAG | 337 |
| 66944119 | 66789054 | AATATAGTGTGGGTGGGGGATTTTGTGTGT | |
| chrX: 66763874- | chrX: 66790855- | ATCTTTCCTTGTCCTGGGCTGCTGTGACCATATAAGGAAGGCAGTAAGGGGAGGGGTAGGGATGAGGAAGAGACCAGCTCTCCTCTTTCT | 338 |
| 66944119 | 66790974 | TTCTGATGGAAGGTTACCACCTCTATTTAA | |
| chrX: 66763874- | chrX: 66790735- | ATGAAAAGTAACAGAGAGATGGGCATATTCCTTGTTTGAATGGAGTCATCCAGGGGCTCAGGATGGAGTGCACAGGAAATGGAGAGGTGA | 339 |
| 66944119 | 66790854 | AGGTCATAGAGAGAAGTTTAGCAGGACCAG | |
| chrX: 66763874- | chrX: 66788515- | ACTTATTGAAATCACATTTGAATCTTGGCAATTAACAAGGCAGTAATTGGCATCAGGAGGGTATGTTAGTTTGCTTATCTGCGCCGTCCC | 340 |
| 66944119 | 66788634 | TCCTCTTCCCAACCCACTGTGTATTGCAGA | |
| chrX: 66763874- | chrX: 66790135- | CCACCGGGTTCATAAAAAGCTCACCTGCTCTCCAAGGAATCTACCAGATTATTTTGTGAAATAACTCACGTTTCGTTTTTTTACTTGCCA | 341 |
| 66944119 | 66790254 | GCTGCTATGGTACTTAAAAGTGTGTTGGTA | |
| chrX: 66763874- | chrX: 66789175- | AAGCAGCATACATGGTGTCTTCCAGTTTTCAGCCTTTGTGCAAGGAACAACTGTGGGTTTCTGCACATGTGTTGTGGTTTGATGTTTGTA | 342 |
| 66944119 | 66789294 | TGTGATTGTGTACCAGGGTATGTGTGTCTG | |
| chrX: 66763874- | chrX: 66791515- | ATATTTCCGATATTCAAATAATTTTTTTAATTGGCAAACACCTTAGACATACTATTTACATAAAATTGACATGACAAAATTAAGTCATTG | 343 |
| 66944119 | 66791634 | TGTCTGTTTTATGATAAAACAGGCTCTTTT | |
| chrX: 66763874- | chrX: 66788335- | TGTTCTTTGATCTCAGTGATTTTGACTCTTTCTACTGCACTCTGGGGACAGTGGGTTCTGCGGTACCAACTCCAATTAAAGTGGGAATAT | 344 |
| 66944119 | 66788454 | GTACCAGCCCCTCCCCTTGGTTTTTATTTT | |
| chrX: 66763874- | chrX: 66789415- | GAGTGTCTTGCTAAGTCAATGTGCCTCTGTCTCTTTTTACCAGTTCTGTCTTTGTGTCTCTGTGCCTTCATGTATTTTTTCCCCTGAGTT | 345 |
| 66944119 | 66789534 | TGCACGTCTCTGTCTATGTGGATATCTCTC | |
| chrX: 66763874- | chrX: 66790615- | AGGTGTTACTTTTCTGGTTTTGTTTGGGTTTTTGTTTTGAAGTGTTACTACAGATGGTGTCTTAGGGACAAAGAGCTCTGAGGTTGACTT | 346 |
| 66944119 | 66790734 | AGAACACATGGAGTACAGATAAAAAGGAGA | |
| chrX: 66763874- | chrX: 66790195- | ATAACTCACGTTTCGTTTTTTTACTTGCCAGCTGCTATGGTACTTAAAAGTGTGTTGGTACGTAGGTGTGCATAATTTATTCATGTAGGA | 347 |
| 66944119 | 66790314 | TGTCAAAAGAGTCAGTTAAAAATTATGCAC | |
| chrX: 66763874- | chrX: 66790375- | ATGATAAATCTTTTCATATTAATTTCATGATGTCAGTGAAGTAAATTTGCAAGATATGGGCTGCATAAGAACTATGTTCTTTTTAAAACT | 348 |
| 66944119 | 66790494 | CAGCATATTGATGGTGGAGAAAGCATTTAT | |
| chrX: 66763874- | chrX: 66790675- | CTTAGGGACAAAGAGCTCTGAGGTTGACTTAGAACACATGGAGTACAGATAAAAAGGAGAATGAAAAGTAACAGAGAGATGGGCATATTC | 349 |
| 66944119 | 66790794 | CTTGTTTGAATGGAGTCATCCAGGGGCTCA | |
| chrX: 66763874- | chrX: 66788395- | CGGTACCAACTCCAATTAAAGTGGGAATATGTACCAGCCCCTCCCCTTGGTTTTTATTTTTCAGAGGCCTGGCAGTCAGAGGGATTCTGA | 350 |
| 66944119 | 66788514 | TCTCTATATGCAATATTTTCACACTACTGT | |
| chrX: 66763874- | chrX: 66789295- | TTATTGTGAGTTCATTTCTGAGCAGTTGTGACACACAGAGATCCAGAAACAGTGTCTTACCCTGTGTGCTTTGCTAGTGGGAACGTGTCT | 351 |
| 66944119 | 66789414 | TTTCTTTTGTGCTCGTATCTCTGTGTAATC | |
| chrX: 66763874- | chrX: 66788695- | GAGGGATTCCTCGGAGGTCATCTGTTCCATCTTCTTGCCTATGCAAATGCCTGCCTGAAGCTGCTGGAGGCTGGCTTTGTACCGGACTTT | 352 |
| 66944119 | 66788814 | GTACAGGGAACCAGGGAAACGAATGCAGAG | |
| chrX: 66763874- | chrX: 66790555- | TTATCACAAATAAGAATTATTGTGATAATTCTTATTTGTGATAAGAATTACTGGGTTAGAAGGTGTTACTTTTCTGGTTTTGTTTGGGTT | 353 |
| 66944119 | 66790674 | TTTGTTTTGAAGTGTTACTACAGATGGTGT | |
| chrX: 66763874- | chrX: 66792161- | ATCCGTGGTAAAAATTATTAATGCTTTGCACATGCAACATAGAGTGTTCAATTTTGTTAGTCAACAAATATTTAAGTGGCAGCTGTTATG | 354 |
| 66944119 | 66792280 | ACCTCAGGGGTGTAGTGACTTCCTTATTGT | |
| chrX: 66763874- | chrX: 66792641- | TGGGTAGTCAAAAATAGTTACATATACAAGTCAGCATTTTTTAAATTGTTCAGTTGTGCTTAAGATTGGTCCTTTCCAGGAACAATCCAG | 355 |
| 66944119 | 66792760 | CTTTATCAAAAAATTATTGCGTACATGTAA | |
| chrX: 66763874- | chrX: 66792581- | TGAGATTACTTCTTTTCAGGTAGGAAACTCTACTGTGTATTTGGCTAGTTCAACCTATCATGGGTAGTCAAAAATAGTTACATATACAAG | 356 |
| 66944119 | 66792700 | TCAGCATTTTTTAAATTGTTCAGTTGTGCT | |
| chrX: 66763874- | chrX: 66792281- | CCTTTAATTATTAAAAAAGAAATCTATATCAGAATATCAGGTAAACTCTTATTACATCAAATATTATAATAAAGATACTTTTTATATTCT | 357 |
| 66944119 | 66792400 | CTAAACAAAGTAGAGATCTCAGATGTTGGT | |
| chrX: 66763874- | chrX: 66792401- | TCATTTATCAATATAATATTAGATTTGAAAATTCCAGTATACAAAAGGAAAAGGACAGCTTCTTAAAGTTTATAGTGATTTTCTATGAAC | 358 |
| 66944119 | 66792520 | TATCAATTCCGTTTTTTTCTGTTTTACTGG | |
| chrX: 66763874- | chrX: 66792041- | GCAAACCCTGGCCATTCTGTTTTGTTTAGGAAAGAATTCATCAGTTCTGATTCTGCCTTTTCTGGGGAGGGAGGCTGAGTATTGGATTGA | 359 |
| 66944119 | 66792160 | AGAGGAGTCACTACTTTTCTGAGATGATAT | |
| chrX: 66763874- | chrX: 66792341- | ATATTATAATAAAGATACTTTTTATATTCTCTAAACAAAGTAGAGATCTCAGATGTTGGTTCATTTATCAATATAATATTAGATTTGAAA | 360 |
| 66944119 | 66792460 | ATTCCAGTATACAAAAGGAAAAGGACAGCT | |
| chrX: 66763874- | chrX: 66792101- | TCTGGGGAGGGAGGCTGAGTATTGGATTGAAGAGGAGTCACTACTTTTCTGAGATGATATATCCGTGGTAAAAATTATTAATGCTTTGCA | 361 |
| 66944119 | 66792220 | CATGCAACATAGAGTGTTCAATTTTGTTAG | |
| chrX: 66763874- | chrX: 66792461- | TCTTAAAGTTTATAGTGATTTTCTATGAACTATCAATTCCGTTTTTTTCTGTTTTACTGGTATGATGGAAACTAAATTTCGAGTTGTAAG | 362 |
| 66944119 | 66792580 | TAGTAGATAATTAGACTGCAGGGTAAGCCT | |
| chrX: 66763874- | chrX: 66792521- | TATGATGGAAACTAAATTTCGAGTTGTAAGTAGTAGATAATTAGACTGCAGGGTAAGCCTTGAGATTACTTCTTTTCAGGTAGGAAACTC | 363 |
| 66944119 | 66792640 | TACTGTGTATTTGGCTAGTTCAACCTATCA | |
| chrX: 66763874- | chrX: 66792221- | TCAACAAATATTTAAGTGGCAGCTGTTATGACCTCAGGGGTGTAGTGACTTCCTTATTGTCCTTTAATTATTAAAAAAGAAATCTATATC | 364 |
| 66944119 | 66792340 | AGAATATCAGGTAAACTCTTATTACATCAA | |
| chrX: 66763874- | chrX: 66793277- | ACGTTTATCCATTTAGGGACAGCAGGTTTGGCACAAATGGATTGGTTTTCTGAGGTCTTATGTAGAGGGCTGCACTGACTGACTTCTGAA | 365 |
| 66944119 | 66793396 | AGTCCCCCCTAACCCTTCAAATCTCAGGGT | |
| chrX: 66763874- | chrX: 66793157- | GCTTTGAGTTAGAAAGATAGAGTAAGATGGAGGAACCAATTCTTCCCTGGGTTGATATTTATTTATCTTGCTCTTTTGAAGTCTAGGCCA | 366 |
| 66944119 | 66793276 | ATCATCCTATTTATTCTGAATGGCCCGTTA | |
| chrX: 66763874- | chrX: 66793037- | AAGCTCTACACTTTTAGAGGGCCATTAACAATGCTCAAGTTAAAGAAAAGCAATCAAAGACAACTAAAATACTGGTACCTTCAAACAGTA | 367 |
| 66944119 | 66793156 | CTTATGAATTATTTAACCTTAGATAATTTG | |
| chrX: 66763874- | chrX: 66793217- | ATTTATCTTGCTCTTTTGAAGTCTAGGCCAATCATCCTATTTATTCTGAATGGCCCGTTAACGTTTATCCATTTAGGGACAGCAGGTTTG | 368 |
| 66944119 | 66793336 | GCACAAATGGATTGGTTTTCTGAGGTCTTA | |
| chrX: 66763874- | chrX: 66793397- | CATCTGGTCTCAAGCCTTCAATTATGAATACATTTCTATTGCCTTTTTGAGTAACAGCACAACACTGCAAGCTGACCCACTGGGTGGATG | 369 |
| 66944119 | 66793516 | GAATGGGGCTCTTGCCCTACCACCCTTTGG | |
| chrX: 66763874- | chrX: 66792917- | CATAGTTATTTCCCTATAATATTTGTTTTATGATTGTATAGATGTCTGCTGACCAACCTTAATCTCTGCTCCCTAAGATTAACCATTCTA | 370 |
| 66944119 | 66793036 | CAAAGCAGAAACTGGAGGTCATTCAAATGA | |
| chrX: 66763874- | chrX: 66793097- | CAACTAAAATACTGGTACCTTCAAACAGTACTTATGAATTATTTAACCTTAGATAATTTGGCTTTGAGTTAGAAAGATAGAGTAAGATGG | 371 |
| 66944119 | 66793216 | AGGAACCAATTCTTCCCTGGGTTGATATTT | |
| chrX: 66763874- | chrX: 66793337- | TGTAGAGGGCTGCACTGACTGACTTCTGAAAGTCCCCCCTAACCCTTCAAATCTCAGGGTCATCTGGTCTCAAGCCTTCAATTATGAATA | 372 |
| 66944119 | 66793456 | CATTTCTATTGCCTTTTTGAGTAACAGCAC | |
| chrX: 66763874- | chrX: 66792977- | AATCTCTGCTCCCTAAGATTAACCATTCTACAAAGCAGAAACTGGAGGTCATTCAAATGAAAGCTCTACACTTTTAGAGGGCCATTAACA | 373 |
| 66944119 | 66793096 | ATGCTCAAGTTAAAGAAAAGCAATCAAAGA | |
| chrX: 66763874- | chrX: 66795950- | CTTCGAATTTTTGTCAAAAAGTATTCTTTCATTAGAAAGATACATGGGTGTGCTTCCATGTCAGCAACATGACTGCAGACCAGGAAGTCC | 374 |
| 66944119 | 66796069 | TCACGGAGAGCTGGAATATGGGTATTTTGG | |
| chrX: 66763874- | chrX: 66794150- | CTCCCACCCTCTAAAACAACAGAGGCAGCAACCATTTACACACTTTCCAGAAGTAAGTAAGTAAGACTGTATTCCAGAAACACCCTATAT | 375 |
| 66944119 | 66794269 | CAAAATGGAAATATACTCAAGTGCCCCAAT | |
| chrX: 66763874- | chrX: 66794210- | GTAAGACTGTATTCCAGAAACACCCTATATCAAAATGGAAATATACTCAAGTGCCCCAATGACCCATTGGGCTAGTTTGAACGTGTGCAG | 376 |
| 66944119 | 66794329 | TCTCTGTGCTCCCCGTTTTAGCTTAAGCCT | |
| chrX: 66763874- | chrX: 66794990- | ACTGAGCAGTGGGAGTGGAGCAGGTTGGGGATAGTGAAGTATTTGTAATTCATTTTTAAAAAGGAGAGGGAGAGAGAAAAGGAAAAACTG | 377 |
| 66944119 | 66795109 | GGCCACCCATCCTTTGAAAAGAAACCTTGA | |
| chrX: 66763874- | chrX: 66795590- | TAGCTTGCTAGTAACTTCACATGCTATTTCCTTTACCTCTTATATTTGAGGTGTCTATTTGGAGTGGGCTGTGTTTCTAGCTATTCTGTT | 378 |
| 66944119 | 66795709 | TATCTGGTTTGTTTTTGTTGGTGTAGGAAA | |
| chrX: 66763874- | chrX: 66795830- | TTATGAGAAAAAAAGTCAAACAAACATATTTGAAATGTCCAGAAAACCTGTGAGTTTTTATGTATACTATACAGGAAAGATATTCTGTCA | 379 |
| 66944119 | 66795949 | TCTGGTTGCCAAACTATGGAGGGTGGGAGA | |
| chrX: 66763874- | chrX: 66796610- | TTGCTGGTGGATATGGTGGAATACCTTTTATGTGGTTATCTCCTCCTTGTAACTCTTGGCTGCATAACCCTTATTTTCTTTTCTATTTTT | 380 |
| 66944119 | 66796729 | ATTCTCTCTCTTGGAAAAAAAATTGGTGGT | |
| chrX: 66763874- | chrX: 66794030- | AGAGGGTAGTTTGAAAGAAAATGCTAGTGACTACGTGTGTTTCCTTCCTGACATATTTTATAGAAGGTGATGAGTTCCAGCATTTTTTCA | 381 |
| 66944119 | 66794149 | GACTTGGATCTGGCTTTCATTCCCCTTCTC | |
| chrX: 66763874- | chrX: 66794870- | CTTTGTAGGACATATGATCTTTGCTAAGTGCACTGAATGTATGTAGAGGAGACAAGTCTGCTGAGGGTATGAGAATTGGGCCAAGATTTA | 382 |
| 66944119 | 66794989 | ACACATTTTCAAAGCTCCATGAAGAAGCCT | |
| chrX: 66763874- | chrX: 66795350- | GGGATAGCATTAAAAGAAATAGTGCTTTTGTTTAGAAGAAGAAATGAAATGCTTGTGTCCAGATGCTTAAAGGAAGGCAGTGCAGACTTT | 383 |
| 66944119 | 66795469 | CAGAAACTAGACTTTAAGAGCTGTACTCAG | |
| chrX: 66763874- | chrX: 66796070- | ACTCTCTGGTTAGATGCAGCTTTTACTTCACATCCTCAGTGGTACTACTGTAAATTTTCATTTTCCTGTGGAATACCCTATTTGGTTCCA | 384 |
| 66944119 | 66796189 | TTGTATATAGTTGACAACTAGAATTCGTTC | |
| chrX: 66763874- | chrX: 66794930- | CTGAGGGTATGAGAATTGGGCCAAGATTTAACACATTTTCAAAGCTCCATGAAGAAGCCTACTGAGCAGTGGGAGTGGAGCAGGTTGGGG | 385 |
| 66944119 | 66795049 | ATAGTGAAGTATTTGTAATTCATTTTTAAA | |
| chrX: 66763874- | chrX: 66794690- | AGCGGCATTTTTTTACTTCTCAATATGAGGTTGAAACTATAAGCTTAAATTGCTGACTTTCTGGCAGCACCAAACAGTAAGGAAACCACA | 386 |
| 66944119 | 66794809 | AAGATAAACCCAAATAATAGAGCCAATTTT | |
| chrX: 66763874- | chrX: 66794090- | TAGAAGGTGATGAGTTCCAGCATTTTTTCAGACTTGGATCTGGCTTTCATTCCCCTTCTCCTCCCACCCTCTAAAACAACAGAGGCAGCA | 387 |
| 66944119 | 66794209 | ACCATTTACACACTTTCCAGAAGTAAGTAA | |
| chrX: 66763874- | chrX: 66794570- | GCTACAATTAATCCAAGCAAAGGGAAAGATGTCAGTAAAACTGCCCCTTTTCATAGAGGTGTGGCAACTGCTGGGAAGGAAGAAATTAGC | 388 |
| 66944119 | 66794689 | CTGAGGCCATGTGATTACTAATAAACTCAA | |
| chrX: 66763874- | chrX: 66796250- | GGAAGAGCTACCATAATGAATGTGTACATGGACAAAAAAAAAGAGAGAGAGAGAGAGAATTAAATCATGAGTTTGTGCCTTGGGAGCTAC | 389 |
| 66944119 | 66796369 | AGTTTAAACATTTGCTGTTTTTCTCACTTA | |
| chrX: 66763874- | chrX: 66796490- | TTTAGTAAAATGGCCACCAGAAATAAAGGATTTTATTTTCCAGACTTTGGTGTTTGGAGCTGGTGTGCTGAGAGCTAGCAGAGAAAGCCC | 390 |
| 66944119 | 66796609 | TACTCAGGTAGATGTACCAGAGCAGGATGG | |
| chrX: 66763874- | chrX: 66796550- | TGGTGTGCTGAGAGCTAGCAGAGAAAGCCCTACTCAGGTAGATGTACCAGAGCAGGATGGTTGCTGGTGGATATGGTGGAATACCTTTTA | 391 |
| 66944119 | 66796669 | TGTGGTTATCTCCTCCTTGTAACTCTTGGC | |
| chrX: 66763874- | chrX: 66794810- | CTTTTTTTCCGGGGGGGATGACTTCTAACTAGTGATATGAGGAAGGATAAGAAAATGTTTCTTTGTAGGACATATGATCTTTGCTAAGTG | 392 |
| 66944119 | 66794929 | CACTGAATGTATGTAGAGGAGACAAGTCTG | |
| chrX: 66763874- | chrX: 66796190- | GCTGTTGCTTGAGCCCAACTATAACTTCTTGGCACTATACCTATCTTCTGATGTGCCTGTGGAAGAGCTACCATAATGAATGTGTACATG | 393 |
| 66944119 | 66796309 | GACAAAAAAAAAGAGAGAGAGAGAGAGAAT | |
| chrX: 66763874- | chrX: 66794630- | GTGGCAACTGCTGGGAAGGAAGAAATTAGCCTGAGGCCATGTGATTACTAATAAACTCAAAGCGGCATTTTTTTACTTCTCAATATGAGG | 394 |
| 66944119 | 66794749 | TTGAAACTATAAGCTTAAATTGCTGACTTT | |
| chrX: 66763874- | chrX: 66795230- | AGAAATAATTGTAGGTAGCTTAGCCTTGGCTGTAGTCAGAACTTTTGTACTGTGACTTTAGGATCTGTATGGAATCGTATGATATGCGGA | 395 |
| 66944119 | 66795349 | TACACCAAAAACTCTATGGGTTATCAAAAT | |
| chrX: 66763874- | chrX: 66795650- | GGAGTGGGCTGTGTTTCTAGCTATTCTGTTTATCTGGTTTGTTTTTGTTGGTGTAGGAAACTGGTATAAATTTTATTTGGGTAAATATCA | 396 |
| 66944119 | 66795769 | CCTCAATTTTCAACTAAAGCTTTATTTAAG | |
| chrX: 66763874- | chrX: 66795770- | TTTCACATGAAAAAGACAAATGAGGCAAAGGAAGAGAAAAATGCATTGTCAGAATCAGAATTATGAGAAAAAAAGTCAAACAAACATATT | 397 |
| 66944119 | 66795889 | TGAAATGTCCAGAAAACCTGTGAGTTTTTA | |
| chrX: 66763874- | chrX: 66795470- | ATACTGAGAAGGGCTGATGGCTGAAGGAGGAACAATTTAAAAGAATAACCGTCTCTCCTCTCCCTGTATATTGGACATAAAAGAATATCC | 398 |
| 66944119 | 66795589 | CATTCTTTTCAGAAATGTAATACAACAGTT | |
| chrX: 66763874- | chrX: 66795530- | TCCCTGTATATTGGACATAAAAGAATATCCCATTCTTTTCAGAAATGTAATACAACAGTTTAGCTTGCTAGTAACTTCACATGCTATTTC | 399 |
| 66944119 | 66795649 | CTTTACCTCTTATATTTGAGGTGTCTATTT | |
| chrX: 66763874- | chrX: 66795050- | AAGGAGAGGGAGAGAGAAAAGGAAAAACTGGGCCACCCATCCTTTGAAAAGAAACCTTGAAAGAGGTCCAAATATCCTTAGAAATCCTTG | 400 |
| 66944119 | 66795169 | ACTTCTTAAAAGTGATGTTTGTTTTTTCCC | |
| chrX: 66763874- | chrX: 66794390- | TCTGACTTTATGGCCTCTCAGCTTTCAATGACTAGCTTTGTAGCAGAAGTTTAGCCTCTCATCCCCATAACTTTGGAAGTAGTGTTGAGA | 401 |
| 66944119 | 66794509 | TAAAGAAACGTTGAATTGAAGGTTGTGTTT | |
| chrX: 66763874- | chrX: 66795890- | TGTATACTATACAGGAAAGATATTCTGTCATCTGGTTGCCAAACTATGGAGGGTGGGAGACTTCGAATTTTTGTCAAAAAGTATTCTTTC | 402 |
| 66944119 | 66796009 | ATTAGAAAGATACATGGGTGTGCTTCCATG | |
| chrX: 66763874- | chrX: 66796430- | CTCCTCCTAATACACTTATCCACGTTTGGATACCTTGGTCTCAGCCTCAGAGGTCATATTTTTAGTAAAATGGCCACCAGAAATAAAGGA | 403 |
| 66944119 | 66796549 | TTTTATTTTCCAGACTTTGGTGTTTGGAGC | |
| chrX: 66763874- | chrX: 66795710- | CTGGTATAAATTTTATTTGGGTAAATATCACCTCAATTTTCAACTAAAGCTTTATTTAAGTTTCACATGAAAAAGACAAATGAGGCAAAG | 404 |
| 66944119 | 66795829 | GAAGAGAAAAATGCATTGTCAGAATCAGAA | |
| chrX: 66763874- | chrX: 66793970- | TTCTTGCAAGTGGTGGTTTACTTCACTTCAACTATAGAAGGCCTATGCGACACCACCCATAGAGGGTAGTTTGAAAGAAAATGCTAGTGA | 405 |
| 66944119 | 66794089 | CTACGTGTGTTTCCTTCCTGACATATTTTA | |
| chrX: 66763874- | chrX: 66796010- | TCAGCAACATGACTGCAGACCAGGAAGTCCTCACGGAGAGCTGGAATATGGGTATTTTGGACTCTCTGGTTAGATGCAGCTTTTACTTCA | 406 |
| 66944119 | 66796129 | CATCCTCAGTGGTACTACTGTAAATTTTCA | |
| chrX: 66763874- | chrX: 66793910- | TGTACGGAAAATAGGAGTTGATAATTTTTAAGGCCCTTGCCAGCACATTAGTACATAGGATTCTTGCAAGTGGTGGTTTACTTCACTTCA | 407 |
| 66944119 | 66794029 | ACTATAGAAGGCCTATGCGACACCACCCAT | |
| chrX: 66763874- | chrX: 66795110- | AAGAGGTCCAAATATCCTTAGAAATCCTTGACTTCTTAAAAGTGATGTTTGTTTTTTCCCCCTGACAATTATAGAGGTCAGAGAGTTTTT | 408 |
| 66944119 | 66795229 | CTTTTCTATTACAAAACATTGAGAGTGTGT | |
| chrX: 66763874- | chrX: 66796370- | ATGAAAAATTTATTTGAAAATAACAGCACAGAAAGGAAGAAAGACAGGCTGGCAAGCATCCTCCTCCTAATACACTTATCCACGTTTGGA | 409 |
| 66944119 | 66796489 | TACCTTGGTCTCAGCCTCAGAGGTCATATT | |
| chrX: 66763874- | chrX: 66795170- | CCTGACAATTATAGAGGTCAGAGAGTTTTTCTTTTCTATTACAAAACATTGAGAGTGTGTAGAAATAATTGTAGGTAGCTTAGCCTTGGC | 410 |
| 66944119 | 66795289 | TGTAGTCAGAACTTTTGTACTGTGACTTTA | |
| chrX: 66763874- | chrX: 66793850- | AGACAAAATTCTTTCCATTCTCCAGCTTATATTTTCCCCATTTGTAAAACATAATGGAAGTGTACGGAAAATAGGAGTTGATAATTTTTA | 411 |
| 66944119 | 66793969 | AGGCCCTTGCCAGCACATTAGTACATAGGA | |
| chrX: 66763874- | chrX: 66794510- | TCTAGATTTCTTTCAATTGCTCCTTAGGCTTTAGAAGATAAATTCTCCTAAAAGAGAGGTGCTACAATTAATCCAAGCAAAGGGAAAGAT | 412 |
| 66944119 | 66794629 | GTCAGTAAAACTGCCCCTTTTCATAGAGGT | |
| chrX: 66763874- | chrX: 66796130- | TTTTCCTGTGGAATACCCTATTTGGTTCCATTGTATATAGTTGACAACTAGAATTCGTTCGCTGTTGCTTGAGCCCAACTATAACTTCTT | 413 |
| 66944119 | 66796249 | GGCACTATACCTATCTTCTGATGTGCCTGT | |
| chrX: 66763874- | chrX: 66794270- | GACCCATTGGGCTAGTTTGAACGTGTGCAGTCTCTGTGCTCCCCGTTTTAGCTTAAGCCTACTCCCTAACCTGTCATATGTCACCCAGCC | 414 |
| 66944119 | 66794389 | ATGGAGCCTAGGGCAATGACTGCCATCATA | |
| chrX: 66763874- | chrX: 66795410- | AGATGCTTAAAGGAAGGCAGTGCAGACTTTCAGAAACTAGACTTTAAGAGCTGTACTCAGATACTGAGAAGGGCTGATGGCTGAAGGAGG | 415 |
| 66944119 | 66795529 | AACAATTTAAAAGAATAACCGTCTCTCCTC | |
| chrX: 66763874- | chrX: 66794330- | ACTCCCTAACCTGTCATATGTCACCCAGCCATGGAGCCTAGGGCAATGACTGCCATCATATCTGACTTTATGGCCTCTCAGCTTTCAATG | 416 |
| 66944119 | 66794449 | ACTAGCTTTGTAGCAGAAGTTTAGCCTCTC | |
| chrX: 66763874- | chrX: 66796310- | TAAATCATGAGTTTGTGCCTTGGGAGCTACAGTTTAAACATTTGCTGTTTTTCTCACTTAATGAAAAATTTATTTGAAAATAACAGCACA | 417 |
| 66944119 | 66796429 | GAAAGGAAGAAAGACAGGCTGGCAAGCATC | |
| chrX: 66763874- | chrX: 66794450- | ATCCCCATAACTTTGGAAGTAGTGTTGAGATAAAGAAACGTTGAATTGAAGGTTGTGTTTTCTAGATTTCTTTCAATTGCTCCTTAGGCT | 418 |
| 66944119 | 66794569 | TTAGAAGATAAATTCTCCTAAAAGAGAGGT | |
| chrX: 66763874- | chrX: 66794750- | CTGGCAGCACCAAACAGTAAGGAAACCACAAAGATAAACCCAAATAATAGAGCCAATTTTCTTTTTTTCCGGGGGGGATGACTTCTAACT | 419 |
| 66944119 | 66794869 | AGTGATATGAGGAAGGATAAGAAAATGTTT | |
| chrX: 66763874- | chrX: 66795290- | GGATCTGTATGGAATCGTATGATATGCGGATACACCAAAAACTCTATGGGTTATCAAAATGGGATAGCATTAAAAGAAATAGTGCTTTTG | 420 |
| 66944119 | 66795409 | TTTAGAAGAAGAAATGAAATGCTTGTGTCC | |
| chrX: 66763874- | chrX: 66798216- | GTCTCTATGTACTTGTGTGTGAAACAATGAGCACAAATAATACCCTCCTTGTTTTTAAGCAATTTATATTGGTGATTTAAAAATAAAATA | 421 |
| 66944119 | 66798335 | AACTCAAGTGGGAAATCATGAAACCCCATG | |
| chrX: 66763874- | chrX: 66797856- | ACTGAGAATGCAACCCAAGAACAGAAATTTGTCAGAAATTTAGCACTGAAGCCCCCCACTTCCCAAACTTATCTGGGACAAGGAGAATCT | 422 |
| 66944119 | 66797975 | ACATTTAAAGCTCTATACTTTGTGTTGTGT | |
| chrX: 66763874- | chrX: 66797796- | TGCATTAGCCACTTTGCTGGGTATGAATGCCAGCAGAATCTAAGTGACCTTGGCTTCACTACTGAGAATGCAACCCAAGAACAGAAATTT | 423 |
| 66944119 | 66797915 | GTCAGAAATTTAGCACTGAAGCCCCCCACT | |
| chrX: 66763874- | chrX: 66798036- | CCTAAGCAGAGGCAAGGGAGGAAAGGGATATGAACCTGGTAGAAAAGTAAGTAAGCTTTATTCAGATTGGCATATCCATCTTAATATGGT | 424 |
| 66944119 | 66798155 | TCAATTGGCTGAAGAAGTATCTCAACTAAA | |
| chrX: 66763874- | chrX: 66797916- | TCCCAAACTTATCTGGGACAAGGAGAATCTACATTTAAAGCTCTATACTTTGTGTTGTGTTTTTTTTACTTTAGCTTGGTTGGATTTAGG | 425 |
| 66944119 | 66798035 | ATCTTTTCTTTTTGTTTTGCCTTATGCATA | |
| chrX: 66763874- | chrX: 66797976- | TTTTTTTACTTTAGCTTGGTTGGATTTAGGATCTTTTCTTTTTGTTTTGCCTTATGCATACCTAAGCAGAGGCAAGGGAGGAAAGGGATA | 426 |
| 66944119 | 66798095 | TGAACCTGGTAGAAAAGTAAGTAAGCTTTA | |
| chrX: 66763874- | chrX: 66797736- | AGTTATATGATGTTTTCTAGCAAGCATTTGCGTTGTTCTACTGGTGTTACATATCTTAGCTGCATTAGCCACTTTGCTGGGTATGAATGC | 427 |
| 66944119 | 66797855 | CAGCAGAATCTAAGTGACCTTGGCTTCACT | |
| chrX: 66763874- | chrX: 66798096- | TTCAGATTGGCATATCCATCTTAATATGGTTCAATTGGCTGAAGAAGTATCTCAACTAAAACTCTGGAATACTTTGAAGTACCAGCAATA | 428 |
| 66944119 | 66798215 | TGTACCAAATGTACTTTTTATTTATGTTTG | |
| chrX: 66763874- | chrX: 66798156- | ACTCTGGAATACTTTGAAGTACCAGCAATATGTACCAAATGTACTTTTTATTTATGTTTGGTCTCTATGTACTTGTGTGTGAAACAATGA | 429 |
| 66944119 | 66798275 | GCACAAATAATACCCTCCTTGTTTTTAAGC | |
| chrX: 66763874- | chrX: 66798655- | ACTTTGAACATTTATATTCTGCTTTATTTAGGCATAAGTGCTTAATAATTATTGATAGTTTCTTCTGGTTATCTGACATTTTGAAGATAC | 430 |
| 66944119 | 66798774 | TATTACCTAGCAGAAATTTCTTGtaataat | |
| chrX: 66763874- | chrX: 66798595- | TTGCAGTCAAATCTGGCATGAAATTAGTGCATAGACAGAATGGGCTGGGAAAATGAAAGGACTTTGAACATTTATATTCTGCTTTATTTA | 431 |
| 66944119 | 66798714 | GGCATAAGTGCTTAATAATTATTGATAGTT | |
| chrX: 66763874- | chrX: 66798535- | agtgttcaTATTTTGTATTTGAGCACAGGGCAACTGGGTTTTTGAAACTGCACATTACTGTTGCAGTCAAATCTGGCATGAAATTAGTGC | 432 |
| 66944119 | 66798654 | ATAGACAGAATGGGCTGGGAAAATGAAAGG | |
| chrX: 66763874- | chrX: 66799958- | AGTTCATCTTAAGCATATGGCTGTCTGTCTTTTCTCTAAAGATCCTCAAGGGAAAAAAAAAAAAGCATCTCCAGGGGGAATTTACTGCCT | 433 |
| 66944119 | 66800077 | CATAGCCCTGACAGAGATTTCTGACCAAAC | |
| chrX: 66763874- | chrX: 66799058- | CTTGAATATCCTTTTAGCGACTTGCCTTTGGTGTTAGTGTGCCTATAACATTGTCGTTGAATATCTTAATACATTTAGTGGTCTTGGCAA | 434 |
| 66944119 | 66799177 | GCAGTTTTGTCTTCAGAAGGACACTGAAAT | |
| chrX: 66763874- | chrX: 66799118- | ATATCTTAATACATTTAGTGGTCTTGGCAAGCAGTTTTGTCTTCAGAAGGACACTGAAATCTGTGGAAAGGACTGCAGAAGATTGGGTGG | 435 |
| 66944119 | 66799237 | GCAGACACCTATCACTTTCGGGGCTGGTAG | |
| chrX: 66763874- | chrX: 66799238- | ACTTTCTATTGAAGCAATTTGCAAGGCTACTTTGTATTGTCTAAAAGCACTACTTCAGAAAAGGGTTGTGATGTCAAAATAGGCACTTTG | 436 |
| 66944119 | 66799357 | AGTGAAGAAAGGGCTGTAAGCATGGGTGGA | |
| chrX: 66763874- | chrX: 66800198- | AAAATATACACAGCTTTGGATTTCCTTATTATGGCCCTTCATTAAGTTGTGGTTTAAGAATAGCTATGATTATTACTTTTGTGATAATTA | 437 |
| 66944119 | 66800317 | TAATCCATAATATGGAAACTTATAAAATTA | |
| chrX: 66763874- | chrX: 66799358- | AAATGTGGTAGATGATTGTCTTGAGTTATTTTCTTTAATGTCAAACAGGCAGTCCTTGGAATGCTACTTCAAAAAGTGTTGTATAATGTT | 438 |
| 66944119 | 66799477 | GAAGATACAGTTACAGATTTCCAACACGAA | |
| chrX: 66763874- | chrX: 66800078- | CCTAACGAAAAAATTTCTTCCCTCCATTTGTCTTTTATTGTTTTTACAGGGGAGATATGTAACATAATAACAATTATATTGCACATAATA | 439 |
| 66944119 | 66800197 | ATTACTTCTACAAATAATAATCTGTTGTCA | |
| chrX: 66763874- | chrX: 66800138- | AACATAATAACAATTATATTGCACATAATAATTACTTCTACAAATAATAATCTGTTGTCAAAAATATACACAGCTTTGGATTTCCTTATT | 440 |
| 66944119 | 66800257 | ATGGCCCTTCATTAAGTTGTGGTTTAAGAA | |
| chrX: 66763874- | chrX: 66799778- | TCTCTGAGTAGTACCATAAGTTGTTAGTCTGCTACTCTTTCTCCCAGTTGGCACATGACCCTAACATCCAATCGCTAGTGGTGTGGCCAT | 441 |
| 66944119 | 66799897 | TTTTTGGTCTTATTTTGGCCTTTCCTCAGC | |
| chrX: 66763874- | chrX: 66799718- | CAGCCTTTTTGCACAACAAAATGGCAGCACCCAGGAGGTTGAAAGGGTTAAATTGTTCCTTCTCTGAGTAGTACCATAAGTTGTTAGTCT | 442 |
| 66944119 | 66799837 | GCTACTCTTTTTCCCAGTTGGCACATGACC | |
| chrX: 66763874- | chrX: 66800258- | TAGCTATGATTATTACTTTTGTGATAATTATAATCCATAATATGGAAACTTATAAAATTACCTTTAAAGTGTTACTATTATTCTGGCCAC | 443 |
| 66944119 | 66800377 | AGGATGGAAAGTTGTTCGCTAGTTACTCAT | |
| chrX: 66763874- | chrX: 66799418- | ATGCTACTTCAAAAAGTGTTGTATAATGTTGAAGATACAGTTACAGATTTCCAACACGAAACTCATAAATATGCAATTCCCTGTCCTCCT | 444 |
| 66944119 | 66799537 | AGGCACATGAAGGAAAATTTATGAGCTTCA | |
| chrX: 66763874- | chrX: 66799538- | GGTTTCTATGCAGCTATTAAAGCATATTTAATCTGCTTTGAGCTCAAGCTCACTCTCGTTGGCTCTCTTCGTTTCTTCCTCTTACATGAG | 445 |
| 66944119 | 66799657 | CAAACTGCCTTTCTTTTTGTTTAAAAATAG | |
| chrX: 66763874- | chrX: 66799298- | AAGGGTTGTGATGTCAAAATAGGCACTTTGAGTGAAGAAAGGGCTGTAAGCATGGGTGGAAAATGTGGTAGATGATTGTCTTGAGTTATT | 446 |
| 66944119 | 66799417 | TTCTTTAATGTCAAACAGGCAGTCCTTGGA | |
| chrX: 66763874- | chrX: 66799478- | ACTCATAAATATGCAATTCCCTGTCCTCCTAGGCACATGAAGGAAAATTTATGAGCTTCAGGTTTCTATGCAGCTATTAAAGCATATTTA | 447 |
| 66944119 | 66799597 | ATCTGCTTTGAGCTCAAGCTCACTCTCGTT | |
| chrX: 66763874- | chrX: 66800318- | CCTTTAAAGTGTTACTATTATTCTGGCCACAGGATGGAAAGTTGTTCGCTAGTTACTCATTTATAACCTGAATGTACTTTTTACTGAATC | 448 |
| 66944119 | 66800437 | TAAAGGTATCAtctttgcttggcaattccc | |
| chrX: 66763874- | chrX: 66799838- | CTAACATCCAATCGCTAGTGGTGTGGCCATTTTTTGGTCTTATTTTGGCCTTTCCTCAGCCACCACTCATCAGTTCTCATGCGTATTTGT | 449 |
| 66944119 | 66799957 | CAGATCCTGCTCCCCAACTCCACAGTTCTT | |
| chrX: 66763874- | chrX: 66799658- | TAAGTAGGTTTGTTTTCCTCCAGGTGTCATGAATGCAAACATTGTAATTTCTCATCTGTTCAGCCTTTTTGCACAACAAAATGGCAGCAC | 450 |
| 66944119 | 66799777 | CCAGGAGGTTGAAAGGGTTAAATTGTTCCT | |
| chrX: 66763874- | chrX: 66799898- | CACCACTCATCAGTTCTCATGCGTATTTGTCAGATCCTGCTCCCCAACTCCACAGTTCTTAGTTCATCTTAAGCATATGGCTGTCTGTCT | 451 |
| 66944119 | 66800017 | TTTCTCTAAAGATCCTCAAGGGAAAAAAAA | |
| chrX: 66763874- | chrX: 66800018- | AAAAGCATCTCCAGGGGGAATTTACTGCCTCATAGCCCTGACAGAGATTTCTGACCAAACCCTAACGAAAAAATTTCTTCCCTCCATTTG | 452 |
| 66944119 | 66800137 | TCTTTTATTGTTTTTACAGGGGAGATATGT | |
| chrX: 66763874- | chrX: 66799178- | CTGTGGAAAGGACTGCAGAAGATTGGGTGGGCAGACACCTATCACTTTCGGGGCTGGTAGACTTTCTATTGAAGCAATTTGCAAGGCTAC | 453 |
| 66944119 | 66799297 | TTTGTATTGTCTAAAAGCACTACTTCAGAA | |
| chrX: 66763874- | chrX: 66799598- | GGCTCTCTTCGTTTCTTCCTCTTACATGAGCAAACTGCCTTTCTTTTTGTTTAAAAATAGTAAGTAGGTTTGTTTTCCTCCAGGTGTCAT | 454 |
| 66944119 | 66799717 | GAATGCAAACATTGTAATTTCTCATCTGTT | |
| chrX: 66763874- | chrX: 66801296- | TATCATGTTCAGGCCTTCTTTCCAGCATGTGGCTCTCAGCCCTGGTACTGTCCTTAACCATAAACCTCATCTTTGCCCTCTATAGGGAGA | 455 |
| 66944119 | 66801415 | GGTTTATGGTTATAATTACTCATTTTAAAT | |
| chrX: 66763874- | chrX: 66801116- | CTTCAAGAAGTTTTTACGTACCTCTTATATAGAATGTGATGTTTTATATGTACCTCTTATAGAATGTGAGCTTTTAAGAGGCATATCTTA | 456 |
| 66944119 | 66801235 | TTGCAAGAAATTTCAATGTTGAAAAAAATA | |
| chrX: 66763874- | chrX: 66801056- | ACAAATCTTTAATTCACATGGCAATTTCTAGGATTTATCATGGAAAATGAGCCAAATTGCCTTCAAGAAGTTTTTACGTACCTCTTATAT | 457 |
| 66944119 | 66801175 | AGAATGTGATGTTTTATATGTACCTCTTAT | |
| chrX: 66763874- | chrX: 66801236- | TTGAATATTTATAAAGTCAAAAATGCAAACTTTTATATGATTTTCAAACCTATGAAGTTATATCATGTTCAGGCCTTCTTTCCAGCATGT | 458 |
| 66944119 | 66801355 | GGCTCTCAGCCCTGGTACTGTCCTTAACCA | |
| chrX: 66763874- | chrX: 66801176- | AGAATGTGAGCTTTTAAGAGGCATATCTTATTGCAAGAAATTTCAATGTTGAAAAAAATATTGAATATTTATAAAGTCAAAAATGCAAAC | 459 |
| 66944119 | 66801295 | TTTTATATGATTTTCAAACCTATGAAGTTA | |
| chrX: 66763874- | chrX: 66802336- | GAGTCAACTGTAAGTAGACAGAATTGCCTTTGACTTAATCTGTTTCAGTCGTTGTTCATACTCAGGTCCTCCAGAGGACCTTTAAGCATT | 460 |
| 66944119 | 66802455 | TTTATTGACTTTGTGGTCTATTACACGAAA | |
| chrX: 66763874- | chrX: 66802156- | GAGCACATAACATTCTTTTGTGCTAACAGTATCTCTGCATCACATTGATCAGGAGAATTGGCATCTCCAGAGCCCTGGGATGGTAACTTC | 461 |
| 66944119 | 66802275 | TCTGTTGATTTTCAGGAAAGATTAGGTGAT | |
| chrX: 66763874- | chrX: 66801676- | AGAAATTATTAGTTAAAGAAATAATAGAATTTTACAAGACTCTAGGAAGGGAGAATGTGAAGGATACAGTTCTCAGTTACTGGAATGAGT | 462 |
| 66944119 | 66801795 | GCCAGAGTACCAGTACATGGCTTGCCTTGG | |
| chrX: 66763874- | chrX: 66801916- | TCAGATTCAGTTAAAGTTTACTTTCCTGACAGCTTTTTAGTATCATATCTATTTTGCAAAACTCTAGTGATAAATGTATGCACATTTACA | 463 |
| 66944119 | 66802035 | CATACAGCATCTCTTCTGATTCTGACTAAG | |
| chrX: 66763874- | chrX: 66802216- | GCATCTCCAGAGCCCTGGGATGGTAACTTCTCTGTTGATTTTCAGGAAAGATTAGGTGATATTTTCTCCATGGGAAGAGGATGTTTGATG | 464 |
| 66944119 | 66802335 | TGTGTTGGCTTTAGCAAAAGGAAGCTTGTG | |
| chrX: 66763874- | chrX: 66801796- | GGTTTGGACTACCTATCTTAACTCCTTTGCTCCTCCCAATCTTGATCTCATTTGTTTGAAAGATCATCTGCCCAACATAAAAATGCATTT | 465 |
| 66944119 | 66801915 | CTAATTCTGTAATTTAAGTCAGTGGCAAGA | |
| chrX: 66763874- | chrX: 66802096- | ATCTAAGGACTGAATTTTCTCATCTTTGTCTTTGCCCCTTTTGACTGATGACCAGAGCAGGAGCACATAACATTCTTTTGTGCTAACAGT | 466 |
| 66944119 | 66802215 | ATCTCTGCATCACATTGATCAGGAGAATTG | |
| chrX: 66763874- | chrX: 66802276- | ATTTTCTCCATGGGAAGAGGATGTTTGATGTGTGTTGGCTTTAGCAAAAGGAAGCTTGTGGAGTCAACTGTAAGTAGACAGAATTGCCTT | 467 |
| 66944119 | 66802395 | TGACTTAATCTGTTTCAGTCGTTGTTCATA | |
| chrX: 66763874- | chrX: 66801736- | AGGATACAGTTCTCAGTTACTGGAATGAGTGCCAGAGTACCAGTACATGGCTTGCCTTGGGGTTTGGACTACCTATCTTAACTCCTTTGC | 468 |
| 66944119 | 66801855 | TCCTCCCAATCTTGATCTCATTTGTTTGAA | |
| chrX: 66763874- | chrX: 66802036- | ATATTACTGGGTTGTGTAGAAGTGATGGGCTCTTTAGAAGAAAGGTTTGATATACTACTAATCTAAGGACTGAATTTTCTCATCTTTGTC | 469 |
| 66944119 | 66802155 | TTTGCCCCTTTTGACTGATGACCAGAGCAG | |
| chrX: 66763874- | chrX: 66801616- | GTGAGAAAAAAGCAGCATCCATAAGGTTTTCATTCTCCTACCCTGTACGACAGAGGTAATAGAAATTATTAGTTAAAGAAATAATAGAAT | 470 |
| 66944119 | 66801735 | TTTACAAGACTCTAGGAAGGGAGAATGTGA | |
| chrX: 66763874- | chrX: 66801856- | AGATCATCTGCCCAACATAAAAATGCATTTCTAATTCTGTAATTTAAGTCAGTGGCAAGATCAGATTCAGTTAAAGTTTACTTTCCTGAC | 471 |
| 66944119 | 66801975 | AGCTTTTTAGTATCATATCTATTTTGCAAA | |
| chrX: 66763874- | chrX: 66801976- | ACTCTAGTGATAAATGTATGCACATTTACACATACAGCATCTCTTCTGATTCTGACTAAGATATTACTGGGTTGTGTAGAAGTGATGGGC | 472 |
| 66944119 | 66802095 | TCTTTAGAAGAAAGGTTTGATATACTACTA | |
| chrX: 66763874- | chrX: 66803114- | AAAAGAACCACATTCTATTCACATGCTTCATTTTATTCTGATTTATGTAAAAATTCCCAAACTCCTCAAGCAGTGTTTCTTTGTAAGGCA | 473 |
| 66944119 | 66803233 | ATAATCTTCAGTTCTGTTGCAAAGGTCAGG | |
| chrX: 66763874- | chrX: 66802694- | TGGGCCTAGAGGGTTATATAAGATTTCAATATTAAAACATGGATTAAAAGTGAAGACTTTTCACATGGAGATAATTTGGAAGAAAAACTT | 474 |
| 66944119 | 66802813 | GCAAAAATGTGAGAGCATTGAGAACTTTTC | |
| chrX: 66763874- | chrX: 66802994- | CTACTATGAAATCTCTGTTCCCAGCTAGAGGCCTGGGAGAGTAAGATAACTACTTGTTTATTCCACGGAGCCACTTATTAGCTTTTTCTA | 475 |
| 66944119 | 66803113 | TAGCACATACCTCAAATGAAGCATTTCAAT | |
| chrX: 66763874- | chrX: 66803174- | ACTCCTCAAGCAGTGTTTCTTTGTAAGGCAATAATCTTCAGTTCTGTTGCAAAGGTCAGGAGTGATAGAATGAAAATGGTACTAGATACA | 476 |
| 66944119 | 66803293 | ACAGCTCTTTGGTATTTGCATGGCCATTAC | |
| chrX: 66763874- | chrX: 66802814- | TTTCCCAAGGAAAGAAGTGGCAGCTTCATTTTTGGTCATTGCAAACAGCAGTGCCATACATGAAAGGAAAGTGGTGGTGCTCATCAACTT | 477 |
| 66944119 | 66802933 | TGAATAACTTTGTACAGAACCCTTGAGACT | |
| chrX: 66763874- | chrX: 66803954- | TTTGTTACTACTTATTGGGGATCCTGGAAAGAAAATATATTGTCTATATCCACTGTTCACTGAGGCCCTCTCCCTACCCAGAAACTCCCT | 478 |
| 66944119 | 66804073 | GTCTCCATCACTCACTCTCCACATTCATTG | |
| chrX: 66763874- | chrX: 66802754- | TCACATGGAGATAATTTGGAAGAAAAACTTGCAAAAATGTGAGAGCATTGAGAACTTTTCTTTCCCAAGGAAAGAAGTGGCAGCTTCATT | 479 |
| 66944119 | 66802873 | TTTGGTCATTGCAAACAGCAGTGCCATACA | |
| chrX: 66763874- | chrX: 66803894- | AGGTAGTTCTCAAATATTTACTCCATGAATAGTTGCTGGATGTTCATTAACTCTATAGCATTTGTTACTACTTATTGGGGATCCTGGAAA | 480 |
| 66944119 | 66804013 | GAAAATATATTGTCTATATCCACTGTTCAC | |
| chrX: 66763874- | chrX: 66804134- | ATTTCAAGGCAAGAGGTATAAGAGAAAGTTCAGAGACAACACTGGCTATGGTCTTTGTGAAGAAAAGTGAATTGAATAGGCTCCTGTGGA | 481 |
| 66944119 | 66804253 | GATCTTAAGTAAGTACTTCTGGAGATAAGG | |
| chrX: 66763874- | chrX: 66802934- | CCTCTCTGCTTATAAAGAAAAAGTGTCAACTGTAAAGTTGATTTATTTATGAACCATAGGCTACTATGAAATCTCTGTTCCCAGCTAGAG | 482 |
| 66944119 | 66803053 | GCCTGGGAGAGTAAGATAACTACTTGTTTA | |
| chrX: 66763874- | chrX: 66803714- | CTGACCCCTAGAAAATCCTGGTGAAGTTTTTCTGGTGTCAGTTTGGTCTTAATGTTTAGGAAATGCCCACAGACTACTCCTGCTTTCTGC | 483 |
| 66944119 | 66803833 | TTATTCACATAGTAAACGCAAAGCACAGGA | |
| chrX: 66763874- | chrX: 66804194- | AGAAAAGTGAATTGAATAGGCTCCTGTGGAGATCTTAAGTAAGTACTTCTGGAGATAAGGTTGAGGAAAAGTAGGTTTGAATCTTCATCC | 484 |
| 66944119 | 66804313 | AGAGGTAGCCCCTAAATGTGTTGAGTTTAT | |
| chrX: 66763874- | chrX: 66802874- | TGAAAGGAAAGTGGTGGTGCTCATCAACTTTGAATAACTTTGTACAGAACCCTTGAGACTCCTCTCTGCTTATAAAGAAAAAGTGTCAAC | 485 |
| 66944119 | 66802993 | TGTAAAGTTGATTTATTTATGAACCATAGG | |
| chrX: 66763874- | chrX: 66804074- | ACCCAGGGGAACAGTTCATGGATGAGTGAACTTGAGCTCTATCTTAAAGGATGGAGTTCGATTTCAAGGCAAGAGGTATAAGAGAAAGTT | 486 |
| 66944119 | 66804193 | CAGAGACAACACTGGCTATGGTCTTTGTGA | |
| chrX: 66763874- | chrX: 66803534- | TATTCAACAACCATAAAAAAAAACAATTAGGACTCAAGTAGTATGTCAGAGTGTAGTCACTGATGATATATAATTCTCCACTACCAAGAA | 487 |
| 66944119 | 66803653 | GATGGAAGCACACTGTTGAGTAGCTACATC | |
| chrX: 66763874- | chrX: 66803654- | CTACATATGTTGGCCAGAATTTAGGAATACACATGTGATCTATACATTTTGAGGTATTGTCTGACCCCTAGAAAATCCTGGTGAAGTTTT | 488 |
| 66944119 | 66803773 | TCTGGTGTCAGTTTGGTCTTAATGTTTAGG | |
| chrX: 66763874- | chrX: 66803054- | TTCCACGGAGCCACTTATTAGCTTTTTCTATAGCACATACCTCAAATGAAGCATTTCAATAAAAGAACCACATTCTATTCACATGCTTCA | 489 |
| 66944119 | 66803173 | TTTTATTCTGATTTATGTAAAAATTCCCAA | |
| chrX: 66763874- | chrX: 66803354- | TGTGTTTGTGCTAATGGAGTGATTTGAGAGGTAGTTCTCCACTGTCAGTCAAGAGGTTGGTTTTGAAAGCTGATTGCCAATGGTCATTCT | 490 |
| 66944119 | 66803473 | GCTAACCACTCTGGTTCTCCTTTAGATAGA | |
| chrX: 66763874- | chrX: 66803594- | TGATGATATATAATTCTCCACTACCAAGAAGATGGAAGCACACTGTTGAGTAGCTACATCCTACATATGTTGGCCAGAATTTAGGAATAC | 491 |
| 66944119 | 66803713 | ACATGTGATCTATACATTTTGAGGTATTGT | |
| chrX: 66763874- | chrX: 66803474- | GACTTATTCAGATTCAAGTCTTCATGTACTTTGTGGCATAAACATTGTACACACCAGATGTATTCAACAACCATAAAAAAAAACAATTAG | 492 |
| 66944119 | 66803593 | GACTCAAGTAGTATGTCAGAGTGTAGTCAC | |
| chrX: 66763874- | chrX: 66803774- | AAATGCCCACAGACTACTCCTGCTTTCTGCTTATTCACATAGTAAACGCAAAGCACAGGACTAGTTTGTCATCTGGATCAAGGAGAAATG | 493 |
| 66944119 | 66803893 | AGTTAGCAGATATAAAATAAATCAGAAAGG | |
| chrX: 66763874- | chrX: 66802634- | GTGGCTGAACTTCTCCACTCCCGTATCCATCGCAATACTTCCCCAAGGTGGCATTTAAGATGGGCCTAGAGGGTTATATAAGATTTCAAT | 494 |
| 66944119 | 66802753 | ATTAAAACATGGATTAAAAGTGAAGACTTT | |
| chrX: 66763874- | chrX: 66803294- | ATTGCCATGGGGCTGCAAGACTTGTGAGTGCTTGATATTTTGCTTGTTGATGAATGAGTCTGTGTTTGTGCTAATGGAGTGATTTGAGAG | 495 |
| 66944119 | 66803413 | GTAGTTCTCCACTGTCAGTCAAGAGGTTGG | |
| chrX: 66763874- | chrX: 66803414- | TTTTGAAAGCTGATTGCCAATGGTCATTCTGCTAACCACTCTGGTTCTCCTTTAGATAGAGACTTATTCAGATTCAAGTCTTCATGTACT | 496 |
| 66944119 | 66803533 | TTGTGGCATAAACATTGTACACACCAGATG | |
| chrX: 66763874- | chrX: 66803234- | AGTGATAGAATGAAAATGGTACTAGATACAACAGCTCTTTGGTATTTGCATGGCCATTACATTGCCATGGGGCTGCAAGACTTGTGAGTG | 497 |
| 66944119 | 66803353 | CTTGATATTTTGCTTGTTGATGAATGAGTC | |
| chrX: 66763874- | chrX: 66804014- | TGAGGCCCTCTCCCTACCCAGAAACTCCCTGTCTCCATCACTCACTCTCCACATTCATTGACCCAGGGGAACAGTTCATGGATGAGTGAA | 498 |
| 66944119 | 66804133 | CTTGAGCTCTATCTTAAAGGATGGAGTTCG | |
| chrX: 66763874- | chrX: 66803834- | CTAGTTTGTCATCTGGATCAAGGAGAAATGAGTTAGCAGATATAAAATAAATCAGAAAGGAGGTAGTTCTCAAATATTTACTCCATGAAT | 499 |
| 66944119 | 66803953 | AGTTGCTGGATGTTCATTAACTCTATAGCA | |
| chrX: 66763874- | chrX: 66804597- | TGTCTTCCAACTCTCCCAGGCTTCTTTGTTTGCAAGGCTGACTTTTATAATACTTTTTGGGTAGAGCAGGTCCTTCTTTGGTTTGGGGTT | 500 |
| 66944119 | 66804716 | AAACCGTGAGTAACCTTATTTTCTAGGTCT | |
| chrX: 66763874- | chrX: 66804417- | GCCAAGCACTCAGTAAATGTTTGAATGGGAAAATTAACTGCCCTGTTTTTCTATTGTCAGATGGTCCTCTTCGTTGGATAACTTGGTAAC | 501 |
| 66944119 | 66804536 | TGTTGATAACCTTTTCTCAGGAATCAGAAG | |
| chrX: 66763874- | chrX: 66804537- | GTAGAAAGGTTGGGAAAATATAAGAAACAAAAAGGCATATTCCTATTTTTATTTTCATATTGTCTTCCAACTCTCCCAGGCTTCTTTGTT | 502 |
| 66944119 | 66804656 | TGCAAGGCTGACTTTTATAATACTTTTTGG | |
| chrX: 66763874- | chrX: 66804657- | GTAGAGCAGGTCCTTCTTTGGTTTGGGGTTAAACCGTGAGTAACCTTATTTTCTAGGTCTCAGCCAACTTTGAAGGGCATGAACTCACAG | 503 |
| 66944119 | 66804776 | TAGCCTCACTAGGATCACTTCAGCAGTGAG | |
| chrX: 66763874- | chrX: 66804477- | ATGGTCCTCTTCGTTGGATAACTTGGTAACTGTTGATAACCTTTTCTCAGGAATCAGAAGGTAGAAAGGTTGGGAAAATATAAGAAACAA | 504 |
| 66944119 | 66804596 | AAAGGCATATTCCTATTTTTATTTTCATAT | |
| chrX: 66763874- | chrX: 66805205- | AATATGGACTGAGTTTCTGTGGGGTGGAAATGTGAAGTGGATCATAGCATGATATAACTTGTCATTTGGCTTCCTTTATAAACATTATCA | 505 |
| 66944119 | 66805324 | ACTACCTCAGCTCTATCAATCACTTGGCAG | |
| chrX: 66763874- | chrX: 66805265- | GTCATTTGGCTTCCTTTATAAACATTATCAACTACCTCAGCTCTATCAATCACTTGGCAGTCCGTAGTGAACATTATAACTCAAATGACT | 506 |
| 66944119 | 66805384 | AGTCAGGTCTGTTCATTGCCCATGTAAAGG | |
| chrX: 66763874- | chrX: 66805505- | CTTTGTCACAAGTAAGAAGGTTTAAAAATCACCATACCATTATTGGTCACAACGTTTGGAGATAGGGAAGAGTTTGTGGATGGATCATGG | 507 |
| 66944119 | 66805624 | CAGTGCATGGACAGTGATTAGCCCATAACA | |
| chrX: 66763874- | chrX: 66805745- | AAAGTTAAGGGTACCTTTTTTATATTTGCATCATATCTCCAGACCTTTTCCTTTATCTCCTTCTTGCAAGTTCTTCTTTCTTTCAGCTGA | 508 |
| 66944119 | 66805864 | CTATCTGCTGTTCCTGCTATGGCTCCCAGT | |
| chrX: 66763874- | chrX: 66805145- | AGCATAAACCTCAGTATTCTTTATCATTCAGTATCAACATTATTACTGAAAACAATAAGCAATATGGACTGAGTTTCTGTGGGGTGGAAA | 509 |
| 66944119 | 66805264 | TGTGAAGTGGATCATAGCATGATATAACTT | |
| chrX: 66763874- | chrX: 66805565- | GATAGGGAAGAGTTTGTGGATGGATCATGGCAGTGCATGGACAGTGATTAGCCCATAACACAACCAGTGAACACTGTTGTACCCAAAGCA | 510 |
| 66944119 | 66805684 | CATAAATCACCACATATACTATTAATATAT | |
| chrX: 66763874- | chrX: 66805985- | ATAGATATTTTGAGGAAGCATAATTTTCTATGTACCCCTCAAATCGTGGCTGGAGATGACAGCCTCTTCCACCTCCATATAAGACCATTT | 511 |
| 66944119 | 66806104 | CATTTCCTTCTACTTTTTTCTCCCTCCTTC | |
| chrX: 66763874- | chrX: 66805625- | CAACCAGTGAACACTGTTGTACCCAAAGCACATAAATCACCACATATACTATTAATATATTTATGGATGACAACAGACACTATAATTTTA | 512 |
| 66944119 | 66805744 | TGTCAGTGCTTTCTGCTGTGAAAAACAAAG | |
| chrX: 66763874- | chrX: 66805865- | GGCTTTTCAAGAGGGTACTTGTTTTTTAAGAGAAGACCCTTGAAGGACAGAGAGAGCCTGAATCATTCAAAATAATGAATTACTCAGGAT | 513 |
| 66944119 | 66805984 | GAAATTTCAATAATTTGCAAGTGTGTGGAG | |
| chrX: 66763874- | chrX: 66805445- | GTGAAGCCGAGGAATTCAAGGATTTGAGTCATGCCAGATTGCTCCATAACCATAGCCTATCTTTGTCACAAGTAAGAAGGTTTAAAAATC | 514 |
| 66944119 | 66805564 | ACCATACCATTATTGGTCACAACGTTTGGA | |
| chrX: 66763874- | chrX: 66805385- | CATATACCTGAAGTGAGAAGTCTGAGGTAACTTAGCAATAAGCTTGCAGTACAGTGTTTAGTGAAGCCGAGGAATTCAAGGATTTGAGTC | 515 |
| 66944119 | 66805504 | ATGCCAGATTGCTCCATAACCATAGCCTAT | |
| chrX: 66763874- | chrX: 66805685- | TTATGGATGACAACAGACACTATAATTTTATGTCAGTGCTTTCTGCTGTGAAAAACAAAGAAAGTTAAGGGTACCTTTTTTATATTTGCA | 516 |
| 66944119 | 66805804 | TCATATCTCCAGACCTTTTCCTTTATCTCC | |
| chrX: 66763874- | chrX: 66805325- | TCCGTAGTGAACATTATAACTCAAATGACTAGTCAGGTCTGTTCATTGCCCATGTAAAGGCATATACCTGAAGTGAGAAGTCTGAGGTAA | 517 |
| 66944119 | 66805444 | CTTAGCAATAAGCTTGCAGTACAGTGTTTA | |
| chrX: 66763874- | chrX: 66805805- | TTCTTGCAAGTTCTTCTTTCTTTCAGCTGACTATCTGCTGTTCCTGCTATGGCTCCCAGTGGCTTTTCAAGAGGGTACTTGTTTTTTAAG | 518 |
| 66944119 | 66805924 | AGAAGACCCTTGAAGGACAGAGAGAGCCTG | |
| chrX: 66763874- | chrX: 66805925- | AATCATTCAAAATAATGAATTACTCAGGATGAAATTTCAATAATTTGCAAGTGTGTGGAGATAGATATTTTGAGGAAGCATAATTTTCTA | 519 |
| 66944119 | 66806044 | TGTACCCCTCAAATCGTGGCTGGAGATGAC | |
| chrX: 66763874- | chrX: 66806622- | CAAACATAGTTAATGACCACTCCAGACCCAGTTGTTATAGAGTTGGCCCCAGCTGTATTGCTTCTATTTAAGACTAGGATAAGAAATGAC | 520 |
| 66944119 | 66806741 | ACTTTCCTACTTTTTACCTTATTGAAAGGG | |
| chrX: 66763874- | chrX: 66806442- | tGAATGACCTTCAACCTTTCAGGCTTCCAATTTTCTCTCTGAAAAGGACAGCCAAATGAAAACTCATAATTTTAGAAGATGAGGTTAGAC | 521 |
| 66944119 | 66806561 | GGTTGGTAGGTGCATGCAGAGACCAGTTAT | |
| chrX: 66763874- | chrX: 66806682- | CTTCTATTTAAGACTAGGATAAGAAATGACACTTTCCTACTTTTTACCTTATTGAAAGGGTAGAGGCTCACTGTTATCAATCTCAGTTCA | 522 |
| 66944119 | 66806801 | CTTGTTGATTGCACTGGCTTGCCAAGTGAG | |
| chrX: 66763874- | chrX: 66806502- | AACTCATAATTTTAGAAGATGAGGTTAGACGGTTGGTAGGTGCATGCAGAGACCAGTTATTATTTAGGTATTATGGAAGTTTATAGTTCT | 523 |
| 66944119 | 66806621 | TGTATGTTGAGTTCAGTGTAAGAGTGGCCC | |
| chrX: 66763874- | chrX: 66806562- | TATTTAGGTATTATGGAAGTTTATAGTTCTTGTATGTTGAGTTCAGTGTAAGAGTGGCCCCAAACATAGTTAATGACCACTCCAGACCCA | 524 |
| 6944119 | 66806681 | GTTGTTATAGAGTTGGCCCCAGCTGTATTG | |
| chrX: 66763874- | chrX: 66807163- | TCATTAATGTTCATTTCATTTATTTGGCTATCCATATGCTTTCCAGGGCGAAGGCAAGCTAGGACAAGGGCAGACAAGCAGCCTTAAAGT | 525 |
| 66944119 | 66807282 | TTGGGTGCTTTCCTTCGAAGTTGAGCTGCC | |
| chrX: 66763874- | chrX: 66807223- | AGGACAAGGGCAGACAAGCAGCCTTAAAGTTTGGGTGCTTTCCTTCGAAGTTGAGCTGCCTGTTTGAAAATCACACTTTTTGGTGATAGA | 526 |
| 66944119 | 66807342 | AGATGGTTCCAGTACAGATTTTATTTATTA | |
| chrX: 66763874- | chrX: 66807343- | CTGCATCTACATGGATAGACATTTTCCAAAGCATAGCTGAAAATATGTGTAAGTCCCAGAATATTTTCTGATTTAGACACAGACTTTGAG | 527 |
| 66944119 | 66807462 | CATGATAACCACATTTAGCATGTTAGGAAA | |
| chrX: 66763874- | chrX: 66807103- | GTTTGCCTAAATGTGTGCATCCTTGCTGGAAGCCAGCCATGATTCATGCTGCATAAGTATTCATTAATGTTCATTTCATTTATTTGGCTA | 528 |
| 66944119 | 66807222 | TCCATATGCTTTCCAGGGCGAAGGCAAGCT | |
| chrX: 66763874- | chrX: 66807283- | TGTTTGAAAATCACACTTTTTGGTGATAGAAGATGGTTCCAGTACAGATTTTATTTATTACTGCATCTACATGGATAGACATTTTCCAAA | 529 |
| 66944119 | 66807402 | GCATAGCTGAAAATATGTGTAAGTCCCAGA | |
| chrX: 66763874- | chrX: 66807043- | cacactgGACCAGCCAGGAGGCAGGAAAATCAGCTGGGGAATGTGGTGCCAACGTGTGATGTTTGCCTAAATGTGTGCATCCTTGCTGGA | 530 |
| 66944119 | 66807162 | AGCCAGCCATGATTCATGCTGCATAAGTAT | |
| chrX: 66763874- | chrX: 66807403- | ATATTTTCTGATTTAGACACAGACTTTGAGCATGATAACCACATTTAGCATGTTAGGAAATTCTGTCAGAATGCTTCTGGAAAGGCTACC | 531 |
| 66944119 | 66807522 | TTTCCAGAATGAAATGAAAAAAGaaaagga | |
| chrX: 66763874- | chrX: 66808336- | GCCTGTTTTTATTGGAAGAGCAAGAGAGAGAGGGAATGCTAGCTGGCAATTTCCCCAGGTACCCTTTATGAAAGTGCCCTTGGCTCTTCC | 532 |
| 66944119 | 66808455 | AATTTCATCTGAATAACCAGCTCAGGCAAA | |
| chrX: 66763874- | chrX: 66808216- | TCTCAAGGTATCCCAAAGCACTTTGTAAGGAAATATGACAAGCGCTGAGGCCATGCAGGCCAGTACAACAGCCGCCACCCAGCACTTCAC | 533 |
| 66944119 | 66808335 | AATTAGTCATGCCCAGCCTGGGATCATCAA | |
| chrX: 66763874- | chrX: 66808096- | TTGTCCTGGGAGCAGATTTCTCAAGCTGCCCATGTCCCCACACTGTTTGATTAAAAGGAGGTGCTTCAAACTCTTTGGCTTTATATAGAC | 534 |
| 66944119 | 66808215 | TAGAATCAGAATGATTGGTGGTGCCTCTGT | |
| chrX: 66763874- | chrX: 66808396- | ACCCTTTATGAAAGTGCCCTTGGCTCTTCCAATTTCATCTGAATAACCAGCTCAGGCAAATTTTCCTCTATCAAAAAGCAGAATGTGATA | 535 |
| 66944119 | 66808515 | GTGACAAGCTGATGCCCGGCTGATGCCCCA | |
| chrX: 66763874- | chrX: 66808576- | CCTTTTGTTCTTTTTCTGTGTTTCTTTCCCCATTGCCATCTGCAGAGTGTTCTCAGTCAGAAGTCAGCTGTGGGGTGGACAGTTTGTCAT | 536 |
| 66944119 | 66808695 | TTTAAGATCATCCCTATTCTGTCTACCTTT | |
| chrX: 66763874- | chrX: 66807796- | TGCTCCATGTTGAAATGCACTGCTCTTGCAAGGACTGATTATCTATTTTTCTGTCTTCCAAGGCCCCCTGTGTTCCACTCCACCCTCCCA | 537 |
| 66944119 | 66807915 | ATTCTGGGGGCTTCCAAAGTGGGCAGGTAC | |
| chrX: 66763874- | chrX: 66807976- | CTCTTTGCACACACTCCATATGAAGGCAAACTCCAGATCTTGGAGCCCATGTGTGTGTCATGCATTGTACTGCTTCTTGTACCCAAATCC | 538 |
| 66944119 | 66808095 | ATCTCAAGGGTGAGTAGACCAGGCTCAGAC | |
| chrX: 66763874- | chrX: 66807676- | CCACACCAGGAATTTAGAAGCTACTCAGTAAATATTGCTTCTCTCCTTTCCCCTTCCCCAGTCCTGTCCCCCGAGACATTCAGTAGTTAT | 539 |
| 66944119 | 66807795 | TCACAGGCATGCATTCTGAAGTCTGCCTAC | |
| chrX: 66763874- | chrX: 66808036- | TGCATTGTACTGCTTCTTGTACCCAAATCCATCTCAAGGGTGAGTAGACCAGGCTCAGACTTGTCCTGGGAGCAGATTTCTCAAGCTGCC | 540 |
| 66944119 | 66808155 | CATGTCCCCACACTGTTTGATTAAAAGGAG | |
| chrX: 66763874- | chrX: 66808456- | TTTTCCTCTATCAAAAAGCAGAATGTGATAGTGACAAGCTGATGCCCGGCTGATGCCCCAGGACATTGACTAAATAGACTTGGCCTCACA | 541 |
| 66944119 | 66808575 | ATTGGTTTTTATTCTCTATCTCCTTTCTTC | |
| chrX: 66763874- | chrX: 66808156- | GTGCTTCAAACTCTTTGGCTTTATATAGACTAGAATCAGAATGATTGGTGGTGCCTCTGTTCTCAAGGTATCCCAAAGCACTTTGTAAGG | 542 |
| 66944119 | 66808275 | AAATATGACAAGCGCTGAGGCCATGCAGGC | |
| chrX: 66763874- | chrX: 66807736- | GTCCTGTCCCCCGAGACATTCAGTAGTTATTCACAGGCATGCATTCTGAAGTCTGCCTACTGCTCCATGTTGAAATGCACTGCTCTTGCA | 543 |
| 66944119 | 66807855 | AGGACTGATTATCTATTTTTCTGTCTTCCA | |
| chrX: 66763874- | chrX: 66808636- | AAGTCAGCTGTGGGGTGGACAGTTTGTCATTTTAAGATCATCCCTATTCTGTCTACCTTTCTTATCCCTCATATCATTGCTTTTAGAGCA | 544 |
| 66944119 | 66808755 | AGGACAATTCTGGAAGTGAAACTACAATAA | |
| chrX: 66763874- | chrX: 66807856- | AGGCCCCCTGTGTTCCACTCCACCCTCCCAATTCTGGGGGCTTCCAAAGTGGGCAGGTACAGAATGTTCTGTGGAGCATCGGAGGCTGTT | 545 |
| 66944119 | 66807975 | ACTCAATATCTTGGCCAGCACTCTCAACTG | |
| chrX: 66763874- | chrX: 66808516- | GGACATTGACTAAATAGACTTGGCCTCACAATTGGTTTTTATTCTCTATCTCCTTTCTTCCCTTTTGTTCTTTTTCTGTGTTTCTTTCCC | 546 |
| 66944119 | 66808635 | CATTGCCATCTGCAGAGTGTTCTCAGTCAG | |
| chrX: 66763874- | chrX: 66808276- | CAGTACAACAGCCGCCACCCAGCACTTCACAATTAGTCATGCCCAGCCTGGGATCATCAAGCCTGTTTTTATTGGAAGAGCAAGAGAGAG | 547 |
| 66944119 | 66808395 | AGGGAATGCTAGCTGGCAATTTCCCCAGGT | |
| chrX: 66763874- | chrX: 66807916- | AGAATGTTCTGTGGAGCATCGGAGGCTGTTACTCAATATCTTGGCCAGCACTCTCAACTGCTCTTTGCACACACTCCATATGAAGGCAAA | 548 |
| 66944119 | 66808035 | CTCCAGATCTTGGAGCCCATGTGTGTGTCA | |
| chrX: 66763874- | chrX: 66809727- | ATTTTATATATCCCAATAGAGAAGCGTGGAAGACATCTAGGTTGCCACTGTCATTTGAAATTGGAATTTTTAAAAGAGAAACCTGAAGAC | 549 |
| 66944119 | 66809846 | TTGAAGAAAGCTTTCTTTTGCCTCCCCTTA | |
| chrX: 66763874- | chrX: 66809127- | aaagaaGTCCACTGTTAGTATCTTTTCCCCTGCCTAGTTTGTAAGCAACTGGCCTCTTCTATTTGTAAGTTACCTGTTTTCATTTCCATA | 550 |
| 66944119 | 66809246 | TGCCCCAAAGCAAACTTTAGCTCACGGCCT | |
| chrX: 66763874- | chrX: 66809187- | ATTTGTAAGTTACCTGTTTTCATTTCCATATGCCCCAAAGCAAACTTTAGCTCACGGCCTTACAGAGTGTGTATGTTAGTATGTTAAAAT | 551 |
| 66944119 | 66809306 | GAAATCAACTTTCCTCTCCCAGGCCTTCTA | |
| chrX: 66763874- | chrX: 66809667- | CAAGGGGAGGAGCTGTATACTCGAGCATGCCCTGTGGTTCCTGGCCCTGACTGAGGGACTATTTTATATATCCCAATAGAGAAGCGTGGA | 552 |
| 66944119 | 66809786 | AGACATCTAGGTTGCCACTGTCATTTGAAA | |
| chrX: 66763874- | chrX: 66809247- | TACAGAGTGTGTATGTTAGTATGTTAAAATGAAATCAACTTTCCTCTCCCAGGCCTTCTAATTGACATGAATTTGGGAGTAGACTTGCAT | 553 |
| 66944119 | 66809366 | TGGCCTTTGTCCTGACAGCCAACAGAGTCC | |
| chrX: 66763874- | chrX: 66809607- | CAGCCAGTTTCCCCTCTGTTTTCTCCCTTGCTTACACAGCCAAGGAGTGGAGCCAAGCCTCAAGGGGAGGAGCTGTATACTCGAGCATGC | 554 |
| 66944119 | 66809726 | CCTGTGGTTCCTGGCCCTGACTGAGGGACT | |
| chrX: 66763874- | chrX: 66809967- | GCTTCAACATTGCTCACCCTGACCAGGTATGAAGCCAAGAGTTTGGTTTAGGGCATAAAAGAATGTCGGAACTCAAGGACTAGGTTGAGG | 555 |
| 66944119 | 66810086 | TGGGGAAGGGGGATGAAGGCTTCTTTTTTT | |
| chrX: 66763874- | chrX: 66809427- | TATACATTTTGAGGGCAGACTCAACTTGAGTAAACCTGATTGAGCTTTCCCCATCTGCCTCCCAGAGATCACTGCCTGTGCTTTGTTAAA | 556 |
| 66944119 | 66809546 | AAGAGAATTATAGGAGTCCTCTCAAGGCAG | |
| chrX: 66763874- | chrX: 66809307- | ATTGACATGAATTTGGGAGTAGACTTGCATTGGCCTTTGTCCTGACAGCCAACAGAGTCCTCTTCTGTTGTATTCACTGTTGCCTTCCAT | 557 |
| 66944119 | 66809426 | GAGGATCCCATGGAGAAAGTTTGTCATTGA | |
| chrX: 66763874- | chrX: 66809367- | TCTTCTGTTGTATTCACTGTTGCCTTCCATGAGGATCCCATGGAGAAAGTTTGTCATTGATATACATTTTGAGGGCAGACTCAACTTGAG | 558 |
| 66944119 | 66809486 | TAAACCTGATTGAGCTTTCCCCATCTGCCT | |
| chrX: 66763874- | chrX: 66809787- | TTGGAATTTTTAAAAGAGAAACCTGAAGACTTGAAGAAAGCTTTCTTTTGCCTCCCCTTACAGTTGATTTTTGAGCTTCTTAAAGCTACC | 559 |
| 66944119 | 66809906 | TAGTCCAAAGTACCCACACTCTTATTCTTT | |
| chrX: 66763874- | chrX: 66809487- | CCCAGAGATCACTGCCTGTGCTTTGTTAAAAAGAGAATTATAGGAGTCCTCTCAAGGCAGAGAGGCCTAAAATTAGACATGGCAGCCATG | 560 |
| 66944119 | 66809606 | CCTTTGGTGTGCATGGAGGTTGGATACAGG | |
| chrX: 66763874- | chrX: 66809847- | CAGTTGATTTTTGAGCTTCTTAAAGCTACCTAGTCCAAAGTACCCACACTCTTATTCTTTTGTCTTTCCTACTGGTTTTATTTTTTTTTC | 561 |
| 66944119 | 66809966 | ATCTTCCCAGGTGTTTGATGATCACTAAGA | |
| chrX: 66763874- | chrX: 66809907- | TGTCTTTCCTACTGGTTTTATTTTTTTTTCATCTTCCCAGGTGTTTGATGATCACTAAGAGCTTCAACATTGCTCACCCTGACCAGGTAT | 562 |
| 66944119 | 66810026 | GAAGCCAAGAGTTTGGTTTAGGGCATAAAA | |
| chrX: 66763874- | chrX: 66810027- | GAATGTCGGAACTCAAGGACTAGGTTGAGGTGGGGAAGGGGGATGAAGGCTTCTTTTTTTCTTGGGTTAAGCAGAAATAACTTAGATCTC | 563 |
| 66944119 | 66810146 | AGAGTGAAAGCCTTGAATTATCACATATAT | |
| chrX: 66763874- | chrX: 66809547- | AGAGGCCTAAAATTAGACATGGCAGCCATGCCTTTGGTGTGCATGGAGGTTGGATACAGGCAGCCAGTTTCCCCTCTGTTTTCTCCCTTG | 564 |
| 66944119 | 66809666 | CTTACACAGCCAAGGAGTGGAGCCAAGCCT | |
| chrX: 66763874- | chrX: 66810087- | CTTGGGTTAAGCAGAAATAACTTAGATCTCAGAGTGAAAGCCTTGAATTATCACATATATCACTGGAAAAGACTAGTTCTTTGCTATGAT | 565 |
| 66944119 | 66810206 | AACAATTGTTCATCATCTCTCCCCTgagga | |
| chrX: 66763874- | chrX: 66810986- | GGGCTAGAAGTAAGCATGCTACTAGAAACAGAATTTGGGAACACAGCTCTGGGCCTAGAAAAGCGACCTGTCAACTTGTTACAGTTAACA | 566 |
| 66944119 | 66811105 | TCAATAACTATAGGATGGGTTTGGTGGAAA | |
| chrX: 66763874- | chrX: 66810746- | AAATTGGCTGCGTTCTGAATCCTATTTTTATTTGGGATAACAATAAGCCTGTATGGTCACTGTGACCTTTGATTTGCTGTTTCTGCAACC | 567 |
| 66944119 | 66810865 | TCACACTTGTCTCAGGATTCTTCTTCCACT | |
| chrX: 66763874- | chrX: 66810806- | TGTGACCTTTGATTTGCTGTTTCTGCAACCTCACACTTGTCTCAGGATTCTTCTTCCACTTCTGCACTTTATATTGGGTTTCTTCCAGGC | 568 |
| 66944119 | 66810925 | ATCATATTAAACTTTAAGCCAGGTATGTGT | |
| chrX: 66763874- | chrX: 66810866- | TCTGCACTTTATATTGGGTTTCTTCCAGGCATCATATTAAACTTTAAGCCAGGTATGTGTATATGCATGGGCTGTGGGCCTGAAAAAAAT | 569 |
| 66944119 | 66810985 | TAGCCCGAGAGAGAAAAAAATTTAAGTAGT | |
| chrX: 66763874- | chrX: 66810926- | ATATGCATGGGCTGTGGGCCTGAAAAAAATTAGCCCGAGAGAGAAAAAAATTTAAGTAGTGGGCTAGAAGTAAGCATGCTACTAGAAACA | 570 |
| 66944119 | 66811045 | GAATTTGGGAACACAGCTCTGGGCCTAGAA | |
| chrX: 66763874- | chrX: 66810566- | gTAAACTATACTTTCTCCTTTCCAGACTATCCTAGTAAGAAAATTCTCTTTTAAGACAGAGTAGAACTCTGGAATTCATCAGTTTTGATG | 571 |
| 66944119 | 66810685 | TTTCTTAAAGTGTAATCTAAGATAGTGCTC | |
| chrX: 66763874- | chrX: 66810686- | CTGTATTAAGTTCTGATGTCTGACCATTGTTCAAATAAAGAGTAAAATGCAAATGACAGGAAATTGGCTGCGTTCTGAATCCTATTTTTA | 572 |
| 66944119 | 66810805 | TTTGGGATAACAATAAGCCTGTATGGTCAC | |
| chrX: 66763874- | chrX: 66810626- | GTAGAACTCTGGAATTCATCAGTTTTGATGTTTCTTAAAGTGTAATCTAAGATAGTGCTCCTGTATTAAGTTCTGATGTCTGACCATTGT | 573 |
| 66944119 | 66810745 | TCAAATAAAGAGTAAAATGCAAATGACAGG | |
| chrX: 66763874- | chrX: 66811046- | AAGCGACCTGTCAACTTGTTACAGTTAACATCAATAACTATAGGATGGGTTTGGTGGAAAATTATGCTGACCAACAGGGTGGGAGAGAAT | 574 |
| 66944119 | 66811165 | AGGGTCAGAATATATATCGCTGTAAGGTTg | |
| chrX: 66763874- | chrX: 66811246- | AACTGGAGAGCTTGACTTCTCTTGAATATATTTTTAAATAAAGTACTCCTTTCAACTCCAAATGCAGCAGGCTTGGTTCCCTTCTCCTAC | 575 |
| 66944119 | 66811365 | CTCCATTGCGGATGAAAGCTTAATCTTTAA | |
| chrX: 66763874- | chrX: 66811606- | GTTGCAGCACTGTCATTCTCCAGTTTCTCTTGGAGTTGTCACCACCCTCTCCTTTGTTCTCACTGCTGACATCATTTGTAAAATAATTTC | 576 |
| 66944119 | 66811725 | TTCCCTTAAATAAACAAGACATACAATCCT | |
| chrX: 66763874- | chrX: 66811426- | TAAGGCCCAACAGGGCTCTCCAAGGAGGCAAAATTCTGATGATACATTTCTGTTTAGTGGAAAATGGGTAGGGAAAATTATGTCTTAGAA | 577 |
| 66944119 | 66811545 | TCAATTAACCAAACATAAAATCCTCCAAGG | |
| chrX: 66763874- | chrX: 66811306- | AATGCAGCAGGCTTGGTTCCCTTCTCCTACCTCCATTGCGGATGAAAGCTTAATCTTTAAGATGGGCTTGGGTGGGTAGAGTACGCCCCT | 578 |
| 66944119 | 66811425 | TGGTGAGCACTGTGCTCTCTGCAACCCCAA | |
| chrX: 66763874- | chrX: 66812266- | ATTTAAAATATGATTTGATTATGTACATTTCAGATTTTTCTACCTTTCTAGGAGTATCTCTGTTGTATAAAAACACAAAATTCTGGAACT | 579 |
| 66944119 | 66812385 | TTTGAAAGGAAGATGTGCCTCTCTTCATAC | |
| chrX: 66763874- | chrX: 66811666- | CACTGCTGACATCATTTGTAAAATAATTTCTTCCCTTAAATAAACAAGACATACAATCCTCTAAATGACTAAAGAACAGTTACCTAGAAG | 580 |
| 66944119 | 66811785 | AAACCTTAGTGGAAAGTATTTTCTTCATCT | |
| chrX: 66763874- | chrX: 66812206- | TTTCACTTCCTAAAAAGATTTTGAATAAGATGTCTTTTAAGTAAGAAGCTCCCTGAATGCATTTAAAATATGATTTGATTATGTACATTT | 581 |
| 66944119 | 66812325 | CAGATTTTTCTACCTTTCTAGGAGTATCTC | |
| chrX: 66763874- | chrX: 66812146- | GCACAATATAGGGCAATCCAGGTTTACACAAAGGATTAATTTGGGAACAATTATCCTCATTTTCACTTCCTAAAAAGATTTTGAATAAGA | 582 |
| 66944119 | 66812265 | TGTCTTTTAAGTAAGAAGCTCCCTGAATGC | |
| chrX: 66763874- | chrX: 66812026- | TGTGGGGATGTGAGGATCTATGTCTACCAATTGCAGCCTCTGCTGCAAATTGGAGGCAGAAATCTGGGCTGAACAATAGGTAAGAGTGTC | 583 |
| 66944119 | 66812145 | AACTCTACAGATCTCTCACATGCTAAGCAA | |
| chrX: 66763874- | chrX: 66812086- | AATCTGGGCTGAACAATAGGTAAGAGTGTCAACTCTACAGATCTCTCACATGCTAAGCAAGCACAATATAGGGCAATCCAGGTTTACACA | 584 |
| 66944119 | 66812205 | AAGGATTAATTTGGGAACAATTATCCTCAT | |
| chrX: 66763874- | chrX: 66811966- | GGCCTCTTAATAAAGAACTTGTGGTTTGAGTGTTCATTGAAATTAGCCATATTAGGTTTATGTGGGGATGTGAGGATCTATGTCTACCAA | 585 |
| 66944119 | 66812085 | TTGCAGCCTCTGCTGCAAATTGGAGGCAGA | |
| chrX: 66763874- | chrX: 66811546- | GGCTTGGTAGGATGCCTAGGGAAGAGCCACGAGATAAAAACTCCAGGCTGGAAGGGCATTGTTGCAGCACTGTCATTCTCCAGTTTCTCT | 586 |
| 66944119 | 66811665 | TGGAGTTGTCACCACCCTCTCCTTTGTTCT | |
| chrX: 66763874- | chrX: 66811846- | AAGAGATGCATGCTGACTTAAAAGGCATGATATATGTGAAACTAAGATAATGTGTTCAAGAGTGATGCTTTGTTGATGCAGAACCACTGA | 587 |
| 66944119 | 66811965 | ATTCCTTACTATTATGTTTGCCTGACTATC | |
| chrX: 66763874- | chrX: 66812326- | TGTTGTATAAAAACACAAAATTCTGGAACTTTTGAAAGGAAGATGTGCCTCTCTTCATACATTTGTCATTCTTGAACGATTGTAAAATGA | 588 |
| 66944119 | 66812445 | AGTGACTGCATATCACGTCATGTGCCCTAT | |
| chrX: 66763874- | chrX: 66811726- | CTAAATGACTAAAGAACAGTTACCTAGAAGAAACCTTAGTGGAAAGTATTTTCTTCATCTAACGGATGATTGTCTTTACAGAGGTGGAGT | 589 |
| 66944119 | 66811845 | AAAGGATGTGCGAGGGAGCATAATCAAGCT | |
| chrX: 66763874- | chrX: 66811786- | AACGGATGATTGTCTTTACAGAGGTGGAGTAAAGGATGTGCGAGGGAGCATAATCAAGCTAAGAGATGCATGCTGACTTAAAAGGCATGA | 590 |
| 66944119 | 66811905 | TATATGTGAAACTAAGATAATGTGTTCAAG | |
| chrX: 66763874- | chrX: 66811366- | GATGGGCTTGGGTGGGTAGAGTACGCCCCTTGGTGAGCACTGTGCTCTCTGCAACCCCAATAAGGCCCAACAGGGCTCTCCAAGGAGGCA | 591 |
| 66944119 | 66811485 | AAATTCTGATGATACATTTCTGTTTAGTGG | |
| chrX: 66763874- | chrX: 66811486- | AAAATGGGTAGGGAAAATTATGTCTTAGAATCAATTAACCAAACATAAAATCCTCCAAGGGGCTTGGTAGGATGCCTAGGGAAGAGCCAC | 592 |
| 66944119 | 66811605 | GAGATAAAAACTCCAGGCTGGAAGGGCATT | |
| chrX: 66763874- | chrX: 66811906- | AGTGATGCTTTGTTGATGCAGAACCACTGAATTCCTTACTATTATGTTTGCCTGACTATCGGCCTCTTAATAAAGAACTTGTGGTTTGAG | 593 |
| 66944119 | 66812025 | TGTTCATTGAAATTAGCCATATTAGGTTTA | |
| chrX: 66763874- | chrX: 66812548- | TAAGTTATGTCAATATCTATTTATAAAGATTTTTGTGATATTCTTTTCACTGTAGAACTTCAAGCATATCCTAAAAGGAACGGTTAGATA | 594 |
| 66944119 | 66812667 | CCTCTACAAACTGTGGCAATGACTTACTGA | |
| chrX: 66763874- | chrX: 66812728- | GTTTCAGAAGAGCAAAACTAAGACAATCCAGGGAAATGCCATTTGAGAATTTCTAACTTTAAAAAAACAAGTAAAATAGTGCCAAGAATA | 595 |
| 66944119 | 66812847 | TTATCTAACTAACCCCAAAGTCTACAATGT | |
| chrX: 66763874- | chrX: 66812668- | GTAATTGCTGGCAACTGATTTTTGGTGCTTCTTGTTTTGATAGTATAGCAGTGCGAGTAGGTTTCAGAAGAGCAAAACTAAGACAATCCA | 596 |
| 66944119 | 66812787 | GGGAAATGCCATTTGAGAATTTCTAACTTT | |
| chrX: 66763874- | chrX: 66812848- | AACTCTTTTATTTTGATAATGCTGTTCTAACCCTATCTACTTCAGTCCTTTCCCACCCAGCTGGTTTAGGAATCAAATTCCCAATGTTTC | 597 |
| 66944119 | 66812967 | ATCACTGTTAACATTACTGTTTTACTCTTC | |
| chrX: 66763874- | chrX: 66812908- | CTGGTTTAGGAATCAAATTCCCAATGTTTCATCACTGTTAACATTACTGTTTTACTCTTCACTTTAGTTCTTAAATGGCATAGTGTCTTA | 598 |
| 66944119 | 66813027 | AATTCCCTCAGCCTCTTTCACATTTGATTT | |
| chrX: 66763874- | chrX: 66812788- | AAAAAAACAAGTAAAATAGTGCCAAGAATATTATCTAACTAACCCCAAAGTCTACAATGTAACTCTTTTATTTTGATAATGCTGTTCTAA | 599 |
| 66944119 | 66812907 | CCCTATCTACTTCAGTCCTTTCCCACCCAG | |
| chrX: 66763874- | chrX: 66813028- | CTTTGGAAACTTTTTACCTTTTCATTGAAGCCCATATGATCTTTTCCGAAACAGACCCTTATCTTTACCTCCTTCTTTGGAGTCTTTCTC | 600 |
| 66944119 | 66813147 | CTACTTGAATTTCTGAACTTCTTAAAATGG | |
| chrX: 66763874- | chrX: 66812968- | ACTTTAGTTCTTAAATGGCATAGTGTCTTAAATTCCCTCAGCCTCTTTCACATTTGATTTCTTTGGAAACTTTTTACCTTTTCATTGAAG | 601 |
| 66944119 | 66813087 | CCCATATGATCTTTTCCGAAACAGACCCTT | |
| chrX: 66763874- | chrX: 66812608- | CAAGCATATCCTAAAAGGAACGGTTAGATACCTCTACAAACTGTGGCAATGACTTACTGAGTAATTGCTGGCAACTGATTTTTGGTGCTT | 602 |
| 66944119 | 66812727 | CTTGTTTTGATAGTATAGCAGTGCGAGTAG | |
| chrX: 66763874- | chrX: 66813550- | ACTATTTTGTCATACAGGCAACAGAATGTTAAACCATTTCATAAAACAATGACAAATATACATGAATTTTTCATCAGTTATAAATGCATT | 603 |
| 66944119 | 66813669 | TCCTTTATAACATTGAACATGTTTTTGCAA | |
| chrX: 66763874- | chrX: 66813730- | TTAATTTTTTCAGATTAATTTTTCAGAAAAGTGACTGTTTCTGTCTATTGTCTTAACCCCAGGCATCAAAGGATTTTAATCAGAAAGAAC | 604 |
| 66944119 | 66813849 | CGAGGAATAATTTGGTTATTTTAGTGCCtt | |
| chrX: 66763874- | chrX: 66813610- | CATGAATTTTTCATCAGTTATAAATGCATTTCCTTTATAACATTGAACATGTTTTTGCAACTGAAATAAGTACGGTTTTCATTTTTAGAA | 605 |
| 66944119 | 66813729 | GGCACATGATAAAGTTAAGGCAGTGGTTAA | |
| chrX: 66763874- | chrX: 66813670- | CTGAAATAAGTACGGTTTTCATTTTTAGAAGGCACATGATAAAGTTAAGGCAGTGGTTAATTAATTTTTTCAGATTAATTTTTCAGAAAA | 606 |
| 66944119 | 66813789 | GTGACTGTTTCTGTCTATTGTCTTAACCCC | |
| chrX: 66763874- | chrX: 66813490- | ccAGTAATAAGTTTTCTTAAGTGCTTTCTTAATATTCTGATATTTTTAAAAAAGATCTGGACTATTTTGTCATACAGGCAACAGAATGTT | 607 |
| 66944119 | 66813609 | AAACCATTTCATAAAACAATGACAAATATA | |
| chrX: 66763874- | chrX: 66814132- | acccTATTTTAGAGCTTTGTCAAGCTTTGGAAAGAAAACCATTTATAATATAATAGATAAATTATGGATATTTGAGGCAGTTTTTATCAT | 608 |
| 66944119 | 66814251 | AGTATACATGGTAAACCACAGCCCCCCttt | |
| chrX: 66763874- | chrX: 66814283- | ttaCTTTTATCTTAAACATGTTTTAACAAAGCAAGCATATGTAGATTAGCACTAATTAAAACAAAAACCTTTGTAATGATAGCTGTTTTT | 609 |
| 66944119 | 66814402 | TATATGATTACAAAAAATTTACTATACaaa | |
| chrX: 66763874- | chrX: 66815124- | ACTGTATATTGACAAATTTAGCAACAAAATGAGCTTGAGAAAAAAATCAAGGCCTGCCATGGCATCTTTGCTTTTTTTTCTTAAAAAAAA | 610 |
| 66944119 | 66815243 | AACTTTTTAGAAAGATTATGCGACTGTATT | |
| chrX: 66763874- | chrX: 66815184- | GGCATCTTTGCTTTTTTTTCTTAAAAAAAAAACTTTTTAGAAAGATTATGCGACTGTATTATCTGTAACTACTGCAATGGTGTAAATCCT | 611 |
| 66944119 | 66815303 | GATGGTATAATTTGCTTTTTAAAGCTATCT | |
| chrX: 66763874- | chrX: 66815064- | GATGAGAGCTGTGTCACAGCTAATTTTTCTTTAGTAATTAAAGGTTTATAAAAATCTTACACTGTATATTGACAAATTTAGCAACAAAAT | 612 |
| 66944119 | 66815183 | GAGCTTGAGAAAAAAATCAAGGCCTGCCAT | |
| chrX: 66763874- | chrX: 66814884- | GGAAGACAGTTACCATTTCAGATCGGCAGAAGTTGTGGCTTTAATCTAGACTCGAATATGTTTTACATCAAAGGGTTGCCTCAACAGTGC | 613 |
| 66944119 | 66815003 | TCAAACCTGCCTCTCTGAAAACATGCTGAG | |
| chrX: 66763874- | chrX: 66814944- | TTTTACATCAAAGGGTTGCCTCAACAGTGCTCAAACCTGCCTCTCTGAAAACATGCTGAGCACGAAGGTTACTTGAAGTCTTAGCTTGAG | 614 |
| 66944119 | 66815063 | TACTTAAGAGAGTGCTATGGAGGGATTGTT | |
| chrX: 66763874- | chrX: 66815004- | CACGAAGGTTACTTGAAGTCTTAGCTTGAGTACTTAAGAGAGTGCTATGGAGGGATTGTTGATGAGAGCTGTGTCACAGCTAATTTTTCT | 615 |
| 66944119 | 66815123 | TTAGTAATTAAAGGTTTATAAAAATCTTAC | |
| chrX: 66763874- | chrX: 66814824- | GCTCCTCCGCTTTTTCAGTTCCTAAATAATTTTCCAATCTAGAATGCAAAAGATTCTGAAGGAAGACAGTTACCATTTCAGATCGGCAGA | 616 |
| 66944119 | 66814943 | AGTTGTGGCTTTAATCTAGACTCGAATATG | |
| chrX: 66763874- | chrX: 66816352- | TCTATCTTGGCCTCCATATGCAATCAAATAAGAAACACATTTTAAGCATATTATTTACCTTGTGGATTCTGCCTTCCTCAGTGTGTTCAG | 617 |
| 66944119 | 66816471 | TCTGTGTATATTCATTTCTCCCACACTGTA | |
| chrX: 66763874- | chrX: 66816292- | CATTTTAAAGTCTCATTTTAGATTATTTTGACTATCTGATTTTTAAAATGGTTTAAAAAATCTATCTTGGCCTCCATATGCAATCAAATA | 618 |
| 66944119 | 66816411 | AGAAACACATTTTAAGCATATTATTTACCT | |
| chrX: 66763874- | chrX: 66816532- | AAGCATACATAGACTAGTGAGCACCACTCATTACATGTCATTTCTCTAGAGAAACTAGTTGGGCCATGGCTGCAGGACTCTCACTTGAAA | 619 |
| 66944119 | 66816651 | AGACATGTGTGGTGATGTTTTCTCAGGCAG | |
| chrX: 66763874- | chrX: 66816652- | TTAAGCAATAAAGTGTACCCTGATTTGCACTGAAAATAAAGATTCCTTTAAAGGGAGCAGTTCTAGTTATCTCTCTCTTTAGGTACCATA | 620 |
| 66944119 | 66816771 | TGCTGAACGTTTTTCTATGCACTAAAACAG | |
| chrX: 66763874- | chrX: 66816232- | TTATTTCCTTTGTTTTTCAATTGATATTTACTCTTAAATTAAACTAATTATTTAAAAAAGCATTTTAAAGTCTCATTTTAGATTATTTTG | 621 |
| 66944119 | 66816351 | ACTATCTGATTTTTAAAATGGTTTAAAAAA | |
| chrX: 66763874- | chrX: 66816172- | AGGGTCTTCTTGGTGACTCCACTTCTTTTCTTTGTTTATGTATCCTTCCAGATGATGTATTTATTTCCTTTGTTTTTCAATTGATATTTA | 622 |
| 66944119 | 66816291 | CTCTTAAATTAAACTAATTATTTAAAAAAG | |
| chrX: 66763874- | chrX: 66816412- | TGTGGATTCTGCCTTCCTCAGTGTGTTCAGTCTGTGTATATTCATTTCTCCCACACTGTAAGAAGCTAGTCAGATGTATAATTGGATTAT | 623 |
| 66944119 | 66816531 | CATGCTACATAATCTTAGCACACTCATTTT | |
| chrX: 66763874- | chrX: 66816592- | GGGCCATGGCTGCAGGACTCTCACTTGAAAAGACATGTGTGGTGATGTTTTCTCAGGCAGTTAAGCAATAAAGTGTACCCTGATTTGCAC | 624 |
| 66944119 | 66816711 | TGAAAATAAAGATTCCTTTAAAGGGAGCAG | |
| chrX: 66763874- | chrX: 66816472- | AGAAGCTAGTCAGATGTATAATTGGATTATCATGCTACATAATCTTAGCACACTCATTTTAAGCATACATAGACTAGTGAGCACCACTCA | 625 |
| 66944119 | 66816591 | TTACATGTCATTTCTCTAGAGAAACTAGTT | |
| chrX: 66763874- | chrX: 66817409- | TCTAGCCCCACCTTTGGGCCCCTTCATAAGCAAACATGCAGGTTTTCCAGAGAGCTGTATGCTACTGAATGCAGAAAATTTGGCTCATAC | 626 |
| 66944119 | 66817528 | TGGCCTATGGACTATCTGCTCACTGCCCTG | |
| chrX: 66763874- | chrX: 66817169- | AGCTCTTGAACTGAGCTCTTTAACTGACCTCAGCTCTTGAACTATGGTACAAGATCCCATGGTCCTGTTTGGTACCTCCATTTGCCCTCC | 627 |
| 66944119 | 66817288 | TTTTCACTCTCTGGGAGCATAGCTAAGTTC | |
| chrX: 66763874- | chrX: 66817649- | CTGGAAAACGAAAAATAGAATTATCCAACTACCACTTTAAAATTGGACAAAGACTTTTGTTGTTGTTGTTGGAGGGGGTGGTAAACATCA | 628 |
| 66944119 | 66817768 | TTTTAGCAGACCAAATATACTTTTGGTGAA | |
| chrX: 66763874- | chrX: 66817469- | GCTACTGAATGCAGAAAATTTGGCTCATACTGGCCTATGGACTATCTGCTCACTGCCCTGATAACTATTTTCCAAGGGAGTGGGTGCCCT | 629 |
| 66944119 | 66817588 | ACCTTTCCTACATGAAGTTTTTTGCTAGTC | |
| chrX: 66763874- | chrX: 66817589- | TTGCCCTAAAAATTCTAGGTATCCCTTGCTTTTAGGATAAATATGTTTCACTGGGACCAGCTGGAAAACGAAAAATAGAATTATCCAACT | 630 |
| 66944119 | 66817708 | ACCACTTTAAAATTGGACAAAGACTTTTGT | |
| chrX: 66763874- | chrX: 66817529- | ATAACTATTTTCCAAGGGAGTGGGTGCCCTACCTTTCCTACATGAAGTTTTTTGCTAGTCTTGCCCTAAAAATTCTAGGTATCCCTTGCT | 631 |
| 66944119 | 66817648 | TTTAGGATAAATATGTTTCACTGGGACCAG | |
| chrX: 66763874- | chrX: 66817229- | GGTCCTGTTTGGTACCTCCATTTGCCCTCCTTTTCACTCTCTGGGAGCATAGCTAAGTTCAAAATTGAATTAGGTACTTGTAGTAAGAGC | 632 |
| 66944119 | 66817348 | ATACTTATAATCCTGGGATCTTCATGTTGC | |
| chrX: 66763874- | chrX: 66817349- | CAGATATTAACCTCTTGAAGTTTTTCACCACAACCTGGGCACTTTTCTGATTTGCTCACTTCTAGCCCCACCTTTGGGCCCCTTCATAAG | 633 |
| 66944119 | 66817468 | CAAACATGCAGGTTTTCCAGAGAGCTGTAT | |
| chrX: 66763874- | chrX: 66817289- | AAAATTGAATTAGGTACTTGTAGTAAGAGCATACTTATAATCCTGGGATCTTCATGTTGCCAGATATTAACCTCTTGAAGTTTTTCACCA | 634 |
| 66944119 | 66817408 | CAACCTGGGCACTTTTCTGATTTGCTCACT | |
| chrX: 66763874- | chrX: 66817997- | CACTGCTCCTTATGAAAATGAGGAAATAATTGAGATGAGTGAGCCATTGAGGCAACAGTACAAAAAGTGCTGAAAACTCACTGCTTAAAT | 635 |
| 66944119 | 66818116 | AAGCACCTCTTACTGCTTTTGTGGCACTTT | |
| chrX: 66763874- | chrX: 66818857- | GAGATGAGAAGTAGGAGGTGGAGAGAACTAACCAGAAGAGGGTACCCAAATAAACCAGAAATATGTATGTGTTAGAGAAGGGGCCTATTG | 636 |
| 66944119 | 66818976 | AGCGGGTGGCAGTGGCATGTGTGGCATTAC | |
| chrX: 66763874- | chrX: 66818437- | ATTCTGACTAGAAAAGTAATGTTTGGTTTTGTTTGGCTCTTTGCTTAATATACCCATAATAAGGGTACCTATTTGCCTTTGGACCATTAG | 637 |
| 66944119 | 66818556 | TTCAAATATTATTTTATTAATATGGAATTA | |
| chrX: 66763874- | chrX: 66818557- | CTGGGCTCCAGAAGCCATAGTCTTCTTAGCTGCTCCCTATCCCCACTCTCACCTCAATTTTTTTTTTTCACTTTTGTTTTTCTTCTCAGG | 638 |
| 66944119 | 66818676 | GAAAGGTTTGAGGCAAAGAATGTCTTCTTA | |
| chrX: 66763874- | chrX: 66818737- | ATGGACATGGTAGAATCTTTGTCCTGAGGGAGCTATCTAGATCCATTCCTTCTGATATGCAGCCAGTAGCCACTTGTGGTAATGGAGCAA | 639 |
| 66944119 | 66818856 | TAGAAACAACACTAGTTCAAGTGGAAACGT | |
| chrX: 66763874- | chrX: 66818917- | ATATGTATGTGTTAGAGAAGGGGCCTATTGAGCGGGTGGCAGTGGCATGTGTGGCATTACTTGCTCCTGTATTCTCTGCTTTTTACTTAG | 640 |
| 66944119 | 66819036 | TTGTGGCTTTGGTGGTATAGTCTCAAATCT | |
| chrX: 66763874- | chrX: 66818677- | TGATCCAAAACCAAGCATGGTGGTGATTTATTCACCAAGAGATTCCTAAGTACCTGTGTGATGGACATGGTAGAATCTTTGTCCTGAGGG | 641 |
| 66944119 | 66818796 | AGCTATCTAGATCCATTCCTTCTGATATGC | |
| chrX: 66763874- | chrX: 66819037- | AAGTTACGTAGGTAATATTGTTATGTATCATGTTTTGGCAATGTAGACTAAATACTTGCTCATAAGAGTACAGGACAATGAGGATAGTTT | 642 |
| 66944119 | 66819156 | GGTTTTGTTTACTGCATGGAAAATGCAGGA | |
| chrX: 66763874- | chrX: 66818497- | AAGGGTACCTATTTGCCTTTGGACCATTAGTTCAAATATTATTTTATTAATATGGAATTACTGGGCTCCAGAAGCCATAGTCTTCTTAGC | 643 |
| 66944119 | 66818616 | TGCTCCCTATCCCCACTCTCACCTCAATTT | |
| chrX: 66763874- | chrX: 66818797- | AGCCAGTAGCCACTTGTGGTAATGGAGCAATAGAAACAACACTAGTTCAAGTGGAAACGTGAGATGAGAAGTAGGAGGTGGAGAGAACTA | 644 |
| 66944119 | 66818916 | ACCAGAAGAGGGTACCCAAATAAACCAGAA | |
| chrX: 66763874- | chrX: 66818617- | TTTTTTTTCACTTTTGTTTTTCTTCTCAGGGAAAGGTTTGAGGCAAAGAATGTCTTCTTATGATCCAAAACCAAGCATGGTGGTGATTTA | 645 |
| 66944119 | 66818736 | TTCACCAAGAGATTCCTAAGTACCTGTGTG | |
| chrX: 66763874- | chrX: 66819097- | CATAAGAGTACAGGACAATGAGGATAGTTTGGTTTTGTTTACTGCATGGAAAATGCAGGATGTTTAGTAAATAGATTCATGGCGTAGTGA | 646 |
| 66944119 | 66819216 | GTTCACTACTAAAATCAGACTCTGAGAATG | |
| chrX: 66763874- | chrX: 66818977- | TTGCTCCTGTATTCTCTGCTTTTTACTTAGTTGTGGCTTTGGTGGTATAGTCTCAAATCTAAGTTACGTAGGTAATATTGTTATGTATCA | 647 |
| 66944119 | 66819096 | TGTTTTGGCAATGTAGACTAAATACTTGCT | |
| chrX: 66763874- | chrX: 66819157- | TGTTTAGTAAATAGATTCATGGCGTAGTGAGTTCACTACTAAAATCAGACTCTGAGAATGGGTTTGATTTAAATGGCTAGTTTAGAAGAC | 648 |
| 66944119 | 66819276 | TGAATTTAGGCCACTTGATTGAGAAAGGCC | |
| chrX: 66763874- | chrX: 66819504- | AACAAATGAATACAATTTTAGAGTAAATTTTTACTGCTTACACTACATGTAGATTTTCTTTTTAGAGATTTCGCAATGCTGATTTATTTC | 649 |
| 66944119 | 66819623 | AAAATAAGCTTGAAGCTAAGCGACAAAGCT | |
| chrX: 66763874- | chrX: 66819444- | tgctcaatcaatgttgattCTATGCTACCAACAAAAATGAGTCCATGATGTTTACTATTCAACAAATGAATACAATTTTAGAGTAAATTT | 650 |
| 66944119 | 66819563 | TTACTGCTTACACTACATGTAGATTTTCTT | |
| chrX: 66763874- | chrX: 66819564- | TTTAGAGATTTCGCAATGCTGATTTATTTCAAAATAAGCTTGAAGCTAAGCGACAAAGCTGAATGATGATTTGTTTTTTATTTATTTTTA | 651 |
| 66944119 | 66819683 | AATCCAAACTTacaattttacatgtcattg | |
| chrX: 66763874- | chrX: 66821160- | AAAGTACATTTTGGCCCATTTACTCCTTAAATAATTTTATGTCTCCCAAGGAGAGTTGTAAGTTGCTTGATAGTAAATGCTATGTATTTT | 652 |
| 66944119 | 66821279 | GTACCTTAGTGTATATattatgggatttca | |
| chrX: 66763874- | chrX: 66820740- | TTATAAACAAAATAAAGGTAATTTGGAGGAAAACTGTCTTCAGTTATATTGGATATTTGGGGGACATTTTTGTATGTTAGTTAGCAAAGA | 653 |
| 66944119 | 66820859 | TCACTTGAAAAAGAAGATTCTTCCTTCTAT | |
| chrX: 66763874- | chrX: 66821040- | TTAAAATAAATTAAATGGATGATTTCAGCCAGATCATTATGAAAACACATGAGATATTGGGTTATGCAATGACTAACAGTGTGTACCTTT | 654 |
| 66944119 | 66821159 | TCTTGATATTTATTCATAAACTGGGGAATA | |
| chrX: 66763874- | chrX: 66820680- | tcacaaaattaaaaAAAACTGTGCATTAAAGAAAAACAAAAATAAAAACCATAGTTCAAGTTATAAACAAAATAAAGGTAATTTGGAGGA | 655 |
| 66944119 | 66820799 | AAACTGTCTTCAGTTATATTGGATATTTGG | |
| chrX: 66763874- | chrX: 66821100- | GTTATGCAATGACTAACAGTGTGTACCTTTTCTTGATATTTATTCATAAACTGGGGAATAAAAGTACATTTTGGCCCATTTACTCCTTAA | 656 |
| 66944119 | 66821219 | ATAATTTTATGTCTCCCAAGGAGAGTTGTA | |
| chrX: 66763874- | chrX: 66820860- | GATTCAAGGGAGCCTAGCAAAAAATAAATGAAATGAAATAAAATAATACAAAGAGAAAAGATTATTCCATAAATTCTGCTTACTTATTTC | 657 |
| 66944119 | 66820979 | TGGCAAACTTGTTGACAGCACATGTGACCT | |
| chrX: 66763874- | chrX: 66820920- | ATTATTCCATAAATTCTGCTTACTTATTTCTGGCAAACTTGTTGACAGCACATGTGACCTTTTGGTAAAAAGACATTTTTATATTTTTAG | 658 |
| 66944119 | 66821039 | TTAAGTTTCAAATATAAATTGTTTGTGTTT | |
| chrX: 66763874- | chrX: 66820980- | TTTGGTAAAAAGACATTTTTATATTTTTAGTTAAGTTTCAAATATAAATTGTTTGTGTTTTTAAAATAAATTAAATGGATGATTTCAGCC | 659 |
| 66944119 | 66821099 | AGATCATTATGAAAACACATGAGATATTGG | |
| chrX: 66763874- | chrX: 66820800- | GGGACATTTTTGTATGTTAGTTAGCAAAGATCACTTGAAAAAGAAGATTCTTCCTTCTATGATTCAAGGGAGCCTAGCAAAAAATAAATG | 660 |
| 66944119 | 66820919 | AAATGAAATAAAATAATACAAAGAGAAAAG | |
| chrX: 66763874- | chrX: 66821375- | ACACTCAATAATGTAGTTAGTGAATTATTTAGAAAGAGGCATTTTGAGCCCATAATGTATGATAGGTACTTCTACATTTATTATTTTATT | 661 |
| 66944119 | 66821494 | CTTTGCAGACCTGCAGAAAACTGTAAGAAA | |
| chrX: 66763874- | chrX: 66821435- | GATAGGTACTTCTACATTTATTATTTTATTCTTTGCAGACCTGCAGAAAACTGTAAGAAAAAAGTTTATTTCAGATTCATGTGTTTATTT | 662 |
| 66944119 | 66821554 | GATTAATCTCTTCATAGGTTTCATTTTTCA | |
| chrX: 66763874- | chrX: 66821495- | AAAGTTTATTTCAGATTCATGTGTTTATTTGATTAATCTCTTCATAGGTTTCATTTTTCAGCTCCTGTCAGAAAATACAGATTCTTATAA | 663 |
| 66944119 | 66821614 | GGTTCACCTTTTACCCATAAGAATAATAGT | |
| chrX: 66763874- | chrX: 66821980- | TAGAAAGGATAATTCTTATTTCATATTTATCTTTTTGTTTCAGAATAATAAACTAAGCTATCTCTACTCAGTCCATTTTAATACAAAAAT | 664 |
| 66944119 | 66822099 | ATTTTTACCCGGACTGAGTTTTTATGCTTT | |
| chrX: 66763874- | chrX: 66821920- | ACCATAACCTCTTTATAAAAATTTTCTGGAAAGTTTACATGACAGTAAGTAATATATAATTAGAAAGGATAATTCTTATTTCATATTTAT | 665 |
| 66944119 | 66822039 | CTTTTTGTTTCAGAATAATAAACTAAGCTA | |
| chrX: 66763874- | chrX: 66821860- | ATAGCTTGCCCACAGTAATGTGTCCTGAAAAATATGACAATTAATTAAGTTGGAGACAGAACCATAACCTCTTTATAAAAATTTTCTGGA | 666 |
| 66944119 | 66821979 | AAGTTTACATGACAGTAAGTAATATATAAT | |
| chrX: 66763874- | chrX: 66821800- | ccaTTTTAGTGAGATTCCTGTGCTGATAGTCATACCCATATCAAATCCTCTTTGGCAGTTATAGCTTGCCCACAGTAATGTGTCCTGAAA | 667 |
| 66944119 | 66821919 | AATATGACAATTAATTAAGTTGGAGACAGA | |
| chrX: 66763874- | chrX: 66822040- | TCTCTACTCAGTCCATTTTAATACAAAAATATTTTTACCCGGACTGAGTTTTTATGCTTTTTAGGAACTTTGTATCTGCCTCACTTAGTT | 668 |
| 66944119 | 66822159 | AAAATCCTAGCTGCACTAATCACTTACtgt | |
| chrX: 66763874- | chrX: 66822955- | AAATTGTCTCTAGGTAGACTTTCCCACAATGCAATTTTAGGATACAGAGGTCATATGCCTGTTATTCTACTGTGGCAGAGAAAATATGGA | 669 |
| 66944119 | 66823074 | GCCTGGAAAACTGttcatttgcatcacata | |
| chrX: 66763874- | chrX: 66822895- | TACTGATTCTCCCAGGCCTATACAAATCCTTTGATACACAAATGAATAGTAAAGGAACATAAATTGTCTCTAGGTAGACTTTCCCACAAT | 670 |
| 66944119 | 66823014 | GCAATTTTAGGATACAGAGGTCATATGCCT | |
| chrX: 66763874- | chrX: 66822715- | caataaaaaactaatataTTTACCATGCAAGGCAAGGCATTTATCCTCTCATGATTCAGTTTCTTTTTACCTGACATAATGGAATTAATT | 671 |
| 66944119 | 66822834 | TATACTGCTGTGAAGTTGTAGTTGAGAAAC | |
| chrX: 66763874- | chrX: 66822835- | ATGACTTCTAAAGTAATAGAGGACATGTATTATTAATTTTAGTAGTATTAATAGTAATGATACTGATTCTCCCAGGCCTATACAAATCCT | 672 |
| 66944119 | 66822954 | TTGATACACAAATGAATAGTAAAGGAACAT | |
| chrX: 66763874- | chrX: 66822775- | TTCTTTTTACCTGACATAATGGAATTAATTTATACTGCTGTGAAGTTGTAGTTGAGAAACATGACTTCTAAAGTAATAGAGGACATGTAT | 673 |
| 66944119 | 66822894 | TATTAATTTTAGTAGTATTAATAGTAATGA | |
| chrX: 66763874- | chrX: 66823692- | AGAGATGATTTAGAAGAGTTGTTTTGGAAAAGGAGAAGACAGAAAATGTTTTAGAGGTGTCATAGAGATAAAATTGGCATGGCATGGTGC | 674 |
| 66944119 | 66823811 | AAGGAGGTAAAGCCCAATAGCTTTGTAAGG | |
| chrX: 66763874- | chrX: 66823916- | TGAGAGGGTGGGTAACAGCAATAGTCAGAGGAAAGAACCCTTTTATACATGATGGTACAGGAACAACACTGGCTTCCAACCCCACAGCTG | 675 |
| 66944119 | 66824035 | CTCTTTAACAGAAGGTCAGAAGCTGGGGAG | |
| chrX: 66763874- | chrX: 66824165- | TCACCCGTTCCAAGAGGCCTTAAAGGTGTTGATCTGTTCCCTGGGCATCACCACATTCCACAAATTAATGTTCCTCTGAGAGAATAGGGT | 676 |
| 66944119 | 66824284 | GATTCAATTTCACTGTGCCCGAAGGTTACT | |
| chrX: 66763874- | chrX: 66824345- | TTTCTCTAACCCTTAGCATGTATAAACTGATCTGTTGGGAAATGTGTAGCATTTATAGGATGGTAGGATTTGTAACATGCGATCACAGGA | 677 |
| 66944119 | 66824464 | CTGTTTATATAGAGTCCCTGGGAAGGGGAG | |
| chrX: 66763874- | chrX: 66824285- | TTTGGGGTTCATGTTTGTTCTAAGTCTATGCTAATGATCTGCCAACTGTCTGTTTGTCACTTTCTCTAACCCTTAGCATGTATAAACTGA | 678 |
| 66944119 | 66824404 | TCTGTTGGGAAATGTGTAGCATTTATAGGA | |
| chrX: 66763874- | chrX: 66824225- | CAAATTAATGTTCCTCTGAGAGAATAGGGTGATTCAATTTCACTGTGCCCGAAGGTTACTTTTGGGGTTCATGTTTGTTCTAAGTCTATG | 679 |
| 66944119 | 66824344 | CTAATGATCTGCCAACTGTCTGTTTGTCAC | |
| chrX: 66763874- | chrX: 66824105- | CTAAGGATGGGAAGTAGATTAGTTGAGGCCACTGCAGTGGGGTCTGCAAGTTGCTAGCACTCACCCGTTCCAAGAGGCCTTAAAGGTGTT | 680 |
| 66944119 | 66824224 | GATCTGTTCCCTGGGCATCACCACATTCCA | |
| chrX: 66763874- | chrX: 66825267- | ACAGAGGACACAGTTGGCATTTCCTTTTGGTGTTGAGGGGAGATGTGTACATGGTTGTGAGATGACTCACCCTTTTTGCTTAGATAGTTC | 681 |
| 66944119 | 66825386 | CACTTTCATTGTGGACAGACTCTTTGGAGG | |
| chrX: 66763874- | chrX: 66824607- | TCAAATAAATTAGTCATTGGTGCCAGAGTATCAAATAATTATGGTACAGAATGTATTTCTCTGAATGACACCTTCTCCCAGAGATTCTGA | 682 |
| 66944119 | 66824726 | TATATATTCCTCTGCACTCACCCTGTTTGA | |
| chrX: 66763874- | chrX: 66825087- | CTCAGGGACCAGTGTGAGAATGGGAACTTTATGATCTGGAGCTGGTTAAGTGAAGTCCAAAAATAATTAAGAAAGTGTTTCCTTCCCTGG | 683 |
| 66944119 | 66825206 | GAATGAGTTCAGTAGGAATCTCAATGTATT | |
| chrX: 66763874- | chrX: 66825147- | AAATAATTAAGAAAGTGTTTCCTTCCCTGGGAATGAGTTCAGTAGGAATCTCAATGTATTGTAGAGCACTAAGGACTCAGCCTCAGGCAT | 684 |
| 66944119 | 66825266 | TTGCAAAGGATTCTTCCAGTTGCCTGTGTT | |
| chrX: 66763874- | chrX: 66825447- | ATTTGTTGAGTGGTTTGCCATTTTGTTTTACAGCCACTCTGTGGGCTATGAAATGGTCATCCGGCCGCTTTATTTGTCCCTAAAAAAAGC | 685 |
| 66944119 | 66825566 | AGTTTTTCCCTTTCTTATCTTCATGGCTGC | |
| chrX: 66763874- | chrX: 66824667- | CTGAATGACACCTTCTCCCAGAGATTCTGATATATATTCCTCTGCACTCACCCTGTTTGATAATTACCAGTATATGGACCATTTACCTGA | 686 |
| 66944119 | 66824786 | AGAATAAGAGTAGGGTTTCCTACTGTTGTT | |
| chrX: 66763874- | chrX: 66824547- | GCACATATCCATTTTCAATAAACATGAAAGTTTCATACCCTCTTTTAATGTTTGAAATCCTCAAATAAATTAGTCATTGGTGCCAGAGTA | 687 |
| 66944119 | 66824666 | TCAAATAATTATGGTACAGAATGTATTTCT | |
| chrX: 66763874- | chrX: 66825207- | GTAGAGCACTAAGGACTCAGCCTCAGGCATTTGCAAAGGATTCTTCCAGTTGCCTGTGTTACAGAGGACACAGTTGGCATTTCCTTTTGG | 688 |
| 66944119 | 66825326 | TGTTGAGGGGAGATGTGTACATGGTTGTGA | |
| chrX: 66763874- | chrX: 66825387- | GCCAGTTTGGCATGCACGTGTGTGTTCATTCCATCCTGGAGCATTCTTTATGAGAAAGCCATTTGTTGAGTGGTTTGCCATTTTGTTTTA | 689 |
| 66944119 | 66825506 | CAGCCACTCTGTGGGCTATGAAATGGTCAT | |
| chrX: 66763874- | chrX: 66824727- | TAATTACCAGTATATGGACCATTTACCTGAAGAATAAGAGTAGGGTTTCCTACTGTTGTTGAAAATTTGCTTGACTCTTAACAACTTGTG | 690 |
| 66944119 | 66824846 | TGTGACTGTAACAAGATCACACAGGGTAAA | |
| chrX: 66763874- | chrX: 66824787- | GAAAATTTGCTTGACTCTTAACAACTTGTGTGTGACTGTAACAAGATCACACAGGGTAAACAATATTAGCTTATTCAACCACTGGCTGAA | 691 |
| 66944119 | 66824906 | GAAATTTAGGAAAGTGAACACATTTTTCTT | |
| chrX: 66763874- | chrX: 66824847- | CAATATTAGCTTATTCAACCACTGGCTGAAGAAATTTAGGAAAGTGAACACATTTTTCTTTACATTTCTCTTTGTTCTGTGAGCCTTTTA | 692 |
| 66944119 | 66824966 | TGCTGGAATAGTTTTCACTGCAGGCTGTTA | |
| chrX: 66763874- | chrX: 66825627- | AAACTGATGATGTATTGGATTTGATAATTCATCAAATCTGAGGTTTACTGGTTTGTATTTGCCTCAAAATGGGCATATAATATTTTGTCA | 693 |
| 66944119 | 66825746 | GGTAACATAATAGACAGATCATTGGCATTG | |
| chrX: 66763874- | chrX: 66825507- | CCGGCCGCTTTATTTGTCCCTAAAAAAAGCAGTTTTTCCCTTTCTTATCTTCATGGCTGCCAAGCAGCAGAAAGAGTAACTCAGGGAAGC | 694 |
| 66944119 | 66825626 | CATGTGATAGCCTTTTATCTGTCTGTTCAG | |
| chrX: 66763874- | chrX: 66825327- | GATGACTCACCCTTTTTGCTTAGATAGTTCCACTTTCATTGTGGACAGACTCTTTGGAGGGCCAGTTTGGCATGCACGTGTGTGTTCATT | 695 |
| 66944119 | 66825446 | CCATCCTGGAGCATTCTTTATGAGAAAGCC | |
| chrX: 66763874- | chrX: 66825567- | CAAGCAGCAGAAAGAGTAACTCAGGGAAGCCATGTGATAGCCTTTTATCTGTCTGTTCAGAAACTGATGATGTATTGGATTTGATAATTC | 696 |
| 66944119 | 66825686 | ATCAAATCTGAGGTTTACTGGTTTGTATTT | |
| chrX: 66763874- | chrX: 66825027- | ACCTCTTTCCAAGGTTAGTTGCCAATGGCATCTTTGGAACAGTGTCCTTCACTTTTGTCCCTCAGGGACCAGTGTGAGAATGGGAACTTT | 697 |
| 66944119 | 66825146 | ATGATCTGGAGCTGGTTAAGTGAAGTCCAA | |
| chrX: 66763874- | chrX: 66824967- | TTGTCTGCCTCCAGAGGAGGGAGTTGACCTAGCAGTGGTAACTGGAGAGTGTTTTTTGAAACCTCTTTCCAAGGTTAGTTGCCAATGGCA | 698 |
| 66944119 | 66825086 | TCTTTGGAACAGTGTCCTTCACTTTTGTCC | |
| chrX: 66763874- | chrX: 66824907- | TACATTTCTCTTTGTTCTGTGAGCCTTTTATGCTGGAATAGTTTTCACTGCAGGCTGTTATTGTCTGCCTCCAGAGGAGGGAGTTGACCT | 699 |
| 66944119 | 66825026 | AGCAGTGGTAACTGGAGAGTGTTTTTTGAA | |
| chrX: 66763874- | chrX: 66825885- | agcaaaTTGCTATTTTTTTTAAATTTCATAGTGTACATGAGTATAAGGTGCTGAATATGTGATTGATTCTGAGGGAAAAGAGAGATAAGG | 700 |
| 66944119 | 66826004 | GAAAGTTCTCAGAGAAAGTCAAGCTgaggg | |
| chrX: 66763874- | chrX: 66826641- | AAAATACAGAGAAGTGAAACCAGAGATAGTGGGATCATTCTGGAGTCTGTTGCCTACACTGAACAGTAGTTGAGCGAAAAAGGATGGGCA | 701 |
| 66944119 | 66826760 | GAATGTGTTGGTTCTGGGTATTGCAAATTC | |
| chrX: 66763874- | chrX: 66826461- | ATAATTTGCTATTCACCAAGTACTTCTTACGTACACTGTGCATGAAATGATTATTACTTTTTCTAATATTAGTTTTCTTGATTGAGGCTT | 702 |
| 66944119 | 66826580 | GGCAATTATTAGTTTGTATGCCTTTAGAAG | |
| chrX: 66763874- | chrX: 66826761- | ATGGCACTTGAGTGAAAAAGTTTAAGCCTTCTATTGGCTCTTTGTGAATATCTTCAACATGCATGACTACAAATAGAACACATGGTTTTG | 703 |
| 66944119 | 66826880 | TTGTTATTGTTGTTGTGtttttgttttttt | |
| chrX: 66763874- | chrX: 66826581- | GATCATAAGCAGAGGTTTATCCCAGTAGGATTTGCATTTTAGAATGATGACTTTGGGAGTAAAATACAGAGAAGTGAAACCAGAGATAGT | 704 |
| 66944119 | 66826700 | GGGATCATTCTGGAGTCTGTTGCCTACACT | |
| chrX: 66763874- | chrX: 66826401- | tttttaccctttgaAGATATTGAACTGTTTTATGAACACAATCTTAGAAGGATTTAAAAAATAATTTGCTATTCACCAAGTACTTCTTAC | 705 |
| 66944119 | 66826520 | GTACACTGTGCATGAAATGATTATTACTTT | |
| chrX: 66763874- | chrX: 66826701- | GAACAGTAGTTGAGCGAAAAAGGATGGGCAGAATGTGTTGGTTCTGGGTATTGCAAATTCATGGCACTTGAGTGAAAAAGTTTAAGCCTT | 706 |
| 66944119 | 66826820 | CTATTGGCTCTTTGTGAATATCTTCAACAT | |
| chrX: 66763874- | chrX: 66826521- | TTCTAATATTAGTTTTCTTGATTGAGGCTTGGCAATTATTAGTTTGTATGCCTTTAGAAGGATCATAAGCAGAGGTTTATCCCAGTAGGA | 707 |
| 66944119 | 66826640 | TTTGCATTTTAGAATGATGACTTTGGGAGT | |
| chrX: 66763874- | chrX: 66827757- | CCAACTTTGCGCTCACATCTAGTTCTGTTGCCCATGTGCAAGCTGAATTTGGGCCCGGGCCCCCAGATCTAACATGAAACTCAAGTTTCC | 708 |
| 66944119 | 66827876 | TTCTGTTCAAACTGTCCAGGCATAATAGTC | |
| chrX: 66763874- | chrX: 66827877- | TTAAAGTCCGATGCCCAGCAGAGCCGTAGATTTTTCACTGGCCAAAAATCAACATGAAACCAGATGTATCTGTAAATCTAGTTTCATAAC | 709 |
| 66944119 | 66827996 | ACTTTGTAGTCAATGGAAATACAGTAGCAG | |
| chrX: 66763874- | chrX: 66827517- | ttaagaattagtaAAAATAGTAAAAGAACAATTCATTCTCCATCCAGATGTTCTGTCCCCACTGTGACTTATGTGCTCATTCAGAGTTGT | 710 |
| 66944119 | 66827636 | ACAGAAAAACCTCCACTTAATTTTCACAAG | |
| chrX: 66763874- | chrX: 66827817- | CCCCAGATCTAACATGAAACTCAAGTTTCCTTCTGTTCAAACTGTCCAGGCATAATAGTCTTAAAGTCCGATGCCCAGCAGAGCCGTAGA | 711 |
| 66944119 | 66827936 | TTTTTCACTGGCCAAAAATCAACATGAAAC | |
| chrX: 66763874- | chrX: 66827937- | CAGATGTATCTGTAAATCTAGTTTCATAACACTTTGTAGTCAATGGAAATACAGTAGCAGGCAGACCAGACCAGAGTTTACTATTTGCAG | 712 |
| 66944119 | 66828056 | TGGAATTAATAACCACATGGAAACTTTGCC | |
| chrX: 66763874- | chrX: 66828057- | TTTGGTATCTGCGAGATGGAAGATAAAGGTGCGAATTCAAAGCAGTTCCCACCTTACCCTCTAAATTCCAACATAAAGAGGCCTTGAATG | 713 |
| 66944119 | 66828176 | TCCTTCTATCTTATTGTATATTTCATTAAC | |
| chrX: 66763874- | chrX: 66828477- | GAAGTGTTTTTAAAAGATTTAAAATCGAAATAATATAAAAGAATGTTAAAAACAAGTAAAACATATCACTAGTTAATCACTCTACCAAAA | 714 |
| 66944119 | 66828596 | TTCATTTTTATGTTTGCATATTTAACCATT | |
| chrX: 66763874- | chrX: 66828117- | CTAAATTCCAACATAAAGAGGCCTTGAATGTCCTTCTATCTTATTGTATATTTCATTAACAGAAGTATGTTCCTAGCTACTTAGTCATTC | 715 |
| 66944119 | 66828236 | TATCTCTATTCTCCTTTGTTTTAACTTCAG | |
| chrX: 66763874- | chrX: 66827637- | CTGGAGTTCCACATGTAACAGAATCATATGGGACCAAAAAATTCTCTGTATTGGCTTCTTCCCTGCCGTATTTTGGCTCTGGGACCAACA | 716 |
| 66944119 | 66827756 | AGACACCCATTTTGCATGAGCTGCCTGCCA | |
| chrX: 66763874- | chrX: 66828357- | AGGAAATTTAGAATTGCCTAAAGAAAGGATGTATTGGCCAACCTAATAATAAATCAGTATTAGTGAATCTAAAGCATATTTGAAAAATTT | 717 |
| 66944119 | 66828476 | GTAACATGAGTTGAAATTCAGACCTGCAAT | |
| chrX: 66763874- | chrX: 66828537- | ACATATCACTAGTTAATCACTCTACCAAAATTCATTTTTATGTTTGCATATTTAACCATTTTTATTTTCTATATTTGTCCATGAACATGT | 718 |
| 66944119 | 66828656 | GTTTTTATATATTGTTTATATTAAACATGG | |
| chrX: 66763874- | chrX: 66828297- | TTATCTTTAGGGACAGAAATGTTAGGAAGATCCTAGTTCCTCATCTCTTTGCTCCTGACAAGGAAATTTAGAATTGCCTAAAGAAAGGAT | 719 |
| 66944119 | 66828416 | GTATTGGCCAACCTAATAATAAATCAGTAT | |
| chrX: 66763874- | chrX: 66828237- | TGGTGCCAGCTTAAGATGCTCTGGCTTTCAGCTTTCATGGAGCACGTCATGTTTTTAAACTTATCTTTAGGGACAGAAATGTTAGGAAGA | 720 |
| 66944119 | 66828356 | TCCTAGTTCCTCATCTCTTTGCTCCTGACA | |
| chrX: 66763874- | chrX: 66828177- | AGAAGTATGTTCCTAGCTACTTAGTCATTCTATCTCTATTCTCCTTTGTTTTAACTTCAGTGGTGCCAGCTTAAGATGCTCTGGCTTTCA | 721 |
| 66944119 | 66828296 | GCTTTCATGGAGCACGTCATGTTTTTAAAC | |
| chrX: 66763874- | chrX: 66827997- | GCAGACCAGACCAGAGTTTACTATTTGCAGTGGAATTAATAACCACATGGAAACTTTGCCTTTGGTATCTGCGAGATGGAAGATAAAGGT | 722 |
| 66944119 | 66828116 | GCGAATTCAAAGCAGTTCCCACCTTACCCT | |
| chrX: 66763874- | chrX: 66828597- | TTTATTTTCTATATTTGTCCATGAACATGTGTTTTTATATATTGTTTATATTAAACATGGTTTTAATCATGGCTTATTTCTTTTATGTTT | 723 |
| 66944119 | 66828716 | TACTTCTTTTCCTTTGACataaaatattgt | |
| chrX: 66763874- | chrX: 66828417- | TAGTGAATCTAAAGCATATTTGAAAAATTTGTAACATGAGTTGAAATTCAGACCTGCAATGAAGTGTTTTTAAAAGATTTAAAATCGAAA | 724 |
| 66944119 | 66828536 | TAATATAAAAGAATGTTAAAAACAAGTAAA | |
| chrX: 66763874- | chrX: 66827577- | ACTGTGACTTATGTGCTCATTCAGAGTTGTACAGAAAAACCTCCACTTAATTTTCACAAGCTGGAGTTCCACATGTAACAGAATCATATG | 725 |
| 66944119 | 66827696 | GGACCAAAAAATTCTCTGTATTGGCTTCTT | |
| chrX: 66763874- | chrX: 66827697- | CCCTGCCGTATTTTGGCTCTGGGACCAACAAGACACCCATTTTGCATGAGCTGCCTGCCACCAACTTTGCGCTCACATCTAGTTCTGTTG | 726 |
| 66944119 | 66827816 | CCCATGTGCAAGCTGAATTTGGGCCCGGGC | |
| chrX: 66763874- | chrX: 66829406- | TTCTCTAAAAATTATTCTAAGAAAAGACAAAGGTGATACGAAATATATCCTGAGTTTTTATTTTTTTCTTGCATGGGATTTGTATATTTG | 727 |
| 66944119 | 66829525 | CACCTTTGCCCATTTATACTATGATTTCTT | |
| chrX: 66763874- | chrX: 66829886- | AAGGCCCTTAAACATTTTTAGGCTTAATTGGCTGTCCTTGTACTTAGGGCACATCTAAAAATCCTGAGGCAACCACTCAAGAGAACATGC | 728 |
| 66944119 | 66830005 | TTTTGTTAATTCAAAGGGAGCTGTCCTACG | |
| chrX: 66763874- | chrX: 66829466- | TTTTTTTCTTGCATGGGATTTGTATATTTGCACCTTTGCCCATTTATACTATGATTTCTTAGTGTCTTCCCTGGCAATTTTAATGAAGAC | 729 |
| 66944119 | 66829585 | TTCATGTATATCAATTTTTCCACAAATATA | |
| chrX: 66763874- | chrX: 66830006- | AGTGTCCAGAATCCTCTGTAGTCTTGGGCCTGGTGCTTGAGAGACCCAAAGGAAAGGTCAATGGAATTACAGCTTAGTGTTAGAGCTTTC | 730 |
| 66944119 | 66830125 | ATGCATCACACTAATTAATTAATGTCATAA | |
| chrX: 66763874- | chrX: 66830066- | ATGGAATTACAGCTTAGTGTTAGAGCTTTCATGCATCACACTAATTAATTAATGTCATAAAGGTCTCTCTCCTGTTATGGGAAAAAGCAG | 731 |
| 66944119 | 66830185 | CAAATAGGAACTTCTGGTAGGGTGCTTAAA | |
| chrX: 66763874- | chrX: 66829526- | AGTGTCTTCCCTGGCAATTTTAATGAAGACTTCATGTATATCAATTTTTCCACAAATATAATCTTTCTAAAAATATGTTTTTTCCACAAT | 732 |
| 66944119 | 66829645 | ATAATTCAGACGTATTCTCCGAAATGTTGG | |
| chrX: 66763874- | chrX: 66830126- | AGGTCTCTCTCCTGTTATGGGAAAAAGCAGCAAATAGGAACTTCTGGTAGGGTGCTTAAAGTTGGTTTGATATTTTTTATTAGCATTTTT | 733 |
| 66944119 | 66830245 | AACTAATACAAGTAATACATGCTTATGGTA | |
| chrX: 66763874- | chrX: 66829826- | GAGGTAGGGATTATTTTGTAACTGCCCCCAACCTTCTAACCTGTAATGAAACAAACACTGAAGGCCCTTAAACATTTTTAGGCTTAATTG | 734 |
| 66944119 | 66829945 | GCTGTCCTTGTACTTAGGGCACATCTAAAA | |
| chrX: 66763874- | chrX: 66829766- | GGGCAAGACCACTTAACTCTTGGAAAATACAGGAAACGTAGATTTCTAGAGGCCAAGAAGGAGGTAGGGATTATTTTGTAACTGCCCCCA | 735 |
| 66944119 | 66829885 | ACCTTCTAACCTGTAATGAAACAAACACTG | |
| chrX: 66763874- | chrX: 66829586- | ATCTTTCTAAAAATATGTTTTTTCCACAATATAATTCAGACGTATTCTCCGAAATGTTGGAAAAACTTAAGTAGGCATCAAAGCATTTGA | 736 |
| 66944119 | 66829705 | AGATTTGTTTAAAGGTTGTTTTTATACCAG | |
| chrX: 66763874- | chrX: 66829646- | AAAAACTTAAGTAGGCATCAAAGCATTTGAAGATTTGTTTAAAGGTTGTTTTTATACCAGTTTTAAATTGTAATTTAAGGGTCATAAAAT | 737 |
| 66944119 | 66829765 | AGGTGAAAATTAAATCATTTTTCAGTAAGG | |
| chrX: 66763874- | chrX: 66830186- | GTTGGTTTGATATTTTTTATTAGCATTTTTAACTAATACAAGTAATACATGCTTATGGTAGAATGATAAAACTGAAAAAAAAGGTATGAA | 738 |
| 66944119 | 66830305 | AATTTAGAAGTTCTCCTACTCATGACCTCA | |
| chrX: 66763874- | chrX: 66829706- | TTTTAAATTGTAATTTAAGGGTCATAAAATAGGTGAAAATTAAATCATTTTTCAGTAAGGGGGCAAGACCACTTAACTCTTGGAAAATAC | 739 |
| 66944119 | 66829825 | AGGAAACGTAGATTTCTAGAGGCCAAGAAG | |
| chrX: 66763874- | chrX: 66829946- | ATCCTGAGGCAACCACTCAAGAGAACATGCTTTTGTTAATTCAAAGGGAGCTGTCCTACGAGTGTCCAGAATCCTCTGTAGTCTTGGGCC | 740 |
| 66944119 | 66830065 | TGGTGCTTGAGAGACCCAAAGGAAAGGTCA | |
| chrX: 66763874- | chrX: 66829346- | TTCCTTTTTTCTAGATTTTATTTTTATTTAAGGCATCCTTTGATTTTAACCTGATTTTTTTTCTCTAAAAATTATTCTAAGAAAAGACAA | 741 |
| 66944119 | 66829465 | AGGTGATACGAAATATATCCTGAGTTTTTA | |
| chrX: 66763874- | chrX: 66830911- | TATATAATTGTTAGTATAATTGTATACCTTTATTATCATACTTTTGCATGTATAGCAATAAGACAAATTCTTAGATGTTTAACCATTGGA | 742 |
| 66944119 | 66831030 | CATAAGGAATGTACGCATTTTAAGTACTGG | |
| chrX: 66763874- | chrX: 66831798- | AATTTTTTTTTTATTTTTCAGATTAATTTTCACTTAGAGATTCATCAGCATATGTACTATACATGTACAAATCACCTGTGTGTTTTGGAT | 743 |
| 66944119 | 66831917 | ATTTAGTTAAACAAATGTGCAAATATTTTA | |
| chrX: 66763874- | chrX: 66831738- | ATGGTTTCACAAAAAGAGCTTTCTACTAAAAAATAAAATACATACTTAAGCAACTCAGAGAATTTTTTTTTTATTTTTCAGATTAATTTT | 744 |
| 66944119 | 66831857 | CACTTAGAGATTCATCAGCATATGTACTAT | |
| chrX: 66763874- | chrX: 66832338- | GGGCAACTTTTTTCTTGCCTGTCTGCCTGCCTAAGCCAACTTAAATAAACATCATTTCTGTAACTTCTGTGAAGCCTTTTCCAATCTCTC | 745 |
| 66944119 | 66832457 | CACTCCAAGACGAAGGTGTTTCTATAGGCA | |
| chrX: 66763874- | chrX: 66831918- | ACCAAAGGAGCATATTCATTTGTGTTTTATTTTCTTAATGGTTTTCGTTATGAATGTGAAATGTGTATTTACCTTAACAGAAATTAAGTA | 746 |
| 66944119 | 66832037 | TATTTTTGGTCTGACATATATGAGAACTGA | |
| chrX: 66763874- | chrX: 66832278- | ACCTATGCAGTCTTCCAGTCTTATTTACACATTCCTTTGCACATGCTGTTTCCCCGTGTGGGGCAACTTTTTTCTTGCCTGTCTGCCTGC | 747 |
| 66944119 | 66832397 | CTAAGCCAACTTAAATAAACATCATTTCTG | |
| chrX: 66763874- | chrX: 66832038- | AAAGCATTGGCTTGGCTGCTAACTGCATTCTCATCTTTCTTTCTCTGCTTTGGCAAAGTCTGGGATTAAATCTAATACCTTTTAAACTGT | 748 |
| 66944119 | 66832157 | TTGGGACTTCAGCCAGAGTGACCTGTCTTG | |
| chrX: 66763874- | chrX: 66831858- | ACATGTACAAATCACCTGTGTGTTTTGGATATTTAGTTAAACAAATGTGCAAATATTTTAACCAAAGGAGCATATTCATTTGTGTTTTAT | 749 |
| 66944119 | 66831977 | TTTCTTAATGGTTTTCGTTATGAATGTGAA | |
| chrX: 66763874- | chrX: 66832158- | AATTCAGAACTGCGCAGATCATTCCCCATTCTAAGGCCCTCTCATGCCTCCTCATTGCCTGTAGGATGAGATCCAAGTACCTTAGCATAG | 750 |
| 66944119 | 66832277 | CTTATGCACTGTAGTCACTTGACCTCTAGC | |
| chrX: 66763874- | chrX: 66831978- | ATGTGTATTTACCTTAACAGAAATTAAGTATATTTTTGGTCTGACATATATGAGAACTGAAAAGCATTGGCTTGGCTGCTAACTGCATTC | 751 |
| 66944119 | 66832097 | TCATCTTTCTTTCTCTGCTTTGGCAAAGTC | |
| chrX: 66763874- | chrX: 66832218- | GTAGGATGAGATCCAAGTACCTTAGCATAGCTTATGCACTGTAGTCACTTGACCTCTAGCACCTATGCAGTCTTCCAGTCTTATTTACAC | 752 |
| 66944119 | 66832337 | ATTCCTTTGCACATGCTGTTTCCCCGTGTG | |
| chrX: 66763874- | chrX: 66832098- | TGGGATTAAATCTAATACCTTTTAAACTGTTTGGGACTTCAGCCAGAGTGACCTGTCTTGAATTCAGAACTGCGCAGATCATTCCCCATT | 753 |
| 66944119 | 66832217 | CTAAGGCCCTCTCATGCCTCCTCATTGCCT | |
| chrX: 66763874- | chrX: 66833446- | CCCTCTCAAAAGCAACCTAAACAGGATGTGGGGTAGGCTCTAAAGCATTCCTCAAGCCACACATGGATCCATCAGTAAAATGTGGAGGGC | 754 |
| 66944119 | 66833565 | TTAAGGATAAAAAGGCTTAAGTACAATCTC | |
| chrX: 66763874- | chrX: 66833386- | cttgtggAATTGCACTCACTGCTGGGTGTGTGGAAAGTTATGCATGTGTGCTTTACTGTACCCTCTCAAAAGCAACCTAAACAGGATGTG | 755 |
| 66944119 | 66833505 | GGGTAGGCTCTAAAGCATTCCTCAAGCCAC | |
| chrX: 66763874- | chrX: 66833926- | CAGAAGGCTTAAAGTCAGGTTAGGGAAAGGAGGCCTATGAGGTTACTGTGCAGAGGCAGTGCTGGGAAATAAATGAAGTTAAATAAATTT | 756 |
| 66944119 | 66834045 | AGGCCATCGTGGTTTAAAGAATGGATTGTG | |
| chrX: 66763874- | chrX: 66833626- | GGCAAAATAATAAAATCATATTTGTCTCTAGTGGAATGGATAACTATGCCTAAAACTGTGCCCTTTGAAAAGCAACTAGAGAGATAATTT | 757 |
| 66944119 | 66833745 | CTGAAGTGTTTGTCCCTACCTGAATGTGTG | |
| chrX: 66763874- | chrX: 66833566- | TGGCCCTACATTTTCTAAATGTTATGCCACCCTGACCAAGGGGCAACTCCTACAAAGCCAGGCAAAATAATAAAATCATATTTGTCTCTA | 758 |
| 66944119 | 66833685 | GTGGAATGGATAACTATGCCTAAAACTGTG | |
| chrX: 66763874- | chrX: 66833686- | CCCTTTGAAAAGCAACTAGAGAGATAATTTCTGAAGTGTTTGTCCCTACCTGAATGTGTGGCAAAATTCTAAACTCCCTGAAGTGTGAAA | 759 |
| 66944119 | 66833805 | GTGGTTTCCAAGCCACATGCACATCCAGTA | |
| chrX: 66763874- | chrX: 66833746- | GCAAAATTCTAAACTCCCTGAAGTGTGAAAGTGGTTTCCAAGCCACATGCACATCCAGTAGTGGTAAAGGGTGAAAATCTAACTGGCTAA | 760 |
| 66944119 | 66833865 | GAGGGCTTCATAGCAACATTAACCAAAAAG | |
| chrX: 66763874- | chrX: 66833866- | TGGTTTATGTAGTCTTTGCCTGCTTCATAATTCCCTAGGCATTCTATGCTATTCTGTACTCAGAAGGCTTAAAGTCAGGTTAGGGAAAGG | 761 |
| 66944119 | 66833985 | AGGCCTATGAGGTTACTGTGCAGAGGCAGT | |
| chrX: 66763874- | chrX: 66833806- | GTGGTAAAGGGTGAAAATCTAACTGGCTAAGAGGGCTTCATAGCAACATTAACCAAAAAGTGGTTTATGTAGTCTTTGCCTGCTTCATAA | 762 |
| 66944119 | 66833925 | TTCCCTAGGCATTCTATGCTATTCTGTACT | |
| chrX: 66763874- | chrX: 66833506- | ACATGGATCCATCAGTAAAATGTGGAGGGCTTAAGGATAAAAAGGCTTAAGTACAATCTCTGGCCCTACATTTTCTAAATGTTATGCCAC | 763 |
| 66944119 | 66833625 | CCTGACCAAGGGGCAACTCCTACAAAGCCA | |
| chrX: 66763874- | chrX: 66833986- | GCTGGGAAATAAATGAAGTTAAATAAATTTAGGCCATCGTGGTTTAAAGAATGGATTGTGGAGATAAGAAGGATAAAGGAAACCCAGAGT | 764 |
| 66944119 | 66834105 | CAAGAAAAATAAAACTTTTCATTGgtgcca | |
| chrX: 66763874- | chrX: 66836433- | TTCCCAGGAGAAAGTGGCTGAAGATTCCAGAGAGAAGCTGAATGCAGTTTAATTCTTTTTGCCATAAACACGACAACCCATTTTCCTGCA | 765 |
| 66944119 | 66836552 | AGCTGTGTTAGTTTGCTCTCTTCTTGGTTC | |
| chrX: 66763874- | chrX: 66836773- | CATCCTCCTAAGGCCTCAGAAATAAGGCCTTATTTTAATAAGTGCAAGTCAGTCATTTGAAGACTAAATCATAGAATCCTAGAAAACTAG | 766 |
| 66944119 | 66836892 | TACCGGGAGCAAGGCAAAAGAATGGGATGA | |
| chrX: 66763874- | chrX: 66836833- | AGACTAAATCATAGAATCCTAGAAAACTAGTACCGGGAGCAAGGCAAAAGAATGGGATGAGCATGAAACATATATTCAGAAGTTGTGGTG | 767 |
| 66944119 | 66836952 | TGTAGGTATATAAGCCAAGCTCTTTTCTTC | |
| chrX: 66763874- | chrX: 66836713- | TTGAAGGCAAAGCGCCTGGTGCTCCTGATCTCATGAGCACAGAGCATTTAGCCTAAGTCTCATCCTCCTAAGGCCTCAGAAATAAGGCCT | 768 |
| 66944119 | 66836832 | TATTTTAATAAGTGCAAGTCAGTCATTTGA | |
| chrX: 66763874- | chrX: 66837306- | TTTATGAAGTGTTGTGCATATTGTTGTTAATTTTAAGCTGTTACGTTAAAGAACCCCTAATCCAACTCTCTTGAGTTTTATAGATATCAT | 769 |
| 66944119 | 66837425 | AGAAGATATATCTTCCCTTGACATAGAAGC | |
| chrX: 66763874- | chrX: 66837814- | GCTTGTTTACCACATTGAATACTTTATCTGTGTTTATCTAACGACAGTTCCACCAGCTCTTTACCACTTGACTTTTGCCTAATTCAAAAA | 770 |
| 66944119 | 66837933 | TATACCAACTATGAAACATTTTCCTTCTCA | |
| chrX: 66763874- | chrX: 66837874- | TTACCACTTGACTTTTGCCTAATTCAAAAATATACCAACTATGAAACATTTTCCTTCTCAGTTTTTATTCTAGATTACATTTTGTTCAAC | 771 |
| 66944119 | 66837993 | TTTATCTTAATGTGTagtgtagaaagagta | |
| chrX: 66763874- | chrX: 66837634- | tcccaactcccactccAGTATACTAAGGCAGATTCCAGAGAAGAAACAGTGGAGAGCAGGCACTGATGAGGGACAAAGAAAAGCAGGCTC | 772 |
| 66944119 | 66837753 | CGTCTGGCTGCAACTTGTCTCTTCATGGCA | |
| chrX: 66763874- | chrX: 66837754- | AAAAGAAACTAGGAAAGTGCTATGCCAGAGACGACATGATAACTTTGCAGAATGGAAAGAGCTTGTTTACCACATTGAATACTTTATCTG | 773 |
| 66944119 | 66837873 | TGTTTATCTAACGACAGTTCCACCAGCTCT | |
| chrX: 66763874- | chrX: 66837694- | CACTGATGAGGGACAAAGAAAAGCAGGCTCCGTCTGGCTGCAACTTGTCTCTTCATGGCAAAAAGAAACTAGGAAAGTGCTATGCCAGAG | 774 |
| 66944119 | 66837813 | ACGACATGATAACTTTGCAGAATGGAAAGA | |
| chrX: 66763874- | chrX: 66838512- | GTTCCTATTCTATCTAGGGATAGAGTCAGGAAGGGCTTCATTGAATAAGTGGTAGCCTCTTGGGCTGAGACCTGAGTTATGAGATGATGT | 775 |
| 66944119 | 66838631 | GGCAAAGGAGACAGATGGCTGGGGGCAAGG | |
| chrX: 66763874- | chrX: 66838212- | TTGTTCAGGGTCCATCTTGGCCTTCAAATTAAGATGCCCTTTGAGAGATAACATTGTTGTTTTCAAACTCTGTTCTGTGACTTAAGAATG | 776 |
| 66944119 | 66838331 | AGAGGAGAAGGAAGAAAAGAGGAGAAAATT | |
| chrX: 66763874- | chrX: 66838452- | TACCGTATAATGCACCAAGCTAATAGGTGCTTTGAAAGAAGACCATACAAGTGGAGATGTGTTCCTATTCTATCTAGGGATAGAGTCAGG | 777 |
| 66944119 | 66838571 | AAGGGCTTCATTGAATAAGTGGTAGCCTCT | |
| chrX: 66763874- | chrX: 66838392- | ACATGGTGGATGGGAATCAGATATGTGCATCAATTATTTGTGTTCCAATCCATATAGAAGTACCGTATAATGCACCAAGCTAATAGGTGC | 778 |
| 66944119 | 66838511 | TTTGAAAGAAGACCATACAAGTGGAGATGT | |
| chrX: 66763874- | chrX: 66838332- | TGAGGGAAAAGTGCCCAAGCAGCGTCAAGGCTAGACACTGGAAATTTATCAATGAAAGCCACATGGTGGATGGGAATCAGATATGTGCAT | 779 |
| 66944119 | 66838451 | CAATTATTTGTGTTCCAATCCATATAGAAG | |
| chrX: 66763874- | chrX: 66838272- | TTTCAAACTCTGTTCTGTGACTTAAGAATGAGAGGAGAAGGAAGAAAAGAGGAGAAAATTTGAGGGAAAAGTGCCCAAGCAGCGTCAAGG | 780 |
| 66944119 | 66838391 | CTAGACACTGGAAATTTATCAATGAAAGCC | |
| chrX: 66763874- | chrX: 66838572- | TGGGCTGAGACCTGAGTTATGAGATGATGTGGCAAAGGAGACAGATGGCTGGGGGCAAGGTGGGGTCATTGAAATTGGAGGCAGTAGCAA | 781 |
| 66944119 | 66838691 | TATAAGCAAAGCTACAGGGGCATGAAAAAG | |
| chrX: 66763874- | chrX: 66838692- | CAAGGTTAGATTAGTGAATTGCAACAGGGTGGTACTGCTGGAAGGTCACATGGAAAAGATTGTGAAGGTATTGAGATAAGAAGCTAGAAA | 782 |
| 66944119 | 66838811 | TAAGCTTTGAATGCCATCCTAGTACTTTGA | |
| chrX: 66763874- | chrX: 66838632- | TGGGGTCATTGAAATTGGAGGCAGTAGCAATATAAGCAAAGCTACAGGGGCATGAAAAAGCAAGGTTAGATTAGTGAATTGCAACAGGGT | 783 |
| 66944119 | 66838751 | GGTACTGCTGGAAGGTCACATGGAAAAGAT | |
| chrX: 66763874- | chrX: 66839234- | TAGTCACTGATTGACTGAGTGGATGGCAGTGATAGGTGGGGTGCGTTGAGGGAAGTGTATTACATTAAGTCCAGGATGACTCATGGTTTT | 784 |
| 66944119 | 66839353 | CTAAGTTGAGTCATTGGGGATTGCCATCCA | |
| chrX: 66763874- | chrX: 66839898- | GTCTAACCTTGGCTTCTTGGTTGTGGATTCTCGTCAACATTTCACTGCTACCCAAGTTGTGTCTGCTTACATGATGCTATCTTCCTTCTT | 785 |
| 66944119 | 66840017 | TTGGGTTTCTGAAGCCCTCAGACACTTGGC | |
| chrX: 66763874- | chrX: 66839838- | TGAGTATCTCAATGGGGCTGTTTTTCTAGTTCTTGTAGTTTCTTTGGGCCAACATGAAATGTCTAACCTTGGCTTCTTGGTTGTGGATTC | 786 |
| 66944119 | 66839957 | TCGTCAACATTTCACTGCTACCCAAGTTGT | |
| chrX: 66763874- | chrX: 66840198- | GGTAGCATGATGATTGCAGAGCTCACTGGACTGAAAGTCAGATGCTTTACCCGCCTAGACTCTAGTACCAAGGGGAAGATGGAGTGAGAT | 787 |
| 66944119 | 66840317 | GGGGTAAATGGGGAGAaattaccatttatt | |
| chrX: 66763874- | chrX: 66839958- | GTCTGCTTACATGATGCTATCTTCCTTCTTTTGGGTTTCTGAAGCCCTCAGACACTTGGCTGAACATTTTTCACATTTCTTAAGCTATAT | 788 |
| 66944119 | 66840077 | CATCTGTGTTTTCCCTGCCACAGACAAAGT | |
| chrX: 66763874- | chrX: 66839778- | GCCTTGGGCCAGACCTCACTACACATCTGTTTAAGAGATCAGGGTAAGCTCTGTTCTTGGTGAGTATCTCAATGGGGCTGTTTTTCTAGT | 789 |
| 66944119 | 66839897 | TCTTGTAGTTTCTTTGGGCCAACATGAAAT | |
| chrX: 66763874- | chrX: 66839658- | TTTTTTAGTCTCTGCCAATGACTCCTCTCCCCATGATCAAAATCAGAAAATCAGTCTCTTATGTGTTGAGGAGTGAGACACTTCTCCCAA | 790 |
| 66944119 | 66839777 | GTGTTTAAGGCTAATACCTTGCCTTGTTTT | |
| chrX: 66763874- | chrX: 66840138- | GGGTAAGGGCAAGTGGTAAATGCTGCTTTTCTGCCTTAACCAGTAGTGTCTGACAGAGGAGGTAGCATGATGATTGCAGAGCTCACTGGA | 791 |
| 66944119 | 66840257 | CTGAAAGTCAGATGCTTTACCCGCCTAGAC | |
| chrX: 66763874- | chrX: 66840018- | TGAACATTTTTCACATTTCTTAAGCTATATCATCTGTGTTTTCCCTGCCACAGACAAAGTCACAAAAGGACTTTAAGATAGGTTTTGGTT | 792 |
| 66944119 | 66840137 | TTTTTTTTCCCCAGGGTTTTTATACATTTT | |
| chrX: 66763874- | chrX: 66839598- | tcagctgttattattGTGCTATATGTTGTGATGTGTACTGGAGTGAGATGGGGTAGGGGATTTTTTAGTCTCTGCCAATGACTCCTCTCC | 793 |
| 66944119 | 66839717 | CCATGATCAAAATCAGAAAATCAGTCTCTT | |
| chrX: 66763874- | chrX: 66840078- | CACAAAAGGACTTTAAGATAGGTTTTGGTTTTTTTTTTCCCCAGGGTTTTTATACATTTTGGGTAAGGGCAAGTGGTAAATGCTGCTTTT | 794 |
| 66944119 | 66840197 | CTGCCTTAACCAGTAGTGTCTGACAGAGGA | |
| chrX: 66763874- | chrX: 66839718- | ATGTGTTGAGGAGTGAGACACTTCTCCCAAGTGTTTAAGGCTAATACCTTGCCTTGTTTTGCCTTGGGCCAGACCTCACTACACATCTGT | 795 |
| 66944119 | 66839837 | TTAAGAGATCAGGGTAAGCTCTGTTCTTGG | |
| chrX: 66763874- | chrX: 66840802- | ACTGTAGTGTTTAATGCTTTTACCATCTGTTAAAATGATTTTGGAGTATAGCTAGATAACTGATGATGGTTGTTATATAGATTTTTTCAT | 796 |
| 66944119 | 66840921 | AGGTTGCCTGTTCCAAATTCTATGCCGTGG | |
| chrX: 66763874- | chrX: 66840862- | TGATGATGGTTGTTATATAGATTTTTTCATAGGTTGCCTGTTCCAAATTCTATGCCGTGGAAGAAGTTAAATATCCAGAATTTGACAGGA | 797 |
| 66944119 | 66840981 | AATATTATTCTACAACAGATCCCTGGCGta | |
| chrX: 66763874- | chrX: 66840742- | tttACCACATTTTCCAGAAAGAGGGTAGCTCCATAATGGGTGAGATACATTTTGGTGGCTACTGTAGTGTTTAATGCTTTTACCATCTGT | 798 |
| 66944119 | 66840861 | TAAAATGATTTTGGAGTATAGCTAGATAAC | |
| chrX: 66763874- | chrX: 66842142- | TGATGGCTAGAAAGCCATTGGAAAAAAAAAATTGGCTCACAGAAGACAGCAGATGTGGCTTGGGAAATGCAAGGACATGACTGTAATAAG | 799 |
| 66944119 | 66842261 | GATTTGTCTATCCAGCCCCATTTATGAGAG | |
| chrX: 66763874- | chrX: 66842262- | TGATTCCAGGAGAAAAGGACAGATTTGTATTGTCAGTGGGATACGCTGTTAAAAAACACTTTTGCTACTACCACTCCAGCTGTCTTGGCA | 800 |
| 66944119 | 66842381 | TGTTTGTTGGTGATGTAAGCTACAGAAAAT | |
| chrX: 66763874- | chrX: 66842022- | CATTTTCCTGTTTCTTTAGCAATGGATTCCAGAAACATAATGTGGAAATAGCTCTCAGTCCTTAGATTTGATGACATTGCAGAAAGAAAT | 801 |
| 66944119 | 66842141 | CTGGCTAGTCGTCCCATGGCTGATTGGCTA | |
| chrX: 66763874- | chrX: 66842382- | GGAAATCACCAATAGGGCTATAGCAACCTGATGCATAGTGACAAGTAATTGTTCTATTCATGGTTATGTGTTGTACAGAGCACTTGCTGC | 802 |
| 66944119 | 66842501 | ATGTCAGGTTTGAGACTTGAGTATGCATTA | |
| chrX: 66763874- | chrX: 66842202- | TGGGAAATGCAAGGACATGACTGTAATAAGGATTTGTCTATCCAGCCCCATTTATGAGAGTGATTCCAGGAGAAAAGGACAGATTTGTAT | 803 |
| 66944119 | 66842321 | TGTCAGTGGGATACGCTGTTAAAAAACACT | |
| chrX: 66763874- | chrX: 66841962- | GTGAGAAAAATATTGGTTCAGTAAGATTTAACAGGTAAATTAAAATCAAGTATTTGAAAACATTTTCCTGTTTCTTTAGCAATGGATTCC | 804 |
| 66944119 | 66842081 | AGAAACATAATGTGGAAATAGCTCTCAGTC | |
| chrX: 66763874- | chrX: 66842562- | TGTAAAATATTTAGTTTGGTCAGTCATAGGGCTGAGAACATGGTGGCAGTTACCTCCTAGTATCTGCAAGCAAAAAAAGTTTTTTCTTCC | 805 |
| 66944119 | 66842681 | TATAGCAATTGCCATCTCAGCCACTTTTGC | |
| chrX: 66763874- | chrX: 66842502- | GGGCCATGGACACCCCCATCTTATCTTTAAGTAGATTTCAAAGTAAATATTTGATGAATATGTAAAATATTTAGTTTGGTCAGTCATAGG | 806 |
| 66944119 | 66842621 | GCTGAGAACATGGTGGCAGTTACCTCCTAG | |
| chrX: 66763874- | chrX: 66842322- | TTTGCTACTACCACTCCAGCTGTCTTGGCATGTTTGTTGGTGATGTAAGCTACAGAAAATGGAAATCACCAATAGGGCTATAGCAACCTG | 807 |
| 66944119 | 66842441 | ATGCATAGTGACAAGTAATTGTTCTATTCA | |
| chrX: 66763874- | chrX: 66842082- | CTTAGATTTGATGACATTGCAGAAAGAAATCTGGCTAGTCGTCCCATGGCTGATTGGCTATGATGGCTAGAAAGCCATTGGAAAAAAAAA | 808 |
| 66944119 | 66842201 | ATTGGCTCACAGAAGACAGCAGATGTGGCT | |
| chrX: 66763874- | chrX: 66842442- | TGGTTATGTGTTGTACAGAGCACTTGCTGCATGTCAGGTTTGAGACTTGAGTATGCATTAGGGCCATGGACACCCCCATCTTATCTTTAA | 809 |
| 66944119 | 66842561 | GTAGATTTCAAAGTAAATATTTGATGAATA | |
| chrX: 66763874- | chrX: 66842873- | AAGTAGGAGTGACCTGTCTTCTCTCTTTCTTCACGATGGGGACTAGTGTGTGTATATAAGGGGATAATTTTTGTGTCACATAAAATATAA | 810 |
| 66944119 | 66842992 | CCTTACTTAGAAGGCAAGACTTCCAGAATG | |
| chrX: 66763874- | chrX: 66854496- | ACACTAAATTTCAAATCTCACGGTGGCAGGGAATAAAGATGCTACCTATCTTAAGCCATTACTTCACCAACTTCTCCACCAAAATATTCC | 811 |
| 66944119 | 66854615 | TTGTAACCACAAATAAGTAAGCACAATAGA | |
| chrX: 66763874- | chrX: 66854616- | TCTATAAGGAGAGAATAATTGTGAACTCTGATTTTATCTTAAAAAGTCATGTAGGGATGTCATGTTCCACAATGTGATTAATAAAATATA | 812 |
| 66944119 | 66854735 | TTTTGTTACTAAACACAAGGAAAAATATTA | |
| chrX: 66763874- | chrX: 66854556- | ACTTCACCAACTTCTCCACCAAAATATTCCTTGTAACCACAAATAAGTAAGCACAATAGATCTATAAGGAGAGAATAATTGTGAACTCTG | 813 |
| 66944119 | 66854675 | ATTTTATCTTAAAAAGTCATGTAGGGATGT | |
| chrX: 66763874- | chrX: 66854676- | CATGTTCCACAATGTGATTAATAAAATATATTTTGTTACTAAACACAAGGAAAAATATTATGTTCCATAAAGATGTTTGGTGGTTGCCTC | 814 |
| 66944119 | 66854795 | GACCTCTTTTAGTTTGAAAAGTAGGTATGT | |
| chrX: 66763874- | chrX: 66854436- | ATGAACTAAATTTCGGTAATACTTTTAATAGTAAACATTGCTGCCCTCGTGAATGAACACACACTAAATTTCAAATCTCACGGTGGCAGG | 815 |
| 66944119 | 66854555 | GAATAAAGATGCTACCTATCTTAAGCCATT | |
| chrX: 66763874- | chrX: 66854888- | CCCCGCAACCCCCACATGTATTTACCTTTCTCTAAAAGCTCTGCATAGCCAAGAAAAGTGCTCTTTTTTATTTTTAGGATATTAGATATT | 816 |
| 66944119 | 66855007 | TCATTTTCTTATGGTAAGACAAAAGATTAA | |
| chrX: 66763874- | chrX: 66855265- | TGGTCTGTGACTTTTGCATTTCACCCCGCTTCCACAGTCACTGGTGGGACTTACTTAAGTTAATCAGATTCTTCAAAGTATCCCCAAGTC | 817 |
| 66944119 | 66855384 | CTCCTTTGAAAAGAAAGTTGGGGGACAGGA | |
| chrX: 66763874- | chrX: 66855451- | GGTGATCTCAGCTGGGTGCCAAGGTTTCCTAAGCCCAAGTTCCCCATGGTTGAGCCTGTATTGTCAGGCCAACAGCTTCTAGTAATCCAC | 818 |
| 66944119 | 66855570 | TTTTATTTAATTAATAGTGAAACTGTTGAA | |
| chrX: 66763874- | chrX: 66855751- | CTGTGTTTGCAATTAGGATGTTATCTAGGAGCATATTCAAAACTTTTGAGGTTTTTATTTTAGTTTTTCTTTCATTATGTGCTGTTTTAG | 819 |
| 66944119 | 66855870 | TAATATCAAAGAATACATGTAATATATAAT | |
| chrX: 66763874- | chrX: 66855691- | GGTAAGAAAAAACACAATGTCACGTGCAAATTCTGCACTTGTTCTCAAAGCAAACCTCTCCTGTGTTTGCAATTAGGATGTTATCTAGGA | 820 |
| 66944119 | 66855810 | GCATATTCAAAACTTTTGAGGTTTTTATTT | |
| chrX: 66763874- | chrX: 66855571- | GAATTGCAAGTGGTGTTCTGGTTCAGAAACCTTCCGTTCTATGGGGCACTGCTTTTGCTTCAGATTCATAAAACCAAATGCTCTGCCTCA | 821 |
| 66944119 | 66855690 | AGATAATAAGTGAACGTGTAACCCTCGGGA | |
| chrX: 66763874- | chrX: 66855811- | TAGTTTTTCTTTCATTATGTGCTGTTTTAGTAATATCAAAGAATACATGTAATATATAATTTATATGTCATAACAATAAAATTAATGTTG | 822 |
| 66944119 | 66855930 | ATGAGCCCAGATTAAAGAATCAACAACATT | |
| chrX: 66763874- | chrX: 66855511- | TTGTCAGGCCAACAGCTTCTAGTAATCCACTTTTATTTAATTAATAGTGAAACTGTTGAAGAATTGCAAGTGGTGTTCTGGTTCAGAAAC | 823 |
| 66944119 | 66855630 | CTTCCGTTCTATGGGGCACTGCTTTTGCTT | |
| chrX: 66763874- | chrX: 66855631- | CAGATTCATAAAACCAAATGCTCTGCCTCAAGATAATAAGTGAACGTGTAACCCTCGGGAGGTAAGAAAAAACACAATGTCACGTGCAAA | 824 |
| 66944119 | 66855750 | TTCTGCACTTGTTCTCAAAGCAAACCTCTC | |
| chrX: 66763874- | chrX: 66856551- | GCTCATGAGTCAGAGCTGCAATGTGCCCTTTGTCCACACTAGGTCAGGATCAGTGGGAGTGCTACCCAAAATATTTTGCTAGCTGGGGAG | 825 |
| 66944119 | 66856670 | TCAGGGAGAAGCAGAGACTGACCTAGTGAG | |
| chrX: 66763874- | chrX: 66856671- | GCCAGGAGGCACTATCTCAGGTCTCTAGTCAAAATGGGTTGCAATTAGTAAAAGTCCAGATTCTGAATCCCCTTCACTATTTATCTTCCT | 826 |
| 66944119 | 66856790 | CTTCCTCCTTTACAGTTATTTTTGTTCAAG | |
| chrX: 66763874- | chrX: 66856731- | TTCTGAATCCCCTTCACTATTTATCTTCCTCTTCCTCCTTTACAGTTATTTTTGTTCAAGGTGCACTTTATTAAACTCATGCCTAACAAA | 827 |
| 66944119 | 66856850 | CAAAACTCTAATGAATATTTTGTCTTTCAT | |
| chrX: 66763874- | chrX: 66856611- | GCTACCCAAAATATTTTGCTAGCTGGGGAGTCAGGGAGAAGCAGAGACTGACCTAGTGAGGCCAGGAGGCACTATCTCAGGTCTCTAGTC | 828 |
| 66944119 | 66856730 | AAAATGGGTTGCAATTAGTAAAAGTCCAGA | |
| chrX: 66763874- | chrX: 66856791- | GTGCACTTTATTAAACTCATGCCTAACAAACAAAACTCTAATGAATATTTTGTCTTTCATTGATTGTAAATTCAATTAATTAGATTGCTT | 829 |
| 66944119 | 66856910 | GAAAAAATTTTAACTGTATTTTCACTTTAG | |
| chrX: 66763874- | chrX: 66861277- | TTTCTATTTCTTCTCCATGTTTCATTCAGGACTGAGGAAGGGGGCACAGTTTTTACCCAAGGAAATGACATTTTTAGCCAAAAGAAATGA | 830 |
| 66944119 | 66861396 | TCTTAGCATTTAGCTGAATTATATATTGGA | |
| chrX: 66763874- | chrX: 66861457- | TTGCTGGCTTTCTAGTTAGGGTAGGGAAAGGAAGGCTTGTGGGGAATGAAGATAGGCCATGATATCAAGCCACTGGGTTTGCAAATCAGT | 831 |
| 66944119 | 66861576 | AGAATTTTTTATTGCTTTCTGTTGTACTTG | |
| chrX: 66763874- | chrX: 66861637- | AGTTTTCTTTGCTCTTGTCCTGACATAGCTGTTCACTTGGGGTTGAGGGGAGGATAACCTTTCATGTTTTTTTTTTTTCTTCATTCTGAT | 832 |
| 66944119 | 66861756 | GACTGTGCTGAACATTCAAACCAAAAGGCC | |
| chrX: 66763874- | chrX: 66861337- | GGAAATGACATTTTTAGCCAAAAGAAATGATCTTAGCATTTAGCTGAATTATATATTGGAAGTAAGCTCCTTCCATGTGGAACTTATGGC | 833 |
| 66944119 | 66861456 | CTTGCTAGCCTTGGTTTGTTGGAAGTGCTC | |
| chrX: 66763874- | chrX: 66861397- | AGTAAGCTCCTTCCATGTGGAACTTATGGCCTTGCTAGCCTTGGTTTGTTGGAAGTGCTCTTGCTGGCTTTCTAGTTAGGGTAGGGAAAG | 834 |
| 66944119 | 66861516 | GAAGGCTTGTGGGGAATGAAGATAGGCCAT | |
| chrX: 66763874- | chrX: 66861517- | GATATCAAGCCACTGGGTTTGCAAATCAGTAGAATTTTTTATTGCTTTCTGTTGTACTTGGGACTTGAATAAAGGCTGATATTTGTGTCT | 835 |
| 66944119 | 66861636 | TGCTGGTAAAGTGCTTGTAAAGTGAGTGAA | |
| chrX: 66763874- | chrX: 66861217- | AGTTTGTGTTAGTAACAGAAGTCAAAAAGGTGGAGAAAGGAGAAAGGTACTTGTGAAAATTTTCTATTTCTTCTCCATGTTTCATTCAGG | 836 |
| 66944119 | 66861336 | ACTGAGGAAGGGGGCACAGTTTTTACCCAA | |
| chrX: 66763874- | chrX: 66861577- | GGACTTGAATAAAGGCTGATATTTGTGTCTTGCTGGTAAAGTGCTTGTAAAGTGAGTGAAAGTTTTCTTTGCTCTTGTCCTGACATAGCT | 837 |
| 66944119 | 66861696 | GTTCACTTGGGGTTGAGGGGAGGATAACCT | |
| chrX: 66763874- | chrX: 66861697- | TTCATGTTTTTTTTTTTTCTTCATTCTGATGACTGTGCTGAACATTCAAACCAAAAGGCCATTGGTGGAAAGTAAAGGTGAGTGGTGAGA | 838 |
| 66944119 | 66861816 | AGACAATAGGGTAATGGAAACTGTGTTGGA | |
| chrX: 66763874- | chrX: 66862358- | CCAGAATCATTGTACATCATTTTTTCAACAGAAGCTTCAGGCATAGGGATTATGCTTGGTACTTTATGTTGTGGAATGGAATCTGGCGGA | 839 |
| 66944119 | 66862477 | TGTCCATGTGATCTATAGAAACACCTAAGG | |
| chrX: 66763874- | chrX: 66862238- | CTTCTCCTTCATATCCACCTGAAAGAGCTTGGGCGCAGAAGTTCTTGCAGAAAGGCAGTTAGACAAGGTGACTTCTGAAGCTCCAGTGGC | 840 |
| 66944119 | 66862357 | CAAGTATTTTGATGGTAGCCTAAAAGATGT | |
| chrX: 66763874- | chrX: 66862298- | AGACAAGGTGACTTCTGAAGCTCCAGTGGCCAAGTATTTTGATGGTAGCCTAAAAGATGTCCAGAATCATTGTACATCATTTTTTCAACA | 841 |
| 66944119 | 66862417 | GAAGCTTCAGGCATAGGGATTATGCTTGGT | |
| chrX: 66763874- | chrX: 66861938- | ACAAACAGGCTCCCATTAGGGCTCATTTTCCTTCATTCCTTAGTAAGGAAGAAGTGCTTATAAAATATAGCAGTTGTGCTCTTGTGAATG | 842 |
| 66944119 | 66862057 | ATAGCATGGGCAGTTGTCATCTCCCTGAAG | |
| chrX: 66763874- | chrX: 66862178- | TTTACTATAGGAGACTGAGAATTAACCTTCCATGAAGGTTTTAGGATTGGCTTTCTGGCCCTTCTCCTTCATATCCACCTGAAAGAGCTT | 843 |
| 66944119 | 66862297 | GGGCGCAGAAGTTCTTGCAGAAAGGCAGTT | |
| chrX: 66763874- | chrX: 66862118- | TGACAAAAGCTGACCTTTGGGTAGTGAGAACAATGTTCCATTTTGTTCAAACTTGAATTTTTTACTATAGGAGACTGAGAATTAACCTTC | 844 |
| 66944119 | 66862237 | CATGAAGGTTTTAGGATTGGCTTTCTGGCC | |
| chrX: 66763874- | chrX: 66862058- | CAGATGTAACCCAGAATGTCACTTGAGTTTTGTTTAATGCTTAGGCATAAGACATAGGAATGACAAAAGCTGACCTTTGGGTAGTGAGAA | 845 |
| 66944119 | 66862177 | CAATGTTCCATTTTGTTCAAACTTGAATTT | |
| chrX: 66763874- | chrX: 66862418- | ACTTTATGTTGTGGAATGGAATCTGGCGGATGTCCATGTGATCTATAGAAACACCTAAGGAAAGTGAAGAAATGAGGGAAAAAAAAGAAC | 846 |
| 66944119 | 66862537 | AAGACTTTTATGATAATACTAATCACGATC | |
| chrX: 66763874- | chrX: 66861998- | TAAAATATAGCAGTTGTGCTCTTGTGAATGATAGCATGGGCAGTTGTCATCTCCCTGAAGCAGATGTAACCCAGAATGTCACTTGAGTTT | 847 |
| 66944119 | 66862117 | TGTTTAATGCTTAGGCATAAGACATAGGAA | |
| chrX: 66763874- | chrX: 66864300- | AACCTTTCCTTCTATAAGAGCTATAAACCTTGAGAACAGTCTTAAAACATAGGTATGTAGGCCACACCATTCACCACGAATGTACTGATA | 848 |
| 66944119 | 66864419 | CTCATCAGAATATGGAAGAAGCACCAGAGA | |
| chrX: 66763874- | chrX: 66862980- | TTGAGCAATGAATAATAGTCATTTATGCCTGCAGGTTAATGCTGAAGACCTGAGACTTCACTTGCCTATTTCTGCCATTCAGTGACATGT | 849 |
| 66944119 | 66863099 | GTTGCATTGGTTTTTTGTGTCTTTCCAGTT | |
| chrX: 66763874- | chrX: 66863940- | GTTTTATTTAGCTTAGAACAACTCATGTCTGCTCAACCTCTAGAGGCGCTCAGCCCACATTCTGCAGTAGAAACTCCCATTTTCAGGCCT | 850 |
| 66944119 | 66864059 | CTTATATACGGTAATGTCTCCTTCCTCTAA | |
| chrX: 66763874- | chrX: 66863340- | TCTCAGAAATAGAGTCATTGGCAAGGCCCTATCAAATAACTTAGGAGCCTAAGGAAGCAAATTTTTGTACTTGCTAGTTCCCTGGTTTCA | 851 |
| 66944119 | 66863459 | GCAGCCTTGTTTGTACAGGCAATTTAGGCA | |
| chrX: 66763874- | chrX: 66863400- | ATTTTTGTACTTGCTAGTTCCCTGGTTTCAGCAGCCTTGTTTGTACAGGCAATTTAGGCAGTGAAGGTGGTCCCAGCTGGGGCTTGGGGC | 852 |
| 66944119 | 66863519 | TCAGTGGGTCCTAGAAATGAAAGAAAAATT | |
| chrX: 66763874- | chrX: 66862920- | CACCCTACAACCATCATCTTTAGGAGAAGTCTCAGAAAATTCAGCTTCACACTAACTAACTTGAGCAATGAATAATAGTCATTTATGCCT | 853 |
| 66944119 | 66863039 | GCAGGTTAATGCTGAAGACCTGAGACTTCA | |
| chrX: 66763874- | chrX: 66865260- | GGATCCTGTGCCATTTGAGAACAGGGAAGAAAAGAAATGAGGTTTTGGGGAGGGAATCACCCAACTCACAGAACACACAGAAATCCAGCA | 854 |
| 66944119 | 66865379 | AGGTTTCAAAACGCTCTACACCTTAGAGTC | |
| chrX: 66763874- | chrX: 66862800- | AGATATCTTAACCTCAGGCTTCCTGCCTTCATTGCTCCCGCATATAGACATAGACTATGAGATTGGCTAATCCCAGAGAACTTCCCTAAT | 855 |
| 66944119 | 66862919 | CCCTTGGCAAGATCCAAAAAGGCTCAGTCA | |
| chrX: 66763874- | chrX: 66864420- | GTTTGAAGCATCTAGAGAAAAGGTAGAAAGAGAATGCCCTTTAACTGACCTCCTCAGTGATAGCCAATCACAATGATGAGTGTTGATTCA | 856 |
| 66944119 | 66864539 | TCATTTTGGCTAGGTGGCAGAAATATCTAT | |
| chrX: 66763874- | chrX: 66863460- | GTGAAGGTGGTCCCAGCTGGGGCTTGGGGCTCAGTGGGTCCTAGAAATGAAAGAAAAATTAATGATTTGAAAAGATTTAATTTCCTCCCT | 857 |
| 66944119 | 66863579 | TCTTGTTTTCTACTCTGCTGGCTAGTAAAG | |
| chrX: 66763874- | chrX: 66865200- | GAGGGAGGCAGGCAGAAGGGCAACTGGCAGGGCTGCCTGGGAGGAGCTCTGCAATGAGGTGGATCCTGTGCCATTTGAGAACAGGGAAGA | 858 |
| 66944119 | 66865319 | AAAGAAATGAGGTTTTGGGGAGGGAATCAC | |
| chrX: 66763874- | chrX: 66865320- | CCAACTCACAGAACACACAGAAATCCAGCAAGGTTTCAAAACGCTCTACACCTTAGAGTCTGTTAAGTTAGGGAAACTCTGTGAGCTCAT | 859 |
| 66944119 | 66865439 | AGGGCCAAATGCACTTGCCTGCTTGAAATA | |
| chrX: 66763874- | chrX: 66863760- | TGGCCTCCTTGGGACCTGTCTTTGCATTAATAGTTCCTAGGTAGGTAAGAACTCAGAGTGAAGAAACACATTTATTCTCCTCTCCAGAGA | 860 |
| 66944119 | 66863879 | CCTGATCTCAAAGCCTGTCCATTAGTCCCT | |
| chrX: 66763874- | chrX: 66864780- | GAAAGGATGGCTGTCAGAAAAGGAAATGAGGATGGGTTCCAGAGACTTCAGACCACCCCAACTTCCCCAGTGAGACCCTGGCACCTCCCC | 861 |
| 66944119 | 66864899 | ATACCCTCTCACCTAGCGGGCCCTGTCTAT | |
| chrX: 66763874- | chrX: 66864960- | GCACCACAGTAATAGCAGCCATATCAGATGGGAAAGGAGTTCAAGTGAACAAACAAGCAAATTCAATAGTCAGATAGATTAGATTATACT | 862 |
| 66944119 | 66865079 | TGATGCTTCCTCTGAGTTTTACAAATATGG | |
| chrX: 66763874- | chrX: 66863820- | AAGAAACACATTTATTCTCCTCTCCAGAGACCTGATCTCAAAGCCTGTCCATTAGTCCCTAACCTTAATCTAAGGTAGCATCTTATATCT | 863 |
| 66944119 | 66863939 | GGCTAAATTGGCTCAAGCCCTAGCTCCTTA | |
| chrX: 66763874- | chrX: 66864240- | CCTTTCTCTTTCCTCTCACTGGCTGGAAGTGTTGAGTTCCACTTCAGAACCAGAACAGAGAACCTTTCCTTCTATAAGAGCTATAAACCT | 864 |
| 66944119 | 66864359 | TGAGAACAGTCTTAAAACATAGGTATGTAG | |
| chrX: 66763874- | chrX: 66864540- | AAAACAGAAGCTGCCATGTTGTTTTCTTCCAGTCCTCAGGGCCTACAAGAAGGCAGCTATCATTTGGTATTACTGAAAACATGCCCCATG | 865 |
| 66944119 | 66864659 | TTCAGCTCATACCCCCAAATTACCCATTGC | |
| chrX: 66763874- | chrX: 66863580- | GAAAAATTTGTCCTTATTAGAGAGGTTAGAAGTGGAGAAACCCCAACTGAGTCCCCAGCCTGTTCCTTGGGATGAATATGAGACTGTTCC | 866 |
| 66944119 | 66863699 | TTAGCAAAGGCTTCCTGGCCTCGGCCCCAG | |
| chrX: 66763874- | chrX: 66864480- | TAGCCAATCACAATGATGAGTGTTGATTCATCATTTTGGCTAGGTGGCAGAAATATCTATAAAACAGAAGCTGCCATGTTGTTTTCTTCC | 867 |
| 66944119 | 66864599 | AGTCCTCAGGGCCTACAAGAAGGCAGCTAT | |
| chrX: 66763874- | chrX: 66864900- | AGAGCAGAGAATGAAACAGAGCACTCATCTAGAGGTAGTGTGTCAGCAAGCCCAGGCACTGCACCACAGTAATAGCAGCCATATCAGATG | 868 |
| 66944119 | 66865019 | GGAAAGGAGTTCAAGTGAACAAACAAGCAA | |
| chrX: 66763874- | chrX: 66862860- | GATTGGCTAATCCCAGAGAACTTCCCTAATCCCTTGGCAAGATCCAAAAAGGCTCAGTCACACCCTACAACCATCATCTTTAGGAGAAGT | 869 |
| 66944119 | 66862979 | CTCAGAAAATTCAGCTTCACACTAACTAAC | |
| chrX: 66763874- | chrX: 66864360- | GCCACACCATTCACCACGAATGTACTGATACTCATCAGAATATGGAAGAAGCACCAGAGAGTTTGAAGCATCTAGAGAAAAGGTAGAAAG | 870 |
| 66944119 | 66864479 | AGAATGCCCTTTAACTGACCTCCTCAGTGA | |
| chrX: 66763874- | chrX: 66864600- | CATTTGGTATTACTGAAAACATGCCCCATGTTCAGCTCATACCCCCAAATTACCCATTGCTACTGTTTATGCTGGGCTAATATGAAGCCC | 871 |
| 66944119 | 66864719 | AGGGCCCTAATGTCTAGGTCTAGGCAGTAA | |
| chrX: 66763874- | chrX: 66864060- | CCACCCAGGGCTTAAGCTTCCTGCTTATCCACTTCACCCTGTATTGAGGGCTTTCTTCTCAAAGAGACATTGATGAGGAGCCCCTAGAGA | 872 |
| 66944119 | 66864179 | GAGATGCTGTGCTCTGGGACCAGACCCCTT | |
| chrX: 66763874- | chrX: 66864660- | TACTGTTTATGCTGGGCTAATATGAAGCCCAGGGCCCTAATGTCTAGGTCTAGGCAGTAAGGCCTAGAGCAGTGCCTAAAGAGCCTGAGA | 873 |
| 66944119 | 66864779 | GCAGTGCCTTCCTTTCTTCAGAGTACTCAT | |
| chrX: 66763874- | chrX: 66863220- | GCAAGGTCTTCTTCAAAAGAGCCGCTGAAGGTAAAGGGTCTTGCACATGCACTTCTCTTTCCCTTTCTCCTTTACCTTCCAGAGAGAGAC | 874 |
| 66944119 | 66863339 | ACTAACCTTTCAGGGCCCAGGATTTTATCA | |
| chrX: 66763874- | chrX: 66862680- | ATCACTTTGATATTTGGTCTGGTTTTGTTATAGGTCTCCCTTTGGATGAGGTAAAGTTACAAACCTGGGTTCATATCATTTAATTAGTCT | 875 |
| 66944119 | 66862799 | GAAAATGTTGCCTGGACACCACCTTCAGTT | |
| chrX: 66763874- | chrX: 66864000- | TCTGCAGTAGAAACTCCCATTTTCAGGCCTCTTATATACGGTAATGTCTCCTTCCTCTAACCACCCAGGGCTTAAGCTTCCTGCTTATCC | 876 |
| 66944119 | 66864119 | ACTTCACCCTGTATTGAGGGCTTTCTTCTC | |
| chrX: 66763874- | chrX: 66864720- | GGCCTAGAGCAGTGCCTAAAGAGCCTGAGAGCAGTGCCTTCCTTTCTTCAGAGTACTCATGAAAGGATGGCTGTCAGAAAAGGAAATGAG | 877 |
| 66944119 | 66864839 | GATGGGTTCCAGAGACTTCAGACCACCCCA | |
| chrX: 66763874- | chrX: 66865080- | GTCACTAAATTGTTATTTTCAGAAAACAGGGGAAATGCTCAATCACATTGTGAAAGGGAAGATTTTGCTGTCATATCATACATCCCACAT | 878 |
| 66944119 | 66865199 | GGGAGCTTTCTGCAGAAGTTAGAGCTGAAG | |
| chrX: 66763874- | chrX: 66865380- | TGTTAAGTTAGGGAAACTCTGTGAGCTCATAGGGCCAAATGCACTTGCCTGCTTGAAATATGAAAAATCAGCAATGGATTCCTTGAAAAA | 879 |
| 66944119 | 66865499 | CAATGAAAAGGGAACCTTCTGAGCCCCTTG | |
| chrX: 66763874- | chrX: 66865440- | TGAAAAATCAGCAATGGATTCCTTGAAAAACAATGAAAAGGGAACCTTCTGAGCCCCTTGGTTATTTTGACATATGGACCATAGATTTCA | 880 |
| 66944119 | 66865559 | GTCCTGAGCCCTTTGAAGGTAGGAGAAGGT | |
| chrX: 66763874- | chrX: 66863100- | TGGAGACTGCCAGGGACCATGTTTTGCCCATTGACTATTACTTTCCACCCCAGAAGACCTGCCTGATCTGTGGAGATGAAGCTTCTGGGT | 881 |
| 66944119 | 66863219 | GTCACTATGGAGCTCTCACATGTGGAAGCT | |
| chrX: 66763874- | chrX: 66865020- | ATTCAATAGTCAGATAGATTAGATTATACTTGATGCTTCCTCTGAGTTTTACAAATATGGGTCACTAAATTGTTATTTTCAGAAAACAGG | 882 |
| 66944119 | 66865139 | GGAAATGCTCAATCACATTGTGAAAGGGAA | |
| chrX: 66763874- | chrX: 66863160- | GCCTGATCTGTGGAGATGAAGCTTCTGGGTGTCACTATGGAGCTCTCACATGTGGAAGCTGCAAGGTCTTCTTCAAAAGAGCCGCTGAAG | 883 |
| 66944119 | 66863279 | GTAAAGGGTCTTGCACATGCACTTCTCTTT | |
| chrX: 66763874- | chrX: 66863640- | TGTTCCTTGGGATGAATATGAGACTGTTCCTTAGCAAAGGCTTCCTGGCCTCGGCCCCAGAAAGGGAGTGTTCTCACTCTTCAGCAGACT | 884 |
| 66944119 | 66863759 | ATCAGTCTCTGCACCTGCTCCCTCCTGTTG | |
| chrX: 66763874- | chrX: 66865140- | GATTTTGCTGTCATATCATACATCCCACATGGGAGCTTTCTGCAGAAGTTAGAGCTGAAGGAGGGAGGCAGGCAGAAGGGCAACTGGCAG | 885 |
| 66944119 | 66865259 | GGCTGCCTGGGAGGAGCTCTGCAATGAGGT | |
| chrX: 66763874- | chrX: 66864180- | GTTAAACACCAGTATTCACCTCTGCCCCAACTTTCCCCAAAGAGGTACTTCCTGCCAAGGCCTTTCTCTTTCCTCTCACTGGCTGGAAGT | 886 |
| 66944119 | 66864299 | GTTGAGTTCCACTTCAGAACCAGAACAGAG | |
| chrX: 66763874- | chrX: 66864840- | ACTTCCCCAGTGAGACCCTGGCACCTCCCCATACCCTCTCACCTAGCGGGCCCTGTCTATAGAGCAGAGAATGAAACAGAGCACTCATCT | 887 |
| 66944119 | 66864959 | AGAGGTAGTGTGTCAGCAAGCCCAGGCACT | |
| chrX: 66763874- | chrX: 66863880- | AACCTTAATCTAAGGTAGCATCTTATATCTGGCTAAATTGGCTCAAGCCCTAGCTCCTTAGTTTTATTTAGCTTAGAACAACTCATGTCT | 888 |
| 66944119 | 66863999 | GCTCAACCTCTAGAGGCGCTCAGCCCACAT | |
| chrX: 66763874- | chrX: 66863700- | AAAGGGAGTGTTCTCACTCTTCAGCAGACTATCAGTCTCTGCACCTGCTCCCTCCTGTTGTGGCCTCCTTGGGACCTGTCTTTGCATTAA | 889 |
| 66944119 | 66863819 | TAGTTCCTAGGTAGGTAAGAACTCAGAGTG | |
| chrX: 66763874- | chrX: 66863280- | CCCTTTCTCCTTTACCTTCCAGAGAGAGACACTAACCTTTCAGGGCCCAGGATTTTATCATCTCAGAAATAGAGTCATTGGCAAGGCCCT | 890 |
| 66944119 | 66863399 | ATCAAATAACTTAGGAGCCTAAGGAAGCAA | |
| chrX: 66763874- | chrX: 66863520- | AATGATTTGAAAAGATTTAATTTCCTCCCTTCTTGTTTTCTACTCTGCTGGCTAGTAAAGGAAAAATTTGTCCTTATTAGAGAGGTTAGA | 891 |
| 66944119 | 66863639 | AGTGGAGAAACCCCAACTGAGTCCCCAGCC | |
| chrX: 66763874- | chrX: 66864120- | AAAGAGACATTGATGAGGAGCCCCTAGAGAGAGATGCTGTGCTCTGGGACCAGACCCCTTGTTAAACACCAGTATTCACCTCTGCCCCAA | 892 |
| 66944119 | 66864239 | CTTTCCCCAAAGAGGTACTTCCTGCCAAGG | |
| chrX: 66763874- | chrX: 66863040- | CTTGCCTATTTCTGCCATTCAGTGACATGTGTTGCATTGGTTTTTTGTGTCTTTCCAGTTTGGAGACTGCCAGGGACCATGTTTTGCCCA | 893 |
| 66944119 | 66863159 | TTGACTATTACTTTCCACCCCAGAAGACCT | |
| chrX: 66763874- | chrX: 66862740- | AAACCTGGGTTCATATCATTTAATTAGTCTGAAAATGTTGCCTGGACACCACCTTCAGTTAGATATCTTAACCTCAGGCTTCCTGCCTTC | 894 |
| 66944119 | 66862859 | ATTGCTCCCGCATATAGACATAGACTATGA | |
| chrX: 66763874- | chrX: 66865813- | GCTTGTTGAAACATCAAATTATACCTGTCTTAGAGAAAATAGAAACAAAAATCTTTCTCTTCCTTACTTGCTTGTTGTAGTCAGTTAACT | 895 |
| 66944119 | 66865932 | CGGACTGAGTATTCAGAGTCTTGATTATCA | |
| chrX: 66763874- | chrX: 66865633- | TCTTTCTCCTCCCATCTGTGAAGCTGTTGGATTGATTTTACTGCCATCATTATCCCTGTTTGAAGGCAGGGGGCTGTCTTATTACCCAAA | 896 |
| 66944119 | 66865752 | GAGGACATTTATTGATTTGGTTTTCTTTTT | |
| chrX: 66763874- | chrX: 66866173- | GTGAGGGTGAGGCTGGGCAGGGAGGGTCTGCATAGAAAAAAGGGTGCGGTGAGAAAAAATAATGCTACTAAGCCATGAGGGTAAAATGAC | 897 |
| 66944119 | 66866292 | CAAATTCTGGTTGAGAGAAACTTGGTCAAA | |
| chrX: 66763874- | chrX: 66866113- | GGAAGAGTGGGAAGAGGGAGTCCTACATTTTCTCCTTGTCAGTAATGTTGGAGAATTGGGGTGAGGGTGAGGCTGGGCAGGGAGGGTCTG | 898 |
| 66944119 | 66866232 | CATAGAAAAAAGGGTGCGGTGAGAAAAAAT | |
| chrX: 66763874- | chrX: 66865873- | TCCTTACTTGCTTGTTGTAGTCAGTTAACTCGGACTGAGTATTCAGAGTCTTGATTATCACTTAATTCATAGTTTCATAAATCTCTGGAA | 899 |
| 66944119 | 66865992 | TGGGCATAGGTACAGGACTTAAAAGCCTGG | |
| chrX: 66763874- | chrX: 66866353- | CTGGGTTAGAAACAGGCATGGAGGGAAATAGTTGGTTTATGGAGTGGGTAGGATGAGTGGGGTGGTGAAAGGGAAGGCATTTTGGATGCT | 901 |
| 66944119 | 66866472 | AAGAGACCAGGAAGTCAAAGCAAGGCAATA | |
| chrX: 66763874- | chrX: 66866053- | TGTCATTGGTGCAGGGCTCAGCACAGAGTCAGTTGTAATCTGGACAGGTTTTGTTGTTGAGGAAGAGTGGGAAGAGGGAGTCCTACATTT | 902 |
| 66944119 | 66866172 | TCTCCTTGTCAGTAATGTTGGAGAATTGGG | |
| chrX: 66763874- | chrX: 66865753- | CCATTTTTACAATGCATCTTTATCGCCCATATGGCCTTTCTGGAGGTGGTTTTCAGTCTGGCTTGTTGAAACATCAAATTATACCTGTCT | 903 |
| 66944119 | 66865872 | TAGAGAAAATAGAAACAAAAATCTTTCTCT | |
| chrX: 66763874- | chrX: 66865993- | CATCTCAGACAGAAATATGTTTTTAGCTTTGGTGGTTTATAACAGATGGGACTTTTAGGCTGTCATTGGTGCAGGGCTCAGCACAGAGTC | 904 |
| 66944119 | 66866112 | AGTTGTAATCTGGACAGGTTTTGTTGTTGA | |
| chrX: 66763874- | chrX: 66866233- | AATGCTACTAAGCCATGAGGGTAAAATGACCAAATTCTGGTTGAGAGAAACTTGGTCAAAGTGTGTATGGGGAGAGAAAGTTGGTCAAAG | 905 |
| 66944119 | 66866352 | TCTGTGTCTGAGTGCTTGGTGGGATGAACT | |
| chrX: 66763874- | chrX: 66865933- | CTTAATTCATAGTTTCATAAATCTCTGGAATGGGCATAGGTACAGGACTTAAAAGCCTGGCATCTCAGACAGAAATATGTTTTTAGCTTT | 906 |
| 66944119 | 66866052 | GGTGGTTTATAACAGATGGGACTTTTAGGC | |
| chrX: 66763874- | chrX: 66865693- | TGAAGGCAGGGGGCTGTCTTATTACCCAAAGAGGACATTTATTGATTTGGTTTTCTTTTTCCATTTTTACAATGCATCTTTATCGCCCAT | 907 |
| 66944119 | 66865812 | ATGGCCTTTCTGGAGGTGGTTTTCAGTCTG | |
| chrX: 66763874- | chrX: 66866293- | GTGTGTATGGGGAGAGAAAGTTGGTCAAAGTCTGTGTCTGAGTGCTTGGTGGGATGAACTCTGGGTTAGAAACAGGCATGGAGGGAAATA | 908 |
| 66944119 | 66866412 | GTTGGTTTATGGAGTGGGTAGGATGAGTGG | |
| chrX: 66763874- | chrX: 66866657- | TTTCAGCAAACTCACATGTATTTATACCTGCATAAGTTTTTGGTCTTGCTTTCCTAGAAGGTGACTAATCCCAGATCCTAATCAATTAAA | 909 |
| 66944119 | 66866776 | GAAGCAATCTTCAGATGGGGATAGAGCCAG | |
| chrX: 66763874- | chrX: 66866948- | GTGCATTATGTGCTCAAGAATTTACTATTTTTCAGACATTTTCTAGTAAAACATTGAAGATTATATGTCCATTTGTTTTGTACACATGGA | 910 |
| 66944119 | 66867067 | GTGCTGTTTGGTACACATCATAAAATTGAA | |
| chrX: 66763874- | chrX: 66867308- | AGCAGAGAGTTTTTGGTGCATATCAGTGTCCCCTTCACTCCCTGACTTTTCAAGTAACATTTCCCAGAGGCAAATTAACTCTGCTAAGAG | 911 |
| 66944119 | 66867427 | GATCTGCTTGCAGCTTCAACAGAGCCTTCA | |
| chrX: 66763874- | chrX: 66867068- | ACTGTAGTTTACATTCTGAACTCAAAGAATTACACCATCCTCACTGATGTTTACAATAGGTCCCAATTTAGTTTCTTTAGCAAATTTTAT | 912 |
| 66944119 | 66867187 | GTAAGTATGGCTTTGATTCTCTCTCTCACT | |
| chrX: 66763874- | chrX: 66867008- | TTATATGTCCATTTGTTTTGTACACATGGAGTGCTGTTTGGTACACATCATAAAATTGAAACTGTAGTTTACATTCTGAACTCAAAGAAT | 913 |
| 66944119 | 66867127 | TACACCATCCTCACTGATGTTTACAATAGG | |
| chrX: 66763874- | chrX: 66867428- | TCAGGTATCTTTGGCCAAGGAGTTGACTGATCCTGACTTTGCGAGTCCTAGAGATCTTTTCACAAAGCTCCTCTCATGTTTCTGCCTCTG | 914 |
| 66944119 | 66867547 | ATTTTCTTAAATGTCACAGACAGACTTTAG | |
| chrX: 66763874- | chrX: 66867188- | CCAGGTTTTTGTTAGGGAAGAAATGCAAGTGAACCCTCATTGAACTCTTTCTGTCCTTTAAATCCATTCTTTCCCACCTCAACTCATGTG | 915 |
| 66944119 | 66867307 | GAATTGAATGTTGCCTCTAGTTTGGAGTCT | |
| chrX: 66763874- | chrX: 66867368- | TTCCCAGAGGCAAATTAACTCTGCTAAGAGGATCTGCTTGCAGCTTCAACAGAGCCTTCATCAGGTATCTTTGGCCAAGGAGTTGACTGA | 916 |
| 66944119 | 66867487 | TCCTGACTTTGCGAGTCCTAGAGATCTTTT | |
| chrX: 66763874- | chrX: 66867248- | AATCCATTCTTTCCCACCTCAACTCATGTGGAATTGAATGTTGCCTCTAGTTTGGAGTCTAGCAGAGAGTTTTTGGTGCATATCAGTGTC | 917 |
| 66944119 | 66867367 | CCCTTCACTCCCTGACTTTTCAAGTAACAT | |
| chrX: 66763874- | chrX: 66867128- | TCCCAATTTAGTTTCTTTAGCAAATTTTATGTAAGTATGGCTTTGATTCTCTCTCTCACTCCAGGTTTTTGTTAGGGAAGAAATGCAAGT | 918 |
| 66944119 | 66867247 | GAACCCTCATTGAACTCTTTCTGTCCTTTA | |
| chrX: 66763874- | chrX: 66868597- | ATAGCACTTGCACCATTATGTAAATATCTGTAATGCTTACATAACTTTTGTCACTTGCAAGACCTTTTGAGTCCATTGCCTTCTGCTACC | 919 |
| 66944119 | 66868716 | ATGCCTTACCAATTTCCTAGTCCCTTATTA | |
| chrX: 66763874- | chrX: 66868417- | GGCTTGCAGAAGAAATAATTTAGCTCTGTAACTCATTGAAGTTGGTGCCCACCCAAGTCTCTGTCAGTGCCCAATTCGGGAGCCATGCCA | 920 |
| 66944119 | 66868536 | AGAATTTGCCATTGCTGCTTCATGGTGGCC | |
| chrX: 66763874- | chrX: 66868117- | CCAGAGGAGAAACTGAAAATATTTCAAACATTTTCTAGACTTCTGTGTTGTAAATTTGTGGATAACTATGAACTATATATGAATGAACTT | 921 |
| 66944119 | 66868236 | TTCTGGATGACACATATATTCCAGATGGTA | |
| chrX: 66763874- | chrX: 66868357- | TCTGTCTTCGCACACTCACCGGCTTAATTCTGGGCCTCCCCATAACACGACTAGACCACAGGCTTGCAGAAGAAATAATTTAGCTCTGTA | 922 |
| 66944119 | 66868476 | ACTCATTGAAGTTGGTGCCCACCCAAGTCT | |
| chrX: 66763874- | chrX: 66867997- | GTGGCTTCTTGGTCCTGAGCTTATTTACTAAACAAGAGAAAAAATAAATAAGTCTAGAAATGCTAGAAGAGGATACTTTTTTGTTTTAAT | 923 |
| 66944119 | 66868116 | GATCTAGTAGATCACTCCTCCTTGCAATAC | |
| chrX: 66763874- | chrX: 66868297- | TAGAAAGTAGATCCCAGGAGGCCAGCAGAGTTGTGGATCTGCCATATATTACCTCATGATTCTGTCTTCGCACACTCACCGGCTTAATTC | 924 |
| 66944119 | 66868416 | TGGGCCTCCCCATAACACGACTAGACCACA | |
| chrX: 66763874- | chrX: 66868057- | TGCTAGAAGAGGATACTTTTTTGTTTTAATGATCTAGTAGATCACTCCTCCTTGCAATACCCAGAGGAGAAACTGAAAATATTTCAAACA | 925 |
| 66944119 | 66868176 | TTTTCTAGACTTCTGTGTTGTAAATTTGTG | |
| chrX: 66763874- | chrX: 66867937- | TGGTATCTTAGTAGGCCAGTCAAAGTTTGAACAACTTGTTAGCACAGAATACCTGGCCTAGTGGCTTCTTGGTCCTGAGCTTATTTACTA | 926 |
| 66944119 | 66868056 | AACAAGAGAAAAAATAAATAAGTCTAGAAA | |
| chrX: 66763874- | chrX: 66867877- | GCTTTAGCTAATCACTTCTGTACTTATAAATCTGCATAGGTTTTATGTTTTTCCATCTCTTGGTATCTTAGTAGGCCAGTCAAAGTTTGA | 927 |
| 66944119 | 66867996 | ACAACTTGTTAGCACAGAATACCTGGCCTA | |
| chrX: 66763874- | chrX: 66868237- | AAAAGGAAGGGCTTTGGGGACTCTCTGGTACCAAGTGTCATGGAAAAACTGTGTGTCTCATAGAAAGTAGATCCCAGGAGGCCAGCAGAG | 928 |
| 66944119 | 66868356 | TTGTGGATCTGCCATATATTACCTCATGAT | |
| chrX: 66763874- | chrX: 66868477- | CTGTCAGTGCCCAATTCGGGAGCCATGCCAAGAATTTGCCATTGCTGCTTCATGGTGGCCTTGTGCCTGCTTATTTATAGCCTGTGCATT | 929 |
| 66944119 | 66868596 | TTATGAAACAGGGATTAATAAGAAGTTGCC | |
| chrX: 66763874- | chrX: 66868537- | TTGTGCCTGCTTATTTATAGCCTGTGCATTTTATGAAACAGGGATTAATAAGAAGTTGCCATAGCACTTGCACCATTATGTAAATATCTG | 930 |
| 66944119 | 66868656 | TAATGCTTACATAACTTTTGTCACTTGCAA | |
| chrX: 66763874- | chrX: 66868177- | GATAACTATGAACTATATATGAATGAACTTTTCTGGATGACACATATATTCCAGATGGTAAAAAGGAAGGGCTTTGGGGACTCTCTGGTA | 931 |
| 66944119 | 66868296 | CCAAGTGTCATGGAAAAACTGTGTGTCTCA | |
| chrX: 66763874- | chrX: 66870229- | TTCAGGGAATTGCTGGGTGACTATCAAACTCTGGTAGTTCATTTTTGCAGTTGGCTGCTGTTGTGAGGATAAGAGTTAGACTCACTTTCT | 932 |
| 66944119 | 66870348 | CTTCAGAGATAGAAATTATGTATTAATTCT | |
| chrX: 66763874- | chrX: 66870289- | TTGTGAGGATAAGAGTTAGACTCACTTTCTCTTCAGAGATAGAAATTATGTATTAATTCTCTGGGTTCTAGACCCACAGCAAGGAGCATA | 933 |
| 66944119 | 66870408 | CTGCTCCTCAAAATAACTGAATTCTGCGAG | |
| chrX: 66763874- | chrX: 66870409- | AAGCCATCATTGTAAAACAACAATATCTTCAGTTATAGTAGCCATGTGTGCAACTTCTGGAAACTGTTATTCAGATTTTCATGTTCCTTC | 934 |
| 66944119 | 66870528 | CCTGTCTCTTCATAGCTAGGCAGCTGCTTT | |
| chrX: 66763874- | chrX: 66870169- | accCTTATCAATTTTTTATGTGCCTCTCCATATTCTGCAGTCAGAAGCTTCTTCAGTCCTTTCAGGGAATTGCTGGGTGACTATCAAACT | 935 |
| 66944119 | 66870288 | CTGGTAGTTCATTTTTGCAGTTGGCTGCTG | |
| chrX: 66763874- | chrX: 66870469- | AAACTGTTATTCAGATTTTCATGTTCCTTCCCTGTCTCTTCATAGCTAGGCAGCTGCTTTCAGCCTTGTACAGATGCTAGTGAGCTTTCT | 936 |
| 66944119 | 66870588 | ACCTACAAACCTGCAGAAAATTGAACTGAG | |
| chrX: 66763874- | chrX: 66870649- | TCCCAGGCTGTGTTGTGACTACTGAGGCACTCCAGTGAAATCACTATTCCTCCTATCTAGACTAATGCCTGTCTCTGCAGAGCACCTCAT | 937 |
| 66944119 | 66870768 | AAGAACAGGCCTGGTAGTAATATCCTCatg | |
| chrX: 66763874- | chrX: 66870589- | ATTTGGAGGTGAAAGACTCTTGATAAAGGGAACAAGGTTTAGAATTCTCAGTCCCTTTGCTCCCAGGCTGTGTTGTGACTACTGAGGCAC | 938 |
| 66944119 | 66870708 | TCCAGTGAAATCACTATTCCTCCTATCTAG | |
| chrX: 66763874- | chrX: 66870349- | CTGGGTTCTAGACCCACAGCAAGGAGCATACTGCTCCTCAAAATAACTGAATTCTGCGAGAAGCCATCATTGTAAAACAACAATATCTTC | 939 |
| 66944119 | 66870468 | AGTTATAGTAGCCATGTGTGCAACTTCTGG | |
| chrX: 66763874- | chrX: 66870529- | CAGCCTTGTACAGATGCTAGTGAGCTTTCTACCTACAAACCTGCAGAAAATTGAACTGAGATTTGGAGGTGAAAGACTCTTGATAAAGGG | 940 |
| 66944119 | 66870648 | AACAAGGTTTAGAATTCTCAGTCCCTTTGC | |
| chrX: 66763874- | chrX: 66872149- | AACAATTCCTTAAGTTACATAAGCACATTCCTACAGGTCACAAGCTCATTTACTTACAGGATGGTTGATTTGGTCACAGGTTATTTCATG | 941 |
| 66944119 | 66872268 | AAAATACTTAAAAGATTTGCAGTGTTCAAA | |
| chrX: 66763874- | chrX: 66871849- | AAATGTAGATTAATGGTTCCTTGGCTCTTTGGTTTGAGCCTTCTCAGCAGAGCATCCCACGGAGTGTTTTCCATGGGGCCACGAGCAAGA | 942 |
| 66944119 | 66871968 | GAAATCCACTTCCCTCCTCCTCAATGTCAG | |
| chrX: 66763874- | chrX: 66871549- | TATCGCTAACATCACAGGGAACTTGTCTTCCTAAGAAAATTCCAAGCACTTAAAACCGCTGGTAGTTCATCAGCAACTCTCTTCATTAGA | 943 |
| 66944119 | 66871668 | TGTGCGAGGGACATGTGGGCCATAGTCCTT | |
| chrX: 66763874- | chrX: 66871669- | CTACTAACTTATATTCTTCAGGGGAAAGTTCTGATTCTGATGAGACCCAGCATGGTAGCTCTTAATTCACTGTTGTCACACGACTATAGA | 944 |
| 66944119 | 66871788 | ACAGGAAGCACAACTTAACACCTGTGCTCA | |
| chrX: 66763874- | chrX: 66871729- | CTTAATTCACTGTTGTCACACGACTATAGAACAGGAAGCACAACTTAACACCTGTGCTCATGAGAATTTTGCTCCTTATGACCAAGCTAA | 945 |
| 66944119 | 66871848 | AGAAAGAGCTTAGACAGGATGTGTGGCTAT | |
| chrX: 66763874- | chrX: 66871789- | TGAGAATTTTGCTCCTTATGACCAAGCTAAAGAAAGAGCTTAGACAGGATGTGTGGCTATAAATGTAGATTAATGGTTCCTTGGCTCTTT | 946 |
| 66944119 | 66871908 | GGTTTGAGCCTTCTCAGCAGAGCATCCCAC | |
| chrX: 66763874- | chrX: 66871969- | AAAATAGAGAATATTGTCTTTCAGGATAGAATTAAAAAGTCATAGAGGCAGCAACTTGTTTTCCTATATTAGGGTTTTAAAATTCTGTTT | 947 |
| 66944119 | 66872088 | TTCCTTCCTCTCCTGGGTCAGATCATTGTG | |
| chrX: 66763874- | chrX: 66872089- | TGGATGGACCTTGATTTCATTGTGGTATCTGTATGTGGACCCTGAAGACCATGGACTTCTAACAATTCCTTAAGTTACATAAGCACATTC | 948 |
| 66944119 | 66872208 | CTACAGGTCACAAGCTCATTTACTTACAGG | |
| chrX: 66763874- | chrX: 66872029- | TTCCTATATTAGGGTTTTAAAATTCTGTTTTTCCTTCCTCTCCTGGGTCAGATCATTGTGTGGATGGACCTTGATTTCATTGTGGTATCT | 949 |
| 66944119 | 66872148 | GTATGTGGACCCTGAAGACCATGGACTTCT | |
| chrX: 66763874- | chrX: 66871429- | AAGTACTATGCCTAGGCAACAAGAAAGGCAGCAATGAAGAGCAACAGCAGAGTCAAATATGAGAGAAGGAAGTTAAGAAAGATGTTAAGT | 950 |
| 66944119 | 66871548 | ACTGTGGGGAGTAACTGAGAAACCACCAAG | |
| chrX: 66763874- | chrX: 66871489- | GAGAGAAGGAAGTTAAGAAAGATGTTAAGTACTGTGGGGAGTAACTGAGAAACCACCAAGTATCGCTAACATCACAGGGAACTTGTCTTC | 951 |
| 66944119 | 66871608 | CTAAGAAAATTCCAAGCACTTAAAACCGCT | |
| chrX: 66763874- | chrX: 66871609- | GGTAGTTCATCAGCAACTCTCTTCATTAGATGTGCGAGGGACATGTGGGCCATAGTCCTTCTACTAACTTATATTCTTCAGGGGAAAGTT | 952 |
| 66944119 | 66871728 | CTGATTCTGATGAGACCCAGCATGGTAGCT | |
| chrX: 66763874- | chrX: 66871909- | GGAGTGTTTTCCATGGGGCCACGAGCAAGAGAAATCCACTTCCCTCCTCCTCAATGTCAGAAAATAGAGAATATTGTCTTTCAGGATAGA | 953 |
| 66944119 | 66872028 | ATTAAAAAGTCATAGAGGCAGCAACTTGTT | |
| chrX: 66763874- | chrX: 66872513- | AGAATTATGTGAGGCAGCATAGTAAGCATTTATGGCCCTTGGTTCCTAGAAGGAGCTTAGTCCCTGATAGTCATCTCTGCCTTTGCCATT | 954 |
| 66944119 | 66872632 | GTGTGAGACTGTCTTCTGTAACTGTATGTC | |
| chrX: 66763874- | chrX: 66872573- | TCCCTGATAGTCATCTCTGCCTTTGCCATTGTGTGAGACTGTCTTCTGTAACTGTATGTCTTCCTCCCTAGTAAGTTAATGAGTAATAAA | 955 |
| 66944119 | 66872692 | GGTATTCTATAGTGAGAGGACTCTGTAAGA | |
| chrX: 66763874- | chrX: 66872453- | GACTCTCCTGCCTAATAGCTAATTAGCAGAGTCACAGAGGTCATTACCTTGCAATTCTCAAGAATTATGTGAGGCAGCATAGTAAGCATT | 956 |
| 66944119 | 66872572 | TATGGCCCTTGGTTCCTAGAAGGAGCTTAG | |
| chrX: 66763874- | chrX: 66872817- | cagaaTACCAGTCTTGTCTTTGGTAAGGATTTTATAGACCCATCCTGACTACAGTGATATCCAACATGGCTATGTAATGACTGGCACTTT | 957 |
| 66944119 | 66872936 | CCCCACATAACATATATTTATTCCACactc | |
| chrX: 66763874- | chrX: 66873460- | GCAAGCTGTTTGACAGGCCTTCAGTTGGCTCTTTGTACCTTGCTCCCTCCGCATGCTGAGCTGTCCATAGCTGCCCTAGGCTGGTGTCTG | 958 |
| 66944119 | 66873579 | GGATTTTCGGAAGAAGGTTACTATCCAGGT | |
| chrX: 66763874- | chrX: 66874295- | CCTCTTTACTTTTTACCTCCCAGTACACTGTGAGTAACATTCCCCAGCCAGCCCAGCCAGCACGTGTTCATTGCCTCTCTTGACTTCCAG | 959 |
| 66944119 | 66874414 | ACTTTGGACTTGAAGGTGTCAGAGCTCTCT | |
| chrX: 66763874- | chrX: 66875375- | GATATGGAGGTATAGAGTGATTTCCCACCTACCTAGTGAGCACTACTGAGATATTCAAGTACTCTCTACCCAAGAATTCTATTGATATAA | 960 |
| 66944119 | 66875494 | AGGTAAAAAACTTGatcttaggactaatat | |
| chrX: 66763874- | chrX: 66875315- | GTTTAAGTTCAAATTATTGTTCCATGGGAGCAGAGATAGATATAGGAAACAAAAAAAAGGGATATGGAGGTATAGAGTGATTTCCCACCT | 961 |
| 66944119 | 66875434 | ACCTAGTGAGCACTACTGAGATATTCAAGT | |
| chrX: 66763874- | chrX: 66875075- | AGTAGACATCCTGTCCTTGAGGTTCCTTAACTCTGCTCAGCTTCAGAATACAGAAGGGGTTGGTTCTTCATTTGTGTTGTTTATAACTAA | 962 |
| 66944119 | 66875194 | AAGCCTCCTACTCCCCACTTTTTTGCATAG | |
| chrX: 66763874- | chrX: 66874475- | ACTGTCCAGGAGAAAAGCTAGCAAGGTCATAAATTATTCTCCATATTTTCCAGCCATTGGTTTCCCTTGTCCAGCCAGAGGTGTGTCTCA | 963 |
| 66944119 | 66874594 | AAGTATGCTGAGGCCAGATTCAATAGAAAC | |
| chrX: 66763874- | chrX: 66875255- | ATGTCACTTGGAAAGAGTTTTCTTTGTCTCTTTTGCAACTTGACAATGACTAGCCAGCAAGTTTAAGTTCAAATTATTGTTCCATGGGAG | 964 |
| 66944119 | 66875374 | CAGAGATAGATATAGGAAACAAAAAAAAGG | |
| chrX: 66763874- | chrX: 66874415- | GTGTATCTTTGTCCCCAACAAGATAAGTCTGACCTCCCCAGCAAATTCAAGTCCTAAGCCACTGTCCAGGAGAAAAGCTAGCAAGGTCAT | 965 |
| 66944119 | 66874534 | AAATTATTCTCCATATTTTCCAGCCATTGG | |
| chrX: 66763874- | chrX: 66875015- | AAGCTCCATCTGAAGAGAGGGGAATAACACCCAGCCAAGAGCCCTCAGGGCCCATCAGTAAGTAGACATCCTGTCCTTGAGGTTCCTTAA | 966 |
| 66944119 | 66875134 | CTCTGCTCAGCTTCAGAATACAGAAGGGGT | |
| chrX: 66763874- | chrX: 66874055- | tactggtactcagtaagTTTTGTATCCTTTTCCTAGAGTGAGTCTTGGTCATAGGCATGCGTATACTTGCAGCGTCCCTGGGTAGGCCGA | 967 |
| 66944119 | 66874174 | AAGAGCAAATAAGAGATGGTATCTATGGTA | |
| chrX: 66763874- | chrX: 66874895- | ACATCCCTCTGTCTTCACCCACTCTTCAGTTCTGATTCTTTTAAAAGCAGCCAACCAAAACCAGCAAGTACATACTGCTTATCTCTGACT | 968 |
| 66944119 | 66875014 | TCCACCAGAATCAACTTCAGATCTTGTCCA | |
| chrX: 66763874- | chrX: 66875135- | TGGTTCTTCATTTGTGTTGTTTATAACTAAAAGCCTCCTACTCCCCACTTTTTTGCATAGCTTCTTCTGCCATCCCACCTGTGTAGCCTC | 969 |
| 66944119 | 66875254 | TTCAACTCCCCCAAAACTCCTCTGTAGCCC | |
| chrX: 66763874- | chrX: 66875195- | CTTCTTCTGCCATCCCACCTGTGTAGCCTCTTCAACTCCCCCAAAACTCCTCTGTAGCCCATGTCACTTGGAAAGAGTTTTCTTTGTCTC | 970 |
| 66944119 | 66875314 | TTTTGCAACTTGACAATGACTAGCCAGCAA | |
| chrX: 66763874- | chrX: 66874175- | TTCCCCAGGTAAAGGAGGCCTTGGGTTGGCATAAGATTTCACTTCTCTTTAGAGTTACTTAATTAGGGACCAGAAAGGCCATCAGCATTT | 971 |
| 66944119 | 66874294 | GTATGAGAATATAACAAAGGTCAATCTCTT | |
| chrX: 66763874- | chrX: 66874595- | CTGAGCCAGCACCTGTGTAAATAATTTTTAAAGCTCCTTTTCCTGAAGCTGGATGAATATTTTTAAAAACTAAGCTGGATTGTCTTTTAT | 972 |
| 66944119 | 66874714 | CTAGCATGCCGTCTCCTACATTCCTAGTGC | |
| chrX: 66763874- | chrX: 66874775- | GAGGGAGACATTTCTTTCAGAAGCAAGGTAATACTTTGGTCTGGTCTATGACTCTATTTTGTTTAAAATGAAACTATGGCAGTATAGTGG | 973 |
| 66944119 | 66874894 | TATTCATTCTGCTTCCCATAGGTTAACTTT | |
| chrX: 66763874- | chrX: 66874955- | CCAGCAAGTACATACTGCTTATCTCTGACTTCCACCAGAATCAACTTCAGATCTTGTCCAAAGCTCCATCTGAAGAGAGGGGAATAACAC | 974 |
| 66944119 | 66875074 | CCAGCCAAGAGCCCTCAGGGCCCATCAGTA | |
| chrX: 66763874- | chrX: 66874535- | TTTCCCTTGTCCAGCCAGAGGTGTGTCTCAAAGTATGCTGAGGCCAGATTCAATAGAAACCTGAGCCAGCACCTGTGTAAATAATTTTTA | 975 |
| 66944119 | 66874654 | AAGCTCCTTTTCCTGAAGCTGGATGAATAT | |
| chrX: 66763874- | chrX: 66874835- | GTTTAAAATGAAACTATGGCAGTATAGTGGTATTCATTCTGCTTCCCATAGGTTAACTTTACATCCCTCTGTCTTCACCCACTCTTCAGT | 976 |
| 66944119 | 66874954 | TCTGATTCTTTTAAAAGCAGCCAACCAAAA | |
| chrX: 66763874- | chrX: 66874235- | AATTAGGGACCAGAAAGGCCATCAGCATTTGTATGAGAATATAACAAAGGTCAATCTCTTCCTCTTTACTTTTTACCTCCCAGTACACTG | 977 |
| 66944119 | 66874354 | TGAGTAACATTCCCCAGCCAGCCCAGCCAG | |
| chrX: 66763874- | chrX: 66874715- | TATGGACCTCTTGGAGGAATGTGGTTTGGTTATAGTGGTATTGTCTTGTCTGTTGTGGGGGAGGGAGACATTTCTTTCAGAAGCAAGGTA | 978 |
| 66944119 | 66874834 | ATACTTTGGTCTGGTCTATGACTCTATTTT | |
| chrX: 66763874- | chrX: 66874355- | CACGTGTTCATTGCCTCTCTTGACTTCCAGACTTTGGACTTGAAGGTGTCAGAGCTCTCTGTGTATCTTTGTCCCCAACAAGATAAGTCT | 979 |
| 66944119 | 66874474 | GACCTCCCCAGCAAATTCAAGTCCTAAGCC | |
| chrX: 66763874- | chrX: 66874115- | GTATACTTGCAGCGTCCCTGGGTAGGCCGAAAGAGCAAATAAGAGATGGTATCTATGGTATTCCCCAGGTAAAGGAGGCCTTGGGTTGGC | 980 |
| 66944119 | 66874234 | ATAAGATTTCACTTCTCTTTAGAGTTACTT | |
| chrX: 66763874- | chrX: 66874655- | TTTTAAAAACTAAGCTGGATTGTCTTTTATCTAGCATGCCGTCTCCTACATTCCTAGTGCTATGGACCTCTTGGAGGAATGTGGTTTGGT | 981 |
| 66944119 | 66874774 | TATAGTGGTATTGTCTTGTCTGTTGTGGGG | |
| chrX: 66763874- | chrX: 66875781- | TAATTTGTCTGTTGCTATTTATACGGATAATTTGATAGTAGTTATTTTTGGACATGGATAGCTTTGAAGCCTTACAGATGAGTCCATCCC | 982 |
| 66944119 | 66875900 | CAAGTACCCAAAACTAAAGAAAGTTGGCTA | |
| chrX: 66763874- | chrX: 66875841- | GCTTTGAAGCCTTACAGATGAGTCCATCCCCAAGTACCCAAAACTAAAGAAAGTTGGCTAGAGTGATGACAAGGTGGCAGCACAGAGCTC | 983 |
| 66944119 | 66875960 | CCTGCGTTCTGGGCCCTGTCCCCTAGCTAG | |
| chrX: 66763874- | chrX: 66875721- | ATTACAGCACATTTTCAATGCTCCAATTATGTCACTGTAGAAATGCTAATGTGGATTAAATAATTTGTCTGTTGCTATTTATACGGATAA | 984 |
| 66944119 | 66875840 | TTTGATAGTAGTTATTTTTGGACATGGATA | |
| chrX: 66763874- | chrX: 66875901- | GAGTGATGACAAGGTGGCAGCACAGAGCTCCCTGCGTTCTGGGCCCTGTCCCCTAGCTAGAGAGAACTCCAGGCTATAAGCATTTGTATT | 985 |
| 66944119 | 66876020 | CTCATAGTCCAATGGCAGGGAAGAAGGGCT | |
| chrX: 66763874- | chrX: 66875661- | GAATCAAACACTGATAACAGTACTTCCTAGTACACAAATGAGAAATCAGTCCCTCATCAAATTACAGCACATTTTCAATGCTCCAATTAT | 986 |
| 66944119 | 66875780 | GTCACTGTAGAAATGCTAATGTGGATTAAA | |
| chrX: 66763874- | chrX: 66876133- | ctggaggGCCTGCCTTCCAAGGTCAGTTGCAGCTTTAGCACTATAAAGAGCACCTACCTGCGGCAGATACAATGTGATGGGACATGACAG | 987 |
| 66944119 | 66876252 | AGAAAAAATCTATAAGCAGAGCCTCCCCAT | |
| chrX: 66763874- | chrX: 66876253- | TCCCAGGCATTGAAACAATCCTAACCAAGACTGGCATAGTACAATGAGCCTGTCCCTATCAGCAGGTTTGGAAGCCTTAACAACAACAAC | 988 |
| 66944119 | 66876372 | AAAAACAATAATAATGGTGATGATaatcat | |
| chrX: 66763874- | chrX: 66876193- | CGGCAGATACAATGTGATGGGACATGACAGAGAAAAAATCTATAAGCAGAGCCTCCCCATTCCCAGGCATTGAAACAATCCTAACCAAGA | 989 |
| 66944119 | 66876312 | CTGGCATAGTACAATGAGCCTGTCCCTATC | |
| chrX: 66763874- | chrX: 66876955- | TCAATTTGGTTGATAATTTGGTTGAAATGAATTTATTTCATTTTTTATTCCATCCTTACAATGGAAGATTAGTGCTTGTTTCCCACCCAA | 990 |
| 66944119 | 66877074 | GGATACCAGGATATTTCAGGGGCTGTATTA | |
| chrX: 66763874- | chrX: 66876895- | TTATAAAACAACTAATTGATTTAAATCAAAGCCCAAACAGAAGTGTTTGCTAATTTTATTTCAATTTGGTTGATAATTTGGTTGAAATGA | 991 |
| 66944119 | 66877014 | ATTTATTTCATTTTTTATTCCATCCTTACA | |
| chrX: 66763874- | chrX: 66876835- | GAGATCACTGTAGCAATTGATTGGTTTAAATCAAAGCCCCCAAAAAAATGTTATTGAGAATTATAAAACAACTAATTGATTTAAATCAAA | 992 |
| 66944119 | 66876954 | GCCCAAACAGAAGTGTTTGCTAATTTTATT | |
| chrX: 66763874- | chrX: 66877714- | TTTTTAAATTTCCTTAGCATCCATTTCCACCATTGGAAATTCAGGGTCAAAACAGGGGTTTGGGATTGGAGCATGTCTATCACAGATAAC | 993 |
| 66944119 | 66877833 | CAATCATGTGTTATGACTTAAGAATTTATG | |
| chrX: 66763874- | chrX: 66877594- | CCCTCATGCATGCATATAATTTATAGAGTCACTGTTTTGCTCGGTTGTCCTCATGCCTCTATATTATTGGAGGTTTAGATTGTTTCCATA | 994 |
| 66944119 | 66877713 | TACTCAGGTTGTATTCATGTCCTTTTTTTC | |
| chrX: 66763874- | chrX: 66877654- | ATATTATTGGAGGTTTAGATTGTTTCCATATACTCAGGTTGTATTCATGTCCTTTTTTTCTTTTTAAATTTCCTTAGCATCCATTTCCAC | 995 |
| 66944119 | 66877773 | CATTGGAAATTCAGGGTCAAAACAGGGGTT | |
| chrX: 66763874- | chrX: 66877534- | AAGCCCACCACCCTAACACAACAAATACTTAAAACTTGTCTTCATTTCCTTTAGGTCTGGCCCTCATGCATGCATATAATTTATAGAGTC | 996 |
| 66944119 | 66877653 | ACTGTTTTGCTCGGTTGTCCTCATGCCTCT | |
| chrX: 66763874- | chrX: 66877834- | AAAGGGCCCTCTACCTGAAGATATCTTGCTACTGATGCTGTCTCACAGTGTCTGAAACTCCCATCATATGTGGAATTGTTTTGGAAGGCT | 997 |
| 66944119 | 66877953 | TTGCCTCCTGGGACACATTCAGCCATAATC | |
| chrX: 66763874- | chrX: 66877954- | AAGAAATAGTATTGAGCATTAGACTGTCAGTATGTCCATTAGCAAGACTGTGGAGGAATGGAATCACCAATATTATATTTTATAGGGGAT | 998 |
| 66944119 | 66878073 | ACAGAATACAAGAGAAGTTCTGAAGAGAAA | |
| chrX: 66763874- | chrX: 66877774- | TGGGATTGGAGCATGTCTATCACAGATAACCAATCATGTGTTATGACTTAAGAATTTATGAAAGGGCCCTCTACCTGAAGATATCTTGCT | 999 |
| 66944119 | 66877893 | ACTGATGCTGTCTCACAGTGTCTGAAACTC | |
| chrX: 66763874- | chrX: 66878074- | ATTCTTATGTAGAATAGGAAGGCTTAGATACAGCATGAAAGCTGCAGGCTTTGAGGAGCCAGAGGTCAAATGAAAGCATTGAGTATTTGT | 1000 |
| 66944119 | 66878193 | TTAGATGAAAGAACAGAAAGGGAAAAAGAA | |
| chrX: 66763874- | chrX: 66878134- | AGAGGTCAAATGAAAGCATTGAGTATTTGTTTAGATGAAAGAACAGAAAGGGAAAAAGAAGCAGAGGAAGGGATAGTAGAGAGAAATGTA | 1001 |
| 66944119 | 66878253 | TAAGTTTTATCCATTTAACTTGTAATTGTG | |
| chrX: 66763874- | chrX: 66878014- | GAATCACCAATATTATATTTTATAGGGGATACAGAATACAAGAGAAGTTCTGAAGAGAAAATTCTTATGTAGAATAGGAAGGCTTAGATA | 1002 |
| 66944119 | 66878133 | CAGCATGAAAGCTGCAGGCTTTGAGGAGCC | |
| chrX: 66763874- | chrX: 66877414- | CACATTGAAAACCTCTACTGGAGTGCATTGTGTCTGGTGGGCTTCAACCTTAATTCTTAAGTATGTGAAAACACATCACCTATCTGGAGG | 1003 |
| 66944119 | 66877533 | TTTACACTTTCTGCTAATGACTTTATTTTT | |
| chrX: 66763874- | chrX: 66877474- | GTATGTGAAAACACATCACCTATCTGGAGGTTTACACTTTCTGCTAATGACTTTATTTTTAAGCCCACCACCCTAACACAACAAATACTT | 1004 |
| 66944119 | 66877593 | AAAACTTGTCTTCATTTCCTTTAGGTCTGG | |
| chrX: 66763874- | chrX: 66877894- | CCATCATATGTGGAATTGTTTTGGAAGGCTTTGCCTCCTGGGACACATTCAGCCATAATCAAGAAATAGTATTGAGCATTAGACTGTCAG | 1005 |
| 66944119 | 66878013 | TATGTCCATTAGCAAGACTGTGGAGGAATG | |
| chrX: 66763874- | chrX: 66878885- | GACTCCAACATCATTACAGAACTATAAATTACATGTGGAAAAGAAAGGCCTCCTATGTTAGAATAGAAAATAAAATGCTGTGGGGTTGAG | 1006 |
| 66944119 | 66879004 | GGACAGAGGTGCTGTCTAGGAAGTCAGATA | |
| chrX: 66763874- | chrX: 66878825- | TCACTGAGATGGTTTGGGGATTGTGGCTTCCAGATGATCAGATTTTCTTTTTTAGGTAGAGACTCCAACATCATTACAGAACTATAAATT | 1007 |
| 66944119 | 66878944 | ACATGTGGAAAAGAAAGGCCTCCTATGTTA | |
| chrX: 66763874- | chrX: 66878765- | AGTAGGTGTATATGGGGAATTGGAGGGAGATAGGTGGCTGTGTTTAGTAATTGGTTGACTTCACTGAGATGGTTTGGGGATTGTGGCTTC | 1008 |
| 66944119 | 66878884 | CAGATGATCAGATTTTCTTTTTTAGGTAGA | |
| chrX: 66763874- | chrX: 66878525- | CATAGAACTGTAGGGGTCAAGGCCAAAGGGGACGTCCTGTTCCAAGTCACCTTCTTTGGACATTAGAAAACCACGAGGGGTTTGGAAATC | 1009 |
| 66944119 | 66878644 | AGAAAACCAGCAGAGGCAGGAAAACTCAGG | |
| chrX: 66763874- | chrX: 66878705- | ACTGCTGATGTGGAGTAGAGGCAGCTTTGTCTGCTGTGTGATAACCAAACCTTTACGAATAGTAGGTGTATATGGGGAATTGGAGGGAGA | 1010 |
| 66944119 | 66878824 | TAGGTGGCTGTGTTTAGTAATTGGTTGACT | |
| chrX: 66763874- | chrX: 66878585- | CATTAGAAAACCACGAGGGGTTTGGAAATCAGAAAACCAGCAGAGGCAGGAAAACTCAGGGCAGCATGGGAGATTCAGTATATACAAAAA | 1011 |
| 66944119 | 66878704 | GGTTCACACCAGTAATCAAACAGAATTTTA | |
| chrX: 66763874- | chrX: 66878465- | TGAGATCACTTGAAACTAGGGAGAGATGTGTGAGTTCTGGGCAACCAGTAGTTGGCTTTACATAGAACTGTAGGGGTCAAGGCCAAAGGG | 1012 |
| 66944119 | 66878584 | GACGTCCTGTTCCAAGTCACCTTCTTTGGA | |
| chrX: 66763874- | chrX: 66878945- | GAATAGAAAATAAAATGCTGTGGGGTTGAGGGACAGAGGTGCTGTCTAGGAAGTCAGATAGCGTTTTCCAGTTCTGTCCCTCAGAGTTCC | 1013 |
| 66944119 | 66879064 | TTGTCCTCATTGAGACTCAATTTCTCTTAC | |
| chrX: 66763874- | chrX: 66878645- | GCAGCATGGGAGATTCAGTATATACAAAAAGGTTCACACCAGTAATCAAACAGAATTTTAACTGCTGATGTGGAGTAGAGGCAGCTTTGT | 1014 |
| 66944119 | 66878764 | CTGCTGTGTGATAACCAAACCTTTACGAAT | |
| chrX: 66763874- | chrX: 66885609- | AGTATGTTGCCTCAAGGAGGCCCTCACTGTTCTAGGAAATATAATTCCAGAGTTTGCTGACTCACACCATGGAATATATGCATAAAATGG | 1015 |
| 66944119 | 66885728 | ATCCTGCAGATAAGCCTTTCTCTGACTAGT | |
| chrX: 66763874- | chrX: 66885489- | TAATAACACAAAGATCACTCTCGACTAGCAAGCCCTTTTATGATGGTGTGAGCATTTGACACCCTTGTTGCTAGTAACATCAGTGAGTGA | 1016 |
| 66944119 | 66885608 | CCTGACCCATTTTTGGAACAGAATATGATC | |
| chrX: 66763874- | chrX: 66885309- | AAAAAAACAATGAAGAGCTTTCTTTGGGCATTAGACACTTTCCCATAAGGTGGCTGACTCTCTTTTAGTCATGTCAGCTTGGCCCAATCT | 1017 |
| 66944119 | 66885428 | TCACTTGGTAGCCCTTCTTTCTTCTTCATT | |
| chrX: 66763874- | chrX: 66885249- | GAACAAACTAGAAATCCAGTATAGAAAATAAAAATAGGATTATATTCCTTGGAATCTCAGAAAAAAACAATGAAGAGCTTTCTTTGGGCA | 1018 |
| 66944119 | 66885368 | TTAGACACTTTCCCATAAGGTGGCTGACTC | |
| chrX: 66763874- | chrX: 66885129- | CCCACTGTTCTTTCTCATCTCAACCCTGAGTATAAGTACAGATCACATTCCTTGGGTTCTTAGAAAATAATAGAAATGAACTCTCATTCA | 1019 |
| 66944119 | 66885248 | TCAAAATGCCCATTAGTAAATACTGAGGGA | |
| chrX: 66763874- | chrX: 66885369- | TCTTTTAGTCATGTCAGCTTGGCCCAATCTTCACTTGGTAGCCCTTCTTTCTTCTTCATTAATCCATCTCCTATGCTCCTATGGGGTCCT | 1020 |
| 66944119 | 66885488 | AGAGAAATGCCCATCATGTACACACACATC | |
| chrX: 66763874- | chrX: 66885549- | ACCCTTGTTGCTAGTAACATCAGTGAGTGACCTGACCCATTTTTGGAACAGAATATGATCAGTATGTTGCCTCAAGGAGGCCCTCACTGT | 1021 |
| 66944119 | 66885668 | TCTAGGAAATATAATTCCAGAGTTTGCTGA | |
| chrX: 66763874- | chrX: 66885429- | AATCCATCTCCTATGCTCCTATGGGGTCCTAGAGAAATGCCCATCATGTACACACACATCTAATAACACAAAGATCACTCTCGACTAGCA | 1022 |
| 66944119 | 66885548 | AGCCCTTTTATGATGGTGTGAGCATTTGAC | |
| chrX: 66763874- | chrX: 66885189- | TAGAAAATAATAGAAATGAACTCTCATTCATCAAAATGCCCATTAGTAAATACTGAGGGAGAACAAACTAGAAATCCAGTATAGAAAATA | 1023 |
| 66944119 | 66885308 | AAAATAGGATTATATTCCTTGGAATCTCAG | |
| chrX: 66763874- | chrX: 66896872- | CTGGCCTCCCTTGACCCATTCCATTCATTATCTAAGGGACTCCAAGCCAGCATTCCACAGAGTGCCCTCACCAAACTCACTAAGACTGAA | 1024 |
| 66944119 | 66896991 | GGCGAACCAGGATTCCAAACAGCCATTATG | |
| chrX: 66763874- | chrX: 66897052- | TCCATACAGATACTACTATTCTTTAGGAAAACGTTAAAATCACATGATCTTCCAGGACCTGGGCTGCTTCTTTAAGAAGCATGTTACAGA | 1025 |
| 66944119 | 66897171 | AAGCTTTATTGGCCAACAACATATTGAAAG | |
| chrX: 66763874- | chrX: 66896572- | CAACTTGAGGAAGAGAGGTGATATTATCGGAATGAATTTCTTTGTTGTAAGTTATAAATGTATGGGCTTTTCCAATCCCATCACCCTTAA | 1026 |
| 66944119 | 66896691 | AACTTTATTTGTTTTCTGCAGTGAGGGTGT | |
| chrX: 66763874- | chrX: 66896512- | CAGCAGACAGGATTTGGATCTGGCATTTGGTAACAGGGCAGTTTCCAAAGTTGCTGTACGCAACTTGAGGAAGAGAGGTGATATTATCGG | 1027 |
| 66944119 | 66896631 | AATGAATTTCTTTGTTGTAAGTTATAAATG | |
| chrX: 66763874- | chrX: 66896332- | CCCAACAAGTGATCAGTAGTCAGAAAATGGCCAAGAAATACCATGGGGTGTGCCTTCCCATAACAGCTTATCTTTGTGTTTTAGTTGCAA | 1028 |
| 66944119 | 66896451 | GGTTACTAAAAGCCTGTGCAGGGTTTATGG | |
| chrX: 66763874- | chrX: 66896992- | AAAGGAAAGAGAGAGAGACTTAGGGTTTGCAAAATAAGATACCCTGTTGATTCTTTTTATTCCATACAGATACTACTATTCTTTAGGAAA | 1029 |
| 66944119 | 66897111 | ACGTTAAAATCACATGATCTTCCAGGACCT | |
| chrX: 66763874- | chrX: 66896392- | TAACAGCTTATCTTTGTGTTTTAGTTGCAAGGTTACTAAAAGCCTGTGCAGGGTTTATGGCAAAAGTAAAACTTGCTCCAGGAGCAAGCC | 1030 |
| 66944119 | 66896511 | CTTGTTTCATTGTCTAATGTTCTTAATCCC | |
| chrX: 66763874- | chrX: 66896752- | ATGTGGACTCTCTGAAATTGTTATAAGGTCTTTTTCTTTGTTTTTTTCTTGATGCCCAAGCTGCCAAGGGTAGTACTGGCAGTGGTGGGC | 1031 |
| 66944119 | 66896871 | AGACAAGGAGGTGATAGCAAACTTTGTCCT | |
| chrX: 66763874- | chrX: 66896452- | CAAAAGTAAAACTTGCTCCAGGAGCAAGCCCTTGTTTCATTGTCTAATGTTCTTAATCCCCAGCAGACAGGATTTGGATCTGGCATTTGG | 1032 |
| 66944119 | 66896571 | TAACAGGGCAGTTTCCAAAGTTGCTGTACG | |
| chrX: 66763874- | chrX: 66896632- | TATGGGCTTTTCCAATCCCATCACCCTTAAAACTTTATTTGTTTTCTGCAGTGAGGGTGTCTCCGTTGTCTTTAATATGCTTGCTTTGAG | 1033 |
| 66944119 | 66896751 | TTCATGGATGAACATTCCTGCCTGGCTGAC | |
| chrX: 66763874- | chrX: 66896932- | AGTGCCCTCACCAAACTCACTAAGACTGAAGGCGAACCAGGATTCCAAACAGCCATTATGAAAGGAAAGAGAGAGAGACTTAGGGTTTGC | 1034 |
| 66944119 | 66897051 | AAAATAAGATACCCTGTTGATTCTTTTTAT | |
| chrX: 66763874- | chrX: 66896692- | CTCCGTTGTCTTTAATATGCTTGCTTTGAGTTCATGGATGAACATTCCTGCCTGGCTGACATGTGGACTCTCTGAAATTGTTATAAGGTC | 1035 |
| 66944119 | 66896811 | TTTTTCTTTGTTTTTTTCTTGATGCCCAAG | |
| chrX: 66763874- | chrX: 66896812- | CTGCCAAGGGTAGTACTGGCAGTGGTGGGCAGACAAGGAGGTGATAGCAAACTTTGTCCTCTGGCCTCCCTTGACCCATTCCATTCATTA | 1036 |
| 66944119 | 66896931 | TCTAAGGGACTCCAAGCCAGCATTCCACAG | |
| chrX: 66763874- | chrX: 66897344- | CTGAATATCTTTACATGGTAATAACACAATGGAAAGCTTGCAAAATAGACAGAGGCTAGGGGAAGAAGGATTGAGTGTGAATATAGCCTC | 1037 |
| 66944119 | 66897463 | TTATAAATCGAGAGGAATGGTCTGTGTCTT | |
| chrX: 66763874- | chrX: 66898078- | AGTTTCCTATGCCAACCCCAGGCAGATTACATTTAATTTTATCTGATTTATATAGAGAGTTTCTATGTAATGTTTTATTCTTAAAAATAG | 1038 |
| 66944119 | 66898197 | TTTACTATAAAAAACTCAACTGgtttgatt | |
| chrX: 66763874- | chrX: 66898018- | CCACAACGCTAAGCCTGCATGGAAGAGGAGAAAAAGAGTGGCCTGACAAGAGAAGTTCCCAGTTTCCTATGCCAACCCCAGGCAGATTAC | 1039 |
| 66944119 | 66898137 | ATTTAATTTTATCTGATTTATATAGAGAGT | |
| chrX: 66763874- | chrX: 66897958- | tctaaaatGTTCCACAAAGCCCTGGGGTGGGGGGCTCCTACAGAGTTTCGCTAAGGCAAACCACAACGCTAAGCCTGCATGGAAGAGGAG | 1040 |
| 66944119 | 66898077 | AAAAAGAGTGGCCTGACAAGAGAAGTTCCC | |
| chrX: 66763874- | chrX: 66899048- | GGCCTTAAGTATTAAAGTATTAAGTATTAAAGTGATATGTAACCAAGTATATTGTTTGGTAACTTCATTTTTGTTATTATTTTAACAAAC | 1041 |
| 66944119 | 66899167 | CAATATATTGTGAATATACTTCCAAGTGAA | |
| chrX: 66763874- | chrX: 66900507- | GTGATGCCACTAATAGTGGGTAACTTCTCTGCCTTACCTCCTCTGTTCCAAACAGGATTTTTCAGAATGAACAAATTAAAAGAATCATAA | 1042 |
| 66944119 | 66900626 | TCAGACACTAACCCCAAGCCATACTGCATG | |
| chrX: 66763874- | chrX: 66900387- | gttcatataggacctgcaTTAACACCATATATATAAAGTGTGAGAAATACTACATTCTTCAGGATTCTCTGTAGGTTAACAATGAAGATG | 1043 |
| 66944119 | 66900506 | ATGACTCAACCCTTTCTTTGTTTGCATAAT | |
| chrX: 66763874- | chrX: 66900747- | ACTATGAAGAAAGACACAGAGCAACATCAGACAGTCAAGAATTTCAGAGCCAGCTGGCATGCAGTGGACCTCATGCCAGCCCATTTTATG | 1044 |
| 66944119 | 66900866 | ACTATTTAGGTAGTCAAGGGTTTAAGATTT | |
| chrX: 66763874- | chrX: 66900867- | TTCTAATAAGACAGTTATTATGCATTTCAATGAGTGATTTCTTTGCAGCTCTAGAGTGTGGCCTTACCTACTTCAACATGAGAAGATTTT | 1045 |
| 66944119 | 66900986 | TGTATTTTGTCAGTCATTTCACAATGACTT | |
| chrX: 66763874- | chrX: 66900567- | TTCAGAATGAACAAATTAAAAGAATCATAATCAGACACTAACCCCAAGCCATACTGCATGGCAGCACCAATGGGACTGACAGAAAACAAC | 1046 |
| 66944119 | 66900686 | AGAAATAGGAAGAAATCCTACAGAGAAACA | |
| chrX: 66763874- | chrX: 66901167- | CAGGATTACATCCCATTTACACAGTTCTCTGTCACTTGAATACAGAGAAGGGATCCACAAGGCCATATGCTTCCTAGACAAAGAGAAAAG | 1047 |
| 66944119 | 66901286 | ATTTCTGCCACACTCAGAACGCTTTGTCTT | |
| chrX: 66763874- | chrX: 66900687- | AACTTGAAAGCTGTCTCATGGCCTTTGAATCATACTTAAGTTTTATGATGGAAGGATACGACTATGAAGAAAGACACAGAGCAACATCAG | 1048 |
| 66944119 | 66900806 | ACAGTCAAGAATTTCAGAGCCAGCTGGCAT | |
| chrX: 66763874- | chrX: 66900807- | GCAGTGGACCTCATGCCAGCCCATTTTATGACTATTTAGGTAGTCAAGGGTTTAAGATTTTTCTAATAAGACAGTTATTATGCATTTCAA | 1049 |
| 66944119 | 66900926 | TGAGTGATTTCTTTGCAGCTCTAGAGTGTG | |
| chrX: 66763874- | chrX: 66900447- | AGGATTCTCTGTAGGTTAACAATGAAGATGATGACTCAACCCTTTCTTTGTTTGCATAATGTGATGCCACTAATAGTGGGTAACTTCTCT | 1050 |
| 66944119 | 66900566 | GCCTTACCTCCTCTGTTCCAAACAGGATTT | |
| chrX: 66763874- | chrX: 66901107- | TCCCTGTGTTAATTTTTCAGTCTCTTAGGTTATAGAGGACCTTCTAGAACCACCTTACAGCAGGATTACATCCCATTTACACAGTTCTCT | 1051 |
| 66944119 | 66901226 | GTCACTTGAATACAGAGAAGGGATCCACAA | |
| chrX: 66763874- | chrX: 66900927- | GCCTTACCTACTTCAACATGAGAAGATTTTTGTATTTTGTCAGTCATTTCACAATGACTTTTAGTGAGCCCTTCATTATAGACTGTGGAT | 1052 |
| 66944119 | 66901046 | ACAACTTTGCTGTTGGAAATTAACAGTGTC | |
| chrX: 66763874- | chrX: 66901047- | AAACAACTGGGTATAATGTTTGTAATATCTGAGGAGGGGGAGCTGCCTAGGAAGTTGTATTCCCTGTGTTAATTTTTCAGTCTCTTAGGT | 1053 |
| 66944119 | 66901166 | TATAGAGGACCTTCTAGAACCACCTTACAG | |
| chrX: 66763874- | chrX: 66901227- | GGCCATATGCTTCCTAGACAAAGAGAAAAGATTTCTGCCACACTCAGAACGCTTTGTCTTCAGACTATAATCACCCACACCATATTTCCT | 1054 |
| 66944119 | 66901346 | TTGGATCCACTTTccagatttttgtgctgg | |
| chrX: 66763874- | chrX: 66900987- | TTAGTGAGCCCTTCATTATAGACTGTGGATACAACTTTGCTGTTGGAAATTAACAGTGTCAAACAACTGGGTATAATGTTTGTAATATCT | 1055 |
| 66944119 | 66901106 | GAGGAGGGGGAGCTGCCTAGGAAGTTGTAT | |
| chrX: 66763874- | chrX: 66900627- | GCAGCACCAATGGGACTGACAGAAAACAACAGAAATAGGAAGAAATCCTACAGAGAAACAAACTTGAAAGCTGTCTCATGGCCTTTGAAT | 1056 |
| 66944119 | 66900746 | CATACTTAAGTTTTATGATGGAAGGATACG | |
| chrX: 66763874- | chrX: 66902710- | TTAGGACTTACAGAAAAAAGATTCCTTTCATATCCATCTTTGCAATCCTCAACCACTTCTGTCACTATTATGTGTCATTTCAAACATTAA | 1057 |
| 66944119 | 66902829 | ATTCCTCATTCTGCTTTGAAGGAACACATG | |
| chrX: 66763874- | chrX: 66902470- | gagagtgattattgttactGTTAAGAACTCTGATAGCCTCATCCATATATTTGGAGAAATTGAATAAATAATAGGAAAGAAATAATAGCA | 1058 |
| 66944119 | 66902589 | TCCCAATGATTTTACCTTGGCTCTACCATC | |
| chrX: 66763874- | chrX: 66902530- | TGAATAAATAATAGGAAAGAAATAATAGCATCCCAATGATTTTACCTTGGCTCTACCATCATTTGGGGAAGTGATAATTCAGATAGGAGA | 1059 |
| 66944119 | 66902649 | AGTGACTTGGAAGCAGTCTTGAGAGATTGC | |
| chrX: 66763874- | chrX: 66902830- | TGTCATGTGTACCCATTTGTATGTTTTGGTGTGTTTTATGCTTTATGTGATCACCCACATATGCACAGATAATTCCAAAATCCAGTGTGT | 1060 |
| 66944119 | 66902949 | GGGTGTTGTATTCCCTGTGTTAATTATTCA | |
| chrX: 66763874- | chrX: 66903070- | CCTTGAGTTTTGGGAGAGCTTTAGAGAACAAAGACAAGAGACTAAATGATTCTAGATGTAAGAGACAATGTTGCAATAAGTTACTATCCT | 1061 |
| 66944119 | 66903189 | AAAAAGACAGAATACAGGGACAAGAGACTA | |
| chrX: 66763874- | chrX: 66903190- | TTATTTTGGATAGTTTCTTGCTTACCAGTAATACTTAAGTCCTTTACATTAAAAAAAAAAAACTCTGTAAATATATTGCAGAAGAAATCC | 1062 |
| 66944119 | 66903309 | AGACATCCTTCAagattcttagagctggaa | |
| chrX: 66763874- | chrX: 66902590- | ATTTGGGGAAGTGATAATTCAGATAGGAGAAGTGACTTGGAAGCAGTCTTGAGAGATTGCCTGTTCCATCCCCTATCTTTGTCCTTAAAC | 1063 |
| 66944119 | 66902709 | CAAATTGTACAGATAAATAAGGTCTTATTT | |
| chrX: 66763874- | chrX: 66902950- | GTCACACTCAAACACCTATGCACTCACACATACATGAATACACACATGTACATTAGCATGTTTATGCTTATGTTGCATGTGACTGGCAAC | 1064 |
| 66944119 | 66903069 | ATCAGTGCCTTTCTAAGGCAATGTTAACTA | |
| chrX: 66763874- | chrX: 66902650- | CTGTTCCATCCCCTATCTTTGTCCTTAAACCAAATTGTACAGATAAATAAGGTCTTATTTTTAGGACTTACAGAAAAAAGATTCCTTTCA | 1065 |
| 66944119 | 66902769 | TATCCATCTTTGCAATCCTCAACCACTTCT | |
| chrX: 66763874- | chrX: 66902890- | ATGCACAGATAATTCCAAAATCCAGTGTGTGGGTGTTGTATTCCCTGTGTTAATTATTCAGTCACACTCAAACACCTATGCACTCACACA | 1066 |
| 66944119 | 66903009 | TACATGAATACACACATGTACATTAGCATG | |
| chrX: 66763874- | chrX: 66903010- | TTTATGCTTATGTTGCATGTGACTGGCAACATCAGTGCCTTTCTAAGGCAATGTTAACTACCTTGAGTTTTGGGAGAGCTTTAGAGAACA | 1067 |
| 66944119 | 66903129 | AAGACAAGAGACTAAATGATTCTAGATGTA | |
| chrX: 66763874- | chrX: 66902770- | GTCACTATTATGTGTCATTTCAAACATTAAATTCCTCATTCTGCTTTGAAGGAACACATGTGTCATGTGTACCCATTTGTATGTTTTGGT | 1068 |
| 66944119 | 66902889 | GTGTTTTATGCTTTATGTGATCACCCACAT | |
| chrX: 66763874- | chrX: 66903130- | AGAGACAATGTTGCAATAAGTTACTATCCTAAAAAGACAGAATACAGGGACAAGAGACTATTATTTTGGATAGTTTCTTGCTTACCAGTA | 1069 |
| 66944119 | 66903249 | ATACTTAAGTCCTTTACATTAAAAAAAAAA | |
| chrX: 66763874- | chrX: 66903839- | TTGACAAACTAGGGTCCAAGAAAGTATTTTCCTGGGGAAGATGAGATTTCTCAAAGAAGGCACGCACTTTCTAACCTAAGCTTATTTCAG | 1070 |
| 66944119 | 66903958 | TAATCAATGTAACAAGCTGGTCTTGATGAT | |
| chrX: 66763874- | chrX: 66904019- | GAACGCACTAGAATTGGATACAGGACCTGTTTTTGAGGAGCTAACACCCAAAGGCTGAACAGCACTCGTAGCACCGTCCTTTCTGTGCAC | 1071 |
| 66944119 | 66904138 | ATATGGTAGTCCTCAGTTTGCAACAGAAAT | |
| chrX: 66763874- | chrX: 66903899- | CACGCACTTTCTAACCTAAGCTTATTTCAGTAATCAATGTAACAAGCTGGTCTTGATGATTGCAGCAGTACCAATACTGTGGGAGTGTAC | 1072 |
| 66944119 | 66904018 | CAGTTCTAGAACAGCTACAACATTGGAATT | |
| chrX: 66763874- | chrX: 66903599- | ACTTACTCCTGAGTAAATTGTAAAGAATATCTACGTTTTAGGTTGCCTTGTTTTAGACCAAGAGGTACCCAGAGAAAAGGTGTGAACTAT | 1073 |
| 66944119 | 66903718 | GCTAAGGAAATTATCCGAGTTCCAAATTGA | |
| chrX: 66763874- | chrX: 66904079- | AGCACTCGTAGCACCGTCCTTTCTGTGCACATATGGTAGTCCTCAGTTTGCAACAGAAATAAAGCTGTTAGCAAATTATGTGTTCTATTT | 1074 |
| 66944119 | 66904198 | ATGCAAATAAAATCTTGTGGTATGCTAGAA | |
| chrX: 66763874- | chrX: 66903719- | AAAAAAAAAAAAATCATGCTTTTCCGCTATAACCTCTCTCATTCACAGAGTGATTCTCTTTCAGAAGGGCAATCTAGAACTATTATGGGA | 1075 |
| 66944119 | 66903838 | GCCATATTCCATTGGTGGTGCAACCATTTC | |
| chrX: 66763874- | chrX: 66903959- | TGCAGCAGTACCAATACTGTGGGAGTGTACCAGTTCTAGAACAGCTACAACATTGGAATTGAACGCACTAGAATTGGATACAGGACCTGT | 1076 |
| 66944119 | 66904078 | TTTTGAGGAGCTAACACCCAAAGGCTGAAC | |
| chrX: 66763874- | chrX: 66903779- | TCAGAAGGGCAATCTAGAACTATTATGGGAGCCATATTCCATTGGTGGTGCAACCATTTCTTGACAAACTAGGGTCCAAGAAAGTATTTT | 1077 |
| 66944119 | 66903898 | CCTGGGGAAGATGAGATTTCTCAAAGAAGG | |
| chrX: 66763874- | chrX: 66903659- | AGAGGTACCCAGAGAAAAGGTGTGAACTATGCTAAGGAAATTATCCGAGTTCCAAATTGAAAAAAAAAAAAAATCATGCTTTTCCGCTAT | 1078 |
| 66944119 | 66903778 | AACCTCTCTCATTCACAGAGTGATTCTCTT | |
| chrX: 66763874- | chrX: 66904301- | GGGGCCTCCGATTCAGTGTGCTTCTTCAGGTAAGTCACTTCCCTGGAACTCCTCCTTGGAATGAGAGTTGTACTGTTGTGATTTTTAACA | 1079 |
| 66944119 | 66904420 | GTTCCTTCAAGCCAAGCATTTTGGAATCCT | |
| chrX: 66763874- | chrX: 66904682- | TGATGTATAGTTAAATTTATGTTTTTAGTTGTTTTTTTTTCTACTCTCAAATATCAATCACTCTTTAGTTTCTCTTTCTTTTTCCGACCA | 1080 |
| 66944119 | 66904801 | CAAGCATTCTTCCTCTGCTTAAAGAAGCTT | |
| chrX: 66763874- | chrX: 66904802- | CCCTAAAATCCCAGTCTATCCAGTAAGCCAAAGCACAGCAATAAATTTGAGGAAAAAATACCAGGGACTTAGAGACAGAAAGGAGTGAGG | 1081 |
| 66944119 | 66904921 | GGATGCAGAAGCTGAAGCTGGAGCACGGTT | |
| chrX: 66763874- | chrX: 66904442- | gaaagaagaaaggaaAATTAAAGGAAAAAGAACAAATAAAACGTTAAAAAGGAGGAAAGGAAAAAGGATCCTTTACTACAATAAAACTAA | 1082 |
| 66944119 | 66904561 | TCTTATGTTCTTGCAAGTAGCACTTTAAGT | |
| chrX: 66763874- | chrX: 66904562- | AAAAGAAGTTCTTTGCTGACCTGGTTACTACTGAACCTACTACATAAAATAGCCTACTATAATAGATGCATTTATGTGCCTAATCTTCAC | 1083 |
| 66944119 | 66904681 | TTTTTAGGCTTAGTAAAGGGAGAGGAAAGC | |
| chrX: 66763874- | chrX: 66904742- | CTCTTTAGTTTCTCTTTCTTTTTCCGACCACAAGCATTCTTCCTCTGCTTAAAGAAGCTTCCCTAAAATCCCAGTCTATCCAGTAAGCCA | 1084 |
| 66944119 | 66904861 | AAGCACAGCAATAAATTTGAGGAAAAAATA | |
| chrX: 66763874- | chrX: 66904922- | GCAAGCATGAGAAGTTCTGCGTGTTTCAGAGCAGCCAAGGATGTATTTTTGCCTATTCCTGCTGGTGACTCTGTGTGTCTATGCATCCAT | 1085 |
| 66944119 | 66905041 | CTGCTATATTTACATGtttagtcagtcaat | |
| chrX: 66763874- | chrX: 66904502- | AAAAAGGATCCTTTACTACAATAAAACTAATCTTATGTTCTTGCAAGTAGCACTTTAAGTAAAAGAAGTTCTTTGCTGACCTGGTTACTA | 1086 |
| 66944119 | 66904621 | CTGAACCTACTACATAAAATAGCCTACTAT | |
| chrX: 66763874- | chrX: 66904622- | AATAGATGCATTTATGTGCCTAATCTTCACTTTTTAGGCTTAGTAAAGGGAGAGGAAAGCTGATGTATAGTTAAATTTATGTTTTTAGTT | 1087 |
| 66944119 | 66904741 | GTTTTTTTTTCTACTCTCAAATATCAATCA | |
| chrX: 66763874- | chrX: 66904862- | CCAGGGACTTAGAGACAGAAAGGAGTGAGGGGATGCAGAAGCTGAAGCTGGAGCACGGTTGCAAGCATGAGAAGTTCTGCGTGTTTCAGA | 1088 |
| 66944119 | 66904981 | GCAGCCAAGGATGTATTTTTGCCTATTCCT | |
| chrX: 66763874- | chrX: 66905185- | TGACAACTCCAGGTGTTCATGACAGTGATCTTTGTTACTCTGTTGGCTTCATCGAACTTCCTTTTACTTGCTGTGATTCACTACATAGAG | 1089 |
| 66944119 | 66905304 | TGGGCTTTATCTCTGATTTTTATAACCTGC | |
| chrX: 66763874- | chrX: 66905245- | CTTTTACTTGCTGTGATTCACTACATAGAGTGGGCTTTATCTCTGATTTTTATAACCTGCAAGACTGGGGGTATGATCACCAGCAATCTA | 1090 |
| 66944119 | 66905364 | AAAACAGTTAGAAATCCCATGGAGTTATCT | |
| chrX: 66763874- | chrX: 66905365- | TTTGTAGAAATTTTCCTCTACTAATATTATGAAAAATAAGCATCTTATTAGCTCGAGTGTAATTCTATGCATGATTACAGGTATCAATAG | 1091 |
| 66944119 | 66905484 | GAAGAAACATTGACTGAGTTCAAATCTCTT | |
| chrX: 66763874- | chrX: 66905545- | GACTTTTGGTAAAAGTAGAGCACCTTATTTTAAAAACCATTGAGTAGTCCTAATAGTGGAGATATCATCAGGATCTGAATTGTTCATCCC | 1092 |
| 66944119 | 66905664 | TAAAAAAAACACCAATGGAAATCAAACAAT | |
| chrX: 66763874- | chrX: 66905425- | AATTCTATGCATGATTACAGGTATCAATAGGAAGAAACATTGACTGAGTTCAAATCTCTTCTACGCCATGCTAAAGGGGTGACAAGTTCC | 1093 |
| 66944119 | 66905544 | ACAATGGATCATTTTCTCATGGGCATTTCT | |
| chrX: 66763874- | chrX: 66905785- | ATACCCGAAGAAAGAGACTCTGGAAACTCATTATCAGGTCTATCAACTCTTGTATTTGTTCTCCCAGGGAAACAGAAGTACCTGTGCGCC | 1094 |
| 66944119 | 66905904 | AGCAGAAATGATTGCACTATTGATAAATTC | |
| chrX: 66763874- | chrX: 66905485- | CTACGCCATGCTAAAGGGGTGACAAGTTCCACAATGGATCATTTTCTCATGGGCATTTCTGACTTTTGGTAAAAGTAGAGCACCTTATTT | 1095 |
| 66944119 | 66905604 | TAAAAACCATTGAGTAGTCCTAATAGTGGA | |
| chrX: 66763874- | chrX: 66905725- | ATACTCTGTCCACTTTTTTCATGTGGTAGGATATAATTTCATATCTTTTCTGTTCTAGAAATACCCGAAGAAAGAGACTCTGGAAACTCA | 1096 |
| 66944119 | 66905844 | TTATCAGGTCTATCAACTCTTGTATTTGTT | |
| chrX: 66763874- | chrX: 66905305- | AAGACTGGGGGTATGATCACCAGCAATCTAAAAACAGTTAGAAATCCCATGGAGTTATCTTTTGTAGAAATTTTCCTCTACTAATATTAT | 1097 |
| 66944119 | 66905424 | GAAAAATAAGCATCTTATTAGCTCGAGTGT | |
| chrX: 66763874- | chrX: 66905665- | ATAGTGCCAAATTAAACTGTTTGAATATTTAGGTTCTGTATGATCAAATTGTTTGGTGCCATACTCTGTCCACTTTTTTCATGTGGTAGG | 1098 |
| 66944119 | 66905784 | ATATAATTTCATATCTTTTCTGTTCTAGAA | |
| chrX: 66763874- | chrX: 66905845- | CTCCCAGGGAAACAGAAGTACCTGTGCGCCAGCAGAAATGATTGCACTATTGATAAATTCCGAAGGAAAAATTGTCCATCTTGTCGTCTT | 1099 |
| 66944119 | 66905964 | CGGAAATGTTATGAAGCAGGGATGACTCTG | |
| chrX: 66763874- | chrX: 66905125- | TGTAATTCTGAGGATCTCTAGGTCTGAGCATGTGTATGTGTGTGCGCTTCTATGTATCTGTGACAACTCCAGGTGTTCATGACAGTGATC | 1100 |
| 66944119 | 66905244 | TTTGTTACTCTGTTGGCTTCATCGAACTTC | |
| chrX: 66763874- | chrX: 66905605- | GATATCATCAGGATCTGAATTGTTCATCCCTAAAAAAAACACCAATGGAAATCAAACAATATAGTGCCAAATTAAACTGTTTGAATATTT | 1101 |
| 66944119 | 66905724 | AGGTTCTGTATGATCAAATTGTTTGGTGCC | |
| chrX: 66763874- | chrX: 66907790- | GCCTGTGTATAACAGATAGTTTCACTATACTATATAACCGTCAGATGCAGGCTTGTAAATTAATTTGTTGGTGACAATGTTTCAGTACAT | 1102 |
| 66944119 | 66907909 | TTTCAAATTGATTCATTGGTATAGTACTCA | |
| chrX: 66763874- | chrX: 66906650- | CGTTCCTCTATCATAAGGCTCTCTGTCCCACAAACCTGTCTACCATGAGTGTTGTCACCATTCCAGAAAGGCTTGACATCAGTTGATTGA | 1103 |
| 66944119 | 66906769 | GACTTATATTTTCCCTCTCCAAACTCCCCC | |
| chrX: 66763874- | chrX: 66906590- | AAGCATTTTTTTTCAGTAGCACAGTAACGTGATAGATGGAAGATACAGCTCTTTCAAGGGCGTTCCTCTATCATAAGGCTCTCTGTCCCA | 1104 |
| 66944119 | 66906709 | CAAACCTGTCTACCATGAGTGTTGTCACCA | |
| chrX: 66763874- | chrX: 66906470- | TCTCTTTGGAGCCTGTGACTAGGCTGCCACACAGAGCCAATTTCCTATCCTATCTCTCCCAAAGATGAGCAGGTGTTTTAATAATTTCCT | 1105 |
| 66944119 | 66906589 | TTTCTTTGCAAAGCTATTGACCATTTCCAA | |
| chrX: 66763874- | chrX: 66907310- | AAGCATCCACATCAAATGAGACTCAGATATCTGAGAAAACTCAACCTTGTTTTGGTTTGCTTGGTGCACCCCAAAGAAATCCAACAATTG | 1106 |
| 66944119 | 66907429 | AGGTCTACAGTGGAGAAGAAGTAGGACTGG | |
| chrX: 66763874- | chrX: 66907010- | TCTAATTTAGACCAGTTGTCTATCTCTGGCTTTCTGTGAGGTGTTCAATACCTTGTCTGCCTATGTGCACATTTATAGACAACAACTAGT | 1107 |
| 66944119 | 66907129 | TCTCTTATCCTGGAGCAGGGCCATGTGTGG | |
| chrX: 66763874- | chrX: 66906290- | TGTTGTTCAGTGGTAGCTAGGATGAGAGAACTAAGAAATCCAGAACAGTCAGAGGTGCAGGATGACCCAGGCATAGGCGCAGGATGACCC | 1108 |
| 66944119 | 66906409 | AGGCACAGGCTGATCCTGAACACCTGGGAA | |
| chrX: 66763874- | chrX: 66907490- | TTCTCCATATGAATGTTGCAGCCCCATGTTGAGGGTTCTTATACACTCAACTGTCAATTATTTAGCCTTCTGTGAATTATGTATAGTATA | 1109 |
| 66944119 | 66907609 | AAAGATAGGGACTCTCAAGTAGGGAACCTC | |
| chrX: 66763874- | chrX: 66906050- | TTCTTCTTTGATGCTTCCAAGGGGACCAGCCATGCTCTAGACACAGGCTGACCCTTTCATAGGCAACGTGGCCATCAGCCAGCTGGTGCC | 1110 |
| 66944119 | 66906169 | TTTTTTTTAATCCTTATCTATACCAATCCC | |
| chrX: 66763874- | chrX: 66906770- | ATCTCTTCATGTTTACATCTGCCCAATGCCAGGGTCCTCGCTGCTGCCTGCTACTTCCAAAAAGATGTGTCTTTCATGAGAAAAACAAGA | 1111 |
| 66944119 | 66906889 | TCATTAATCCACTTCGATTTGGAAATGGAA | |
| chrX: 66763874- | chrX: 66906830- | AAAGATGTGTCTTTCATGAGAAAAACAAGATCATTAATCCACTTCGATTTGGAAATGGAATTTGAAGAAAGGCAAGCCTATTTCTGAGTG | 1112 |
| 66944119 | 66906949 | CCTGCAACTGTAGCCTCATACCCAATTATT | |
| chrX: 66763874- | chrX: 66906410- | TATCCCTTAGCTAACTGCTGCCTATGTTGTAGGGCCAGCCACCTCGAATGAGAAGCTACTTCTCTTTGGAGCCTGTGACTAGGCTGCCAC | 1113 |
| 66944119 | 66906529 | ACAGAGCCAATTTCCTATCCTATCTCTCCC | |
| chrX: 66763874- | chrX: 66906890- | TTTGAAGAAAGGCAAGCCTATTTCTGAGTGCCTGCAACTGTAGCCTCATACCCAATTATTCATTATTAGCCTGGAAAACCCAAGTGCCTA | 1114 |
| 66944119 | 66907009 | GAATCCAACCCTCTCCCCTCTCCTCTTAAG | |
| chrX: 66763874- | chrX: 66907430- | GGTCAGGGAGTACAGAGGCAAAGGCAGGAAGGGTGACAAAGTGATTGACAAGAAAAAATGTTCTCCATATGAATGTTGCAGCCCCATGTT | 1115 |
| 66944119 | 66907549 | GAGGGTTCTTATACACTCAACTGTCAATTA | |
| chrX: 66763874- | chrX: 66907130- | ATCTTCATATAGATAACTATATCCTCCCCATCCTCACAGGGCAGTAGTATTATTTAAACAGAACAAAGTACCTCACATGAATTGACCCAG | 1116 |
| 66944119 | 66907249 | GCTGGATGAGAGACAATTTCAAAAGAATCA | |
| chrX: 66763874- | chrX: 66907850- | TAATTTGTTGGTGACAATGTTTCAGTACATTTTCAAATTGATTCATTGGTATAGTACTCAAATTTGAGTGGGCTTGGTGAACACAATGAA | 1117 |
| 66944119 | 66907969 | GACAAGCTGAGAAGTGCTGTGACTGGCCTT | |
| chrX: 66763874- | chrX: 66906350- | GATGACCCAGGCATAGGCGCAGGATGACCCAGGCACAGGCTGATCCTGAACACCTGGGAATATCCCTTAGCTAACTGCTGCCTATGTTGT | 1118 |
| 66944119 | 66906469 | AGGGCCAGCCACCTCGAATGAGAAGCTACT | |
| chrX: 66763874- | chrX: 66907610- | TTGGCTTGCCATCTGGCAATATGAATTGCAAGTCCACTTTGATGCAGGTAAAGTTTAATGGTAACAAAAGTCCTCATAACATTTGGATGC | 1119 |
| 66944119 | 66907729 | AAATCTTAACATTAATTCCATGTCTCAGCC | |
| chrX: 66763874- | chrX: 66907670- | GTAACAAAAGTCCTCATAACATTTGGATGCAAATCTTAACATTAATTCCATGTCTCAGCCAACATTCTCCATTATTAAGCAGCCTGTGAT | 1120 |
| 66944119 | 66907789 | GTGATTACAGTGAACCACTTTTGAAAAGGA | |
| chrX: 66763874- | chrX: 66907190- | GAACAAAGTACCTCACATGAATTGACCCAGGCTGGATGAGAGACAATTTCAAAAGAATCATCTCAAGTAGCGTCCAGTACTCCCAAACAT | 1121 |
| 66944119 | 66907309 | CACAGGTAGATGTTCTGTGAGTGGCTTTCC | |
| chrX: 66763874- | chrX: 66906230- | GTGAGTGAGGATGCATCTGACTGGGCAGGGCCCCCAGGGGACTTAATGATACTGGCCTGATGTTGTTCAGTGGTAGCTAGGATGAGAGAA | 1122 |
| 66944119 | 66906349 | CTAAGAAATCCAGAACAGTCAGAGGTGCAG | |
| chrX: 66763874- | chrX: 66906170- | CATTCCGGGGCTCAGCATTAGAGCAGGCGGTGTGAAGCAGGGATCAGGAGCCAACAGAAGGTGAGTGAGGATGCATCTGACTGGGCAGGG | 1123 |
| 66944119 | 66906289 | CCCCCAGGGGACTTAATGATACTGGCCTGA | |
| chrX: 66763874- | chrX: 66907370- | TTGGTGCACCCCAAAGAAATCCAACAATTGAGGTCTACAGTGGAGAAGAAGTAGGACTGGGGTCAGGGAGTACAGAGGCAAAGGCAGGAA | 1124 |
| 66944119 | 66907489 | GGGTGACAAAGTGATTGACAAGAAAAAATG | |
| chrX: 66763874- | chrX: 66907550- | TTTAGCCTTCTGTGAATTATGTATAGTATAAAAGATAGGGACTCTCAAGTAGGGAACCTCTTGGCTTGCCATCTGGCAATATGAATTGCA | 1125 |
| 66944119 | 66907669 | AGTCCACTTTGATGCAGGTAAAGTTTAATG | |
| chrX: 66763874- | chrX: 66906530- | AAAGATGAGCAGGTGTTTTAATAATTTCCTTTTCTTTGCAAAGCTATTGACCATTTCCAAAAGCATTTTTTTTCAGTAGCACAGTAACGT | 1126 |
| 66944119 | 66906649 | GATAGATGGAAGATACAGCTCTTTCAAGGG | |
| chrX: 66763874- | chrX: 66906110- | AGGCAACGTGGCCATCAGCCAGCTGGTGCCTTTTTTTTAATCCTTATCTATACCAATCCCCATTCCGGGGCTCAGCATTAGAGCAGGCGG | 1127 |
| 66944119 | 66906229 | TGTGAAGCAGGGATCAGGAGCCAACAGAAG | |
| chrX: 66763874- | chrX: 66906710- | TTCCAGAAAGGCTTGACATCAGTTGATTGAGACTTATATTTTCCCTCTCCAAACTCCCCCATCTCTTCATGTTTACATCTGCCCAATGCC | 1128 |
| 66944119 | 66906829 | AGGGTCCTCGCTGCTGCCTGCTACTTCCAA | |
| chrX: 66763874- | chrX: 66907730- | AACATTCTCCATTATTAAGCAGCCTGTGATGTGATTACAGTGAACCACTTTTGAAAAGGAGCCTGTGTATAACAGATAGTTTCACTATAC | 1129 |
| 66944119 | 66907849 | TATATAACCGTCAGATGCAGGCTTGTAAAT | |
| chrX: 66763874- | chrX: 66907070- | CTATGTGCACATTTATAGACAACAACTAGTTCTCTTATCCTGGAGCAGGGCCATGTGTGGATCTTCATATAGATAACTATATCCTCCCCA | 1130 |
| 66944119 | 66907189 | TCCTCACAGGGCAGTAGTATTATTTAAACA | |
| chrX: 66763874- | chrX: 66906950- | CATTATTAGCCTGGAAAACCCAAGTGCCTAGAATCCAACCCTCTCCCCTCTCCTCTTAAGTCTAATTTAGACCAGTTGTCTATCTCTGGC | 1131 |
| 66944119 | 66907069 | TTTCTGTGAGGTGTTCAATACCTTGTCTGC | |
| chrX: 66763874- | chrX: 66907250- | TCTCAAGTAGCGTCCAGTACTCCCAAACATCACAGGTAGATGTTCTGTGAGTGGCTTTCCAAGCATCCACATCAAATGAGACTCAGATAT | 1132 |
| 66944119 | 66907369 | CTGAGAAAACTCAACCTTGTTTTGGTTTGC | |
| chrX: 66763874- | chrX: 66908351- | TCCCGATGGCTTTTACCCTAAGTAACTTGGTATGCCATATAATATGTAACAGCACCAACAGGCAGAGAATCGCCAGAAAACACTCTTGAT | 1133 |
| 66944119 | 66908470 | TACCTCAAACGAAAAAGTACCACCAGGATC | |
| chrX: 66763874- | chrX: 66908591- | TGCTGAATGAACTTATCTCCACGTTCCCTGCCCTACTGACACAACCCCCTCCCAAGTTTATTGTTAACTTACACATTCAATGCACAGCAC | 1134 |
| 66944119 | 66908710 | ACCTTTACTCAAACAATGGAAAAGAAAGAA | |
| chrX: 66763874- | chrX: 66908531- | AGTAAAAACCCAATTGTTTACCTTCTTAAATCACTGCTTGAAGAGCAAATCTTTCCATTTTGCTGAATGAACTTATCTCCACGTTCCCTG | 1135 |
| 66944119 | 66908650 | CCCTACTGACACAACCCCCTCCCAAGTTTA | |
| chrX: 66763874- | chrX: 66908291- | gggcttaatatGACATTGAGGTCACTAGTAATTTAGCTGGAAAGTCTGTAACACAGCACTTCCCGATGGCTTTTACCCTAAGTAACTTGG | 1136 |
| 66944119 | 66908410 | TATGCCATATAATATGTAACAGCACCAACA | |
| chrX: 66763874- | chrX: 66908471- | CTGTTCAGAAGCTAATTTTAGTAATTAAGGGAATCATATGCTATGTTCAAATACCATGCCAGTAAAAACCCAATTGTTTACCTTCTTAAA | 1137 |
| 66944119 | 66908590 | TCACTGCTTGAAGAGCAAATCTTTCCATTT | |
| chrX: 66763874- | chrX: 66908411- | GGCAGAGAATCGCCAGAAAACACTCTTGATTACCTCAAACGAAAAAGTACCACCAGGATCCTGTTCAGAAGCTAATTTTAGTAATTAAGG | 1138 |
| 66944119 | 66908530 | GAATCATATGCTATGTTCAAATACCATGCC | |
| chrX: 66763874- | chrX: 66908711- | AGTGTCAATTCAAAGTGGCCCTTGTCTATTCCTTAAGGAGTAGACTTCCATTTTCATCAGATTTGGATTTAGCATAGACATATTGATTAC | 1139 |
| 66944119 | 66908830 | CTTGAAGAAGAATTCATATAATTTTATCTT | |
| chrX: 66763874- | chrX: 66908651- | TTGTTAACTTACACATTCAATGCACAGCACACCTTTACTCAAACAATGGAAAAGAAAGAAAGTGTCAATTCAAAGTGGCCCTTGTCTATT | 1140 |
| 66944119 | 66908770 | CCTTAAGGAGTAGACTTCCATTTTCATCAG | |
| chrX: 66763874- | chrX: 66908771- | ATTTGGATTTAGCATAGACATATTGATTACCTTGAAGAAGAATTCATATAATTTTATCTTCTGATTCCCATCACTCAAATCAAAATTACA | 1141 |
| 66944119 | 66908890 | TAATATATTCCAAAATGGCaactaggaatg | |
| chrX: 66763874- | chrX: 66909507- | TTTCTTTGTAGATGATTCATTCCTGGCTGCATTTGAAAACCACATATTGTTAATTGCTTGACGAATTTAAATCCCTTGACTACTTTTCAT | 1142 |
| 66944119 | 66909626 | TTCAGAAAACACTTACAAAAAAAGTCCAAA | |
| chrX: 66763874- | chrX: 66909687- | TGTAAAGCCATTTCTCATTTTTCTCCGCACTTTCCAAGGGTACACTCCTTGTTTCCAAGATGGAATGAGAAATAAAGAAGTGCCCTTCCT | 1143 |
| 66944119 | 66909806 | GCCATCTTCTCCCCTGACCCTTTCCTCCTT | |
| chrX: 66763874- | chrX: 66909747- | TGGAATGAGAAATAAAGAAGTGCCCTTCCTGCCATCTTCTCCCCTGACCCTTTCCTCCTTCCCACTTTCCTCCTATTCCTCCCCAAACAT | 1144 |
| 66944119 | 66909866 | GATTTATTTCTGCGTTTTGCAACTCTTGAG | |
| chrX: 66763874- | chrX: 66909087- | AGTGCTTTGATACATCGTAATTGTATCTACCTCCATTCACACCTACTTAAGATATCTGTCTAAAAGTAGACTAGACAGATTATTCAGAGA | 1145 |
| 66944119 | 66909206 | GTGGAGGGCAGAAGGGCTGTCTCTGTATCT | |
| chrX: 66763874- | chrX: 66909147- | TAAAAGTAGACTAGACAGATTATTCAGAGAGTGGAGGGCAGAAGGGCTGTCTCTGTATCTTAAAGAAGCTGGCACTCTTCAGCTGATGGC | 1146 |
| 66944119 | 66909266 | TGCTTGGTCTTGAGGCCTCAAGATCTTTAA | |
| chrX: 66763874- | chrX: 66909627- | TGAGGACCTTCCCTCCAGTGAATTAGCTGTGGCTTTCTCACAGTCCATAGTTAGGATAAATGTAAAGCCATTTCTCATTTTTCTCCGCAC | 1147 |
| 66944119 | 66909746 | TTTCCAAGGGTACACTCCTTGTTTCCAAGA | |
| chrX: 66763874- | chrX: 66909927- | AAGGTAGTAGGCCTTTCAGAAATTTCAGGTAGCAGCCAAACCCCAGAAGAAGAGAAGGAACACAGAGACCTAGACCATGTGAGAACCTGA | 1148 |
| 66944119 | 66910046 | GGTGTGCAGCATTTACTTCACAGATTCGTC | |
| chrX: 66763874- | chrX: 66909567- | ACGAATTTAAATCCCTTGACTACTTTTCATTTCAGAAAACACTTACAAAAAAAGTCCAAATGAGGACCTTCCCTCCAGTGAATTAGCTGT | 1149 |
| 66944119 | 66909686 | GGCTTTCTCACAGTCCATAGTTAGGATAAA | |
| chrX: 66763874- | chrX: 66909987- | CACAGAGACCTAGACCATGTGAGAACCTGAGGTGTGCAGCATTTACTTCACAGATTCGTCTAGCATATTTGAGAGGTGTCTTTCCTACTA | 1150 |
| 66944119 | 66910106 | GGAGACTGAACTCTGCATCTGAGAATAAAA | |
| chrX: 66763874- | chrX: 66909867- | TTCTCAGCATTTAGTAAATGGTGTTGGTCCCTGTTGATTCCTTCCTCTCCTGGACCATGGAAGGTAGTAGGCCTTTCAGAAATTTCAGGT | 1151 |
| 66944119 | 66909986 | AGCAGCCAAACCCCAGAAGAAGAGAAGGAA | |
| chrX: 66763874- | chrX: 66910047- | TAGCATATTTGAGAGGTGTCTTTCCTACTAGGAGACTGAACTCTGCATCTGAGAATAAAAACTTAACATATCTACAGGTTTTGACAACCT | 1152 |
| 66944119 | 66910166 | CTGTGAATTATCTAGTTGAGAGGATGGCTC | |
| chrX: 66763874- | chrX: 66909387- | TTTTTTTCTAGCAGCTGTTGTTGTTTCTGAAAGAATCTTGAGGGTGTTTGGAGTCTCAGAATGGCTTCCTTAAAGACTACCTTCAGACTC | 1153 |
| 66944119 | 66909506 | TCAGCTGCTCATCCACAACAGAGATCAGCC | |
| chrX: 66763874- | chrX: 66909267- | TCTGGCTTTCTCTATAGTGTTTCATTCACTGTTTGGTGATGGAATCTCTTCAGTTCAGAGATACTTAATAGATATAGCTTTTTCTTTCCT | 1154 |
| 66944119 | 66909386 | GCTTCCAGGCCTACCTACCTGTTTCTTGCT | |
| chrX: 66763874- | chrX: 66909327- | ATACTTAATAGATATAGCTTTTTCTTTCCTGCTTCCAGGCCTACCTACCTGTTTCTTGCTTTTTTTTCTAGCAGCTGTTGTTGTTTCTGA | 1155 |
| 66944119 | 66909446 | AAGAATCTTGAGGGTGTTTGGAGTCTCAGA | |
| chrX: 66763874- | chrX: 66909447- | ATGGCTTCCTTAAAGACTACCTTCAGACTCTCAGCTGCTCATCCACAACAGAGATCAGCCTTTCTTTGTAGATGATTCATTCCTGGCTGC | 1156 |
| 66944119 | 66909566 | ATTTGAAAACCACATATTGTTAATTGCTTG | |
| chrX: 66763874- | chrX: 66910107- | ACTTAACATATCTACAGGTTTTGACAACCTCTGTGAATTATCTAGTTGAGAGGATGGCTCAAGGAGCCTATTGCCATGGTCTGATGTCGT | 1157 |
| 66944119 | 66910226 | TATGGACGCTATGAACATCCTTGCAGTTTC | |
| chrX: 66763874- | chrX: 66909027- | TTTCAGCCCTTTCTTAGATTGTCTGTTCCCTGCTCCCAGAAGTATAGATAGTGAGACTTGAGTGCTTTGATACATCGTAATTGTATCTAC | 1158 |
| 66944119 | 66909146 | CTCCATTCACACCTACTTAAGATATCTGTC | |
| chrX: 66763874- | chrX: 66909207- | TAAAGAAGCTGGCACTCTTCAGCTGATGGCTGCTTGGTCTTGAGGCCTCAAGATCTTTAATCTGGCTTTCTCTATAGTGTTTCATTCACT | 1159 |
| 66944119 | 66909326 | GTTTGGTGATGGAATCTCTTCAGTTCAGAG | |
| chrX: 66763874- | chrX: 66909807- | CCCACTTTCCTCCTATTCCTCCCCAAACATGATTTATTTCTGCGTTTTGCAACTCTTGAGTTCTCAGCATTTAGTAAATGGTGTTGGTCC | 1160 |
| 66944119 | 66909926 | CTGTTGATTCCTTCCTCTCCTGGACCATGG | |
| chrX: 66763874- | chrX: 66910167- | AAGGAGCCTATTGCCATGGTCTGATGTCGTTATGGACGCTATGAACATCCTTGCAGTTTCCATTGTTGAAGACAGCCCTGATGCCAGCTG | 1161 |
| 66944119 | 66910286 | TCTCATCATTCCCCATGTTCAAGAGCATCC | |
| chrX: 66763874- | chrX: 66910227- | CATTGTTGAAGACAGCCCTGATGCCAGCTGTCTCATCATTCCCCATGTTCAAGAGCATCCCAGCATTGCTACCTCAGGATCCCATGTCCT | 1162 |
| 66944119 | 66910346 | GAATGCAACAGAGTGATTTCGCTGCTGAAT | |
| chrX: 66763874- | chrX: 66910919- | GGCAGCAGCACAAATGTTTTGTTGATGAGGGTTTAAATTGTAGAAAGTGAGACAATTTTAGGAAGGCCAGCTAGAGAGAAATTTCTAGCA | 1163 |
| 66944119 | 66911038 | TCAAATTTTGCTAAACACCTAGGATTTGTA | |
| chrX: 66763874- | chrX: 66910799- | CATTGAAAGCAAACTTGGTTATTATACTAGGTAGTTTAGACTTCAAGCAGTTGAAAATCTTTGAGCATGGGATAGGCATGATGACATTGT | 1164 |
| 66944119 | 66910918 | GTTTATTTGCATGTTTCTTTAAAGAAAACT | |
| chrX: 66763874- | chrX: 66911039- | GTTACCTCCATTTGGGTTGTTACCTGCAAGTACTGACCACGTATATGAAGAAGTACTGGTTTAGACCAAGGCAATTGGCTTGTATAAGAG | 1165 |
| 66944119 | 66911158 | GCCTACCCTCATACCAAAAGCCAGTTTCCT | |
| chrX: 66763874- | chrX: 66910859- | TTGAGCATGGGATAGGCATGATGACATTGTGTTTATTTGCATGTTTCTTTAAAGAAAACTGGCAGCAGCACAAATGTTTTGTTGATGAGG | 1166 |
| 66944119 | 66910978 | GTTTAAATTGTAGAAAGTGAGACAATTTTA | |
| chrX: 66763874- | chrX: 66910979- | GGAAGGCCAGCTAGAGAGAAATTTCTAGCATCAAATTTTGCTAAACACCTAGGATTTGTAGTTACCTCCATTTGGGTTGTTACCTGCAAG | 1167 |
| 66944119 | 66911098 | TACTGACCACGTATATGAAGAAGTACTGGT | |
| chrX: 66763874- | chrX: 66912145- | AGTGGGAAGTCTCATTTCTGAGAAGTGCTGCTTCCTACCCACAATTCTTTGATAGCTGAGTGCTTTAGCTGATCTGCATAACTGAGGTGT | 1168 |
| 66944119 | 66912264 | GCACCAAGGAGCAGAATTACTCTATAAATT | |
| chrX: 66763874- | chrX: 66911725- | TGCCCAAGTTATCTAATGTCTGCAGGTACATATTCCTGGCCTAAGGATTGTGCTAAAGAAGTTATTTCTAAGAAATATAGTGACTTCCAG | 1169 |
| 66944119 | 66911844 | CATCATGCAGAATGACCATTTAATATTTTG | |
| chrX: 66763874- | chrX: 66912205- | TGCTTTAGCTGATCTGCATAACTGAGGTGTGCACCAAGGAGCAGAATTACTCTATAAATTTTGGCATCAACATGTGCAACTTGTGACTCA | 1170 |
| 66944119 | 66912324 | GCACTTTGAAACTCTGGGGATTTTTTTGTT | |
| chrX: 66763874- | chrX: 66911545- | TAAGAATCAGTCAATAATGCATTAATGATCAAAAGCAGACCATCCTTACCACATGGTGCATAAGATTATGCTATTATGCTATTAGCTACT | 1171 |
| 66944119 | 66911664 | AATGCCACTAAAGTTAATTATGTTGGGTCT | |
| chrX: 66763874- | chrX: 66911605- | TAAGATTATGCTATTATGCTATTAGCTACTAATGCCACTAAAGTTAATTATGTTGGGTCTGCAACGTTGTCATACACAAAGGATAGGATG | 1172 |
| 66944119 | 66911724 | CAAAACTGTCCTAGGCCAAAGCATGGTTAT | |
| chrX: 66763874- | chrX: 66911665- | GCAACGTTGTCATACACAAAGGATAGGATGCAAAACTGTCCTAGGCCAAAGCATGGTTATTGCCCAAGTTATCTAATGTCTGCAGGTACA | 1173 |
| 66944119 | 66911784 | TATTCCTGGCCTAAGGATTGTGCTAAAGAA | |
| chrX: 66763874- | chrX: 66911845- | AATATCTAGACATTCTGCTGTAGAATTTAATAGTCCTTTTATACACTGTCTGACCAACATTTTGACATTTACTCAGAACCCCATCACAGT | 1174 |
| 66944119 | 66911964 | GCTACCACATAACCTCATTGCTAAAGTGGG | |
| chrX: 66763874- | chrX: 66912085- | CTTCCATTCTCCTTTGATTCTCTAGACTAGAATTCCAAAGACCCTCAGGCTGGTGATGCAAGTGGGAAGTCTCATTTCTGAGAAGTGCTG | 1175 |
| 66944119 | 66912204 | CTTCCTACCCACAATTCTTTGATAGCTGAG | |
| chrX: 66763874- | chrX: 66911485- | ATAGGCAGAGAATACCTATACCTCTAGCTCAGGTCATCATTTTGCAGATGTGTGTGTCATTAAGAATCAGTCAATAATGCATTAATGATC | 1176 |
| 66944119 | 66911604 | AAAAGCAGACCATCCTTACCACATGGTGCA | |
| chrX: 66763874- | chrX: 66912025- | TTCAGCAGGCAGCAGAGTGTCATAAAGAATTAACAACGTGGAACTCAGTTACTGGGATTTCTTCCATTCTCCTTTGATTCTCTAGACTAG | 1177 |
| 66944119 | 66912144 | AATTCCAAAGACCCTCAGGCTGGTGATGCA | |
| chrX: 66763874- | chrX: 66911965- | AGGCCTAGAAATCACAGATTTGTAGAAACCATCCAATGATTGAATCCCCTCTACTTCCTGTTCAGCAGGCAGCAGAGTGTCATAAAGAAT | 1178 |
| 66944119 | 66912084 | TAACAACGTGGAACTCAGTTACTGGGATTT | |
| chrX: 66763874- | chrX: 66911905- | TTTGACATTTACTCAGAACCCCATCACAGTGCTACCACATAACCTCATTGCTAAAGTGGGAGGCCTAGAAATCACAGATTTGTAGAAACC | 1179 |
| 66944119 | 66912024 | ATCCAATGATTGAATCCCCTCTACTTCCTG | |
| chrX: 66763874- | chrX: 66911785- | GTTATTTCTAAGAAATATAGTGACTTCCAGCATCATGCAGAATGACCATTTAATATTTTGAATATCTAGACATTCTGCTGTAGAATTTAA | 1180 |
| 66944119 | 66911904 | TAGTCCTTTTATACACTGTCTGACCAACAT | |
| chrX: 66763874- | chrX: 66912693- | TTGGTCTGTAAATTACCCTTGAAACAACCCTTGAAATTTCCACTCCATGACCTAAATCGTCATCCCTAAATTGGTTACATACATATTTGG | 1181 |
| 66944119 | 66912812 | TGACACTTTGGAGGGGAAAAGCTTTATGTC | |
| chrX: 66763874- | chrX: 66912993- | TACCGCATATCTGATACTTGAATGAGTACCTCCTTGTAAAATTTATACTTAAATCCTTGAGTTTTTAAAGTGTAATAGCAATAGAAAGAT | 1182 |
| 66944119 | 66913112 | TTTATTGTTGTTTACTTTTACTGTGAGTGC | |
| chrX: 66763874- | chrX: 66912873- | ATTCCTCCAAACCAAATTATCTGGGATAGCACATATATGTTGTACTCTGTCTCTGAGCATTTGCTCTTAGAGAACTATGGTTAGAGCGAA | 1183 |
| 66944119 | 66912992 | GTAAATTTTTCTAATCATAAAAATTAATGA | |
| chrX: 66763874- | chrX: 66912753- | CATCCCTAAATTGGTTACATACATATTTGGTGACACTTTGGAGGGGAAAAGCTTTATGTCTCTCTAACTGTAGTTCTTAAGGGAATTTGC | 1184 |
| 66944119 | 66912872 | ATATGGAAAAAACAGAGACTGCGTCTCTTA | |
| chrX: 66763874- | chrX: 66912933- | TTGCTCTTAGAGAACTATGGTTAGAGCGAAGTAAATTTTTCTAATCATAAAAATTAATGATACCGCATATCTGATACTTGAATGAGTACC | 1185 |
| 66944119 | 66913052 | TCCTTGTAAAATTTATACTTAAATCCTTGA | |
| chrX: 66763874- | chrX: 66913173- | AGGTAGTAGGCAGAAAACTGAGTACACAGCACACAATAGGCCATATATACAAAAGCAAGTATTTTGCAAATAATAATAATTCAGGAAAAA | 1186 |
| 66944119 | 66913292 | AGCTTCACTTTCGTTGGTAACCTGTTTGTT | |
| chrX: 66763874- | chrX: 66912633- | GGTCTGTTCTCTCTCTCTCTGCTGTTACAGTGTGGCTTTTGGTTCTTGTTTCTTTGTTCTTTGGTCTGTAAATTACCCTTGAAACAACCC | 1187 |
| 66944119 | 66912752 | TTGAAATTTCCACTCCATGACCTAAATCGT | |
| chrX: 66763874- | chrX: 66913113- | TCCAAAATCCCTCAGTTGCTCTTGAAAGAGCAAGATGATGCCATAGGCAATATTTTCCAAAGGTAGTAGGCAGAAAACTGAGTACACAGC | 1188 |
| 66944119 | 66913232 | ACACAATAGGCCATATATACAAAAGCAAGT | |
| chrX: 66763874- | chrX: 66912813- | TCTCTAACTGTAGTTCTTAAGGGAATTTGCATATGGAAAAAACAGAGACTGCGTCTCTTAATTCCTCCAAACCAAATTATCTGGGATAGC | 1189 |
| 66944119 | 66912932 | ACATATATGTTGTACTCTGTCTCTGAGCAT | |
| chrX: 66763874- | chrX: 66913053- | GTTTTTAAAGTGTAATAGCAATAGAAAGATTTTATTGTTGTTTACTTTTACTGTGAGTGCTCCAAAATCCCTCAGTTGCTCTTGAAAGAG | 1190 |
| 66944119 | 66913172 | CAAGATGATGCCATAGGCAATATTTTCCAA | |
| chrX: 66763874- | chrX: 66912573- | TACCCTTCAGCCCTTCTTTTGTGGATGAAAAATGGTCTTTGTGCTCCCAGTCACCACTGGGGTCTGTTCTCTCTCTCTCTGCTGTTACAG | 1191 |
| 66944119 | 66912692 | TGTGGCTTTTGGTTCTTGTTTCTTTGTTCT | |
| chrX: 66763874- | chrX: 66914335- | TCTTACAAGTGGATAGGAAAAGAAAAACCCCCAGTAAAAAATGGCAACCGCCCACCTCCCCAACTTTACATGCTGCTTCCTATGTTAGAG | 1192 |
| 66944119 | 66914454 | GATCTGTCTTAGGCATCTGATTATGGAGCC | |
| chrX: 66763874- | chrX: 66913495- | AACACGCAGCCACCCAGAAATCGGTAGAGCCTTCAGCTCATCTGTTATTAATATTTCTGTGACAACAGATATCTAGGAAGTAAACAGGAA | 1193 |
| 66944119 | 66913614 | ATTGCATCGCTATCCTGCATCACCTTTTTT | |
| chrX: 66763874- | chrX: 66914155- | CTCTTCTCCTACATCTTCTCACTCCCCACCCCACCCCCACATACACACAATTCTTGTCCACTATGTTCAGAGAGATGCACGCACACATAT | 1194 |
| 66944119 | 66914274 | ATATGTATATATATAGTATATTTGTCAATA | |
| chrX: 66763874- | chrX: 66913555- | GACAACAGATATCTAGGAAGTAAACAGGAAATTGCATCGCTATCCTGCATCACCTTTTTTGGAATCAGGTTCCATTCTTCTCAGTCCAGT | 1195 |
| 66944119 | 66913674 | TCAACCTTGTGATACTTTTTAGATCTCAAC | |
| chrX: 66763874- | chrX: 66913975- | GCTTTGATTAGTGAGCTGTTAGGCACACAGACATTAATTTCAAAGCATTCTCATCTCCAGTCTGAGTAATAATGCTTATAGTATTATGCA | 1196 |
| 66944119 | 66914094 | ATTGTTTGGCTGCTGCAAGAAATTCAGCAG | |
| chrX: 66763874- | chrX: 66913615- | GGAATCAGGTTCCATTCTTCTCAGTCCAGTTCAACCTTGTGATACTTTTTAGATCTCAACCAAGGCATAGAAATATATTTTCCCTTGCTT | 1197 |
| 66944119 | 66913734 | AATACCCCATGGAACCAATGCCCCTGTGGT | |
| chrX: 66763874- | chrX: 66913915- | GCCACAATGAAACTGAAAGAGACTGATGACTCTCCTCAGGGTGGAAAATGAGGCATGGAAGCTTTGATTAGTGAGCTGTTAGGCACACAG | 1198 |
| 66944119 | 66914034 | ACATTAATTTCAAAGCATTCTCATCTCCAG | |
| chrX: 66763874- | chrX: 66913795- | ATGTAAGCACCGAGCACCTGCAGAAAACTTGGACTGGCATTTGGATCTAAGAAGAAAATCTGCATCTTGACCAAGATGAAAAGTCACCAG | 1199 |
| 66944119 | 66913914 | CCCAAGCTTGTGCAGTGAAGTGTCATGTTG | |
| chrX: 66763874- | chrX: 66913435- | TTTTGGAGACATTTGCACATCTTTTGGGATCACGTTGTTAAGAAGTAGAACTAAGGGAAAAACACGCAGCCACCCAGAAATCGGTAGAGC | 1200 |
| 66944119 | 66913554 | CTTCAGCTCATCTGTTATTAATATTTCTGT | |
| chrX: 66763874- | chrX: 66914635- | GCGTCTGAGGCTTAGGAGCTTAGGTTTTTGCTCCTCAACACAGACTTTGACGTTGGGGTTGGGGGCTACTCTCTTGATTGCTGACTCCCT | 1201 |
| 66944119 | 66914754 | CCAGCGGGACCAATAGTGTTTTcctacctc | |
| chrX: 66763874- | chrX: 66914395- | CAACTTTACATGCTGCTTCCTATGTTAGAGGATCTGTCTTAGGCATCTGATTATGGAGCCTGCTAGATACAAGCCCGTATTTAGACTGCT | 1202 |
| 66944119 | 66914514 | ACAGTCAACAATGTCTCTCTTTCATACTAG | |
| chrX: 66763874- | chrX: 66914515- | AAAAATTCCGGGTTGGCAATTGCAAGCATCTCAAAATGACCAGACCCTGAAGAAAGGCTGACTTGCCTCATTCAAAATGAGGGCTCTAGA | 1203 |
| 66944119 | 66914634 | GGGCTCTAGTGGATAGTCTGGAGAAACCTG | |
| chrX: 66763874- | chrX: 66913315- | atttaaaaaGAGTGTCACTTGTTACAGATTGTGGGATGTGTTCCTTAAGATCACAAAAATGTAAAATATTTTCTTTTTATACTGAACACA | 1204 |
| 66944119 | 66913434 | TGCATAGACAACTTACCTGAGCAAGCTGCT | |
| chrX: 66763874- | chrX: 66913735- | TGAAGTAAAAATTGATTGTTGAGGGACATTTCAGCCCTCTAGCAGTCAACAATTAAAAACATGTAAGCACCGAGCACCTGCAGAAAACTT | 1205 |
| 66944119 | 66913854 | GGACTGGCATTTGGATCTAAGAAGAAAATC | |
| chrX: 66763874- | chrX: 66914455- | TGCTAGATACAAGCCCGTATTTAGACTGCTACAGTCAACAATGTCTCTCTTTCATACTAGAAAAATTCCGGGTTGGCAATTGCAAGCATC | 1206 |
| 66944119 | 66914574 | TCAAAATGACCAGACCCTGAAGAAAGGCTG | |
| chrX: 66763874- | chrX: 66913375- | GTAAAATATTTTCTTTTTATACTGAACACATGCATAGACAACTTACCTGAGCAAGCTGCTTTTTGGAGACATTTGCACATCTTTTGGGAT | 1207 |
| 66944119 | 66913494 | CACGTTGTTAAGAAGTAGAACTAAGGGAAA | |
| chrX: 66763874- | chrX: 66914035- | TCTGAGTAATAATGCTTATAGTATTATGCAATTGTTTGGCTGCTGCAAGAAATTCAGCAGACTCCAACAAGTAGTCTTTCTTGGTCTCTG | 1208 |
| 66944119 | 66914154 | AGTGACTGTAACTTAAATTCTACCTCCCTT | |
| chrX: 66763874- | chrX: 66914215- | CTATGTTCAGAGAGATGCACGCACACATATATATGTATATATATAGTATATTTGTCAATAAAGCAGAAAAGAAGAAAAAACTCCAAGTAA | 1209 |
| 66944119 | 66914334 | ACAATTTTCCATTTCCCCATCTCACTTCTG | |
| chrX: 66763874- | chrX: 66914095- | ACTCCAACAAGTAGTCTTTCTTGGTCTCTGAGTGACTGTAACTTAAATTCTACCTCCCTTCTCTTCTCCTACATCTTCTCACTCCCCACC | 1210 |
| 66944119 | 66914214 | CCACCCCCACATACACACAATTCTTGTCCA | |
| chrX: 66763874- | chrX: 66913855- | TGCATCTTGACCAAGATGAAAAGTCACCAGCCCAAGCTTGTGCAGTGAAGTGTCATGTTGGCCACAATGAAACTGAAAGAGACTGATGAC | 1211 |
| 66944119 | 66913974 | TCTCCTCAGGGTGGAAAATGAGGCATGGAA | |
| chrX: 66763874- | chrX: 66914275- | AAGCAGAAAAGAAGAAAAAACTCCAAGTAAACAATTTTCCATTTCCCCATCTCACTTCTGTCTTACAAGTGGATAGGAAAAGAAAAACCC | 1212 |
| 66944119 | 66914394 | CCAGTAAAAAATGGCAACCGCCCACCTCCC | |
| chrX: 66763874- | chrX: 66914575- | ACTTGCCTCATTCAAAATGAGGGCTCTAGAGGGCTCTAGTGGATAGTCTGGAGAAACCTGGCGTCTGAGGCTTAGGAGCTTAGGTTTTTG | 1213 |
| 66944119 | 66914694 | CTCCTCAACACAGACTTTGACGTTGGGGTT | |
| chrX: 66763874- | chrX: 66913675- | CAAGGCATAGAAATATATTTTCCCTTGCTTAATACCCCATGGAACCAATGCCCCTGTGGTTGAAGTAAAAATTGATTGTTGAGGGACATT | 1214 |
| 66944119 | 66913794 | TCAGCCCTCTAGCAGTCAACAATTAAAAAC | |
| chrX: 66763874- | chrX: 66915297- | ACTCCTTGATTGCTCTCTCACATCACATGCTTCTCTTCATCAGTTGTAAGCCTCTCATTCTTCTCCCAAGCCAGACTCAAATATTGTATT | 1215 |
| 66944119 | 66915416 | GATGTCAAAGAAGAATCACTTAGAGTTTGG | |
| chrX: 66763874- | chrX: 66915237- | AAACACACTGAGAGACTACAGTCCGACTTTCCCTCTTACATCTAGCCTTACTGTAGCCACACTCCTTGATTGCTCTCTCACATCACATGC | 1216 |
| 66944119 | 66915356 | TTCTCTTCATCAGTTGTAAGCCTCTCATTC | |
| chrX: 66763874- | chrX: 66915177- | AGTAAACAAGGACCAGATTTCTGCTTCTCCAGGAGAAGAAGCCAGCCAACCCCTCTCTTCAAACACACTGAGAGACTACAGTCCGACTTT | 1217 |
| 66944119 | 66915296 | CCCTCTTACATCTAGCCTTACTGTAGCCAC | |
| chrX: 66763874- | chrX: 66915117- | TAGATCTTAGCCTCAGGCCCTGTCACTGAGCTGAAGGTAGTAGCTGATCCACAGAAGTTCAGTAAACAAGGACCAGATTTCTGCTTCTCC | 1218 |
| 66944119 | 66915236 | AGGAGAAGAAGCCAGCCAACCCCTCTCTTC | |
| chrX: 66763874- | chrX: 66915357- | TTCTCCCAAGCCAGACTCAAATATTGTATTGATGTCAAAGAAGAATCACTTAGAGTTTGGAATATCTTGTTCTCTCTCTGCTCCATAGCT | 1219 |
| 66944119 | 66915476 | TCCATATTGACACCAGTTTCTTTCTAGTGG | |
| chrX: 66763874- | chrX: 66914877- | CTTGATCACATATGATGGGGGCCAGGCACTGACTCAGGCGGATGCAGTGAAGCTCTGGCTCAGTCGCTTGCTTTTCGTGGTGTGCTGCCA | 1220 |
| 66944119 | 66914996 | GGAAGAAACTTTGCTGATGGGACTCAAGGT | |
| chrX: 66763874- | chrX: 66914817- | TATCATGATTATTGTTTCCTGTAATGTGGCTTGGCATTGGCAAAGTGCTTTTTGATTGTTCTTGATCACATATGATGGGGGCCAGGCACT | 1221 |
| 66944119 | 66914936 | GACTCAGGCGGATGCAGTGAAGCTCTGGCT | |
| chrX: 66763874- | chrX: 66914757- | agggatgttgtgaggaCGGGCTGTAGAAGTAATAGTGGTTACCATTCATGTAGTTGTGAGTATCATGATTATTGTTTCCTGTAATGTGGC | 1222 |
| 66944119 | 66914876 | TTGGCATTGGCAAAGTGCTTTTTGATTGTT | |
| chrX: 66763874- | chrX: 66914937- | CAGTCGCTTGCTTTTCGTGGTGTGCTGCCAGGAAGAAACTTTGCTGATGGGACTCAAGGTGTCACCTTGGACAAGAAGCAACTGTGTCTG | 1223 |
| 66944119 | 66915056 | TCTGAGGTTCCTGTGGCCATCTTTATTTGT | |
| chrX: 66763874- | chrX: 66914997- | GTCACCTTGGACAAGAAGCAACTGTGTCTGTCTGAGGTTCCTGTGGCCATCTTTATTTGTGTATTAGGCAATTCGTATTTCCCCCTTAGG | 1224 |
| 66944119 | 66915116 | TTCTAGCCTTCTGGATCCCAGCCAGTGACC | |
| chrX: 66763874- | chrX: 66915477- | AGAAGTGGAGTCTGTGAAGCCAGGGAAACACACATGTGAGAGTCAGAAGGACTCTCCCTGACTTGCCTGGGGCCTGTCTTTCCCACCTTC | 1225 |
| 66944119 | 66915596 | TCCAGTCTGTCTAAacacacacacacacac | |
| chrX: 66763874- | chrX: 66915057- | GTATTAGGCAATTCGTATTTCCCCCTTAGGTTCTAGCCTTCTGGATCCCAGCCAGTGACCTAGATCTTAGCCTCAGGCCCTGTCACTGAG | 1226 |
| 66944119 | 66915176 | CTGAAGGTAGTAGCTGATCCACAGAAGTTC | |
| chrX: 66763874- | chrX: 66915417- | AATATCTTGTTCTCTCTCTGCTCCATAGCTTCCATATTGACACCAGTTTCTTTCTAGTGGAGAAGTGGAGTCTGTGAAGCCAGGGAAACA | 1227 |
| 66944119 | 66915536 | CACATGTGAGAGTCAGAAGGACTCTCCCTG | |
| chrX: 66763874- | chrX: 66915839- | TTTTAACTACTTTCTATTTGTTTGAATGTTGCATATTTCTACTAGTGAAATTTTCCCTTAATAAAGCCATTAATACACCAATCGTATTTT | 1228 |
| 66944119 | 66915958 | CTTATTTACAACAGACTGAGAGAATTAATG | |
| chrX: 66763874- | chrX: 66915719- | TCTGGAGAAAAGCCAAGGAAGGACTTCAGGAGGGGAGTTTCCCCCTTCTCAGGGCAGAATTTTAATCTCCAGACCAACAAGAAGTTCCCT | 1229 |
| 66944119 | 66915838 | AATGTGGATTGAAAGGCTAATGAGGTTTAT | |
| chrX: 66763874- | chrX: 66915779- | TTTAATCTCCAGACCAACAAGAAGTTCCCTAATGTGGATTGAAAGGCTAATGAGGTTTATTTTTAACTACTTTCTATTTGTTTGAATGTT | 1230 |
| 66944119 | 66915898 | GCATATTTCTACTAGTGAAATTTTCCCTTA | |
| chrX: 66763874- | chrX: 66916224- | GTATCTGGACAGTGAGCCCCTTTCTTCTGGCTCAGTAGTCAGAGAGAGGAGACTTGGAGACAGTTTCTGCTGGATCCTGTGCTTTGGCAA | 1231 |
| 66944119 | 66916343 | GGATGTGCAGCATTGCATATCATTCTATCA | |
| chrX: 66763874- | chrX: 66916584- | CCAAGGATTGGCTCTGCTCCCTGCTTCTCATCCCTGTCCTAGTTCTTCCTCACCTATCTCCATTTCCCACTACTGATCCTTCTCTCCAGT | 1232 |
| 66944119 | 66916703 | AAGATGCTATTcaacccgatgaaatataaa | |
| chrX: 66763874- | chrX: 66916164- | GGCTCTGCAATTTCATATGAGCAGGTTTTTGGTAAAATCTTTTGTCCCTCACTCAGGGTGGTATCTGGACAGTGAGCCCCTTTCTTCTGG | 1233 |
| 66944119 | 66916283 | CTCAGTAGTCAGAGAGAGGAGACTTGGAGA | |
| chrX: 66763874- | chrX: 66916464- | TGGGAGGGGATTGGAGACTTGGGGGAAAGAATCAAGGAGCCTTCTTGCCTGGGGGAATTTGGCATGCACTTATTAATCCCATTTGGTTGC | 1234 |
| 66944119 | 66916583 | ACTCCCTACTAATCCCTCACTCCATACCTG | |
| chrX: 66763874- | chrX: 66916344- | TTAATTATGTTTACTCCTCCATGAACTAAAAACCATTAGACTAAATAGTCCAACATAAACCTTGAAAGATAAAATTTGATATTCTTTTGC | 1235 |
| 66944119 | 66916463 | CTGGCCATTTCTCTGACCCAGAATTGGGGC | |
| chrX: 66763874- | chrX: 66916524- | GGCATGCACTTATTAATCCCATTTGGTTGCACTCCCTACTAATCCCTCACTCCATACCTGCCAAGGATTGGCTCTGCTCCCTGCTTCTCA | 1236 |
| 66944119 | 66916643 | TCCCTGTCCTAGTTCTTCCTCACCTATCTC | |
| chrX: 66763874- | chrX: 66916044- | TTGGACTGTTTCACATTTGTTTTTAATGTCAGTTTAAATGTAATTGTAAAAGCATGTATGCTCTAAAATCATGTAGTTACTTTTTTCAGT | 1237 |
| 66944119 | 66916163 | GGAAAAGCCTGGTATTCGAAAGCATTTCCA | |
| chrX: 66763874- | chrX: 66916284- | CAGTTTCTGCTGGATCCTGTGCTTTGGCAAGGATGTGCAGCATTGCATATCATTCTATCATTAATTATGTTTACTCCTCCATGAACTAAA | 1238 |
| 66944119 | 66916403 | AACCATTAGACTAAATAGTCCAACATAAAC | |
| chrX: 66763874- | chrX: 66916104- | CTCTAAAATCATGTAGTTACTTTTTTCAGTGGAAAAGCCTGGTATTCGAAAGCATTTCCAGGCTCTGCAATTTCATATGAGCAGGTTTTT | 1239 |
| 66944119 | 66916223 | GGTAAAATCTTTTGTCCCTCACTCAGGGTG | |
| chrX: 66763874- | chrX: 66916404- | CTTGAAAGATAAAATTTGATATTCTTTTGCCTGGCCATTTCTCTGACCCAGAATTGGGGCTGGGAGGGGATTGGAGACTTGGGGGAAAGA | 1240 |
| 66944119 | 66916523 | ATCAAGGAGCCTTCTTGCCTGGGGGAATTT | |
| chrX: 66763874- | chrX: 66917133- | CAGAAAGTTCACCTGCTTGGGGTAAAGGTCATGAAGTGGAGAATGTGGGGCTCAGTAACTAGCAATAGTAAAAAACATCATTGATTGGCT | 1241 |
| 66944119 | 66917252 | TGCAGAATTTACTctgttctaagcatctta | |
| chrX: 66763874- | chrX: 66917073- | CTCTCTTATGTTACATTTCAGTCCTTGTGAAACTCTATATGTTTCATCAGTTCACTTTTTCAGAAAGTTCACCTGCTTGGGGTAAAGGTC | 1242 |
| 66944119 | 66917192 | ATGAAGTGGAGAATGTGGGGCTCAGTAACT | |
| chrX: 66763874- | chrX: 66917013- | ctctcaggaaccctcgaAAAAACTTATAGGACTTATAGGACTGTTGGGGATCTGCCAAGTCTCTCTTATGTTACATTTCAGTCCTTGTGA | 1243 |
| 66944119 | 66917132 | AACTCTATATGTTTCATCAGTTCACTTTTT | |
| chrX: 66763874- | chrX: 66917327- | CCAGCATGACTCTCAATCTTTGACTTGAGACCAGTTGCCCAACATGGAAGGTTATACTTTTCACAGTTTACCACCATAAGCAGTCTTTCA | 1244 |
| 66944119 | 66917446 | GAGTGATTTCTAGCTAGAGATCCATTCTTA | |
| chrX: 66763874- | chrX: 66917660- | TAATAAACCTTTTATGTGAAAAGCATTTTAGAACTTCAGTGTCATTATTGCATTCTGCCTCCTGGAGTTCAGTGCACTTTTTCACCATGC | 1245 |
| 66944119 | 66917779 | TTTAATCTTGGAGTCCTGGTGGTACAGAAT | |
| chrX: 66763874- | chrX: 66918053- | gagctCCCCTGACTCACAATCCAGTATTCCTCTGACCTTCTAATCCTAAAGTTATACAGTAAGGTCCCTTGACTCTAATCCTAGTAGATG | 1246 |
| 66944119 | 66918172 | GAAAGATGGCTGGCATGATTTAAGCcagag | |
| chrX: 66763874- | chrX: 66918380- | TTTAGATTTAAGCATTGCAATATATAAGCACTGCACACATGCATTCACAAAAGTATAGCCTAGTCTAGCTTCACAAAGAATTTGTAGCCC | 1247 |
| 66944119 | 66918499 | TACACCAAACACACCTTTATGTTTACTTAG | |
| chrX: 66763874- | chrX: 66918440- | TAGTCTAGCTTCACAAAGAATTTGTAGCCCTACACCAAACACACCTTTATGTTTACTTAGTGTTTAGAATTAGATTTAAGATCAGAATTT | 1248 |
| 66944119 | 66918559 | AGTTTCACAGGCATTCATGTGTGGAAGAAC | |
| chrX: 66763874- | chrX: 66918620- | CCCTGTCAATCCTGCTTTCTGCCATTCTTCATGCCTGAGTTAGGGCCCCTGCAAGCCATTCACTGGTTAATCTTTAGGAATGAATGGAGA | 1249 |
| 66944119 | 66918739 | GTGAAAACCAGTTTGGAGGGTTCACTGTGT | |
| chrX: 66763874- | chrX: 66918500- | TGTTTAGAATTAGATTTAAGATCAGAATTTAGTTTCACAGGCATTCATGTGTGGAAGAACCTCAGTTATTGTTTTTTGTTTCATACTGTC | 1250 |
| 66944119 | 66918619 | TCACCCTTGCTTTCCCTGCTGTGTCTGGAC | |
| chrX: 66763874- | chrX: 66918560- | CTCAGTTATTGTTTTTTGTTTCATACTGTCTCACCCTTGCTTTCCCTGCTGTGTCTGGACCCCTGTCAATCCTGCTTTCTGCCATTCTTC | 1251 |
| 66944119 | 66918679 | ATGCCTGAGTTAGGGCCCCTGCAAGCCATT | |
| chrX: 66763874- | chrX: 66920392- | AGAGAAGCCAGAACACCTGGTGCAGCTAGGGCCACTGTGGTCACAGGGACAAGCACACTACCTGGGTCCTGGAGGCAAGTGGGAATGCAG | 1252 |
| 66944119 | 66920511 | TTTTTCTTCCTTAAGCAGATGCCATATAGG | |
| chrX: 66763874- | chrX: 66920272- | CCAGGATTCCAAGTCGTCTAGCAACATCCTGGTCTCTGCTGCAGACAGAACAGAGGATCCCCCGGCAGAATGAATGGAGTCTGATTTCAA | 1253 |
| 66944119 | 66920391 | TTACGTTCAGTATAGTCACTCTCTTTAGGC | |
| chrX: 66763874- | chrX: 66919972- | ATGGCCCTTTCTTGTCACTGCTCTGACCCAAGACTAACAGGGCAGAGATAGTGAACTCACATACTATTAAAACTATCCACTTATACTTCC | 1254 |
| 66944119 | 66920091 | CCCTTTCTCTTTGCTTTATCACTCCATTTA | |
| chrX: 66763874- | chrX: 66919912- | TATCAGGCCAGGTGTTACAATCTCCTAAAGAGGAGGTATGGACTGGAAAGCCCCTTGCCAATGGCCCTTTCTTGTCACTGCTCTGACCCA | 1255 |
| 66944119 | 66920031 | AGACTAACAGGGCAGAGATAGTGAACTCAC | |
| chrX: 66763874- | chrX: 66919852- | CAGGGGTCTAATGCCCCAGGGCTGAAAAGTTAGTTCCCCATAGGATCCATCCAGGCATGATATCAGGCCAGGTGTTACAATCTCCTAAAG | 1256 |
| 66944119 | 66919971 | AGGAGGTATGGACTGGAAAGCCCCTTGCCA | |
| chrX: 66763874- | chrX: 66920092- | AGTAAACCAATGAGTCTCTGCCTTGACACAGTGGCAAGCTGACCTGTATCTTATATGAAAGAATTAGATTTGACTCTGGGGCTCAGGTGC | 1257 |
| 66944119 | 66920211 | AGAGGGCAGGAGGGGCATAAGGATGGCCTT | |
| chrX: 66763874- | chrX: 66920452- | CCTGGGTCCTGGAGGCAAGTGGGAATGCAGTTTTTCTTCCTTAAGCAGATGCCATATAGGCCTGGGGAGGAGGATGTGAGAATACCAGCC | 1258 |
| 66944119 | 66920571 | AAGTTCTCATTGGCACTATACAGAGAAAGG | |
| chrX: 66763874- | chrX: 66919672- | TTTTCCTTAATCATGGTTGAGAAGGCCTATATCTTGGAGTGGCCAGGAGTGAGACTGGAACAGTACCTAAAGGTTAAGGACGCTAAAGAA | 1259 |
| 66944119 | 66919791 | GTTACAGATTGGTTACATCTGCTCCTCCCT | |
| chrX: 66763874- | chrX: 66919792- | AGGAATGATCCATGGAACCTGATTTGAAATTTTTTTCTCTGGTGCTATAGATAGCTCCCACAGGGGTCTAATGCCCCAGGGCTGAAAAGT | 1260 |
| 66944119 | 66919911 | TAGTTCCCCATAGGATCCATCCAGGCATGA | |
| chrX: 66763874- | chrX: 66920572- | GGAATTATTTCATCTTGATGGATTCTCCCCACAGTCTCTGCACATATTGATCTTACTTGTAATGAGTTTGCTTAGGTTCACGAGTCATCA | 1261 |
| 66944119 | 66920691 | TCCCAGGGAGATCTGAGTCATTGGTGGGAA | |
| chrX: 66763874- | chrX: 66920512- | CCTGGGGAGGAGGATGTGAGAATACCAGCCAAGTTCTCATTGGCACTATACAGAGAAAGGGGAATTATTTCATCTTGATGGATTCTCCCC | 1262 |
| 66944119 | 66920631 | ACAGTCTCTGCACATATTGATCTTACTTGT | |
| chrX: 66763874- | chrX: 66919612- | GACCTTAGGTAAAGGGATTCAAACTTCCTAAGACTTTGGAAACTTCACGCCACTTTCACCTTTTCCTTAATCATGGTTGAGAAGGCCTAT | 1263 |
| 66944119 | 66919731 | ATCTTGGAGTGGCCAGGAGTGAGACTGGAA | |
| chrX: 66763874- | chrX: 66920212- | CATGGAAGAAAAGAAGTCCTTGGATACTGAGTAACAGCTGAGACTAGCAAGCCTCATTGTCCAGGATTCCAAGTCGTCTAGCAACATCCT | 1264 |
| 66944119 | 66920331 | GGTCTCTGCTGCAGACAGAACAGAGGATCC | |
| chrX: 66763874- | chrX: 66919732- | CAGTACCTAAAGGTTAAGGACGCTAAAGAAGTTACAGATTGGTTACATCTGCTCCTCCCTAGGAATGATCCATGGAACCTGATTTGAAAT | 1265 |
| 66944119 | 66919851 | TTTTTTCTCTGGTGCTATAGATAGCTCCCA | |
| chrX: 66763874- | chrX: 66920632- | AATGAGTTTGCTTAGGTTCACGAGTCATCATCCCAGGGAGATCTGAGTCATTGGTGGGAAAGTCGAGGCGACAGATTATATCTCACTGAT | 1266 |
| 66944119 | 66920751 | CTCACTGTCACCAATTGCTCTGTGTGTCCC | |
| chrX: 66763874- | chrX: 66920152- | GAATTAGATTTGACTCTGGGGCTCAGGTGCAGAGGGCAGGAGGGGCATAAGGATGGCCTTCATGGAAGAAAAGAAGTCCTTGGATACTGA | 1267 |
| 66944119 | 66920271 | GTAACAGCTGAGACTAGCAAGCCTCATTGT | |
| chrX: 66763874- | chrX: 66920692- | AGTCGAGGCGACAGATTATATCTCACTGATCTCACTGTCACCAATTGCTCTGTGTGTCCCTCCACCTTTTGAAAAAGTCCATGGATTCAT | 1268 |
| 66944119 | 66920811 | TTGTGTGTAATTCATTTGGATTTATTTCTT | |
| chrX: 66763874- | chrX: 66920032- | ATACTATTAAAACTATCCACTTATACTTCCCCCTTTCTCTTTGCTTTATCACTCCATTTAAGTAAACCAATGAGTCTCTGCCTTGACACA | 1269 |
| 66944119 | 66920151 | GTGGCAAGCTGACCTGTATCTTATATGAAA | |
| chrX: 66763874- | chrX: 66920752- | TCCACCTTTTGAAAAAGTCCATGGATTCATTTGTGTGTAATTCATTTGGATTTATTTCTTCTTTATCAATAGCTTTAGTGGGGTATTGCA | 1270 |
| 66944119 | 66920871 | AATGGGAAAGTTGCCCCAGAGAACAGTGTA | |
| chrX: 66763874- | chrX: 66920332- | CCCGGCAGAATGAATGGAGTCTGATTTCAATTACGTTCAGTATAGTCACTCTCTTTAGGCAGAGAAGCCAGAACACCTGGTGCAGCTAGG | 1271 |
| 66944119 | 66920451 | GCCACTGTGGTCACAGGGACAAGCACACTA | |
| chrX: 66763874- | chrX: 66919552- | GATTCTTTTCATGTGTCAAATTTCATAGTGAGATAGGGAGAACAGAAACATCACATCCTTGACCTTAGGTAAAGGGATTCAAACTTCCTA | 1272 |
| 66944119 | 66919671 | AGACTTTGGAAACTTCACGCCACTTTCACC | |
| chrX: 66763874- | chrX: 66921370- | ATTTTGGGATACACAAGTTTCAGGAATTTTTTAAAAATCTATTAATGCCTTCTAGGTGTGTGTATGCACGCTTGCAGACATGTGCCCATG | 1273 |
| 66944119 | 66921489 | CACAAGCATGGGAAGGCAGTAAGGCATTCA | |
| chrX: 66763874- | chrX: 66921769- | GTTTGGAGTCAAGGGTGAAGAAGGGAGGCAGGGATGATATAACCCCAGCCCCACTCCTCAACTCTGCTTTTGAGTTAGAAGTAGGGTTCA | 1274 |
| 66944119 | 66921888 | GGGCTTCAGATTCCTTGGGGAGgcagtaga | |
| chrX: 66763874- | chrX: 66921649- | gtcatgctGAAAATCTTTAAGCCACTATAGTGTCCCAAATCTATTCCAGTTTGGGCAGATGACTGGAGTATTCTCATAGCCTCCTGTCTA | 1275 |
| 66944119 | 66921768 | TTCCCTTCTGGATTTGATACTAGTTATGAA | |
| chrX: 66763874- | chrX: 66921709- | GACTGGAGTATTCTCATAGCCTCCTGTCTATTCCCTTCTGGATTTGATACTAGTTATGAAGTTTGGAGTCAAGGGTGAAGAAGGGAGGCA | 1276 |
| 66944119 | 66921828 | GGGATGATATAACCCCAGCCCCACTCCTCA | |
| chrX: 66763874- | chrX: 66922124- | CAAAGCTAGAAAGGTCCAGTAATGGGGAAGATGGGGTCTTTCTGTAGGAACTGTAGCAGGGGAGCAGATCCTGTAGGCCACCAGTCTGTG | 1277 |
| 66944119 | 66922243 | GAGCTGTGTCCAAGAACTCATGTTTGCAAT | |
| chrX: 66763874- | chrX: 66922244- | AAGCCCACCAAATGACAAGTTATTGTGGGGTTCAGGCCTCTAACTCAAGAAGATGGTCTTGGCCCAGATCATACCTTGCAGCCTGTGCCT | 1278 |
| 66944119 | 66922363 | TTGGTGGGATGTGGGTGTTGGCAGTGGCTA | |
| chrX: 66763874- | chrX: 66922364- | TGCATATCTCCTTATTACTGGCTGTGCCAAAGCCCCGCAGAAATGATTGTTGGACAAAGTCATCTTGCACTCAGGGCTGGTTTTCCAGGC | 1279 |
| 66944119 | 66922483 | TTCCTTGTTATTTTCCCCTGAGTTCTTCTG | |
| chrX: 66763874- | chrX: 66922304- | GGCCCAGATCATACCTTGCAGCCTGTGCCTTTGGTGGGATGTGGGTGTTGGCAGTGGCTATGCATATCTCCTTATTACTGGCTGTGCCAA | 1280 |
| 66944119 | 66922423 | AGCCCCGCAGAAATGATTGTTGGACAAAGT | |
| chrX: 66763874- | chrX: 66922484- | TGTTCCTCTTGCAACACCAACCCCACTATTTTCCTCTTCCCTACCCTAGTTGTTGGTCCAAACATGTAATCCATTCTTGCAGTGATTTAT | 1281 |
| 66944119 | 66922603 | TGGGTGACACCATGACTGGAGTTTGCATTG | |
| chrX: 66763874- | chrX: 66922424- | CATCTTGCACTCAGGGCTGGTTTTCCAGGCTTCCTTGTTATTTTCCCCTGAGTTCTTCTGTGTTCCTCTTGCAACACCAACCCCACTATT | 1282 |
| 66944119 | 66922543 | TTCCTCTTCCCTACCCTAGTTGTTGGTCCA | |
| chrX: 66763874- | chrX: 66922184- | GGAGCAGATCCTGTAGGCCACCAGTCTGTGGAGCTGTGTCCAAGAACTCATGTTTGCAATAAGCCCACCAAATGACAAGTTATTGTGGGG | 1283 |
| 66944119 | 66922303 | TTCAGGCCTCTAACTCAAGAAGATGGTCTT | |
| chrX: 66763874- | chrX: 66923094- | TGGTTCATGGTCTTTCTCAGTGCAACTGCTTATGCTAGACCTCAGAATTATGACCTTTTCAATTATTTATATTTCTGTCTATATAAATAC | 1284 |
| 66944119 | 66923213 | TGGAAAAAATAGTACAAAGTAAGCATCGGA | |
| chrX: 66763874- | chrX: 66923274- | GAGTTTTGGATTATTGTGGTTGTCTTAAATAATGTTATTTCTATCATTCTTTCCAATGACTGTCTCCTAGCATAGTTCCCATTTTACAGA | 1285 |
| 66944119 | 66923393 | CTGATGGCAGAGGCAGAAAGATTCTCTCAC | |
| chrX: 66763874- | chrX: 66923214- | ATGCCTAAGGACCTCTAAATTGTGTGTGTGAGCACATGGGGAAGATGGTTCTTAAGGTTTGAGTTTTGGATTATTGTGGTTGTCTTAAAT | 1286 |
| 66944119 | 66923333 | AATGTTATTTCTATCATTCTTTCCAATGAC | |
| chrX: 66763874- | chrX: 66923034- | AAATTCTCTCTTTGGTTTCTACCTTTCTTATTTATTATTATTATGTTAAAGGGATTAAAGTGGTTCATGGTCTTTCTCAGTGCAACTGCT | 1287 |
| 66944119 | 66923153 | TATGCTAGACCTCAGAATTATGACCTTTTC | |
| chrX: 66763874- | chrX: 66923334- | TGTCTCCTAGCATAGTTCCCATTTTACAGACTGATGGCAGAGGCAGAAAGATTCTCTCACTTCTTTGATACTATTGAGGACTTCAGCCTT | 1288 |
| 66944119 | 66923453 | TCACCGCTCTTCTCCCCTTTGCTAAAAAAG | |
| chrX: 66763874- | chrX: 66922974- | AGGGGCCAGACACAGATGAGAGCAGGTTTTGTTGTATTTATCCATTTAAATTGAGCAATAAAATTCTCTCTTTGGTTTCTACCTTTCTTA | 1289 |
| 66944119 | 66923093 | TTTATTATTATTATGTTAAAGGGATTAAAG | |
| chrX: 66763874- | chrX: 66923154- | AATTATTTATATTTCTGTCTATATAAATACTGGAAAAAATAGTACAAAGTAAGCATCGGAATGCCTAAGGACCTCTAAATTGTGTGTGTG | 1290 |
| 66944119 | 66923273 | AGCACATGGGGAAGATGGTTCTTAAGGTTT | |
| chrX: 66763874- | chrX: 66930011- | CCCAAAGAACAAGTCTAGTCATTCTCCATTTCTAGTCTCTTTCCCTAGCAATCGGCTAGACATGCTAGACATAGACACATGTACATCACT | 1291 |
| 66944119 | 66930130 | CCTTTGAATTACAACATTCAGTATTTGTCT | |
| chrX: 66763874- | chrX: 66931354- | GAAGGCTATGAATGTCAGCCCATCTTTCTGAATGTCCTGGAAGCCATTGAGCCAGGTGTAGTGTGTGCTGGACACGACAACAACCAGCCC | 1292 |
| 66944119 | 66931473 | GACTCCTTTGCAGCCTTGCTCTCTAGCCTC | |
| chrX: 66763874- | chrX: 66930994- | TAGGTTGGTCTAGCACAAGAATCAGAGTTTTCCTCTGCAAGCTATGAAAAATTTGGGTTTAGCAGGTATTTGGGATGATGAATTATACAT | 1293 |
| 66944119 | 66931113 | TTAACCAGTGTTGAATGAGCACTTGTCCTT | |
| chrX: 66763874- | chrX: 66931174- | TAAAAAGGTAGTTGCATTGTGTGTTTTTGACCACTGATGATAAATTCAAGTCTCTCTTCCTTCCCAATAGCCCGGAAGCTGAAGAAACTT | 1294 |
| 66944119 | 66931293 | GGTAATCTGAAACTACAGGAGGAAGGAGAG | |
| chrX: 66763874- | chrX: 66931414- | GTGTGTGCTGGACACGACAACAACCAGCCCGACTCCTTTGCAGCCTTGCTCTCTAGCCTCAATGAACTGGGAGAGAGACAGCTTGTACAC | 1295 |
| 66944119 | 66931533 | GTGGTCAAGTGGGCCAAGGCCTTGCCTGGT | |
| chrX: 66763874- | chrX: 66930934- | AAAGTTCCAGGTATGAATACTGAAGGCTGCATTCAGGCAGAGCTGGATCCAAGGATATGCTAGGTTGGTCTAGCACAAGAATCAGAGTTT | 1296 |
| 66944119 | 66931053 | TCCTCTGCAAGCTATGAAAAATTTGGGTTT | |
| chrX: 66763874- | chrX: 66931294- | GCTTCCAGCACCACCAGCCCCACTGAGGAGACAACCCAGAAGCTGACAGTGTCACACATTGAAGGCTATGAATGTCAGCCCATCTTTCTG | 1297 |
| 66944119 | 66931413 | AATGTCCTGGAAGCCATTGAGCCAGGTGTA | |
| chrX: 66763874- | chrX: 66931234- | TTCCCAATAGCCCGGAAGCTGAAGAAACTTGGTAATCTGAAACTACAGGAGGAAGGAGAGGCTTCCAGCACCACCAGCCCCACTGAGGAG | 1298 |
| 66944119 | 66931353 | ACAACCCAGAAGCTGACAGTGTCACACATT | |
| chrX: 66763874- | chrX: 66931114- | AAGGAGTTTAGAGTCTGTGACCAGGGAGAATGGTGATTTTCTTAGCTAGGGCAGTTTTTCTAAAAAGGTAGTTGCATTGTGTGTTTTTGA | 1299 |
| 66944119 | 66931233 | CCACTGATGATAAATTCAAGTCTCTCTTCC | |
| chrX: 66763874- | chrX: 66931054- | AGCAGGTATTTGGGATGATGAATTATACATTTAACCAGTGTTGAATGAGCACTTGTCCTTAAGGAGTTTAGAGTCTGTGACCAGGGAGAA | 1300 |
| 66944119 | 66931173 | TGGTGATTTTCTTAGCTAGGGCAGTTTTTC | |
| chrX: 66763874- | chrX: 66931840- | TACCCATATATGCCTTAGGATGCTCTTCTATATTTGCACACACAGGCTCACCCCAAAGATAATCTCTAGTTTGACTGACATTCTGTCTTC | 1301 |
| 66944119 | 66931959 | AATGTCATCTTTAGGAGCTATATCATGGGA | |
| chrX: 66763874- | chrX: 66931720- | AGAAAGATAGTATTATCGGGTGTCTTATGTGGCCCACATTGATGCACAGCAGTCATGCTTTCATATTCAACTCACAAAAATGGTCAGCAA | 1302 |
| 66944119 | 66931839 | ATTTTCCATTAATCACAAATCACATAGACA | |
| chrX: 66763874- | chrX: 66931780- | TCATATTCAACTCACAAAAATGGTCAGCAAATTTTCCATTAATCACAAATCACATAGACATACCCATATATGCCTTAGGATGCTCTTCTA | 1303 |
| 66944119 | 66931899 | TATTTGCACACACAGGCTCACCCCAAAGAT | |
| chrX: 66763874- | chrX: 66932115- | AAATTGATGGTTTCCAGTTTTTGAAAAAAGGAACGCTTTTTGCACCTTAAACTACCTAAGGAATCATAATGAGAGGAAAGATTAGGTAAT | 1304 |
| 66944119 | 66932234 | AGTGAAAGAATTACCAAGTGTTGGTCTAAC | |
| chrX: 66763874- | chrX: 66932561- | agtgAAGTATATGCCCTTTAAAGGTTCTCCTAATCCTGCAATTATGATTCAAAGATTCTTTTGAAATAACAACAACCAAACCTTCTCTTG | 1305 |
| 66944119 | 66932680 | TGGAGTCAAAGATTAACCTGCCTTTCaata | |
| chrX: 66763874- | chrX: 66933126- | GGTCGACTAGCCAATAAGAACACTGGGAAGGAAACCCAAGGACTCTGACTGGATATGCTCTGTGCCAAAACAGAGGGTTCACTCAGAGAG | 1306 |
| 66944119 | 66933245 | GAAAAATATAAAAAAGAAAAAGGAGAAGGT | |
| chrX: 66763874- | chrX: 66933006- | GGCTTGTTGCATTTGAAGACATTTCATGTTGGCTGTCAAGTCTTAGATTTGTATTTCCAACTCACAGGGCCTGGTCACAGCCCTAACCAT | 1307 |
| 66944119 | 66933125 | CTCTTATACCTTCTCAGCTTGGGAAGCTGA | |
| chrX: 66763874- | chrX: 66932946- | GCTCTGGGCTTTACAGTGAGGGGCCAAGGCTTCATTGAAGGCCACTTGGGTCATAGTATGGGCTTGTTGCATTTGAAGACATTTCATGTT | 1308 |
| 66944119 | 66933065 | GGCTGTCAAGTCTTAGATTTGTATTTCCAA | |
| chrX: 66763874- | chrX: 66933066- | CTCACAGGGCCTGGTCACAGCCCTAACCATCTCTTATACCTTCTCAGCTTGGGAAGCTGAGGTCGACTAGCCAATAAGAACACTGGGAAG | 1309 |
| 66944119 | 66933185 | GAAACCCAAGGACTCTGACTGGATATGCTC | |
| chrX: 66763874- | chrX: 66933186- | TGTGCCAAAACAGAGGGTTCACTCAGAGAGGAAAAATATAAAAAAGAAAAAGGAGAAGGTTGCTTTAATTCTTATCACTTTTTCATCTGG | 1310 |
| 66944119 | 66933305 | ATATTTTGATATCATGTGTTTGACAGAGAT | |
| chrX: 66763874- | chrX: 66933600- | GCTTTCCTTGGCCCAACCACCAAGATGGAAAGCCCCCTCCCCTTACATTAACAAATCTGCAAGCCAATATCAGTTCACCATCTAGCTTGC | 1311 |
| 66944119 | 66933719 | CAGACTAAATGATTTCTGACCCCAAGTCTT | |
| chrX: 66763874- | chrX: 66933540- | CTTGACAATAACCTAGTGCAAAACACTATAGCAGAATTTGTATGACTTGGGATCACTGGGGCTTTCCTTGGCCCAACCACCAAGATGGAA | 1312 |
| 66944119 | 66933659 | AGCCCCCTCCCCTTACATTAACAAATCTGC | |
| chrX: 66763874- | chrX: 66933720- | TTAAAAGAATAGCTTCAAAAGAAAGCCAATTACCACATTCACAAGAACTGTTCTTCATATTATCTATAATTACCTACAAGTACAAGTAAT | 1313 |
| 66944119 | 66933839 | TTGCTAATTCAATAGATTGAGTTCTTGACC | |
| chrX: 66763874- | chrX: 66933780- | TATCTATAATTACCTACAAGTACAAGTAATTTGCTAATTCAATAGATTGAGTTCTTGACCTGTAAGATGAACTGTGCTAGGCCCCTAATA | 1314 |
| 66944119 | 66933899 | AGATAAATTTTGTTTTAAGTTTTCTGTGAC | |
| chrX: 66763874- | chrX: 66933660- | AAGCCAATATCAGTTCACCATCTAGCTTGCCAGACTAAATGATTTCTGACCCCAAGTCTTTTAAAAGAATAGCTTCAAAAGAAAGCCAAT | 1315 |
| 66944119 | 66933779 | TACCACATTCACAAGAACTGTTCTTCATAT | |
| chrX: 66763874- | chrX: 66934890- | GTATGGACAATTTGCTTGATACCTGTACAATTTAATTCTCATCCTTCCATGTGCCTTCACATTCACACATTCCACCAGAAGACCAAGGTT | 1316 |
| 66944119 | 66935009 | CACCAGCCAAAAGCTTTTCTTGCTCCCCAC | |
| chrX: 66763874- | chrX: 66935250- | ATGAGGGACTCTAGAATCCCTGATACCTGGAAGGCCTAGGATCTAAAAGAAAAGAACAGGGAAATGGGGCTATATGAGTGGACAGGGACC | 1317 |
| 66944119 | 66935369 | AACCAAGCAGAACAATGTGTCTGGATAATG | |
| chrX: 66763874- | chrX: 66934230- | AATGCTAAGCCACTGCTTTCACGAAACTCAATTTTAGCTACCACTTGCCTTGCCTAGAAGCTCATGCATGGACCCCAAGGTGAAATTGTG | 1318 |
| 66944119 | 66934349 | TTCTCTGAAGACCTCGGCTGGCAGATGTAC | |
| chrX: 66763874- | chrX: 66934290- | CTCATGCATGGACCCCAAGGTGAAATTGTGTTCTCTGAAGACCTCGGCTGGCAGATGTACTACAGCAGCAAAGATTTCCAAACTGGCCTT | 1319 |
| 66944119 | 66934409 | TCTTTGAGCCCATTCTCCCAGACTAGACAG | |
| chrX: 66763874- | chrX: 66934770- | TTGAGTGCTAAGCAGCTCTCATTAAGGACGGTTAATTAATATTATGGCCAAATTAAGCTTTCCCTTTTCTCTCCTCTTTGTTAGTTCGGT | 1320 |
| 66944119 | 66934889 | GGCATTTTAGGGAGAAAAAAATAAGCATCA | |
| chrX: 66763874- | chrX: 66935070- | GTTGCTACATGCTTAGACCCTGCTTCTTATTTCCTGCTGAGAAGGGTCAGTCCAAGGCATTCTGTGCTACAGAAGGGTTCCAAGCAGGAA | 1321 |
| 66944119 | 66935189 | CTACTCTGGGATCTGAGGCTCCAGCCGGTC | |
| chrX: 66763874- | chrX: 66934650- | GATAGTCATGTAGGCTACTTCAGAGATTGGGCATTAGAGAACAGAGTCAGGTATTATAATCAGATTAGACTCTAGGGAGGTTAGCCAGCC | 1322 |
| 66944119 | 66934769 | ATATTGCTGATATGTGCACAGTTACTGGGT | |
| chrX: 66763874- | chrX: 66934710- | CAGATTAGACTCTAGGGAGGTTAGCCAGCCATATTGCTGATATGTGCACAGTTACTGGGTTTGAGTGCTAAGCAGCTCTCATTAAGGACG | 1323 |
| 66944119 | 66934829 | GTTAATTAATATTATGGCCAAATTAAGCTT | |
| chrX: 66763874- | chrX: 66935310- | GAAATGGGGCTATATGAGTGGACAGGGACCAACCAAGCAGAACAATGTGTCTGGATAATGTAGACTTCAGACCTGATCCTATGGCTGACA | 1324 |
| 66944119 | 66935429 | AAAGCTGGTGACCTTGGTAGTTCCTGAGCT | |
| chrX: 66763874- | chrX: 66935130- | TCTGTGCTACAGAAGGGTTCCAAGCAGGAACTACTCTGGGATCTGAGGCTCCAGCCGGTCTGTCAGCGTGTCATTACAGTGAAGGTGGGA | 1325 |
| 66944119 | 66935249 | AGCACAGGCCTGGGAGCTAAGACTGCTAAG | |
| chrX: 66763874- | chrX: 66935010- | TGCCTCCTACCCAAGATATTCAGGGTCAACCTCCCAGGCCTCTTCTCTAAGAGATCCTTGGTTGCTACATGCTTAGACCCTGCTTCTTAT | 1326 |
| 66944119 | 66935129 | TTCCTGCTGAGAAGGGTCAGTCCAAGGCAT | |
| chrX: 66763874- | chrX: 66934470- | AAACAGAGTCTCAAAGAAACTGCTTCTGCTCCCTAGCGTGTTTAATGTGTTTCAGAACCTGAGAATGACTCCTCTCTGTTTCTCCAGAAC | 1327 |
| 66944119 | 66934589 | AGCCTAACACAGTGGCAAATGGGTGTTGAG | |
| chrX: 66763874- | chrX: 66934950- | ATTCACACATTCCACCAGAAGACCAAGGTTCACCAGCCAAAAGCTTTTCTTGCTCCCCACTGCCTCCTACCCAAGATATTCAGGGTCAAC | 1328 |
| 66944119 | 66935069 | CTCCCAGGCCTCTTCTCTAAGAGATCCTTG | |
| chrX: 66763874- | chrX: 66934410- | GAGACTACAAGTTTCTGCTGCACATGAAAAAAATATGATGTCAATCGGATTCTAGTGAGAAAACAGAGTCTCAAAGAAACTGCTTCTGCT | 1329 |
| 66944119 | 66934529 | CCCTAGCGTGTTTAATGTGTTTCAGAACCT | |
| chrX: 66763874- | chrX: 66935190- | TGTCAGCGTGTCATTACAGTGAAGGTGGGAAGCACAGGCCTGGGAGCTAAGACTGCTAAGATGAGGGACTCTAGAATCCCTGATACCTGG | 1330 |
| 66944119 | 66935309 | AAGGCCTAGGATCTAAAAGAAAAGAACAGG | |
| chrX: 66763874- | chrX: 66934530- | GAGAATGACTCCTCTCTGTTTCTCCAGAACAGCCTAACACAGTGGCAAATGGGTGTTGAGTGAATGCATACTTAAGGAAATCTGTAGGGT | 1331 |
| 66944119 | 66934649 | TGCAGCTACTCTTTCCTCAAGTAATCCCTT | |
| chrX: 66763874- | chrX: 66934350- | TACAGCAGCAAAGATTTCCAAACTGGCCTTTCTTTGAGCCCATTCTCCCAGACTAGACAGGAGACTACAAGTTTCTGCTGCACATGAAAA | 1332 |
| 66944119 | 66934469 | AAATATGATGTCAATCGGATTCTAGTGAGA | |
| chrX: 66763874- | chrX: 66934590- | TGAATGCATACTTAAGGAAATCTGTAGGGTTGCAGCTACTCTTTCCTCAAGTAATCCCTTGATAGTCATGTAGGCTACTTCAGAGATTGG | 1333 |
| 66944119 | 66934709 | GCATTAGAGAACAGAGTCAGGTATTATAAT | |
| chrX: 66763874- | chrX: 66934830- | TCCCTTTTCTCTCCTCTTTGTTAGTTCGGTGGCATTTTAGGGAGAAAAAAATAAGCATCAGTATGGACAATTTGCTTGATACCTGTACAA | 1334 |
| 66944119 | 66934949 | TTTAATTCTCATCCTTCCATGTGCCTTCAC | |
| chrX: 66763874- | chrX: 66935755- | GGTGATGCCTTTATAACTTTAAGCATCCAACTGTTTCAAAAACTCCAGGAGAACATGGCCATGTCTGTTCTACCTGTGTATTATTGTAGA | 1335 |
| 66944119 | 66935874 | CGTAGCTTCTGGGAGCCTCTGCTCtctgag | |
| chrX: 66763874- | chrX: 66935695- | TTTCAGATAATTCTGGATTTGTTGGTGAGAAGAGAGAGTGTTGGTAGGGACGAGCTCTGAGGTGATGCCTTTATAACTTTAAGCATCCAA | 1336 |
| 66944119 | 66935814 | CTGTTTCAAAAACTCCAGGAGAACATGGCC | |
| chrX: 66763874- | chrX: 66935635- | gaaagtgAGAAGTTTGATCTAAATTTGGGGAAGCATTCCTAATGAGGTATGATGACAAAATTTCAGATAATTCTGGATTTGTTGGTGAGA | 1337 |
| 66944119 | 66935754 | AGAGAGAGTGTTGGTAGGGACGAGCTCTGA | |
| chrX: 66763874- | chrX: 66936430- | AGGGTGGGAAATTAGTAGGGCCAGGGTACCTATTGAGTAGAAAAGAATGGAGAGGAAATGCCAGGCAGAAAGAGGATGGACGCAAGAGAG | 1338 |
| 66944119 | 66936549 | GGAACATGAAAGTGGTGAACAGGTGGCAGT | |
| chrX: 66763874- | chrX: 66936490- | CCAGGCAGAAAGAGGATGGACGCAAGAGAGGGAACATGAAAGTGGTGAACAGGTGGCAGTGGCTGTCAAGACATCTCTCCATACCCTGTA | 1339 |
| 66944119 | 66936609 | CACTGTATGTAAtatccatctcccagggtt | |
| chrX: 66763874- | chrX: 66936370- | taggcaggacataaagggcCTATTCATGTATAATGATGGCAGTAAGATGAGGATGGCAGTAGGGTGGGAAATTAGTAGGGCCAGGGTACC | 1340 |
| 66944119 | 66936489 | TATTGAGTAGAAAAGAATGGAGAGGAAATG | |
| chrX: 66763874- | chrX: 66937621- | TGCCCTGGCTTTCAGCCAACTGGCAGGAGCCCAGGAGGATGGTGCTGAGACCACCCCTTTCACACCCAAGAACCAATCCTAGTCATATTT | 1341 |
| 66944119 | 66937740 | CTGGTCTGCTTTGCAGCTTATCTCAAAACC | |
| chrX: 66763874- | chrX: 66937381- | CATGGTGTTTGCCATGGGCTGGCGATCCTTCACCAATGTCAACTCCAGGATGCTCTACTTCGCCCCTGATCTGGTTTTCAATGAGTAAGT | 1342 |
| 66944119 | 66937500 | GCTCCTGGGGCCCAGACCTCACTAAAATAC | |
| chrX: 66763874- | chrX: 66937741- | ACATGGAAAGATTCCTCCCCTTCACATATAAAAGAGGCAGAAAGACTCTGGCTTTAAGGGCTGGAGTTTCTTGGGTTCTTTTGCTACCAC | 1343 |
| 66944119 | 66937860 | CAAAGGCTACTTCTagtcaccatttgctga | |
| chrX: 66763874- | chrX: 66937561- | ATTTGATCTGCAGTTGTCGCAGCGGATGCCCCAGCCAGCCAATCCAGTATGAGGCGGCTTTGCCCTGGCTTTCAGCCAACTGGCAGGAGC | 1344 |
| 66944119 | 66937680 | CCAGGAGGATGGTGCTGAGACCACCCCTTT | |
| chrX: 66763874- | chrX: 66937141- | ttatcctcaccatatagTTTGTGCTTTTCCCCACCACCCCTTAATGGCCAGCCTGGATGGTCCCTGGGGATCCTTAGGGGATGCCCGAAT | 1345 |
| 66944119 | 66937260 | ACCAGAGCATCTCTGCCCAACAGGGACTCA | |
| chrX: 66763874- | chrX: 66937681- | CACACCCAAGAACCAATCCTAGTCATATTTCTGGTCTGCTTTGCAGCTTATCTCAAAACCACATGGAAAGATTCCTCCCCTTCACATATA | 1346 |
| 66944119 | 66937800 | AAAGAGGCAGAAAGACTCTGGCTTTAAGGG | |
| chrX: 66763874- | chrX: 66937501- | AGCAGCTTGGCCAGACCTGGTTGGTGGTGATGGTGATGGGGTGACAGTGAAGCTTAGCTCATTTGATCTGCAGTTGTCGCAGCGGATGCC | 1347 |
| 66944119 | 66937620 | CCAGCCAGCCAATCCAGTATGAGGCGGCTT | |
| chrX: 66763874- | chrX: 66937441- | CGCCCCTGATCTGGTTTTCAATGAGTAAGTGCTCCTGGGGCCCAGACCTCACTAAAATACAGCAGCTTGGCCAGACCTGGTTGGTGGTGA | 1348 |
| 66944119 | 66937560 | TGGTGATGGGGTGACAGTGAAGCTTAGCTC | |
| chrX: 66763874- | chrX: 66937321- | CTTCCGCAACTTACACGTGGACGACCAGATGGCTGTCATTCAGTACTCCTGGATGGGGCTCATGGTGTTTGCCATGGGCTGGCGATCCTT | 1349 |
| 66944119 | 66937440 | CACCAATGTCAACTCCAGGATGCTCTACTT | |
| chrX: 66763874- | chrX: 66937261- | GACTTAGCTCAACCCGTCAGTACCCAGACTGACCACTGCCTCTGCCTCTTCTTCTCCAGGCTTCCGCAACTTACACGTGGACGACCAGAT | 1350 |
| 66944119 | 66937380 | GGCTGTCATTCAGTACTCCTGGATGGGGCT | |
| chrX: 66763874- | chrX: 66937201- | TCCCTGGGGATCCTTAGGGGATGCCCGAATACCAGAGCATCTCTGCCCAACAGGGACTCAGACTTAGCTCAACCCGTCAGTACCCAGACT | 1351 |
| 66944119 | 66937320 | GACCACTGCCTCTGCCTCTTCTTCTCCAGG | |
| chrX: 66763874- | chrX: 66938098- | TGGGAAGGAGTACCTCTACCGCTCTCTGGCTGTGTGACCTTGGTACTGCCATTTTCCTTCCCTTAAACAGCTTTAATTAATACCTGCCCT | 1352 |
| 66944119 | 66938217 | GCCACCAGCTCCATATAACATCATGAATTT | |
| chrX: 66763874- | chrX: 66938927- | TGGTTCCTTCCAGTACAGCACTGCAATGCGAGGGAGAGTGAGCGTCCCCAGCTGCCCTCTCCCAACCCTGCCAGCCTGGTAGCCAAAAGC | 1353 |
| 66944119 | 66939046 | TAAGAATAACCACTAGGCTTTTGGCACAAA | |
| chrX: 66763874- | chrX: 66939587- | CAGCCCAAACTGCTCCCACACATTTCTGACACCCACTGAAGAGGCAGTACTCTCCAGTTGAGTGCAACTAATCCCTGCCAGCCTTCCTAA | 1354 |
| 66944119 | 66939706 | GGTGCTAATGGGGAGCCTCAGACCCAAAGA | |
| chrX: 66763874- | chrX: 66939407- | TCCTCCTGTAGGAAGCCACTGTGTTAGAGCTCTCAGGGTGTCTACAAACATCTAGATAAGTGTTTCTCAACATGGATTCTGTTGACATAT | 1355 |
| 66944119 | 66939526 | TGGGAAAAATAATTTTGTCATTATGTAGAA | |
| chrX: 66763874- | chrX: 66939047- | CTGCTTTGTGGTTTTCAGATCTCCGCAAAGTTGCCTATGATGCCATCTTCTGGGGCAGGCCTTGAAAAGCCCCCTAACTGTTCATCTCCC | 1356 |
| 66944119 | 66939166 | ATCCTTAAACCCCTGCTGCCCTTAAGCAGT | |
| chrX: 66763874- | chrX: 66938987- | CCCAACCCTGCCAGCCTGGTAGCCAAAAGCTAAGAATAACCACTAGGCTTTTGGCACAAACTGCTTTGTGGTTTTCAGATCTCCGCAAAG | 1357 |
| 66944119 | 66939106 | TTGCCTATGATGCCATCTTCTGGGGCAGGC | |
| chrX: 66763874- | chrX: 66939107- | CTTGAAAAGCCCCCTAACTGTTCATCTCCCATCCTTAAACCCCTGCTGCCCTTAAGCAGTTGAATCAACTCCATGAGCACCTGCTCTACC | 1358 |
| 66944119 | 66939226 | TTCCCCAGAGCCCTGAGACCTTTGGAGCTT | |
| chrX: 66763874- | chrX: 66938807- | TCCCAAGGACCTTGAGCTAGTCTCACCACAGAGAATCCTTCCAGTCAGGACAGGAATTGACCTTCCCCCCTCTTCAGCCCTCTAACCCAG | 1359 |
| 66944119 | 66938926 | AAGAGTCTTAAAATAAAATCTACAGGCCAA | |
| chrX: 66763874- | chrX: 66939647- | AGTGCAACTAATCCCTGCCAGCCTTCCTAAGGTGCTAATGGGGAGCCTCAGACCCAAAGAGAGAGAGAAGAACTTGTCCAATGTAGGTCA | 1360 |
| 66944119 | 66939766 | ACCCATTTGCTGATCTCTTCAACACCAAGC | |
| chrX: 66763874- | chrX: 66939227- | TGAAAAGTGATAATTGGTTGTTCTCTAAATCCTCATTTCCTTCTCTGCCTCTAAGTAAGCATGTGGCATCCCACCTCGGCTTCCTGGTCC | 1361 |
| 66944119 | 66939346 | AGTCTTGTTCATCTTATAAAAAGGCCTCCC | |
| chrX: 66763874- | chrX: 66938867- | CCTTCCCCCCTCTTCAGCCCTCTAACCCAGAAGAGTCTTAAAATAAAATCTACAGGCCAATGGTTCCTTCCAGTACAGCACTGCAATGCG | 1362 |
| 66944119 | 66938986 | AGGGAGAGTGAGCGTCCCCAGCTGCCCTCT | |
| chrX: 66763874- | chrX: 66939347- | TACGGGGTCAGAGGCCTAGACCCATCAAACCCAGGGCTCCTGAAACAATAGGACCCCTATTCCTCCTGTAGGAAGCCACTGTGTTAGAGC | 1363 |
| 66944119 | 66939466 | TCTCAGGGTGTCTACAAACATCTAGATAAG | |
| chrX: 66763874- | chrX: 66939707- | GAGAGAGAAGAACTTGTCCAATGTAGGTCAACCCATTTGCTGATCTCTTCAACACCAAGCTCTATTATCAGCCCTGTTTTTTTCTTTCTT | 1364 |
| 66944119 | 66939826 | TCTCTCTTTGTAGAGATCACATGTTGTGAG | |
| chrX: 66763874- | chrX: 66939287- | ATGTGGCATCCCACCTCGGCTTCCTGGTCCAGTCTTGTTCATCTTATAAAAAGGCCTCCCTACGGGGTCAGAGGCCTAGACCCATCAAAC | 1365 |
| 66944119 | 66939406 | CCAGGGCTCCTGAAACAATAGGACCCCTAT | |
| chrX: 66763874- | chrX: 66938747- | CTATGTCTGCTGTATGAGGTCCCTCGATGGCATTGTGAATGGAGCTGGCCAGAGAAATCTTCCCAAGGACCTTGAGCTAGTCTCACCACA | 1366 |
| 66944119 | 66938866 | GAGAATCCTTCCAGTCAGGACAGGAATTGA | |
| chrX: 66763874- | chrX: 66939167- | TGAATCAACTCCATGAGCACCTGCTCTACCTTCCCCAGAGCCCTGAGACCTTTGGAGCTTTGAAAAGTGATAATTGGTTGTTCTCTAAAT | 1367 |
| 66944119 | 66939286 | CCTCATTTCCTTCTCTGCCTCTAAGTAAGC | |
| chrX: 66763874- | chrX: 66939527- | TATGGTTAACATACCTGGCACCAGCCTACTCTATACCAAATAGGATTCCAGTCATTCTGACAGCCCAAACTGCTCCCACACATTTCTGAC | 1368 |
| 66944119 | 66939646 | ACCCACTGAAGAGGCAGTACTCTCCAGTTG | |
| chrX: 66763874- | chrX: 66939467- | TGTTTCTCAACATGGATTCTGTTGACATATTGGGAAAAATAATTTTGTCATTATGTAGAATATGGTTAACATACCTGGCACCAGCCTACT | 1369 |
| 66944119 | 66939586 | CTATACCAAATAGGATTCCAGTCATTCTGA | |
| chrX: 66763874- | chrX: 66940463- | CCTGAGTTGAATAATTCTACACCATCTGCCCTCTTCTCTCTTTCTCCAGGACAGCCAAGATCTCTCTGAGATAGGATGCTGAGCTTCCAC | 1370 |
| 66944119 | 66940582 | CCAGACAATACCAGGCCTGCTCATCCTATG | |
| chrX: 66763874- | chrX: 66940343- | CTCAGGGTCAGCCAATCTTACTAAAAAATTCTCTACAGTGAAAGAGCTTGGAGCAACACTGTTCTGCTCAATTGATTTGTGATACCATCT | 1371 |
| 66944119 | 66940462 | AAACACTTCCTCTTTCTAGTTGGGCTTCAG | |
| chrX: 66763874- | chrX: 66940523- | TCTCTCTGAGATAGGATGCTGAGCTTCCACCCAGACAATACCAGGCCTGCTCATCCTATGGAGTAGGCTAGTGGCTTGGAAACCAAAATG | 1372 |
| 66944119 | 66940642 | TCAAACCATAGCCTTTAGGCTCCATCTGGG | |
| chrX: 66763874- | chrX: 66940583- | GAGTAGGCTAGTGGCTTGGAAACCAAAATGTCAAACCATAGCCTTTAGGCTCCATCTGGGAGGTCTTTGTCCTCACCACTTAAGTGGGTG | 1373 |
| 66944119 | 66940702 | TCAAATTTCCTTCCCTTTCTGCACACGCTG | |
| chrX: 66763874- | chrX: 66940223- | GGCCTGCAGCAATGTTAAAGGAATCCTCATTCCAGCATTGTGATTTCAATGGTAAAAAGATTGCAGCATTGTCATCAACAGAGGTGGGAA | 1374 |
| 66944119 | 66940342 | AGTACATTGGAGACTGGAGCAGAGCCAGAC | |
| chrX: 66763874- | chrX: 66940403- | GTTCTGCTCAATTGATTTGTGATACCATCTAAACACTTCCTCTTTCTAGTTGGGCTTCAGCCTGAGTTGAATAATTCTACACCATCTGCC | 1375 |
| 66944119 | 66940522 | CTCTTCTCTCTTTCTCCAGGACAGCCAAGA | |
| chrX: 66763874- | chrX: 66940283- | TTGCAGCATTGTCATCAACAGAGGTGGGAAAGTACATTGGAGACTGGAGCAGAGCCAGACCTCAGGGTCAGCCAATCTTACTAAAAAATT | 1376 |
| 66944119 | 66940402 | CTCTACAGTGAAAGAGCTTGGAGCAACACT | |
| chrX: 66763874- | chrX: 66940811- | TAGAGGTATGCCATGGTCAGCCATGGAACCGAGAGGTTGCTCTTCCTTGAAAAGCTGGCCAAGCATTGGCCACTTCCCCATATAATTTAT | 1377 |
| 66944119 | 66940930 | AGGTGATAATGTGGTGATCTGTTCAGAAGT | |
| chrX: 66763874- | chrX: 66940751- | cGATTTTTGAAGTGCTGAAAACTGGAAGGCCTACTAGCATGAGGATGCTGTGTCTTCTCTTAGAGGTATGCCATGGTCAGCCATGGAACC | 1378 |
| 66944119 | 66940870 | GAGAGGTTGCTCTTCCTTGAAAAGCTGGCC | |
| chrX: 66763874- | chrX: 66940871- | AAGCATTGGCCACTTCCCCATATAATTTATAGGTGATAATGTGGTGATCTGTTCAGAAGTGACTATAATAAATGCAACTCACATATGTCT | 1379 |
| 66944119 | 66940990 | ACAGTTTCCAAACTGTGGTAAGGAGCAGCc | |
| chrX: 66763874- | chrX: 66941195- | CATTTGCAAATCTTGATGTCCTGATGTTAAGAGCTGACTACTGGGGCTTCTCCTAAAAATCCTTCATGTTGAGCTGCCTGGAAGGCAGGT | 1380 |
| 66944119 | 66941314 | TCTCATTCTGGCTGTAGCTGAGATGTTAGA | |
| chrX: 66763874- | chrX: 66941315- | ACTGTAGTCAGGGAGACCATGTGCCTCCCCCATTGTGTTCATTTGGTTAGGCTTTCCTGTCCCTGACTCAGAAAACAGAAGGGGCACAGA | 1381 |
| 66944119 | 66941434 | GACCTGGAAATTCCATGTGCTAACCCATAT | |
| chrX: 66763874- | chrX: 66941375- | CCCTGACTCAGAAAACAGAAGGGGCACAGAGACCTGGAAATTCCATGTGCTAACCCATATCCTGGCCAGAGAAGATGAGTAGTTATCAGG | 1382 |
| 66944119 | 66941494 | GTGTCAGGATTTTGGAAAACAGAGAGAGAa | |
| chrX: 66763874- | chrX: 66941255- | CCTTCATGTTGAGCTGCCTGGAAGGCAGGTTCTCATTCTGGCTGTAGCTGAGATGTTAGAACTGTAGTCAGGGAGACCATGTGCCTCCCC | 1383 |
| 66944119 | 66941374 | CATTGTGTTCATTTGGTTAGGCTTTCCTGT | |
| chrX: 66763874- | chrX: 66941135- | aATTTCTAGTATTCCAGGCCAGAGGCAAAGGGGTCAACAGGATGACCAAACACTTCGGGTCATTTGCAAATCTTGATGTCCTGATGTTAA | 1384 |
| 66944119 | 66941254 | GAGCTGACTACTGGGGCTTCTCCTAAAAAT | |
| chrX: 66763874- | chrX: 66941914- | CCTTCTTCATTCCCCCTCCCCATCCCCACTCTACTCTCTCTCAGCATCATTTTCCTAACAAGAAACAATTTCATGACTAGAAGCCAATTT | 1385 |
| 66944119 | 66942033 | ATTTGCTAGAAGTCAACCTCCATCAGATTC | |
| chrX: 66763874- | chrX: 66942214- | ATCTATGTAAGCAACTCAGATAGGATTTGTATGGCAGCCAAGGAACTTTTCTTTAATATCTTTTCTAAGAGCCCTCTCTTAGCCCCTACG | 1386 |
| 66944119 | 66942333 | GAGGGAGAAGGGCAAAATTTGATATTCAAA | |
| chrX: 66763874- | chrX: 66942934- | CCCTCTGGCTTTGAGTGTGGTCCAGGAAGAAAATGTGGTGAAGAAAAGAACACGGGTCACAGTGTCCCAGCTGGATATTGTGAAAGGGGT | 1387 |
| 66944119 | 66943053 | GGAGGAGTTGAGAACAGAGCAGTTGGGACT | |
| chrX: 66763874- | chrX: 66942034- | CCCACCTATCCCCAGTCTGTCTTTGGGACAAGGCCTTTTTGACTGGTTACAGCAGGTCTCTGAATTTTTCCATAGCTTCTGCTATAGAAA | 1388 |
| 66944119 | 66942153 | CAGACATGGGCCACCTTGTATTCTTTGCAG | |
| chrX: 66763874- | chrX: 66942874- | AGCCAATGATAATATGCTTCTCTAGAGTCTGGCACCACCTGTTGGGAGGTGCTTCCATTCCCCTCTGGCTTTGAGTGTGGTCCAGGAAGA | 1389 |
| 66944119 | 66942993 | AAATGTGGTGAAGAAAAGAACACGGGTCAC | |
| chrX: 66763874- | chrX: 66942514- | GGCCCCAAGCACACAGACTTCAACTAACAGGAAGCCAAGTAGATGGTTCCCTGTGGGGGTGGGGGTCAAGTCTGTGGTCAGAAAACTTGG | 1390 |
| 66944119 | 66942633 | TGCTTTGTCTAATGCTCCTTCGTGGGCATG | |
| chrX: 66763874- | chrX: 66941734- | AAGAGTTTGGATGGCTCCAAATCACCCCCCAGGAATTCCTGTGCATGAAAGCACTGCTACTCTTCAGCATTAGTAAGTGCCTAGAAGTGC | 1391 |
| 66944119 | 66941853 | AGGGAATGCCCCCTGAGGGCACAGAGATTC | |
| chrX: 66763874- | chrX: 66941674- | GGTACCGCATGCACAAGTCCCGGATGTACAGCCAGTGTGTCCGAATGAGGCACCTCTCTCAAGAGTTTGGATGGCTCCAAATCACCCCCC | 1392 |
| 66944119 | 66941793 | AGGAATTCCTGTGCATGAAAGCACTGCTAC | |
| chrX: 66763874- | chrX: 66942634- | CTTCCCCTCCCCATTCTGTCTTCATCCCACATCAGTTCCAGTGGATGGGCTGAAAAATCAAAAATTCTTTGATGAACTTCGAATGAACTA | 1393 |
| 66944119 | 66942753 | CATCAAGGAACTCGATCGTATCATTGCATG | |
| chrX: 66763874- | chrX: 66941554- | GCAGGAGAAACAGCAAGCTCTTCTTGGAAAACCTGGCGAGGGATGGCAATCAGAGACATTCCCTCTGGGCTTATTGTAAACTTCCCCTCA | 1394 |
| 66944119 | 66941673 | TTCCTTTTTCCTCTGTGTATCTCCTTCCCA | |
| chrX: 66763874- | chrX: 66942394- | CTAAAAAATGAGTATCTGAGAAGAGTAGAAAAAGAAAAGGTTCAGGAAATTTGATTTACTTGACTCCTTTCAGATCGGATCCAGCTATCC | 1395 |
| 66944119 | 66942513 | TTTCCCCTGAGATCTCCCTGACAGACTGAA | |
| chrX: 66763874- | chrX: 66941974- | AGAAACAATTTCATGACTAGAAGCCAATTTATTTGCTAGAAGTCAACCTCCATCAGATTCCCCACCTATCCCCAGTCTGTCTTTGGGACA | 1396 |
| 66944119 | 66942093 | AGGCCTTTTTGACTGGTTACAGCAGGTCTC | |
| chrX: 66763874- | chrX: 66942814- | CTCCGTGCAGCCTGTAAGCAAACGATGGAGGGTGCTTTATCAGGGAGAACAGCCTGATAGAGCCAATGATAATATGCTTCTCTAGAGTCT | 1397 |
| 66944119 | 66942933 | GGCACCACCTGTTGGGAGGTGCTTCCATTC | |
| chrX: 66763874- | chrX: 66942994- | AGTGTCCCAGCTGGATATTGTGAAAGGGGTGGAGGAGTTGAGAACAGAGCAGTTGGGACTCAGGGAAGGGACTTGCAGCAGATGAATTCT | 1398 |
| 66944119 | 66943113 | CTAGGCAGACAAAACAGACCTGGATGTTTT | |
| chrX: 66763874- | chrX: 66942274- | TTTTCTAAGAGCCCTCTCTTAGCCCCTACGGAGGGAGAAGGGCAAAATTTGATATTCAAAGCTATGTGTTTTGGTTATCTAAATCAGGGT | 1399 |
| 66944119 | 66942393 | TTTACTGTGAATGACATAAAAGCTTAGGTC | |
| chrX: 66763874- | chrX: 66942154- | GGCAGTAGAGCAGGAGGCATTTCCTCCTGGAAAGATTTCCTCTTCTGCCAACAGGAGGAGATCTATGTAAGCAACTCAGATAGGATTTGT | 1400 |
| 66944119 | 66942273 | ATGGCAGCCAAGGAACTTTTCTTTAATATC | |
| chrX: 66763874- | chrX: 66942094- | TGAATTTTTCCATAGCTTCTGCTATAGAAACAGACATGGGCCACCTTGTATTCTTTGCAGGGCAGTAGAGCAGGAGGCATTTCCTCCTGG | 1401 |
| 66944119 | 66942213 | AAAGATTTCCTCTTCTGCCAACAGGAGGAG | |
| chrX: 66763874- | chrX: 66942334- | GCTATGTGTTTTGGTTATCTAAATCAGGGTTTTACTGTGAATGACATAAAAGCTTAGGTCCTAAAAAATGAGTATCTGAGAAGAGTAGAA | 1402 |
| 66944119 | 66942453 | AAAGAAAAGGTTCAGGAAATTTGATTTACT | |
| chrX: 66763874- | chrX: 66942754- | CAAAAGAAAAAATCCCACATCCTGCTCAAGACGCTTCTACCAGCTCACCAAGCTCCTGGACTCCGTGCAGCCTGTAAGCAAACGATGGAG | 1403 |
| 66944119 | 66942873 | GGTGCTTTATCAGGGAGAACAGCCTGATAG | |
| chrX: 66763874- | chrX: 66942694- | AAAATTCTTTGATGAACTTCGAATGAACTACATCAAGGAACTCGATCGTATCATTGCATGCAAAAGAAAAAATCCCACATCCTGCTCAAG | 1404 |
| 66944119 | 66942813 | ACGCTTCTACCAGCTCACCAAGCTCCTGGA | |
| chrX: 66763874- | chrX: 66941794- | TCTTCAGCATTAGTAAGTGCCTAGAAGTGCAGGGAATGCCCCCTGAGGGCACAGAGATTCAGAGAGGACCACTTTTGCCATTAAAACATT | 1405 |
| 66944119 | 66941913 | ATTAGGGAAAAGCCAGCTCCTGGACATTTC | |
| chrX: 66763874- | chrX: 66942574- | GGGGGTCAAGTCTGTGGTCAGAAAACTTGGTGCTTTGTCTAATGCTCCTTCGTGGGCATGCTTCCCCTCCCCATTCTGTCTTCATCCCAC | 1406 |
| 66944119 | 66942693 | ATCAGTTCCAGTGGATGGGCTGAAAAATCA | |
| chrX: 66763874- | chrX: 66941854- | AGAGAGGACCACTTTTGCCATTAAAACATTATTAGGGAAAAGCCAGCTCCTGGACATTTCCCTTCTTCATTCCCCCTCCCCATCCCCACT | 1407 |
| 66944119 | 66941973 | CTACTCTCTCTCAGCATCATTTTCCTAACA | |
| chrX: 66763874- | chrX: 66941614- | CCCTCTGGGCTTATTGTAAACTTCCCCTCATTCCTTTTTCCTCTGTGTATCTCCTTCCCAGGTACCGCATGCACAAGTCCCGGATGTACA | 1408 |
| 66944119 | 66941733 | GCCAGTGTGTCCGAATGAGGCACCTCTCTC | |
| chrX: 66763874- | chrX: 66942454- | TGACTCCTTTCAGATCGGATCCAGCTATCCTTTCCCCTGAGATCTCCCTGACAGACTGAAGGCCCCAAGCACACAGACTTCAACTAACAG | 1409 |
| 66944119 | 66942573 | GAAGCCAAGTAGATGGTTCCCTGTGGGGGT | |
| chrX: 66763874- | chrX: 66943216- | gagagaTGGAGTGCGGAGGCTTGGGTGAGAGCACAAGCTGGAGAAGTCTTGAGTCAGAGAGCTTACAATGGTATAAGACATCTCTTGGGA | 1410 |
| 66944119 | 66943335 | GCCCTCAGTGACTCCATGGAGACCAtttct | |
| chrX: 66763874- | chrX: 66943442- | GACCAAAAATCAGAGGTTGGGGAAGAGGCTAGCAGAGGCCACCTCCTTGTCAACCCTGTTTTTCTCCCTCTTATTGTTCCCTACAGATTG | 1411 |
| 66944119 | 66943561 | CGAGAGAGCTGCATCAGTTCACTTTTGACC | |
| chrX: 66763874- | chrX: 66943982- | CTCATATGGCCCAGTGTCAAGTTGTGCTTGTTTACAGCACTACTCTGTGCCAGCCACACAAACGTTTACTTATCTTATGCCACGGGAAGT | 1412 |
| 66944119 | 66944101 | TTAGAGAGCTAAGATTATCTGGGGAAATCa | |
| chrX: 66763874- | chrX: 66943922- | CCATTGTGGCTCCTATCTGTGTTTTGAATGGTGTTGTATGCCTTTAAATCTGTGATGATCCTCATATGGCCCAGTGTCAAGTTGTGCTTG | 1413 |
| 66944119 | 66944041 | TTTACAGCACTACTCTGTGCCAGCCACACA | |
| chrX: 66763874- | chrX: 66943742- | CTGTTATAACTCTGCACTACTCCTCTGCAGTGCCTTGGGGAATTTCCTCTATTGATGTACAGTCTGTCATGAACATGTTCCTGAATTCTA | 1414 |
| 66944119 | 66943861 | TTTGCTGGGCTTTTTTTTTCTCTTTCTCTC | |
| chrX: 66763874- | chrX: 66943622- | CTGTGCAAGTGCCCAAGATCCTTTCTGGGAAAGTCAAGCCCATCTATTTCCACACCCAGTGAAGCATTGGAAACCCTATTTCCCCACCCC | 1415 |
| 66944119 | 66943741 | AGCTCATGCCCCCTTTCAGATGTCTTCTGC | |
| chrX: 66763874- | chrX: 66943682- | GAAGCATTGGAAACCCTATTTCCCCACCCCAGCTCATGCCCCCTTTCAGATGTCTTCTGCCTGTTATAACTCTGCACTACTCCTCTGCAG | 1416 |
| 66944119 | 66943801 | TGCCTTGGGGAATTTCCTCTATTGATGTAC | |
| chrX: 66763874- | chrX: 66943862- | CTTTCTTTTTCTTCTTCCCTCCCTATCTAACCCTCCCATGGCACCTTCAGACTTTGCTTCCCATTGTGGCTCCTATCTGTGTTTTGAATG | 1417 |
| 66944119 | 66943981 | GTGTTGTATGCCTTTAAATCTGTGATGATC | |
| chrX: 66763874- | chrX: 66943802- | AGTCTGTCATGAACATGTTCCTGAATTCTATTTGCTGGGCTTTTTTTTTCTCTTTCTCTCCTTTCTTTTTCTTCTTCCCTCCCTATCTAA | 1418 |
| 66944119 | 66943921 | CCCTCCCATGGCACCTTCAGACTTTGCTTC | |
| chrX: 66763874- | chrX: 66943382- | cGACCTCATGGGGGAGGACCAAGGAAGTACGGGGAAGGGGGAGGAAACAAAAGGCTGAAAGACCAAAAATCAGAGGTTGGGGAAGAGGCT | 1419 |
| 66944119 | 66943501 | AGCAGAGGCCACCTCCTTGTCAACCCTGTT | |
| chrX: 66763874- | chrX: 66943502- | TTTCTCCCTCTTATTGTTCCCTACAGATTGCGAGAGAGCTGCATCAGTTCACTTTTGACCTGCTAATCAAGTCACACATGGTGAGCGTGG | 1420 |
| 66944119 | 66943621 | ACTTTCCGGAAATGATGGCAGAGATCATCT | |
| chrX: 66763874- | chrX: 66943562- | TGCTAATCAAGTCACACATGGTGAGCGTGGACTTTCCGGAAATGATGGCAGAGATCATCTCTGTGCAAGTGCCCAAGATCCTTTCTGGGA | 1421 |
| 66944119 | 66943681 | AAGTCAAGCCCATCTATTTCCACACCCAGT | |

### Immunostaining

CWR-R1 cells were grown on glass cover slips 48 hours in RPMI+10% CSS and fixed at −10° C. in acetone. Fixed cells were blocked in horse serum (Vector Laboratories, Inc., Burlingame, Calif.), incubated with primary antibodies for 30 minutes (AR N20: 1:400, AR C19, 1:100, AR V7 3 ug/mL), and washed prior to incubation with biotin-conjugated universal secondary antibody (Vector Laboratories, Inc., Burlingame, Calif.) diluted 1:4000. Immunoreactive cells were visualized by incubation in peroxidase substrate (Sigma-Aldrich, St. Louis, Mo.).

### Analysis of Paired-End Sequence Data

Raw sequence data from GAIIx sequencing was de-multiplexed and filtered using Illumina pass/fail (P/F) information to remove poor-quality reads. Raw sequences were converted to FASTQ format and read quality was verified using fastQC (Babraham Institute, Cambridge, United Kingdom). Filtered reads were mapped to the hg19 build of the reference genome using the Burrows-Wheeler Alignment tool (BWA) (Li and Durbin, 2009 Bioinformatics 25:1754-60) and Novoalign version 2.07.05 (Novocraft, Technologies Sdn Bhd, Selangor, Malaysia) with parameter settings as outlined in the Hydra workflow (Quinlan et al., 2010 Genome Res 20:623-35). Briefly, in BWA alignment seed size was 20 (“−l 20”). Up to two differences within the seed (“−k 2”), up to eight differences in each read end (“−n 8”), up to 3 gaps opened in the alignment (“−o 3”), and up to three gap extensions (“−e 3”) were allowed. BWA was then forced to search for suboptimal alignments (“−R”). In the alignment pairing phase, the maximal expected insert size (“−a”) was set to be equivalent to the median fragment size plus 10 times the median absolute deviation of the DNA fragment library. Up to 10 million possible mapping locations (“−o 10000000”) were allowed. Discordantly-mapped read pairs or read pairs that could not be aligned with BWA were collected and re-aligned using Novoalign with the following parameters: word size of 14, step size of 1, −g 0, −x 30, −r Ex 1100, −t 90, −e 5000000. All remaining discordantly-mapped read pairs or read pairs which could not be aligned with both BWA and Novoalign were used to identify structural variations using Hydra with parameters: −mld 500 −mno 1500. Output data in BAM format were visualized using Integrative Genomics Viewer 2.0. To infer SNPs/indel events from paired-end sequence data, VarScan (Koboldt et al., 2009 Bioinformatics 25:2283-5) was used with SAMtools pileup (Li et al., 2009 Bioinformatics 25:2078-9) as input and parameters (p value<0.01, min var freq>0.2, min avg qual>15, min reads2>2, min coverage>8).

### Example 2

### Cell Culture

The 22Rv1 (#CRL-2505) and LNCaP (#CRL-1740) cell lines were obtained from ATCC and cultured according to ATCC protocol. ATCC ensures authenticity of these human cell lines using short tandem repeat (STR) analyses. All experiments with these cells were performed within four months of resuscitation of frozen cell stocks prepared within three passages of receipt from ATCC. CWR-R1 cells (Gregory et al., 2001 Cancer Res 61:2892-8) were cultured in RPMI 1640+10% FBS. Authentication of the CWR-R1 cell line was performed by sequence-based validation of two signature AR gene alterations: AR H874Y point mutation and 50 kb intragenic deletion within AR intron 1 (Li et al., 2012 Oncogene 31:4759-4767). Sequence-based authentication of CWR-R1 was performed every 5-10 passages, and cells were kept in culture no longer than three months after authentication unless otherwise indicated.

For androgen response experiments, cells were cultured in RPMI 1640+10% steroid-depleted, charcoal stripped serum (CSS) for 48 hours, treated at t=0 with combinations of 1 nM DHT (Sigma-Aldrich, St. Louis, Mo.), 10 μM bicalutamide (AstraZeneca Pharmaceuticals LP, Wilmington, Del.), 1 μM enzalutamide/MDV3100 (Medivation, Inc., San Francisco, Calif.), or vehicle control (EtOH), and then harvested at indicated time points. For long-term culture experiments, CWR-R1 cells were cultured in RPMI 1640+10% CSS. Cells were trypsinized and re-seeded in the appropriate medium when flasks attained 80% confluence. For single cell cloning experiments, cells were seeded at limiting dilution in 96-well plates and wells with single cell clones were expanded.

### Transient Transfections

Cells were electroporated with siRNAs targeted to AR exon 7 (Dehm et al., 2008 Cancer Res 68:5469-77), AR exon 1 (Dehm et al., 2008 Cancer Res 68:5469-77), AR exon 2b (Dehm et al., 2008 Cancer Res 68:5469-77), AR exon CE3 (Hu et al., 2009 Cancer Res 69:16-22), or an MMTV-LUC reporter as described (Dehm et al., 2008 Cancer Res 68:5469-77). Growth of electroporated cells was monitored by crystal violet staining as described (Li et al., 2011 Cancer Res 71:2108-17). Luciferase activity was measured as described (Dehm et al., 2008 Cancer Res 68:5469-77).

### Lentiviral Infections

LNCaP cells were infected with increasing titers of lentivirus encoding AR 1/2/3/CE3 and AR Δ5/6/7 as described (Chan et al., 2012 J Biol Chem 287:19736-49). Infected cells were maintained in RPMI 1640+10% CSS for 48 h and then switched to serum free medium for 24 hours prior to lysis.

### Western Blot

Western blotting with AR NTD (Santa Cruz N-20, Santa Cruz Biotechnology, Inc., Dallas, Tex.), AR CTD (Santa Cruz C-19, Santa Cruz Biotechnology, Inc., Dallas, Tex.), and ERK-2 (Santa Cruz D-2, Santa Cruz Biotechnology, Inc., Dallas, Tex.) antibodies was performed as described (Li et al., 2011 Cancer Res 71:2108-17).

### Quantitative RT-PCR

Total RNA was extracted from 22Rv1, CWR-R1, and LNCaP cells as described (Chomczynski et al., 1987. Anal Biochem 162:156-9). Primers and quantitative reverse transcription PCR (qRT-PCR) conditions for assessment of PSA, hK2, and TMPRSS2 mRNA expression have been described (Dehm et al., 2008 Cancer Res 68:5469-77). Androgen responses and AR variant responses of M-phase specific genes were assessed using the following specific primers: CDCA5 (5′-CGTAAGAAGAAGAAAATGCCAGA; SEQ ID NO:1422 and 5′-TCAAACTCGGCATTCATGG; SEQ ID NO:1423), ZWINT (5′-GTGGGAAGGCAGCTGAAC; SEQ ID NO:1424 and 5′-CACCTCAGCCAGGACCTC; SEQ ID NO:1425), CCNA2 (5′-GGTACTGAAGTCCGGGAACC; SEQ ID NO:1426 and 5′-GAAGATCCTTAAGGGGTGCAA; SEQ ID NO:1427), and UBE2C (5′-TGGTCTGCCCTGTATGATGT; SEQ ID NO:1428 and 5′-AAAAGCTGTGGGGTTTTTCC; SEQ ID NO:1429). Akt mRNA expression levels were assessed using 5′-TCTATGGCGCTGAGATTGTG; SEQ ID NO:1430 and 5′-CTTAATGTGCCCGTCCTTGT; SEQ ID NO:1431. Fold change in mRNA expression levels were determined by the comparative Ct method using the equation 2^{-ΔΔCt}. GAPDH as calibrator as described (Dehm et al., 2008 Cancer Res 68:5469-77).

### Genomic PCR

Primers and PCR conditions for deletion-spanning PCR of the AR intron 1 deletion in CWR-R1 cells have been described (Li et al., 2012 Oncogene 31:4759-4767).

Gene Expression Analysis with Illumina Beadchips

CWR-R1 cells that had been maintained in long term culture in RPMI 1640+10% CSS were used for global gene expression profiling. These CWR-R1 cells were electroporated with siRNAs targeting AR exon 1, 7, or CE3, and seeded in RPMI+10% CSS. Following 48 hours recovery, cells were switched to serum-free RPMI 1640 and treated for 24 hours with 1 nM DHT or 0.01% (v/v) vehicle control (ethanol).

### Multiplex Ligation-Dependent Probe Assay

Multiplex ligation-dependent probe assay with custom-designed probes for the AR locus has been described (Li et al., 2012 Oncogene 31:4759-4767). The percentage of deletion-positive CWR-R1 cells was inferred by dividing copy number derived from AR locus probes targeted within the deletion by copy number derived from AR locus probes targeted outside of the deletion, and subtracting from 100%.

### Statistical Analysis

Two-tailed paired t-tests were used to assess statistical significances in promoter-reporter assays quantitative RT-PCR experiments, and cell growth experiments.

Gene Expression Analysis with Illumina Beadchips

RNA was isolated (Chomczynski et al., 1987 Anal Biochem 162:156-9), further purified using Qiagen RNeasy columns (Qiagen Inc. USA, Valencia, Calif.) as per the manufacturer's protocol, and 2 μg was submitted to the University of Minnesota's BioMedical Genomics Center for Illumina Direct Hybridization array analysis (Illumina, Inc., San Diego, Calif.). RNA quality control was performed using a NanoDrop 8000 spectrophotometer (Thermo Fisher Scientific, Waltham, Mass.) and Caliper LabChip® GX (PerkinElmer, Inc., Waltham, Mass.). Total RNA was converted to amplified biotinylated, antisense cRNA using the Illumina TotalPrep-96 RNA Amplification Kit (Life Technologies, Carlsbad, Calif.), and 150 ng of biotin-labeled cRNA was hybridized onto Illumina HumanHT-12 v4 Expression Beadchips (Illumina, Inc., San Diego, Calif.) using the HumanHT-12 v4 Expression BeadChip Kit (Illumina, Inc., San Diego, Calif.). Hybridized Beadchips were scanned with an Illumina iScan. Raw intensity data was extracted from iScan scan image files (Illumina, Inc., San Diego, Calif.) using GenomeStudio software (Illumina, Inc., San Diego, Calif.), log₂ transformed, and then imported to Partek Genomics Suite 6.6 (Partek Inc., St. Louis, Mo.). Using Partek GS, raw intensity data was quantile normalized and differential gene expression was determined by one-way analysis of variance (ANOVA) using default settings within the Gene Expression Workflow. Genes demonstrating at least 1.2-fold change in response to treatment (siAR-exon 7 vs. siAR-exon 1 or siAR-exon CE3 vs. siAR-exon CE3+1 nM DHT) with a P-value less than 0.05 were deemed differentially expressed. Heat maps were generated by unsupervised hierarchical clustering of differentially-expressed genes. Rows were scaled to mean zero and standard deviation equal to one.

### Gene Set Enrichment Analysis (GSEA)

Gene sets that have been shown to discriminate between AR variant-driven transcription and androgen/AR-driven transcription have been described (Hu et al., 2012 Cancer Res 72:3457-62). These gene sets were tested for enrichment in gene expression data from CWR-R1 cells using GSEA v2.07 (Broad Institute, Cambridge, Mass.; Subramanian et al., 2005 Proc Natl Acad Sci USA 102:15545-50). Normalized gene expression data was ranked using the Signal2Noise metric and GSEA was performed against 1000 random gene set permutations. Using the same approach, these gene sets were tested for enrichment in gene expression data from biological triplicates of LNCaP cells cultured 18 hours in 1 nM DHT vs. ethanol vehicle control (NCBI gene expression omnibus GEO dataset GSE26483) and gene expression data from biological triplicates of LNCaP cells cultured 16 hours in 100 nM DHT vs. ethanol vehicle control (GSE7868).

### Ingenuity Pathways Analysis

Differentially-expressed gene lists (Table 4) from siAR-exon 7 vs. siAR-exon 1 or siAR-exon CE3 vs. siAR-exon CE3+1 nM DHT datasets were used as input for network analysis using Ingenuity Systems Pathways Analysis (Ingenuity Systems, Redwood Calif.), a commercial application that infers the relationship between the gene set and known pathways. Default settings were used where “Ingenuity Knowledge Base (Genes Only)” was the reference set, and both direct and indirect relationships were considered to identify sub-networks enriched with the selected genes.

TABLE 4
Androgen/AR and AR-V gene lists.
Genes regulated by androgens in CWR-R1 cells transfected
with siRNA targeting the AR 1/2/3/CE3 variant

| Probeset | p-value (siCE3 + DHT | Fold-Change (siCE3 + DHT |
|---|---|---|
| ID | vs. siCE3) | vs. siCE3) |
| FKBP5 | 0.000000765 | 3.42041 |
| HES6 | 0.0000252 | 2.99365 |
| NPTX2 | 0.0000290 | 2.70784 |
| TIPARP | 0.00018481 | 2.56108 |
| PGC | 0.0001227 | 2.11727 |
| DPYSL4 | 0.00322307 | 2.07201 |
| S100P | 0.00918238 | 2.01378 |
| FOSB | 0.00925952 | 2.01271 |
| APOD | 0.00365163 | 1.96194 |
| NT5DC3 | 0.000131418 | 1.92481 |
| BHLHB2 | 0.0000462 | 1.88629 |
| LOC93622 | 0.00000105 | 1.83224 |
| DBI | 0.0495379 | 1.823 |
| SLC2A3 | 0.000000429 | 1.82085 |
| C20ORF134 | 0.01832 | 1.76719 |
| LRRC8A | 0.00154371 | 1.73076 |
| KISS1R | 0.000203808 | 1.72037 |
| ETNK2 | 0.000957634 | 1.66748 |
| MORF4L2 | 0.0327137 | 1.66342 |
| LONRF1 | 0.000244069 | 1.65684 |
| PFKFB4 | 0.000717931 | 1.64165 |
| LOX | 0.0000443 | 1.63577 |
| C13ORF15 | 0.0000716 | 1.62289 |
| MAP7D1 | 0.000298384 | 1.61134 |
| MAFB | 0.0000184 | 1.57465 |
| KCNG1 | 0.000581214 | 1.57429 |
| GPT2 | 0.000752196 | 1.5741 |
| LOC729768 | 0.00143944 | 1.57 |
| CDKN2D | 0.000747701 | 1.56915 |
| ODC1 | 0.0111117 | 1.54288 |
| GINS3 | 0.0028224 | 1.54274 |
| ATAD2 | 0.0101718 | 1.53811 |
| NCAPD3 | 0.00255412 | 1.53492 |
| GADD45G | 0.000150009 | 1.53155 |
| PDXP | 0.00713692 | 1.52905 |
| RPRC1 | 0.00202149 | 1.52871 |
| NPAS1 | 0.00781667 | 1.52563 |
| HS.10862 | 0.00578384 | 1.52122 |
| ARID3A | 0.0162702 | 1.52111 |
| COASY | 0.0354129 | 1.51137 |
| ST6GALNAC1 | 0.0020405 | 1.50806 |
| PECI | 0.00331135 | 1.50739 |
| GALNTL4 | 0.0017509 | 1.50257 |
| MRFAP1 | 0.00810637 | 1.50138 |
| SLC6A3 | 0.0000807 | 1.49554 |
| RHOB | 0.00835951 | 1.49258 |
| VPS26B | 0.00130206 | 1.49067 |
| STK39 | 0.00433322 | 1.48588 |
| SLC16A3 | 0.00929776 | 1.48435 |
| CHD5 | 0.0299714 | 1.48025 |
| CYB5A | 0.00679253 | 1.46991 |
| FOXD1 | 0.00242873 | 1.46303 |
| SCAP | 0.0126428 | 1.46236 |
| IL12A | 0.000394316 | 1.46104 |
| KLK4 | 0.0000249 | 1.45944 |
| SMAP2 | 0.000746684 | 1.45677 |
| FZD9 | 0.0146235 | 1.45435 |
| SLC6A8 | 0.0165823 | 1.45123 |
| ACSL3 | 0.0305889 | 1.44741 |
| CHPT1 | 0.0304862 | 1.43757 |
| GHR | 0.000307225 | 1.43496 |
| KLF9 | 0.0306443 | 1.43441 |
| ELOVL6 | 0.0263144 | 1.43004 |
| TSPAN33 | 0.000443836 | 1.42394 |
| DAGLB | 0.0263395 | 1.42148 |
| SPRY1 | 0.000149564 | 1.42077 |
| DDX41 | 0.0235745 | 1.41958 |
| RNF103 | 0.0412361 | 1.41885 |
| MFSD2 | 0.000233103 | 1.41715 |
| PDIA5 | 0.00236514 | 1.41424 |
| SLC2A1 | 0.0304804 | 1.41341 |
| GAL | 0.00265908 | 1.41179 |
| HSD17B1 | 0.00752451 | 1.41049 |
| ATP1A1 | 0.00405451 | 1.40997 |
| HS.567759 | 0.0452213 | 1.40225 |
| ALDOC | 0.00944538 | 1.40137 |
| HOMER2 | 0.000884402 | 1.40107 |
| CYB5D1 | 0.0244382 | 1.39359 |
| GBE1 | 0.0223831 | 1.39322 |
| XPR1 | 0.00562923 | 1.3892 |
| TMEM145 | 0.0130141 | 1.38916 |
| DDIT4 | 0.00429053 | 1.38745 |
| CRELD2 | 0.000842371 | 1.38709 |
| NCLN | 0.0480052 | 1.38528 |
| FAM174B | 0.00180786 | 1.38416 |
| BNIP3L | 0.0184164 | 1.38367 |
| CAV1 | 0.000274171 | 1.38226 |
| INSIG1 | 0.0114145 | 1.382 |
| UNC119 | 0.00791392 | 1.38082 |
| ABCC4 | 0.0000479 | 1.37975 |
| ACSS2 | 0.0441101 | 1.37795 |
| WDR41 | 0.0223552 | 1.37602 |
| ELAVL1 | 0.0134147 | 1.37425 |
| ERN1 | 0.0137994 | 1.37143 |
| LIX1L | 0.00619965 | 1.37118 |
| SLC6A10P | 0.0447379 | 1.37055 |
| GADD45B | 0.0000107 | 1.36686 |
| PFKP | 0.0308204 | 1.36356 |
| SDF2L1 | 0.02826 | 1.36025 |
| ARMET | 0.00000446 | 1.35866 |
| ZDHHC9 | 0.000411997 | 1.35843 |
| DHX32 | 0.0189874 | 1.35472 |
| ASB9 | 0.000197648 | 1.35389 |
| RHOV | 0.0480207 | 1.35327 |
| ANKZF1 | 0.00137304 | 1.35238 |
| SRPK1 | 0.0439745 | 1.34816 |
| LOC100131330 | 0.00111248 | 1.34319 |
| UBE2M | 0.0200859 | 1.34316 |
| LOC731835 | 0.00679101 | 1.34129 |
| ELF3 | 0.0027502 | 1.33743 |
| KLF15 | 0.00966167 | 1.33468 |
| C2ORF7 | 0.00132952 | 1.33407 |
| CMAS | 0.0155794 | 1.33363 |
| LOC387763 | 0.000998454 | 1.33253 |
| LOC732007 | 0.00424007 | 1.33005 |
| LASS6 | 0.0166391 | 1.32849 |
| SOX9 | 0.00292801 | 1.325 |
| PIM2 | 0.0331199 | 1.32444 |
| SERTAD2 | 0.0105981 | 1.32058 |
| F2RL1 | 0.0148943 | 1.32017 |
| EDEM1 | 0.0246777 | 1.31979 |
| BCKDK | 0.014412 | 1.31921 |
| CCRN4L | 0.0243877 | 1.31879 |
| LOC731049 | 0.0372518 | 1.31833 |
| NSDHL | 0.000336824 | 1.31779 |
| PDCD4 | 0.000390244 | 1.31662 |
| LOC154761 | 0.00084718 | 1.31563 |
| ACOT7 | 0.0187112 | 1.31464 |
| GRPEL1 | 0.00112379 | 1.31351 |
| WDR45L | 0.000258628 | 1.31336 |
| MAP6D1 | 0.00286747 | 1.31326 |
| RHOBTB2 | 0.0103439 | 1.31269 |
| GFOD2 | 0.0201284 | 1.31073 |
| HES7 | 0.0189747 | 1.30826 |
| LOC387703 | 0.0477945 | 1.30606 |
| TFB1M | 0.0333406 | 1.30595 |
| C6ORF81 | 0.000573289 | 1.30507 |
| SLC25A22 | 0.00728908 | 1.30441 |
| PCYT2 | 0.0175054 | 1.30247 |
| LOC100129668 | 0.0358644 | 1.30192 |
| PPP2CB | 0.018807 | 1.30118 |
| RNF150 | 0.0000733 | 1.30026 |
| C20ORF20 | 0.00789422 | 1.29747 |
| TPI1 | 0.0310653 | 1.29715 |
| SRM | 0.0212644 | 1.29606 |
| UPP1 | 0.000728626 | 1.29575 |
| CBLL1 | 0.000267573 | 1.29469 |
| ADRM1 | 0.00998897 | 1.29419 |
| HK2 | 0.0468556 | 1.29385 |
| ANGPTL4 | 0.0213257 | 1.29367 |
| TRIP13 | 0.0213194 | 1.29342 |
| RHOBTB3 | 0.0060568 | 1.29275 |
| COPS8 | 0.0086999 | 1.29176 |
| DLX1 | 0.00109045 | 1.29069 |
| SLC2A6 | 0.0206572 | 1.29014 |
| AGTRAP | 0.0340457 | 1.28922 |
| PEG10 | 0.00935376 | 1.28857 |
| C15ORF23 | 0.0067309 | 1.28839 |
| CCND3 | 0.00276062 | 1.28646 |
| TTF2 | 0.0323074 | 1.28254 |
| ZFP36 | 0.00731884 | 1.28192 |
| CMTM7 | 0.0155853 | 1.28075 |
| TMEM149 | 0.00838254 | 1.28011 |
| ITPKA | 0.0270287 | 1.27838 |
| WISP2 | 0.0190643 | 1.27537 |
| INSIG2 | 0.0137872 | 1.27382 |
| ITGAV | 0.0225311 | 1.27368 |
| TMEM97 | 0.000320952 | 1.27334 |
| SNCB | 0.00669929 | 1.2732 |
| CEP55 | 0.0307413 | 1.27298 |
| TOMM40 | 0.028895 | 1.27111 |
| P4HA2 | 0.00400386 | 1.26931 |
| TYMS | 0.00971626 | 1.26843 |
| SLC7A5 | 0.0381485 | 1.26765 |
| LOC286016 | 0.00811593 | 1.26733 |
| CLPTM1L | 0.0474098 | 1.26702 |
| QSOX2 | 0.0282193 | 1.26688 |
| CLK3 | 0.0060221 | 1.26677 |
| PSMD8 | 0.0267623 | 1.26564 |
| PPME1 | 0.0366065 | 1.26517 |
| TBC1D4 | 0.000291136 | 1.26468 |
| NDRG4 | 0.024933 | 1.26357 |
| JPH1 | 0.0219463 | 1.26352 |
| SHPK | 0.0234513 | 1.26349 |
| SLC25A4 | 0.00183809 | 1.26329 |
| VCP | 0.000556431 | 1.26304 |
| HK1 | 0.0110306 | 1.263 |
| IRS2 | 0.00396626 | 1.26135 |
| AP4E1 | 0.00191454 | 1.26112 |
| FAM104A | 0.032241 | 1.26104 |
| BCL2L12 | 0.0133864 | 1.26083 |
| XPNPEP1 | 0.0302627 | 1.26063 |
| MNX1 | 0.0408555 | 1.26059 |
| KCTD13 | 0.0481757 | 1.26022 |
| GMPPB | 0.0407027 | 1.25981 |
| MCCC1 | 0.0420134 | 1.2594 |
| VKORC1 | 0.00928546 | 1.258 |
| LOC440043 | 0.044316 | 1.25788 |
| PELO | 0.0014877 | 1.25784 |
| TLCD1 | 0.0326589 | 1.25627 |
| STC2 | 0.0158113 | 1.25622 |
| BARD1 | 0.00241717 | 1.25575 |
| KLHL29 | 0.0126011 | 1.25542 |
| RNF126 | 0.028332 | 1.25529 |
| NOLC1 | 0.00168326 | 1.25515 |
| EGLN1 | 0.0000882 | 1.25477 |
| EFCAB3 | 0.0102557 | 1.25391 |
| TSPYL2 | 0.0000762 | 1.25245 |
| GSTO1 | 0.000430122 | 1.25184 |
| C18ORF8 | 0.000254634 | 1.25078 |
| CAP2 | 0.00132553 | 1.2494 |
| C10ORF39 | 0.00625523 | 1.24927 |
| VCL | 0.0209055 | 1.24824 |
| PREP | 0.029758 | 1.24701 |
| PAXIP1 | 0.0204828 | 1.24655 |
| P4HA1 | 0.0197909 | 1.24645 |
| C9ORF91 | 0.0334236 | 1.24529 |
| NELL2 | 0.00945158 | 1.24498 |
| PPP2R5B | 0.0285319 | 1.24472 |
| UTP11L | 0.0138101 | 1.24439 |
| UNC5A | 0.028661 | 1.24362 |
| WDYHV1 | 0.0187308 | 1.24265 |
| INPP1 | 0.00209825 | 1.24217 |
| C16ORF57 | 0.029382 | 1.23984 |
| CNTFR | 0.0294294 | 1.23965 |
| SLC39A3 | 0.0251775 | 1.23922 |
| FAM158A | 0.0332454 | 1.23845 |
| NDUFS7 | 0.0231211 | 1.23706 |
| CYGB | 0.026593 | 1.23702 |
| DNM1L | 0.000123811 | 1.23661 |
| THBS1 | 0.00835903 | 1.23649 |
| LETM1 | 0.0330421 | 1.23633 |
| UCK2 | 0.00515094 | 1.23479 |
| CDK6 | 0.0462175 | 1.23476 |
| PSMB2 | 0.00349688 | 1.23346 |
| HAGH | 0.0224527 | 1.23204 |
| CHSY1 | 0.0236556 | 1.23094 |
| AURKB | 0.0189457 | 1.22806 |
| GSTM1 | 0.0287644 | 1.22758 |
| LYN | 0.0222753 | 1.22717 |
| TNFAIP3 | 0.00308184 | 1.22633 |
| RRP7A | 0.0353869 | 1.22592 |
| WIPI1 | 0.0134224 | 1.22561 |
| SLC25A13 | 0.0177424 | 1.22536 |
| MRPL17 | 0.021549 | 1.22493 |
| GCLM | 0.00515623 | 1.2244 |
| E2F4 | 0.0390496 | 1.2239 |
| PSMB3 | 0.00795024 | 1.22378 |
| PGAM4 | 0.0230494 | 1.2236 |
| LOC284422 | 0.0423407 | 1.2234 |
| POLS | 0.047283 | 1.22291 |
| POP1 | 0.0362152 | 1.22285 |
| KIAA0020 | 0.0184969 | 1.22209 |
| HS.19339 | 0.000981667 | 1.22152 |
| HPGD | 0.00120535 | 1.22041 |
| ALDH18A1 | 0.0129835 | 1.21894 |
| CD19 | 0.0459728 | 1.2186 |
| PDK3 | 0.00274281 | 1.21786 |
| PEBP4 | 0.0186921 | 1.21734 |
| RELL2 | 0.0375089 | 1.21723 |
| CDC45L | 0.0210284 | 1.21633 |
| CLEC16A | 0.00403489 | 1.21468 |
| C1ORF116 | 0.0346163 | 1.21467 |
| LOC100130511 | 0.00737261 | 1.21411 |
| HMOX1 | 0.0436251 | 1.21401 |
| PAICS | 0.0464648 | 1.2138 |
| NR5A2 | 0.0108161 | 1.21089 |
| FAM40A | 0.00503295 | 1.20937 |
| KIAA2010 | 0.0240744 | 1.20792 |
| KCTD9 | 0.0405817 | 1.20722 |
| WASF3 | 0.0386334 | 1.2069 |
| TMEM64 | 0.0341541 | 1.20608 |
| LOC400013 | 0.0276251 | 1.20528 |
| AXUD1 | 0.0394881 | 1.20465 |
| C16ORF93 | 0.0491096 | 1.20442 |
| IQCB1 | 0.00149515 | 1.20432 |
| HERC3 | 0.00811273 | 1.2043 |
| PIP4K2A | 0.0115962 | 1.20383 |
| CP110 | 0.00723839 | 1.20246 |
| B4GALNT1 | 0.0157536 | 1.20228 |
| DEPDC6 | 0.000487814 | 1.20222 |
| PIAS1 | 0.00155044 | 1.20128 |
| LOC728139 | 0.00626663 | −1.20083 |
| FBXW4 | 0.0246045 | −1.20091 |
| SH3BP4 | 0.0126317 | −1.20114 |
| ZNRD1 | 0.0223357 | −1.20168 |
| EPN3 | 0.0130161 | −1.20199 |
| MMP28 | 0.0011828 | −1.20202 |
| SARS | 0.00733343 | −1.20245 |
| ANTXR2 | 0.00376662 | −1.20291 |
| HSPBL2 | 0.02782 | −1.20297 |
| RND2 | 0.00720192 | −1.20454 |
| MRPS18B | 0.0369114 | −1.20559 |
| C6ORF48 | 0.0025436 | −1.2072 |
| FLJ40125 | 0.00536699 | −1.20926 |
| LOC100133008 | 0.038542 | −1.20991 |
| HS.552826 | 0.00253149 | −1.21011 |
| GOLSYN | 0.0123987 | −1.21046 |
| E2F5 | 0.0160119 | −1.21052 |
| LOC728755 | 0.0000964 | −1.2118 |
| SFTPD | 0.00208887 | −1.21269 |
| LOC100128353 | 0.0344332 | −1.21289 |
| FLOT1 | 0.0358949 | −1.2133 |
| MYD88 | 0.00630122 | −1.21387 |
| ABR | 0.00257794 | −1.21389 |
| TMEM18 | 0.000103651 | −1.21402 |
| TSPAN10 | 0.00324855 | −1.21414 |
| CGN | 0.0164543 | −1.21499 |
| APEX1 | 0.00746476 | −1.21501 |
| HNRNPA0 | 0.0389217 | −1.2153 |
| ICA1 | 0.000732285 | −1.21597 |
| LOC728229 | 0.00446282 | −1.21619 |
| PLLP | 0.000560978 | −1.2164 |
| VWF | 0.018074 | −1.2164 |
| ZSCAN18 | 0.00201812 | −1.21708 |
| GATS | 0.000890899 | −1.21746 |
| SGSM3 | 0.0442699 | −1.21784 |
| AIG1 | 0.0320108 | −1.21815 |
| PHLDA3 | 0.028983 | −1.21824 |
| HS.8038 | 0.00109786 | −1.21864 |
| TSTD1 | 0.00439713 | −1.21888 |
| RNF130 | 0.00510613 | −1.21921 |
| HS.31532 | 0.00217397 | −1.22006 |
| FLJ90086 | 0.000411319 | −1.22037 |
| ANXA11 | 0.00398072 | −1.22041 |
| LARP6 | 0.0000549 | −1.22162 |
| CYTH2 | 0.047715 | −1.22193 |
| LOC389672 | 0.0284785 | −1.22243 |
| TRIP6 | 0.0223495 | −1.22274 |
| CYB5D2 | 0.0178983 | −1.22313 |
| ARSD | 0.000477149 | −1.2234 |
| AMHR2 | 0.00640132 | −1.22391 |
| P2RX4 | 0.0195831 | −1.22396 |
| CASQ1 | 0.000382496 | −1.22455 |
| CD63 | 0.0161774 | −1.22466 |
| NCALD | 0.00744381 | −1.22516 |
| OSBPL5 | 0.0356104 | −1.22517 |
| ITGB5 | 0.0172426 | −1.2256 |
| TSPAN15 | 0.0184015 | −1.22564 |
| ACP2 | 0.0418201 | −1.2259 |
| PRPSAP1 | 0.00426606 | −1.22616 |
| IRF2BP2 | 0.00708665 | −1.22656 |
| KIAA0141 | 0.0363736 | −1.22745 |
| LPAR5 | 0.0189713 | −1.22752 |
| C9ORF103 | 0.0223365 | −1.22801 |
| GLIPR2 | 0.00215895 | −1.22817 |
| LASP1 | 0.0383098 | −1.22872 |
| PRRG2 | 0.01058 | −1.22873 |
| UNC119B | 0.0197355 | −1.22911 |
| HS.559604 | 0.0391854 | −1.22922 |
| TMEM62 | 0.000198644 | −1.22948 |
| FLJ32252 | 0.00238204 | −1.22974 |
| BCAR3 | 0.00370696 | −1.23004 |
| LOC729926 | 0.0213471 | −1.23031 |
| C10ORF57 | 0.0209204 | −1.23127 |
| IGSF9 | 0.0179381 | −1.23159 |
| FAM46B | 0.026685 | −1.23164 |
| LOC201229 | 0.00103037 | −1.23188 |
| PCDH19 | 0.00602381 | −1.23317 |
| CCDC92 | 0.0123843 | −1.23378 |
| IER5 | 0.00624369 | −1.23455 |
| BCL2L2 | 0.0238018 | −1.23484 |
| ADCY6 | 0.0456385 | −1.23491 |
| CDC42EP2 | 0.0233931 | −1.23509 |
| SFRS2B | 0.00217544 | −1.23521 |
| LOC388564 | 0.0209949 | −1.23595 |
| ITGA3 | 0.0128679 | −1.23596 |
| ZNF787 | 0.0493368 | −1.23732 |
| COX7A2L | 0.00917741 | −1.2376 |
| LOC729843 | 0.0210508 | −1.23824 |
| MAP4K1 | 0.0003889 | −1.23842 |
| YPEL3 | 0.0182327 | −1.2386 |
| KIAA1199 | 0.000438868 | −1.2394 |
| LAD1 | 0.00559147 | −1.23974 |
| PCK2 | 0.0469869 | −1.24051 |
| LRRC26 | 0.0263455 | −1.24134 |
| XPC | 0.0461144 | −1.2427 |
| GADD45A | 0.00539876 | −1.24378 |
| HS.535392 | 0.0112557 | −1.24395 |
| NINJ1 | 0.0220921 | −1.24426 |
| ATP9A | 0.0000423 | −1.24465 |
| APPL2 | 0.0184502 | −1.2451 |
| FAM134C | 0.0117325 | −1.24537 |
| KHDC1 | 0.0136015 | −1.24658 |
| RPUSD4 | 0.0129461 | −1.2466 |
| LOC100131735 | 0.039712 | −1.24766 |
| SNTB1 | 0.000265298 | −1.24826 |
| KIAA0363 | 0.00888731 | −1.24925 |
| GALM | 0.00133692 | −1.24945 |
| LOC100131713 | 0.0201168 | −1.24966 |
| LOC149501 | 0.00697955 | −1.25042 |
| ALDH3B2 | 0.0215538 | −1.25135 |
| TMEM118 | 0.00206767 | −1.25214 |
| C1ORF53 | 0.0122974 | −1.25234 |
| KDM5B | 0.0204729 | −1.25468 |
| LRRC56 | 0.0189076 | −1.25472 |
| CANT1 | 0.0426447 | −1.25579 |
| LMO1 | 0.0112807 | −1.25605 |
| SBK1 | 0.00158606 | −1.25664 |
| C11ORF52 | 0.00148247 | −1.25688 |
| COQ9 | 0.000919023 | −1.25794 |
| CLSTN1 | 0.0487511 | −1.25828 |
| LOC391578 | 0.032575 | −1.25871 |
| LOC646723 | 0.0250919 | −1.26072 |
| MFSD6 | 0.0161252 | −1.26073 |
| XBP1 | 0.00729036 | −1.26208 |
| ZNF444 | 0.0309465 | −1.26245 |
| KLHDC8B | 0.0338394 | −1.26265 |
| GSTT1 | 0.0474357 | −1.26269 |
| LOC647169 | 0.00621113 | −1.26395 |
| HIST1H2BK | 0.0147841 | −1.26493 |
| CALHM2 | 0.0198102 | −1.26515 |
| HIST3H2A | 0.0160256 | −1.26577 |
| CLIC1 | 0.0262659 | −1.26629 |
| RHOC | 0.00599548 | −1.26716 |
| WBSCR27 | 0.00794489 | −1.26732 |
| ACTA2 | 0.00996963 | −1.2676 |
| MTMR11 | 0.000436384 | −1.26844 |
| LOC387825 | 0.00115638 | −1.26929 |
| CDC25B | 0.0359915 | −1.2693 |
| GBP2 | 0.000258461 | −1.26993 |
| CD2BP2 | 0.0134025 | −1.27032 |
| LOC340598 | 0.00198138 | −1.27145 |
| PLA2G4B | 0.0453512 | −1.27164 |
| DDR1 | 0.0433909 | −1.27213 |
| ARHGEF16 | 0.0327749 | −1.27282 |
| TMEM98 | 0.013567 | −1.27308 |
| SLPI | 0.0230802 | −1.27351 |
| ARHGEF3 | 0.0010847 | −1.27482 |
| FAIM2 | 0.00257402 | −1.27505 |
| MDK | 0.010813 | −1.27522 |
| MALL | 0.00364316 | −1.27556 |
| SPATA20 | 0.00576647 | −1.27589 |
| CD44 | 0.0262195 | −1.27646 |
| LOC645381 | 0.0013696 | −1.27656 |
| ZFP36L1 | 0.0483853 | −1.2771 |
| FYCO1 | 0.00131353 | −1.27779 |
| EFNB3 | 0.0368701 | −1.27833 |
| FLJ10986 | 0.00464185 | −1.27873 |
| TUFT1 | 0.00743636 | −1.2788 |
| LOC388076 | 0.0398763 | −1.2793 |
| CEBPA | 0.0189943 | −1.28008 |
| DNAJC22 | 0.0123344 | −1.28079 |
| LOC678655 | 0.00259357 | −1.28173 |
| SERPINB1 | 0.0122474 | −1.28227 |
| C10ORF65 | 0.0000760 | −1.28273 |
| PRDM8 | 0.00172766 | −1.28273 |
| LAMB3 | 0.00506516 | −1.28414 |
| PPL | 0.00309682 | −1.28444 |
| CIB2 | 0.0122996 | −1.28474 |
| POMGNT1 | 0.00951761 | −1.28481 |
| KLHDC9 | 0.0208068 | −1.2854 |
| PPP1R1B | 0.00355738 | −1.2866 |
| FOXJ3 | 0.0381483 | −1.28928 |
| FAM43A | 0.00305297 | −1.29195 |
| C14ORF93 | 0.0261743 | −1.29226 |
| SNAPIN | 0.0018097 | −1.29312 |
| TLE1 | 0.0149653 | −1.29322 |
| ERGIC3 | 0.0137241 | −1.29454 |
| PLCXD1 | 0.0207925 | −1.29544 |
| FUCA1 | 0.00165054 | −1.29553 |
| EMP1 | 0.0232723 | −1.29667 |
| LOC391833 | 0.00762772 | −1.29696 |
| LOC100128098 | 0.00133192 | −1.30124 |
| ATF4 | 0.000145011 | −1.30182 |
| MYOF | 0.00478046 | −1.30343 |
| OCIAD2 | 0.0485847 | −1.30394 |
| SAT2 | 0.00334547 | −1.30522 |
| EFHD1 | 0.0131086 | −1.30543 |
| ZNF185 | 0.00361547 | −1.30743 |
| BCL3 | 0.00090822 | −1.30898 |
| PRMT6 | 0.0209409 | −1.30955 |
| SLC22A18 | 0.0205101 | −1.30974 |
| DBNDD1 | 0.0463087 | −1.31001 |
| COMMD7 | 0.00211526 | −1.31208 |
| ATP6V0E2 | 0.00168027 | −1.31212 |
| SLC2A10 | 0.00349165 | −1.31341 |
| FXYD3 | 0.000121051 | −1.31365 |
| VASN | 0.000305258 | −1.31379 |
| FER1L3 | 0.00886468 | −1.31542 |
| LOC643856 | 0.00332778 | −1.31691 |
| ZBTB42 | 0.00914152 | −1.31735 |
| FOXD2 | 0.00208269 | −1.31761 |
| PRRT3 | 0.000275288 | −1.31916 |
| LIMA1 | 0.00743353 | −1.32049 |
| DKK3 | 0.0000866 | −1.32333 |
| CRIP1 | 0.00750404 | −1.3234 |
| ACOX2 | 0.0144771 | −1.32483 |
| CAPN5 | 0.00665538 | −1.32545 |
| PIK3IP1 | 0.00421186 | −1.32796 |
| HS.28456 | 0.000648454 | −1.32861 |
| C14ORF78 | 0.00169572 | −1.33152 |
| FGD3 | 0.0175663 | −1.33747 |
| TNFRSF11B | 0.000549906 | −1.33838 |
| LRP10 | 0.0170834 | −1.33921 |
| DUSP6 | 0.00113798 | −1.3399 |
| S100A4 | 0.00408795 | −1.34038 |
| FOXQ1 | 0.00101956 | −1.34484 |
| SMAGP | 0.00219102 | −1.34516 |
| TRIB1 | 0.0000546 | −1.34525 |
| BLOC1S1 | 0.047093 | −1.34615 |
| MMP10 | 0.00796963 | −1.34687 |
| HEBP2 | 0.00721763 | −1.34712 |
| LMO4 | 0.00000227 | −1.34784 |
| CCDC102A | 0.0180642 | −1.3491 |
| LITAF | 0.0000571 | −1.35155 |
| PPAP2B | 0.0000191 | −1.35335 |
| EPHX1 | 0.00421714 | −1.35346 |
| FOXA1 | 0.0175729 | −1.35355 |
| GPR162 | 0.00209186 | −1.35466 |
| C1ORF106 | 0.00966517 | −1.35502 |
| CLCNKA | 0.0148036 | −1.35509 |
| FAM108C1 | 0.000371971 | −1.35527 |
| NCRNA00219 | 0.00443411 | −1.35724 |
| CDKN1A | 0.00643522 | −1.3575 |
| CNKSR3 | 0.000755548 | −1.35763 |
| BAIAP2L2 | 0.00181141 | −1.35858 |
| AKR7A3 | 0.00342655 | −1.36118 |
| TRIB3 | 0.0101274 | −1.36145 |
| ID2 | 0.0276121 | −1.36149 |
| BMP2 | 0.000258674 | −1.3669 |
| MT1G | 0.030306 | −1.367 |
| LOC650515 | 0.000833997 | −1.36751 |
| TMEM106C | 0.0151188 | −1.36865 |
| PPFIBP2 | 0.00030168 | −1.36875 |
| SOX4 | 0.00525706 | −1.3701 |
| TGFB3 | 0.000222326 | −1.37087 |
| MARCKSL1 | 0.0194441 | −1.37192 |
| MGST3 | 0.00000633 | −1.37452 |
| LOC124220 | 0.0112437 | −1.37485 |
| HOXB7 | 0.000117225 | −1.37675 |
| SYTL1 | 0.0204229 | −1.3781 |
| FAM167A | 0.0102848 | −1.37893 |
| SPINT2 | 0.00222094 | −1.37996 |
| HBB | 0.0164333 | −1.38002 |
| TMEM132A | 0.0281734 | −1.38198 |
| TIGA1 | 0.00264854 | −1.3829 |
| GPR56 | 0.000159323 | −1.38945 |
| TEAD2 | 0.00119516 | −1.38974 |
| PCOLCE | 0.0385339 | −1.39014 |
| SLC9A1 | 0.00675845 | −1.39175 |
| CMBL | 0.00217818 | −1.3934 |
| DBNDD2 | 0.00234655 | −1.39365 |
| CLDN7 | 0.00205505 | −1.39622 |
| CRIP2 | 0.0032777 | −1.39684 |
| KAZALD1 | 0.000278509 | −1.39847 |
| ID1 | 0.0035866 | −1.4048 |
| C1ORF64 | 0.000335664 | −1.40493 |
| MEGF6 | 0.00000815 | −1.4053 |
| CYP1A1 | 0.0025196 | −1.40861 |
| LOC340274 | 0.000953376 | −1.41461 |
| TM4SF5 | 0.00399629 | −1.41659 |
| LOC399965 | 0.00615872 | −1.4192 |
| FAM84B | 0.00523781 | −1.42345 |
| SH3GLB2 | 0.00160481 | −1.42692 |
| FLJ40504 | 0.00180029 | −1.43143 |
| ERBB3 | 0.0000973 | −1.4339 |
| ADM2 | 0.000383514 | −1.43644 |
| MARCKS | 0.00861746 | −1.43668 |
| TMEM125 | 0.0000286 | −1.44054 |
| PRPH | 0.00213604 | −1.4414 |
| S100A16 | 0.000433875 | −1.44268 |
| LOC440585 | 0.00314262 | −1.44415 |
| SERPINF1 | 0.00767474 | −1.44556 |
| RNASE1 | 0.0000973 | −1.45063 |
| GPR177 | 0.00000315 | −1.45118 |
| CA11 | 0.000353971 | −1.45137 |
| NRP1 | 0.00000418 | −1.45229 |
| C19ORF21 | 0.0000403 | −1.45229 |
| LAMA5 | 0.0248843 | −1.45281 |
| VIPR1 | 0.00012132 | −1.45703 |
| RAB3IP | 0.000138066 | −1.45728 |
| MLPH | 0.0000266 | −1.45942 |
| JUP | 0.0057439 | −1.46624 |
| CRABP2 | 0.000961909 | −1.46714 |
| SDC4 | 0.0102265 | −1.46902 |
| RDH5 | 0.000881998 | −1.46917 |
| TJP3 | 0.000300836 | −1.478 |
| AHNAK2 | 0.00977316 | −1.47968 |
| FLJ10916 | 0.000730181 | −1.48111 |
| CREB3L4 | 0.0000246 | −1.48148 |
| PTH2 | 0.0076484 | −1.48647 |
| GSTA2 | 0.00263022 | −1.48648 |
| C8ORF13 | 0.00443679 | −1.48703 |
| TGFBI | 0.0214205 | −1.48941 |
| LOC392871 | 0.00432192 | −1.48972 |
| SNHG7 | 0.0101863 | −1.49044 |
| HSPB8 | 0.00612702 | −1.49651 |
| BMP7 | 0.00196455 | −1.49653 |
| ALDH3A1 | 0.0035856 | −1.49736 |
| CHRM1 | 0.000188706 | −1.49911 |
| FLJ22184 | 0.000154193 | −1.50255 |
| PGAM2 | 0.0173066 | −1.50264 |
| SELENBP1 | 0.0040332 | −1.50467 |
| ALPPL2 | 0.000318459 | −1.50639 |
| KIAA1671 | 0.00165862 | −1.50665 |
| LUM | 0.0336263 | −1.51265 |
| EFNA1 | 0.000595979 | −1.51779 |
| SSBP2 | 0.0152658 | −1.5197 |
| IFNGR2 | 0.0000311 | −1.52504 |
| LOC644743 | 0.000673823 | −1.52702 |
| CBLN2 | 0.000226807 | −1.53323 |
| DDAH2 | 0.0000253 | −1.53651 |
| KRT8 | 0.000350867 | −1.54273 |
| CD9 | 0.000396975 | −1.56418 |
| C19ORF33 | 0.023951 | −1.57139 |
| LOC728910 | 0.0162248 | −1.57281 |
| P8 | 0.0192958 | −1.58139 |
| LOC100134170 | 0.00321867 | −1.58285 |
| PROM2 | 0.000773101 | −1.59159 |
| IGFBP2 | 0.000616956 | −1.59571 |
| LOC653499 | 0.00265122 | −1.59612 |
| FAM113B | 0.0000299 | −1.59652 |
| SOX2 | 0.000760904 | −1.59666 |
| MT1A | 0.0120414 | −1.59763 |
| MVP | 0.000235593 | −1.60537 |
| HMGCS2 | 0.0000400 | −1.61148 |
| C10ORF27 | 0.0319127 | −1.61265 |
| GSTA1 | 0.00435082 | −1.61554 |
| MT1F | 0.000408811 | −1.61567 |
| CAMK2N1 | 0.00224716 | −1.61865 |
| KRT18P13 | 0.00125645 | −1.62302 |
| MB | 0.000167216 | −1.6381 |
| LGALS4 | 0.00250822 | −1.63998 |
| C14ORF4 | 0.00446487 | −1.64667 |
| LGALS7 | 0.00572556 | −1.6539 |
| FOLR1 | 0.0000558 | −1.66099 |
| GDF15 | 0.0282679 | −1.67108 |
| ASS1 | 0.0000424 | −1.68142 |
| GSTA4 | 0.00714907 | −1.68702 |
| C17ORF28 | 0.00000850 | −1.69098 |
| CD24 | 0.000243734 | −1.69621 |
| ALG1L | 0.000157 | −1.70206 |
| LGALS7B | 0.0132446 | −1.71083 |
| MT2A | 0.0122104 | −1.72029 |
| GSTA3 | 0.0000366 | −1.72914 |
| KIAA1324 | 0.0000779 | −1.73151 |
| CDH1 | 0.000192229 | −1.73577 |
| KRT19 | 0.0015704 | −1.74865 |
| SCNN1A | 0.000132352 | −1.76004 |
| ATP1B1 | 0.000143511 | −1.76301 |
| PNPLA7 | 0.00028849 | −1.76318 |
| NUPR1 | 0.014055 | −1.77317 |
| GPNMB | 0.000179343 | −1.80085 |
| CAPS | 0.00000377 | −1.84113 |
| LOC387882 | 0.0000960 | −1.87876 |
| ZNF467 | 0.00121938 | −1.90275 |
| PRSS23 | 0.0101445 | −1.90499 |
| ABCC3 | 0.000000145 | −1.95032 |
| ALDH1A3 | 0.00306315 | −1.96769 |
| C1ORF115 | 0.00000493 | −1.99742 |
| IGFBP3 | 0.000000208 | −2.00474 |
| TSPAN1 | 0.000644243 | −2.01438 |
| TOB1 | 0.00108597 | −2.02797 |
| MGP | 0.00390921 | −2.06028 |
| TNFRSF21 | 0.0000644 | −2.08403 |
| ALDH3A2 | 0.0174888 | −2.12544 |
| C9ORF152 | 0.000228296 | −2.22537 |
| LFNG | 0.00000299 | −2.39135 |
| SCGB1D2 | 0.017697 | −2.41984 |
| NR2F1 | 0.0000373 | −2.49957 |
| ID3 | 0.000108394 | −2.79804 |
| TWIST1 | 0.00000150 | −2.8137 |
| PRODH | 0.000000765 | −2.87544 |
| SCGB2A2 | 0.0117456 | −3.00459 |

Genes up-regulated by AR variants in CWR-R1 cells transfected
with siRNAs targeting AR exon 1 vs. AR exon 7

| Probeset | p-value (siARExon7 | Fold-Change (siARExon7 |
|---|---|---|
| ID | vs. siARExon1) | vs. siARExon1) |
| NPTX2 | 0.00000392 | 3.68331 |
| APOD | 0.000156079 | 3.03322 |
| FOSB | 0.0229884 | 1.77893 |
| C20ORF134 | 0.0188557 | 1.76078 |
| FLJ35767 | 0.0132673 | 1.70458 |
| IL12A | 0.00018406 | 1.52885 |
| CAMKV | 0.0304333 | 1.52226 |
| TMEM145 | 0.00362716 | 1.5218 |
| CSRNP2 | 0.0248431 | 1.44072 |
| NFE2L1 | 0.0379217 | 1.41858 |
| FSCN1 | 0.0403215 | 1.41697 |
| UNC119 | 0.00578944 | 1.40844 |
| GNPDA1 | 0.00222167 | 1.39344 |
| LRRC8A | 0.0258392 | 1.37538 |
| HEY1 | 0.0241776 | 1.35489 |
| NRBP2 | 0.00395238 | 1.35162 |
| JAM3 | 0.0454343 | 1.34512 |
| DUSP3 | 0.0324714 | 1.33968 |
| SRP68 | 0.0162508 | 1.33098 |
| BHLHB2 | 0.0072524 | 1.33074 |
| ALDOC | 0.0212065 | 1.32869 |
| ELAVL1 | 0.0246166 | 1.32036 |
| ACLY | 0.037078 | 1.31674 |
| GAGE12C | 0.0499085 | 1.31671 |
| INSIG1 | 0.0274392 | 1.30547 |
| STK39 | 0.0297507 | 1.30432 |
| AGT | 0.00270182 | 1.29234 |
| LONRF1 | 0.0144737 | 1.28517 |
| PROCR | 0.019772 | 1.27797 |
| SNCA | 0.00472685 | 1.27489 |
| CYP27B1 | 0.0256534 | 1.27039 |
| SFXN1 | 0.0336174 | 1.26436 |
| NT5DC3 | 0.0460989 | 1.25298 |
| PDCD4 | 0.00148145 | 1.24993 |
| SLC31A2 | 0.00539321 | 1.24954 |
| ALAS1 | 0.0435645 | 1.24913 |
| ACOT7 | 0.046092 | 1.24536 |
| CD164L2 | 0.0494376 | 1.24484 |
| PEG10 | 0.0190778 | 1.2439 |
| NFYA | 0.0186035 | 1.2404 |
| GSTM1 | 0.0240796 | 1.23849 |
| RNU6-15 | 0.0384087 | 1.23792 |
| FKBP5 | 0.0482986 | 1.23194 |
| MED30 | 0.0163048 | 1.22969 |
| LRP8 | 0.0147108 | 1.22965 |
| SLC2A12 | 0.0128015 | 1.22632 |
| ELF3 | 0.0176265 | 1.22529 |
| ULBP2 | 0.0232297 | 1.22501 |
| PAICS | 0.039816 | 1.22373 |
| SLC25A4 | 0.00432368 | 1.22323 |
| THBS1 | 0.0113489 | 1.22101 |
| IRS2 | 0.00973364 | 1.21649 |
| PDIA5 | 0.0397258 | 1.21454 |
| LOC387763 | 0.00926292 | 1.21406 |
| MEF2D | 0.0312234 | 1.21315 |
| HS.582526 | 0.00962906 | 1.20697 |
| MLX | 0.0473073 | 1.20101 |
| CDC42EP2 | 0.0422471 | −1.20008 |
| SARS | 0.00764572 | −1.20067 |
| LOC647436 | 0.00182389 | −1.20182 |
| CD9 | 0.0434272 | −1.20233 |
| IGSF9 | 0.0300475 | −1.20298 |
| FLJ10916 | 0.0373263 | −1.20305 |
| C2ORF79 | 0.00407883 | −1.20547 |
| C1ORF64 | 0.0111944 | −1.20548 |
| LOC390557 | 0.0175923 | −1.20593 |
| PRDM8 | 0.00834572 | −1.20653 |
| LOC346950 | 0.00397147 | −1.20715 |
| SH3GLB2 | 0.0373035 | −1.20912 |
| CIB2 | 0.040609 | −1.20925 |
| VIPR1 | 0.00811547 | −1.2093 |
| FAM108C1 | 0.00603078 | −1.21108 |
| LOC100133328 | 0.0240931 | −1.21247 |
| CHST15 | 0.00194298 | −1.21574 |
| DKK3 | 0.000960304 | −1.21602 |
| CLDN3 | 0.0161007 | −1.22151 |
| TIMM10 | 0.0375553 | −1.2236 |
| LPAR5 | 0.0199587 | −1.22468 |
| A2LD1 | 0.00281889 | −1.22469 |
| LOC100132717 | 0.0228325 | −1.22482 |
| HIST1H2BK | 0.0267734 | −1.22825 |
| B3GNT1 | 0.015751 | −1.22981 |
| ZNF837 | 0.0249236 | −1.23114 |
| LANCL2 | 0.0132031 | −1.23116 |
| PRAGMIN | 0.00660192 | −1.23224 |
| ID1 | 0.0367015 | −1.23265 |
| HNRNPA0 | 0.0290863 | −1.23342 |
| C19ORF21 | 0.00186906 | −1.23368 |
| TLE1 | 0.0336897 | −1.23763 |
| TMEM79 | 0.0134838 | −1.23997 |
| HMGCS2 | 0.00625569 | −1.2418 |
| ATP6V0E2 | 0.00573956 | −1.24485 |
| C1ORF53 | 0.012692 | −1.2505 |
| MB | 0.0168336 | −1.25183 |
| LOC389816 | 0.0167034 | −1.25416 |
| PRMT6 | 0.0411402 | −1.25697 |
| CAPS | 0.00294816 | −1.25955 |
| ZBTB42 | 0.0198642 | −1.26372 |
| TNFRSF21 | 0.0407956 | −1.2663 |
| MT1F | 0.0206399 | −1.26897 |
| FAM136A | 0.0385671 | −1.26954 |
| POLR2L | 0.0164873 | −1.27176 |
| TRIB3 | 0.0302818 | −1.27412 |
| PSCA | 0.0148045 | −1.27433 |
| CHRM1 | 0.00422523 | −1.27924 |
| DYNLL1 | 0.0298593 | −1.27928 |
| KRT8 | 0.00974294 | −1.27982 |
| FOLR1 | 0.00534994 | −1.28198 |
| SLPI | 0.0200907 | −1.28346 |
| GSTA3 | 0.00536325 | −1.28746 |
| LOC388564 | 0.00902696 | −1.28818 |
| CLDN7 | 0.00913333 | −1.28986 |
| C11ORF80 | 0.00129074 | −1.29074 |
| FLJ22184 | 0.00301421 | −1.29089 |
| AKR7A3 | 0.00910645 | −1.29299 |
| GSTA2 | 0.0229884 | −1.29553 |
| MMP10 | 0.0154419 | −1.29714 |
| C17ORF28 | 0.00111574 | −1.29729 |
| CAMK2N1 | 0.0432565 | −1.29924 |
| C1ORF115 | 0.00348662 | −1.30104 |
| KRT18P13 | 0.0295819 | −1.30132 |
| ASS1 | 0.00355931 | −1.30164 |
| PNPLA7 | 0.020948 | −1.30519 |
| ALDH3A1 | 0.0274631 | −1.30595 |
| ETS1 | 0.0194391 | −1.31778 |
| TMEM125 | 0.000199305 | −1.31968 |
| KIAA1324 | 0.00564776 | −1.32162 |
| LOC340274 | 0.00349726 | −1.32195 |
| FLJ40504 | 0.00720201 | −1.32341 |
| ADM2 | 0.00184164 | −1.32657 |
| MVP | 0.00497518 | −1.33473 |
| LRRC26 | 0.00552524 | −1.34892 |
| HEBP2 | 0.00570156 | −1.36557 |
| KLK11 | 0.0488535 | −1.37646 |
| C10ORF116 | 0.0335648 | −1.38331 |
| GPNMB | 0.00624908 | −1.39158 |
| MT1X | 0.0486692 | −1.40585 |
| IGFBP3 | 0.0000455 | −1.40606 |
| TWIST1 | 0.00282283 | −1.41799 |
| MLPH | 0.0000438 | −1.42299 |
| PRODH | 0.00144588 | −1.4412 |
| NR2F1 | 0.0113011 | −1.44298 |
| ID3 | 0.0281163 | −1.47865 |
| C9ORF152 | 0.0109972 | −1.517 |
| LFNG | 0.000218976 | −1.62026 |

### Example 3

### Next-Generation Paired-End Re-Sequencing of the 183 Kb AR Gene

Genomic DNA from LuCaP 136 xenograft tumor tissue was subjected to hybrid capture with a custom SureSelect bait library (Table 3, Agilent, Santa Clara, Calif.) and sequenced at 6000× depth with 2×100 bp settings on an Illumina HiSeq 2000 (Illumina, Inc., San Diego, Calif.) as described (Li et al., 2012. Oncogene 31(45):4759-4767) using primers identified in Table 5. Briefly, raw sequence data from HiSeq 2000 was de-multiplexed and filtered using CASAVA 1.8 (Illumina, Inc., San Diego, Calif.). FASTQ formatted reads were inspected using fastQC (Babraham Institute, Cambridge, United Kingdom). Filtered reads were trimmed (from an initial length of 2×100 bp to 2×85 bp, removing sequence from the 3′ end) to remove low-quality ends using the FASTQ trimmer tool in Galaxy, then mapped to the hg19 build of the reference genome using Burrows-Wheeler Alignment (BWA) (Li, H. and Durbin, R. 2009. Bioinformatics 25(14):1754-1760). Briefly, for BWA alignment, the seed size was 20 (“−120”). Up to two differences within the seed (“−k 2”), up to 4 differences in each read end (“−n 4”), and up to 1 gap opening in the alignment (“−o 1”) were allowed. In the alignment pairing phase, the maximal expected insert size (“−a”) was set to be 500. Up to 10 million possible mapping locations (“−o 10000000”) were also allowed. Output BAM files from BWA were sorted, followed by removal of potential PCR duplicates using Picard tools (picard.sourceforge.net/). Discordantly-mapped read pairs and soft-clip reads were collected and used to identify structural variations via LUMPY (github.com/arq5x/lumpy-sv) with parameters: −mw 3, −tt 1e-3, back_distance:20, weight:1, min_non_overlap:85, discordant_z:7, back_distance:20, mean:227, stdev:73.

### RT-PCR and Quantitative RT-PCR.

RNA was isolated and converted to cDNA as described previously (Li et al. 2011. Cancer Res 71(6):2108-2117). cDNA was used for PCR reactions with exon 4 fwd and exon 8 rev primers (Table 5) using Taq DNA polymerase (Qiagen Inc. USA, Valencia, Calif.) according to the manufacturer's protocol. For quantitative RT-PCR assays, cDNA was used for quantitative PCR reactions with primers specific for FKBP5, LIMA1, FASN, and GAPDH (Table 5) using a BioRad iCycler™ (Bio-Rad Laboratories, Hercules, Calif.) and PerfeCTa Sybr Green FastMix (Quanta Biosciences, Inc., Gaithersburg, Md.) according to the manufacturer's protocol.

TABLE 5

| | | |
|---|---|---|
| | | SEQ |
| Primer | Sequence | ID NO: |

LuCap 86.2 deletion PCR (FIG. 24A)

| | | |
|---|---|---|
| qdel F2 | 5′-TAGGGTTGCAGCTACTCTTTCC | 1439 |
| qdel R3 | 5′-TGCTTAGCACTCAAACCCAGTA | 1440 |
| nor F | 5′-TAGGGTTGCAGCTACTCTTTCC | 1439 |
| | (same as qdel F2) | |
| nor R2 | 5′-TGTGTGTTAGAGAGAGACAGCGA | 1441 |

LuCap 136 inversion PCR (FIG. 24B)

| | | |
|---|---|---|
| 136inv int4+ | 5′-ATTTGGAGTGGGTGAGTAGACTGG | 1442 |
| 136inv int7+ | 5′-TGCTTTTATCAGGGAGAACAGCC | 1443 |
| 136inv int4− | 5′-AGCTCTCTGACTCAGACTTC | 1444 |
| 136inv int7− | 5′-GTGAATGTGAAGGCACATGG | 1445 |

LuCap 136 inversion quantitative PCR

| | | |
|---|---|---|
| qLuCap 136 F1 | 5′-CCACTTGCCTTGCCTAGAAG | 1446 |
| qLuCap 136 R1 | 5′-GGGTGGAGGAGTTGAGAACA | 1447 |
| qAR normal R1 | 5′-CAAAGAAAGGCCAGTTTGGA | 1448 |
| qAR intron1 F | 5′-TGGATGGATAGCTACTCCGG | 1449 |
| qAR intron1 R | 5′-TTTACCCTGCTGAGCTGTCC | 1450 |

AR RT-PCR

| | | |
|---|---|---|
| EXON4 F | 5′-GCAGCAAAGATTTCCAAACTGG | 1451 |
| EXON8 R | 5′-TGGGTGTGGAAATAGATGGG | 1452 |

qRT-PCR

| | | |
|---|---|---|
| qpcr-GAPDH F | 5′-GAAGGTGAAGGTCGGAGTC | 1453 |
| qpcr-GAPDH R | 5′-GAAGATGGTGATGGGATTTC | 1454 |
| qpcr-FKBP5 F | 5′-AGGAGGGAAGAGTCCCAGTG | 1455 |
| qpcr-FKBP5 R | 5′-TGGGAAGCTACTGGTTTTGC | 1456 |
| FASN F1 | 5′-CTGAAGCGTGGCCTGAAG | 1457 |
| FASN F2 | 5′-CTTCCTCACCTCCACTGAGC | 1458 |
| LIMA1 R1 | 5′-TTTTGCTTGCCCATAGATCC | 1459 |
| APIP F1 | 5′-ACTGGGACTGGAGGAGGAAT | 1460 |
| APIP R1 | 5′-ATCACTGCACCTGCTCCTCT | 1461 |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method for detecting expression of an androgen receptor (AR) variant, the method comprising:
receiving a biological sample obtained from a subject, the biological sample comprising cells expressing a plurality of non-wild-type androgen receptor polynucleotides, each non-wild-type androgen receptor polynucleotide being encoded by a genomic polynucleotide comprising a copy number;
measuring the copy number of at least one genomic polynucleotide that, when transcribed, produces a non-wild-type androgen receptor polynucleotide; and
identifying the sample as exhibiting expression of an AR variant if the at least one genomic polynucleotide exhibits a copy number that differs from the mean AR copy number by at least one standard deviation.

2. The method of claim 1 wherein the non-wild-type androgen receptor polynucleotide comprises at least a portion of AR intron 1.

3. The method of claim 2 wherein at least a portion of AR intron 1 exhibits a copy number that is greater than the mean AR copy number by at least one standard deviation.

4. The method of claim 1 wherein at least a portion of AR intron 1 exhibits a copy number that is less than the mean AR copy number by at least one standard deviation.

5. The method of claim 4 wherein the non-wild-type androgen receptor polynucleotide exhibits a 48,476 bp deletion from AR intron 1.

6. The method of claim 1 wherein the non-wild-type androgen receptor polynucleotide exhibits a deletion of at least a portion of AR exon 5, AR exon 6, or AR exon 7.

7. The method of claim 6 wherein the non-wild-type androgen receptor polynucleotide exhibits an 8579 bp deletion of AR exon 5, AR exon 6, and a portion of AR exon 7.

8. The method of claim 1 wherein the subject has received treatment for prostate cancer.

9. The method of claim 1 further comprising identifying the subject as at risk for castration-resistant prostate cancer.

10. The method of claim 9 further comprising either initiating or modifying treatment of the subject at risk for castration-resistant prostate cancer.

11. The method of claim 10 wherein initiating or modifying treatment comprises administering to the subject at least one pharmaceutical composition effective for treating castration-resistant prostate cancer.

12. The method of claim 11 wherein the pharmaceutical composition effective for treating castration-resistant prostate cancer comprises radium-233 dichloride, an immunotherapy, a taxane, a kinase inhibitor, an AR amino-terminal domain inhibitor, or an AR DNA binding domain inhibitor.

13. The method of claim 10 wherein modifying treatment of the subject at risk for castration-resistant prostate cancer comprises decreasing one or more composnnts of androgen depletion therapy.

14. A method comprising:
administering to a subject at risk of developing castration-resistant prostate cancer a composition that comprises an inhibitor of an androgen receptor (AR) splice variant associated with castration-resistant prostate cancer.

15. The method of claim 14 wherein the inhibitor comprises a polynucleotide that hybridizes to at least a portion of a transcript of the AR splice variant.

16. The method of claim 15 wherein the polynucleotide comprises a siRNA.

17. The method of claim 14 wherein the AR splice variant comprises at least a portion of AR exon 1 or at least a portion of AR exon 7.

18. The method of claim 14 wherein the splice variant comprises AR 1/2/2b, AR 1/2/3/2b, AR 1/2/3/CE1, AR 1/2/3/CE2, AR 1/2/3/CE3, or ARv567es.

19. A method for detecting expression of an androgen receptor (AR) variant, the method comprising:
receiving a biological sample obtained from a subject, the biological sample comprising at least one cell comprising an AR variant comprising a linear rearrangement of AR genomic DNA;
sequencing a sufficient portion of the AR genomic DNA to detect the linear rearrangement of the AR genomic DNA; and
detecting the linear rearrangement of AR genomic DNA.

20. The method of claim 19 wherein sequencing the portion of the AR genomic DNA comprises:
fragmenting the subject's genomic DNA;
hybridizing at least a portion of the fragmented genomic DNA to a polynucleotide complementary to at least a portion of the AR genomic DNA;
separating hybridized genomic DNA from non-hybridized genomic DNA;
amplifying the hybridized genomic DNA; and
sequencing the amplified genomic DNA.

21. The method of claim 19 wherein the subject has received treatment for prostate cancer.

22. The method of claim 19 further comprising identifying the subject as at risk for castration-resistant prostate cancer.

23. The method of claim 22 further comprising either initiating or modifying treatment of the subject at risk for castration-resistant prostate cancer.

24. The method of claim 23 wherein initiating or modifying treatment comprises administering to the subject at least one pharmaceutical composition effective for treating castration-resistant prostate cancer.

25. The method of claim 24 wherein the pharmaceutical composition effective for treating castration-resistant prostate cancer comprises radium-233 dichloride, an immunotherapy, a taxane, a kinase inhibitor, an AR amino-terminal domain inhibitor, or an AR DNA binding domain inhibitor.

26. The method of claim 23 wherein modifying treatment of the subject at risk for castration-resistant prostate cancer comprises decreasing one or more components of androgen depletion therapy.

27. The method of claim 19 wherein the linear rearrangement of the AR genomic DNA exhibits a deletion of at least a portion of AR intron 1.

28. The method of claim 19 wherein the linear rearrangement of the AR genomic DNA exhibits a deletion of at least a portion of AR exon 5, AR exon 6, or AR exon 7.

29. The method of claim 19 wherein the linear rearrangement of the AR genomic DNA exhibits an inversion of at least a portion of AR exon 5, AR exon 6, or AR exon 7.


SEQUENCE LISTING (1463 sequences)

SEQ ID NO: 1 - DNA, 19 bp, artificial
  siRNAs targeted to AR Exon 7
ggaacucgaucguaucauu

SEQ ID NO: 2 - DNA, 19 bp, artificial
  siRNAs targeted to AR Exon 1
caagggagguuacaccaaa

SEQ ID NO: 3 - DNA, 20 bp, artificial
  synthetic oligonucleotide primer
caaccccaacgtcaaagtct

SEQ ID NO: 4 - DNA, 20 bp, artificial
  synthetic oligonucleotide primer
ctgtgaccagggagaatggt

SEQ ID NO: 5 - DNA, 24 bp, artificial
  synthetic oligonucleotide primer
ttcagattaccaagtttcttcagc

SEQ ID NO: 6 - DNA, 20 bp, artificial
  synthetic oligonucleotide primer
gcaaattggaggcagaaatc

SEQ ID NO: 7 - DNA, 20 bp, artificial
  synthetic oligonucleotide primer
cccagctggtttaggaatca

SEQ ID NO: 8 - DNA, 20 bp, artificial
  synthetic oligonucleotide primer
tatgaaggagaagggccaga

SEQ ID NO: 9 - DNA, 20 bp, artificial
  synthetic oligonucleotide primer
atggccttttggtttgaatg

SEQ ID NO: 10 - DNA, 20 bp, artificial
  synthetic oligonucleotide primer
cggaagctgaagaaacttgg

SEQ ID NO: 11 - DNA, 20 bp, artificial
  synthetic oligonucleotide primer
tgggtgtggaaatagatggg

SEQ ID NO: 12 - DNA, 22 bp, artificial
  synthetic oligonucleotide primer
gcagcaaagatttccaaactgg

SEQ ID NO: 13 - DNA, 24 bp, artificial
  synthetic oligonucleotide primer
cctctgatttttggtctttcagcc

SEQ ID NO: 14 - DNA, 66 bp, artificial
  synthetic oligonucleotide probe
gggttccctaagggttggatctggacccgtgatggccgtgaattattgcttgcacactcatgggtg

SEQ ID NO: 15 - DNA, 70 bp, artificial
  synthetic oligonucleotide probe
atgctactccctctctctcatggcaattcttcattctctggttttcgtctagattggatcttgctggcac

SEQ ID NO: 16 - DNA, 58 bp, artificial
  synthetic oligonucleotide probe
gggttccctaagggttggatcggcgttaggatttccctgggaatggtgagcctccatt

SEQ ID NO: 17 - DNA, 62 bp, artificial
  synthetic oligonucleotide probe
gatggtttcaacacacagccaaggccctatcttccggaatctagattggatcttgctggcac

SEQ ID NO: 18 - DNA, 64 bp, artificial
  synthetic oligonucleotide probe
gggttccctaagggttggataaaaaactaccgtggtttggggttaaaccgtgagtaaccttatt

SEQ ID NO: 19 - DNA, 68 bp, artificial
  synthetic oligonucleotide probe
ttctaggtctcagccaactttgaagggcatggaaaagtcggtggatctagattggatcttgctggcac

SEQ ID NO: 20 - DNA, 96 bp, artificial
  synthetic oligonucleotide probe
gggttccctaagggttggagcaggaagtcgttaccacctggcccacggagccaatttctcatgcttggtcacaacgtttggagatagggaagagtt

SEQ ID NO: 21 - DNA, 100 bp, artificial
  synthetic oligonucleotide probe
tgtggatggatcatggcagtgcatggacagtgaaaacgtggtgtaccggctgtctggtatgtatgagtttgtggtgatctagattggatcttgctggcac

SEQ ID NO: 22 - DNA, 56 bp, artificial
  synthetic oligonucleotide probe
gggttccctaagggttggataggccgagctgtcctacgagtgtccagaatcctctg

SEQ ID NO: 23 - DNA, 60 bp, artificial
  synthetic oligonucleotide probe
tagtcttgggcctggtgcttgagagacccaaagcgagtctagattggatcttgctggcac

SEQ ID NO: 24 - DNA, 60 bp, artificial
  synthetic oligonucleotide probe
gggttccctaagggttggatgtgaatggggagtgacctgtcttgaattcagaactgcgca

SEQ ID NO: 25 - DNA, 64 bp, artificial
  synthetic oligonucleotide probe
gatcattccccattctaaggccctctcatgccggatgctaatctagattggatcttgctggcac

SEQ ID NO: 26 - DNA, 62 bp, artificial
  synthetic oligonucleotide probe
gggttccctaagggttggatcagcgcaacacaacctggtgatctcagctgggtgccaaggtt

SEQ ID NO: 27 - DNA, 66 bp, artificial
  synthetic oligonucleotide probe
tcctaagcccaagttccccatggttgagcctccttatctggtttctagattggatcttgctggcac

SEQ ID NO: 28 - DNA, 70 bp, artificial
  synthetic oligonucleotide probe
gggttccctaagggttggatgtgggaggcgaaaattggcaggtcaggatcagtgggagtgctacccaaaa

SEQ ID NO: 29 - DNA, 74 bp, artificial
  synthetic oligonucleotide probe
tattttgctagctggggagtcagggagaagcgaacgtccggatgctgaagttctagattggatcttgctggcac

SEQ ID NO: 30 - DNA, 100 bp, artificial
  synthetic oligonucleotide probe
gggttccctaagggttggaggtgacgagtgcggttatagcggtccggctgtcgcggatgaatatgaccaggccctctacctgaagatatcttgctactga

SEQ ID NO: 31 - DNA, 104 bp, artificial
  synthetic oligonucleotide probe
tgctgtctcacagtgtctgaaactcccatcagccaacgtccgatatcacgaaggataaatgcagcaaatgcctgagcggtttctagattggatcttgctggcac

SEQ ID NO: 32 - DNA, 76 bp, artificial
  synthetic oligonucleotide probe
gggttccctaagggttggatgcaggtcgaaaaatgggtggatggcacgaggggtttggaaatcagaaaaccagcag

SEQ ID NO: 33 - DNA, 80 bp, artificial
  synthetic oligonucleotide probe
aggcaggaaaactcagggcagcatgggagataggaaagcaatactctgggacacgtatctagattggatcttgctggcac

SEQ ID NO: 34 - DNA, 52 bp, artificial
  synthetic oligonucleotide probe
gggttccctaagggttggatgtccacaaggccatatgcttcctagacaaaga

SEQ ID NO: 35 - DNA, 56 bp, artificial
  synthetic oligonucleotide probe
gaaaagatttctgccacactcagaacgctttactctagattggatcttgctggcac

SEQ ID NO: 36 - DNA, 78 bp, artificial
  synthetic oligonucleotide probe
gggttccctaagggttggatattacgcccgtgccttatccggagaggtaggagaagtgacttggaagcagtcttgaga

SEQ ID NO: 37 - DNA, 82 bp, artificial
  synthetic oligonucleotide probe
gattgcctgttccatcccctatctttgtcctatgaatgacgcgacaggaagaacttgactctagattggatcttgctggcac

SEQ ID NO: 38 - DNA, 82 bp, artificial
  synthetic oligonucleotide probe
gggttccctaagggttggagagcggaaagagcattattcagcgcccgttcctgctcctcaacacagactttgacgttggggt

SEQ ID NO: 39 - DNA, 86 bp, artificial
  synthetic oligonucleotide probe
tgggggctactctcttgattgctgactcccttgaccgtgtggcttacctgaccgccggtatcgtctagattggatcttgctggcac

SEQ ID NO: 40 - DNA, 94 bp, artificial
  synthetic oligonucleotide probe
gggttccctaagggttggagtatggtggccagcggctatgactaccggcgcgacgatgatgcgtggagacttgggggaaagaatcaaggagcct

SEQ ID NO: 41 - DNA, 98 bp, artificial
  synthetic oligonucleotide probe
tcttgcctgggggaatttggcatgcacttatggcttgtggagttcagccgatctgacttatgtcattacctatgatctagattggatcttgctggcac

SEQ ID NO: 42 - DNA, 72 bp, artificial
  synthetic oligonucleotide probe
gggttccctaagggttggatgccacgacgatgaacagacgcaagccattgagccaggtgtagtgtgtgctgg

SEQ ID NO: 43 - DNA, 76 bp, artificial
  synthetic oligonucleotide probe
acacgacaacaaccagcccgactcctttgcatgctgcgtgtggatgaggccattctagattggatcttgctggcac

SEQ ID NO: 44 - DNA, 86 bp, artificial
  synthetic oligonucleotide probe
gggttccctaagggttggagtccaagggaccgagtgaaagtgtggatgcagccctaccagttcagcctctttctctctatcccagg

SEQ ID NO: 45 - DNA, 90 bp, artificial
  synthetic oligonucleotide probe
gaagccctaggtcactcttgcaaaatcttaggttgcccaactttacccgtggcaatgcccgcgcacatctagattggatcttgctggcac

SEQ ID NO: 46 - DNA, 54 bp, artificial
  synthetic oligonucleotide probe
gggttccctaagggttggatcaagctgagcacctcctcaaaatagaccctcagt

SEQ ID NO: 47 - DNA, 58 bp, artificial
  synthetic oligonucleotide probe
tatagccagatgcatctcgtgagccaggatgaaagtctagattggatcttgctggcac

SEQ ID NO: 48 - DNA, 90 bp, artificial
  synthetic oligonucleotide probe
gggttccctaagggttggagacattgttacactgtggaggagtccatgacgaaagatgaacaaagtcagcagggcaaactccactgtatc

SEQ ID NO: 49 - DNA, 94 bp, artificial
  synthetic oligonucleotide probe
tggtagagaatccagaagccaggccaagtctactgattgcccgtctccgctcgctgggtgaacaactgaactctagattggatcttgctggcac

SEQ ID NO: 50 - DNA, 80 bp, artificial
  synthetic oligonucleotide probe
gggttccctaagggttggatcggtgagacgtgggaggcgaaaattggcgcacccccaaacatatgggcattaccattcca

SEQ ID NO: 51 - DNA, 84 bp, artificial
  synthetic oligonucleotide probe
tcaccccctggagtgccaactatttgactgaaacgtccggatgctgaagtgatggcagagctctagattggatcttgctggcac

SEQ ID NO: 52 - DNA, 50 bp, artificial
  synthetic oligonucleotide probe
gggttccctaagggttggatcccattggaaatcaagctgggagagagctt

SEQ ID NO: 53 - DNA, 54 bp, artificial
  synthetic oligonucleotide probe
cctgtagcttcaccctttccacaggcgttgctctagattggatcttgctggcac

SEQ ID NO: 54 - DNA, 84 bp, artificial
  synthetic oligonucleotide probe
gggttccctaagggttggagactcccagctggaccgctacgaaatgcgcgtatatctttgttcctcccaacaccaatgccccac

SEQ ID NO: 55 - DNA, 88 bp, artificial
  synthetic oligonucleotide probe
tccaagttcccatttcccacctcttggatgcggggatgggggccgggtgaggaaagctggctgattctagattggatcttgctggcac

SEQ ID NO: 56 - DNA, 98 bp, artificial
  synthetic oligonucleotide probe
gggttccctaagggttggagatgcgtgatggtggcctgttctccggaggtggacgatgaagaccttcagtgagacctgaacatcagagggggactgag

SEQ ID NO: 57 - DNA, 102 bp, artificial
  synthetic oligonucleotide probe
tagtaagagtagctaggaggccacagactgccgctggaaagtgaaacccggtatggatgtggcttcggtcccttctgtatctagattggatcttgctggcac

SEQ ID NO: 58 - DNA, 92 bp, artificial
  synthetic oligonucleotide probe
gggttccctaagggttggagtgagcagtcaggtggcgtgatacgtggtgtttttgatgaccgcaagctcttcagcctcccttttctcccata

SEQ ID NO: 59 - DNA, 96 bp, artificial
  synthetic oligonucleotide probe
ttccaaaagttgcctttgggttcgtccactactgaaaatatcagctatgccggacagggcgtgcgcgttgaagtctagattggatcttgctggcac

SEQ ID NO: 60 - DNA, 68 bp, artificial
  synthetic oligonucleotide probe
gggttccctaagggttggatcatccggtgaagagattaggagacgacactgtagttttcacccgctgt

SEQ ID NO: 61 - DNA, 72 bp, artificial
  synthetic oligonucleotide probe
ggctgagatgttgccaaaatctgagatccctgagccacctgacagtgtgtctagattggatcttgctggcac

SEQ ID NO: 62 - DNA, 74 bp, artificial
  synthetic oligonucleotide probe
gggttccctaagggttggatctgacctttcacatctggacagccaccaactccaaaaccgtgggattctgcttt

SEQ ID NO: 63 - DNA, 78 bp, artificial
  synthetic oligonucleotide probe
tcatcctctgtggtgttggccagatcatactgtacagcccgttcagcacctgggttctagattggatcttgctggcac

SEQ ID NO: 64 - DNA, 120 bp, Homo sapiens
agctagccgctccagtgctgtacaggagccgaagggacgcaccacgccagccccagcccggctccagcgacagccaacgcctcttgcagcgcggcggcttcgaagccgccgcccggagct

SEQ ID NO: 65 - DNA, 120 bp, Homo sapiens
gcatttgctctccacctcccagcgccccctccgagatcccggggagccagcttgctgggagagcgggacggtccggagcaagcccagaggcagaggaggcgacagagggaaaaagggccg

SEQ ID NO: 66 - DNA, 120 bp, Homo sapiens
gctccagcgacagccaacgcctcttgcagcgcggcggcttcgaagccgccgcccggagctgccctttcctcttcggtgaagtttttaaaagctgctaaagactcggaggaagcaaggaaa

SEQ ID NO: 67 - DNA, 120 bp, Homo sapiens
gagcgggacggtccggagcaagcccagaggcagaggaggcgacagagggaaaaagggccgagctagccgctccagtgctgtacaggagccgaagggacgcaccacgccagccccagcccg

SEQ ID NO: 68 - DNA, 120 bp, Homo sapiens
acccgcctggttaggctgcacgcggagagaaccctctgttttcccccactctctctccacctcctcctgccttccccaccccgagtgcggagccagagatcaaaagatgaaaaggcagtc

SEQ ID NO: 69 - DNA, 120 bp, Homo sapiens
ttgcaaagaaggctcttaggagccaggcgactggggagcggcttcagcactgcagccacgacccgcctggttaggctgcacgcggagagaaccctctgttttcccccactctctctccac

SEQ ID NO: 70 - DNA, 120 bp, Homo sapiens
cagcccgagtttgcagagaggtaactccctttggctgcgagcgggcgagctagctgcacattgcaaagaaggctcttaggagccaggcgactggggagcggcttcagcactgcagccacg

SEQ ID NO: 71 - DNA, 120 bp, Homo sapiens
gaagggtctaccctcggccgccgtccaagacctaccgaggagctttccagaatctgttccagagcgtgcgcgaagtgatccagaacccgggccccaggcacccagaggccgcgagcgcag

SEQ ID NO: 72 - DNA, 120 bp, Homo sapiens
gtaagggaagtaggtggaagattcagccaagctcaaggatggaagtgcagttagggctgggaagggtctaccctcggccgccgtccaagacctaccgaggagctttccagaatctgttcc

SEQ ID NO: 73 - DNA, 120 bp, Homo sapiens
gatcttgtccaccgtgtgtcttcttctgcacgagactttgaggctgtcagagcgctttttgcgtggttgctcccgcaagtttccttctctggagcttcccgcaggtgggcagctagctgc

SEQ ID NO: 74 - DNA, 120 bp, Homo sapiens
agcgactaccgcatcatcacagcctgttgaactcttctgagcaagagaaggggaggcggggtaagggaagtaggtggaagattcagccaagctcaaggatggaagtgcagttagggctgg

SEQ ID NO: 75 - DNA, 120 bp, Homo sapiens
gcgtggttgctcccgcaagtttccttctctggagcttcccgcaggtgggcagctagctgcagcgactaccgcatcatcacagcctgttgaactcttctgagcaagagaaggggaggcggg

SEQ ID NO: 76 - DNA, 120 bp, Homo sapiens
ctacttcagtggacactgaatttggaaggtggaggattttgtttttttcttttaagatctgggcatcttttgaatctacccttcaagtattaagagacagactgtgagcctagcagggca

SEQ ID NO: 77 - DNA, 120 bp, Homo sapiens
gggcatcttttgaatctacccttcaagtattaagagacagactgtgagcctagcagggcagatcttgtccaccgtgtgtcttcttctgcacgagactttgaggctgtcagagcgcttttt

SEQ ID NO: 78 - DNA, 120 bp, Homo sapiens
tccccggcttaagcagctgctccgctgaccttaaagacatcctgagcgaggccagcaccatgcaactccttcagcaacagcagcaggaagcagtatccgaaggcagcagcagcgggagag

SEQ ID NO: 79 - DNA, 120 bp, Homo sapiens
tggcgagcctgcatggcgcgggtgcagcgggacccggttctgggtcaccctcagccgccgcttcctcatcctggcacactctcttcacagccgaagaaggccagttgtatggaccgtgtg

SEQ ID NO: 80 - DNA, 120 bp, Homo sapiens
ctttcaagggaggttacaccaaagggctagaaggcgagagcctaggctgctctggcagcgctgcagcagggagctccgggacacttgaactgccgtctaccctgtctctctacaagtccg

SEQ ID NO: 81 - DNA, 120 bp, Homo sapiens
ccatttctgacaacgccaaggagttgtgtaaggcagtgtcggtgtccatgggcctgggtgtggaggcgttggagcatctgagtccaggggaacagcttcggggggattgcatgtacgccc

SEQ ID NO: 82 - DNA, 120 bp, Homo sapiens
ctgcagcagggagctccgggacacttgaactgccgtctaccctgtctctctacaagtccggagcactggacgaggcagctgcgtaccagagtcgcgactactacaactttccactggctc

SEQ ID NO: 83 - DNA, 120 bp, Homo sapiens
acccgctggactacggcagcgcctgggcggctgcggcggcgcagtgccgctatggggacctggcgagcctgcatggcgcgggtgcagcgggacccggttctgggtcaccctcagccgccg

SEQ ID NO: 84 - DNA, 120 bp, Homo sapiens
aaggttctctgctagacgacagcgcaggcaagagcactgaagatactgctgagtattcccctttcaagggaggttacaccaaagggctagaaggcgagagcctaggctgctctggcagcg

SEQ ID NO: 85 - DNA, 120 bp, Homo sapiens
cacttttgggagttccacccgctgtgcgtcccactccttgtgccccattggccgaatgcaaaggttctctgctagacgacagcgcaggcaagagcactgaagatactgctgagtattccc

SEQ ID NO: 86 - DNA, 120 bp, Homo sapiens
cacctccggacgaggatgactcagctgccccatccacgttgtccctgctgggccccactttccccggcttaagcagctgctccgctgaccttaaagacatcctgagcgaggccagcacca

SEQ ID NO: 87 - DNA, 120 bp, Homo sapiens
agggtgaggatggttctccccaagcccatcgtagaggccccacaggctacctggtcctggatgaggaacagcaaccttcacagccgcagtcggccctggagtgccaccccgagagaggtt

SEQ ID NO: 88 - DNA, 120 bp, Homo sapiens
tggaggcgttggagcatctgagtccaggggaacagcttcggggggattgcatgtacgccccacttttgggagttccacccgctgtgcgtcccactccttgtgccccattggccgaatgca

SEQ ID NO: 89 - DNA, 120 bp, Homo sapiens
tgcaactccttcagcaacagcagcaggaagcagtatccgaaggcagcagcagcgggagagcgagggaggcctcgggggctcccacttcctccaaggacaattacttagggggcacttcga

SEQ ID NO: 90 - DNA, 120 bp, Homo sapiens
cgagggaggcctcgggggctcccacttcctccaaggacaattacttagggggcacttcgaccatttctgacaacgccaaggagttgtgtaaggcagtgtcggtgtccatgggcctgggtg

SEQ ID NO: 91 - DNA, 120 bp, Homo sapiens
atgaggaacagcaaccttcacagccgcagtcggccctggagtgccaccccgagagaggttgcgtcccagagcctggagccgccgtggccgccagcaaggggctgccgcagcagctgccag

SEQ ID NO: 92 - DNA, 120 bp, Homo sapiens
tggccggaccgccgccccctccgccgcctccccatccccacgctcgcatcaagctggagaacccgctggactacggcagcgcctgggcggctgcggcggcgcagtgccgctatggggacc

SEQ ID NO: 93 - DNA, 120 bp, Homo sapiens
gcgtcccagagcctggagccgccgtggccgccagcaaggggctgccgcagcagctgccagcacctccggacgaggatgactcagctgccccatccacgttgtccctgctgggccccactt

SEQ ID NO: 94 - DNA, 120 bp, Homo sapiens
gagcactggacgaggcagctgcgtaccagagtcgcgactactacaactttccactggctctggccggaccgccgccccctccgccgcctccccatccccacgctcgcatcaagctggaga

SEQ ID NO: 95 - DNA, 120 bp, Homo sapiens
gcttccaattgcagggtcattttggggtgctttcttgcctgtactaattttatctcatcaagcttccattctttgagctgtaaactttgaaataatatactggatttgctggtacgttta

SEQ ID NO: 96 - DNA, 120 bp, Homo sapiens
ctatcttacaagcaacagtttgtcttgtaaagcagaattttcctttgaaaccaagacagattatttctgcccataggcttcaggaaccaatattttggcaagaagcatcttttctttgtg

SEQ ID NO: 97 - DNA, 120 bp, Homo sapiens
gtgattgctgcactgaatatgaggagtctagttaaagggacaactggtgttcctgtcttgtgagttgacgaagactttccatttctaggatatagaaaatccttaagccggtttattgaa

SEQ ID NO: 98 - DNA, 120 bp, Homo sapiens
agagtaggcagaggggcaactttcttggtaaagacttcacaggatttgcactcacagtttctcaacgttggttgactatgttgaaagtagttgcttgggtcggttttctcttgtaaagtg

SEQ ID NO: 99 - DNA, 120 bp, Homo sapiens
agccacactgggtggcaccccattcggaagtatacacaggaagtagccctcttgcttgttcacagctcaagtcagccaaagattaacactggtgagagatattttcaaagaagtttgcag

SEQ ID NO: 100 - DNA, 120 bp, Homo sapiens
gcgaggcgggagctgtagccccctacggctacactcggccccctcaggggctggcgggccaggaaagcgacttcaccgcacctgatgtgtggtaccctggcggcatggtgagcagagtgc

SEQ ID NO: 101 - DNA, 120 bp, Homo sapiens
cacaacttacttacttaacagggaaaaaactgatggttccacatatttgctaaaaaatgtgtgccttcaaagacaaaaccaaaatttttagggaataactatagagagcaaaagttactc

SEQ ID NO: 102 - DNA, 120 bp, Homo sapiens
ctaagagagtgcatgccagaattagagtttggggtttagagaaattatccagatgccaaaagaacattttaatttttctcttggtaatttgttctggtctccatagtaggtagtatttta

SEQ ID NO: 103 - DNA, 120 bp, Homo sapiens
gaaagtggtctctgggtgctgaggtctgctgtgtgaaagggtgaacttctttctcctgaagcaactggggacttgctccagggctggaggtcagtagagataatccaaaccgtcatgttt

SEQ ID NO: 104 - DNA, 120 bp, Homo sapiens
gcaactggggacttgctccagggctggaggtcagtagagataatccaaaccgtcatgtttagagtaggcagaggggcaactttcttggtaaagacttcacaggatttgcactcacagttt

SEQ ID NO: 105 - DNA, 120 bp, Homo sapiens
cagagaaggaaaaggaaatctctccctaccccccacctccaccattttctctttgtctgcagcttcctcaagtgctgcctgtccccgattttcttttattccactcctttcatgtttttg

SEQ ID NO: 106 - DNA, 120 bp, Homo sapiens
gacggtggagcccctcttctactgtaaacttttcttgggggaaaatgtctaaggtgcattttgacctgccatgatactaaacccagacactggaaccttccatcttctgcatgcctcccc

SEQ ID NO: 107 - DNA, 120 bp, Homo sapiens
cttactttctctttttttccctcattttgtagggatacaatttggtgaaaggcaagagatttcttaagccaaagcaagagtgtcttccctctctgtgttgcatgcattatgtgccatgtt

SEQ ID NO: 108 - DNA, 120 bp, Homo sapiens
gtcagcaaataggtggtgagttctgtctggatcccaacaatcaacacctgaggaccaaatagccacactgggtggcaccccattcggaagtatacacaggaagtagccctcttgcttgtt

SEQ ID NO: 109 - DNA, 120 bp, Homo sapiens
aggaaagcgacttcaccgcacctgatgtgtggtaccctggcggcatggtgagcagagtgccctatcccagtcccacttgtgtcaaaagcgaaatgggcccctggatggatagctactccg

SEQ ID NO: 110 - DNA, 120 bp, Homo sapiens
cctatcccagtcccacttgtgtcaaaagcgaaatgggcccctggatggatagctactccggaccttacggggacatgcggtaagtttttccttccagaaatgtcgcctttcggcccaggg

SEQ ID NO: 111 - DNA, 120 bp, Homo sapiens
tgagctaaaaatctcaaaattgggcaggcttccaatgacctgttgggtccctccctttaccattcatgtgtgtgtttatgtacataattttgtggaggggtttttttaaaccttagtaac

SEQ ID NO: 112 - DNA, 120 bp, Homo sapiens
cagagtcactctgtgttctggggtatctagcggctcctacctgcgcgaacactcagattgcccctgggagagctcagcagggtaaacctagagctctcccgtggactcccggcctgccag

SEQ ID NO: 113 - DNA, 120 bp, Homo sapiens
gaccttacggggacatgcggtaagtttttccttccagaaatgtcgcctttcggcccagggcagagtcactctgtgttctggggtatctagcggctcctacctgcgcgaacactcagattg

SEQ ID NO: 114 - DNA, 120 bp, Homo sapiens
ccatcaagtagacaacgagcttggtgattttatttcaggtcttaatgaaaaaagcttctttatgaggaaggttatcatatcttggtgcctccttgacagtccgcttaaattaatgacata

SEQ ID NO: 115 - DNA, 120 bp, Homo sapiens
ctcaacgttggttgactatgttgaaagtagttgcttgggtcggttttctcttgtaaagtgtttattttctctgtggattataacagatccacagccccctacttcaggtttgcatcagat

SEQ ID NO: 116 - DNA, 120 bp, Homo sapiens
ccagattcttcaactcccccaaccgccccaaattctcactacctcctggtactcgaggtcccaaacagaaatcctattgcacgggccaccttcagagataaagctcccaagccctccact

SEQ ID NO: 117 - DNA, 120 bp, Homo sapiens
catgctcctaaagaatttcttttttaaaaaaaatctgtagagtagtagattagattaaccccagtatctctcccttaagactagatgacatgaggggattgcaaaatgaatagctgggtt

SEQ ID NO: 118 - DNA, 120 bp, Homo sapiens
ttcattttataagaacaaatttacttggtagggttaattttttttctagggctgtcactagacggtggagcccctcttctactgtaaacttttcttgggggaaaatgtctaaggtgcatt

SEQ ID NO: 119 - DNA, 120 bp, Homo sapiens
tttaatgtttgacttacaagattttcagagggttcatttgatattgtcaaagtcttttccagttaatttagactcttcatttttgtaatgggtttatgctatgggacaaaaaaagtattc

SEQ ID NO: 120 - DNA, 120 bp, Homo sapiens
agcttccattctttgagctgtaaactttgaaataatatactggatttgctggtacgtttaattttctttgttaagtgttttcattcccatagtaatttttcatctagtgtacatatatgc

SEQ ID NO: 121 - DNA, 120 bp, Homo sapiens
gcaaaggtggtctcttttacttaatttagcatgtggtttgaacagaaggaaaaataaaaagtgatggggcttgtgtgcaaccctgatgatattttatggagctgtctgtcttctctctga

SEQ ID NO: 122 - DNA, 120 bp, Homo sapiens
cttcttacaaggtagatagcacagtgttagtaaagaaagaaagaggagggtaggatttcatattatttcgtgggctgttgaagaaacagcttcttaccaggctttacattccattaggtt

SEQ ID NO: 123 - DNA, 120 bp, Homo sapiens
caagctacttggtgggattatgtgaactggagttagaaatgtggacaattttattatgattatttttaatggtgatatcaagatcaccagtttcattcggaaccttgcataagcagggag

SEQ ID NO: 124 - DNA, 120 bp, Homo sapiens
tcttatattccagcaagcagcacaataatatgacaaaaatttattcttgggagttgggttctaagagagtgcatgccagaattagagtttggggtttagagaaattatccagatgccaaa

SEQ ID NO: 125 - DNA, 120 bp, Homo sapiens
ctataaagaggagaatattcttttaatgtacaatttaattaggcttgactctgacttacaaaactgttggaaaacatttttttgtaaagcatttcctgctatttcagtgtgctccaaaat

SEQ ID NO: 126 - DNA, 120 bp, Homo sapiens
gtagtgctttgatattgacaagtcttgctccctttctctattagatttttcaaaataaggcattttattaattcctctttccttctcctctctcctctcagttatcaagcatttttatga

SEQ ID NO: 127 - DNA, 120 bp, Homo sapiens
gatcaaacaggactacaactttgttaattgaccactggctcccttggcaaaagtagggcttcttatattccagcaagcagcacaataatatgacaaaaatttattcttgggagttgggtt

SEQ ID NO: 128 - DNA, 120 bp, Homo sapiens
ttatttctgcccataggcttcaggaaccaatattttggcaagaagcatcttttctttgtggtcagcaaataggtggtgagttctgtctggatcccaacaatcaacacctgaggaccaaat

SEQ ID NO: 129 - DNA, 120 bp, Homo sapiens
ttctttgtttctatggctgttttcttttacctgtgacttctccgaaatttctttgttagccttaacatctttgtttggggacttaaatccagcaatttgccttctttcactgatgctttc

SEQ ID NO: 130 - DNA, 120 bp, Homo sapiens
ttgacctgccatgatactaaacccagacactggaaccttccatcttctgcatgcctcccccacaacttacttacttaacagggaaaaaactgatggttccacatatttgctaaaaaatgt

SEQ ID NO: 131 - DNA, 120 bp, Homo sapiens
ccaaacagaaatcctattgcacgggccaccttcagagataaagctcccaagccctccactcttcctttcctcctgtcctcaaagtctgagaacctcaacaggaatttgggcaatttctcc

SEQ ID NO: 132 - DNA, 120 bp, Homo sapiens
tatgaggaaggttatcatatcttggtgcctccttgacagtccgcttaaattaatgacataaactaatgagaatttagcagttcctgcagaaagtacaagtttattttttttttctggttt

SEQ ID NO: 133 - DNA, 120 bp, Homo sapiens
tatttttaatggtgatatcaagatcaccagtttcattcggaaccttgcataagcagggagcagaatgcggactgggtgtggcaaagcaagggcttattttatagccaaacctgaaatcac

SEQ ID NO: 134 - DNA, 120 bp, Homo sapiens
cttaacatctttgtttggggacttaaatccagcaatttgccttctttcactgatgctttccttcttacaaggtagatagcacagtgttagtaaagaaagaaagaggagggtaggatttca

SEQ ID NO: 135 - DNA, 120 bp, Homo sapiens
acattgaaatacagactcttctttccacttctcagggtatttttcttattacacctgtggcatgctcctaaagaatttcttttttaaaaaaaatctgtagagtagtagattagattaacc

SEQ ID NO: 136 - DNA, 120 bp, Homo sapiens
agttaatttagactcttcatttttgtaatgggtttatgctatgggacaaaaaaagtattcttcattttataagaacaaatttacttggtagggttaattttttttctagggctgtcacta

SEQ ID NO: 137 - DNA, 120 bp, Homo sapiens
gtgatggggcttgtgtgcaaccctgatgatattttatggagctgtctgtcttctctctgagatcaaacaggactacaactttgttaattgaccactggctcccttggcaaaagtagggct

SEQ ID NO: 138 - DNA, 120 bp, Homo sapiens
aattaatcaatttaatcagaatgcaatcaattccaatacaaaagttagtattttctttctttttattgaaaattaatttaatcagaatacaatcaattccaatccaaaagttgatatttt

SEQ ID NO: 139 - DNA, 120 bp, Homo sapiens
aaactgttggaaaacatttttttgtaaagcatttcctgctatttcagtgtgctccaaaatctccactggggagggtggagtgaggttttttattatattcctttatttttaggacatgtt

SEQ ID NO: 140 - DNA, 120 bp, Homo sapiens
cagaatgcggactgggtgtggcaaagcaagggcttattttatagccaaacctgaaatcacaactctgaaaaataaaaaaaaaaaaaaccaaacaaaaaaatcaagttttgtgagcttggt

SEQ ID NO: 141 - DNA, 120 bp, Homo sapiens
tgagccgtaatcttaccccaaagttttaattagcatatgagaaaagtggcaggcaattgcatcgtgcttattaaaaattattcctcaccgcagttgttgagcttcttggagaccatgctg

SEQ ID NO: 142 - DNA, 120 bp, Homo sapiens
ctccactggggagggtggagtgaggttttttattatattcctttatttttaggacatgtttgcattttagaatatgtgcagttagctctaacaaattgagtaagaactcttaatgaccta

SEQ ID NO: 143 - DNA, 120 bp, Homo sapiens
cacagctcaagtcagccaaagattaacactggtgagagatattttcaaagaagtttgcaggcttccaattgcagggtcattttggggtgctttcttgcctgtactaattttatctcatca

SEQ ID NO: 144 - DNA, 120 bp, Homo sapiens
atttaaaacaaaaattctttggtctccttatgcgtatatgcactgcggcttgtacacgtacaagctacttggtgggattatgtgaactggagttagaaatgtggacaattttattatgat

SEQ ID NO: 145 - DNA, 120 bp, Homo sapiens
ttttattgaaaattaatttaatcagaatacaatcaattccaatccaaaagttgatattttcttactttctctttttttccctcattttgtagggatacaatttggtgaaaggcaagagat

SEQ ID NO: 146 - DNA, 120 bp, Homo sapiens
gtgccttcaaagacaaaaccaaaatttttagggaataactatagagagcaaaagttactcccatcaagtagacaacgagcttggtgattttatttcaggtcttaatgaaaaaagcttctt

SEQ ID NO: 147 - DNA, 120 bp, Homo sapiens
aggtttaacctgagctctcctaatttctgctgcgtgccctgggtgctgattcctgccctcccagattcttcaactcccccaaccgccccaaattctcactacctcctggtactcgaggtc

SEQ ID NO: 148 - DNA, 120 bp, Homo sapiens
tattatttcgtgggctgttgaagaaacagcttcttaccaggctttacattccattaggtttttaatgtttgacttacaagattttcagagggttcatttgatattgtcaaagtcttttcc

SEQ ID NO: 149 - DNA, 120 bp, Homo sapiens
cattttattaattcctctttccttctcctctctcctctcagttatcaagcatttttatgactatcttacaagcaacagtttgtcttgtaaagcagaattttcctttgaaaccaagacaga

SEQ ID NO: 150 - DNA, 120 bp, Homo sapiens
cttcctttcctcctgtcctcaaagtctgagaacctcaacaggaatttgggcaatttctcctcttcaggtctgttaggatttcactttcagcctgcgcagattagagtcaaaaagaccggc

SEQ ID NO: 151 - DNA, 120 bp, Homo sapiens
attttctttgttaagtgttttcattcccatagtaatttttcatctagtgtacatatatgcatttaaaacaaaaattctttggtctccttatgcgtatatgcactgcggcttgtacacgta

SEQ ID NO: 152 - DNA, 120 bp, Homo sapiens
attggggaattaactcctcaggtaggccaggtgctgatgtccctgtggacttttgtcttattctttgtttctatggctgttttcttttacctgtgacttctccgaaatttctttgttagc

SEQ ID NO: 153 - DNA, 120 bp, Homo sapiens
tttattttctctgtggattataacagatccacagccccctacttcaggtttgcatcagatctataaagaggagaatattcttttaatgtacaatttaattaggcttgactctgacttaca

SEQ ID NO: 154 - DNA, 120 bp, Homo sapiens
aactctgaaaaataaaaaaaaaaaaaaccaaacaaaaaaatcaagttttgtgagcttggtcagagaaggaaaaggaaatctctccctaccccccacctccaccattttctctttgtctgc

SEQ ID NO: 155 - DNA, 120 bp, Homo sapiens
tgcattttagaatatgtgcagttagctctaacaaattgagtaagaactcttaatgacctatgagccgtaatcttaccccaaagttttaattagcatatgagaaaagtggcaggcaattgc

SEQ ID NO: 156 - DNA, 120 bp, Homo sapiens
atcgtgcttattaaaaattattcctcaccgcagttgttgagcttcttggagaccatgctgaagattttctcccccagcaaattaagatattagtttatctgctgagggaggacagactga

SEQ ID NO: 157 - DNA, 120 bp, Homo sapiens
ttcttaagccaaagcaagagtgtcttccctctctgtgttgcatgcattatgtgccatgtttgagctaaaaatctcaaaattgggcaggcttccaatgacctgttgggtccctccctttac

SEQ ID NO: 158 - DNA, 120 bp, Homo sapiens
ccaatagcttctcagcgggtatcctccagagaggtaaagtgaaattctcggttagggaaagaaagtggtctctgggtgctgaggtctgctgtgtgaaagggtgaacttctttctcctgaa

SEQ ID NO: 159 - DNA, 120 bp, Homo sapiens
aagattttctcccccagcaaattaagatattagtttatctgctgagggaggacagactgaattggggaattaactcctcaggtaggccaggtgctgatgtccctgtggacttttgtctta

SEQ ID NO: 160 - DNA, 120 bp, Homo sapiens
tcttcaggtctgttaggatttcactttcagcctgcgcagattagagtcaaaaagaccggcccaatagcttctcagcgggtatcctccagagaggtaaagtgaaattctcggttagggaaa

SEQ ID NO: 161 - DNA, 120 bp, Homo sapiens
cattcatgtgtgtgtttatgtacataattttgtggaggggtttttttaaaccttagtaacatctgcactcactctgtgttcttatacatttacagtgtttctgctgagaggagggaagat

SEQ ID NO: 162 - DNA, 120 bp, Homo sapiens
agcttcctcaagtgctgcctgtccccgattttcttttattccactcctttcatgtttttgacattgaaatacagactcttctttccacttctcagggtatttttcttattacacctgtgg

SEQ ID NO: 163 - DNA, 120 bp, Homo sapiens
aactaatgagaatttagcagttcctgcagaaagtacaagtttattttttttttctggtttgtgattgctgcactgaatatgaggagtctagttaaagggacaactggtgttcctgtcttg

SEQ ID NO: 164 - DNA, 120 bp, Homo sapiens
tgagttgacgaagactttccatttctaggatatagaaaatccttaagccggtttattgaaaattaatcaatttaatcagaatgcaatcaattccaatacaaaagttagtattttctttct

SEQ ID NO: 165 - DNA, 120 bp, Homo sapiens
agaacattttaatttttctcttggtaatttgttctggtctccatagtaggtagtattttagtagtgctttgatattgacaagtcttgctccctttctctattagatttttcaaaataagg

SEQ ID NO: 166 - DNA, 120 bp, Homo sapiens
atctgcactcactctgtgttcttatacatttacagtgtttctgctgagaggagggaagatgcaaaggtggtctcttttacttaatttagcatgtggtttgaacagaaggaaaaataaaaa

SEQ ID NO: 167 - DNA, 120 bp, Homo sapiens
cccctgggagagctcagcagggtaaacctagagctctcccgtggactcccggcctgccagaggtttaacctgagctctcctaatttctgctgcgtgccctgggtgctgattcctgccctc

SEQ ID NO: 168 - DNA, 120 bp, Homo sapiens
gctaggtaacaaagtagaaagttagattttctatgatatttgtttaccacgtaggggaacctctctagagcaatactcccaagctttttcttcttgaaatttcccacctgacagataata

SEQ ID NO: 169 - DNA, 120 bp, Homo sapiens
gatgttaaattcagagaaagttaaccttatcttaaacacaaagttgacttttaaacaaaattgcttataaagttctgtacagttaccagcattggttgccctttgtcgtacggaagagaa

SEQ ID NO: 170 - DNA, 120 bp, Homo sapiens
agatattaaagaaattattctatctccgacttcccctatcagcattccatcaagttctgggatgttaaattcagagaaagttaaccttatcttaaacacaaagttgacttttaaacaaaa

SEQ ID NO: 171 - DNA, 120 bp, Homo sapiens
agacctcagtagctggatcacaagcagtacccaatatgcatatgagggtgcgtgctgcaagtgtccggctgggctaatctgcttaagcttcataaaaattaatcatttgaaaacaaagaa

SEQ ID NO: 172 - DNA, 120 bp, Homo sapiens
gtgtccggctgggctaatctgcttaagcttcataaaaattaatcatttgaaaacaaagaaagatattaaagaaattattctatctccgacttcccctatcagcattccatcaagttctgg

SEQ ID NO: 173 - DNA, 120 bp, Homo sapiens
ctgccaacctgcaggccacaccaggattgagggcagctcatctcgataaatttatagcattaaagtgctgggtcatttgagaatgttgtcaatttaggttacttagtacctaagttttat

SEQ ID NO: 174 - DNA, 120 bp, Homo sapiens
tgaattattgcttgcacactcatgggtgatgctactccctctctctcatggcaattcttgctgccaacctgcaggccacaccaggattgagggcagctcatctcgataaatttatagcat

SEQ ID NO: 175 - DNA, 120 bp, Homo sapiens
ttatgaaatctcatatttacatagcattcttccaaaaaaagagacggtgttttccagtttattcactgcattcgtgtaagtgtgagtaggccaggaggggtgcttagtgattaccctttt

SEQ ID NO: 176 - DNA, 120 bp, Homo sapiens
ctctctagagcaatactcccaagctttttcttcttgaaatttcccacctgacagataatactttagattgttgctcttaaggacttctctcagtagctgctacatagagatgattgtccg

SEQ ID NO: 177 - DNA, 120 bp, Homo sapiens
attcactgcattcgtgtaagtgtgagtaggccaggaggggtgcttagtgattacccttttgctaggtaacaaagtagaaagttagattttctatgatatttgtttaccacgtaggggaac

SEQ ID NO: 178 - DNA, 120 bp, Homo sapiens
gctactttctctcagagcctgagagacagctctgagacacttcccaggtctgttcggttcagacctcagtagctggatcacaagcagtacccaatatgcatatgagggtgcgtgctgcaa

SEQ ID NO: 179 - DNA, 120 bp, Homo sapiens
ttgcttataaagttctgtacagttaccagcattggttgccctttgtcgtacggaagagaattatgaaatctcatatttacatagcattcttccaaaaaaagagacggtgttttccagttt

SEQ ID NO: 180 - DNA, 120 bp, Homo sapiens
ctttagattgttgctcttaaggacttctctcagtagctgctacatagagatgattgtccgtgaattattgcttgcacactcatgggtgatgctactccctctctctcatggcaattcttg

SEQ ID NO: 181 - DNA, 120 bp, Homo sapiens
tataaattcgaactggtaatcccatcccctttcgggatgaataggagagtgtttttaaatgttcatctctttagagaacagcaggaaagaagcctagtaaggtttgggtagtttataatc

SEQ ID NO: 182 - DNA, 120 bp, Homo sapiens
gctaatcagctacaactgtaatgtcctgataattgtgaattaactgcagggcacccagcaaaaggtttagttataatctaatagctgtctgtagagattagcctaataaagggatttttt

SEQ ID NO: 183 - DNA, 120 bp, Homo sapiens
ccttttttagaatttggatttgggaactattagcaaggcagtgagtaataataataatttctatatagaaaactaacatgtagaggtgacaaatgaaatcactagctatattaggcttat

SEQ ID NO: 184 - DNA, 120 bp, Homo sapiens
ttacttcatttcatcctgtatgaaggctgcatggggacattcttctcagttttactcagctataaattcgaactggtaatcccatcccctttcgggatgaataggagagtgtttttaaat

SEQ ID NO: 185 - DNA, 120 bp, Homo sapiens
aaattcatctatacacagactgaactttgtcttcattaacactctaggctaagggtcatagctaatcagctacaactgtaatgtcctgataattgtgaattaactgcagggcacccagca

SEQ ID NO: 186 - DNA, 120 bp, Homo sapiens
gttcatctctttagagaacagcaggaaagaagcctagtaaggtttgggtagtttataatcccttttttagaatttggatttgggaactattagcaaggcagtgagtaataataataattt

SEQ ID NO: 187 - DNA, 120 bp, Homo sapiens
gtttaggttatcgtaagcagctaaaatcataattttatgtttttatatgttgtcctttggacaaagtaaattccagtactccttctgatgtgcatttctagatggggaaaggattcattt

SEQ ID NO: 188 - DNA, 120 bp, Homo sapiens
ctatatagaaaactaacatgtagaggtgacaaatgaaatcactagctatattaggcttatgtttaggttatcgtaagcagctaaaatcataattttatgtttttatatgttgtcctttgg

SEQ ID NO: 189 - DNA, 120 bp, Homo sapiens
actctcatataatttaagcttctttttagggatgtactccatagccatgaagcaaagataaaattcatctatacacagactgaactttgtcttcattaacactctaggctaagggtcata

SEQ ID NO: 190 - DNA, 120 bp, Homo sapiens
ttgaatgcttctgggacttatggggaagagggcttctgctgctgcactgaaagttaaagcttacttcatttcatcctgtatgaaggctgcatggggacattcttctcagttttactcagc

SEQ ID NO: 191 - DNA, 120 bp, Homo sapiens
acaaagtaaattccagtactccttctgatgtgcatttctagatggggaaaggattcatttactctcatataatttaagcttctttttagggatgtactccatagccatgaagcaaagata

SEQ ID NO: 192 - DNA, 120 bp, Homo sapiens
caataatatgagtaataaattaaaattgaaggtgattaatggctctgaatttgacataagagttgttttcctgccttctaagtttccattgatcctgatgaattgcacaaaccaaacaat

SEQ ID NO: 193 - DNA, 120 bp, Homo sapiens
cattctgaaatggcctaccacctaacatgggctctgttctctgcaggttgagtaggttccttgcttgtggaactgtagtcccgctatttggccgctagggggactgcaagtgccccgtgg

SEQ ID NO: 194 - DNA, 120 bp, Homo sapiens
atcctggattgtacggtatggatttcttaaaatgtagatattttaaaaaaaaagaggaatgaatcaatagaggctgaagtggtcagcaatgttacctgtggctgcttttaatccttcgtg

SEQ ID NO: 195 - DNA, 120 bp, Homo sapiens
tggctatatcattgcacttggtgggtacatgtttatgatgtctttatctgaacaagtcagcaataatatgagtaataaattaaaattgaaggtgattaatggctctgaatttgacataag

SEQ ID NO: 196 - DNA, 120 bp, Homo sapiens
atcgcaggataacttgaaccagaactgcctagcaccagacaataaataagctactatggtacttactgtttcatttgggatgttgtttctcgaagtggcaagcattttttagtaatattt

SEQ ID NO: 197 - DNA, 120 bp, Homo sapiens
aaataacagaagttagtaccactggaaagaatgaactggaggaatgggttgaaatctatttctgcttattcaatagtgcaccccagtcaagttagttgccaatttcttcttcagtttctt

SEQ ID NO: 198 - DNA, 120 bp, Homo sapiens
agttgttttcctgccttctaagtttccattgatcctgatgaattgcacaaaccaaacaattcggggagtaagggggcacatgatgatcttataagagctttgctgtattagacaacgtaa

SEQ ID NO: 199 - DNA, 120 bp, Homo sapiens
ttgcttgtggaactgtagtcccgctatttggccgctagggggactgcaagtgccccgtggcaggatttccctgggaatggtgagcctccattgatggtttcaacacacagccaaggccct

SEQ ID NO: 200 - DNA, 120 bp, Homo sapiens
accaaacaaccaaaaaccaaacagaaatgcagtatcatcatgccatgatgcctgtatgagatcctggattgtacggtatggatttcttaaaatgtagatattttaaaaaaaaagaggaat

SEQ ID NO: 201 - DNA, 120 bp, Homo sapiens
acttactgtttcatttgggatgttgtttctcgaagtggcaagcattttttagtaatattttgactttttaatacctttctttgcatatggagcagaaaacagtgacactggatatattca

SEQ ID NO: 202 - DNA, 120 bp, Homo sapiens
tctgcttattcaatagtgcaccccagtcaagttagttgccaatttcttcttcagtttctttggctatatcattgcacttggtgggtacatgtttatgatgtctttatctgaacaagtcag

SEQ ID NO: 203 - DNA, 120 bp, Homo sapiens
tcggggagtaagggggcacatgatgatcttataagagctttgctgtattagacaacgtaacattctgaaatggcctaccacctaacatgggctctgttctctgcaggttgagtaggttcc

SEQ ID NO: 204 - DNA, 120 bp, Homo sapiens
caggatttccctgggaatggtgagcctccattgatggtttcaacacacagccaaggccctatcgcaggataacttgaaccagaactgcctagcaccagacaataaataagctactatggt

SEQ ID NO: 205 - DNA, 120 bp, Homo sapiens
gaatcaatagaggctgaagtggtcagcaatgttacctgtggctgcttttaatccttcgtggaagtaagtaggagcatgtctaaactcaagcaatagattaaagatcttgatgtatatttt

SEQ ID NO: 206 - DNA, 120 bp, Homo sapiens
gaagtaagtaggagcatgtctaaactcaagcaatagattaaagatcttgatgtatattttaaataacagaagttagtaccactggaaagaatgaactggaggaatgggttgaaatctatt

SEQ ID NO: 207 - DNA, 120 bp, Homo sapiens
gagaggcaaaagaaccacaagtggtatcaatactagaaatttatgaatttcttaaggcttctaggtttgttacccatccaccagactgatggatttggttgtgtgagagttctgggtgcc

SEQ ID NO: 208 - DNA, 120 bp, Homo sapiens
aatcacactgaaatattattatttttactgaaccacataccaaaatatttttcctgtaaaaacacagtaagtgaacttttaaaggcaattgagcttttaacaaagctagaatctacagag

SEQ ID NO: 209 - DNA, 120 bp, Homo sapiens
aggtaattaaactaaatagtgaataaaactgggaaactatacaaattggttgctctccccaatcacactgaaatattattatttttactgaaccacataccaaaatatttttcctgtaaa

SEQ ID NO: 210 - DNA, 120 bp, Homo sapiens
gggcaagagtgagttttttgttattgttttttgtttgtttctttgcccaaacctaaaaccaggtaattaaactaaatagtgaataaaactgggaaactatacaaattggttgctctcccc

SEQ ID NO: 211 - DNA, 120 bp, Homo sapiens
cctagtcagagcctgttttaagggttagtcgtatgttgttttcttgaaaaaagttacattggaaaagtgaaaattctttggtccatactgagaacaaagaattatacataatcatatata

SEQ ID NO: 212 - DNA, 120 bp, Homo sapiens
agagatcccaaggaggttgatctccgactgctacaaacctgggcaattcaatgcctgcttaaataggagagttaagataagaaaaataaaattgccaatttttacagtcagacattgttt

SEQ ID NO: 213 - DNA, 120 bp, Homo sapiens
agttgatgactcatgatagggattggaaaacaggactacaggaattattgaaaagggcctagagatcccaaggaggttgatctccgactgctacaaacctgggcaattcaatgcctgctt

SEQ ID NO: 214 - DNA, 120 bp, Homo sapiens
taggtgggtggcctagaggttaaagtagaggcagagtgatggaaaggggtggttagaagaagttgatgactcatgatagggattggaaaacaggactacaggaattattgaaaagggcct

SEQ ID NO: 215 - DNA, 120 bp, Homo sapiens
ggtgacgtaggtgacttcattatgctctgcccctattatagtccactgatcctcaccaaataggtgggtggcctagaggttaaagtagaggcagagtgatggaaaggggtggttagaaga

SEQ ID NO: 216 - DNA, 120 bp, Homo sapiens
ctactgtgcaataccaggagcttctcagattacccttcacctttaccaacccaaatgactggtgacgtaggtgacttcattatgctctgcccctattatagtccactgatcctcaccaaa

SEQ ID NO: 217 - DNA, 120 bp, Homo sapiens
tatggaatgaggtaattttcttataacagaaagtttttaaaatgcaaaaacattgtgcctgaacttcaaacactgaacaactcatatccttaatatgcaccagtttcttttaagcactct

SEQ ID NO: 218 - DNA, 120 bp, Homo sapiens
tgtctctccaggttttcttttaaagaaggtagtcttgctaaatgataactatttcagcatttatttgaaaatgggcagtgcaggagagaaagaatttttccaagcttgtcacattgggcc

SEQ ID NO: 219 - DNA, 120 bp, Homo sapiens
acctctctgaagcattgtccaacttctaattagatgaggagactgcataaaccaagagttgagagtaaagatggaaacacttgatgtttggtgtttgggtgcagaaaggattccagaaca

SEQ ID NO: 220 - DNA, 120 bp, Homo sapiens
aggattagtgcagtgatcccacgtcctttctctctagctctctctgctactctctaattcctattgtatttgtgccaccagatctttccaaagtttagctccaatcttgtctgtatactg

SEQ ID NO: 221 - DNA, 120 bp, Homo sapiens
tgtggttgtcagataacctttttgttactgtggaaatggaagcaggctactgcaaaaatctgtctctccaggttttcttttaaagaaggtagtcttgctaaatgataactatttcagcat

SEQ ID NO: 222 - DNA, 120 bp, Homo sapiens
tctgggcttcagtgaggtgctagattgactcaaaatatggcaggtcagatgtgggattgagcagggtggactcttctctacccttcccaattcagagttccccatcaaagatgatctcat

SEQ ID NO: 223 - DNA, 120 bp, Homo sapiens
gcagggtggactcttctctacccttcccaattcagagttccccatcaaagatgatctcatagtgtttgaaaaaccaagctgaaggctttgggaattagggtgctgaagggatatgctgtt

SEQ ID NO: 224 - DNA, 120 bp, Homo sapiens
gaccaaaaattttaaccatgctcatactttcatatggtatgtagtttgctttagacattttctgggcttcagtgaggtgctagattgactcaaaatatggcaggtcagatgtgggattga

SEQ ID NO: 225 - DNA, 120 bp, Homo sapiens
tcccaaagccttctcagtcattccttctccccccagttcagattcttaacacctctttccaggattagtgcagtgatcccacgtcctttctctctagctctctctgctactctctaattc

SEQ ID NO: 226 - DNA, 120 bp, Homo sapiens
ttcaccttttaattacctgtctccatcaacaagattggacagagaattgggagagtgagcagagtccatttccttccagagactggacaaaaggaacaaaatgttaggaaaaaatgtcag

SEQ ID NO: 227 - DNA, 120 bp, Homo sapiens
ttatttgaaaatgggcagtgcaggagagaaagaatttttccaagcttgtcacattgggccacctctctgaagcattgtccaacttctaattagatgaggagactgcataaaccaagagtt

SEQ ID NO: 228 - DNA, 120 bp, Homo sapiens
catgtgggatttgtgggatttacactaaataagaagggacacttcccaggactgacaagatgctacctccgtccctctaggccccaatgtgttgtgcaggatcccataggaagtcatgaa

SEQ ID NO: 229 - DNA, 120 bp, Homo sapiens
tgctacctccgtccctctaggccccaatgtgttgtgcaggatcccataggaagtcatgaatgtggttgtcagataacctttttgttactgtggaaatggaagcaggctactgcaaaaatc

SEQ ID NO: 230 - DNA, 120 bp, Homo sapiens
ttttgaataaaaaagaccaaattagactgagatatttcagtcaccaactatctaataatagaccaaaaattttaaccatgctcatactttcatatggtatgtagtttgctttagacattt

SEQ ID NO: 231 - DNA, 120 bp, Homo sapiens
gagagtaaagatggaaacacttgatgtttggtgtttgggtgcagaaaggattccagaacatgttttgggtctctttactctgtccatccctccttccctttcatctttgtttaaaaacca

SEQ ID NO: 232 - DNA, 120 bp, Homo sapiens
ttgggctcccttctaacattgacttgtctccctccattcctcctccgtattgttctgcccttcaccttttaattacctgtctccatcaacaagattggacagagaattgggagagtgagc

SEQ ID NO: 233 - DNA, 120 bp, Homo sapiens
cagttagcaaatgtgtagtctgtttgcaattgttcatctgaaaaatttgtttgatcagccttttgaataaaaaagaccaaattagactgagatatttcagtcaccaactatctaataata

SEQ ID NO: 234 - DNA, 120 bp, Homo sapiens
agagtccatttccttccagagactggacaaaaggaacaaaatgttaggaaaaaatgtcagcatgtgggatttgtgggatttacactaaataagaagggacacttcccaggactgacaaga

SEQ ID NO: 235 - DNA, 120 bp, Homo sapiens
tgttttgggtctctttactctgtccatccctccttccctttcatctttgtttaaaaaccacagttagcaaatgtgtagtctgtttgcaattgttcatctgaaaaatttgtttgatcagcc

SEQ ID NO: 236 - DNA, 120 bp, Homo sapiens
agtgtttgaaaaaccaagctgaaggctttgggaattagggtgctgaagggatatgctgtttcccaaagccttctcagtcattccttctccccccagttcagattcttaacacctctttcc

SEQ ID NO: 237 - DNA, 120 bp, Homo sapiens
cttacaaaactcttaaaacccagccaaaaggatctagtcactgtcactttaaaccatcctcactctcttgttttttgaacatgttatttttcttataatccctttgaccttgaaggctat

SEQ ID NO: 238 - DNA, 120 bp, Homo sapiens
cccaatttcaatactatccattcttctatgacagccccctacaaaatgaatattctcaacctcccaacccaaggagaagtgatctatatgacacaatatggttgaaagaatgttggcttc

SEQ ID NO: 239 - DNA, 120 bp, Homo sapiens
tctggtgttcaagtccttttatgatctgcttatttttccagcctgaattcctggagttcccttacaaaactcttaaaacccagccaaaaggatctagtcactgtcactttaaaccatcct

SEQ ID NO: 240 - DNA, 120 bp, Homo sapiens
cactctcttgttttttgaacatgttatttttcttataatccctttgaccttgaaggctatcccaatttcaatactatccattcttctatgacagccccctacaaaatgaatattctcaac

SEQ ID NO: 241 - DNA, 120 bp, Homo sapiens
catgattacaatttcagcttctccccattggcttatgggttaaagtccaaattatttaaatctggtgttcaagtccttttatgatctgcttatttttccagcctgaattcctggagttcc

SEQ ID NO: 242 - DNA, 120 bp, Homo sapiens
cttttttctttctccatcaatatggtacttagtcccttaaatcagaagtacttgtgttaatgtctgataacgtccttctaaatatacctctaaacatctgtctctctttagggcaaaggt

SEQ ID NO: 243 - DNA, 120 bp, Homo sapiens
tgtcatccccatcagatcagagacctaagcaaatcttgggtcccttgcttactccaagggctttcactcctcgtataggaggagctaaagaaatgtacaagcagcaccacaataggatca

SEQ ID NO: 244 - DNA, 120 bp, Homo sapiens
aaagcctatagatgactccaaaatgcccatttggatgatatgaggcatactttgtgtagttaaggattttaaatacataacagagaggctgaagggccttcgggaaagaagctggggtaa

SEQ ID NO: 245 - DNA, 120 bp, Homo sapiens
gttgatgtgtgcttcaaagtgtgagacagagctagtctgaggagagagggagagtgagaagattcctcttcttggccagaggtcatggtcttccacaaggaacagaatgactcaatgcaa

SEQ ID NO: 246 - DNA, 120 bp, Homo sapiens
attatgggacctctttgagtttggggcccctacatttaaactagtaactccgttgcacatattggcacccttcccccaacaaaattactgggcaggaattttcttgaatccttccgtggc

SEQ ID NO: 247 - DNA, 120 bp, Homo sapiens
ttgcttcggtaatgcaagttattaagttacttccctcagcccagctgaaatctcttattggttgatgtgtgcttcaaagtgtgagacagagctagtctgaggagagagggagagtgagaa

SEQ ID NO: 248 - DNA, 120 bp, Homo sapiens
attggcacccttcccccaacaaaattactgggcaggaattttcttgaatccttccgtggcctggaatgatctcccttctcatccttgtgatccacacagctggcaaatggcaggcagcag

SEQ ID NO: 249 - DNA, 120 bp, Homo sapiens
acatgggcttttccccaggccactgctgcctggcttccccttccacaaagctttgagtctccaaaatgctttggctggaatgtaagcgtgaggtcattgcagataacaggggagcatgat

SEQ ID NO: 250 - DNA, 120 bp, Homo sapiens
gattcctcttcttggccagaggtcatggtcttccacaaggaacagaatgactcaatgcaaattatgggacctctttgagtttggggcccctacatttaaactagtaactccgttgcacat

SEQ ID NO: 251 - DNA, 120 bp, Homo sapiens
aacaaaaacaagcctcttagcatatagggagagaaagagtcacagcagtactgaatttgcttgggaacctaatgttaacaaaggaccttcctctcaacaccccaaacagattaaaacatt

SEQ ID NO: 252 - DNA, 120 bp, Homo sapiens
ttgggaacctaatgttaacaaaggaccttcctctcaacaccccaaacagattaaaacatttttttaacagcaagttgtgtctcggagcagctctttgcttgggtatatttaaagatctgc

SEQ ID NO: 253 - DNA, 120 bp, Homo sapiens
tttttaacagcaagttgtgtctcggagcagctctttgcttgggtatatttaaagatctgctgagtcatttaagagcaggctggcatatcctaagaggcaaggactataccccagtctatg

SEQ ID NO: 254 - DNA, 120 bp, Homo sapiens
tccacttccatctcccacgactctggagagcatctactaagccttcttattctatcaactttgaactcctcagtgtataatagagtaagggtgagagggaaggagcagtcgtaccagtgt

SEQ ID NO: 255 - DNA, 120 bp, Homo sapiens
ggggagtaagttgagaggtgaaatctgtttggctttctcccatggaaacaaacaaggtgatccacttccatctcccacgactctggagagcatctactaagccttcttattctatcaact

SEQ ID NO: 256 - DNA, 120 bp, Homo sapiens
tagtagtagtagtagtccagacaaacagagcttgggaaaacgctagactctggctgacatacatgggcttttccccaggccactgctgcctggcttccccttccacaaagctttgagtct

SEQ ID NO: 257 - DNA, 120 bp, Homo sapiens
ccaaaatgctttggctggaatgtaagcgtgaggtcattgcagataacaggggagcatgatttgcttcggtaatgcaagttattaagttacttccctcagcccagctgaaatctcttattg

SEQ ID NO: 258 - DNA, 120 bp, Homo sapiens
tgagtcatttaagagcaggctggcatatcctaagaggcaaggactataccccagtctatgggggagtaagttgagaggtgaaatctgtttggctttctcccatggaaacaaacaaggtga

SEQ ID NO: 259 - DNA, 120 bp, Homo sapiens
ctggaatgatctcccttctcatccttgtgatccacacagctggcaaatggcaggcagcagaacaaaaacaagcctcttagcatatagggagagaaagagtcacagcagtactgaatttgc

SEQ ID NO: 260 - DNA, 120 bp, Homo sapiens
ccctcttgcattttgtttatcgatggttttcaaaggacttagaggctggctttgttatagttagttggtaagagaaatggtggaggaccggaaaatgggagtggaacgaatgagcatgtt

SEQ ID NO: 261 - DNA, 120 bp, Homo sapiens
gccatctccttgccatcaaaaccatcctcacagacccttctgaaaccacttctaggaagggaaatcacaatggatccatgaaggatgctttctggatgactttaaaagattggtattaag

SEQ ID NO: 262 - DNA, 120 bp, Homo sapiens
gaaatcacaatggatccatgaaggatgctttctggatgactttaaaagattggtattaagatattttatcagtggtagcaacactgacttattcaggcagccatgccccggatctataag

SEQ ID NO: 263 - DNA, 120 bp, Homo sapiens
atattttatcagtggtagcaacactgacttattcaggcagccatgccccggatctataagaaatcaggtaagctaaaagttgcttgagctggcaggagacctagttctcttttttccttt

SEQ ID NO: 264 - DNA, 120 bp, Homo sapiens
ccaattttccccctggttttcatcttgtaaatctgtaatcctaaaaattagcaaaacctagagcttctctttggtcctggcctgtttgaaccctgttccacagaccccaatcttctttct

SEQ ID NO: 265 - DNA, 120 bp, Homo sapiens
gtggagaaattagcagttaccaactgttcattataggtacacattggggtttccttagagccaattttccccctggttttcatcttgtaaatctgtaatcctaaaaattagcaaaaccta

SEQ ID NO: 266 - DNA, 120 bp, Homo sapiens
gctagaaagtgaaataattttttttctactttattctccactgcacttctaaatttattagtggagaaattagcagttaccaactgttcattataggtacacattggggtttccttagag

SEQ ID NO: 267 - DNA, 120 bp, Homo sapiens
tctctagcacctcagactgtcttcccacagtggcacagcctcccactccactttcactgtgccatctccttgccatcaaaaccatcctcacagacccttctgaaaccacttctaggaagg

SEQ ID NO: 268 - DNA, 120 bp, Homo sapiens
tgtttgaggcaactatccttctctttgcccaccgccattttccttcatctacttttcccttctctagcacctcagactgtcttcccacagtggcacagcctcccactccactttcactgt

SEQ ID NO: 269 - DNA, 120 bp, Homo sapiens
gagcttctctttggtcctggcctgtttgaaccctgttccacagaccccaatcttctttcttgtttgaggcaactatccttctctttgcccaccgccattttccttcatctacttttccct

SEQ ID NO: 270 - DNA, 120 bp, Homo sapiens
aaatcaggtaagctaaaagttgcttgagctggcaggagacctagttctcttttttcctttccctcttgcattttgtttatcgatggttttcaaaggacttagaggctggctttgttatag

SEQ ID NO: 271 - DNA, 120 bp, Homo sapiens
ttgtaatggccctgctattggtagctgagagtagcatggaagtgtcaggttgatgggttcatttaattcttttcttttcagtttcagtcacatgcattgttaccatggcatatgacagtt

SEQ ID NO: 272 - DNA, 120 bp, Homo sapiens
atttaattcttttcttttcagtttcagtcacatgcattgttaccatggcatatgacagttgctagaaagtgaaataattttttttctactttattctccactgcacttctaaatttatta

SEQ ID NO: 273 - DNA, 120 bp, Homo sapiens
cactgctcccagccccagaccattaatttttgacagtaagtccaacttttttcaagttcacagctcagatttgctattgaatgaatgagtatatatgtcatttgggaacattctttccaa

SEQ ID NO: 274 - DNA, 120 bp, Homo sapiens
cttttggttgaagatttgttttatcacttgtgaaaattttttttcattcttagcaatgtcagtttagttaaatgagcatttcatttgcgaattcactaattaattattttattcatcaat

SEQ ID NO: 275 - DNA, 120 bp, Homo sapiens
cagctcagatttgctattgaatgaatgagtatatatgtcatttgggaacattctttccaacttttggttgaagatttgttttatcacttgtgaaaattttttttcattcttagcaatgtc

SEQ ID NO: 276 - DNA, 120 bp, Homo sapiens
ttcagcctctgtgtgtgtatttgtgtgtgtgaaggttgagtgtgtgatgatggatggggctgcgagattgttaagtaggatctatggggggccttaaatggtcctggtgagtcccaactt

SEQ ID NO: 277 - DNA, 120 bp, Homo sapiens
tgcgagattgttaagtaggatctatggggggccttaaatggtcctggtgagtcccaactttctggttatgtatttgagtagagtatgggggtgacaaagattgttgtttaagagttgatt

SEQ ID NO: 278 - DNA, 120 bp, Homo sapiens
ttagattttttccaagtaaatggtcagctgacttggagcatcatcattccacttgctttgaaaacctgccacttaaggctccttccagtcataggttaactctttctggtcaagtattac

SEQ ID NO: 279 - DNA, 120 bp, Homo sapiens
tagtaaacatatgaacagcaacttgtgaaacgccattagcaaaatctcaagttatattcttcagtgactatggccatcctaaaaatggggtgtcttttatttggggtaaatgaagatgaa

SEQ ID NO: 280 - DNA, 120 bp, Homo sapiens
tcagtgactatggccatcctaaaaatggggtgtcttttatttggggtaaatgaagatgaagccttatgagaaattgcattttaatctaatcttgtcttgctaagaacagaagtggaatgt

SEQ ID NO: 281 - DNA, 120 bp, Homo sapiens
tctggttatgtatttgagtagagtatgggggtgacaaagattgttgtttaagagttgattttagattttttccaagtaaatggtcagctgacttggagcatcatcattccacttgctttg

SEQ ID NO: 282 - DNA, 120 bp, Homo sapiens
gccttatgagaaattgcattttaatctaatcttgtcttgctaagaacagaagtggaatgtttcagcctctgtgtgtgtatttgtgtgtgtgaaggttgagtgtgtgatgatggatggggc

SEQ ID NO: 283 - DNA, 120 bp, Homo sapiens
ctcttaagtgccttatttaactgtaatatggaaaatcaaagtcacagctaattcaggaaaaatgagtttgggatgtgaatttcctaggcaacttgtcatctcttttttacttccttagct

SEQ ID NO: 284 - DNA, 120 bp, Homo sapiens
tcataaacttacccacaatgttccctgaggactaagagtaatggagggtgatgaggaaaggctttcctcccttcctttccgagagtcctttagccaaatgccacacctcctcctgtttcc

SEQ ID NO: 285 - DNA, 120 bp, Homo sapiens
ctagtctccgtgcagagatggaagtgggagatagacatgggttcctttcagccctgagttcatgccagggttttctttccctctagctggactgaggtaggaggagaggttgaagtccac

SEQ ID NO: 286 - DNA, 120 bp, Homo sapiens
gctttcctcccttcctttccgagagtcctttagccaaatgccacacctcctcctgtttccctagtctccgtgcagagatggaagtgggagatagacatgggttcctttcagccctgagtt

SEQ ID NO: 287 - DNA, 120 bp, Homo sapiens
tccatcagccaaagcctcttgcctcccttcaacgtaactcttctctagcgtcctcttaataatcttctgaaaaggttttacagcctttctgggtactgggacccagagtcttaatccagg

SEQ ID NO: 288 - DNA, 120 bp, Homo sapiens
aatgagtttgggatgtgaatttcctaggcaacttgtcatctcttttttacttccttagcttcataaacttacccacaatgttccctgaggactaagagtaatggagggtgatgaggaaag

SEQ ID NO: 289 - DNA, 120 bp, Homo sapiens
aatcttctgaaaaggttttacagcctttctgggtactgggacccagagtcttaatccaggctcttaagtgccttatttaactgtaatatggaaaatcaaagtcacagctaattcaggaaa

SEQ ID NO: 290 - DNA, 120 bp, Homo sapiens
taccacaaactaaatcctatacttcagggataaaatcttctcctgttttttctaaaagcctgtgcatgtgtggtgtaaggggtgggttttcccttgtaccagcaacttagcaattgtagt

SEQ ID NO: 291 - DNA, 120 bp, Homo sapiens
tctagaagtaatttgtaaattcagaaagggcttatagatttaaagtgtagccgttttgattaccacaaactaaatcctatacttcagggataaaatcttctcctgttttttctaaaagcc

SEQ ID NO: 292 - DNA, 120 bp, Homo sapiens
aacggggctgagggcagtggcatgcttcttcattgagcaagtgtgaaaagagggttatgcattcaggggtcagcagatggcaggcagagtagcccctccaaatctccctcccataccaca

SEQ ID NO: 293 - DNA, 120 bp, Homo sapiens
tgtgcatgtgtggtgtaaggggtgggttttcccttgtaccagcaacttagcaattgtagtaacggggctgagggcagtggcatgcttcttcattgagcaagtgtgaaaagagggttatgc

SEQ ID NO: 294 - DNA, 120 bp, Homo sapiens
attcaggggtcagcagatggcaggcagagtagcccctccaaatctccctcccataccacaaagccctcttatttattcaaacttaacattagaagctcatttcaagtaggcacgtctgtg

SEQ ID NO: 295 - DNA, 120 bp, Homo sapiens
aagccctcttatttattcaaacttaacattagaagctcatttcaagtaggcacgtctgtgtctgggcgtctattttccttctttgtatatagcaggcatttgtcaacttggtgaaaagca

SEQ ID NO: 296 - DNA, 120 bp, Homo sapiens
tctgggcgtctattttccttctttgtatatagcaggcatttgtcaacttggtgaaaagcattactcttctttccatttctgaggactaattgtgcttcttcgctagacacgagttcaaaa

SEQ ID NO: 297 - DNA, 120 bp, Homo sapiens
cttatcttagtttgtatttctctgggtgtatatccctctcttgcagttctgggcctttgcagtttttggcttatgtttttgtatatatccactagaattggcttcttatcttttttgtgc

SEQ ID NO: 298 - DNA, 120 bp, Homo sapiens
ttgtactgcaaagtcttatttctgataagacatcacaaataagaattattgtgatgagacttatcacaaataagaattattgtgataattcttatttgtgataagaattactgggttaga

SEQ ID NO: 299 - DNA, 120 bp, Homo sapiens
cctgtgtgctttgctagtgggaacgtgtcttttcttttgtgctcgtatctctgtgtaatcgagtgtcttgctaagtcaatgtgcctctgtctctttttaccagttctgtctttgtgtctc

SEQ ID NO: 300 - DNA, 120 bp, Homo sapiens
aacttctgttcttttggtttctctttctttctttgattatattattttctggacttgttctgccaaagcaagaaggaaattccacatgtggctcactcatttattatacttgtttctttg

SEQ ID NO: 301 - DNA, 120 bp, Homo sapiens
tgtgccttcatgtattttttcccctgagtttgcacgtctctgtctatgtggatatctctcactccaggccactgtatcactgtgtctgtattacagctgtttatttctgtcggtgtgtgg

SEQ ID NO: 302 - DNA, 120 bp, Homo sapiens
gggggggggttgggggttgagcaattcattattattaaaatgcaaaaagcacttaattcgctatgataagattgcctttttcatgcatactggcctacctgcaagacccctagagacagt

SEQ ID NO: 303 - DNA, 120 bp, Homo sapiens
atttcagtttatgtcatagccattctttgtgtgactgcttctaggtatgtctttttctatgcccctattgtccccatctccatgtgtctctgtgtgtatatgttctaatgtatctgccta

SEQ ID NO: 304 - DNA, 120 bp, Homo sapiens
ctgcacatgtgttgtggtttgatgtttgtatgtgattgtgtaccagggtatgtgtgtctgttattgtgagttcatttctgagcagttgtgacacacagagatccagaaacagtgtcttac

SEQ ID NO: 305 - DNA, 120 bp, Homo sapiens
ggatggagtgcacaggaaatggagaggtgaaggtcatagagagaagtttagcaggaccagatctttccttgtcctgggctgctgtgaccatataaggaaggcagtaaggggaggggtagg

SEQ ID NO: 306 - DNA, 120 bp, Homo sapiens
agtgtgtctttattaacaggacacttgtgtgtagagaatccttgagaaatgagtggttagatgataaatcttttcatattaatttcatgatgtcagtgaagtaaatttgcaagatatggg

SEQ ID NO: 307 - DNA, 120 bp, Homo sapiens
gatttagttagaattattgaatgtaaaataatgaaaattaaaaaaaaaacaaggaggaggaatctatcctattttataattcagaccgttgaattgagtttttcttttgttgtattgatt

SEQ ID NO: 308 - DNA, 120 bp, Homo sapiens
tggtcattaataaaagactaggttcagtagaaacatgtaagttgtctaggtgttggaaattaatacagtactgtgctaagggaacatatatctagaagttaactgaattatgctcaataa

SEQ ID NO: 309 - DNA, 120 bp, Homo sapiens
gtatgttagtttgcttatctgcgccgtccctcctcttcccaacccactgtgtattgcagaatgttttatcagctctgatttgccaagttgctctcttctccagtaggtgctgcgagcaga

SEQ ID NO: 310 - DNA, 120 bp, Homo sapiens
atgtgtacgcatccatgtatctttctgcttctctgtgaccagatatttctgtgtagctgtctatgtatattggcttctgtctgtgtctgtgttgttggctctacgtctgtgcatatgcac

SEQ ID NO: 311 - DNA, 120 bp, Homo sapiens
atttctatgtctgttttcatcttaatttgtgtgtctaagcaagactgttttggggtgactatttcagtttatgtcatagccattctttgtgtgactgcttctaggtatgtctttttctat

SEQ ID NO: 312 - DNA, 120 bp, Homo sapiens
aatgggcgcaaccatgatttttctgatgtcagtctgtgatgtcagttgtccagtgtgtatgcaggctgcttaagagtacatacagttccttcacaattatggtagtccctgagaaggaag

SEQ ID NO: 313 - DNA, 120 bp, Homo sapiens
ctgcataagaactatgttctttttaaaactcagcatattgatggtggagaaagcatttatttgtactgcaaagtcttatttctgataagacatcacaaataagaattattgtgatgagac

SEQ ID NO: 314 - DNA, 120 bp, Homo sapiens
atgttttatcagctctgatttgccaagttgctctcttctccagtaggtgctgcgagcagagagggattcctcggaggtcatctgttccatcttcttgcctatgcaaatgcctgcctgaag

SEQ ID NO: 315 - DNA, 120 bp, Homo sapiens
cgtaggtgtgcataatttattcatgtaggatgtcaaaagagtcagttaaaaattatgcacagtgtgtctttattaacaggacacttgtgtgtagagaatccttgagaaatgagtggttag

SEQ ID NO: 316 - DNA, 120 bp, Homo sapiens
atatgggcagggggttctttatgccagttctgctctcttcccagtgtatctgtggtgcttaatgggcgcaaccatgatttttctgatgtcagtctgtgatgtcagttgtccagtgtgtat

SEQ ID NO: 317 - DNA, 120 bp, Homo sapiens
cacgatattaaagacagcttgttaagtgtcactgcaaacatcatacacactgatccactgatatgggcagggggttctttatgccagttctgctctcttcccagtgtatctgtggtgctt

SEQ ID NO: 318 - DNA, 120 bp, Homo sapiens
cttcaattgaaaacttagaactcagtttctagggtagtgagtgttgtaaggtttggactgtgacctaatattacgcagccatgacattatctattaggcatctagactagcttgcttgaa

SEQ ID NO: 319 - DNA, 120 bp, Homo sapiens
ctatgataagattgcctttttcatgcatactggcctacctgcaagacccctagagacagtaagcagcatacatggtgtcttccagttttcagcctttgtgcaaggaacaactgtgggttt

SEQ ID NO: 320 - DNA, 120 bp, Homo sapiens
tcagaggcctggcagtcagagggattctgatctctatatgcaatattttcacactactgtacttattgaaatcacatttgaatcttggcaattaacaaggcagtaattggcatcaggagg

SEQ ID NO: 321 - DNA, 120 bp, Homo sapiens
actccaggccactgtatcactgtgtctgtattacagctgtttatttctgtcggtgtgtggatttctatgtctgttttcatcttaatttgtgtgtctaagcaagactgttttggggtgact

SEQ ID NO: 322 - DNA, 120 bp, Homo sapiens
taaaattgacatgacaaaattaagtcattgtgtctgttttatgataaaacaggctcttttgatttagttagaattattgaatgtaaaataatgaaaattaaaaaaaaaacaaggaggagg

SEQ ID NO: 323 - DNA, 120 bp, Homo sapiens
gcaggctgcttaagagtacatacagttccttcacaattatggtagtccctgagaaggaagtggtcattaataaaagactaggttcagtagaaacatgtaagttgtctaggtgttggaaat

SEQ ID NO: 324 - DNA, 120 bp, Homo sapiens
aatctatcctattttataattcagaccgttgaattgagtttttcttttgttgtattgatttaaatgcagagaagtctatgatgctggattccagtcagaagataaacatttgtatgtggg

SEQ ID NO: 325 - DNA, 120 bp, Homo sapiens
aaagagtacaaatgtttcataaatattttgacctaatcctcctgtaagattaggagagggatatttccgatattcaaataatttttttaattggcaaacaccttagacatactatttaca

SEQ ID NO: 326 - DNA, 120 bp, Homo sapiens
tgtttgcagtcagttgtgttggctgtccctgtgtttgtccctgtgtgtgcatttcattgtatgtgtacgcatccatgtatctttctgcttctctgtgaccagatatttctgtgtagctgt

SEQ ID NO: 327 - DNA, 120 bp, Homo sapiens
gatgaggaagagaccagctctcctctttctttctgatggaaggttaccacctctatttaaaacttctgttcttttggtttctctttctttctttgattatattattttctggacttgttc

SEQ ID NO: 328 - DNA, 120 bp, Homo sapiens
gcccctattgtccccatctccatgtgtctctgtgtgtatatgttctaatgtatctgcctacttatcttagtttgtatttctctgggtgtatatccctctcttgcagttctgggcctttgc

SEQ ID NO: 329 - DNA, 120 bp, Homo sapiens
agtttttggcttatgtttttgtatatatccactagaattggcttcttatcttttttgtgcatgttttagtttgtatgagtgagcatatccaactctgtctttgagaagcagaactgtctg

SEQ ID NO: 330 - DNA, 120 bp, Homo sapiens
tgccaaagcaagaaggaaattccacatgtggctcactcatttattatacttgtttctttgcacgatattaaagacagcttgttaagtgtcactgcaaacatcatacacactgatccactg

SEQ ID NO: 331 - DNA, 120 bp, Homo sapiens
ctatgtatattggcttctgtctgtgtctgtgttgttggctctacgtctgtgcatatgcacccaccgggttcataaaaagctcacctgctctccaaggaatctaccagattattttgtgaa

SEQ ID NO: 332 - DNA, 120 bp, Homo sapiens
taatacagtactgtgctaagggaacatatatctagaagttaactgaattatgctcaataaaaagagtacaaatgtttcataaatattttgacctaatcctcctgtaagattaggagaggg

SEQ ID NO: 333 - DNA, 120 bp, Homo sapiens
atgttttagtttgtatgagtgagcatatccaactctgtctttgagaagcagaactgtctgtgtttgcagtcagttgtgttggctgtccctgtgtttgtccctgtgtgtgcatttcattgt

SEQ ID NO: 334 - DNA, 120 bp, Homo sapiens
tgctcctgacattgcctgtcactttttcccatgatactctggcttcacaggtgggaggttcttcaattgaaaacttagaactcagtttctagggtagtgagtgttgtaaggtttggactg

SEQ ID NO: 335 - DNA, 120 bp, Homo sapiens
tatcttagcatgttgactaatttggggcagaatatagtgtgggtgggggattttgtgtgtgggggggggttgggggttgagcaattcattattattaaaatgcaaaaagcacttaattcg

SEQ ID NO: 336 - DNA, 120 bp, Homo sapiens
ctgctggaggctggctttgtaccggactttgtacagggaaccagggaaacgaatgcagagtgctcctgacattgcctgtcactttttcccatgatactctggcttcacaggtgggaggtt

SEQ ID NO: 337 - DNA, 120 bp, Homo sapiens
tgacctaatattacgcagccatgacattatctattaggcatctagactagcttgcttgaatatcttagcatgttgactaatttggggcagaatatagtgtgggtgggggattttgtgtgt

SEQ ID NO: 338 - DNA, 120 bp, Homo sapiens
atctttccttgtcctgggctgctgtgaccatataaggaaggcagtaaggggaggggtagggatgaggaagagaccagctctcctctttctttctgatggaaggttaccacctctatttaa

SEQ ID NO: 339 - DNA, 120 bp, Homo sapiens
atgaaaagtaacagagagatgggcatattccttgtttgaatggagtcatccaggggctcaggatggagtgcacaggaaatggagaggtgaaggtcatagagagaagtttagcaggaccag

SEQ ID NO: 340 - DNA, 120 bp, Homo sapiens
acttattgaaatcacatttgaatcttggcaattaacaaggcagtaattggcatcaggagggtatgttagtttgcttatctgcgccgtccctcctcttcccaacccactgtgtattgcaga

SEQ ID NO: 341 - DNA, 120 bp, Homo sapiens
ccaccgggttcataaaaagctcacctgctctccaaggaatctaccagattattttgtgaaataactcacgtttcgtttttttacttgccagctgctatggtacttaaaagtgtgttggta

SEQ ID NO: 342 - DNA, 120 bp, Homo sapiens
aagcagcatacatggtgtcttccagttttcagcctttgtgcaaggaacaactgtgggtttctgcacatgtgttgtggtttgatgtttgtatgtgattgtgtaccagggtatgtgtgtctg

SEQ ID NO: 343 - DNA, 120 bp, Homo sapiens
atatttccgatattcaaataatttttttaattggcaaacaccttagacatactatttacataaaattgacatgacaaaattaagtcattgtgtctgttttatgataaaacaggctctttt

SEQ ID NO: 344 - DNA, 120 bp, Homo sapiens
tgttctttgatctcagtgattttgactctttctactgcactctggggacagtgggttctgcggtaccaactccaattaaagtgggaatatgtaccagcccctccccttggtttttatttt

SEQ ID NO: 345 - DNA, 120 bp, Homo sapiens
gagtgtcttgctaagtcaatgtgcctctgtctctttttaccagttctgtctttgtgtctctgtgccttcatgtattttttcccctgagtttgcacgtctctgtctatgtggatatctctc

SEQ ID NO: 346 - DNA, 120 bp, Homo sapiens
aggtgttacttttctggttttgtttgggtttttgttttgaagtgttactacagatggtgtcttagggacaaagagctctgaggttgacttagaacacatggagtacagataaaaaggaga

SEQ ID NO: 347 - DNA, 120 bp, Homo sapiens
ataactcacgtttcgtttttttacttgccagctgctatggtacttaaaagtgtgttggtacgtaggtgtgcataatttattcatgtaggatgtcaaaagagtcagttaaaaattatgcac

SEQ ID NO: 348 - DNA, 120 bp, Homo sapiens
atgataaatcttttcatattaatttcatgatgtcagtgaagtaaatttgcaagatatgggctgcataagaactatgttctttttaaaactcagcatattgatggtggagaaagcatttat

SEQ ID NO: 349 - DNA, 120 bp, Homo sapiens
cttagggacaaagagctctgaggttgacttagaacacatggagtacagataaaaaggagaatgaaaagtaacagagagatgggcatattccttgtttgaatggagtcatccaggggctca

SEQ ID NO: 350 - DNA, 120 bp, Homo sapiens
cggtaccaactccaattaaagtgggaatatgtaccagcccctccccttggtttttatttttcagaggcctggcagtcagagggattctgatctctatatgcaatattttcacactactgt

SEQ ID NO: 351 - DNA, 120 bp, Homo sapiens
ttattgtgagttcatttctgagcagttgtgacacacagagatccagaaacagtgtcttaccctgtgtgctttgctagtgggaacgtgtcttttcttttgtgctcgtatctctgtgtaatc

SEQ ID NO: 352 - DNA, 120 bp, Homo sapiens
gagggattcctcggaggtcatctgttccatcttcttgcctatgcaaatgcctgcctgaagctgctggaggctggctttgtaccggactttgtacagggaaccagggaaacgaatgcagag

SEQ ID NO: 353 - DNA, 120 bp, Homo sapiens
ttatcacaaataagaattattgtgataattcttatttgtgataagaattactgggttagaaggtgttacttttctggttttgtttgggtttttgttttgaagtgttactacagatggtgt

SEQ ID NO: 354 - DNA, 120 bp, Homo sapiens
atccgtggtaaaaattattaatgctttgcacatgcaacatagagtgttcaattttgttagtcaacaaatatttaagtggcagctgttatgacctcaggggtgtagtgacttccttattgt

SEQ ID NO: 355 - DNA, 120 bp, Homo sapiens
tgggtagtcaaaaatagttacatatacaagtcagcattttttaaattgttcagttgtgcttaagattggtcctttccaggaacaatccagctttatcaaaaaattattgcgtacatgtaa

SEQ ID NO: 356 - DNA, 120 bp, Homo sapiens
tgagattacttcttttcaggtaggaaactctactgtgtatttggctagttcaacctatcatgggtagtcaaaaatagttacatatacaagtcagcattttttaaattgttcagttgtgct

SEQ ID NO: 357 - DNA, 120 bp, Homo sapiens
cctttaattattaaaaaagaaatctatatcagaatatcaggtaaactcttattacatcaaatattataataaagatactttttatattctctaaacaaagtagagatctcagatgttggt

SEQ ID NO: 358 - DNA, 120 bp, Homo sapiens
tcatttatcaatataatattagatttgaaaattccagtatacaaaaggaaaaggacagcttcttaaagtttatagtgattttctatgaactatcaattccgtttttttctgttttactgg

SEQ ID NO: 359 - DNA, 120 bp, Homo sapiens
gcaaaccctggccattctgttttgtttaggaaagaattcatcagttctgattctgccttttctggggagggaggctgagtattggattgaagaggagtcactacttttctgagatgatat

SEQ ID NO: 360 - DNA, 120 bp, Homo sapiens
atattataataaagatactttttatattctctaaacaaagtagagatctcagatgttggttcatttatcaatataatattagatttgaaaattccagtatacaaaaggaaaaggacagct

SEQ ID NO: 361 - DNA, 120 bp, Homo sapiens
tctggggagggaggctgagtattggattgaagaggagtcactacttttctgagatgatatatccgtggtaaaaattattaatgctttgcacatgcaacatagagtgttcaattttgttag

SEQ ID NO: 362 - DNA, 120 bp, Homo sapiens
tcttaaagtttatagtgattttctatgaactatcaattccgtttttttctgttttactggtatgatggaaactaaatttcgagttgtaagtagtagataattagactgcagggtaagcct

SEQ ID NO: 363 - DNA, 120 bp, Homo sapiens
tatgatggaaactaaatttcgagttgtaagtagtagataattagactgcagggtaagccttgagattacttcttttcaggtaggaaactctactgtgtatttggctagttcaacctatca

SEQ ID NO: 364 - DNA, 120 bp, Homo sapiens
tcaacaaatatttaagtggcagctgttatgacctcaggggtgtagtgacttccttattgtcctttaattattaaaaaagaaatctatatcagaatatcaggtaaactcttattacatcaa

SEQ ID NO: 365 - DNA, 120 bp, Homo sapiens
acgtttatccatttagggacagcaggtttggcacaaatggattggttttctgaggtcttatgtagagggctgcactgactgacttctgaaagtcccccctaacccttcaaatctcagggt

SEQ ID NO: 366 - DNA, 120 bp, Homo sapiens
gctttgagttagaaagatagagtaagatggaggaaccaattcttccctgggttgatatttatttatcttgctcttttgaagtctaggccaatcatcctatttattctgaatggcccgtta

SEQ ID NO: 367 - DNA, 120 bp, Homo sapiens
aagctctacacttttagagggccattaacaatgctcaagttaaagaaaagcaatcaaagacaactaaaatactggtaccttcaaacagtacttatgaattatttaaccttagataatttg

SEQ ID NO: 368 - DNA, 120 bp, Homo sapiens
atttatcttgctcttttgaagtctaggccaatcatcctatttattctgaatggcccgttaacgtttatccatttagggacagcaggtttggcacaaatggattggttttctgaggtctta

SEQ ID NO: 369 - DNA, 120 bp, Homo sapiens
catctggtctcaagccttcaattatgaatacatttctattgcctttttgagtaacagcacaacactgcaagctgacccactgggtggatggaatggggctcttgccctaccaccctttgg

SEQ ID NO: 370 - DNA, 120 bp, Homo sapiens
catagttatttccctataatatttgttttatgattgtatagatgtctgctgaccaaccttaatctctgctccctaagattaaccattctacaaagcagaaactggaggtcattcaaatga

SEQ ID NO: 371 - DNA, 120 bp, Homo sapiens
caactaaaatactggtaccttcaaacagtacttatgaattatttaaccttagataatttggctttgagttagaaagatagagtaagatggaggaaccaattcttccctgggttgatattt

SEQ ID NO: 372 - DNA, 120 bp, Homo sapiens
tgtagagggctgcactgactgacttctgaaagtcccccctaacccttcaaatctcagggtcatctggtctcaagccttcaattatgaatacatttctattgcctttttgagtaacagcac

SEQ ID NO: 373 - DNA, 120 bp, Homo sapiens
aatctctgctccctaagattaaccattctacaaagcagaaactggaggtcattcaaatgaaagctctacacttttagagggccattaacaatgctcaagttaaagaaaagcaatcaaaga

SEQ ID NO: 374 - DNA, 120 bp, Homo sapiens
cttcgaatttttgtcaaaaagtattctttcattagaaagatacatgggtgtgcttccatgtcagcaacatgactgcagaccaggaagtcctcacggagagctggaatatgggtattttgg

SEQ ID NO: 375 - DNA, 120 bp, Homo sapiens
ctcccaccctctaaaacaacagaggcagcaaccatttacacactttccagaagtaagtaagtaagactgtattccagaaacaccctatatcaaaatggaaatatactcaagtgccccaat

SEQ ID NO: 376 - DNA, 120 bp, Homo sapiens
gtaagactgtattccagaaacaccctatatcaaaatggaaatatactcaagtgccccaatgacccattgggctagtttgaacgtgtgcagtctctgtgctccccgttttagcttaagcct

SEQ ID NO: 377 - DNA, 120 bp, Homo sapiens
actgagcagtgggagtggagcaggttggggatagtgaagtatttgtaattcatttttaaaaaggagagggagagagaaaaggaaaaactgggccacccatcctttgaaaagaaaccttga

SEQ ID NO: 378 - DNA, 120 bp, Homo sapiens
tagcttgctagtaacttcacatgctatttcctttacctcttatatttgaggtgtctatttggagtgggctgtgtttctagctattctgtttatctggtttgtttttgttggtgtaggaaa

SEQ ID NO: 379 - DNA, 120 bp, Homo sapiens
ttatgagaaaaaaagtcaaacaaacatatttgaaatgtccagaaaacctgtgagtttttatgtatactatacaggaaagatattctgtcatctggttgccaaactatggagggtgggaga

SEQ ID NO: 380 - DNA, 120 bp, Homo sapiens
ttgctggtggatatggtggaataccttttatgtggttatctcctccttgtaactcttggctgcataacccttattttcttttctatttttattctctctcttggaaaaaaaattggtggt

SEQ ID NO: 381 - DNA, 120 bp, Homo sapiens
agagggtagtttgaaagaaaatgctagtgactacgtgtgtttccttcctgacatattttatagaaggtgatgagttccagcattttttcagacttggatctggctttcattccccttctc

SEQ ID NO: 382 - DNA, 120 bp, Homo sapiens
ctttgtaggacatatgatctttgctaagtgcactgaatgtatgtagaggagacaagtctgctgagggtatgagaattgggccaagatttaacacattttcaaagctccatgaagaagcct

SEQ ID NO: 383 - DNA, 120 bp, Homo sapiens
gggatagcattaaaagaaatagtgcttttgtttagaagaagaaatgaaatgcttgtgtccagatgcttaaaggaaggcagtgcagactttcagaaactagactttaagagctgtactcag

SEQ ID NO: 384 - DNA, 120 bp, Homo sapiens
actctctggttagatgcagcttttacttcacatcctcagtggtactactgtaaattttcattttcctgtggaataccctatttggttccattgtatatagttgacaactagaattcgttc

SEQ ID NO: 385 - DNA, 120 bp, Homo sapiens
ctgagggtatgagaattgggccaagatttaacacattttcaaagctccatgaagaagcctactgagcagtgggagtggagcaggttggggatagtgaagtatttgtaattcatttttaaa

SEQ ID NO: 386 - DNA, 120 bp, Homo sapiens
agcggcatttttttacttctcaatatgaggttgaaactataagcttaaattgctgactttctggcagcaccaaacagtaaggaaaccacaaagataaacccaaataatagagccaatttt

SEQ ID NO: 387 - DNA, 120 bp, Homo sapiens
tagaaggtgatgagttccagcattttttcagacttggatctggctttcattccccttctcctcccaccctctaaaacaacagaggcagcaaccatttacacactttccagaagtaagtaa

SEQ ID NO: 388 - DNA, 120 bp, Homo sapiens
gctacaattaatccaagcaaagggaaagatgtcagtaaaactgccccttttcatagaggtgtggcaactgctgggaaggaagaaattagcctgaggccatgtgattactaataaactcaa

SEQ ID NO: 389 - DNA, 120 bp, Homo sapiens
ggaagagctaccataatgaatgtgtacatggacaaaaaaaaagagagagagagagagaattaaatcatgagtttgtgccttgggagctacagtttaaacatttgctgtttttctcactta

SEQ ID NO: 390 - DNA, 120 bp, Homo sapiens
tttagtaaaatggccaccagaaataaaggattttattttccagactttggtgtttggagctggtgtgctgagagctagcagagaaagccctactcaggtagatgtaccagagcaggatgg

SEQ ID NO: 391 - DNA, 120 bp, Homo sapiens
tggtgtgctgagagctagcagagaaagccctactcaggtagatgtaccagagcaggatggttgctggtggatatggtggaataccttttatgtggttatctcctccttgtaactcttggc

SEQ ID NO: 392 - DNA, 120 bp, Homo sapiens
ctttttttccgggggggatgacttctaactagtgatatgaggaaggataagaaaatgtttctttgtaggacatatgatctttgctaagtgcactgaatgtatgtagaggagacaagtctg

SEQ ID NO: 393 - DNA, 120 bp, Homo sapiens
gctgttgcttgagcccaactataacttcttggcactatacctatcttctgatgtgcctgtggaagagctaccataatgaatgtgtacatggacaaaaaaaaagagagagagagagagaat

SEQ ID NO: 394 - DNA, 120 bp, Homo sapiens
gtggcaactgctgggaaggaagaaattagcctgaggccatgtgattactaataaactcaaagcggcatttttttacttctcaatatgaggttgaaactataagcttaaattgctgacttt

SEQ ID NO: 395 - DNA, 120 bp, Homo sapiens
agaaataattgtaggtagcttagccttggctgtagtcagaacttttgtactgtgactttaggatctgtatggaatcgtatgatatgcggatacaccaaaaactctatgggttatcaaaat

SEQ ID NO: 396 - DNA, 120 bp, Homo sapiens
ggagtgggctgtgtttctagctattctgtttatctggtttgtttttgttggtgtaggaaactggtataaattttatttgggtaaatatcacctcaattttcaactaaagctttatttaag

SEQ ID NO: 397 - DNA, 120 bp, Homo sapiens
tttcacatgaaaaagacaaatgaggcaaaggaagagaaaaatgcattgtcagaatcagaattatgagaaaaaaagtcaaacaaacatatttgaaatgtccagaaaacctgtgagttttta

SEQ ID NO: 398 - DNA, 120 bp, Homo sapiens
atactgagaagggctgatggctgaaggaggaacaatttaaaagaataaccgtctctcctctccctgtatattggacataaaagaatatcccattcttttcagaaatgtaatacaacagtt

SEQ ID NO: 399 - DNA, 120 bp, Homo sapiens
tccctgtatattggacataaaagaatatcccattcttttcagaaatgtaatacaacagtttagcttgctagtaacttcacatgctatttcctttacctcttatatttgaggtgtctattt

SEQ ID NO: 400 - DNA, 120 bp, Homo sapiens
aaggagagggagagagaaaaggaaaaactgggccacccatcctttgaaaagaaaccttgaaagaggtccaaatatccttagaaatccttgacttcttaaaagtgatgtttgttttttccc

SEQ ID NO: 401 - DNA, 120 bp, Homo sapiens
tctgactttatggcctctcagctttcaatgactagctttgtagcagaagtttagcctctcatccccataactttggaagtagtgttgagataaagaaacgttgaattgaaggttgtgttt

SEQ ID NO: 402 - DNA, 120 bp, Homo sapiens
tgtatactatacaggaaagatattctgtcatctggttgccaaactatggagggtgggagacttcgaatttttgtcaaaaagtattctttcattagaaagatacatgggtgtgcttccatg

SEQ ID NO: 403 - DNA, 120 bp, Homo sapiens
ctcctcctaatacacttatccacgtttggataccttggtctcagcctcagaggtcatatttttagtaaaatggccaccagaaataaaggattttattttccagactttggtgtttggagc

SEQ ID NO: 404 - DNA, 120 bp, Homo sapiens
ctggtataaattttatttgggtaaatatcacctcaattttcaactaaagctttatttaagtttcacatgaaaaagacaaatgaggcaaaggaagagaaaaatgcattgtcagaatcagaa

SEQ ID NO: 405 - DNA, 120 bp, Homo sapiens
ttcttgcaagtggtggtttacttcacttcaactatagaaggcctatgcgacaccacccatagagggtagtttgaaagaaaatgctagtgactacgtgtgtttccttcctgacatatttta

SEQ ID NO: 406 - DNA, 120 bp, Homo sapiens
tcagcaacatgactgcagaccaggaagtcctcacggagagctggaatatgggtattttggactctctggttagatgcagcttttacttcacatcctcagtggtactactgtaaattttca

SEQ ID NO: 407 - DNA, 120 bp, Homo sapiens
tgtacggaaaataggagttgataatttttaaggcccttgccagcacattagtacataggattcttgcaagtggtggtttacttcacttcaactatagaaggcctatgcgacaccacccat

SEQ ID NO: 408 - DNA, 120 bp, Homo sapiens
aagaggtccaaatatccttagaaatccttgacttcttaaaagtgatgtttgttttttccccctgacaattatagaggtcagagagtttttcttttctattacaaaacattgagagtgtgt

SEQ ID NO: 409 - DNA, 120 bp, Homo sapiens
atgaaaaatttatttgaaaataacagcacagaaaggaagaaagacaggctggcaagcatcctcctcctaatacacttatccacgtttggataccttggtctcagcctcagaggtcatatt

SEQ ID NO: 410 - DNA, 120 bp, Homo sapiens
cctgacaattatagaggtcagagagtttttcttttctattacaaaacattgagagtgtgtagaaataattgtaggtagcttagccttggctgtagtcagaacttttgtactgtgacttta

SEQ ID NO: 411 - DNA, 120 bp, Homo sapiens
agacaaaattctttccattctccagcttatattttccccatttgtaaaacataatggaagtgtacggaaaataggagttgataatttttaaggcccttgccagcacattagtacatagga

SEQ ID NO: 412 - DNA, 120 bp, Homo sapiens
tctagatttctttcaattgctccttaggctttagaagataaattctcctaaaagagaggtgctacaattaatccaagcaaagggaaagatgtcagtaaaactgccccttttcatagaggt

SEQ ID NO: 413 - DNA, 120 bp, Homo sapiens
ttttcctgtggaataccctatttggttccattgtatatagttgacaactagaattcgttcgctgttgcttgagcccaactataacttcttggcactatacctatcttctgatgtgcctgt

SEQ ID NO: 414 - DNA, 120 bp, Homo sapiens
gacccattgggctagtttgaacgtgtgcagtctctgtgctccccgttttagcttaagcctactccctaacctgtcatatgtcacccagccatggagcctagggcaatgactgccatcata

SEQ ID NO: 415 - DNA, 120 bp, Homo sapiens
agatgcttaaaggaaggcagtgcagactttcagaaactagactttaagagctgtactcagatactgagaagggctgatggctgaaggaggaacaatttaaaagaataaccgtctctcctc

SEQ ID NO: 416 - DNA, 120 bp, Homo sapiens
actccctaacctgtcatatgtcacccagccatggagcctagggcaatgactgccatcatatctgactttatggcctctcagctttcaatgactagctttgtagcagaagtttagcctctc

SEQ ID NO: 417 - DNA, 120 bp, Homo sapiens
taaatcatgagtttgtgccttgggagctacagtttaaacatttgctgtttttctcacttaatgaaaaatttatttgaaaataacagcacagaaaggaagaaagacaggctggcaagcatc

SEQ ID NO: 418 - DNA, 120 bp, Homo sapiens
atccccataactttggaagtagtgttgagataaagaaacgttgaattgaaggttgtgttttctagatttctttcaattgctccttaggctttagaagataaattctcctaaaagagaggt

SEQ ID NO: 419 - DNA, 120 bp, Homo sapiens
ctggcagcaccaaacagtaaggaaaccacaaagataaacccaaataatagagccaattttctttttttccgggggggatgacttctaactagtgatatgaggaaggataagaaaatgttt

SEQ ID NO: 420 - DNA, 120 bp, Homo sapiens
ggatctgtatggaatcgtatgatatgcggatacaccaaaaactctatgggttatcaaaatgggatagcattaaaagaaatagtgcttttgtttagaagaagaaatgaaatgcttgtgtcc

SEQ ID NO: 421 - DNA, 120 bp, Homo sapiens
gtctctatgtacttgtgtgtgaaacaatgagcacaaataataccctccttgtttttaagcaatttatattggtgatttaaaaataaaataaactcaagtgggaaatcatgaaaccccatg

SEQ ID NO: 422 - DNA, 120 bp, Homo sapiens
actgagaatgcaacccaagaacagaaatttgtcagaaatttagcactgaagccccccacttcccaaacttatctgggacaaggagaatctacatttaaagctctatactttgtgttgtgt

SEQ ID NO: 423 - DNA, 120 bp, Homo sapiens
tgcattagccactttgctgggtatgaatgccagcagaatctaagtgaccttggcttcactactgagaatgcaacccaagaacagaaatttgtcagaaatttagcactgaagccccccact

SEQ ID NO: 424 - DNA, 120 bp, Homo sapiens
cctaagcagaggcaagggaggaaagggatatgaacctggtagaaaagtaagtaagctttattcagattggcatatccatcttaatatggttcaattggctgaagaagtatctcaactaaa

SEQ ID NO: 425 - DNA, 120 bp, Homo sapiens
tcccaaacttatctgggacaaggagaatctacatttaaagctctatactttgtgttgtgttttttttactttagcttggttggatttaggatcttttctttttgttttgccttatgcata

SEQ ID NO: 426 - DNA, 120 bp, Homo sapiens
tttttttactttagcttggttggatttaggatcttttctttttgttttgccttatgcatacctaagcagaggcaagggaggaaagggatatgaacctggtagaaaagtaagtaagcttta

SEQ ID NO: 427 - DNA, 120 bp, Homo sapiens
agttatatgatgttttctagcaagcatttgcgttgttctactggtgttacatatcttagctgcattagccactttgctgggtatgaatgccagcagaatctaagtgaccttggcttcact

SEQ ID NO: 428 - DNA, 120 bp, Homo sapiens
ttcagattggcatatccatcttaatatggttcaattggctgaagaagtatctcaactaaaactctggaatactttgaagtaccagcaatatgtaccaaatgtactttttatttatgtttg

SEQ ID NO: 429 - DNA, 120 bp, Homo sapiens
actctggaatactttgaagtaccagcaatatgtaccaaatgtactttttatttatgtttggtctctatgtacttgtgtgtgaaacaatgagcacaaataataccctccttgtttttaagc

SEQ ID NO: 430 - DNA, 120 bp, Homo sapiens
actttgaacatttatattctgctttatttaggcataagtgcttaataattattgatagtttcttctggttatctgacattttgaagatactattacctagcagaaatttcttgtaataat

SEQ ID NO: 431 - DNA, 120 bp, Homo sapiens
ttgcagtcaaatctggcatgaaattagtgcatagacagaatgggctgggaaaatgaaaggactttgaacatttatattctgctttatttaggcataagtgcttaataattattgatagtt

SEQ ID NO: 432 - DNA, 120 bp, Homo sapiens
agtgttcatattttgtatttgagcacagggcaactgggtttttgaaactgcacattactgttgcagtcaaatctggcatgaaattagtgcatagacagaatgggctgggaaaatgaaagg

SEQ ID NO: 433 - DNA, 120 bp, Homo sapiens
agttcatcttaagcatatggctgtctgtcttttctctaaagatcctcaagggaaaaaaaaaaaagcatctccagggggaatttactgcctcatagccctgacagagatttctgaccaaac

SEQ ID NO: 434 - DNA, 120 bp, Homo sapiens
cttgaatatccttttagcgacttgcctttggtgttagtgtgcctataacattgtcgttgaatatcttaatacatttagtggtcttggcaagcagttttgtcttcagaaggacactgaaat

SEQ ID NO: 435 - DNA, 120 bp, Homo sapiens
atatcttaatacatttagtggtcttggcaagcagttttgtcttcagaaggacactgaaatctgtggaaaggactgcagaagattgggtgggcagacacctatcactttcggggctggtag

SEQ ID NO: 436 - DNA, 120 bp, Homo sapiens
actttctattgaagcaatttgcaaggctactttgtattgtctaaaagcactacttcagaaaagggttgtgatgtcaaaataggcactttgagtgaagaaagggctgtaagcatgggtgga

SEQ ID NO: 437 - DNA, 120 bp, Homo sapiens
aaaatatacacagctttggatttccttattatggcccttcattaagttgtggtttaagaatagctatgattattacttttgtgataattataatccataatatggaaacttataaaatta

SEQ ID NO: 438 - DNA, 120 bp, Homo sapiens
aaatgtggtagatgattgtcttgagttattttctttaatgtcaaacaggcagtccttggaatgctacttcaaaaagtgttgtataatgttgaagatacagttacagatttccaacacgaa

SEQ ID NO: 439 - DNA, 120 bp, Homo sapiens
cctaacgaaaaaatttcttccctccatttgtcttttattgtttttacaggggagatatgtaacataataacaattatattgcacataataattacttctacaaataataatctgttgtca

SEQ ID NO: 440 - DNA, 120 bp, Homo sapiens
aacataataacaattatattgcacataataattacttctacaaataataatctgttgtcaaaaatatacacagctttggatttccttattatggcccttcattaagttgtggtttaagaa

SEQ ID NO: 441 - DNA, 120 bp, Homo sapiens
tctctgagtagtaccataagttgttagtctgctactctttctcccagttggcacatgaccctaacatccaatcgctagtggtgtggccattttttggtcttattttggcctttcctcagc

SEQ ID NO: 442 - DNA, 120 bp, Homo sapiens
cagcctttttgcacaacaaaatggcagcacccaggaggttgaaagggttaaattgttccttctctgagtagtaccataagttgttagtctgctactctttctcccagttggcacatgacc

SEQ ID NO: 443 - DNA, 120 bp, Homo sapiens
tagctatgattattacttttgtgataattataatccataatatggaaacttataaaattacctttaaagtgttactattattctggccacaggatggaaagttgttcgctagttactcat

SEQ ID NO: 444 - DNA, 120 bp, Homo sapiens
atgctacttcaaaaagtgttgtataatgttgaagatacagttacagatttccaacacgaaactcataaatatgcaattccctgtcctcctaggcacatgaaggaaaatttatgagcttca

SEQ ID NO: 445 - DNA, 120 bp, Homo sapiens
ggtttctatgcagctattaaagcatatttaatctgctttgagctcaagctcactctcgttggctctcttcgtttcttcctcttacatgagcaaactgcctttctttttgtttaaaaatag

SEQ ID NO: 446 - DNA, 120 bp, Homo sapiens
aagggttgtgatgtcaaaataggcactttgagtgaagaaagggctgtaagcatgggtggaaaatgtggtagatgattgtcttgagttattttctttaatgtcaaacaggcagtccttgga

SEQ ID NO: 447 - DNA, 120 bp, Homo sapiens
actcataaatatgcaattccctgtcctcctaggcacatgaaggaaaatttatgagcttcaggtttctatgcagctattaaagcatatttaatctgctttgagctcaagctcactctcgtt

SEQ ID NO: 448 - DNA, 120 bp, Homo sapiens
cctttaaagtgttactattattctggccacaggatggaaagttgttcgctagttactcatttataacctgaatgtactttttactgaatctaaaggtatcatctttgcttggcaattccc

SEQ ID NO: 449 - DNA, 120 bp, Homo sapiens
ctaacatccaatcgctagtggtgtggccattttttggtcttattttggcctttcctcagccaccactcatcagttctcatgcgtatttgtcagatcctgctccccaactccacagttctt

SEQ ID NO: 450 - DNA, 120 bp, Homo sapiens
taagtaggtttgttttcctccaggtgtcatgaatgcaaacattgtaatttctcatctgttcagcctttttgcacaacaaaatggcagcacccaggaggttgaaagggttaaattgttcct

SEQ ID NO: 451 - DNA, 120 bp, Homo sapiens
caccactcatcagttctcatgcgtatttgtcagatcctgctccccaactccacagttcttagttcatcttaagcatatggctgtctgtcttttctctaaagatcctcaagggaaaaaaaa

SEQ ID NO: 452 - DNA, 120 bp, Homo sapiens
aaaagcatctccagggggaatttactgcctcatagccctgacagagatttctgaccaaaccctaacgaaaaaatttcttccctccatttgtcttttattgtttttacaggggagatatgt

SEQ ID NO: 453 - DNA, 120 bp, Homo sapiens
ctgtggaaaggactgcagaagattgggtgggcagacacctatcactttcggggctggtagactttctattgaagcaatttgcaaggctactttgtattgtctaaaagcactacttcagaa

SEQ ID NO: 454 - DNA, 120 bp, Homo sapiens
ggctctcttcgtttcttcctcttacatgagcaaactgcctttctttttgtttaaaaatagtaagtaggtttgttttcctccaggtgtcatgaatgcaaacattgtaatttctcatctgtt

SEQ ID NO: 455 - DNA, 120 bp, Homo sapiens
tatcatgttcaggccttctttccagcatgtggctctcagccctggtactgtccttaaccataaacctcatctttgccctctatagggagaggtttatggttataattactcattttaaat

SEQ ID NO: 456 - DNA, 120 bp, Homo sapiens
cttcaagaagtttttacgtacctcttatatagaatgtgatgttttatatgtacctcttatagaatgtgagcttttaagaggcatatcttattgcaagaaatttcaatgttgaaaaaaata

SEQ ID NO: 457 - DNA, 120 bp, Homo sapiens
acaaatctttaattcacatggcaatttctaggatttatcatggaaaatgagccaaattgccttcaagaagtttttacgtacctcttatatagaatgtgatgttttatatgtacctcttat

SEQ ID NO: 458 - DNA, 120 bp, Homo sapiens
ttgaatatttataaagtcaaaaatgcaaacttttatatgattttcaaacctatgaagttatatcatgttcaggccttctttccagcatgtggctctcagccctggtactgtccttaacca

SEQ ID NO: 459 - DNA, 120 bp, Homo sapiens
agaatgtgagcttttaagaggcatatcttattgcaagaaatttcaatgttgaaaaaaatattgaatatttataaagtcaaaaatgcaaacttttatatgattttcaaacctatgaagtta

SEQ ID NO: 460 - DNA, 120 bp, Homo sapiens
gagtcaactgtaagtagacagaattgcctttgacttaatctgtttcagtcgttgttcatactcaggtcctccagaggacctttaagcatttttattgactttgtggtctattacacgaaa

SEQ ID NO: 461 - DNA, 120 bp, Homo sapiens
gagcacataacattcttttgtgctaacagtatctctgcatcacattgatcaggagaattggcatctccagagccctgggatggtaacttctctgttgattttcaggaaagattaggtgat

SEQ ID NO: 462 - DNA, 120 bp, Homo sapiens
agaaattattagttaaagaaataatagaattttacaagactctaggaagggagaatgtgaaggatacagttctcagttactggaatgagtgccagagtaccagtacatggcttgccttgg

SEQ ID NO: 463 - DNA, 120 bp, Homo sapiens
tcagattcagttaaagtttactttcctgacagctttttagtatcatatctattttgcaaaactctagtgataaatgtatgcacatttacacatacagcatctcttctgattctgactaag

SEQ ID NO: 464 - DNA, 120 bp, Homo sapiens
gcatctccagagccctgggatggtaacttctctgttgattttcaggaaagattaggtgatattttctccatgggaagaggatgtttgatgtgtgttggctttagcaaaaggaagcttgtg

SEQ ID NO: 465 - DNA, 120 bp, Homo sapiens
ggtttggactacctatcttaactcctttgctcctcccaatcttgatctcatttgtttgaaagatcatctgcccaacataaaaatgcatttctaattctgtaatttaagtcagtggcaaga

SEQ ID NO: 466 - DNA, 120 bp, Homo sapiens
atctaaggactgaattttctcatctttgtctttgccccttttgactgatgaccagagcaggagcacataacattcttttgtgctaacagtatctctgcatcacattgatcaggagaattg

SEQ ID NO: 467 - DNA, 120 bp, Homo sapiens
attttctccatgggaagaggatgtttgatgtgtgttggctttagcaaaaggaagcttgtggagtcaactgtaagtagacagaattgcctttgacttaatctgtttcagtcgttgttcata

SEQ ID NO: 468 - DNA, 120 bp, Homo sapiens
aggatacagttctcagttactggaatgagtgccagagtaccagtacatggcttgccttggggtttggactacctatcttaactcctttgctcctcccaatcttgatctcatttgtttgaa

SEQ ID NO: 469 - DNA, 120 bp, Homo sapiens
atattactgggttgtgtagaagtgatgggctctttagaagaaaggtttgatatactactaatctaaggactgaattttctcatctttgtctttgccccttttgactgatgaccagagcag

SEQ ID NO: 470 - DNA, 120 bp, Homo sapiens
gtgagaaaaaagcagcatccataaggttttcattctcctaccctgtacgacagaggtaatagaaattattagttaaagaaataatagaattttacaagactctaggaagggagaatgtga

SEQ ID NO: 471 - DNA, 120 bp, Homo sapiens
agatcatctgcccaacataaaaatgcatttctaattctgtaatttaagtcagtggcaagatcagattcagttaaagtttactttcctgacagctttttagtatcatatctattttgcaaa

SEQ ID NO: 472 - DNA, 120 bp, Homo sapiens
actctagtgataaatgtatgcacatttacacatacagcatctcttctgattctgactaagatattactgggttgtgtagaagtgatgggctctttagaagaaaggtttgatatactacta

SEQ ID NO: 473 - DNA, 120 bp, Homo sapiens
aaaagaaccacattctattcacatgcttcattttattctgatttatgtaaaaattcccaaactcctcaagcagtgtttctttgtaaggcaataatcttcagttctgttgcaaaggtcagg

SEQ ID NO: 474 - DNA, 120 bp, Homo sapiens
tgggcctagagggttatataagatttcaatattaaaacatggattaaaagtgaagacttttcacatggagataatttggaagaaaaacttgcaaaaatgtgagagcattgagaacttttc

SEQ ID NO: 475 - DNA, 120 bp, Homo sapiens
ctactatgaaatctctgttcccagctagaggcctgggagagtaagataactacttgtttattccacggagccacttattagctttttctatagcacatacctcaaatgaagcatttcaat

SEQ ID NO: 476 - DNA, 120 bp, Homo sapiens
actcctcaagcagtgtttctttgtaaggcaataatcttcagttctgttgcaaaggtcaggagtgatagaatgaaaatggtactagatacaacagctctttggtatttgcatggccattac

SEQ ID NO: 477 - DNA, 120 bp, Homo sapiens
tttcccaaggaaagaagtggcagcttcatttttggtcattgcaaacagcagtgccatacatgaaaggaaagtggtggtgctcatcaactttgaataactttgtacagaacccttgagact

SEQ ID NO: 478 - DNA, 120 bp, Homo sapiens
tttgttactacttattggggatcctggaaagaaaatatattgtctatatccactgttcactgaggccctctccctacccagaaactccctgtctccatcactcactctccacattcattg

SEQ ID NO: 479 - DNA, 120 bp, Homo sapiens
tcacatggagataatttggaagaaaaacttgcaaaaatgtgagagcattgagaacttttctttcccaaggaaagaagtggcagcttcatttttggtcattgcaaacagcagtgccataca

SEQ ID NO: 480 - DNA, 120 bp, Homo sapiens
aggtagttctcaaatatttactccatgaatagttgctggatgttcattaactctatagcatttgttactacttattggggatcctggaaagaaaatatattgtctatatccactgttcac

SEQ ID NO: 481 - DNA, 120 bp, Homo sapiens
atttcaaggcaagaggtataagagaaagttcagagacaacactggctatggtctttgtgaagaaaagtgaattgaataggctcctgtggagatcttaagtaagtacttctggagataagg

SEQ ID NO: 482 - DNA, 120 bp, Homo sapiens
cctctctgcttataaagaaaaagtgtcaactgtaaagttgatttatttatgaaccataggctactatgaaatctctgttcccagctagaggcctgggagagtaagataactacttgttta

SEQ ID NO: 483 - DNA, 120 bp, Homo sapiens
ctgacccctagaaaatcctggtgaagtttttctggtgtcagtttggtcttaatgtttaggaaatgcccacagactactcctgctttctgcttattcacatagtaaacgcaaagcacagga

SEQ ID NO: 484 - DNA, 120 bp, Homo sapiens
agaaaagtgaattgaataggctcctgtggagatcttaagtaagtacttctggagataaggttgaggaaaagtaggtttgaatcttcatccagaggtagcccctaaatgtgttgagtttat

SEQ ID NO: 485 - DNA, 120 bp, Homo sapiens
tgaaaggaaagtggtggtgctcatcaactttgaataactttgtacagaacccttgagactcctctctgcttataaagaaaaagtgtcaactgtaaagttgatttatttatgaaccatagg

SEQ ID NO: 486 - DNA, 120 bp, Homo sapiens
acccaggggaacagttcatggatgagtgaacttgagctctatcttaaaggatggagttcgatttcaaggcaagaggtataagagaaagttcagagacaacactggctatggtctttgtga

SEQ ID NO: 487 - DNA, 120 bp, Homo sapiens
tattcaacaaccataaaaaaaaacaattaggactcaagtagtatgtcagagtgtagtcactgatgatatataattctccactaccaagaagatggaagcacactgttgagtagctacatc

SEQ ID NO: 488 - DNA, 120 bp, Homo sapiens
ctacatatgttggccagaatttaggaatacacatgtgatctatacattttgaggtattgtctgacccctagaaaatcctggtgaagtttttctggtgtcagtttggtcttaatgtttagg

SEQ ID NO: 489 - DNA, 120 bp, Homo sapiens
ttccacggagccacttattagctttttctatagcacatacctcaaatgaagcatttcaataaaagaaccacattctattcacatgcttcattttattctgatttatgtaaaaattcccaa

SEQ ID NO: 490 - DNA, 120 bp, Homo sapiens
tgtgtttgtgctaatggagtgatttgagaggtagttctccactgtcagtcaagaggttggttttgaaagctgattgccaatggtcattctgctaaccactctggttctcctttagataga

SEQ ID NO: 491 - DNA, 120 bp, Homo sapiens
tgatgatatataattctccactaccaagaagatggaagcacactgttgagtagctacatcctacatatgttggccagaatttaggaatacacatgtgatctatacattttgaggtattgt

SEQ ID NO: 492 - DNA, 120 bp, Homo sapiens
gacttattcagattcaagtcttcatgtactttgtggcataaacattgtacacaccagatgtattcaacaaccataaaaaaaaacaattaggactcaagtagtatgtcagagtgtagtcac

SEQ ID NO: 493 - DNA, 120 bp, Homo sapiens
aaatgcccacagactactcctgctttctgcttattcacatagtaaacgcaaagcacaggactagtttgtcatctggatcaaggagaaatgagttagcagatataaaataaatcagaaagg

SEQ ID NO: 494 - DNA, 120 bp, Homo sapiens
gtggctgaacttctccactcccgtatccatcgcaatacttccccaaggtggcatttaagatgggcctagagggttatataagatttcaatattaaaacatggattaaaagtgaagacttt

SEQ ID NO: 495 - DNA, 120 bp, Homo sapiens
attgccatggggctgcaagacttgtgagtgcttgatattttgcttgttgatgaatgagtctgtgtttgtgctaatggagtgatttgagaggtagttctccactgtcagtcaagaggttgg

SEQ ID NO: 496 - DNA, 120 bp, Homo sapiens
ttttgaaagctgattgccaatggtcattctgctaaccactctggttctcctttagatagagacttattcagattcaagtcttcatgtactttgtggcataaacattgtacacaccagatg

SEQ ID NO: 497 - DNA, 120 bp, Homo sapiens
agtgatagaatgaaaatggtactagatacaacagctctttggtatttgcatggccattacattgccatggggctgcaagacttgtgagtgcttgatattttgcttgttgatgaatgagtc

SEQ ID NO: 498 - DNA, 120 bp, Homo sapiens
tgaggccctctccctacccagaaactccctgtctccatcactcactctccacattcattgacccaggggaacagttcatggatgagtgaacttgagctctatcttaaaggatggagttcg

SEQ ID NO: 499 - DNA, 120 bp, Homo sapiens
ctagtttgtcatctggatcaaggagaaatgagttagcagatataaaataaatcagaaaggaggtagttctcaaatatttactccatgaatagttgctggatgttcattaactctatagca

SEQ ID NO: 500 - DNA, 120 bp, Homo sapiens
tgtcttccaactctcccaggcttctttgtttgcaaggctgacttttataatactttttgggtagagcaggtccttctttggtttggggttaaaccgtgagtaaccttattttctaggtct

SEQ ID NO: 501 - DNA, 120 bp, Homo sapiens
gccaagcactcagtaaatgtttgaatgggaaaattaactgccctgtttttctattgtcagatggtcctcttcgttggataacttggtaactgttgataaccttttctcaggaatcagaag

SEQ ID NO: 502 - DNA, 120 bp, Homo sapiens
gtagaaaggttgggaaaatataagaaacaaaaaggcatattcctatttttattttcatattgtcttccaactctcccaggcttctttgtttgcaaggctgacttttataatactttttgg

SEQ ID NO: 503 - DNA, 120 bp, Homo sapiens
gtagagcaggtccttctttggtttggggttaaaccgtgagtaaccttattttctaggtctcagccaactttgaagggcatgaactcacagtagcctcactaggatcacttcagcagtgag

SEQ ID NO: 504 - DNA, 120 bp, Homo sapiens
atggtcctcttcgttggataacttggtaactgttgataaccttttctcaggaatcagaaggtagaaaggttgggaaaatataagaaacaaaaaggcatattcctatttttattttcatat

SEQ ID NO: 505 - DNA, 120 bp, Homo sapiens
aatatggactgagtttctgtggggtggaaatgtgaagtggatcatagcatgatataacttgtcatttggcttcctttataaacattatcaactacctcagctctatcaatcacttggcag

SEQ ID NO: 506 - DNA, 120 bp, Homo sapiens
gtcatttggcttcctttataaacattatcaactacctcagctctatcaatcacttggcagtccgtagtgaacattataactcaaatgactagtcaggtctgttcattgcccatgtaaagg

SEQ ID NO: 507 - DNA, 120 bp, Homo sapiens
ctttgtcacaagtaagaaggtttaaaaatcaccataccattattggtcacaacgtttggagatagggaagagtttgtggatggatcatggcagtgcatggacagtgattagcccataaca

SEQ ID NO: 508 - DNA, 120 bp, Homo sapiens
aaagttaagggtaccttttttatatttgcatcatatctccagaccttttcctttatctccttcttgcaagttcttctttctttcagctgactatctgctgttcctgctatggctcccagt

SEQ ID NO: 509 - DNA, 120 bp, Homo sapiens
agcataaacctcagtattctttatcattcagtatcaacattattactgaaaacaataagcaatatggactgagtttctgtggggtggaaatgtgaagtggatcatagcatgatataactt

SEQ ID NO: 510 - DNA, 120 bp, Homo sapiens
gatagggaagagtttgtggatggatcatggcagtgcatggacagtgattagcccataacacaaccagtgaacactgttgtacccaaagcacataaatcaccacatatactattaatatat

SEQ ID NO: 511 - DNA, 120 bp, Homo sapiens
atagatattttgaggaagcataattttctatgtacccctcaaatcgtggctggagatgacagcctcttccacctccatataagaccatttcatttccttctacttttttctccctccttc

SEQ ID NO: 512 - DNA, 120 bp, Homo sapiens
caaccagtgaacactgttgtacccaaagcacataaatcaccacatatactattaatatatttatggatgacaacagacactataattttatgtcagtgctttctgctgtgaaaaacaaag

SEQ ID NO: 513 - DNA, 120 bp, Homo sapiens
ggcttttcaagagggtacttgttttttaagagaagacccttgaaggacagagagagcctgaatcattcaaaataatgaattactcaggatgaaatttcaataatttgcaagtgtgtggag

SEQ ID NO: 514 - DNA, 120 bp, Homo sapiens
gtgaagccgaggaattcaaggatttgagtcatgccagattgctccataaccatagcctatctttgtcacaagtaagaaggtttaaaaatcaccataccattattggtcacaacgtttgga

SEQ ID NO: 515 - DNA, 120 bp, Homo sapiens
catatacctgaagtgagaagtctgaggtaacttagcaataagcttgcagtacagtgtttagtgaagccgaggaattcaaggatttgagtcatgccagattgctccataaccatagcctat

SEQ ID NO: 516 - DNA, 120 bp, Homo sapiens
ttatggatgacaacagacactataattttatgtcagtgctttctgctgtgaaaaacaaagaaagttaagggtaccttttttatatttgcatcatatctccagaccttttcctttatctcc

SEQ ID NO: 517 - DNA, 120 bp, Homo sapiens
tccgtagtgaacattataactcaaatgactagtcaggtctgttcattgcccatgtaaaggcatatacctgaagtgagaagtctgaggtaacttagcaataagcttgcagtacagtgttta

SEQ ID NO: 518 - DNA, 120 bp, Homo sapiens
ttcttgcaagttcttctttctttcagctgactatctgctgttcctgctatggctcccagtggcttttcaagagggtacttgttttttaagagaagacccttgaaggacagagagagcctg

SEQ ID NO: 519 - DNA, 120 bp, Homo sapiens
aatcattcaaaataatgaattactcaggatgaaatttcaataatttgcaagtgtgtggagatagatattttgaggaagcataattttctatgtacccctcaaatcgtggctggagatgac

SEQ ID NO: 520 - DNA, 120 bp, Homo sapiens
caaacatagttaatgaccactccagacccagttgttatagagttggccccagctgtattgcttctatttaagactaggataagaaatgacactttcctactttttaccttattgaaaggg

SEQ ID NO: 521 - DNA, 120 bp, Homo sapiens
tgaatgaccttcaacctttcaggcttccaattttctctctgaaaaggacagccaaatgaaaactcataattttagaagatgaggttagacggttggtaggtgcatgcagagaccagttat

SEQ ID NO: 522 - DNA, 120 bp, Homo sapiens
cttctatttaagactaggataagaaatgacactttcctactttttaccttattgaaagggtagaggctcactgttatcaatctcagttcacttgttgattgcactggcttgccaagtgag

SEQ ID NO: 523 - DNA, 120 bp, Homo sapiens
aactcataattttagaagatgaggttagacggttggtaggtgcatgcagagaccagttattatttaggtattatggaagtttatagttcttgtatgttgagttcagtgtaagagtggccc

SEQ ID NO: 524 - DNA, 120 bp, Homo sapiens
tatttaggtattatggaagtttatagttcttgtatgttgagttcagtgtaagagtggccccaaacatagttaatgaccactccagacccagttgttatagagttggccccagctgtattg

SEQ ID NO: 525 - DNA, 120 bp, Homo sapiens
tcattaatgttcatttcatttatttggctatccatatgctttccagggcgaaggcaagctaggacaagggcagacaagcagccttaaagtttgggtgctttccttcgaagttgagctgcc

SEQ ID NO: 526 - DNA, 120 bp, Homo sapiens
aggacaagggcagacaagcagccttaaagtttgggtgctttccttcgaagttgagctgcctgtttgaaaatcacactttttggtgatagaagatggttccagtacagattttatttatta

SEQ ID NO: 527 - DNA, 120 bp, Homo sapiens
ctgcatctacatggatagacattttccaaagcatagctgaaaatatgtgtaagtcccagaatattttctgatttagacacagactttgagcatgataaccacatttagcatgttaggaaa

SEQ ID NO: 528 - DNA, 120 bp, Homo sapiens
gtttgcctaaatgtgtgcatccttgctggaagccagccatgattcatgctgcataagtattcattaatgttcatttcatttatttggctatccatatgctttccagggcgaaggcaagct

SEQ ID NO: 529 - DNA, 120 bp, Homo sapiens
tgtttgaaaatcacactttttggtgatagaagatggttccagtacagattttatttattactgcatctacatggatagacattttccaaagcatagctgaaaatatgtgtaagtcccaga

SEQ ID NO: 530 - DNA, 120 bp, Homo sapiens
cacactggaccagccaggaggcaggaaaatcagctggggaatgtggtgccaacgtgtgatgtttgcctaaatgtgtgcatccttgctggaagccagccatgattcatgctgcataagtat

SEQ ID NO: 531 - DNA, 120 bp, Homo sapiens
atattttctgatttagacacagactttgagcatgataaccacatttagcatgttaggaaattctgtcagaatgcttctggaaaggctacctttccagaatgaaatgaaaaaagaaaagga

SEQ ID NO: 532 - DNA, 120 bp, Homo sapiens
gcctgtttttattggaagagcaagagagagagggaatgctagctggcaatttccccaggtaccctttatgaaagtgcccttggctcttccaatttcatctgaataaccagctcaggcaaa

SEQ ID NO: 533 - DNA, 120 bp, Homo sapiens
tctcaaggtatcccaaagcactttgtaaggaaatatgacaagcgctgaggccatgcaggccagtacaacagccgccacccagcacttcacaattagtcatgcccagcctgggatcatcaa

SEQ ID NO: 534 - DNA, 120 bp, Homo sapiens
ttgtcctgggagcagatttctcaagctgcccatgtccccacactgtttgattaaaaggaggtgcttcaaactctttggctttatatagactagaatcagaatgattggtggtgcctctgt

SEQ ID NO: 535 - DNA, 120 bp, Homo sapiens
accctttatgaaagtgcccttggctcttccaatttcatctgaataaccagctcaggcaaattttcctctatcaaaaagcagaatgtgatagtgacaagctgatgcccggctgatgcccca

SEQ ID NO: 536 - DNA, 120 bp, Homo sapiens
ccttttgttctttttctgtgtttctttccccattgccatctgcagagtgttctcagtcagaagtcagctgtggggtggacagtttgtcattttaagatcatccctattctgtctaccttt

SEQ ID NO: 537 - DNA, 120 bp, Homo sapiens
tgctccatgttgaaatgcactgctcttgcaaggactgattatctatttttctgtcttccaaggccccctgtgttccactccaccctcccaattctgggggcttccaaagtgggcaggtac

SEQ ID NO: 538 - DNA, 120 bp, Homo sapiens
ctctttgcacacactccatatgaaggcaaactccagatcttggagcccatgtgtgtgtcatgcattgtactgcttcttgtacccaaatccatctcaagggtgagtagaccaggctcagac

SEQ ID NO: 539 - DNA, 120 bp, Homo sapiens
ccacaccaggaatttagaagctactcagtaaatattgcttctctcctttccccttccccagtcctgtcccccgagacattcagtagttattcacaggcatgcattctgaagtctgcctac

SEQ ID NO: 540 - DNA, 120 bp, Homo sapiens
tgcattgtactgcttcttgtacccaaatccatctcaagggtgagtagaccaggctcagacttgtcctgggagcagatttctcaagctgcccatgtccccacactgtttgattaaaaggag

SEQ ID NO: 541 - DNA, 120 bp, Homo sapiens
ttttcctctatcaaaaagcagaatgtgatagtgacaagctgatgcccggctgatgccccaggacattgactaaatagacttggcctcacaattggtttttattctctatctcctttcttc

SEQ ID NO: 542 - DNA, 120 bp, Homo sapiens
gtgcttcaaactctttggctttatatagactagaatcagaatgattggtggtgcctctgttctcaaggtatcccaaagcactttgtaaggaaatatgacaagcgctgaggccatgcaggc

SEQ ID NO: 543 - DNA, 120 bp, Homo sapiens
gtcctgtcccccgagacattcagtagttattcacaggcatgcattctgaagtctgcctactgctccatgttgaaatgcactgctcttgcaaggactgattatctatttttctgtcttcca

SEQ ID NO: 544 - DNA, 120 bp, Homo sapiens
aagtcagctgtggggtggacagtttgtcattttaagatcatccctattctgtctacctttcttatccctcatatcattgcttttagagcaaggacaattctggaagtgaaactacaataa

SEQ ID NO: 545 - DNA, 120 bp, Homo sapiens
aggccccctgtgttccactccaccctcccaattctgggggcttccaaagtgggcaggtacagaatgttctgtggagcatcggaggctgttactcaatatcttggccagcactctcaactg

SEQ ID NO: 546 - DNA, 120 bp, Homo sapiens
ggacattgactaaatagacttggcctcacaattggtttttattctctatctcctttcttcccttttgttctttttctgtgtttctttccccattgccatctgcagagtgttctcagtcag

SEQ ID NO: 547 - DNA, 120 bp, Homo sapiens
cagtacaacagccgccacccagcacttcacaattagtcatgcccagcctgggatcatcaagcctgtttttattggaagagcaagagagagagggaatgctagctggcaatttccccaggt

SEQ ID NO: 548 - DNA, 120 bp, Homo sapiens
agaatgttctgtggagcatcggaggctgttactcaatatcttggccagcactctcaactgctctttgcacacactccatatgaaggcaaactccagatcttggagcccatgtgtgtgtca

SEQ ID NO: 549 - DNA, 120 bp, Homo sapiens
attttatatatcccaatagagaagcgtggaagacatctaggttgccactgtcatttgaaattggaatttttaaaagagaaacctgaagacttgaagaaagctttcttttgcctcccctta

SEQ ID NO: 550 - DNA, 120 bp, Homo sapiens
aaagaagtccactgttagtatcttttcccctgcctagtttgtaagcaactggcctcttctatttgtaagttacctgttttcatttccatatgccccaaagcaaactttagctcacggcct

SEQ ID NO: 551 - DNA, 120 bp, Homo sapiens
atttgtaagttacctgttttcatttccatatgccccaaagcaaactttagctcacggccttacagagtgtgtatgttagtatgttaaaatgaaatcaactttcctctcccaggccttcta

SEQ ID NO: 552 - DNA, 120 bp, Homo sapiens
caaggggaggagctgtatactcgagcatgccctgtggttcctggccctgactgagggactattttatatatcccaatagagaagcgtggaagacatctaggttgccactgtcatttgaaa

SEQ ID NO: 553 - DNA, 120 bp, Homo sapiens
tacagagtgtgtatgttagtatgttaaaatgaaatcaactttcctctcccaggccttctaattgacatgaatttgggagtagacttgcattggcctttgtcctgacagccaacagagtcc

SEQ ID NO: 554 - DNA, 120 bp, Homo sapiens
cagccagtttcccctctgttttctcccttgcttacacagccaaggagtggagccaagcctcaaggggaggagctgtatactcgagcatgccctgtggttcctggccctgactgagggact

SEQ ID NO: 555 - DNA, 120 bp, Homo sapiens
gcttcaacattgctcaccctgaccaggtatgaagccaagagtttggtttagggcataaaagaatgtcggaactcaaggactaggttgaggtggggaagggggatgaaggcttcttttttt

SEQ ID NO: 556 - DNA, 120 bp, Homo sapiens
tatacattttgagggcagactcaacttgagtaaacctgattgagctttccccatctgcctcccagagatcactgcctgtgctttgttaaaaagagaattataggagtcctctcaaggcag

SEQ ID NO: 557 - DNA, 120 bp, Homo sapiens
attgacatgaatttgggagtagacttgcattggcctttgtcctgacagccaacagagtcctcttctgttgtattcactgttgccttccatgaggatcccatggagaaagtttgtcattga

SEQ ID NO: 558 - DNA, 120 bp, Homo sapiens
tcttctgttgtattcactgttgccttccatgaggatcccatggagaaagtttgtcattgatatacattttgagggcagactcaacttgagtaaacctgattgagctttccccatctgcct

SEQ ID NO: 559 - DNA, 120 bp, Homo sapiens
ttggaatttttaaaagagaaacctgaagacttgaagaaagctttcttttgcctccccttacagttgatttttgagcttcttaaagctacctagtccaaagtacccacactcttattcttt

SEQ ID NO: 560 - DNA, 120 bp, Homo sapiens
cccagagatcactgcctgtgctttgttaaaaagagaattataggagtcctctcaaggcagagaggcctaaaattagacatggcagccatgcctttggtgtgcatggaggttggatacagg

SEQ ID NO: 561 - DNA, 120 bp, Homo sapiens
cagttgatttttgagcttcttaaagctacctagtccaaagtacccacactcttattcttttgtctttcctactggttttatttttttttcatcttcccaggtgtttgatgatcactaaga

SEQ ID NO: 562 - DNA, 120 bp, Homo sapiens
tgtctttcctactggttttatttttttttcatcttcccaggtgtttgatgatcactaagagcttcaacattgctcaccctgaccaggtatgaagccaagagtttggtttagggcataaaa

SEQ ID NO: 563 - DNA, 120 bp, Homo sapiens
gaatgtcggaactcaaggactaggttgaggtggggaagggggatgaaggcttctttttttcttgggttaagcagaaataacttagatctcagagtgaaagccttgaattatcacatatat

SEQ ID NO: 564 - DNA, 120 bp, Homo sapiens
agaggcctaaaattagacatggcagccatgcctttggtgtgcatggaggttggatacaggcagccagtttcccctctgttttctcccttgcttacacagccaaggagtggagccaagcct

SEQ ID NO: 565 - DNA, 120 bp, Homo sapiens
cttgggttaagcagaaataacttagatctcagagtgaaagccttgaattatcacatatatcactggaaaagactagttctttgctatgataacaattgttcatcatctctcccctgagga

SEQ ID NO: 566 - DNA, 120 bp, Homo sapiens
gggctagaagtaagcatgctactagaaacagaatttgggaacacagctctgggcctagaaaagcgacctgtcaacttgttacagttaacatcaataactataggatgggtttggtggaaa

SEQ ID NO: 567 - DNA, 120 bp, Homo sapiens
aaattggctgcgttctgaatcctatttttatttgggataacaataagcctgtatggtcactgtgacctttgatttgctgtttctgcaacctcacacttgtctcaggattcttcttccact

SEQ ID NO: 568 - DNA, 120 bp, Homo sapiens
tgtgacctttgatttgctgtttctgcaacctcacacttgtctcaggattcttcttccacttctgcactttatattgggtttcttccaggcatcatattaaactttaagccaggtatgtgt

SEQ ID NO: 569 - DNA, 120 bp, Homo sapiens
tctgcactttatattgggtttcttccaggcatcatattaaactttaagccaggtatgtgtatatgcatgggctgtgggcctgaaaaaaattagcccgagagagaaaaaaatttaagtagt

SEQ ID NO: 570 - DNA, 120 bp, Homo sapiens
atatgcatgggctgtgggcctgaaaaaaattagcccgagagagaaaaaaatttaagtagtgggctagaagtaagcatgctactagaaacagaatttgggaacacagctctgggcctagaa

SEQ ID NO: 571 - DNA, 120 bp, Homo sapiens
gtaaactatactttctcctttccagactatcctagtaagaaaattctcttttaagacagagtagaactctggaattcatcagttttgatgtttcttaaagtgtaatctaagatagtgctc

SEQ ID NO: 572 - DNA, 120 bp, Homo sapiens
ctgtattaagttctgatgtctgaccattgttcaaataaagagtaaaatgcaaatgacaggaaattggctgcgttctgaatcctatttttatttgggataacaataagcctgtatggtcac

SEQ ID NO: 573 - DNA, 120 bp, Homo sapiens
gtagaactctggaattcatcagttttgatgtttcttaaagtgtaatctaagatagtgctcctgtattaagttctgatgtctgaccattgttcaaataaagagtaaaatgcaaatgacagg

SEQ ID NO: 574 - DNA, 120 bp, Homo sapiens
aagcgacctgtcaacttgttacagttaacatcaataactataggatgggtttggtggaaaattatgctgaccaacagggtgggagagaatagggtcagaatatatatcgctgtaaggttg

SEQ ID NO: 575 - DNA, 120 bp, Homo sapiens
aactggagagcttgacttctcttgaatatatttttaaataaagtactcctttcaactccaaatgcagcaggcttggttcccttctcctacctccattgcggatgaaagcttaatctttaa

SEQ ID NO: 576 - DNA, 120 bp, Homo sapiens
gttgcagcactgtcattctccagtttctcttggagttgtcaccaccctctcctttgttctcactgctgacatcatttgtaaaataatttcttcccttaaataaacaagacatacaatcct

SEQ ID NO: 577 - DNA, 120 bp, Homo sapiens
taaggcccaacagggctctccaaggaggcaaaattctgatgatacatttctgtttagtggaaaatgggtagggaaaattatgtcttagaatcaattaaccaaacataaaatcctccaagg

SEQ ID NO: 578 - DNA, 120 bp, Homo sapiens
aatgcagcaggcttggttcccttctcctacctccattgcggatgaaagcttaatctttaagatgggcttgggtgggtagagtacgccccttggtgagcactgtgctctctgcaaccccaa

SEQ ID NO: 579 - DNA, 120 bp, Homo sapiens
atttaaaatatgatttgattatgtacatttcagatttttctacctttctaggagtatctctgttgtataaaaacacaaaattctggaacttttgaaaggaagatgtgcctctcttcatac

SEQ ID NO: 580 - DNA, 120 bp, Homo sapiens
cactgctgacatcatttgtaaaataatttcttcccttaaataaacaagacatacaatcctctaaatgactaaagaacagttacctagaagaaaccttagtggaaagtattttcttcatct

SEQ ID NO: 581 - DNA, 120 bp, Homo sapiens
tttcacttcctaaaaagattttgaataagatgtcttttaagtaagaagctccctgaatgcatttaaaatatgatttgattatgtacatttcagatttttctacctttctaggagtatctc

SEQ ID NO: 582 - DNA, 120 bp, Homo sapiens
gcacaatatagggcaatccaggtttacacaaaggattaatttgggaacaattatcctcattttcacttcctaaaaagattttgaataagatgtcttttaagtaagaagctccctgaatgc

SEQ ID NO: 583 - DNA, 120 bp, Homo sapiens
tgtggggatgtgaggatctatgtctaccaattgcagcctctgctgcaaattggaggcagaaatctgggctgaacaataggtaagagtgtcaactctacagatctctcacatgctaagcaa

SEQ ID NO: 584 - DNA, 120 bp, Homo sapiens
aatctgggctgaacaataggtaagagtgtcaactctacagatctctcacatgctaagcaagcacaatatagggcaatccaggtttacacaaaggattaatttgggaacaattatcctcat

SEQ ID NO: 585 - DNA, 120 bp, Homo sapiens
ggcctcttaataaagaacttgtggtttgagtgttcattgaaattagccatattaggtttatgtggggatgtgaggatctatgtctaccaattgcagcctctgctgcaaattggaggcaga

SEQ ID NO: 586 - DNA, 120 bp, Homo sapiens
ggcttggtaggatgcctagggaagagccacgagataaaaactccaggctggaagggcattgttgcagcactgtcattctccagtttctcttggagttgtcaccaccctctcctttgttct

SEQ ID NO: 587 - DNA, 120 bp, Homo sapiens
aagagatgcatgctgacttaaaaggcatgatatatgtgaaactaagataatgtgttcaagagtgatgctttgttgatgcagaaccactgaattccttactattatgtttgcctgactatc

SEQ ID NO: 588 - DNA, 120 bp, Homo sapiens
tgttgtataaaaacacaaaattctggaacttttgaaaggaagatgtgcctctcttcatacatttgtcattcttgaacgattgtaaaatgaagtgactgcatatcacgtcatgtgccctat

SEQ ID NO: 589 - DNA, 120 bp, Homo sapiens
ctaaatgactaaagaacagttacctagaagaaaccttagtggaaagtattttcttcatctaacggatgattgtctttacagaggtggagtaaaggatgtgcgagggagcataatcaagct

SEQ ID NO: 590 - DNA, 120 bp, Homo sapiens
aacggatgattgtctttacagaggtggagtaaaggatgtgcgagggagcataatcaagctaagagatgcatgctgacttaaaaggcatgatatatgtgaaactaagataatgtgttcaag

SEQ ID NO: 591 - DNA, 120 bp, Homo sapiens
gatgggcttgggtgggtagagtacgccccttggtgagcactgtgctctctgcaaccccaataaggcccaacagggctctccaaggaggcaaaattctgatgatacatttctgtttagtgg

SEQ ID NO: 592 - DNA, 120 bp, Homo sapiens
aaaatgggtagggaaaattatgtcttagaatcaattaaccaaacataaaatcctccaaggggcttggtaggatgcctagggaagagccacgagataaaaactccaggctggaagggcatt

SEQ ID NO: 593 - DNA, 120 bp, Homo sapiens
agtgatgctttgttgatgcagaaccactgaattccttactattatgtttgcctgactatcggcctcttaataaagaacttgtggtttgagtgttcattgaaattagccatattaggttta

SEQ ID NO: 594 - DNA, 120 bp, Homo sapiens
taagttatgtcaatatctatttataaagatttttgtgatattcttttcactgtagaacttcaagcatatcctaaaaggaacggttagatacctctacaaactgtggcaatgacttactga

SEQ ID NO: 595 - DNA, 120 bp, Homo sapiens
gtttcagaagagcaaaactaagacaatccagggaaatgccatttgagaatttctaactttaaaaaaacaagtaaaatagtgccaagaatattatctaactaaccccaaagtctacaatgt

SEQ ID NO: 596 - DNA, 120 bp, Homo sapiens
gtaattgctggcaactgatttttggtgcttcttgttttgatagtatagcagtgcgagtaggtttcagaagagcaaaactaagacaatccagggaaatgccatttgagaatttctaacttt

SEQ ID NO: 597 - DNA, 120 bp, Homo sapiens
aactcttttattttgataatgctgttctaaccctatctacttcagtcctttcccacccagctggtttaggaatcaaattcccaatgtttcatcactgttaacattactgttttactcttc

SEQ ID NO: 598 - DNA, 120 bp, Homo sapiens
ctggtttaggaatcaaattcccaatgtttcatcactgttaacattactgttttactcttcactttagttcttaaatggcatagtgtcttaaattccctcagcctctttcacatttgattt

SEQ ID NO: 599 - DNA, 120 bp, Homo sapiens
aaaaaaacaagtaaaatagtgccaagaatattatctaactaaccccaaagtctacaatgtaactcttttattttgataatgctgttctaaccctatctacttcagtcctttcccacccag

SEQ ID NO: 600 - DNA, 120 bp, Homo sapiens
ctttggaaactttttaccttttcattgaagcccatatgatcttttccgaaacagacccttatctttacctccttctttggagtctttctcctacttgaatttctgaacttcttaaaatgg

SEQ ID NO: 601 - DNA, 120 bp, Homo sapiens
actttagttcttaaatggcatagtgtcttaaattccctcagcctctttcacatttgatttctttggaaactttttaccttttcattgaagcccatatgatcttttccgaaacagaccctt

SEQ ID NO: 602 - DNA, 120 bp, Homo sapiens
caagcatatcctaaaaggaacggttagatacctctacaaactgtggcaatgacttactgagtaattgctggcaactgatttttggtgcttcttgttttgatagtatagcagtgcgagtag

SEQ ID NO: 603 - DNA, 120 bp, Homo sapiens
actattttgtcatacaggcaacagaatgttaaaccatttcataaaacaatgacaaatatacatgaatttttcatcagttataaatgcatttcctttataacattgaacatgtttttgcaa

SEQ ID NO: 604 - DNA, 120 bp, Homo sapiens
ttaattttttcagattaatttttcagaaaagtgactgtttctgtctattgtcttaaccccaggcatcaaaggattttaatcagaaagaaccgaggaataatttggttattttagtgcctt

SEQ ID NO: 605 - DNA, 120 bp, Homo sapiens
catgaatttttcatcagttataaatgcatttcctttataacattgaacatgtttttgcaactgaaataagtacggttttcatttttagaaggcacatgataaagttaaggcagtggttaa

SEQ ID NO: 606 - DNA, 120 bp, Homo sapiens
ctgaaataagtacggttttcatttttagaaggcacatgataaagttaaggcagtggttaattaattttttcagattaatttttcagaaaagtgactgtttctgtctattgtcttaacccc

SEQ ID NO: 607 - DNA, 120 bp, Homo sapiens
ccagtaataagttttcttaagtgctttcttaatattctgatatttttaaaaaagatctggactattttgtcatacaggcaacagaatgttaaaccatttcataaaacaatgacaaatata

SEQ ID NO: 608 - DNA, 120 bp, Homo sapiens
accctattttagagctttgtcaagctttggaaagaaaaccatttataatataatagataaattatggatatttgaggcagtttttatcatagtatacatggtaaaccacagccccccttt

SEQ ID NO: 609 - DNA, 120 bp, Homo sapiens
ttacttttatcttaaacatgttttaacaaagcaagcatatgtagattagcactaattaaaacaaaaacctttgtaatgatagctgttttttatatgattacaaaaaatttactatacaaa

SEQ ID NO: 610 - DNA, 120 bp, Homo sapiens
actgtatattgacaaatttagcaacaaaatgagcttgagaaaaaaatcaaggcctgccatggcatctttgcttttttttcttaaaaaaaaaactttttagaaagattatgcgactgtatt

SEQ ID NO: 611 - DNA, 120 bp, Homo sapiens
ggcatctttgcttttttttcttaaaaaaaaaactttttagaaagattatgcgactgtattatctgtaactactgcaatggtgtaaatcctgatggtataatttgctttttaaagctatct

SEQ ID NO: 612 - DNA, 120 bp, Homo sapiens
gatgagagctgtgtcacagctaatttttctttagtaattaaaggtttataaaaatcttacactgtatattgacaaatttagcaacaaaatgagcttgagaaaaaaatcaaggcctgccat

SEQ ID NO: 613 - DNA, 120 bp, Homo sapiens
ggaagacagttaccatttcagatcggcagaagttgtggctttaatctagactcgaatatgttttacatcaaagggttgcctcaacagtgctcaaacctgcctctctgaaaacatgctgag

SEQ ID NO: 614 - DNA, 120 bp, Homo sapiens
ttttacatcaaagggttgcctcaacagtgctcaaacctgcctctctgaaaacatgctgagcacgaaggttacttgaagtcttagcttgagtacttaagagagtgctatggagggattgtt

SEQ ID NO: 615 - DNA, 120 bp, Homo sapiens
cacgaaggttacttgaagtcttagcttgagtacttaagagagtgctatggagggattgttgatgagagctgtgtcacagctaatttttctttagtaattaaaggtttataaaaatcttac

SEQ ID NO: 616 - DNA, 120 bp, Homo sapiens
gctcctccgctttttcagttcctaaataattttccaatctagaatgcaaaagattctgaaggaagacagttaccatttcagatcggcagaagttgtggctttaatctagactcgaatatg

SEQ ID NO: 617 - DNA, 120 bp, Homo sapiens
tctatcttggcctccatatgcaatcaaataagaaacacattttaagcatattatttaccttgtggattctgccttcctcagtgtgttcagtctgtgtatattcatttctcccacactgta

SEQ ID NO: 618 - DNA, 120 bp, Homo sapiens
cattttaaagtctcattttagattattttgactatctgatttttaaaatggtttaaaaaatctatcttggcctccatatgcaatcaaataagaaacacattttaagcatattatttacct

SEQ ID NO: 619 - DNA, 120 bp, Homo sapiens
aagcatacatagactagtgagcaccactcattacatgtcatttctctagagaaactagttgggccatggctgcaggactctcacttgaaaagacatgtgtggtgatgttttctcaggcag

SEQ ID NO: 620 - DNA, 120 bp, Homo sapiens
ttaagcaataaagtgtaccctgatttgcactgaaaataaagattcctttaaagggagcagttctagttatctctctctttaggtaccatatgctgaacgtttttctatgcactaaaacag

SEQ ID NO: 621 - DNA, 120 bp, Homo sapiens
ttatttcctttgtttttcaattgatatttactcttaaattaaactaattatttaaaaaagcattttaaagtctcattttagattattttgactatctgatttttaaaatggtttaaaaaa

SEQ ID NO: 622 - DNA, 120 bp, Homo sapiens
agggtcttcttggtgactccacttcttttctttgtttatgtatccttccagatgatgtatttatttcctttgtttttcaattgatatttactcttaaattaaactaattatttaaaaaag

SEQ ID NO: 623 - DNA, 120 bp, Homo sapiens
tgtggattctgccttcctcagtgtgttcagtctgtgtatattcatttctcccacactgtaagaagctagtcagatgtataattggattatcatgctacataatcttagcacactcatttt

SEQ ID NO: 624 - DNA, 120 bp, Homo sapiens
gggccatggctgcaggactctcacttgaaaagacatgtgtggtgatgttttctcaggcagttaagcaataaagtgtaccctgatttgcactgaaaataaagattcctttaaagggagcag

SEQ ID NO: 625 - DNA, 120 bp, Homo sapiens
agaagctagtcagatgtataattggattatcatgctacataatcttagcacactcattttaagcatacatagactagtgagcaccactcattacatgtcatttctctagagaaactagtt

SEQ ID NO: 626 - DNA, 120 bp, Homo sapiens
tctagccccacctttgggccccttcataagcaaacatgcaggttttccagagagctgtatgctactgaatgcagaaaatttggctcatactggcctatggactatctgctcactgccctg

SEQ ID NO: 627 - DNA, 120 bp, Homo sapiens
agctcttgaactgagctctttaactgacctcagctcttgaactatggtacaagatcccatggtcctgtttggtacctccatttgccctccttttcactctctgggagcatagctaagttc

SEQ ID NO: 628 - DNA, 120 bp, Homo sapiens
ctggaaaacgaaaaatagaattatccaactaccactttaaaattggacaaagacttttgttgttgttgttggagggggtggtaaacatcattttagcagaccaaatatacttttggtgaa

SEQ ID NO: 629 - DNA, 120 bp, Homo sapiens
gctactgaatgcagaaaatttggctcatactggcctatggactatctgctcactgccctgataactattttccaagggagtgggtgccctacctttcctacatgaagttttttgctagtc

SEQ ID NO: 630 - DNA, 120 bp, Homo sapiens
ttgccctaaaaattctaggtatcccttgcttttaggataaatatgtttcactgggaccagctggaaaacgaaaaatagaattatccaactaccactttaaaattggacaaagacttttgt

SEQ ID NO: 631 - DNA, 120 bp, Homo sapiens
ataactattttccaagggagtgggtgccctacctttcctacatgaagttttttgctagtcttgccctaaaaattctaggtatcccttgcttttaggataaatatgtttcactgggaccag

SEQ ID NO: 632 - DNA, 120 bp, Homo sapiens
ggtcctgtttggtacctccatttgccctccttttcactctctgggagcatagctaagttcaaaattgaattaggtacttgtagtaagagcatacttataatcctgggatcttcatgttgc

SEQ ID NO: 633 - DNA, 120 bp, Homo sapiens
cagatattaacctcttgaagtttttcaccacaacctgggcacttttctgatttgctcacttctagccccacctttgggccccttcataagcaaacatgcaggttttccagagagctgtat

SEQ ID NO: 634 - DNA, 120 bp, Homo sapiens
aaaattgaattaggtacttgtagtaagagcatacttataatcctgggatcttcatgttgccagatattaacctcttgaagtttttcaccacaacctgggcacttttctgatttgctcact

SEQ ID NO: 635 - DNA, 120 bp, Homo sapiens
cactgctccttatgaaaatgaggaaataattgagatgagtgagccattgaggcaacagtacaaaaagtgctgaaaactcactgcttaaataagcacctcttactgcttttgtggcacttt

SEQ ID NO: 636 - DNA, 120 bp, Homo sapiens
gagatgagaagtaggaggtggagagaactaaccagaagagggtacccaaataaaccagaaatatgtatgtgttagagaaggggcctattgagcgggtggcagtggcatgtgtggcattac

SEQ ID NO: 637 - DNA, 120 bp, Homo sapiens
attctgactagaaaagtaatgtttggttttgtttggctctttgcttaatatacccataataagggtacctatttgcctttggaccattagttcaaatattattttattaatatggaatta

SEQ ID NO: 638 - DNA, 120 bp, Homo sapiens
ctgggctccagaagccatagtcttcttagctgctccctatccccactctcacctcaatttttttttttcacttttgtttttcttctcagggaaaggtttgaggcaaagaatgtcttctta

SEQ ID NO: 639 - DNA, 120 bp, Homo sapiens
atggacatggtagaatctttgtcctgagggagctatctagatccattccttctgatatgcagccagtagccacttgtggtaatggagcaatagaaacaacactagttcaagtggaaacgt

SEQ ID NO: 640 - DNA, 120 bp, Homo sapiens
atatgtatgtgttagagaaggggcctattgagcgggtggcagtggcatgtgtggcattacttgctcctgtattctctgctttttacttagttgtggctttggtggtatagtctcaaatct

SEQ ID NO: 641 - DNA, 120 bp, Homo sapiens
tgatccaaaaccaagcatggtggtgatttattcaccaagagattcctaagtacctgtgtgatggacatggtagaatctttgtcctgagggagctatctagatccattccttctgatatgc

SEQ ID NO: 642 - DNA, 120 bp, Homo sapiens
aagttacgtaggtaatattgttatgtatcatgttttggcaatgtagactaaatacttgctcataagagtacaggacaatgaggatagtttggttttgtttactgcatggaaaatgcagga

SEQ ID NO: 643 - DNA, 120 bp, Homo sapiens
aagggtacctatttgcctttggaccattagttcaaatattattttattaatatggaattactgggctccagaagccatagtcttcttagctgctccctatccccactctcacctcaattt

SEQ ID NO: 644 - DNA, 120 bp, Homo sapiens
agccagtagccacttgtggtaatggagcaatagaaacaacactagttcaagtggaaacgtgagatgagaagtaggaggtggagagaactaaccagaagagggtacccaaataaaccagaa

SEQ ID NO: 645 - DNA, 120 bp, Homo sapiens
ttttttttcacttttgtttttcttctcagggaaaggtttgaggcaaagaatgtcttcttatgatccaaaaccaagcatggtggtgatttattcaccaagagattcctaagtacctgtgtg

SEQ ID NO: 646 - DNA, 120 bp, Homo sapiens
cataagagtacaggacaatgaggatagtttggttttgtttactgcatggaaaatgcaggatgtttagtaaatagattcatggcgtagtgagttcactactaaaatcagactctgagaatg

SEQ ID NO: 647 - DNA, 120 bp, Homo sapiens
ttgctcctgtattctctgctttttacttagttgtggctttggtggtatagtctcaaatctaagttacgtaggtaatattgttatgtatcatgttttggcaatgtagactaaatacttgct

SEQ ID NO: 648 - DNA, 120 bp, Homo sapiens
tgtttagtaaatagattcatggcgtagtgagttcactactaaaatcagactctgagaatgggtttgatttaaatggctagtttagaagactgaatttaggccacttgattgagaaaggcc

SEQ ID NO: 649 - DNA, 120 bp, Homo sapiens
aacaaatgaatacaattttagagtaaatttttactgcttacactacatgtagattttctttttagagatttcgcaatgctgatttatttcaaaataagcttgaagctaagcgacaaagct

SEQ ID NO: 650 - DNA, 120 bp, Homo sapiens
tgctcaatcaatgttgattctatgctaccaacaaaaatgagtccatgatgtttactattcaacaaatgaatacaattttagagtaaatttttactgcttacactacatgtagattttctt

SEQ ID NO: 651 - DNA, 120 bp, Homo sapiens
tttagagatttcgcaatgctgatttatttcaaaataagcttgaagctaagcgacaaagctgaatgatgatttgttttttatttatttttaaatccaaacttacaattttacatgtcattg

SEQ ID NO: 652 - DNA, 120 bp, Homo sapiens
aaagtacattttggcccatttactccttaaataattttatgtctcccaaggagagttgtaagttgcttgatagtaaatgctatgtattttgtaccttagtgtatatattatgggatttca

SEQ ID NO: 653 - DNA, 120 bp, Homo sapiens
ttataaacaaaataaaggtaatttggaggaaaactgtcttcagttatattggatatttgggggacatttttgtatgttagttagcaaagatcacttgaaaaagaagattcttccttctat

SEQ ID NO: 654 - DNA, 120 bp, Homo sapiens
ttaaaataaattaaatggatgatttcagccagatcattatgaaaacacatgagatattgggttatgcaatgactaacagtgtgtaccttttcttgatatttattcataaactggggaata

SEQ ID NO: 655 - DNA, 120 bp, Homo sapiens
tcacaaaattaaaaaaaactgtgcattaaagaaaaacaaaaataaaaaccatagttcaagttataaacaaaataaaggtaatttggaggaaaactgtcttcagttatattggatatttgg

SEQ ID NO: 656 - DNA, 120 bp, Homo sapiens
gttatgcaatgactaacagtgtgtaccttttcttgatatttattcataaactggggaataaaagtacattttggcccatttactccttaaataattttatgtctcccaaggagagttgta

SEQ ID NO: 657 - DNA, 120 bp, Homo sapiens
gattcaagggagcctagcaaaaaataaatgaaatgaaataaaataatacaaagagaaaagattattccataaattctgcttacttatttctggcaaacttgttgacagcacatgtgacct

SEQ ID NO: 658 - DNA, 120 bp, Homo sapiens
attattccataaattctgcttacttatttctggcaaacttgttgacagcacatgtgaccttttggtaaaaagacatttttatatttttagttaagtttcaaatataaattgtttgtgttt

SEQ ID NO: 659 - DNA, 120 bp, Homo sapiens
tttggtaaaaagacatttttatatttttagttaagtttcaaatataaattgtttgtgtttttaaaataaattaaatggatgatttcagccagatcattatgaaaacacatgagatattgg

SEQ ID NO: 660 - DNA, 120 bp, Homo sapiens
gggacatttttgtatgttagttagcaaagatcacttgaaaaagaagattcttccttctatgattcaagggagcctagcaaaaaataaatgaaatgaaataaaataatacaaagagaaaag

SEQ ID NO: 661 - DNA, 120 bp, Homo sapiens
acactcaataatgtagttagtgaattatttagaaagaggcattttgagcccataatgtatgataggtacttctacatttattattttattctttgcagacctgcagaaaactgtaagaaa

SEQ ID NO: 662 - DNA, 120 bp, Homo sapiens
gataggtacttctacatttattattttattctttgcagacctgcagaaaactgtaagaaaaaagtttatttcagattcatgtgtttatttgattaatctcttcataggtttcatttttca

SEQ ID NO: 663 - DNA, 120 bp, Homo sapiens
aaagtttatttcagattcatgtgtttatttgattaatctcttcataggtttcatttttcagctcctgtcagaaaatacagattcttataaggttcaccttttacccataagaataatagt

SEQ ID NO: 664 - DNA, 120 bp, Homo sapiens
tagaaaggataattcttatttcatatttatctttttgtttcagaataataaactaagctatctctactcagtccattttaatacaaaaatatttttacccggactgagtttttatgcttt

SEQ ID NO: 665 - DNA, 120 bp, Homo sapiens
accataacctctttataaaaattttctggaaagtttacatgacagtaagtaatatataattagaaaggataattcttatttcatatttatctttttgtttcagaataataaactaagcta

SEQ ID NO: 666 - DNA, 120 bp, Homo sapiens
atagcttgcccacagtaatgtgtcctgaaaaatatgacaattaattaagttggagacagaaccataacctctttataaaaattttctggaaagtttacatgacagtaagtaatatataat

SEQ ID NO: 667 - DNA, 120 bp, Homo sapiens
ccattttagtgagattcctgtgctgatagtcatacccatatcaaatcctctttggcagttatagcttgcccacagtaatgtgtcctgaaaaatatgacaattaattaagttggagacaga

SEQ ID NO: 668 - DNA, 120 bp, Homo sapiens
tctctactcagtccattttaatacaaaaatatttttacccggactgagtttttatgctttttaggaactttgtatctgcctcacttagttaaaatcctagctgcactaatcacttactgt

SEQ ID NO: 669 - DNA, 120 bp, Homo sapiens
aaattgtctctaggtagactttcccacaatgcaattttaggatacagaggtcatatgcctgttattctactgtggcagagaaaatatggagcctggaaaactgttcatttgcatcacata

SEQ ID NO: 670 - DNA, 120 bp, Homo sapiens
tactgattctcccaggcctatacaaatcctttgatacacaaatgaatagtaaaggaacataaattgtctctaggtagactttcccacaatgcaattttaggatacagaggtcatatgcct

SEQ ID NO: 671 - DNA, 120 bp, Homo sapiens
caataaaaaactaatatatttaccatgcaaggcaaggcatttatcctctcatgattcagtttctttttacctgacataatggaattaatttatactgctgtgaagttgtagttgagaaac

SEQ ID NO: 672 - DNA, 120 bp, Homo sapiens
atgacttctaaagtaatagaggacatgtattattaattttagtagtattaatagtaatgatactgattctcccaggcctatacaaatcctttgatacacaaatgaatagtaaaggaacat

SEQ ID NO: 673 - DNA, 120 bp, Homo sapiens
ttctttttacctgacataatggaattaatttatactgctgtgaagttgtagttgagaaacatgacttctaaagtaatagaggacatgtattattaattttagtagtattaatagtaatga

SEQ ID NO: 674 - DNA, 120 bp, Homo sapiens
agagatgatttagaagagttgttttggaaaaggagaagacagaaaatgttttagaggtgtcatagagataaaattggcatggcatggtgcaaggaggtaaagcccaatagctttgtaagg

SEQ ID NO: 675 - DNA, 120 bp, Homo sapiens
tgagagggtgggtaacagcaatagtcagaggaaagaacccttttatacatgatggtacaggaacaacactggcttccaaccccacagctgctctttaacagaaggtcagaagctggggag

SEQ ID NO: 676 - DNA, 120 bp, Homo sapiens
tcacccgttccaagaggccttaaaggtgttgatctgttccctgggcatcaccacattccacaaattaatgttcctctgagagaatagggtgattcaatttcactgtgcccgaaggttact

SEQ ID NO: 677 - DNA, 120 bp, Homo sapiens
tttctctaacccttagcatgtataaactgatctgttgggaaatgtgtagcatttataggatggtaggatttgtaacatgcgatcacaggactgtttatatagagtccctgggaaggggag

SEQ ID NO: 678 - DNA, 120 bp, Homo sapiens
tttggggttcatgtttgttctaagtctatgctaatgatctgccaactgtctgtttgtcactttctctaacccttagcatgtataaactgatctgttgggaaatgtgtagcatttatagga

SEQ ID NO: 679 - DNA, 120 bp, Homo sapiens
caaattaatgttcctctgagagaatagggtgattcaatttcactgtgcccgaaggttacttttggggttcatgtttgttctaagtctatgctaatgatctgccaactgtctgtttgtcac

SEQ ID NO: 680 - DNA, 120 bp, Homo sapiens
ctaaggatgggaagtagattagttgaggccactgcagtggggtctgcaagttgctagcactcacccgttccaagaggccttaaaggtgttgatctgttccctgggcatcaccacattcca

SEQ ID NO: 681 - DNA, 120 bp, Homo sapiens
acagaggacacagttggcatttccttttggtgttgaggggagatgtgtacatggttgtgagatgactcaccctttttgcttagatagttccactttcattgtggacagactctttggagg

SEQ ID NO: 682 - DNA, 120 bp, Homo sapiens
tcaaataaattagtcattggtgccagagtatcaaataattatggtacagaatgtatttctctgaatgacaccttctcccagagattctgatatatattcctctgcactcaccctgtttga

SEQ ID NO: 683 - DNA, 120 bp, Homo sapiens
ctcagggaccagtgtgagaatgggaactttatgatctggagctggttaagtgaagtccaaaaataattaagaaagtgtttccttccctgggaatgagttcagtaggaatctcaatgtatt

SEQ ID NO: 684 - DNA, 120 bp, Homo sapiens
aaataattaagaaagtgtttccttccctgggaatgagttcagtaggaatctcaatgtattgtagagcactaaggactcagcctcaggcatttgcaaaggattcttccagttgcctgtgtt

SEQ ID NO: 685 - DNA, 120 bp, Homo sapiens
atttgttgagtggtttgccattttgttttacagccactctgtgggctatgaaatggtcatccggccgctttatttgtccctaaaaaaagcagtttttccctttcttatcttcatggctgc

SEQ ID NO: 686 - DNA, 120 bp, Homo sapiens
ctgaatgacaccttctcccagagattctgatatatattcctctgcactcaccctgtttgataattaccagtatatggaccatttacctgaagaataagagtagggtttcctactgttgtt

SEQ ID NO: 687 - DNA, 120 bp, Homo sapiens
gcacatatccattttcaataaacatgaaagtttcataccctcttttaatgtttgaaatcctcaaataaattagtcattggtgccagagtatcaaataattatggtacagaatgtatttct

SEQ ID NO: 688 - DNA, 120 bp, Homo sapiens
gtagagcactaaggactcagcctcaggcatttgcaaaggattcttccagttgcctgtgttacagaggacacagttggcatttccttttggtgttgaggggagatgtgtacatggttgtga

SEQ ID NO: 689 - DNA, 120 bp, Homo sapiens
gccagtttggcatgcacgtgtgtgttcattccatcctggagcattctttatgagaaagccatttgttgagtggtttgccattttgttttacagccactctgtgggctatgaaatggtcat

SEQ ID NO: 690 - DNA, 120 bp, Homo sapiens
taattaccagtatatggaccatttacctgaagaataagagtagggtttcctactgttgttgaaaatttgcttgactcttaacaacttgtgtgtgactgtaacaagatcacacagggtaaa

SEQ ID NO: 691 - DNA, 120 bp, Homo sapiens
gaaaatttgcttgactcttaacaacttgtgtgtgactgtaacaagatcacacagggtaaacaatattagcttattcaaccactggctgaagaaatttaggaaagtgaacacatttttctt

SEQ ID NO: 692 - DNA, 120 bp, Homo sapiens
caatattagcttattcaaccactggctgaagaaatttaggaaagtgaacacatttttctttacatttctctttgttctgtgagccttttatgctggaatagttttcactgcaggctgtta

SEQ ID NO: 693 - DNA, 120 bp, Homo sapiens
aaactgatgatgtattggatttgataattcatcaaatctgaggtttactggtttgtatttgcctcaaaatgggcatataatattttgtcaggtaacataatagacagatcattggcattg

SEQ ID NO: 694 - DNA, 120 bp, Homo sapiens
ccggccgctttatttgtccctaaaaaaagcagtttttccctttcttatcttcatggctgccaagcagcagaaagagtaactcagggaagccatgtgatagccttttatctgtctgttcag

SEQ ID NO: 695 - DNA, 120 bp, Homo sapiens
gatgactcaccctttttgcttagatagttccactttcattgtggacagactctttggagggccagtttggcatgcacgtgtgtgttcattccatcctggagcattctttatgagaaagcc

SEQ ID NO: 696 - DNA, 120 bp, Homo sapiens
caagcagcagaaagagtaactcagggaagccatgtgatagccttttatctgtctgttcagaaactgatgatgtattggatttgataattcatcaaatctgaggtttactggtttgtattt

SEQ ID NO: 697 - DNA, 120 bp, Homo sapiens
acctctttccaaggttagttgccaatggcatctttggaacagtgtccttcacttttgtccctcagggaccagtgtgagaatgggaactttatgatctggagctggttaagtgaagtccaa

SEQ ID NO: 698 - DNA, 120 bp, Homo sapiens
ttgtctgcctccagaggagggagttgacctagcagtggtaactggagagtgttttttgaaacctctttccaaggttagttgccaatggcatctttggaacagtgtccttcacttttgtcc

SEQ ID NO: 699 - DNA, 120 bp, Homo sapiens
tacatttctctttgttctgtgagccttttatgctggaatagttttcactgcaggctgttattgtctgcctccagaggagggagttgacctagcagtggtaactggagagtgttttttgaa

SEQ ID NO: 700 - DNA, 120 bp, Homo sapiens
agcaaattgctattttttttaaatttcatagtgtacatgagtataaggtgctgaatatgtgattgattctgagggaaaagagagataagggaaagttctcagagaaagtcaagctgaggg

SEQ ID NO: 701 - DNA, 120 bp, Homo sapiens
aaaatacagagaagtgaaaccagagatagtgggatcattctggagtctgttgcctacactgaacagtagttgagcgaaaaaggatgggcagaatgtgttggttctgggtattgcaaattc

SEQ ID NO: 702 - DNA, 120 bp, Homo sapiens
ataatttgctattcaccaagtacttcttacgtacactgtgcatgaaatgattattactttttctaatattagttttcttgattgaggcttggcaattattagtttgtatgcctttagaag

SEQ ID NO: 703 - DNA, 120 bp, Homo sapiens
atggcacttgagtgaaaaagtttaagccttctattggctctttgtgaatatcttcaacatgcatgactacaaatagaacacatggttttgttgttattgttgttgtgtttttgttttttt

SEQ ID NO: 704 - DNA, 120 bp, Homo sapiens
gatcataagcagaggtttatcccagtaggatttgcattttagaatgatgactttgggagtaaaatacagagaagtgaaaccagagatagtgggatcattctggagtctgttgcctacact

SEQ ID NO: 705 - DNA, 120 bp, Homo sapiens
tttttaccctttgaagatattgaactgttttatgaacacaatcttagaaggatttaaaaaataatttgctattcaccaagtacttcttacgtacactgtgcatgaaatgattattacttt

SEQ ID NO: 706 - DNA, 120 bp, Homo sapiens
gaacagtagttgagcgaaaaaggatgggcagaatgtgttggttctgggtattgcaaattcatggcacttgagtgaaaaagtttaagccttctattggctctttgtgaatatcttcaacat

SEQ ID NO: 707 - DNA, 120 bp, Homo sapiens
ttctaatattagttttcttgattgaggcttggcaattattagtttgtatgcctttagaaggatcataagcagaggtttatcccagtaggatttgcattttagaatgatgactttgggagt

SEQ ID NO: 708 - DNA, 120 bp, Homo sapiens
ccaactttgcgctcacatctagttctgttgcccatgtgcaagctgaatttgggcccgggcccccagatctaacatgaaactcaagtttccttctgttcaaactgtccaggcataatagtc

SEQ ID NO: 709 - DNA, 120 bp, Homo sapiens
ttaaagtccgatgcccagcagagccgtagatttttcactggccaaaaatcaacatgaaaccagatgtatctgtaaatctagtttcataacactttgtagtcaatggaaatacagtagcag

SEQ ID NO: 710 - DNA, 120 bp, Homo sapiens
ttaagaattagtaaaaatagtaaaagaacaattcattctccatccagatgttctgtccccactgtgacttatgtgctcattcagagttgtacagaaaaacctccacttaattttcacaag

SEQ ID NO: 711 - DNA, 120 bp, Homo sapiens
ccccagatctaacatgaaactcaagtttccttctgttcaaactgtccaggcataatagtcttaaagtccgatgcccagcagagccgtagatttttcactggccaaaaatcaacatgaaac

SEQ ID NO: 712 - DNA, 120 bp, Homo sapiens
cagatgtatctgtaaatctagtttcataacactttgtagtcaatggaaatacagtagcaggcagaccagaccagagtttactatttgcagtggaattaataaccacatggaaactttgcc

SEQ ID NO: 713 - DNA, 120 bp, Homo sapiens
tttggtatctgcgagatggaagataaaggtgcgaattcaaagcagttcccaccttaccctctaaattccaacataaagaggccttgaatgtccttctatcttattgtatatttcattaac

SEQ ID NO: 714 - DNA, 120 bp, Homo sapiens
gaagtgtttttaaaagatttaaaatcgaaataatataaaagaatgttaaaaacaagtaaaacatatcactagttaatcactctaccaaaattcatttttatgtttgcatatttaaccatt

SEQ ID NO: 715 - DNA, 120 bp, Homo sapiens
ctaaattccaacataaagaggccttgaatgtccttctatcttattgtatatttcattaacagaagtatgttcctagctacttagtcattctatctctattctcctttgttttaacttcag

SEQ ID NO: 716 - DNA, 120 bp, Homo sapiens
ctggagttccacatgtaacagaatcatatgggaccaaaaaattctctgtattggcttcttccctgccgtattttggctctgggaccaacaagacacccattttgcatgagctgcctgcca

SEQ ID NO: 717 - DNA, 120 bp, Homo sapiens
aggaaatttagaattgcctaaagaaaggatgtattggccaacctaataataaatcagtattagtgaatctaaagcatatttgaaaaatttgtaacatgagttgaaattcagacctgcaat

SEQ ID NO: 718 - DNA, 120 bp, Homo sapiens
acatatcactagttaatcactctaccaaaattcatttttatgtttgcatatttaaccatttttattttctatatttgtccatgaacatgtgtttttatatattgtttatattaaacatgg

SEQ ID NO: 719 - DNA, 120 bp, Homo sapiens
ttatctttagggacagaaatgttaggaagatcctagttcctcatctctttgctcctgacaaggaaatttagaattgcctaaagaaaggatgtattggccaacctaataataaatcagtat

SEQ ID NO: 720 - DNA, 120 bp, Homo sapiens
tggtgccagcttaagatgctctggctttcagctttcatggagcacgtcatgtttttaaacttatctttagggacagaaatgttaggaagatcctagttcctcatctctttgctcctgaca

SEQ ID NO: 721 - DNA, 120 bp, Homo sapiens
agaagtatgttcctagctacttagtcattctatctctattctcctttgttttaacttcagtggtgccagcttaagatgctctggctttcagctttcatggagcacgtcatgtttttaaac

SEQ ID NO: 722 - DNA, 120 bp, Homo sapiens
gcagaccagaccagagtttactatttgcagtggaattaataaccacatggaaactttgcctttggtatctgcgagatggaagataaaggtgcgaattcaaagcagttcccaccttaccct

SEQ ID NO: 723 - DNA, 120 bp, Homo sapiens
tttattttctatatttgtccatgaacatgtgtttttatatattgtttatattaaacatggttttaatcatggcttatttcttttatgttttacttcttttcctttgacataaaatattgt

SEQ ID NO: 724 - DNA, 120 bp, Homo sapiens
tagtgaatctaaagcatatttgaaaaatttgtaacatgagttgaaattcagacctgcaatgaagtgtttttaaaagatttaaaatcgaaataatataaaagaatgttaaaaacaagtaaa

SEQ ID NO: 725 - DNA, 120 bp, Homo sapiens
actgtgacttatgtgctcattcagagttgtacagaaaaacctccacttaattttcacaagctggagttccacatgtaacagaatcatatgggaccaaaaaattctctgtattggcttctt

SEQ ID NO: 726 - DNA, 120 bp, Homo sapiens
ccctgccgtattttggctctgggaccaacaagacacccattttgcatgagctgcctgccaccaactttgcgctcacatctagttctgttgcccatgtgcaagctgaatttgggcccgggc

SEQ ID NO: 727 - DNA, 120 bp, Homo sapiens
ttctctaaaaattattctaagaaaagacaaaggtgatacgaaatatatcctgagtttttatttttttcttgcatgggatttgtatatttgcacctttgcccatttatactatgatttctt

SEQ ID NO: 728 - DNA, 120 bp, Homo sapiens
aaggcccttaaacatttttaggcttaattggctgtccttgtacttagggcacatctaaaaatcctgaggcaaccactcaagagaacatgcttttgttaattcaaagggagctgtcctacg

SEQ ID NO: 729 - DNA, 120 bp, Homo sapiens
tttttttcttgcatgggatttgtatatttgcacctttgcccatttatactatgatttcttagtgtcttccctggcaattttaatgaagacttcatgtatatcaatttttccacaaatata

SEQ ID NO: 730 - DNA, 120 bp, Homo sapiens
agtgtccagaatcctctgtagtcttgggcctggtgcttgagagacccaaaggaaaggtcaatggaattacagcttagtgttagagctttcatgcatcacactaattaattaatgtcataa

SEQ ID NO: 731 - DNA, 120 bp, Homo sapiens
atggaattacagcttagtgttagagctttcatgcatcacactaattaattaatgtcataaaggtctctctcctgttatgggaaaaagcagcaaataggaacttctggtagggtgcttaaa

SEQ ID NO: 732 - DNA, 120 bp, Homo sapiens
agtgtcttccctggcaattttaatgaagacttcatgtatatcaatttttccacaaatataatctttctaaaaatatgttttttccacaatataattcagacgtattctccgaaatgttgg

SEQ ID NO: 733 - DNA, 120 bp, Homo sapiens
aggtctctctcctgttatgggaaaaagcagcaaataggaacttctggtagggtgcttaaagttggtttgatattttttattagcatttttaactaatacaagtaatacatgcttatggta

SEQ ID NO: 734 - DNA, 120 bp, Homo sapiens
gaggtagggattattttgtaactgcccccaaccttctaacctgtaatgaaacaaacactgaaggcccttaaacatttttaggcttaattggctgtccttgtacttagggcacatctaaaa

SEQ ID NO: 735 - DNA, 120 bp, Homo sapiens
gggcaagaccacttaactcttggaaaatacaggaaacgtagatttctagaggccaagaaggaggtagggattattttgtaactgcccccaaccttctaacctgtaatgaaacaaacactg

SEQ ID NO: 736 - DNA, 120 bp, Homo sapiens
atctttctaaaaatatgttttttccacaatataattcagacgtattctccgaaatgttggaaaaacttaagtaggcatcaaagcatttgaagatttgtttaaaggttgtttttataccag

SEQ ID NO: 737 - DNA, 120 bp, Homo sapiens
aaaaacttaagtaggcatcaaagcatttgaagatttgtttaaaggttgtttttataccagttttaaattgtaatttaagggtcataaaataggtgaaaattaaatcatttttcagtaagg

SEQ ID NO: 738 - DNA, 120 bp, Homo sapiens
gttggtttgatattttttattagcatttttaactaatacaagtaatacatgcttatggtagaatgataaaactgaaaaaaaaggtatgaaaatttagaagttctcctactcatgacctca

SEQ ID NO: 739 - DNA, 120 bp, Homo sapiens
ttttaaattgtaatttaagggtcataaaataggtgaaaattaaatcatttttcagtaagggggcaagaccacttaactcttggaaaatacaggaaacgtagatttctagaggccaagaag

SEQ ID NO: 740 - DNA, 120 bp, Homo sapiens
atcctgaggcaaccactcaagagaacatgcttttgttaattcaaagggagctgtcctacgagtgtccagaatcctctgtagtcttgggcctggtgcttgagagacccaaaggaaaggtca

SEQ ID NO: 741 - DNA, 120 bp, Homo sapiens
ttccttttttctagattttatttttatttaaggcatcctttgattttaacctgattttttttctctaaaaattattctaagaaaagacaaaggtgatacgaaatatatcctgagttttta

SEQ ID NO: 742 - DNA, 120 bp, Homo sapiens
tatataattgttagtataattgtatacctttattatcatacttttgcatgtatagcaataagacaaattcttagatgtttaaccattggacataaggaatgtacgcattttaagtactgg

SEQ ID NO: 743 - DNA, 120 bp, Homo sapiens
aattttttttttatttttcagattaattttcacttagagattcatcagcatatgtactatacatgtacaaatcacctgtgtgttttggatatttagttaaacaaatgtgcaaatatttta

SEQ ID NO: 744 - DNA, 120 bp, Homo sapiens
atggtttcacaaaaagagctttctactaaaaaataaaatacatacttaagcaactcagagaattttttttttatttttcagattaattttcacttagagattcatcagcatatgtactat

SEQ ID NO: 745 - DNA, 120 bp, Homo sapiens
gggcaacttttttcttgcctgtctgcctgcctaagccaacttaaataaacatcatttctgtaacttctgtgaagccttttccaatctctccactccaagacgaaggtgtttctataggca

SEQ ID NO: 746 - DNA, 120 bp, Homo sapiens
accaaaggagcatattcatttgtgttttattttcttaatggttttcgttatgaatgtgaaatgtgtatttaccttaacagaaattaagtatatttttggtctgacatatatgagaactga

SEQ ID NO: 747 - DNA, 120 bp, Homo sapiens
acctatgcagtcttccagtcttatttacacattcctttgcacatgctgtttccccgtgtggggcaacttttttcttgcctgtctgcctgcctaagccaacttaaataaacatcatttctg

SEQ ID NO: 748 - DNA, 120 bp, Homo sapiens
aaagcattggcttggctgctaactgcattctcatctttctttctctgctttggcaaagtctgggattaaatctaataccttttaaactgtttgggacttcagccagagtgacctgtcttg

SEQ ID NO: 749 - DNA, 120 bp, Homo sapiens
acatgtacaaatcacctgtgtgttttggatatttagttaaacaaatgtgcaaatattttaaccaaaggagcatattcatttgtgttttattttcttaatggttttcgttatgaatgtgaa

SEQ ID NO: 750 - DNA, 120 bp, Homo sapiens
aattcagaactgcgcagatcattccccattctaaggccctctcatgcctcctcattgcctgtaggatgagatccaagtaccttagcatagcttatgcactgtagtcacttgacctctagc

SEQ ID NO: 751 - DNA, 120 bp, Homo sapiens
atgtgtatttaccttaacagaaattaagtatatttttggtctgacatatatgagaactgaaaagcattggcttggctgctaactgcattctcatctttctttctctgctttggcaaagtc

SEQ ID NO: 752 - DNA, 120 bp, Homo sapiens
gtaggatgagatccaagtaccttagcatagcttatgcactgtagtcacttgacctctagcacctatgcagtcttccagtcttatttacacattcctttgcacatgctgtttccccgtgtg

SEQ ID NO: 753 - DNA, 120 bp, Homo sapiens
tgggattaaatctaataccttttaaactgtttgggacttcagccagagtgacctgtcttgaattcagaactgcgcagatcattccccattctaaggccctctcatgcctcctcattgcct

SEQ ID NO: 754 - DNA, 120 bp, Homo sapiens
ccctctcaaaagcaacctaaacaggatgtggggtaggctctaaagcattcctcaagccacacatggatccatcagtaaaatgtggagggcttaaggataaaaaggcttaagtacaatctc

SEQ ID NO: 755 - DNA, 120 bp, Homo sapiens
cttgtggaattgcactcactgctgggtgtgtggaaagttatgcatgtgtgctttactgtaccctctcaaaagcaacctaaacaggatgtggggtaggctctaaagcattcctcaagccac

SEQ ID NO: 756 - DNA, 120 bp, Homo sapiens
cagaaggcttaaagtcaggttagggaaaggaggcctatgaggttactgtgcagaggcagtgctgggaaataaatgaagttaaataaatttaggccatcgtggtttaaagaatggattgtg

SEQ ID NO: 757 - DNA, 120 bp, Homo sapiens
ggcaaaataataaaatcatatttgtctctagtggaatggataactatgcctaaaactgtgccctttgaaaagcaactagagagataatttctgaagtgtttgtccctacctgaatgtgtg

SEQ ID NO: 758 - DNA, 120 bp, Homo sapiens
tggccctacattttctaaatgttatgccaccctgaccaaggggcaactcctacaaagccaggcaaaataataaaatcatatttgtctctagtggaatggataactatgcctaaaactgtg

SEQ ID NO: 759 - DNA, 120 bp, Homo sapiens
ccctttgaaaagcaactagagagataatttctgaagtgtttgtccctacctgaatgtgtggcaaaattctaaactccctgaagtgtgaaagtggtttccaagccacatgcacatccagta

SEQ ID NO: 760 - DNA, 120 bp, Homo sapiens
gcaaaattctaaactccctgaagtgtgaaagtggtttccaagccacatgcacatccagtagtggtaaagggtgaaaatctaactggctaagagggcttcatagcaacattaaccaaaaag

SEQ ID NO: 761 - DNA, 120 bp, Homo sapiens
tggtttatgtagtctttgcctgcttcataattccctaggcattctatgctattctgtactcagaaggcttaaagtcaggttagggaaaggaggcctatgaggttactgtgcagaggcagt

SEQ ID NO: 762 - DNA, 120 bp, Homo sapiens
gtggtaaagggtgaaaatctaactggctaagagggcttcatagcaacattaaccaaaaagtggtttatgtagtctttgcctgcttcataattccctaggcattctatgctattctgtact

SEQ ID NO: 763 - DNA, 120 bp, Homo sapiens
acatggatccatcagtaaaatgtggagggcttaaggataaaaaggcttaagtacaatctctggccctacattttctaaatgttatgccaccctgaccaaggggcaactcctacaaagcca

SEQ ID NO: 764 - DNA, 120 bp, Homo sapiens
gctgggaaataaatgaagttaaataaatttaggccatcgtggtttaaagaatggattgtggagataagaaggataaaggaaacccagagtcaagaaaaataaaacttttcattggtgcca

SEQ ID NO: 765 - DNA, 120 bp, Homo sapiens
ttcccaggagaaagtggctgaagattccagagagaagctgaatgcagtttaattctttttgccataaacacgacaacccattttcctgcaagctgtgttagtttgctctcttcttggttc

SEQ ID NO: 766 - DNA, 120 bp, Homo sapiens
catcctcctaaggcctcagaaataaggccttattttaataagtgcaagtcagtcatttgaagactaaatcatagaatcctagaaaactagtaccgggagcaaggcaaaagaatgggatga

SEQ ID NO: 767 - DNA, 120 bp, Homo sapiens
agactaaatcatagaatcctagaaaactagtaccgggagcaaggcaaaagaatgggatgagcatgaaacatatattcagaagttgtggtgtgtaggtatataagccaagctcttttcttc

SEQ ID NO: 768 - DNA, 120 bp, Homo sapiens
ttgaaggcaaagcgcctggtgctcctgatctcatgagcacagagcatttagcctaagtctcatcctcctaaggcctcagaaataaggccttattttaataagtgcaagtcagtcatttga

SEQ ID NO: 769 - DNA, 120 bp, Homo sapiens
tttatgaagtgttgtgcatattgttgttaattttaagctgttacgttaaagaacccctaatccaactctcttgagttttatagatatcatagaagatatatcttcccttgacatagaagc

SEQ ID NO: 770 - DNA, 120 bp, Homo sapiens
gcttgtttaccacattgaatactttatctgtgtttatctaacgacagttccaccagctctttaccacttgacttttgcctaattcaaaaatataccaactatgaaacattttccttctca

SEQ ID NO: 771 - DNA, 120 bp, Homo sapiens
ttaccacttgacttttgcctaattcaaaaatataccaactatgaaacattttccttctcagtttttattctagattacattttgttcaactttatcttaatgtgtagtgtagaaagagta

SEQ ID NO: 772 - DNA, 120 bp, Homo sapiens
tcccaactcccactccagtatactaaggcagattccagagaagaaacagtggagagcaggcactgatgagggacaaagaaaagcaggctccgtctggctgcaacttgtctcttcatggca

SEQ ID NO: 773 - DNA, 120 bp, Homo sapiens
aaaagaaactaggaaagtgctatgccagagacgacatgataactttgcagaatggaaagagcttgtttaccacattgaatactttatctgtgtttatctaacgacagttccaccagctct

SEQ ID NO: 774 - DNA, 120 bp, Homo sapiens
cactgatgagggacaaagaaaagcaggctccgtctggctgcaacttgtctcttcatggcaaaaagaaactaggaaagtgctatgccagagacgacatgataactttgcagaatggaaaga

SEQ ID NO: 775 - DNA, 120 bp, Homo sapiens
gttcctattctatctagggatagagtcaggaagggcttcattgaataagtggtagcctcttgggctgagacctgagttatgagatgatgtggcaaaggagacagatggctgggggcaagg

SEQ ID NO: 776 - DNA, 120 bp, Homo sapiens
ttgttcagggtccatcttggccttcaaattaagatgccctttgagagataacattgttgttttcaaactctgttctgtgacttaagaatgagaggagaaggaagaaaagaggagaaaatt

SEQ ID NO: 777 - DNA, 120 bp, Homo sapiens
taccgtataatgcaccaagctaataggtgctttgaaagaagaccatacaagtggagatgtgttcctattctatctagggatagagtcaggaagggcttcattgaataagtggtagcctct

SEQ ID NO: 778 - DNA, 120 bp, Homo sapiens
acatggtggatgggaatcagatatgtgcatcaattatttgtgttccaatccatatagaagtaccgtataatgcaccaagctaataggtgctttgaaagaagaccatacaagtggagatgt

SEQ ID NO: 779 - DNA, 120 bp, Homo sapiens
tgagggaaaagtgcccaagcagcgtcaaggctagacactggaaatttatcaatgaaagccacatggtggatgggaatcagatatgtgcatcaattatttgtgttccaatccatatagaag

SEQ ID NO: 780 - DNA, 120 bp, Homo sapiens
tttcaaactctgttctgtgacttaagaatgagaggagaaggaagaaaagaggagaaaatttgagggaaaagtgcccaagcagcgtcaaggctagacactggaaatttatcaatgaaagcc

SEQ ID NO: 781 - DNA, 120 bp, Homo sapiens
tgggctgagacctgagttatgagatgatgtggcaaaggagacagatggctgggggcaaggtggggtcattgaaattggaggcagtagcaatataagcaaagctacaggggcatgaaaaag

SEQ ID NO: 782 - DNA, 120 bp, Homo sapiens
caaggttagattagtgaattgcaacagggtggtactgctggaaggtcacatggaaaagattgtgaaggtattgagataagaagctagaaataagctttgaatgccatcctagtactttga

SEQ ID NO: 783 - DNA, 120 bp, Homo sapiens
tggggtcattgaaattggaggcagtagcaatataagcaaagctacaggggcatgaaaaagcaaggttagattagtgaattgcaacagggtggtactgctggaaggtcacatggaaaagat

SEQ ID NO: 784 - DNA, 120 bp, Homo sapiens
tagtcactgattgactgagtggatggcagtgataggtggggtgcgttgagggaagtgtattacattaagtccaggatgactcatggttttctaagttgagtcattggggattgccatcca

SEQ ID NO: 785 - DNA, 120 bp, Homo sapiens
gtctaaccttggcttcttggttgtggattctcgtcaacatttcactgctacccaagttgtgtctgcttacatgatgctatcttccttcttttgggtttctgaagccctcagacacttggc

SEQ ID NO: 786 - DNA, 120 bp, Homo sapiens
tgagtatctcaatggggctgtttttctagttcttgtagtttctttgggccaacatgaaatgtctaaccttggcttcttggttgtggattctcgtcaacatttcactgctacccaagttgt

SEQ ID NO: 787 - DNA, 120 bp, Homo sapiens
ggtagcatgatgattgcagagctcactggactgaaagtcagatgctttacccgcctagactctagtaccaaggggaagatggagtgagatggggtaaatggggagaaattaccatttatt

SEQ ID NO: 788 - DNA, 120 bp, Homo sapiens
gtctgcttacatgatgctatcttccttcttttgggtttctgaagccctcagacacttggctgaacatttttcacatttcttaagctatatcatctgtgttttccctgccacagacaaagt

SEQ ID NO: 789 - DNA, 120 bp, Homo sapiens
gccttgggccagacctcactacacatctgtttaagagatcagggtaagctctgttcttggtgagtatctcaatggggctgtttttctagttcttgtagtttctttgggccaacatgaaat

SEQ ID NO: 790 - DNA, 120 bp, Homo sapiens
ttttttagtctctgccaatgactcctctccccatgatcaaaatcagaaaatcagtctcttatgtgttgaggagtgagacacttctcccaagtgtttaaggctaataccttgccttgtttt

SEQ ID NO: 791 - DNA, 120 bp, Homo sapiens
gggtaagggcaagtggtaaatgctgcttttctgccttaaccagtagtgtctgacagaggaggtagcatgatgattgcagagctcactggactgaaagtcagatgctttacccgcctagac

SEQ ID NO: 792 - DNA, 120 bp, Homo sapiens
tgaacatttttcacatttcttaagctatatcatctgtgttttccctgccacagacaaagtcacaaaaggactttaagataggttttggttttttttttccccagggtttttatacatttt

SEQ ID NO: 793 - DNA, 120 bp, Homo sapiens
tcagctgttattattgtgctatatgttgtgatgtgtactggagtgagatggggtaggggattttttagtctctgccaatgactcctctccccatgatcaaaatcagaaaatcagtctctt

SEQ ID NO: 794 - DNA, 120 bp, Homo sapiens
cacaaaaggactttaagataggttttggttttttttttccccagggtttttatacattttgggtaagggcaagtggtaaatgctgcttttctgccttaaccagtagtgtctgacagagga

SEQ ID NO: 795 - DNA, 120 bp, Homo sapiens
atgtgttgaggagtgagacacttctcccaagtgtttaaggctaataccttgccttgttttgccttgggccagacctcactacacatctgtttaagagatcagggtaagctctgttcttgg

SEQ ID NO: 796 - DNA, 120 bp, Homo sapiens
actgtagtgtttaatgcttttaccatctgttaaaatgattttggagtatagctagataactgatgatggttgttatatagattttttcataggttgcctgttccaaattctatgccgtgg

SEQ ID NO: 797 - DNA, 120 bp, Homo sapiens
tgatgatggttgttatatagattttttcataggttgcctgttccaaattctatgccgtggaagaagttaaatatccagaatttgacaggaaatattattctacaacagatccctggcgta

SEQ ID NO: 798 - DNA, 120 bp, Homo sapiens
tttaccacattttccagaaagagggtagctccataatgggtgagatacattttggtggctactgtagtgtttaatgcttttaccatctgttaaaatgattttggagtatagctagataac

SEQ ID NO: 799 - DNA, 120 bp, Homo sapiens
tgatggctagaaagccattggaaaaaaaaaattggctcacagaagacagcagatgtggcttgggaaatgcaaggacatgactgtaataaggatttgtctatccagccccatttatgagag

SEQ ID NO: 800 - DNA, 120 bp, Homo sapiens
tgattccaggagaaaaggacagatttgtattgtcagtgggatacgctgttaaaaaacacttttgctactaccactccagctgtcttggcatgtttgttggtgatgtaagctacagaaaat

SEQ ID NO: 801 - DNA, 120 bp, Homo sapiens
cattttcctgtttctttagcaatggattccagaaacataatgtggaaatagctctcagtccttagatttgatgacattgcagaaagaaatctggctagtcgtcccatggctgattggcta

SEQ ID NO: 802 - DNA, 120 bp, Homo sapiens
ggaaatcaccaatagggctatagcaacctgatgcatagtgacaagtaattgttctattcatggttatgtgttgtacagagcacttgctgcatgtcaggtttgagacttgagtatgcatta

SEQ ID NO: 803 - DNA, 120 bp, Homo sapiens
tgggaaatgcaaggacatgactgtaataaggatttgtctatccagccccatttatgagagtgattccaggagaaaaggacagatttgtattgtcagtgggatacgctgttaaaaaacact

SEQ ID NO: 804 - DNA, 120 bp, Homo sapiens
gtgagaaaaatattggttcagtaagatttaacaggtaaattaaaatcaagtatttgaaaacattttcctgtttctttagcaatggattccagaaacataatgtggaaatagctctcagtc

SEQ ID NO: 805 - DNA, 120 bp, Homo sapiens
tgtaaaatatttagtttggtcagtcatagggctgagaacatggtggcagttacctcctagtatctgcaagcaaaaaaagttttttcttcctatagcaattgccatctcagccacttttgc

SEQ ID NO: 806 - DNA, 120 bp, Homo sapiens
gggccatggacacccccatcttatctttaagtagatttcaaagtaaatatttgatgaatatgtaaaatatttagtttggtcagtcatagggctgagaacatggtggcagttacctcctag

SEQ ID NO: 807 - DNA, 120 bp, Homo sapiens
tttgctactaccactccagctgtcttggcatgtttgttggtgatgtaagctacagaaaatggaaatcaccaatagggctatagcaacctgatgcatagtgacaagtaattgttctattca

SEQ ID NO: 808 - DNA, 120 bp, Homo sapiens
cttagatttgatgacattgcagaaagaaatctggctagtcgtcccatggctgattggctatgatggctagaaagccattggaaaaaaaaaattggctcacagaagacagcagatgtggct

SEQ ID NO: 809 - DNA, 120 bp, Homo sapiens
tggttatgtgttgtacagagcacttgctgcatgtcaggtttgagacttgagtatgcattagggccatggacacccccatcttatctttaagtagatttcaaagtaaatatttgatgaata

SEQ ID NO: 810 - DNA, 120 bp, Homo sapiens
aagtaggagtgacctgtcttctctctttcttcacgatggggactagtgtgtgtatataaggggataatttttgtgtcacataaaatataaccttacttagaaggcaagacttccagaatg

SEQ ID NO: 811 - DNA, 120 bp, Homo sapiens
acactaaatttcaaatctcacggtggcagggaataaagatgctacctatcttaagccattacttcaccaacttctccaccaaaatattccttgtaaccacaaataagtaagcacaataga

SEQ ID NO: 812 - DNA, 120 bp, Homo sapiens
tctataaggagagaataattgtgaactctgattttatcttaaaaagtcatgtagggatgtcatgttccacaatgtgattaataaaatatattttgttactaaacacaaggaaaaatatta

SEQ ID NO: 813 - DNA, 120 bp, Homo sapiens
acttcaccaacttctccaccaaaatattccttgtaaccacaaataagtaagcacaatagatctataaggagagaataattgtgaactctgattttatcttaaaaagtcatgtagggatgt

SEQ ID NO: 814 - DNA, 120 bp, Homo sapiens
catgttccacaatgtgattaataaaatatattttgttactaaacacaaggaaaaatattatgttccataaagatgtttggtggttgcctcgacctcttttagtttgaaaagtaggtatgt

SEQ ID NO: 815 - DNA, 120 bp, Homo sapiens
atgaactaaatttcggtaatacttttaatagtaaacattgctgccctcgtgaatgaacacacactaaatttcaaatctcacggtggcagggaataaagatgctacctatcttaagccatt

SEQ ID NO: 816 - DNA, 120 bp, Homo sapiens
ccccgcaacccccacatgtatttacctttctctaaaagctctgcatagccaagaaaagtgctcttttttatttttaggatattagatatttcattttcttatggtaagacaaaagattaa

SEQ ID NO: 817 - DNA, 120 bp, Homo sapiens
tggtctgtgacttttgcatttcaccccgcttccacagtcactggtgggacttacttaagttaatcagattcttcaaagtatccccaagtcctcctttgaaaagaaagttgggggacagga

SEQ ID NO: 818 - DNA, 120 bp, Homo sapiens
ggtgatctcagctgggtgccaaggtttcctaagcccaagttccccatggttgagcctgtattgtcaggccaacagcttctagtaatccacttttatttaattaatagtgaaactgttgaa

SEQ ID NO: 819 - DNA, 120 bp, Homo sapiens
ctgtgtttgcaattaggatgttatctaggagcatattcaaaacttttgaggtttttattttagtttttctttcattatgtgctgttttagtaatatcaaagaatacatgtaatatataat

SEQ ID NO: 820 - DNA, 120 bp, Homo sapiens
ggtaagaaaaaacacaatgtcacgtgcaaattctgcacttgttctcaaagcaaacctctcctgtgtttgcaattaggatgttatctaggagcatattcaaaacttttgaggtttttattt

SEQ ID NO: 821 - DNA, 120 bp, Homo sapiens
gaattgcaagtggtgttctggttcagaaaccttccgttctatggggcactgcttttgcttcagattcataaaaccaaatgctctgcctcaagataataagtgaacgtgtaaccctcggga

SEQ ID NO: 822 - DNA, 120 bp, Homo sapiens
tagtttttctttcattatgtgctgttttagtaatatcaaagaatacatgtaatatataatttatatgtcataacaataaaattaatgttgatgagcccagattaaagaatcaacaacatt

SEQ ID NO: 823 - DNA, 120 bp, Homo sapiens
ttgtcaggccaacagcttctagtaatccacttttatttaattaatagtgaaactgttgaagaattgcaagtggtgttctggttcagaaaccttccgttctatggggcactgcttttgctt

SEQ ID NO: 824 - DNA, 120 bp, Homo sapiens
cagattcataaaaccaaatgctctgcctcaagataataagtgaacgtgtaaccctcgggaggtaagaaaaaacacaatgtcacgtgcaaattctgcacttgttctcaaagcaaacctctc

SEQ ID NO: 825 - DNA, 120 bp, Homo sapiens
gctcatgagtcagagctgcaatgtgccctttgtccacactaggtcaggatcagtgggagtgctacccaaaatattttgctagctggggagtcagggagaagcagagactgacctagtgag

SEQ ID NO: 826 - DNA, 120 bp, Homo sapiens
gccaggaggcactatctcaggtctctagtcaaaatgggttgcaattagtaaaagtccagattctgaatccccttcactatttatcttcctcttcctcctttacagttatttttgttcaag

SEQ ID NO: 827 - DNA, 120 bp, Homo sapiens
ttctgaatccccttcactatttatcttcctcttcctcctttacagttatttttgttcaaggtgcactttattaaactcatgcctaacaaacaaaactctaatgaatattttgtctttcat

SEQ ID NO: 828 - DNA, 120 bp, Homo sapiens
gctacccaaaatattttgctagctggggagtcagggagaagcagagactgacctagtgaggccaggaggcactatctcaggtctctagtcaaaatgggttgcaattagtaaaagtccaga

SEQ ID NO: 829 - DNA, 120 bp, Homo sapiens
gtgcactttattaaactcatgcctaacaaacaaaactctaatgaatattttgtctttcattgattgtaaattcaattaattagattgcttgaaaaaattttaactgtattttcactttag

SEQ ID NO: 830 - DNA, 120 bp, Homo sapiens
tttctatttcttctccatgtttcattcaggactgaggaagggggcacagtttttacccaaggaaatgacatttttagccaaaagaaatgatcttagcatttagctgaattatatattgga

SEQ ID NO: 831 - DNA, 120 bp, Homo sapiens
ttgctggctttctagttagggtagggaaaggaaggcttgtggggaatgaagataggccatgatatcaagccactgggtttgcaaatcagtagaattttttattgctttctgttgtacttg

SEQ ID NO: 832 - DNA, 120 bp, Homo sapiens
agttttctttgctcttgtcctgacatagctgttcacttggggttgaggggaggataacctttcatgttttttttttttcttcattctgatgactgtgctgaacattcaaaccaaaaggcc

SEQ ID NO: 833 - DNA, 120 bp, Homo sapiens
ggaaatgacatttttagccaaaagaaatgatcttagcatttagctgaattatatattggaagtaagctccttccatgtggaacttatggccttgctagccttggtttgttggaagtgctc

SEQ ID NO: 834 - DNA, 120 bp, Homo sapiens
agtaagctccttccatgtggaacttatggccttgctagccttggtttgttggaagtgctcttgctggctttctagttagggtagggaaaggaaggcttgtggggaatgaagataggccat

SEQ ID NO: 835 - DNA, 120 bp, Homo sapiens
gatatcaagccactgggtttgcaaatcagtagaattttttattgctttctgttgtacttgggacttgaataaaggctgatatttgtgtcttgctggtaaagtgcttgtaaagtgagtgaa

SEQ ID NO: 836 - DNA, 120 bp, Homo sapiens
agtttgtgttagtaacagaagtcaaaaaggtggagaaaggagaaaggtacttgtgaaaattttctatttcttctccatgtttcattcaggactgaggaagggggcacagtttttacccaa

SEQ ID NO: 837 - DNA, 120 bp, Homo sapiens
ggacttgaataaaggctgatatttgtgtcttgctggtaaagtgcttgtaaagtgagtgaaagttttctttgctcttgtcctgacatagctgttcacttggggttgaggggaggataacct

SEQ ID NO: 838 - DNA, 120 bp, Homo sapiens
ttcatgttttttttttttcttcattctgatgactgtgctgaacattcaaaccaaaaggccattggtggaaagtaaaggtgagtggtgagaagacaatagggtaatggaaactgtgttgga

SEQ ID NO: 839 - DNA, 120 bp, Homo sapiens
ccagaatcattgtacatcattttttcaacagaagcttcaggcatagggattatgcttggtactttatgttgtggaatggaatctggcggatgtccatgtgatctatagaaacacctaagg

SEQ ID NO: 840 - DNA, 120 bp, Homo sapiens
cttctccttcatatccacctgaaagagcttgggcgcagaagttcttgcagaaaggcagttagacaaggtgacttctgaagctccagtggccaagtattttgatggtagcctaaaagatgt

SEQ ID NO: 841 - DNA, 120 bp, Homo sapiens
agacaaggtgacttctgaagctccagtggccaagtattttgatggtagcctaaaagatgtccagaatcattgtacatcattttttcaacagaagcttcaggcatagggattatgcttggt

SEQ ID NO: 842 - DNA, 120 bp, Homo sapiens
acaaacaggctcccattagggctcattttccttcattccttagtaaggaagaagtgcttataaaatatagcagttgtgctcttgtgaatgatagcatgggcagttgtcatctccctgaag

SEQ ID NO: 843 - DNA, 120 bp, Homo sapiens
tttactataggagactgagaattaaccttccatgaaggttttaggattggctttctggcccttctccttcatatccacctgaaagagcttgggcgcagaagttcttgcagaaaggcagtt

SEQ ID NO: 844 - DNA, 120 bp, Homo sapiens
tgacaaaagctgacctttgggtagtgagaacaatgttccattttgttcaaacttgaattttttactataggagactgagaattaaccttccatgaaggttttaggattggctttctggcc

SEQ ID NO: 845 - DNA, 120 bp, Homo sapiens
cagatgtaacccagaatgtcacttgagttttgtttaatgcttaggcataagacataggaatgacaaaagctgacctttgggtagtgagaacaatgttccattttgttcaaacttgaattt

SEQ ID NO: 846 - DNA, 120 bp, Homo sapiens
actttatgttgtggaatggaatctggcggatgtccatgtgatctatagaaacacctaaggaaagtgaagaaatgagggaaaaaaaagaacaagacttttatgataatactaatcacgatc

SEQ ID NO: 847 - DNA, 120 bp, Homo sapiens
taaaatatagcagttgtgctcttgtgaatgatagcatgggcagttgtcatctccctgaagcagatgtaacccagaatgtcacttgagttttgtttaatgcttaggcataagacataggaa

SEQ ID NO: 848 - DNA, 120 bp, Homo sapiens
aacctttccttctataagagctataaaccttgagaacagtcttaaaacataggtatgtaggccacaccattcaccacgaatgtactgatactcatcagaatatggaagaagcaccagaga

SEQ ID NO: 849 - DNA, 120 bp, Homo sapiens
ttgagcaatgaataatagtcatttatgcctgcaggttaatgctgaagacctgagacttcacttgcctatttctgccattcagtgacatgtgttgcattggttttttgtgtctttccagtt

SEQ ID NO: 850 - DNA, 120 bp, Homo sapiens
gttttatttagcttagaacaactcatgtctgctcaacctctagaggcgctcagcccacattctgcagtagaaactcccattttcaggcctcttatatacggtaatgtctccttcctctaa

SEQ ID NO: 851 - DNA, 120 bp, Homo sapiens
tctcagaaatagagtcattggcaaggccctatcaaataacttaggagcctaaggaagcaaatttttgtacttgctagttccctggtttcagcagccttgtttgtacaggcaatttaggca

SEQ ID NO: 852 - DNA, 120 bp, Homo sapiens
atttttgtacttgctagttccctggtttcagcagccttgtttgtacaggcaatttaggcagtgaaggtggtcccagctggggcttggggctcagtgggtcctagaaatgaaagaaaaatt

SEQ ID NO: 853 - DNA, 120 bp, Homo sapiens
caccctacaaccatcatctttaggagaagtctcagaaaattcagcttcacactaactaacttgagcaatgaataatagtcatttatgcctgcaggttaatgctgaagacctgagacttca

SEQ ID NO: 854 - DNA, 120 bp, Homo sapiens
ggatcctgtgccatttgagaacagggaagaaaagaaatgaggttttggggagggaatcacccaactcacagaacacacagaaatccagcaaggtttcaaaacgctctacaccttagagtc

SEQ ID NO: 855 - DNA, 120 bp, Homo sapiens
agatatcttaacctcaggcttcctgccttcattgctcccgcatatagacatagactatgagattggctaatcccagagaacttccctaatcccttggcaagatccaaaaaggctcagtca

SEQ ID NO: 856 - DNA, 120 bp, Homo sapiens
gtttgaagcatctagagaaaaggtagaaagagaatgccctttaactgacctcctcagtgatagccaatcacaatgatgagtgttgattcatcattttggctaggtggcagaaatatctat

SEQ ID NO: 857 - DNA, 120 bp, Homo sapiens
gtgaaggtggtcccagctggggcttggggctcagtgggtcctagaaatgaaagaaaaattaatgatttgaaaagatttaatttcctcccttcttgttttctactctgctggctagtaaag

SEQ ID NO: 858 - DNA, 120 bp, Homo sapiens
gagggaggcaggcagaagggcaactggcagggctgcctgggaggagctctgcaatgaggtggatcctgtgccatttgagaacagggaagaaaagaaatgaggttttggggagggaatcac

SEQ ID NO: 859 - DNA, 120 bp, Homo sapiens
ccaactcacagaacacacagaaatccagcaaggtttcaaaacgctctacaccttagagtctgttaagttagggaaactctgtgagctcatagggccaaatgcacttgcctgcttgaaata

SEQ ID NO: 860 - DNA, 120 bp, Homo sapiens
tggcctccttgggacctgtctttgcattaatagttcctaggtaggtaagaactcagagtgaagaaacacatttattctcctctccagagacctgatctcaaagcctgtccattagtccct

SEQ ID NO: 861 - DNA, 120 bp, Homo sapiens
gaaaggatggctgtcagaaaaggaaatgaggatgggttccagagacttcagaccaccccaacttccccagtgagaccctggcacctccccataccctctcacctagcgggccctgtctat

SEQ ID NO: 862 - DNA, 120 bp, Homo sapiens
gcaccacagtaatagcagccatatcagatgggaaaggagttcaagtgaacaaacaagcaaattcaatagtcagatagattagattatacttgatgcttcctctgagttttacaaatatgg

SEQ ID NO: 863 - DNA, 120 bp, Homo sapiens
aagaaacacatttattctcctctccagagacctgatctcaaagcctgtccattagtccctaaccttaatctaaggtagcatcttatatctggctaaattggctcaagccctagctcctta

SEQ ID NO: 864 - DNA, 120 bp, Homo sapiens
cctttctctttcctctcactggctggaagtgttgagttccacttcagaaccagaacagagaacctttccttctataagagctataaaccttgagaacagtcttaaaacataggtatgtag

SEQ ID NO: 865 - DNA, 120 bp, Homo sapiens
aaaacagaagctgccatgttgttttcttccagtcctcagggcctacaagaaggcagctatcatttggtattactgaaaacatgccccatgttcagctcatacccccaaattacccattgc

SEQ ID NO: 866 - DNA, 120 bp, Homo sapiens
gaaaaatttgtccttattagagaggttagaagtggagaaaccccaactgagtccccagcctgttccttgggatgaatatgagactgttccttagcaaaggcttcctggcctcggccccag

SEQ ID NO: 867 - DNA, 120 bp, Homo sapiens
tagccaatcacaatgatgagtgttgattcatcattttggctaggtggcagaaatatctataaaacagaagctgccatgttgttttcttccagtcctcagggcctacaagaaggcagctat

SEQ ID NO: 868 - DNA, 120 bp, Homo sapiens
agagcagagaatgaaacagagcactcatctagaggtagtgtgtcagcaagcccaggcactgcaccacagtaatagcagccatatcagatgggaaaggagttcaagtgaacaaacaagcaa

SEQ ID NO: 869 - DNA, 120 bp, Homo sapiens
gattggctaatcccagagaacttccctaatcccttggcaagatccaaaaaggctcagtcacaccctacaaccatcatctttaggagaagtctcagaaaattcagcttcacactaactaac

SEQ ID NO: 870 - DNA, 120 bp, Homo sapiens
gccacaccattcaccacgaatgtactgatactcatcagaatatggaagaagcaccagagagtttgaagcatctagagaaaaggtagaaagagaatgccctttaactgacctcctcagtga

SEQ ID NO: 871 - DNA, 120 bp, Homo sapiens
catttggtattactgaaaacatgccccatgttcagctcatacccccaaattacccattgctactgtttatgctgggctaatatgaagcccagggccctaatgtctaggtctaggcagtaa

SEQ ID NO: 872 - DNA, 120 bp, Homo sapiens
ccacccagggcttaagcttcctgcttatccacttcaccctgtattgagggctttcttctcaaagagacattgatgaggagcccctagagagagatgctgtgctctgggaccagacccctt

SEQ ID NO: 873 - DNA, 120 bp, Homo sapiens
tactgtttatgctgggctaatatgaagcccagggccctaatgtctaggtctaggcagtaaggcctagagcagtgcctaaagagcctgagagcagtgccttcctttcttcagagtactcat

SEQ ID NO: 874 - DNA, 120 bp, Homo sapiens
gcaaggtcttcttcaaaagagccgctgaaggtaaagggtcttgcacatgcacttctctttccctttctcctttaccttccagagagagacactaacctttcagggcccaggattttatca

SEQ ID NO: 875 - DNA, 120 bp, Homo sapiens
atcactttgatatttggtctggttttgttataggtctccctttggatgaggtaaagttacaaacctgggttcatatcatttaattagtctgaaaatgttgcctggacaccaccttcagtt

SEQ ID NO: 876 - DNA, 120 bp, Homo sapiens
tctgcagtagaaactcccattttcaggcctcttatatacggtaatgtctccttcctctaaccacccagggcttaagcttcctgcttatccacttcaccctgtattgagggctttcttctc

SEQ ID NO: 877 - DNA, 120 bp, Homo sapiens
ggcctagagcagtgcctaaagagcctgagagcagtgccttcctttcttcagagtactcatgaaaggatggctgtcagaaaaggaaatgaggatgggttccagagacttcagaccacccca

SEQ ID NO: 878 - DNA, 120 bp, Homo sapiens
gtcactaaattgttattttcagaaaacaggggaaatgctcaatcacattgtgaaagggaagattttgctgtcatatcatacatcccacatgggagctttctgcagaagttagagctgaag

SEQ ID NO: 879 - DNA, 120 bp, Homo sapiens
tgttaagttagggaaactctgtgagctcatagggccaaatgcacttgcctgcttgaaatatgaaaaatcagcaatggattccttgaaaaacaatgaaaagggaaccttctgagccccttg

SEQ ID NO: 880 - DNA, 120 bp, Homo sapiens
tgaaaaatcagcaatggattccttgaaaaacaatgaaaagggaaccttctgagccccttggttattttgacatatggaccatagatttcagtcctgagccctttgaaggtaggagaaggt

SEQ ID NO: 881 - DNA, 120 bp, Homo sapiens
tggagactgccagggaccatgttttgcccattgactattactttccaccccagaagacctgcctgatctgtggagatgaagcttctgggtgtcactatggagctctcacatgtggaagct

SEQ ID NO: 882 - DNA, 120 bp, Homo sapiens
attcaatagtcagatagattagattatacttgatgcttcctctgagttttacaaatatgggtcactaaattgttattttcagaaaacaggggaaatgctcaatcacattgtgaaagggaa

SEQ ID NO: 883 - DNA, 120 bp, Homo sapiens
gcctgatctgtggagatgaagcttctgggtgtcactatggagctctcacatgtggaagctgcaaggtcttcttcaaaagagccgctgaaggtaaagggtcttgcacatgcacttctcttt

SEQ ID NO: 884 - DNA, 120 bp, Homo sapiens
tgttccttgggatgaatatgagactgttccttagcaaaggcttcctggcctcggccccagaaagggagtgttctcactcttcagcagactatcagtctctgcacctgctccctcctgttg

SEQ ID NO: 885 - DNA, 120 bp, Homo sapiens
gattttgctgtcatatcatacatcccacatgggagctttctgcagaagttagagctgaaggagggaggcaggcagaagggcaactggcagggctgcctgggaggagctctgcaatgaggt

SEQ ID NO: 886 - DNA, 120 bp, Homo sapiens
gttaaacaccagtattcacctctgccccaactttccccaaagaggtacttcctgccaaggcctttctctttcctctcactggctggaagtgttgagttccacttcagaaccagaacagag

SEQ ID NO: 887 - DNA, 120 bp, Homo sapiens
acttccccagtgagaccctggcacctccccataccctctcacctagcgggccctgtctatagagcagagaatgaaacagagcactcatctagaggtagtgtgtcagcaagcccaggcact

SEQ ID NO: 888 - DNA, 120 bp, Homo sapiens
aaccttaatctaaggtagcatcttatatctggctaaattggctcaagccctagctccttagttttatttagcttagaacaactcatgtctgctcaacctctagaggcgctcagcccacat

SEQ ID NO: 889 - DNA, 120 bp, Homo sapiens
aaagggagtgttctcactcttcagcagactatcagtctctgcacctgctccctcctgttgtggcctccttgggacctgtctttgcattaatagttcctaggtaggtaagaactcagagtg

SEQ ID NO: 890 - DNA, 120 bp, Homo sapiens
ccctttctcctttaccttccagagagagacactaacctttcagggcccaggattttatcatctcagaaatagagtcattggcaaggccctatcaaataacttaggagcctaaggaagcaa

SEQ ID NO: 891 - DNA, 120 bp, Homo sapiens
aatgatttgaaaagatttaatttcctcccttcttgttttctactctgctggctagtaaaggaaaaatttgtccttattagagaggttagaagtggagaaaccccaactgagtccccagcc

SEQ ID NO: 892 - DNA, 120 bp, Homo sapiens
aaagagacattgatgaggagcccctagagagagatgctgtgctctgggaccagaccccttgttaaacaccagtattcacctctgccccaactttccccaaagaggtacttcctgccaagg

SEQ ID NO: 893 - DNA, 120 bp, Homo sapiens
cttgcctatttctgccattcagtgacatgtgttgcattggttttttgtgtctttccagtttggagactgccagggaccatgttttgcccattgactattactttccaccccagaagacct

SEQ ID NO: 894 - DNA, 120 bp, Homo sapiens
aaacctgggttcatatcatttaattagtctgaaaatgttgcctggacaccaccttcagttagatatcttaacctcaggcttcctgccttcattgctcccgcatatagacatagactatga

SEQ ID NO: 895 - DNA, 120 bp, Homo sapiens
gcttgttgaaacatcaaattatacctgtcttagagaaaatagaaacaaaaatctttctcttccttacttgcttgttgtagtcagttaactcggactgagtattcagagtcttgattatca

SEQ ID NO: 896 - DNA, 120 bp, Homo sapiens
tctttctcctcccatctgtgaagctgttggattgattttactgccatcattatccctgtttgaaggcagggggctgtcttattacccaaagaggacatttattgatttggttttcttttt

SEQ ID NO: 897 - DNA, 120 bp, Homo sapiens
gtgagggtgaggctgggcagggagggtctgcatagaaaaaagggtgcggtgagaaaaaataatgctactaagccatgagggtaaaatgaccaaattctggttgagagaaacttggtcaaa

SEQ ID NO: 898 - DNA, 120 bp, Homo sapiens
ggaagagtgggaagagggagtcctacattttctccttgtcagtaatgttggagaattggggtgagggtgaggctgggcagggagggtctgcatagaaaaaagggtgcggtgagaaaaaat

SEQ ID NO: 899 - DNA, 120 bp, Homo sapiens
tccttacttgcttgttgtagtcagttaactcggactgagtattcagagtcttgattatcacttaattcatagtttcataaatctctggaatgggcataggtacaggacttaaaagcctgg

SEQ ID NO: 900 - skipped

SEQ ID NO: 901 - DNA, 120 bp, Homo sapiens
ctgggttagaaacaggcatggagggaaatagttggtttatggagtgggtaggatgagtggggtggtgaaagggaaggcattttggatgctaagagaccaggaagtcaaagcaaggcaata

SEQ ID NO: 902 - DNA, 120 bp, Homo sapiens
tgtcattggtgcagggctcagcacagagtcagttgtaatctggacaggttttgttgttgaggaagagtgggaagagggagtcctacattttctccttgtcagtaatgttggagaattggg

SEQ ID NO: 903 - DNA, 120 bp, Homo sapiens
ccatttttacaatgcatctttatcgcccatatggcctttctggaggtggttttcagtctggcttgttgaaacatcaaattatacctgtcttagagaaaatagaaacaaaaatctttctct

SEQ ID NO: 904 - DNA, 120 bp, Homo sapiens
catctcagacagaaatatgtttttagctttggtggtttataacagatgggacttttaggctgtcattggtgcagggctcagcacagagtcagttgtaatctggacaggttttgttgttga

SEQ ID NO: 905 - DNA, 120 bp, Homo sapiens
aatgctactaagccatgagggtaaaatgaccaaattctggttgagagaaacttggtcaaagtgtgtatggggagagaaagttggtcaaagtctgtgtctgagtgcttggtgggatgaact

SEQ ID NO: 906 - DNA, 120 bp, Homo sapiens
cttaattcatagtttcataaatctctggaatgggcataggtacaggacttaaaagcctggcatctcagacagaaatatgtttttagctttggtggtttataacagatgggacttttaggc

SEQ ID NO: 907 - DNA, 120 bp, Homo sapiens
tgaaggcagggggctgtcttattacccaaagaggacatttattgatttggttttctttttccatttttacaatgcatctttatcgcccatatggcctttctggaggtggttttcagtctg

SEQ ID NO: 908 - DNA, 120 bp, Homo sapiens
gtgtgtatggggagagaaagttggtcaaagtctgtgtctgagtgcttggtgggatgaactctgggttagaaacaggcatggagggaaatagttggtttatggagtgggtaggatgagtgg

SEQ ID NO: 909 - DNA, 120 bp, Homo sapiens
tttcagcaaactcacatgtatttatacctgcataagtttttggtcttgctttcctagaaggtgactaatcccagatcctaatcaattaaagaagcaatcttcagatggggatagagccag

SEQ ID NO: 910 - DNA, 120 bp, Homo sapiens
gtgcattatgtgctcaagaatttactatttttcagacattttctagtaaaacattgaagattatatgtccatttgttttgtacacatggagtgctgtttggtacacatcataaaattgaa

SEQ ID NO: 911 - DNA, 120 bp, Homo sapiens
agcagagagtttttggtgcatatcagtgtccccttcactccctgacttttcaagtaacatttcccagaggcaaattaactctgctaagaggatctgcttgcagcttcaacagagccttca

SEQ ID NO: 912 - DNA, 120 bp, Homo sapiens
actgtagtttacattctgaactcaaagaattacaccatcctcactgatgtttacaataggtcccaatttagtttctttagcaaattttatgtaagtatggctttgattctctctctcact

SEQ ID NO: 913 - DNA, 120 bp, Homo sapiens
ttatatgtccatttgttttgtacacatggagtgctgtttggtacacatcataaaattgaaactgtagtttacattctgaactcaaagaattacaccatcctcactgatgtttacaatagg

SEQ ID NO: 914 - DNA, 120 bp, Homo sapiens
tcaggtatctttggccaaggagttgactgatcctgactttgcgagtcctagagatcttttcacaaagctcctctcatgtttctgcctctgattttcttaaatgtcacagacagactttag

SEQ ID NO: 915 - DNA, 120 bp, Homo sapiens
ccaggtttttgttagggaagaaatgcaagtgaaccctcattgaactctttctgtcctttaaatccattctttcccacctcaactcatgtggaattgaatgttgcctctagtttggagtct

SEQ ID NO: 916 - DNA, 120 bp, Homo sapiens
ttcccagaggcaaattaactctgctaagaggatctgcttgcagcttcaacagagccttcatcaggtatctttggccaaggagttgactgatcctgactttgcgagtcctagagatctttt

SEQ ID NO: 917 - DNA, 120 bp, Homo sapiens
aatccattctttcccacctcaactcatgtggaattgaatgttgcctctagtttggagtctagcagagagtttttggtgcatatcagtgtccccttcactccctgacttttcaagtaacat

SEQ ID NO: 918 - DNA, 120 bp, Homo sapiens
tcccaatttagtttctttagcaaattttatgtaagtatggctttgattctctctctcactccaggtttttgttagggaagaaatgcaagtgaaccctcattgaactctttctgtccttta

SEQ ID NO: 919 - DNA, 120 bp, Homo sapiens
atagcacttgcaccattatgtaaatatctgtaatgcttacataacttttgtcacttgcaagaccttttgagtccattgccttctgctaccatgccttaccaatttcctagtcccttatta

SEQ ID NO: 920 - DNA, 120 bp, Homo sapiens
ggcttgcagaagaaataatttagctctgtaactcattgaagttggtgcccacccaagtctctgtcagtgcccaattcgggagccatgccaagaatttgccattgctgcttcatggtggcc

SEQ ID NO: 921 - DNA, 120 bp, Homo sapiens
ccagaggagaaactgaaaatatttcaaacattttctagacttctgtgttgtaaatttgtggataactatgaactatatatgaatgaacttttctggatgacacatatattccagatggta

SEQ ID NO: 922 - DNA, 120 bp, Homo sapiens
tctgtcttcgcacactcaccggcttaattctgggcctccccataacacgactagaccacaggcttgcagaagaaataatttagctctgtaactcattgaagttggtgcccacccaagtct

SEQ ID NO: 923 - DNA, 120 bp, Homo sapiens
gtggcttcttggtcctgagcttatttactaaacaagagaaaaaataaataagtctagaaatgctagaagaggatacttttttgttttaatgatctagtagatcactcctccttgcaatac

SEQ ID NO: 924 - DNA, 120 bp, Homo sapiens
tagaaagtagatcccaggaggccagcagagttgtggatctgccatatattacctcatgattctgtcttcgcacactcaccggcttaattctgggcctccccataacacgactagaccaca

SEQ ID NO: 925 - DNA, 120 bp, Homo sapiens
tgctagaagaggatacttttttgttttaatgatctagtagatcactcctccttgcaatacccagaggagaaactgaaaatatttcaaacattttctagacttctgtgttgtaaatttgtg

SEQ ID NO: 926 - DNA, 120 bp, Homo sapiens
tggtatcttagtaggccagtcaaagtttgaacaacttgttagcacagaatacctggcctagtggcttcttggtcctgagcttatttactaaacaagagaaaaaataaataagtctagaaa

SEQ ID NO: 927 - DNA, 120 bp, Homo sapiens
gctttagctaatcacttctgtacttataaatctgcataggttttatgtttttccatctcttggtatcttagtaggccagtcaaagtttgaacaacttgttagcacagaatacctggccta

SEQ ID NO: 928 - DNA, 120 bp, Homo sapiens
aaaaggaagggctttggggactctctggtaccaagtgtcatggaaaaactgtgtgtctcatagaaagtagatcccaggaggccagcagagttgtggatctgccatatattacctcatgat

SEQ ID NO: 929 - DNA, 120 bp, Homo sapiens
ctgtcagtgcccaattcgggagccatgccaagaatttgccattgctgcttcatggtggccttgtgcctgcttatttatagcctgtgcattttatgaaacagggattaataagaagttgcc

SEQ ID NO: 930 - DNA, 120 bp, Homo sapiens
ttgtgcctgcttatttatagcctgtgcattttatgaaacagggattaataagaagttgccatagcacttgcaccattatgtaaatatctgtaatgcttacataacttttgtcacttgcaa

SEQ ID NO: 931 - DNA, 120 bp, Homo sapiens
gataactatgaactatatatgaatgaacttttctggatgacacatatattccagatggtaaaaaggaagggctttggggactctctggtaccaagtgtcatggaaaaactgtgtgtctca

SEQ ID NO: 932 - DNA, 120 bp, Homo sapiens
ttcagggaattgctgggtgactatcaaactctggtagttcatttttgcagttggctgctgttgtgaggataagagttagactcactttctcttcagagatagaaattatgtattaattct

SEQ ID NO: 933 - DNA, 120 bp, Homo sapiens
ttgtgaggataagagttagactcactttctcttcagagatagaaattatgtattaattctctgggttctagacccacagcaaggagcatactgctcctcaaaataactgaattctgcgag

SEQ ID NO: 934 - DNA, 120 bp, Homo sapiens
aagccatcattgtaaaacaacaatatcttcagttatagtagccatgtgtgcaacttctggaaactgttattcagattttcatgttccttccctgtctcttcatagctaggcagctgcttt

SEQ ID NO: 935 - DNA, 120 bp, Homo sapiens
acccttatcaattttttatgtgcctctccatattctgcagtcagaagcttcttcagtcctttcagggaattgctgggtgactatcaaactctggtagttcatttttgcagttggctgctg

SEQ ID NO: 936 - DNA, 120 bp, Homo sapiens
aaactgttattcagattttcatgttccttccctgtctcttcatagctaggcagctgctttcagccttgtacagatgctagtgagctttctacctacaaacctgcagaaaattgaactgag

SEQ ID NO: 937 - DNA, 120 bp, Homo sapiens
tcccaggctgtgttgtgactactgaggcactccagtgaaatcactattcctcctatctagactaatgcctgtctctgcagagcacctcataagaacaggcctggtagtaatatcctcatg

SEQ ID NO: 938 - DNA, 120 bp, Homo sapiens
atttggaggtgaaagactcttgataaagggaacaaggtttagaattctcagtccctttgctcccaggctgtgttgtgactactgaggcactccagtgaaatcactattcctcctatctag

SEQ ID NO: 939 - DNA, 120 bp, Homo sapiens
ctgggttctagacccacagcaaggagcatactgctcctcaaaataactgaattctgcgagaagccatcattgtaaaacaacaatatcttcagttatagtagccatgtgtgcaacttctgg

SEQ ID NO: 940 - DNA, 120 bp, Homo sapiens
cagccttgtacagatgctagtgagctttctacctacaaacctgcagaaaattgaactgagatttggaggtgaaagactcttgataaagggaacaaggtttagaattctcagtccctttgc

SEQ ID NO: 941 - DNA, 120 bp, Homo sapiens
aacaattccttaagttacataagcacattcctacaggtcacaagctcatttacttacaggatggttgatttggtcacaggttatttcatgaaaatacttaaaagatttgcagtgttcaaa

SEQ ID NO: 942 - DNA, 120 bp, Homo sapiens
aaatgtagattaatggttccttggctctttggtttgagccttctcagcagagcatcccacggagtgttttccatggggccacgagcaagagaaatccacttccctcctcctcaatgtcag

SEQ ID NO: 943 - DNA, 120 bp, Homo sapiens
tatcgctaacatcacagggaacttgtcttcctaagaaaattccaagcacttaaaaccgctggtagttcatcagcaactctcttcattagatgtgcgagggacatgtgggccatagtcctt

SEQ ID NO: 944 - DNA, 120 bp, Homo sapiens
ctactaacttatattcttcaggggaaagttctgattctgatgagacccagcatggtagctcttaattcactgttgtcacacgactatagaacaggaagcacaacttaacacctgtgctca

SEQ ID NO: 945 - DNA, 120 bp, Homo sapiens
cttaattcactgttgtcacacgactatagaacaggaagcacaacttaacacctgtgctcatgagaattttgctccttatgaccaagctaaagaaagagcttagacaggatgtgtggctat

SEQ ID NO: 946 - DNA, 120 bp, Homo sapiens
tgagaattttgctccttatgaccaagctaaagaaagagcttagacaggatgtgtggctataaatgtagattaatggttccttggctctttggtttgagccttctcagcagagcatcccac

SEQ ID NO: 947 - DNA, 120 bp, Homo sapiens
aaaatagagaatattgtctttcaggatagaattaaaaagtcatagaggcagcaacttgttttcctatattagggttttaaaattctgtttttccttcctctcctgggtcagatcattgtg

SEQ ID NO: 948 - DNA, 120 bp, Homo sapiens
tggatggaccttgatttcattgtggtatctgtatgtggaccctgaagaccatggacttctaacaattccttaagttacataagcacattcctacaggtcacaagctcatttacttacagg

SEQ ID NO: 949 - DNA, 120 bp, Homo sapiens
ttcctatattagggttttaaaattctgtttttccttcctctcctgggtcagatcattgtgtggatggaccttgatttcattgtggtatctgtatgtggaccctgaagaccatggacttct

SEQ ID NO: 950 - DNA, 120 bp, Homo sapiens
aagtactatgcctaggcaacaagaaaggcagcaatgaagagcaacagcagagtcaaatatgagagaaggaagttaagaaagatgttaagtactgtggggagtaactgagaaaccaccaag

SEQ ID NO: 951 - DNA, 120 bp, Homo sapiens
gagagaaggaagttaagaaagatgttaagtactgtggggagtaactgagaaaccaccaagtatcgctaacatcacagggaacttgtcttcctaagaaaattccaagcacttaaaaccgct

SEQ ID NO: 952 - DNA, 120 bp, Homo sapiens
ggtagttcatcagcaactctcttcattagatgtgcgagggacatgtgggccatagtccttctactaacttatattcttcaggggaaagttctgattctgatgagacccagcatggtagct

SEQ ID NO: 953 - DNA, 120 bp, Homo sapiens
ggagtgttttccatggggccacgagcaagagaaatccacttccctcctcctcaatgtcagaaaatagagaatattgtctttcaggatagaattaaaaagtcatagaggcagcaacttgtt

SEQ ID NO: 954 - DNA, 120 bp, Homo sapiens
agaattatgtgaggcagcatagtaagcatttatggcccttggttcctagaaggagcttagtccctgatagtcatctctgcctttgccattgtgtgagactgtcttctgtaactgtatgtc

SEQ ID NO: 955 - DNA, 120 bp, Homo sapiens
tccctgatagtcatctctgcctttgccattgtgtgagactgtcttctgtaactgtatgtcttcctccctagtaagttaatgagtaataaaggtattctatagtgagaggactctgtaaga

SEQ ID NO: 956 - DNA, 120 bp, Homo sapiens
gactctcctgcctaatagctaattagcagagtcacagaggtcattaccttgcaattctcaagaattatgtgaggcagcatagtaagcatttatggcccttggttcctagaaggagcttag

SEQ ID NO: 957 - DNA, 120 bp, Homo sapiens
cagaataccagtcttgtctttggtaaggattttatagacccatcctgactacagtgatatccaacatggctatgtaatgactggcactttccccacataacatatatttattccacactc

SEQ ID NO: 958 - DNA, 120 bp, Homo sapiens
gcaagctgtttgacaggccttcagttggctctttgtaccttgctccctccgcatgctgagctgtccatagctgccctaggctggtgtctgggattttcggaagaaggttactatccaggt

SEQ ID NO: 959 - DNA, 120 bp, Homo sapiens
cctctttactttttacctcccagtacactgtgagtaacattccccagccagcccagccagcacgtgttcattgcctctcttgacttccagactttggacttgaaggtgtcagagctctct

SEQ ID NO: 960 - DNA, 120 bp, Homo sapiens
gatatggaggtatagagtgatttcccacctacctagtgagcactactgagatattcaagtactctctacccaagaattctattgatataaaggtaaaaaacttgatcttaggtctaatat

SEQ ID NO: 961 - DNA, 120 bp, Homo sapiens
gtttaagttcaaattattgttccatgggagcagagatagatataggaaacaaaaaaaagggatatggaggtatagagtgatttcccacctacctagtgagcactactgagatattcaagt

SEQ ID NO: 962 - DNA, 120 bp, Homo sapiens
agtagacatcctgtccttgaggttccttaactctgctcagcttcagaatacagaaggggttggttcttcatttgtgttgtttataactaaaagcctcctactccccacttttttgcatag

SEQ ID NO: 963 - DNA, 120 bp, Homo sapiens
actgtccaggagaaaagctagcaaggtcataaattattctccatattttccagccattggtttcccttgtccagccagaggtgtgtctcaaagtatgctgaggccagattcaatagaaac

SEQ ID NO: 964 - DNA, 120 bp, Homo sapiens
atgtcacttggaaagagttttctttgtctcttttgcaacttgacaatgactagccagcaagtttaagttcaaattattgttccatgggagcagagatagatataggaaacaaaaaaaagg

SEQ ID NO: 965 - DNA, 120 bp, Homo sapiens
gtgtatctttgtccccaacaagataagtctgacctccccagcaaattcaagtcctaagccactgtccaggagaaaagctagcaaggtcataaattattctccatattttccagccattgg

SEQ ID NO: 966 - DNA, 120 bp, Homo sapiens
aagctccatctgaagagaggggaataacacccagccaagagccctcagggcccatcagtaagtagacatcctgtccttgaggttccttaactctgctcagcttcagaatacagaaggggt

SEQ ID NO: 967 - DNA, 120 bp, Homo sapiens
tactggtactcagtaagttttgtatccttttcctagagtgagtcttggtcataggcatgcgtatacttgcagcgtccctgggtaggccgaaagagcaaataagagatggtatctatggta

SEQ ID NO: 968 - DNA, 120 bp, Homo sapiens
acatccctctgtcttcacccactcttcagttctgattcttttaaaagcagccaaccaaaaccagcaagtacatactgcttatctctgacttccaccagaatcaacttcagatcttgtcca

SEQ ID NO: 969 - DNA, 120 bp, Homo sapiens
tggttcttcatttgtgttgtttataactaaaagcctcctactccccacttttttgcatagcttcttctgccatcccacctgtgtagcctcttcaactcccccaaaactcctctgtagccc

SEQ ID NO: 970 - DNA, 120 bp, Homo sapiens
cttcttctgccatcccacctgtgtagcctcttcaactcccccaaaactcctctgtagcccatgtcacttggaaagagttttctttgtctcttttgcaacttgacaatgactagccagcaa

SEQ ID NO: 971 - DNA, 120 bp, Homo sapiens
ttccccaggtaaaggaggccttgggttggcataagatttcacttctctttagagttacttaattagggaccagaaaggccatcagcatttgtatgagaatataacaaaggtcaatctctt

SEQ ID NO: 972 - DNA, 120 bp, Homo sapiens
ctgagccagcacctgtgtaaataatttttaaagctccttttcctgaagctggatgaatatttttaaaaactaagctggattgtcttttatctagcatgccgtctcctacattcctagtgc

SEQ ID NO: 973 - DNA, 120 bp, Homo sapiens
gagggagacatttctttcagaagcaaggtaatactttggtctggtctatgactctattttgtttaaaatgaaactatggcagtatagtggtattcattctgcttcccataggttaacttt

SEQ ID NO: 974 - DNA, 120 bp, Homo sapiens
ccagcaagtacatactgcttatctctgacttccaccagaatcaacttcagatcttgtccaaagctccatctgaagagaggggaataacacccagccaagagccctcagggcccatcagta

SEQ ID NO: 975 - DNA, 120 bp, Homo sapiens
tttcccttgtccagccagaggtgtgtctcaaagtatgctgaggccagattcaatagaaacctgagccagcacctgtgtaaataatttttaaagctccttttcctgaagctggatgaatat

SEQ ID NO: 976 - DNA, 120 bp, Homo sapiens
gtttaaaatgaaactatggcagtatagtggtattcattctgcttcccataggttaactttacatccctctgtcttcacccactcttcagttctgattcttttaaaagcagccaaccaaaa

SEQ ID NO: 977 - DNA, 120 bp, Homo sapiens
aattagggaccagaaaggccatcagcatttgtatgagaatataacaaaggtcaatctcttcctctttactttttacctcccagtacactgtgagtaacattccccagccagcccagccag

SEQ ID NO: 978 - DNA, 120 bp, Homo sapiens
tatggacctcttggaggaatgtggtttggttatagtggtattgtcttgtctgttgtgggggagggagacatttctttcagaagcaaggtaatactttggtctggtctatgactctatttt

SEQ ID NO: 979 - DNA, 120 bp, Homo sapiens
cacgtgttcattgcctctcttgacttccagactttggacttgaaggtgtcagagctctctgtgtatctttgtccccaacaagataagtctgacctccccagcaaattcaagtcctaagcc

SEQ ID NO: 980 - DNA, 120 bp, Homo sapiens
gtatacttgcagcgtccctgggtaggccgaaagagcaaataagagatggtatctatggtattccccaggtaaaggaggccttgggttggcataagatttcacttctctttagagttactt

SEQ ID NO: 981 - DNA, 120 bp, Homo sapiens
ttttaaaaactaagctggattgtcttttatctagcatgccgtctcctacattcctagtgctatggacctcttggaggaatgtggtttggttatagtggtattgtcttgtctgttgtgggg

SEQ ID NO: 982 - DNA, 120 bp, Homo sapiens
taatttgtctgttgctatttatacggataatttgatagtagttatttttggacatggatagctttgaagccttacagatgagtccatccccaagtacccaaaactaaagaaagttggcta

SEQ ID NO: 983 - DNA, 120 bp, Homo sapiens
gctttgaagccttacagatgagtccatccccaagtacccaaaactaaagaaagttggctagagtgatgacaaggtggcagcacagagctccctgcgttctgggccctgtcccctagctag

SEQ ID NO: 984 - DNA, 120 bp, Homo sapiens
attacagcacattttcaatgctccaattatgtcactgtagaaatgctaatgtggattaaataatttgtctgttgctatttatacggataatttgatagtagttatttttggacatggata

SEQ ID NO: 985 - DNA, 120 bp, Homo sapiens
gagtgatgacaaggtggcagcacagagctccctgcgttctgggccctgtcccctagctagagagaactccaggctataagcatttgtattctcatagtccaatggcagggaagaagggct

SEQ ID NO: 986 - DNA, 120 bp, Homo sapiens
gaatcaaacactgataacagtacttcctagtacacaaatgagaaatcagtccctcatcaaattacagcacattttcaatgctccaattatgtcactgtagaaatgctaatgtggattaaa

SEQ ID NO: 987 - DNA, 120 bp, Homo sapiens
ctggtgggcctgccttccaaggtcagttgcagctttagcactataaagagcacctacctgcggcagatacaatgtgatgggacatgacagagaaaaaatctataagcagagcctccccat

SEQ ID NO: 988 - DNA, 120 bp, Homo sapiens
tcccaggcattgaaacaatcctaaccaagactggcatagtacaatgagcctgtccctatcagcaggtttggaagccttaacaacaacaacaaaaacaataataatggtgatgataatcat

SEQ ID NO: 989 - DNA, 120 bp, Homo sapiens
cggcagatacaatgtgatgggacatgacagagaaaaaatctataagcagagcctccccattcccaggcattgaaacaatcctaaccaagactggcatagtacaatgagcctgtccctatc

SEQ ID NO: 990 - DNA, 120 bp, Homo sapiens
tcaatttggttgataatttggttgaaatgaatttatttcattttttattccatccttacaatggaagattagtgcttgtttcccacccaaggataccaggatatttcaggggctgtatta

SEQ ID NO: 991 - DNA, 120 bp, Homo sapiens
ttataaaacaactaattgatttaaatcaaagcccaaacagaagtgtttgctaattttatttcaatttggttgataatttggttgaaatgaatttatttcattttttattccatccttaca

SEQ ID NO: 992 - DNA, 120 bp, Homo sapiens
gagatcactgtagcaattgattggtttaaatcaaagcccccaaaaaaatgttattgagaattataaaacaactaattgatttaaatcaaagcccaaacagaagtgtttgctaattttatt

SEQ ID NO: 993 - DNA, 120 bp, Homo sapiens
tttttaaatttccttagcatccatttccaccattggaaattcagggtcaaaacaggggtttgggattggagcatgtctatcacagataaccaatcatgtgttatgacttaagaatttatg

SEQ ID NO: 994 - DNA, 120 bp, Homo sapiens
ccctcatgcatgcatataatttatagagtcactgttttgctcggttgtcctcatgcctctatattattggaggtttagattgtttccatatactcaggttgtattcatgtcctttttttc

SEQ ID NO: 995 - DNA, 120 bp, Homo sapiens
atattattggaggtttagattgtttccatatactcaggttgtattcatgtcctttttttctttttaaatttccttagcatccatttccaccattggaaattcagggtcaaaacaggggtt

SEQ ID NO: 996 - DNA, 120 bp, Homo sapiens
aagcccaccaccctaacacaacaaatacttaaaacttgtcttcatttcctttaggtctggccctcatgcatgcatataatttatagagtcactgttttgctcggttgtcctcatgcctct

SEQ ID NO: 997 - DNA, 120 bp, Homo sapiens
aaagggccctctacctgaagatatcttgctactgatgctgtctcacagtgtctgaaactcccatcatatgtggaattgttttggaaggctttgcctcctgggacacattcagccataatc

SEQ ID NO: 998 - DNA, 120 bp, Homo sapiens
aagaaatagtattgagcattagactgtcagtatgtccattagcaagactgtggaggaatggaatcaccaatattatattttataggggatacagaatacaagagaagttctgaagagaaa

SEQ ID NO: 999 - DNA, 120 bp, Homo sapiens
tgggattggagcatgtctatcacagataaccaatcatgtgttatgacttaagaatttatgaaagggccctctacctgaagatatcttgctactgatgctgtctcacagtgtctgaaactc

SEQ ID NO: 1000 - DNA, 120 bp, Homo sapiens
attcttatgtagaataggaaggcttagatacagcatgaaagctgcaggctttgaggagccagaggtcaaatgaaagcattgagtatttgtttagatgaaagaacagaaagggaaaaagaa

SEQ ID NO: 1001 - DNA, 120 bp, Homo sapiens
agaggtcaaatgaaagcattgagtatttgtttagatgaaagaacagaaagggaaaaagaagcagaggaagggatagtagagagaaatgtataagttttatccatttaacttgtaattgtg

SEQ ID NO: 1002 - DNA, 120 bp, Homo sapiens
gaatcaccaatattatattttataggggatacagaatacaagagaagttctgaagagaaaattcttatgtagaataggaaggcttagatacagcatgaaagctgcaggctttgaggagcc

SEQ ID NO: 1003 - DNA, 120 bp, Homo sapiens
cacattgaaaacctctactggagtgcattgtgtctggtgggcttcaaccttaattcttaagtatgtgaaaacacatcacctatctggaggtttacactttctgctaatgactttattttt

SEQ ID NO: 1004 - DNA, 120 bp, Homo sapiens
gtatgtgaaaacacatcacctatctggaggtttacactttctgctaatgactttatttttaagcccaccaccctaacacaacaaatacttaaaacttgtcttcatttcctttaggtctgg

SEQ ID NO: 1005 - DNA, 120 bp, Homo sapiens
ccatcatatgtggaattgttttggaaggctttgcctcctgggacacattcagccataatcaagaaatagtattgagcattagactgtcagtatgtccattagcaagactgtggaggaatg

SEQ ID NO: 1006 - DNA, 120 bp, Homo sapiens
gactccaacatcattacagaactataaattacatgtggaaaagaaaggcctcctatgttagaatagaaaataaaatgctgtggggttgagggacagaggtgctgtctaggaagtcagata

SEQ ID NO: 1007 - DNA, 120 bp, Homo sapiens
tcactgagatggtttggggattgtggcttccagatgatcagattttcttttttaggtagagactccaacatcattacagaactataaattacatgtggaaaagaaaggcctcctatgtta

SEQ ID NO: 1008 - DNA, 120 bp, Homo sapiens
agtaggtgtatatggggaattggagggagataggtggctgtgtttagtaattggttgacttcactgagatggtttggggattgtggcttccagatgatcagattttcttttttaggtaga

SEQ ID NO: 1009 - DNA, 120 bp, Homo sapiens
catagaactgtaggggtcaaggccaaaggggacgtcctgttccaagtcaccttctttggacattagaaaaccacgaggggtttggaaatcagaaaaccagcagaggcaggaaaactcagg

SEQ ID NO: 1010 - DNA, 120 bp, Homo sapiens
actgctgatgtggagtagaggcagctttgtctgctgtgtgataaccaaacctttacgaatagtaggtgtatatggggaattggagggagataggtggctgtgtttagtaattggttgact

SEQ ID NO: 1011 - DNA, 120 bp, Homo sapiens
cattagaaaaccacgaggggtttggaaatcagaaaaccagcagaggcaggaaaactcagggcagcatgggagattcagtatatacaaaaaggttcacaccagtaatcaaacagaatttta

SEQ ID NO: 1012 - DNA, 120 bp, Homo sapiens
tgagatcacttgaaactagggagagatgtgtgagttctgggcaaccagtagttggctttacatagaactgtaggggtcaaggccaaaggggacgtcctgttccaagtcaccttctttgga

SEQ ID NO: 1013 - DNA, 120 bp, Homo sapiens
gaatagaaaataaaatgctgtggggttgagggacagaggtgctgtctaggaagtcagatagcgttttccagttctgtccctcagagttccttgtcctcattgagactcaatttctcttac

SEQ ID NO: 1014 - DNA, 120 bp, Homo sapiens
gcagcatgggagattcagtatatacaaaaaggttcacaccagtaatcaaacagaattttaactgctgatgtggagtagaggcagctttgtctgctgtgtgataaccaaacctttacgaat

SEQ ID NO: 1015 - DNA, 120 bp, Homo sapiens
agtatgttgcctcaaggaggccctcactgttctaggaaatataattccagagtttgctgactcacaccatggaatatatgcataaaatggatcctgcagataagcctttctctgactagt

SEQ ID NO: 1016 - DNA, 120 bp, Homo sapiens
taataacacaaagatcactctcgactagcaagcccttttatgatggtgtgagcatttgacacccttgttgctagtaacatcagtgagtgacctgacccatttttggaacagaatatgatc

SEQ ID NO: 1017 - DNA, 120 bp, Homo sapiens
aaaaaaacaatgaagagctttctttgggcattagacactttcccataaggtggctgactctcttttagtcatgtcagcttggcccaatcttcacttggtagcccttctttcttcttcatt

SEQ ID NO: 1018 - DNA, 120 bp, Homo sapiens
gaacaaactagaaatccagtatagaaaataaaaataggattatattccttggaatctcagaaaaaaacaatgaagagctttctttgggcattagacactttcccataaggtggctgactc

SEQ ID NO: 1019 - DNA, 120 bp, Homo sapiens
cccactgttctttctcatctcaaccctgagtataagtacagatcacattccttgggttcttagaaaataatagaaatgaactctcattcatcaaaatgcccattagtaaatactgaggga

SEQ ID NO: 1020 - DNA, 120 bp, Homo sapiens
tcttttagtcatgtcagcttggcccaatcttcacttggtagcccttctttcttcttcattaatccatctcctatgctcctatggggtcctagagaaatgcccatcatgtacacacacatc

SEQ ID NO: 1021 - DNA, 120 bp, Homo sapiens
acccttgttgctagtaacatcagtgagtgacctgacccatttttggaacagaatatgatcagtatgttgcctcaaggaggccctcactgttctaggaaatataattccagagtttgctga

SEQ ID NO: 1022 - DNA, 120 bp, Homo sapiens
aatccatctcctatgctcctatggggtcctagagaaatgcccatcatgtacacacacatctaataacacaaagatcactctcgactagcaagcccttttatgatggtgtgagcatttgac

SEQ ID NO: 1023 - DNA, 120 bp, Homo sapiens
tagaaaataatagaaatgaactctcattcatcaaaatgcccattagtaaatactgagggagaacaaactagaaatccagtatagaaaataaaaataggattatattccttggaatctcag

SEQ ID NO: 1024 - DNA, 120 bp, Homo sapiens
ctggcctcccttgacccattccattcattatctaagggactccaagccagcattccacagagtgccctcaccaaactcactaagactgaaggcgaaccaggattccaaacagccattatg

SEQ ID NO: 1025 - DNA, 120 bp, Homo sapiens
tccatacagatactactattctttaggaaaacgttaaaatcacatgatcttccaggacctgggctgcttctttaagaagcatgttacagaaagctttattggccaacaacatattgaaag

SEQ ID NO: 1026 - DNA, 120 bp, Homo sapiens
caacttgaggaagagaggtgatattatcggaatgaatttctttgttgtaagttataaatgtatgggcttttccaatcccatcacccttaaaactttatttgttttctgcagtgagggtgt

SEQ ID NO: 1027 - DNA, 120 bp, Homo sapiens
cagcagacaggatttggatctggcatttggtaacagggcagtttccaaagttgctgtacgcaacttgaggaagagaggtgatattatcggaatgaatttctttgttgtaagttataaatg

SEQ ID NO: 1028 - DNA, 120 bp, Homo sapiens
cccaacaagtgatcagtagtcagaaaatggccaagaaataccatggggtgtgccttcccataacagcttatctttgtgttttagttgcaaggttactaaaagcctgtgcagggtttatgg

SEQ ID NO: 1029 - DNA, 120 bp, Homo sapiens
aaaggaaagagagagagacttagggtttgcaaaataagataccctgttgattctttttattccatacagatactactattctttaggaaaacgttaaaatcacatgatcttccaggacct

SEQ ID NO: 1030 - DNA, 120 bp, Homo sapiens
taacagcttatctttgtgttttagttgcaaggttactaaaagcctgtgcagggtttatggcaaaagtaaaacttgctccaggagcaagcccttgtttcattgtctaatgttcttaatccc

SEQ ID NO: 1031 - DNA, 120 bp, Homo sapiens
atgtggactctctgaaattgttataaggtctttttctttgtttttttcttgatgcccaagctgccaagggtagtactggcagtggtgggcagacaaggaggtgatagcaaactttgtcct

SEQ ID NO: 1032 - DNA, 120 bp, Homo sapiens
caaaagtaaaacttgctccaggagcaagcccttgtttcattgtctaatgttcttaatccccagcagacaggatttggatctggcatttggtaacagggcagtttccaaagttgctgtacg

SEQ ID NO: 1033 - DNA, 120 bp, Homo sapiens
tatgggcttttccaatcccatcacccttaaaactttatttgttttctgcagtgagggtgtctccgttgtctttaatatgcttgctttgagttcatggatgaacattcctgcctggctgac

SEQ ID NO: 1034 - DNA, 120 bp, Homo sapiens
agtgccctcaccaaactcactaagactgaaggcgaaccaggattccaaacagccattatgaaaggaaagagagagagacttagggtttgcaaaataagataccctgttgattctttttat

SEQ ID NO: 1035 - DNA, 120 bp, Homo sapiens
ctccgttgtctttaatatgcttgctttgagttcatggatgaacattcctgcctggctgacatgtggactctctgaaattgttataaggtctttttctttgtttttttcttgatgcccaag

SEQ ID NO: 1036 - DNA, 120 bp, Homo sapiens
ctgccaagggtagtactggcagtggtgggcagacaaggaggtgatagcaaactttgtcctctggcctcccttgacccattccattcattatctaagggactccaagccagcattccacag

SEQ ID NO: 1037 - DNA, 120 bp, Homo sapiens
ctgaatatctttacatggtaataacacaatggaaagcttgcaaaatagacagaggctaggggaagaaggattgagtgtgaatatagcctcttataaatcgagaggaatggtctgtgtctt

SEQ ID NO: 1038 - DNA, 120 bp, Homo sapiens
agtttcctatgccaaccccaggcagattacatttaattttatctgatttatatagagagtttctatgtaatgttttattcttaaaaatagtttactataaaaaactcaactggtttgatt

SEQ ID NO: 1039 - DNA, 120 bp, Homo sapiens
ccacaacgctaagcctgcatggaagaggagaaaaagagtggcctgacaagagaagttcccagtttcctatgccaaccccaggcagattacatttaattttatctgatttatatagagagt

SEQ ID NO: 1040 - DNA, 120 bp, Homo sapiens
tctaaaatgttccacaaagccctggggtggggggctcctacagagtttcgctaaggcaaaccacaacgctaagcctgcatggaagaggagaaaaagagtggcctgacaagagaagttccc

SEQ ID NO: 1041 - DNA, 120 bp, Homo sapiens
ggccttaagtattaaagtattaagtattaaagtgatatgtaaccaagtatattgtttggtaacttcatttttgttattattttaacaaaccaatatattgtgaatatacttccaagtgaa

SEQ ID NO: 1042 - DNA, 120 bp, Homo sapiens
gtgatgccactaatagtgggtaacttctctgccttacctcctctgttccaaacaggatttttcagaatgaacaaattaaaagaatcataatcagacactaaccccaagccatactgcatg

SEQ ID NO: 1043 - DNA, 120 bp, Homo sapiens
gttcatataggtcctgcattaacaccatatatataaagtgtgagaaatactacattcttcaggattctctgtaggttaacaatgaagatgatgactcaaccctttctttgtttgcataat

SEQ ID NO: 1044 - DNA, 120 bp, Homo sapiens
actatgaagaaagacacagagcaacatcagacagtcaagaatttcagagccagctggcatgcagtggacctcatgccagcccattttatgactatttaggtagtcaagggtttaagattt

SEQ ID NO: 1045 - DNA, 120 bp, Homo sapiens
ttctaataagacagttattatgcatttcaatgagtgatttctttgcagctctagagtgtggccttacctacttcaacatgagaagatttttgtattttgtcagtcatttcacaatgactt

SEQ ID NO: 1046 - DNA, 120 bp, Homo sapiens
ttcagaatgaacaaattaaaagaatcataatcagacactaaccccaagccatactgcatggcagcaccaatgggactgacagaaaacaacagaaataggaagaaatcctacagagaaaca

SEQ ID NO: 1047 - DNA, 120 bp, Homo sapiens
caggattacatcccatttacacagttctctgtcacttgaatacagagaagggatccacaaggccatatgcttcctagacaaagagaaaagatttctgccacactcagaacgctttgtctt

SEQ ID NO: 1048 - DNA, 120 bp, Homo sapiens
aacttgaaagctgtctcatggcctttgaatcatacttaagttttatgatggaaggatacgactatgaagaaagacacagagcaacatcagacagtcaagaatttcagagccagctggcat

SEQ ID NO: 1049 - DNA, 120 bp, Homo sapiens
gcagtggacctcatgccagcccattttatgactatttaggtagtcaagggtttaagatttttctaataagacagttattatgcatttcaatgagtgatttctttgcagctctagagtgtg

SEQ ID NO: 1050 - DNA, 120 bp, Homo sapiens
aggattctctgtaggttaacaatgaagatgatgactcaaccctttctttgtttgcataatgtgatgccactaatagtgggtaacttctctgccttacctcctctgttccaaacaggattt

SEQ ID NO: 1051 - DNA, 120 bp, Homo sapiens
tccctgtgttaatttttcagtctcttaggttatagaggaccttctagaaccaccttacagcaggattacatcccatttacacagttctctgtcacttgaatacagagaagggatccacaa

SEQ ID NO: 1052 - DNA, 120 bp, Homo sapiens
gccttacctacttcaacatgagaagatttttgtattttgtcagtcatttcacaatgacttttagtgagcccttcattatagactgtggatacaactttgctgttggaaattaacagtgtc

SEQ ID NO: 1053 - DNA, 120 bp, Homo sapiens
aaacaactgggtataatgtttgtaatatctgaggagggggagctgcctaggaagttgtattccctgtgttaatttttcagtctcttaggttatagaggaccttctagaaccaccttacag

SEQ ID NO: 1054 - DNA, 120 bp, Homo sapiens
ggccatatgcttcctagacaaagagaaaagatttctgccacactcagaacgctttgtcttcagactataatcacccacaccatatttcctttggatccactttccagatttttgtgctgg

SEQ ID NO: 1055 - DNA, 120 bp, Homo sapiens
ttagtgagcccttcattatagactgtggatacaactttgctgttggaaattaacagtgtcaaacaactgggtataatgtttgtaatatctgaggagggggagctgcctaggaagttgtat

SEQ ID NO: 1056 - DNA, 120 bp, Homo sapiens
gcagcaccaatgggactgacagaaaacaacagaaataggaagaaatcctacagagaaacaaacttgaaagctgtctcatggcctttgaatcatacttaagttttatgatggaaggatacg

SEQ ID NO: 1057 - DNA, 120 bp, Homo sapiens
ttaggacttacagaaaaaagattcctttcatatccatctttgcaatcctcaaccacttctgtcactattatgtgtcatttcaaacattaaattcctcattctgctttgaaggaacacatg

SEQ ID NO: 1058 - DNA, 120 bp, Homo sapiens
gagagtgattattgttactgttaagaactctgatagcctcatccatatatttggagaaattgaataaataataggaaagaaataatagcatcccaatgattttaccttggctctaccatc

SEQ ID NO: 1059 - DNA, 120 bp, Homo sapiens
tgaataaataataggaaagaaataatagcatcccaatgattttaccttggctctaccatcatttggggaagtgataattcagataggagaagtgacttggaagcagtcttgagagattgc

SEQ ID NO: 1060 - DNA, 120 bp, Homo sapiens
tgtcatgtgtacccatttgtatgttttggtgtgttttatgctttatgtgatcacccacatatgcacagataattccaaaatccagtgtgtgggtgttgtattccctgtgttaattattca

SEQ ID NO: 1061 - DNA, 120 bp, Homo sapiens
ccttgagttttgggagagctttagagaacaaagacaagagactaaatgattctagatgtaagagacaatgttgcaataagttactatcctaaaaagacagaatacagggacaagagacta

SEQ ID NO: 1062 - DNA, 120 bp, Homo sapiens
ttattttggatagtttcttgcttaccagtaatacttaagtcctttacattaaaaaaaaaaaactctgtaaatatattgcagaagaaatccagacatccttcaagattcttagagctggaa

SEQ ID NO: 1063 - DNA, 120 bp, Homo sapiens
atttggggaagtgataattcagataggagaagtgacttggaagcagtcttgagagattgcctgttccatcccctatctttgtccttaaaccaaattgtacagataaataaggtcttattt

SEQ ID NO: 1064 - DNA, 120 bp, Homo sapiens
gtcacactcaaacacctatgcactcacacatacatgaatacacacatgtacattagcatgtttatgcttatgttgcatgtgactggcaacatcagtgcctttctaaggcaatgttaacta

SEQ ID NO: 1065 - DNA, 120 bp, Homo sapiens
ctgttccatcccctatctttgtccttaaaccaaattgtacagataaataaggtcttatttttaggacttacagaaaaaagattcctttcatatccatctttgcaatcctcaaccacttct

SEQ ID NO: 1066 - DNA, 120 bp, Homo sapiens
atgcacagataattccaaaatccagtgtgtgggtgttgtattccctgtgttaattattcagtcacactcaaacacctatgcactcacacatacatgaatacacacatgtacattagcatg

SEQ ID NO: 1067 - DNA, 120 bp, Homo sapiens
tttatgcttatgttgcatgtgactggcaacatcagtgcctttctaaggcaatgttaactaccttgagttttgggagagctttagagaacaaagacaagagactaaatgattctagatgta

SEQ ID NO: 1068 - DNA, 120 bp, Homo sapiens
gtcactattatgtgtcatttcaaacattaaattcctcattctgctttgaaggaacacatgtgtcatgtgtacccatttgtatgttttggtgtgttttatgctttatgtgatcacccacat

SEQ ID NO: 1069 - DNA, 120 bp, Homo sapiens
agagacaatgttgcaataagttactatcctaaaaagacagaatacagggacaagagactattattttggatagtttcttgcttaccagtaatacttaagtcctttacattaaaaaaaaaa

SEQ ID NO: 1070 - DNA, 120 bp, Homo sapiens
ttgacaaactagggtccaagaaagtattttcctggggaagatgagatttctcaaagaaggcacgcactttctaacctaagcttatttcagtaatcaatgtaacaagctggtcttgatgat

SEQ ID NO: 1071 - DNA, 120 bp, Homo sapiens
gaacgcactagaattggatacaggacctgtttttgaggagctaacacccaaaggctgaacagcactcgtagcaccgtcctttctgtgcacatatggtagtcctcagtttgcaacagaaat

SEQ ID NO: 1072 - DNA, 120 bp, Homo sapiens
cacgcactttctaacctaagcttatttcagtaatcaatgtaacaagctggtcttgatgattgcagcagtaccaatactgtgggagtgtaccagttctagaacagctacaacattggaatt

SEQ ID NO: 1073 - DNA, 120 bp, Homo sapiens
acttactcctgagtaaattgtaaagaatatctacgttttaggttgccttgttttagaccaagaggtacccagagaaaaggtgtgaactatgctaaggaaattatccgagttccaaattga

SEQ ID NO: 1074 - DNA, 120 bp, Homo sapiens
agcactcgtagcaccgtcctttctgtgcacatatggtagtcctcagtttgcaacagaaataaagctgttagcaaattatgtgttctatttatgcaaataaaatcttgtggtatgctagaa

SEQ ID NO: 1075 - DNA, 120 bp, Homo sapiens
aaaaaaaaaaaaatcatgcttttccgctataacctctctcattcacagagtgattctctttcagaagggcaatctagaactattatgggagccatattccattggtggtgcaaccatttc

SEQ ID NO: 1076 - DNA, 120 bp, Homo sapiens
tgcagcagtaccaatactgtgggagtgtaccagttctagaacagctacaacattggaattgaacgcactagaattggatacaggacctgtttttgaggagctaacacccaaaggctgaac

SEQ ID NO: 1077 - DNA, 120 bp, Homo sapiens
tcagaagggcaatctagaactattatgggagccatattccattggtggtgcaaccatttcttgacaaactagggtccaagaaagtattttcctggggaagatgagatttctcaaagaagg

SEQ ID NO: 1078 - DNA, 120 bp, Homo sapiens
agaggtacccagagaaaaggtgtgaactatgctaaggaaattatccgagttccaaattgaaaaaaaaaaaaaatcatgcttttccgctataacctctctcattcacagagtgattctctt

SEQ ID NO: 1079 - DNA, 120 bp, Homo sapiens
ggggcctccgattcagtgtgcttcttcaggtaagtcacttccctggaactcctccttggaatgagagttgtactgttgtgatttttaacagttccttcaagccaagcattttggaatcct

SEQ ID NO: 1080 - DNA, 120 bp, Homo sapiens
tgatgtatagttaaatttatgtttttagttgtttttttttctactctcaaatatcaatcactctttagtttctctttctttttccgaccacaagcattcttcctctgcttaaagaagctt

SEQ ID NO: 1081 - DNA, 120 bp, Homo sapiens
ccctaaaatcccagtctatccagtaagccaaagcacagcaataaatttgaggaaaaaataccagggacttagagacagaaaggagtgaggggatgcagaagctgaagctggagcacggtt

SEQ ID NO: 1082 - DNA, 120 bp, Homo sapiens
gaaagaagaaaggaaaattaaaggaaaaagaacaaataaaacgttaaaaaggaggaaaggaaaaaggatcctttactacaataaaactaatcttatgttcttgcaagtagcactttaagt

SEQ ID NO: 1083 - DNA, 120 bp, Homo sapiens
aaaagaagttctttgctgacctggttactactgaacctactacataaaatagcctactataatagatgcatttatgtgcctaatcttcactttttaggcttagtaaagggagaggaaagc

SEQ ID NO: 1084 - DNA, 120 bp, Homo sapiens
ctctttagtttctctttctttttccgaccacaagcattcttcctctgcttaaagaagcttccctaaaatcccagtctatccagtaagccaaagcacagcaataaatttgaggaaaaaata

SEQ ID NO: 1085 - DNA, 120 bp, Homo sapiens
gcaagcatgagaagttctgcgtgtttcagagcagccaaggatgtatttttgcctattcctgctggtgactctgtgtgtctatgcatccatctgctatatttacatgtttagtcagtcaat

SEQ ID NO: 1086 - DNA, 120 bp, Homo sapiens
aaaaaggatcctttactacaataaaactaatcttatgttcttgcaagtagcactttaagtaaaagaagttctttgctgacctggttactactgaacctactacataaaatagcctactat

SEQ ID NO: 1087 - DNA, 120 bp, Homo sapiens
aatagatgcatttatgtgcctaatcttcactttttaggcttagtaaagggagaggaaagctgatgtatagttaaatttatgtttttagttgtttttttttctactctcaaatatcaatca

SEQ ID NO: 1088 - DNA, 120 bp, Homo sapiens
ccagggacttagagacagaaaggagtgaggggatgcagaagctgaagctggagcacggttgcaagcatgagaagttctgcgtgtttcagagcagccaaggatgtatttttgcctattcct

SEQ ID NO: 1089 - DNA, 120 bp, Homo sapiens
tgacaactccaggtgttcatgacagtgatctttgttactctgttggcttcatcgaacttccttttacttgctgtgattcactacatagagtgggctttatctctgatttttataacctgc

SEQ ID NO: 1090 - DNA, 120 bp, Homo sapiens
cttttacttgctgtgattcactacatagagtgggctttatctctgatttttataacctgcaagactgggggtatgatcaccagcaatctaaaaacagttagaaatcccatggagttatct

SEQ ID NO: 1091 - DNA, 120 bp, Homo sapiens
tttgtagaaattttcctctactaatattatgaaaaataagcatcttattagctcgagtgtaattctatgcatgattacaggtatcaataggaagaaacattgactgagttcaaatctctt

SEQ ID NO: 1092 - DNA, 120 bp, Homo sapiens
gacttttggtaaaagtagagcaccttattttaaaaaccattgagtagtcctaatagtggagatatcatcaggatctgaattgttcatccctaaaaaaaacaccaatggaaatcaaacaat

SEQ ID NO: 1093 - DNA, 120 bp, Homo sapiens
aattctatgcatgattacaggtatcaataggaagaaacattgactgagttcaaatctcttctacgccatgctaaaggggtgacaagttccacaatggatcattttctcatgggcatttct

SEQ ID NO: 1094 - DNA, 120 bp, Homo sapiens
atacccgaagaaagagactctggaaactcattatcaggtctatcaactcttgtatttgttctcccagggaaacagaagtacctgtgcgccagcagaaatgattgcactattgataaattc

SEQ ID NO: 1095 - DNA, 120 bp, Homo sapiens
ctacgccatgctaaaggggtgacaagttccacaatggatcattttctcatgggcatttctgacttttggtaaaagtagagcaccttattttaaaaaccattgagtagtcctaatagtgga

SEQ ID NO: 1096 - DNA, 120 bp, Homo sapiens
atactctgtccacttttttcatgtggtaggatataatttcatatcttttctgttctagaaatacccgaagaaagagactctggaaactcattatcaggtctatcaactcttgtatttgtt

SEQ ID NO: 1097 - DNA, 120 bp, Homo sapiens
aagactgggggtatgatcaccagcaatctaaaaacagttagaaatcccatggagttatcttttgtagaaattttcctctactaatattatgaaaaataagcatcttattagctcgagtgt

SEQ ID NO: 1098 - DNA, 120 bp, Homo sapiens
atagtgccaaattaaactgtttgaatatttaggttctgtatgatcaaattgtttggtgccatactctgtccacttttttcatgtggtaggatataatttcatatcttttctgttctagaa

SEQ ID NO: 1099 - DNA, 120 bp, Homo sapiens
ctcccagggaaacagaagtacctgtgcgccagcagaaatgattgcactattgataaattccgaaggaaaaattgtccatcttgtcgtcttcggaaatgttatgaagcagggatgactctg

SEQ ID NO: 1100 - DNA, 120 bp, Homo sapiens
tgtaattctgaggatctctaggtctgagcatgtgtatgtgtgtgcgcttctatgtatctgtgacaactccaggtgttcatgacagtgatctttgttactctgttggcttcatcgaacttc

SEQ ID NO: 1101 - DNA, 120 bp, Homo sapiens
gatatcatcaggatctgaattgttcatccctaaaaaaaacaccaatggaaatcaaacaatatagtgccaaattaaactgtttgaatatttaggttctgtatgatcaaattgtttggtgcc

SEQ ID NO: 1102 - DNA, 120 bp, Homo sapiens
gcctgtgtataacagatagtttcactatactatataaccgtcagatgcaggcttgtaaattaatttgttggtgacaatgtttcagtacattttcaaattgattcattggtatagtactca

SEQ ID NO: 1103 - DNA, 120 bp, Homo sapiens
cgttcctctatcataaggctctctgtcccacaaacctgtctaccatgagtgttgtcaccattccagaaaggcttgacatcagttgattgagacttatattttccctctccaaactccccc

SEQ ID NO: 1104 - DNA, 120 bp, Homo sapiens
aagcattttttttcagtagcacagtaacgtgatagatggaagatacagctctttcaagggcgttcctctatcataaggctctctgtcccacaaacctgtctaccatgagtgttgtcacca

SEQ ID NO: 1105 - DNA, 120 bp, Homo sapiens
tctctttggagcctgtgactaggctgccacacagagccaatttcctatcctatctctcccaaagatgagcaggtgttttaataatttccttttctttgcaaagctattgaccatttccaa

SEQ ID NO: 1106 - DNA, 120 bp, Homo sapiens
aagcatccacatcaaatgagactcagatatctgagaaaactcaaccttgttttggtttgcttggtgcaccccaaagaaatccaacaattgaggtctacagtggagaagaagtaggactgg

SEQ ID NO: 1107 - DNA, 120 bp, Homo sapiens
tctaatttagaccagttgtctatctctggctttctgtgaggtgttcaataccttgtctgcctatgtgcacatttatagacaacaactagttctcttatcctggagcagggccatgtgtgg

SEQ ID NO: 1108 - DNA, 120 bp, Homo sapiens
tgttgttcagtggtagctaggatgagagaactaagaaatccagaacagtcagaggtgcaggatgacccaggcataggcgcaggatgacccaggcacaggctgatcctgaacacctgggaa

SEQ ID NO: 1109 - DNA, 120 bp, Homo sapiens
ttctccatatgaatgttgcagccccatgttgagggttcttatacactcaactgtcaattatttagccttctgtgaattatgtatagtataaaagatagggactctcaagtagggaacctc

SEQ ID NO: 1110 - DNA, 120 bp, Homo sapiens
ttcttctttgatgcttccaaggggaccagccatgctctagacacaggctgaccctttcataggcaacgtggccatcagccagctggtgccttttttttaatccttatctataccaatccc

SEQ ID NO: 1111 - DNA, 120 bp, Homo sapiens
atctcttcatgtttacatctgcccaatgccagggtcctcgctgctgcctgctacttccaaaaagatgtgtctttcatgagaaaaacaagatcattaatccacttcgatttggaaatggaa

SEQ ID NO: 1112 - DNA, 120 bp, Homo sapiens
aaagatgtgtctttcatgagaaaaacaagatcattaatccacttcgatttggaaatggaatttgaagaaaggcaagcctatttctgagtgcctgcaactgtagcctcatacccaattatt

SEQ ID NO: 1113 - DNA, 120 bp, Homo sapiens
tatcccttagctaactgctgcctatgttgtagggccagccacctcgaatgagaagctacttctctttggagcctgtgactaggctgccacacagagccaatttcctatcctatctctccc

SEQ ID NO: 1114 - DNA, 120 bp, Homo sapiens
tttgaagaaaggcaagcctatttctgagtgcctgcaactgtagcctcatacccaattattcattattagcctggaaaacccaagtgcctagaatccaaccctctcccctctcctcttaag

SEQ ID NO: 1115 - DNA, 120 bp, Homo sapiens
ggtcagggagtacagaggcaaaggcaggaagggtgacaaagtgattgacaagaaaaaatgttctccatatgaatgttgcagccccatgttgagggttcttatacactcaactgtcaatta

SEQ ID NO: 1116 - DNA, 120 bp, Homo sapiens
atcttcatatagataactatatcctccccatcctcacagggcagtagtattatttaaacagaacaaagtacctcacatgaattgacccaggctggatgagagacaatttcaaaagaatca

SEQ ID NO: 1117 - DNA, 120 bp, Homo sapiens
taatttgttggtgacaatgtttcagtacattttcaaattgattcattggtatagtactcaaatttgagtgggcttggtgaacacaatgaagacaagctgagaagtgctgtgactggcctt

SEQ ID NO: 1118 - DNA, 120 bp, Homo sapiens
gatgacccaggcataggcgcaggatgacccaggcacaggctgatcctgaacacctgggaatatcccttagctaactgctgcctatgttgtagggccagccacctcgaatgagaagctact

SEQ ID NO: 1119 - DNA, 120 bp, Homo sapiens
ttggcttgccatctggcaatatgaattgcaagtccactttgatgcaggtaaagtttaatggtaacaaaagtcctcataacatttggatgcaaatcttaacattaattccatgtctcagcc

SEQ ID NO: 1120 - DNA, 120 bp, Homo sapiens
gtaacaaaagtcctcataacatttggatgcaaatcttaacattaattccatgtctcagccaacattctccattattaagcagcctgtgatgtgattacagtgaaccacttttgaaaagga

SEQ ID NO: 1121 - DNA, 120 bp, Homo sapiens
gaacaaagtacctcacatgaattgacccaggctggatgagagacaatttcaaaagaatcatctcaagtagcgtccagtactcccaaacatcacaggtagatgttctgtgagtggctttcc

SEQ ID NO: 1122 - DNA, 120 bp, Homo sapiens
gtgagtgaggatgcatctgactgggcagggcccccaggggacttaatgatactggcctgatgttgttcagtggtagctaggatgagagaactaagaaatccagaacagtcagaggtgcag

SEQ ID NO: 1123 - DNA, 120 bp, Homo sapiens
cattccggggctcagcattagagcaggcggtgtgaagcagggatcaggagccaacagaaggtgagtgaggatgcatctgactgggcagggcccccaggggacttaatgatactggcctga

SEQ ID NO: 1124 - DNA, 120 bp, Homo sapiens
ttggtgcaccccaaagaaatccaacaattgaggtctacagtggagaagaagtaggactggggtcagggagtacagaggcaaaggcaggaagggtgacaaagtgattgacaagaaaaaatg

SEQ ID NO: 1125 - DNA, 120 bp, Homo sapiens
tttagccttctgtgaattatgtatagtataaaagatagggactctcaagtagggaacctcttggcttgccatctggcaatatgaattgcaagtccactttgatgcaggtaaagtttaatg

SEQ ID NO: 1126 - DNA, 120 bp, Homo sapiens
aaagatgagcaggtgttttaataatttccttttctttgcaaagctattgaccatttccaaaagcattttttttcagtagcacagtaacgtgatagatggaagatacagctctttcaaggg

SEQ ID NO: 1127 - DNA, 120 bp, Homo sapiens
aggcaacgtggccatcagccagctggtgccttttttttaatccttatctataccaatccccattccggggctcagcattagagcaggcggtgtgaagcagggatcaggagccaacagaag

SEQ ID NO: 1128 - DNA, 120 bp, Homo sapiens
ttccagaaaggcttgacatcagttgattgagacttatattttccctctccaaactcccccatctcttcatgtttacatctgcccaatgccagggtcctcgctgctgcctgctacttccaa

SEQ ID NO: 1129 - DNA, 120 bp, Homo sapiens
aacattctccattattaagcagcctgtgatgtgattacagtgaaccacttttgaaaaggagcctgtgtataacagatagtttcactatactatataaccgtcagatgcaggcttgtaaat

SEQ ID NO: 1130 - DNA, 120 bp, Homo sapiens
ctatgtgcacatttatagacaacaactagttctcttatcctggagcagggccatgtgtggatcttcatatagataactatatcctccccatcctcacagggcagtagtattatttaaaca

SEQ ID NO: 1131 - DNA, 120 bp, Homo sapiens
cattattagcctggaaaacccaagtgcctagaatccaaccctctcccctctcctcttaagtctaatttagaccagttgtctatctctggctttctgtgaggtgttcaataccttgtctgc

SEQ ID NO: 1132 - DNA, 120 bp, Homo sapiens
tctcaagtagcgtccagtactcccaaacatcacaggtagatgttctgtgagtggctttccaagcatccacatcaaatgagactcagatatctgagaaaactcaaccttgttttggtttgc

SEQ ID NO: 1133 - DNA, 120 bp, Homo sapiens
tcccgatggcttttaccctaagtaacttggtatgccatataatatgtaacagcaccaacaggcagagaatcgccagaaaacactcttgattacctcaaacgaaaaagtaccaccaggatc

SEQ ID NO: 1134 - DNA, 120 bp, Homo sapiens
tgctgaatgaacttatctccacgttccctgccctactgacacaaccccctcccaagtttattgttaacttacacattcaatgcacagcacacctttactcaaacaatggaaaagaaagaa

SEQ ID NO: 1135 - DNA, 120 bp, Homo sapiens
agtaaaaacccaattgtttaccttcttaaatcactgcttgaagagcaaatctttccattttgctgaatgaacttatctccacgttccctgccctactgacacaaccccctcccaagttta

SEQ ID NO: 1136 - DNA, 120 bp, Homo sapiens
gggcttaatatgacattgaggtcactagtaatttagctggaaagtctgtaacacagcacttcccgatggcttttaccctaagtaacttggtatgccatataatatgtaacagcaccaaca

SEQ ID NO: 1137 - DNA, 120 bp, Homo sapiens
ctgttcagaagctaattttagtaattaagggaatcatatgctatgttcaaataccatgccagtaaaaacccaattgtttaccttcttaaatcactgcttgaagagcaaatctttccattt

SEQ ID NO: 1138 - DNA, 120 bp, Homo sapiens
ggcagagaatcgccagaaaacactcttgattacctcaaacgaaaaagtaccaccaggatcctgttcagaagctaattttagtaattaagggaatcatatgctatgttcaaataccatgcc

SEQ ID NO: 1139 - DNA, 120 bp, Homo sapiens
agtgtcaattcaaagtggcccttgtctattccttaaggagtagacttccattttcatcagatttggatttagcatagacatattgattaccttgaagaagaattcatataattttatctt

SEQ ID NO: 1140 - DNA, 120 bp, Homo sapiens
ttgttaacttacacattcaatgcacagcacacctttactcaaacaatggaaaagaaagaaagtgtcaattcaaagtggcccttgtctattccttaaggagtagacttccattttcatcag

SEQ ID NO: 1141 - DNA, 120 bp, Homo sapiens
atttggatttagcatagacatattgattaccttgaagaagaattcatataattttatcttctgattcccatcactcaaatcaaaattacataatatattccaaaatggcaactaggaatg

SEQ ID NO: 1142 - DNA, 120 bp, Homo sapiens
tttctttgtagatgattcattcctggctgcatttgaaaaccacatattgttaattgcttgacgaatttaaatcccttgactacttttcatttcagaaaacacttacaaaaaaagtccaaa

SEQ ID NO: 1143 - DNA, 120 bp, Homo sapiens
tgtaaagccatttctcatttttctccgcactttccaagggtacactccttgtttccaagatggaatgagaaataaagaagtgcccttcctgccatcttctcccctgaccctttcctcctt

SEQ ID NO: 1144 - DNA, 120 bp, Homo sapiens
tggaatgagaaataaagaagtgcccttcctgccatcttctcccctgaccctttcctccttcccactttcctcctattcctccccaaacatgatttatttctgcgttttgcaactcttgag

SEQ ID NO: 1145 - DNA, 120 bp, Homo sapiens
agtgctttgatacatcgtaattgtatctacctccattcacacctacttaagatatctgtctaaaagtagactagacagattattcagagagtggagggcagaagggctgtctctgtatct

SEQ ID NO: 1146 - DNA, 120 bp, Homo sapiens
taaaagtagactagacagattattcagagagtggagggcagaagggctgtctctgtatcttaaagaagctggcactcttcagctgatggctgcttggtcttgaggcctcaagatctttaa

SEQ ID NO: 1147 - DNA, 120 bp, Homo sapiens
tgaggaccttccctccagtgaattagctgtggctttctcacagtccatagttaggataaatgtaaagccatttctcatttttctccgcactttccaagggtacactccttgtttccaaga

SEQ ID NO: 1148 - DNA, 120 bp, Homo sapiens
aaggtagtaggcctttcagaaatttcaggtagcagccaaaccccagaagaagagaaggaacacagagacctagaccatgtgagaacctgaggtgtgcagcatttacttcacagattcgtc

SEQ ID NO: 1149 - DNA, 120 bp, Homo sapiens
acgaatttaaatcccttgactacttttcatttcagaaaacacttacaaaaaaagtccaaatgaggaccttccctccagtgaattagctgtggctttctcacagtccatagttaggataaa

SEQ ID NO: 1150 - DNA, 120 bp, Homo sapiens
cacagagacctagaccatgtgagaacctgaggtgtgcagcatttacttcacagattcgtctagcatatttgagaggtgtctttcctactaggagactgaactctgcatctgagaataaaa

SEQ ID NO: 1151 - DNA, 120 bp, Homo sapiens
ttctcagcatttagtaaatggtgttggtccctgttgattccttcctctcctggaccatggaaggtagtaggcctttcagaaatttcaggtagcagccaaaccccagaagaagagaaggaa

SEQ ID NO: 1152 - DNA, 120 bp, Homo sapiens
tagcatatttgagaggtgtctttcctactaggagactgaactctgcatctgagaataaaaacttaacatatctacaggttttgacaacctctgtgaattatctagttgagaggatggctc

SEQ ID NO: 1153 - DNA, 120 bp, Homo sapiens
tttttttctagcagctgttgttgtttctgaaagaatcttgagggtgtttggagtctcagaatggcttccttaaagactaccttcagactctcagctgctcatccacaacagagatcagcc

SEQ ID NO: 1154 - DNA, 120 bp, Homo sapiens
tctggctttctctatagtgtttcattcactgtttggtgatggaatctcttcagttcagagatacttaatagatatagctttttctttcctgcttccaggcctacctacctgtttcttgct

SEQ ID NO: 1155 - DNA, 120 bp, Homo sapiens
atacttaatagatatagctttttctttcctgcttccaggcctacctacctgtttcttgcttttttttctagcagctgttgttgtttctgaaagaatcttgagggtgtttggagtctcaga

SEQ ID NO: 1156 - DNA, 120 bp, Homo sapiens
atggcttccttaaagactaccttcagactctcagctgctcatccacaacagagatcagcctttctttgtagatgattcattcctggctgcatttgaaaaccacatattgttaattgcttg

SEQ ID NO: 1157 - DNA, 120 bp, Homo sapiens
acttaacatatctacaggttttgacaacctctgtgaattatctagttgagaggatggctcaaggagcctattgccatggtctgatgtcgttatggacgctatgaacatccttgcagtttc

SEQ ID NO: 1158 - DNA, 120 bp, Homo sapiens
tttcagccctttcttagattgtctgttccctgctcccagaagtatagatagtgagacttgagtgctttgatacatcgtaattgtatctacctccattcacacctacttaagatatctgtc

SEQ ID NO: 1159 - DNA, 120 bp, Homo sapiens
taaagaagctggcactcttcagctgatggctgcttggtcttgaggcctcaagatctttaatctggctttctctatagtgtttcattcactgtttggtgatggaatctcttcagttcagag

SEQ ID NO: 1160 - DNA, 120 bp, Homo sapiens
cccactttcctcctattcctccccaaacatgatttatttctgcgttttgcaactcttgagttctcagcatttagtaaatggtgttggtccctgttgattccttcctctcctggaccatgg

SEQ ID NO: 1161 - DNA, 120 bp, Homo sapiens
aaggagcctattgccatggtctgatgtcgttatggacgctatgaacatccttgcagtttccattgttgaagacagccctgatgccagctgtctcatcattccccatgttcaagagcatcc

SEQ ID NO: 1162 - DNA, 120 bp, Homo sapiens
cattgttgaagacagccctgatgccagctgtctcatcattccccatgttcaagagcatcccagcattgctacctcaggatcccatgtcctgaatgcaacagagtgatttcgctgctgaat

SEQ ID NO: 1163 - DNA, 120 bp, Homo sapiens
ggcagcagcacaaatgttttgttgatgagggtttaaattgtagaaagtgagacaattttaggaaggccagctagagagaaatttctagcatcaaattttgctaaacacctaggatttgta

SEQ ID NO: 1164 - DNA, 120 bp, Homo sapiens
cattgaaagcaaacttggttattatactaggtagtttagacttcaagcagttgaaaatctttgagcatgggataggcatgatgacattgtgtttatttgcatgtttctttaaagaaaact

SEQ ID NO: 1165 - DNA, 120 bp, Homo sapiens
gttacctccatttgggttgttacctgcaagtactgaccacgtatatgaagaagtactggtttagaccaaggcaattggcttgtataagaggcctaccctcataccaaaagccagtttcct

SEQ ID NO: 1166 - DNA, 120 bp, Homo sapiens
ttgagcatgggataggcatgatgacattgtgtttatttgcatgtttctttaaagaaaactggcagcagcacaaatgttttgttgatgagggtttaaattgtagaaagtgagacaatttta

SEQ ID NO: 1167 - DNA, 120 bp, Homo sapiens
ggaaggccagctagagagaaatttctagcatcaaattttgctaaacacctaggatttgtagttacctccatttgggttgttacctgcaagtactgaccacgtatatgaagaagtactggt

SEQ ID NO: 1168 - DNA, 120 bp, Homo sapiens
agtgggaagtctcatttctgagaagtgctgcttcctacccacaattctttgatagctgagtgctttagctgatctgcataactgaggtgtgcaccaaggagcagaattactctataaatt

SEQ ID NO: 1169 - DNA, 120 bp, Homo sapiens
tgcccaagttatctaatgtctgcaggtacatattcctggcctaaggattgtgctaaagaagttatttctaagaaatatagtgacttccagcatcatgcagaatgaccatttaatattttg

SEQ ID NO: 1170 - DNA, 120 bp, Homo sapiens
tgctttagctgatctgcataactgaggtgtgcaccaaggagcagaattactctataaattttggcatcaacatgtgcaacttgtgactcagcactttgaaactctggggatttttttgtt

SEQ ID NO: 1171 - DNA, 120 bp, Homo sapiens
taagaatcagtcaataatgcattaatgatcaaaagcagaccatccttaccacatggtgcataagattatgctattatgctattagctactaatgccactaaagttaattatgttgggtct

SEQ ID NO: 1172 - DNA, 120 bp, Homo sapiens
taagattatgctattatgctattagctactaatgccactaaagttaattatgttgggtctgcaacgttgtcatacacaaaggataggatgcaaaactgtcctaggccaaagcatggttat

SEQ ID NO: 1173 - DNA, 120 bp, Homo sapiens
gcaacgttgtcatacacaaaggataggatgcaaaactgtcctaggccaaagcatggttattgcccaagttatctaatgtctgcaggtacatattcctggcctaaggattgtgctaaagaa

SEQ ID NO: 1174 - DNA, 120 bp, Homo sapiens
aatatctagacattctgctgtagaatttaatagtccttttatacactgtctgaccaacattttgacatttactcagaaccccatcacagtgctaccacataacctcattgctaaagtggg

SEQ ID NO: 1175 - DNA, 120 bp, Homo sapiens
cttccattctcctttgattctctagactagaattccaaagaccctcaggctggtgatgcaagtgggaagtctcatttctgagaagtgctgcttcctacccacaattctttgatagctgag

SEQ ID NO: 1176 - DNA, 120 bp, Homo sapiens
ataggcagagaatacctatacctctagctcaggtcatcattttgcagatgtgtgtgtcattaagaatcagtcaataatgcattaatgatcaaaagcagaccatccttaccacatggtgca

SEQ ID NO: 1177 - DNA, 120 bp, Homo sapiens
ttcagcaggcagcagagtgtcataaagaattaacaacgtggaactcagttactgggatttcttccattctcctttgattctctagactagaattccaaagaccctcaggctggtgatgca

SEQ ID NO: 1178 - DNA, 120 bp, Homo sapiens
aggcctagaaatcacagatttgtagaaaccatccaatgattgaatcccctctacttcctgttcagcaggcagcagagtgtcataaagaattaacaacgtggaactcagttactgggattt

SEQ ID NO: 1179 - DNA, 120 bp, Homo sapiens
tttgacatttactcagaaccccatcacagtgctaccacataacctcattgctaaagtgggaggcctagaaatcacagatttgtagaaaccatccaatgattgaatcccctctacttcctg

SEQ ID NO: 1180 - DNA, 120 bp, Homo sapiens
gttatttctaagaaatatagtgacttccagcatcatgcagaatgaccatttaatattttgaatatctagacattctgctgtagaatttaatagtccttttatacactgtctgaccaacat

SEQ ID NO: 1181 - DNA, 120 bp, Homo sapiens
ttggtctgtaaattacccttgaaacaacccttgaaatttccactccatgacctaaatcgtcatccctaaattggttacatacatatttggtgacactttggaggggaaaagctttatgtc

SEQ ID NO: 1182 - DNA, 120 bp, Homo sapiens
taccgcatatctgatacttgaatgagtacctccttgtaaaatttatacttaaatccttgagtttttaaagtgtaatagcaatagaaagattttattgttgtttacttttactgtgagtgc

SEQ ID NO: 1183 - DNA, 120 bp, Homo sapiens
attcctccaaaccaaattatctgggatagcacatatatgttgtactctgtctctgagcatttgctcttagagaactatggttagagcgaagtaaatttttctaatcataaaaattaatga

SEQ ID NO: 1184 - DNA, 120 bp, Homo sapiens
catccctaaattggttacatacatatttggtgacactttggaggggaaaagctttatgtctctctaactgtagttcttaagggaatttgcatatggaaaaaacagagactgcgtctctta

SEQ ID NO: 1185 - DNA, 120 bp, Homo sapiens
ttgctcttagagaactatggttagagcgaagtaaatttttctaatcataaaaattaatgataccgcatatctgatacttgaatgagtacctccttgtaaaatttatacttaaatccttga

SEQ ID NO: 1186 - DNA, 120 bp, Homo sapiens
aggtagtaggcagaaaactgagtacacagcacacaataggccatatatacaaaagcaagtattttgcaaataataataattcaggaaaaaagcttcactttcgttggtaacctgtttgtt

SEQ ID NO: 1187 - DNA, 120 bp, Homo sapiens
ggtctgttctctctctctctgctgttacagtgtggcttttggttcttgtttctttgttctttggtctgtaaattacccttgaaacaacccttgaaatttccactccatgacctaaatcgt

SEQ ID NO: 1188 - DNA, 120 bp, Homo sapiens
tccaaaatccctcagttgctcttgaaagagcaagatgatgccataggcaatattttccaaaggtagtaggcagaaaactgagtacacagcacacaataggccatatatacaaaagcaagt

SEQ ID NO: 1189 - DNA, 120 bp, Homo sapiens
tctctaactgtagttcttaagggaatttgcatatggaaaaaacagagactgcgtctcttaattcctccaaaccaaattatctgggatagcacatatatgttgtactctgtctctgagcat

SEQ ID NO: 1190 - DNA, 120 bp, Homo sapiens
gtttttaaagtgtaatagcaatagaaagattttattgttgtttacttttactgtgagtgctccaaaatccctcagttgctcttgaaagagcaagatgatgccataggcaatattttccaa

SEQ ID NO: 1191 - DNA, 120 bp, Homo sapiens
tacccttcagcccttcttttgtggatgaaaaatggtctttgtgctcccagtcaccactggggtctgttctctctctctctgctgttacagtgtggcttttggttcttgtttctttgttct

SEQ ID NO: 1192 - DNA, 120 bp, Homo sapiens
tcttacaagtggataggaaaagaaaaacccccagtaaaaaatggcaaccgcccacctccccaactttacatgctgcttcctatgttagaggatctgtcttaggcatctgattatggagcc

SEQ ID NO: 1193 - DNA, 120 bp, Homo sapiens
aacacgcagccacccagaaatcggtagagccttcagctcatctgttattaatatttctgtgacaacagatatctaggaagtaaacaggaaattgcatcgctatcctgcatcacctttttt

SEQ ID NO: 1194 - DNA, 120 bp, Homo sapiens
ctcttctcctacatcttctcactccccaccccacccccacatacacacaattcttgtccactatgttcagagagatgcacgcacacatatatatgtatatatatagtatatttgtcaata

SEQ ID NO: 1195 - DNA, 120 bp, Homo sapiens
gacaacagatatctaggaagtaaacaggaaattgcatcgctatcctgcatcaccttttttggaatcaggttccattcttctcagtccagttcaaccttgtgatactttttagatctcaac

SEQ ID NO: 1196 - DNA, 120 bp, Homo sapiens
gctttgattagtgagctgttaggcacacagacattaatttcaaagcattctcatctccagtctgagtaataatgcttatagtattatgcaattgtttggctgctgcaagaaattcagcag

SEQ ID NO: 1197 - DNA, 120 bp, Homo sapiens
ggaatcaggttccattcttctcagtccagttcaaccttgtgatactttttagatctcaaccaaggcatagaaatatattttcccttgcttaataccccatggaaccaatgcccctgtggt

SEQ ID NO: 1198 - DNA, 120 bp, Homo sapiens
gccacaatgaaactgaaagagactgatgactctcctcagggtggaaaatgaggcatggaagctttgattagtgagctgttaggcacacagacattaatttcaaagcattctcatctccag

SEQ ID NO: 1199 - DNA, 120 bp, Homo sapiens
atgtaagcaccgagcacctgcagaaaacttggactggcatttggatctaagaagaaaatctgcatcttgaccaagatgaaaagtcaccagcccaagcttgtgcagtgaagtgtcatgttg

SEQ ID NO: 1200 - DNA, 120 bp, Homo sapiens
ttttggagacatttgcacatcttttgggatcacgttgttaagaagtagaactaagggaaaaacacgcagccacccagaaatcggtagagccttcagctcatctgttattaatatttctgt

SEQ ID NO: 1201 - DNA, 120 bp, Homo sapiens
gcgtctgaggcttaggagcttaggtttttgctcctcaacacagactttgacgttggggttgggggctactctcttgattgctgactccctccagcgggaccaatagtgttttcctacctc

SEQ ID NO: 1202 - DNA, 120 bp, Homo sapiens
caactttacatgctgcttcctatgttagaggatctgtcttaggcatctgattatggagcctgctagatacaagcccgtatttagactgctacagtcaacaatgtctctctttcatactag

SEQ ID NO: 1203 - DNA, 120 bp, Homo sapiens
aaaaattccgggttggcaattgcaagcatctcaaaatgaccagaccctgaagaaaggctgacttgcctcattcaaaatgagggctctagagggctctagtggatagtctggagaaacctg

SEQ ID NO: 1204 - DNA, 120 bp, Homo sapiens
atttaaaaagagtgtcacttgttacagattgtgggatgtgttccttaagatcacaaaaatgtaaaatattttctttttatactgaacacatgcatagacaacttacctgagcaagctgct

SEQ ID NO: 1205 - DNA, 120 bp, Homo sapiens
tgaagtaaaaattgattgttgagggacatttcagccctctagcagtcaacaattaaaaacatgtaagcaccgagcacctgcagaaaacttggactggcatttggatctaagaagaaaatc

SEQ ID NO: 1206 - DNA, 120 bp, Homo sapiens
tgctagatacaagcccgtatttagactgctacagtcaacaatgtctctctttcatactagaaaaattccgggttggcaattgcaagcatctcaaaatgaccagaccctgaagaaaggctg

SEQ ID NO: 1207 - DNA, 120 bp, Homo sapiens
gtaaaatattttctttttatactgaacacatgcatagacaacttacctgagcaagctgctttttggagacatttgcacatcttttgggatcacgttgttaagaagtagaactaagggaaa

SEQ ID NO: 1208 - DNA, 120 bp, Homo sapiens
tctgagtaataatgcttatagtattatgcaattgtttggctgctgcaagaaattcagcagactccaacaagtagtctttcttggtctctgagtgactgtaacttaaattctacctccctt

SEQ ID NO: 1209 - DNA, 120 bp, Homo sapiens
ctatgttcagagagatgcacgcacacatatatatgtatatatatagtatatttgtcaataaagcagaaaagaagaaaaaactccaagtaaacaattttccatttccccatctcacttctg

SEQ ID NO: 1210 - DNA, 120 bp, Homo sapiens
actccaacaagtagtctttcttggtctctgagtgactgtaacttaaattctacctcccttctcttctcctacatcttctcactccccaccccacccccacatacacacaattcttgtcca

SEQ ID NO: 1211 - DNA, 120 bp, Homo sapiens
tgcatcttgaccaagatgaaaagtcaccagcccaagcttgtgcagtgaagtgtcatgttggccacaatgaaactgaaagagactgatgactctcctcagggtggaaaatgaggcatggaa

SEQ ID NO: 1212 - DNA, 120 bp, Homo sapiens
aagcagaaaagaagaaaaaactccaagtaaacaattttccatttccccatctcacttctgtcttacaagtggataggaaaagaaaaacccccagtaaaaaatggcaaccgcccacctccc

SEQ ID NO: 1213 - DNA, 120 bp, Homo sapiens
acttgcctcattcaaaatgagggctctagagggctctagtggatagtctggagaaacctggcgtctgaggcttaggagcttaggtttttgctcctcaacacagactttgacgttggggtt

SEQ ID NO: 1214 - DNA, 120 bp, Homo sapiens
caaggcatagaaatatattttcccttgcttaataccccatggaaccaatgcccctgtggttgaagtaaaaattgattgttgagggacatttcagccctctagcagtcaacaattaaaaac

SEQ ID NO: 1215 - DNA, 120 bp, Homo sapiens
actccttgattgctctctcacatcacatgcttctcttcatcagttgtaagcctctcattcttctcccaagccagactcaaatattgtattgatgtcaaagaagaatcacttagagtttgg

SEQ ID NO: 1216 - DNA, 120 bp, Homo sapiens
aaacacactgagagactacagtccgactttccctcttacatctagccttactgtagccacactccttgattgctctctcacatcacatgcttctcttcatcagttgtaagcctctcattc

SEQ ID NO: 1217 - DNA, 120 bp, Homo sapiens
agtaaacaaggaccagatttctgcttctccaggagaagaagccagccaacccctctcttcaaacacactgagagactacagtccgactttccctcttacatctagccttactgtagccac

SEQ ID NO: 1218 - DNA, 120 bp, Homo sapiens
tagatcttagcctcaggccctgtcactgagctgaaggtagtagctgatccacagaagttcagtaaacaaggaccagatttctgcttctccaggagaagaagccagccaacccctctcttc

SEQ ID NO: 1219 - DNA, 120 bp, Homo sapiens
ttctcccaagccagactcaaatattgtattgatgtcaaagaagaatcacttagagtttggaatatcttgttctctctctgctccatagcttccatattgacaccagtttctttctagtgg

SEQ ID NO: 1220 - DNA, 120 bp, Homo sapiens
cttgatcacatatgatgggggccaggcactgactcaggcggatgcagtgaagctctggctcagtcgcttgcttttcgtggtgtgctgccaggaagaaactttgctgatgggactcaaggt

SEQ ID NO: 1221 - DNA, 120 bp, Homo sapiens
tatcatgattattgtttcctgtaatgtggcttggcattggcaaagtgctttttgattgttcttgatcacatatgatgggggccaggcactgactcaggcggatgcagtgaagctctggct

SEQ ID NO: 1222 - DNA, 120 bp, Homo sapiens
agggatgttgtgaggacgggctgtagaagtaatagtggttaccattcatgtagttgtgagtatcatgattattgtttcctgtaatgtggcttggcattggcaaagtgctttttgattgtt

SEQ ID NO: 1223 - DNA, 120 bp, Homo sapiens
cagtcgcttgcttttcgtggtgtgctgccaggaagaaactttgctgatgggactcaaggtgtcaccttggacaagaagcaactgtgtctgtctgaggttcctgtggccatctttatttgt

SEQ ID NO: 1224 - DNA, 120 bp, Homo sapiens
gtcaccttggacaagaagcaactgtgtctgtctgaggttcctgtggccatctttatttgtgtattaggcaattcgtatttcccccttaggttctagccttctggatcccagccagtgacc

SEQ ID NO: 1225 - DNA, 120 bp, Homo sapiens
agaagtggagtctgtgaagccagggaaacacacatgtgagagtcagaaggactctccctgacttgcctggggcctgtctttcccaccttctccagtctgtctaaacacacacacacacac

SEQ ID NO: 1226 - DNA, 120 bp, Homo sapiens
gtattaggcaattcgtatttcccccttaggttctagccttctggatcccagccagtgacctagatcttagcctcaggccctgtcactgagctgaaggtagtagctgatccacagaagttc

SEQ ID NO: 1227 - DNA, 120 bp, Homo sapiens
aatatcttgttctctctctgctccatagcttccatattgacaccagtttctttctagtggagaagtggagtctgtgaagccagggaaacacacatgtgagagtcagaaggactctccctg

SEQ ID NO: 1228 - DNA, 120 bp, Homo sapiens
ttttaactactttctatttgtttgaatgttgcatatttctactagtgaaattttcccttaataaagccattaatacaccaatcgtattttcttatttacaacagactgagagaattaatg

SEQ ID NO: 1229 - DNA, 120 bp, Homo sapiens
tctggagaaaagccaaggaaggacttcaggaggggagtttcccccttctcagggcagaattttaatctccagaccaacaagaagttccctaatgtggattgaaaggctaatgaggtttat

SEQ ID NO: 1230 - DNA, 120 bp, Homo sapiens
tttaatctccagaccaacaagaagttccctaatgtggattgaaaggctaatgaggtttatttttaactactttctatttgtttgaatgttgcatatttctactagtgaaattttccctta

SEQ ID NO: 1231 - DNA, 120 bp, Homo sapiens
gtatctggacagtgagcccctttcttctggctcagtagtcagagagaggagacttggagacagtttctgctggatcctgtgctttggcaaggatgtgcagcattgcatatcattctatca

SEQ ID NO: 1232 - DNA, 120 bp, Homo sapiens
ccaaggattggctctgctccctgcttctcatccctgtcctagttcttcctcacctatctccatttcccactactgatccttctctccagtaagatgctattcaacccgatgaaatataaa

SEQ ID NO: 1233 - DNA, 120 bp, Homo sapiens
ggctctgcaatttcatatgagcaggtttttggtaaaatcttttgtccctcactcagggtggtatctggacagtgagcccctttcttctggctcagtagtcagagagaggagacttggaga

SEQ ID NO: 1234 - DNA, 120 bp, Homo sapiens
tgggaggggattggagacttgggggaaagaatcaaggagccttcttgcctgggggaatttggcatgcacttattaatcccatttggttgcactccctactaatccctcactccatacctg

SEQ ID NO: 1235 - DNA, 120 bp, Homo sapiens
ttaattatgtttactcctccatgaactaaaaaccattagactaaatagtccaacataaaccttgaaagataaaatttgatattcttttgcctggccatttctctgacccagaattggggc

SEQ ID NO: 1236 - DNA, 120 bp, Homo sapiens
ggcatgcacttattaatcccatttggttgcactccctactaatccctcactccatacctgccaaggattggctctgctccctgcttctcatccctgtcctagttcttcctcacctatctc

SEQ ID NO: 1237 - DNA, 120 bp, Homo sapiens
ttggactgtttcacatttgtttttaatgtcagtttaaatgtaattgtaaaagcatgtatgctctaaaatcatgtagttacttttttcagtggaaaagcctggtattcgaaagcatttcca

SEQ ID NO: 1238 - DNA, 120 bp, Homo sapiens
cagtttctgctggatcctgtgctttggcaaggatgtgcagcattgcatatcattctatcattaattatgtttactcctccatgaactaaaaaccattagactaaatagtccaacataaac

SEQ ID NO: 1239 - DNA, 120 bp, Homo sapiens
ctctaaaatcatgtagttacttttttcagtggaaaagcctggtattcgaaagcatttccaggctctgcaatttcatatgagcaggtttttggtaaaatcttttgtccctcactcagggtg

SEQ ID NO: 1240 - DNA, 120 bp, Homo sapiens
cttgaaagataaaatttgatattcttttgcctggccatttctctgacccagaattggggctgggaggggattggagacttgggggaaagaatcaaggagccttcttgcctgggggaattt

SEQ ID NO: 1241 - DNA, 120 bp, Homo sapiens
cagaaagttcacctgcttggggtaaaggtcatgaagtggagaatgtggggctcagtaactagcaatagtaaaaaacatcattgattggcttgcagaatttactctgttctaagcatctta

SEQ ID NO: 1242 - DNA, 120 bp, Homo sapiens
ctctcttatgttacatttcagtccttgtgaaactctatatgtttcatcagttcactttttcagaaagttcacctgcttggggtaaaggtcatgaagtggagaatgtggggctcagtaact

SEQ ID NO: 1243 - DNA, 120 bp, Homo sapiens
ctctcaggaaccctcgaaaaaacttataggacttataggactgttggggatctgccaagtctctcttatgttacatttcagtccttgtgaaactctatatgtttcatcagttcacttttt

SEQ ID NO: 1244 - DNA, 120 bp, Homo sapiens
ccagcatgactctcaatctttgacttgagaccagttgcccaacatggaaggttatacttttcacagtttaccaccataagcagtctttcagagtgatttctagctagagatccattctta

SEQ ID NO: 1245 - DNA, 120 bp, Homo sapiens
taataaaccttttatgtgaaaagcattttagaacttcagtgtcattattgcattctgcctcctggagttcagtgcactttttcaccatgctttaatcttggagtcctggtggtacagaat

SEQ ID NO: 1246 - DNA, 120 bp, Homo sapiens
gagctcccctgactcacaatccagtattcctctgaccttctaatcctaaagttatacagtaaggtcccttgactctaatcctagtagatggaaagatggctggcatgatttaagccagag

SEQ ID NO: 1247 - DNA, 120 bp, Homo sapiens
tttagatttaagcattgcaatatataagcactgcacacatgcattcacaaaagtatagcctagtctagcttcacaaagaatttgtagccctacaccaaacacacctttatgtttacttag

SEQ ID NO: 1248 - DNA, 120 bp, Homo sapiens
tagtctagcttcacaaagaatttgtagccctacaccaaacacacctttatgtttacttagtgtttagaattagatttaagatcagaatttagtttcacaggcattcatgtgtggaagaac

SEQ ID NO: 1249 - DNA, 120 bp, Homo sapiens
ccctgtcaatcctgctttctgccattcttcatgcctgagttagggcccctgcaagccattcactggttaatctttaggaatgaatggagagtgaaaaccagtttggagggttcactgtgt

SEQ ID NO: 1250 - DNA, 120 bp, Homo sapiens
tgtttagaattagatttaagatcagaatttagtttcacaggcattcatgtgtggaagaacctcagttattgttttttgtttcatactgtctcacccttgctttccctgctgtgtctggac

SEQ ID NO: 1251 - DNA, 120 bp, Homo sapiens
ctcagttattgttttttgtttcatactgtctcacccttgctttccctgctgtgtctggacccctgtcaatcctgctttctgccattcttcatgcctgagttagggcccctgcaagccatt

SEQ ID NO: 1252 - DNA, 120 bp, Homo sapiens
agagaagccagaacacctggtgcagctagggccactgtggtcacagggacaagcacactacctgggtcctggaggcaagtgggaatgcagtttttcttccttaagcagatgccatatagg

SEQ ID NO: 1253 - DNA, 120 bp, Homo sapiens
ccaggattccaagtcgtctagcaacatcctggtctctgctgcagacagaacagaggatcccccggcagaatgaatggagtctgatttcaattacgttcagtatagtcactctctttaggc

SEQ ID NO: 1254 - DNA, 120 bp, Homo sapiens
atggccctttcttgtcactgctctgacccaagactaacagggcagagatagtgaactcacatactattaaaactatccacttatacttccccctttctctttgctttatcactccattta

SEQ ID NO: 1255 - DNA, 120 bp, Homo sapiens
tatcaggccaggtgttacaatctcctaaagaggaggtatggactggaaagccccttgccaatggccctttcttgtcactgctctgacccaagactaacagggcagagatagtgaactcac

SEQ ID NO: 1256 - DNA, 120 bp, Homo sapiens
caggggtctaatgccccagggctgaaaagttagttccccataggatccatccaggcatgatatcaggccaggtgttacaatctcctaaagaggaggtatggactggaaagccccttgcca

SEQ ID NO: 1257 - DNA, 120 bp, Homo sapiens
agtaaaccaatgagtctctgccttgacacagtggcaagctgacctgtatcttatatgaaagaattagatttgactctggggctcaggtgcagagggcaggaggggcataaggatggcctt

SEQ ID NO: 1258 - DNA, 120 bp, Homo sapiens
cctgggtcctggaggcaagtgggaatgcagtttttcttccttaagcagatgccatataggcctggggaggaggatgtgagaataccagccaagttctcattggcactatacagagaaagg

SEQ ID NO: 1259 - DNA, 120 bp, Homo sapiens
ttttccttaatcatggttgagaaggcctatatcttggagtggccaggagtgagactggaacagtacctaaaggttaaggacgctaaagaagttacagattggttacatctgctcctccct

SEQ ID NO: 1260 - DNA, 120 bp, Homo sapiens
aggaatgatccatggaacctgatttgaaatttttttctctggtgctatagatagctcccacaggggtctaatgccccagggctgaaaagttagttccccataggatccatccaggcatga

SEQ ID NO: 1261 - DNA, 120 bp, Homo sapiens
ggaattatttcatcttgatggattctccccacagtctctgcacatattgatcttacttgtaatgagtttgcttaggttcacgagtcatcatcccagggagatctgagtcattggtgggaa

SEQ ID NO: 1262 - DNA, 120 bp, Homo sapiens
cctggggaggaggatgtgagaataccagccaagttctcattggcactatacagagaaaggggaattatttcatcttgatggattctccccacagtctctgcacatattgatcttacttgt

SEQ ID NO: 1263 - DNA, 120 bp, Homo sapiens
gaccttaggtaaagggattcaaacttcctaagactttggaaacttcacgccactttcaccttttccttaatcatggttgagaaggcctatatcttggagtggccaggagtgagactggaa

SEQ ID NO: 1264 - DNA, 120 bp, Homo sapiens
catggaagaaaagaagtccttggatactgagtaacagctgagactagcaagcctcattgtccaggattccaagtcgtctagcaacatcctggtctctgctgcagacagaacagaggatcc

SEQ ID NO: 1265 - DNA, 120 bp, Homo sapiens
cagtacctaaaggttaaggacgctaaagaagttacagattggttacatctgctcctccctaggaatgatccatggaacctgatttgaaatttttttctctggtgctatagatagctccca

SEQ ID NO: 1266 - DNA, 120 bp, Homo sapiens
aatgagtttgcttaggttcacgagtcatcatcccagggagatctgagtcattggtgggaaagtcgaggcgacagattatatctcactgatctcactgtcaccaattgctctgtgtgtccc

SEQ ID NO: 1267 - DNA, 120 bp, Homo sapiens
gaattagatttgactctggggctcaggtgcagagggcaggaggggcataaggatggccttcatggaagaaaagaagtccttggatactgagtaacagctgagactagcaagcctcattgt

SEQ ID NO: 1268 - DNA, 120 bp, Homo sapiens
agtcgaggcgacagattatatctcactgatctcactgtcaccaattgctctgtgtgtccctccaccttttgaaaaagtccatggattcatttgtgtgtaattcatttggatttatttctt

SEQ ID NO: 1269 - DNA, 120 bp, Homo sapiens
atactattaaaactatccacttatacttccccctttctctttgctttatcactccatttaagtaaaccaatgagtctctgccttgacacagtggcaagctgacctgtatcttatatgaaa

SEQ ID NO: 1270 - DNA, 120 bp, Homo sapiens
tccaccttttgaaaaagtccatggattcatttgtgtgtaattcatttggatttatttcttctttatcaatagctttagtggggtattgcaaatgggaaagttgccccagagaacagtgta

SEQ ID NO: 1271 - DNA, 120 bp, Homo sapiens
cccggcagaatgaatggagtctgatttcaattacgttcagtatagtcactctctttaggcagagaagccagaacacctggtgcagctagggccactgtggtcacagggacaagcacacta

SEQ ID NO: 1272 - DNA, 120 bp, Homo sapiens
gattcttttcatgtgtcaaatttcatagtgagatagggagaacagaaacatcacatccttgaccttaggtaaagggattcaaacttcctaagactttggaaacttcacgccactttcacc

SEQ ID NO: 1273 - DNA, 120 bp, Homo sapiens
attttgggatacacaagtttcaggaattttttaaaaatctattaatgccttctaggtgtgtgtatgcacgcttgcagacatgtgcccatgcacaagcatgggaaggcagtaaggcattca

SEQ ID NO: 1274 - DNA, 120 bp, Homo sapiens
gtttggagtcaagggtgaagaagggaggcagggatgatataaccccagccccactcctcaactctgcttttgagttagaagtagggttcagggcttcagattccttggggaggcagtaga

SEQ ID NO: 1275 - DNA, 120 bp, Homo sapiens
gtcatgctgaaaatctttaagccactatagtgtcccaaatctattccagtttgggcagatgactggagtattctcatagcctcctgtctattcccttctggatttgatactagttatgaa

SEQ ID NO: 1276 - DNA, 120 bp, Homo sapiens
gactggagtattctcatagcctcctgtctattcccttctggatttgatactagttatgaagtttggagtcaagggtgaagaagggaggcagggatgatataaccccagccccactcctca

SEQ ID NO: 1277 - DNA, 120 bp, Homo sapiens
caaagctagaaaggtccagtaatggggaagatggggtctttctgtaggaactgtagcaggggagcagatcctgtaggccaccagtctgtggagctgtgtccaagaactcatgtttgcaat

SEQ ID NO: 1278 - DNA, 120 bp, Homo sapiens
aagcccaccaaatgacaagttattgtggggttcaggcctctaactcaagaagatggtcttggcccagatcataccttgcagcctgtgcctttggtgggatgtgggtgttggcagtggcta

SEQ ID NO: 1279 - DNA, 120 bp, Homo sapiens
tgcatatctccttattactggctgtgccaaagccccgcagaaatgattgttggacaaagtcatcttgcactcagggctggttttccaggcttccttgttattttcccctgagttcttctg

SEQ ID NO: 1280 - DNA, 120 bp, Homo sapiens
ggcccagatcataccttgcagcctgtgcctttggtgggatgtgggtgttggcagtggctatgcatatctccttattactggctgtgccaaagccccgcagaaatgattgttggacaaagt

SEQ ID NO: 1281 - DNA, 120 bp, Homo sapiens
tgttcctcttgcaacaccaaccccactattttcctcttccctaccctagttgttggtccaaacatgtaatccattcttgcagtgatttattgggtgacaccatgactggagtttgcattg

SEQ ID NO: 1282 - DNA, 120 bp, Homo sapiens
catcttgcactcagggctggttttccaggcttccttgttattttcccctgagttcttctgtgttcctcttgcaacaccaaccccactattttcctcttccctaccctagttgttggtcca

SEQ ID NO: 1283 - DNA, 120 bp, Homo sapiens
ggagcagatcctgtaggccaccagtctgtggagctgtgtccaagaactcatgtttgcaataagcccaccaaatgacaagttattgtggggttcaggcctctaactcaagaagatggtctt

SEQ ID NO: 1284 - DNA, 120 bp, Homo sapiens
tggttcatggtctttctcagtgcaactgcttatgctagacctcagaattatgaccttttcaattatttatatttctgtctatataaatactggaaaaaatagtacaaagtaagcatcgga

SEQ ID NO: 1285 - DNA, 120 bp, Homo sapiens
gagttttggattattgtggttgtcttaaataatgttatttctatcattctttccaatgactgtctcctagcatagttcccattttacagactgatggcagaggcagaaagattctctcac

SEQ ID NO: 1286 - DNA, 120 bp, Homo sapiens
atgcctaaggacctctaaattgtgtgtgtgagcacatggggaagatggttcttaaggtttgagttttggattattgtggttgtcttaaataatgttatttctatcattctttccaatgac

SEQ ID NO: 1287 - DNA, 120 bp, Homo sapiens
aaattctctctttggtttctacctttcttatttattattattatgttaaagggattaaagtggttcatggtctttctcagtgcaactgcttatgctagacctcagaattatgaccttttc

SEQ ID NO: 1288 - DNA, 120 bp, Homo sapiens
tgtctcctagcatagttcccattttacagactgatggcagaggcagaaagattctctcacttctttgatactattgaggacttcagcctttcaccgctcttctcccctttgctaaaaaag

SEQ ID NO: 1289 - DNA, 120 bp, Homo sapiens
aggggccagacacagatgagagcaggttttgttgtatttatccatttaaattgagcaataaaattctctctttggtttctacctttcttatttattattattatgttaaagggattaaag

SEQ ID NO: 1290 - DNA, 120 bp, Homo sapiens
aattatttatatttctgtctatataaatactggaaaaaatagtacaaagtaagcatcggaatgcctaaggacctctaaattgtgtgtgtgagcacatggggaagatggttcttaaggttt

SEQ ID NO: 1291 - DNA, 120 bp, Homo sapiens
cccaaagaacaagtctagtcattctccatttctagtctctttccctagcaatcggctagacatgctagacatagacacatgtacatcactcctttgaattacaacattcagtatttgtct

SEQ ID NO: 1292 - DNA, 120 bp, Homo sapiens
gaaggctatgaatgtcagcccatctttctgaatgtcctggaagccattgagccaggtgtagtgtgtgctggacacgacaacaaccagcccgactcctttgcagccttgctctctagcctc

SEQ ID NO: 1293 - DNA, 120 bp, Homo sapiens
taggttggtctagcacaagaatcagagttttcctctgcaagctatgaaaaatttgggtttagcaggtatttgggatgatgaattatacatttaaccagtgttgaatgagcacttgtcctt

SEQ ID NO: 1294 - DNA, 120 bp, Homo sapiens
taaaaaggtagttgcattgtgtgtttttgaccactgatgataaattcaagtctctcttccttcccaatagcccggaagctgaagaaacttggtaatctgaaactacaggaggaaggagag

SEQ ID NO: 1295 - DNA, 120 bp, Homo sapiens
gtgtgtgctggacacgacaacaaccagcccgactcctttgcagccttgctctctagcctcaatgaactgggagagagacagcttgtacacgtggtcaagtgggccaaggccttgcctggt

SEQ ID NO: 1296 - DNA, 120 bp, Homo sapiens
aaagttccaggtatgaatactgaaggctgcattcaggcagagctggatccaaggatatgctaggttggtctagcacaagaatcagagttttcctctgcaagctatgaaaaatttgggttt

SEQ ID NO: 1297 - DNA, 120 bp, Homo sapiens
gcttccagcaccaccagccccactgaggagacaacccagaagctgacagtgtcacacattgaaggctatgaatgtcagcccatctttctgaatgtcctggaagccattgagccaggtgta

SEQ ID NO: 1298 - DNA, 120 bp, Homo sapiens
ttcccaatagcccggaagctgaagaaacttggtaatctgaaactacaggaggaaggagaggcttccagcaccaccagccccactgaggagacaacccagaagctgacagtgtcacacatt

SEQ ID NO: 1299 - DNA, 120 bp, Homo sapiens
aaggagtttagagtctgtgaccagggagaatggtgattttcttagctagggcagtttttctaaaaaggtagttgcattgtgtgtttttgaccactgatgataaattcaagtctctcttcc

SEQ ID NO: 1300 - DNA, 120 bp, Homo sapiens
agcaggtatttgggatgatgaattatacatttaaccagtgttgaatgagcacttgtccttaaggagtttagagtctgtgaccagggagaatggtgattttcttagctagggcagtttttc

SEQ ID NO: 1301 - DNA, 120 bp, Homo sapiens
tacccatatatgccttaggatgctcttctatatttgcacacacaggctcaccccaaagataatctctagtttgactgacattctgtcttcaatgtcatctttaggagctatatcatggga

SEQ ID NO: 1302 - DNA, 120 bp, Homo sapiens
agaaagatagtattatcgggtgtcttatgtggcccacattgatgcacagcagtcatgctttcatattcaactcacaaaaatggtcagcaaattttccattaatcacaaatcacatagaca

SEQ ID NO: 1303 - DNA, 120 bp, Homo sapiens
tcatattcaactcacaaaaatggtcagcaaattttccattaatcacaaatcacatagacatacccatatatgccttaggatgctcttctatatttgcacacacaggctcaccccaaagat

SEQ ID NO: 1304 - DNA, 120 bp, Homo sapiens
aaattgatggtttccagtttttgaaaaaaggaacgctttttgcaccttaaactacctaaggaatcataatgagaggaaagattaggtaatagtgaaagaattaccaagtgttggtctaac

SEQ ID NO: 1305 - DNA, 120 bp, Homo sapiens
agtgaagtatatgccctttaaaggttctcctaatcctgcaattatgattcaaagattcttttgaaataacaacaaccaaaccttctcttgtggagtcaaagattaacctgcctttcaata

SEQ ID NO: 1306 - DNA, 120 bp, Homo sapiens
ggtcgactagccaataagaacactgggaaggaaacccaaggactctgactggatatgctctgtgccaaaacagagggttcactcagagaggaaaaatataaaaaagaaaaaggagaaggt

SEQ ID NO: 1307 - DNA, 120 bp, Homo sapiens
ggcttgttgcatttgaagacatttcatgttggctgtcaagtcttagatttgtatttccaactcacagggcctggtcacagccctaaccatctcttataccttctcagcttgggaagctga

SEQ ID NO: 1308 - DNA, 120 bp, Homo sapiens
gctctgggctttacagtgaggggccaaggcttcattgaaggccacttgggtcatagtatgggcttgttgcatttgaagacatttcatgttggctgtcaagtcttagatttgtatttccaa

SEQ ID NO: 1309 - DNA, 120 bp, Homo sapiens
ctcacagggcctggtcacagccctaaccatctcttataccttctcagcttgggaagctgaggtcgactagccaataagaacactgggaaggaaacccaaggactctgactggatatgctc

SEQ ID NO: 1310 - DNA, 120 bp, Homo sapiens
tgtgccaaaacagagggttcactcagagaggaaaaatataaaaaagaaaaaggagaaggttgctttaattcttatcactttttcatctggatattttgatatcatgtgtttgacagagat

SEQ ID NO: 1311 - DNA, 120 bp, Homo sapiens
gctttccttggcccaaccaccaagatggaaagccccctccccttacattaacaaatctgcaagccaatatcagttcaccatctagcttgccagactaaatgatttctgaccccaagtctt

SEQ ID NO: 1312 - DNA, 120 bp, Homo sapiens
cttgacaataacctagtgcaaaacactatagcagaatttgtatgacttgggatcactggggctttccttggcccaaccaccaagatggaaagccccctccccttacattaacaaatctgc

SEQ ID NO: 1313 - DNA, 120 bp, Homo sapiens
ttaaaagaatagcttcaaaagaaagccaattaccacattcacaagaactgttcttcatattatctataattacctacaagtacaagtaatttgctaattcaatagattgagttcttgacc

SEQ ID NO: 1314 - DNA, 120 bp, Homo sapiens
tatctataattacctacaagtacaagtaatttgctaattcaatagattgagttcttgacctgtaagatgaactgtgctaggcccctaataagataaattttgttttaagttttctgtgac

SEQ ID NO: 1315 - DNA, 120 bp, Homo sapiens
aagccaatatcagttcaccatctagcttgccagactaaatgatttctgaccccaagtcttttaaaagaatagcttcaaaagaaagccaattaccacattcacaagaactgttcttcatat

SEQ ID NO: 1316 - DNA, 120 bp, Homo sapiens
gtatggacaatttgcttgatacctgtacaatttaattctcatccttccatgtgccttcacattcacacattccaccagaagaccaaggttcaccagccaaaagcttttcttgctccccac

SEQ ID NO: 1317 - DNA, 120 bp, Homo sapiens
atgagggactctagaatccctgatacctggaaggcctaggatctaaaagaaaagaacagggaaatggggctatatgagtggacagggaccaaccaagcagaacaatgtgtctggataatg

SEQ ID NO: 1318 - DNA, 120 bp, Homo sapiens
aatgctaagccactgctttcacgaaactcaattttagctaccacttgccttgcctagaagctcatgcatggaccccaaggtgaaattgtgttctctgaagacctcggctggcagatgtac

SEQ ID NO: 1319 - DNA, 120 bp, Homo sapiens
ctcatgcatggaccccaaggtgaaattgtgttctctgaagacctcggctggcagatgtactacagcagcaaagatttccaaactggcctttctttgagcccattctcccagactagacag

SEQ ID NO: 1320 - DNA, 120 bp, Homo sapiens
ttgagtgctaagcagctctcattaaggacggttaattaatattatggccaaattaagctttcccttttctctcctctttgttagttcggtggcattttagggagaaaaaaataagcatca

SEQ ID NO: 1321 - DNA, 120 bp, Homo sapiens
gttgctacatgcttagaccctgcttcttatttcctgctgagaagggtcagtccaaggcattctgtgctacagaagggttccaagcaggaactactctgggatctgaggctccagccggtc

SEQ ID NO: 1322 - DNA, 120 bp, Homo sapiens
gatagtcatgtaggctacttcagagattgggcattagagaacagagtcaggtattataatcagattagactctagggaggttagccagccatattgctgatatgtgcacagttactgggt

SEQ ID NO: 1323 - DNA, 120 bp, Homo sapiens
cagattagactctagggaggttagccagccatattgctgatatgtgcacagttactgggtttgagtgctaagcagctctcattaaggacggttaattaatattatggccaaattaagctt

SEQ ID NO: 1324 - DNA, 120 bp, Homo sapiens
gaaatggggctatatgagtggacagggaccaaccaagcagaacaatgtgtctggataatgtagacttcagacctgatcctatggctgacaaaagctggtgaccttggtagttcctgagct

SEQ ID NO: 1325 - DNA, 120 bp, Homo sapiens
tctgtgctacagaagggttccaagcaggaactactctgggatctgaggctccagccggtctgtcagcgtgtcattacagtgaaggtgggaagcacaggcctgggagctaagactgctaag

SEQ ID NO: 1326 - DNA, 120 bp, Homo sapiens
tgcctcctacccaagatattcagggtcaacctcccaggcctcttctctaagagatccttggttgctacatgcttagaccctgcttcttatttcctgctgagaagggtcagtccaaggcat

SEQ ID NO: 1327 - DNA, 120 bp, Homo sapiens
aaacagagtctcaaagaaactgcttctgctccctagcgtgtttaatgtgtttcagaacctgagaatgactcctctctgtttctccagaacagcctaacacagtggcaaatgggtgttgag

SEQ ID NO: 1328 - DNA, 120 bp, Homo sapiens
attcacacattccaccagaagaccaaggttcaccagccaaaagcttttcttgctccccactgcctcctacccaagatattcagggtcaacctcccaggcctcttctctaagagatccttg

SEQ ID NO: 1329 - DNA, 120 bp, Homo sapiens
gagactacaagtttctgctgcacatgaaaaaaatatgatgtcaatcggattctagtgagaaaacagagtctcaaagaaactgcttctgctccctagcgtgtttaatgtgtttcagaacct

SEQ ID NO: 1330 - DNA, 120 bp, Homo sapiens
tgtcagcgtgtcattacagtgaaggtgggaagcacaggcctgggagctaagactgctaagatgagggactctagaatccctgatacctggaaggcctaggatctaaaagaaaagaacagg

SEQ ID NO: 1331 - DNA, 120 bp, Homo sapiens
gagaatgactcctctctgtttctccagaacagcctaacacagtggcaaatgggtgttgagtgaatgcatacttaaggaaatctgtagggttgcagctactctttcctcaagtaatccctt

SEQ ID NO: 1332 - DNA, 120 bp, Homo sapiens
tacagcagcaaagatttccaaactggcctttctttgagcccattctcccagactagacaggagactacaagtttctgctgcacatgaaaaaaatatgatgtcaatcggattctagtgaga

SEQ ID NO: 1333 - DNA, 120 bp, Homo sapiens
tgaatgcatacttaaggaaatctgtagggttgcagctactctttcctcaagtaatcccttgatagtcatgtaggctacttcagagattgggcattagagaacagagtcaggtattataat

SEQ ID NO: 1334 - DNA, 120 bp, Homo sapiens
tcccttttctctcctctttgttagttcggtggcattttagggagaaaaaaataagcatcagtatggacaatttgcttgatacctgtacaatttaattctcatccttccatgtgccttcac

SEQ ID NO: 1335 - DNA, 120 bp, Homo sapiens
ggtgatgcctttataactttaagcatccaactgtttcaaaaactccaggagaacatggccatgtctgttctacctgtgtattattgtagacgtagcttctgggagcctctgctctctgag

SEQ ID NO: 1336 - DNA, 120 bp, Homo sapiens
tttcagataattctggatttgttggtgagaagagagagtgttggtagggacgagctctgaggtgatgcctttataactttaagcatccaactgtttcaaaaactccaggagaacatggcc

SEQ ID NO: 1337 - DNA, 120 bp, Homo sapiens
gaaagtgagaagtttgatctaaatttggggaagcattcctaatgaggtatgatgacaaaatttcagataattctggatttgttggtgagaagagagagtgttggtagggacgagctctga

SEQ ID NO: 1338 - DNA, 120 bp, Homo sapiens
agggtgggaaattagtagggccagggtacctattgagtagaaaagaatggagaggaaatgccaggcagaaagaggatggacgcaagagagggaacatgaaagtggtgaacaggtggcagt

SEQ ID NO: 1339 - DNA, 120 bp, Homo sapiens
ccaggcagaaagaggatggacgcaagagagggaacatgaaagtggtgaacaggtggcagtggctgtcaagacatctctccataccctgtacactgtatgtaatatccatctcccagggtt

SEQ ID NO: 1340 - DNA, 120 bp, Homo sapiens
taggcaggtcataaagggcctattcatgtataatgatggcagtaagatgaggatggcagtagggtgggaaattagtagggccagggtacctattgagtagaaaagaatggagaggaaatg

SEQ ID NO: 1341 - DNA, 120 bp, Homo sapiens
tgccctggctttcagccaactggcaggagcccaggaggatggtgctgagaccacccctttcacacccaagaaccaatcctagtcatatttctggtctgctttgcagcttatctcaaaacc

SEQ ID NO: 1342 - DNA, 120 bp, Homo sapiens
catggtgtttgccatgggctggcgatccttcaccaatgtcaactccaggatgctctacttcgcccctgatctggttttcaatgagtaagtgctcctggggcccagacctcactaaaatac

SEQ ID NO: 1343 - DNA, 120 bp, Homo sapiens
acatggaaagattcctccccttcacatataaaagaggcagaaagactctggctttaagggctggagtttcttgggttcttttgctaccaccaaaggctacttctagtcaccatttgctga

SEQ ID NO: 1344 - DNA, 120 bp, Homo sapiens
atttgatctgcagttgtcgcagcggatgccccagccagccaatccagtatgaggcggctttgccctggctttcagccaactggcaggagcccaggaggatggtgctgagaccaccccttt

SEQ ID NO: 1345 - DNA, 120 bp, Homo sapiens
ttatcctcaccatatagtttgtgcttttccccaccaccccttaatggccagcctggatggtccctggggatccttaggggatgcccgaataccagagcatctctgcccaacagggactca

SEQ ID NO: 1346 - DNA, 120 bp, Homo sapiens
cacacccaagaaccaatcctagtcatatttctggtctgctttgcagcttatctcaaaaccacatggaaagattcctccccttcacatataaaagaggcagaaagactctggctttaaggg

SEQ ID NO: 1347 - DNA, 120 bp, Homo sapiens
agcagcttggccagacctggttggtggtgatggtgatggggtgacagtgaagcttagctcatttgatctgcagttgtcgcagcggatgccccagccagccaatccagtatgaggcggctt

SEQ ID NO: 1348 - DNA, 120 bp, Homo sapiens
cgcccctgatctggttttcaatgagtaagtgctcctggggcccagacctcactaaaatacagcagcttggccagacctggttggtggtgatggtgatggggtgacagtgaagcttagctc

SEQ ID NO: 1349 - DNA, 120 bp, Homo sapiens
cttccgcaacttacacgtggacgaccagatggctgtcattcagtactcctggatggggctcatggtgtttgccatgggctggcgatccttcaccaatgtcaactccaggatgctctactt

SEQ ID NO: 1350 - DNA, 120 bp, Homo sapiens
gacttagctcaacccgtcagtacccagactgaccactgcctctgcctcttcttctccaggcttccgcaacttacacgtggacgaccagatggctgtcattcagtactcctggatggggct

SEQ ID NO: 1351 - DNA, 120 bp, Homo sapiens
tccctggggatccttaggggatgcccgaataccagagcatctctgcccaacagggactcagacttagctcaacccgtcagtacccagactgaccactgcctctgcctcttcttctccagg

SEQ ID NO: 1352 - DNA, 120 bp, Homo sapiens
tgggaaggagtacctctaccgctctctggctgtgtgaccttggtactgccattttccttcccttaaacagctttaattaatacctgccctgccaccagctccatataacatcatgaattt

SEQ ID NO: 1353 - DNA, 120 bp, Homo sapiens
tggttccttccagtacagcactgcaatgcgagggagagtgagcgtccccagctgccctctcccaaccctgccagcctggtagccaaaagctaagaataaccactaggcttttggcacaaa

SEQ ID NO: 1354 - DNA, 120 bp, Homo sapiens
cagcccaaactgctcccacacatttctgacacccactgaagaggcagtactctccagttgagtgcaactaatccctgccagccttcctaaggtgctaatggggagcctcagacccaaaga

SEQ ID NO: 1355 - DNA, 120 bp, Homo sapiens
tcctcctgtaggaagccactgtgttagagctctcagggtgtctacaaacatctagataagtgtttctcaacatggattctgttgacatattgggaaaaataattttgtcattatgtagaa

SEQ ID NO: 1356 - DNA, 120 bp, Homo sapiens
ctgctttgtggttttcagatctccgcaaagttgcctatgatgccatcttctggggcaggccttgaaaagccccctaactgttcatctcccatccttaaacccctgctgcccttaagcagt

SEQ ID NO: 1357 - DNA, 120 bp, Homo sapiens
cccaaccctgccagcctggtagccaaaagctaagaataaccactaggcttttggcacaaactgctttgtggttttcagatctccgcaaagttgcctatgatgccatcttctggggcaggc

SEQ ID NO: 1358 - DNA, 120 bp, Homo sapiens
cttgaaaagccccctaactgttcatctcccatccttaaacccctgctgcccttaagcagttgaatcaactccatgagcacctgctctaccttccccagagccctgagacctttggagctt

SEQ ID NO: 1359 - DNA, 120 bp, Homo sapiens
tcccaaggaccttgagctagtctcaccacagagaatccttccagtcaggacaggaattgaccttcccccctcttcagccctctaacccagaagagtcttaaaataaaatctacaggccaa

SEQ ID NO: 1360 - DNA, 120 bp, Homo sapiens
agtgcaactaatccctgccagccttcctaaggtgctaatggggagcctcagacccaaagagagagagaagaacttgtccaatgtaggtcaacccatttgctgatctcttcaacaccaagc

SEQ ID NO: 1361 - DNA, 120 bp, Homo sapiens
tgaaaagtgataattggttgttctctaaatcctcatttccttctctgcctctaagtaagcatgtggcatcccacctcggcttcctggtccagtcttgttcatcttataaaaaggcctccc

SEQ ID NO: 1362 - DNA, 120 bp, Homo sapiens
ccttcccccctcttcagccctctaacccagaagagtcttaaaataaaatctacaggccaatggttccttccagtacagcactgcaatgcgagggagagtgagcgtccccagctgccctct

SEQ ID NO: 1363 - DNA, 120 bp, Homo sapiens
tacggggtcagaggcctagacccatcaaacccagggctcctgaaacaataggacccctattcctcctgtaggaagccactgtgttagagctctcagggtgtctacaaacatctagataag

SEQ ID NO: 1364 - DNA, 120 bp, Homo sapiens
gagagagaagaacttgtccaatgtaggtcaacccatttgctgatctcttcaacaccaagctctattatcagccctgtttttttctttctttctctctttgtagagatcacatgttgtgag

SEQ ID NO: 1365 - DNA, 120 bp, Homo sapiens
atgtggcatcccacctcggcttcctggtccagtcttgttcatcttataaaaaggcctccctacggggtcagaggcctagacccatcaaacccagggctcctgaaacaataggacccctat

SEQ ID NO: 1366 - DNA, 120 bp, Homo sapiens
ctatgtctgctgtatgaggtccctcgatggcattgtgaatggagctggccagagaaatcttcccaaggaccttgagctagtctcaccacagagaatccttccagtcaggacaggaattga

SEQ ID NO: 1367 - DNA, 120 bp, Homo sapiens
tgaatcaactccatgagcacctgctctaccttccccagagccctgagacctttggagctttgaaaagtgataattggttgttctctaaatcctcatttccttctctgcctctaagtaagc

SEQ ID NO: 1368 - DNA, 120 bp, Homo sapiens
tatggttaacatacctggcaccagcctactctataccaaataggattccagtcattctgacagcccaaactgctcccacacatttctgacacccactgaagaggcagtactctccagttg

SEQ ID NO: 1369 - DNA, 120 bp, Homo sapiens
tgtttctcaacatggattctgttgacatattgggaaaaataattttgtcattatgtagaatatggttaacatacctggcaccagcctactctataccaaataggattccagtcattctga

SEQ ID NO: 1370 - DNA, 120 bp, Homo sapiens
cctgagttgaataattctacaccatctgccctcttctctctttctccaggacagccaagatctctctgagataggatgctgagcttccacccagacaataccaggcctgctcatcctatg

SEQ ID NO: 1371 - DNA, 120 bp, Homo sapiens
ctcagggtcagccaatcttactaaaaaattctctacagtgaaagagcttggagcaacactgttctgctcaattgatttgtgataccatctaaacacttcctctttctagttgggcttcag

SEQ ID NO: 1372 - DNA, 120 bp, Homo sapiens
tctctctgagataggatgctgagcttccacccagacaataccaggcctgctcatcctatggagtaggctagtggcttggaaaccaaaatgtcaaaccatagcctttaggctccatctggg

SEQ ID NO: 1373 - DNA, 120 bp, Homo sapiens
gagtaggctagtggcttggaaaccaaaatgtcaaaccatagcctttaggctccatctgggaggtctttgtcctcaccacttaagtgggtgtcaaatttccttccctttctgcacacgctg

SEQ ID NO: 1374 - DNA, 120 bp, Homo sapiens
ggcctgcagcaatgttaaaggaatcctcattccagcattgtgatttcaatggtaaaaagattgcagcattgtcatcaacagaggtgggaaagtacattggagactggagcagagccagac

SEQ ID NO: 1375 - DNA, 120 bp, Homo sapiens
gttctgctcaattgatttgtgataccatctaaacacttcctctttctagttgggcttcagcctgagttgaataattctacaccatctgccctcttctctctttctccaggacagccaaga

SEQ ID NO: 1376 - DNA, 120 bp, Homo sapiens
ttgcagcattgtcatcaacagaggtgggaaagtacattggagactggagcagagccagacctcagggtcagccaatcttactaaaaaattctctacagtgaaagagcttggagcaacact

SEQ ID NO: 1377 - DNA, 120 bp, Homo sapiens
tagaggtatgccatggtcagccatggaaccgagaggttgctcttccttgaaaagctggccaagcattggccacttccccatataatttataggtgataatgtggtgatctgttcagaagt

SEQ ID NO: 1378 - DNA, 120 bp, Homo sapiens
cgatttttgaagtgctgaaaactggaaggcctactagcatgaggatgctgtgtcttctcttagaggtatgccatggtcagccatggaaccgagaggttgctcttccttgaaaagctggcc

SEQ ID NO: 1379 - DNA, 120 bp, Homo sapiens
aagcattggccacttccccatataatttataggtgataatgtggtgatctgttcagaagtgactataataaatgcaactcacatatgtctacagtttccaaactgtggtaaggagcagcc

SEQ ID NO: 1380 - DNA, 120 bp, Homo sapiens
catttgcaaatcttgatgtcctgatgttaagagctgactactggggcttctcctaaaaatccttcatgttgagctgcctggaaggcaggttctcattctggctgtagctgagatgttaga

SEQ ID NO: 1381 - DNA, 120 bp, Homo sapiens
actgtagtcagggagaccatgtgcctcccccattgtgttcatttggttaggctttcctgtccctgactcagaaaacagaaggggcacagagacctggaaattccatgtgctaacccatat

SEQ ID NO: 1382 - DNA, 120 bp, Homo sapiens
ccctgactcagaaaacagaaggggcacagagacctggaaattccatgtgctaacccatatcctggccagagaagatgagtagttatcagggtgtcaggattttggaaaacagagagagaa

SEQ ID NO: 1383 - DNA, 120 bp, Homo sapiens
ccttcatgttgagctgcctggaaggcaggttctcattctggctgtagctgagatgttagaactgtagtcagggagaccatgtgcctcccccattgtgttcatttggttaggctttcctgt

SEQ ID NO: 1384 - DNA, 120 bp, Homo sapiens
aatttctagtattccaggccagaggcaaaggggtcaacaggatgaccaaacacttcgggtcatttgcaaatcttgatgtcctgatgttaagagctgactactggggcttctcctaaaaat

SEQ ID NO: 1385 - DNA, 120 bp, Homo sapiens
ccttcttcattccccctccccatccccactctactctctctcagcatcattttcctaacaagaaacaatttcatgactagaagccaatttatttgctagaagtcaacctccatcagattc

SEQ ID NO: 1386 - DNA, 120 bp, Homo sapiens
atctatgtaagcaactcagataggatttgtatggcagccaaggaacttttctttaatatcttttctaagagccctctcttagcccctacggagggagaagggcaaaatttgatattcaaa

SEQ ID NO: 1387 - DNA, 120 bp, Homo sapiens
ccctctggctttgagtgtggtccaggaagaaaatgtggtgaagaaaagaacacgggtcacagtgtcccagctggatattgtgaaaggggtggaggagttgagaacagagcagttgggact

SEQ ID NO: 1388 - DNA, 120 bp, Homo sapiens
cccacctatccccagtctgtctttgggacaaggcctttttgactggttacagcaggtctctgaatttttccatagcttctgctatagaaacagacatgggccaccttgtattctttgcag

SEQ ID NO: 1389 - DNA, 120 bp, Homo sapiens
agccaatgataatatgcttctctagagtctggcaccacctgttgggaggtgcttccattcccctctggctttgagtgtggtccaggaagaaaatgtggtgaagaaaagaacacgggtcac

SEQ ID NO: 1390 - DNA, 120 bp, Homo sapiens
ggccccaagcacacagacttcaactaacaggaagccaagtagatggttccctgtgggggtgggggtcaagtctgtggtcagaaaacttggtgctttgtctaatgctccttcgtgggcatg

SEQ ID NO: 1391 - DNA, 120 bp, Homo sapiens
aagagtttggatggctccaaatcaccccccaggaattcctgtgcatgaaagcactgctactcttcagcattagtaagtgcctagaagtgcagggaatgccccctgagggcacagagattc

SEQ ID NO: 1392 - DNA, 120 bp, Homo sapiens
ggtaccgcatgcacaagtcccggatgtacagccagtgtgtccgaatgaggcacctctctcaagagtttggatggctccaaatcaccccccaggaattcctgtgcatgaaagcactgctac

SEQ ID NO: 1393 - DNA, 120 bp, Homo sapiens
cttcccctccccattctgtcttcatcccacatcagttccagtggatgggctgaaaaatcaaaaattctttgatgaacttcgaatgaactacatcaaggaactcgatcgtatcattgcatg

SEQ ID NO: 1394 - DNA, 120 bp, Homo sapiens
gcaggagaaacagcaagctcttcttggaaaacctggcgagggatggcaatcagagacattccctctgggcttattgtaaacttcccctcattcctttttcctctgtgtatctccttccca

SEQ ID NO: 1395 - DNA, 120 bp, Homo sapiens
ctaaaaaatgagtatctgagaagagtagaaaaagaaaaggttcaggaaatttgatttacttgactcctttcagatcggatccagctatcctttcccctgagatctccctgacagactgaa

SEQ ID NO: 1396 - DNA, 120 bp, Homo sapiens
agaaacaatttcatgactagaagccaatttatttgctagaagtcaacctccatcagattccccacctatccccagtctgtctttgggacaaggcctttttgactggttacagcaggtctc

SEQ ID NO: 1397 - DNA, 120 bp, Homo sapiens
ctccgtgcagcctgtaagcaaacgatggagggtgctttatcagggagaacagcctgatagagccaatgataatatgcttctctagagtctggcaccacctgttgggaggtgcttccattc

SEQ ID NO: 1398 - DNA, 120 bp, Homo sapiens
agtgtcccagctggatattgtgaaaggggtggaggagttgagaacagagcagttgggactcagggaagggacttgcagcagatgaattctctaggcagacaaaacagacctggatgtttt

SEQ ID NO: 1399 - DNA, 120 bp, Homo sapiens
ttttctaagagccctctcttagcccctacggagggagaagggcaaaatttgatattcaaagctatgtgttttggttatctaaatcagggttttactgtgaatgacataaaagcttaggtc

SEQ ID NO: 1400 - DNA, 120 bp, Homo sapiens
ggcagtagagcaggaggcatttcctcctggaaagatttcctcttctgccaacaggaggagatctatgtaagcaactcagataggatttgtatggcagccaaggaacttttctttaatatc

SEQ ID NO: 1401 - DNA, 120 bp, Homo sapiens
tgaatttttccatagcttctgctatagaaacagacatgggccaccttgtattctttgcagggcagtagagcaggaggcatttcctcctggaaagatttcctcttctgccaacaggaggag

SEQ ID NO: 1402 - DNA, 120 bp, Homo sapiens
gctatgtgttttggttatctaaatcagggttttactgtgaatgacataaaagcttaggtcctaaaaaatgagtatctgagaagagtagaaaaagaaaaggttcaggaaatttgatttact

SEQ ID NO: 1403 - DNA, 120 bp, Homo sapiens
caaaagaaaaaatcccacatcctgctcaagacgcttctaccagctcaccaagctcctggactccgtgcagcctgtaagcaaacgatggagggtgctttatcagggagaacagcctgatag

SEQ ID NO: 1404 - DNA, 120 bp, Homo sapiens
aaaattctttgatgaacttcgaatgaactacatcaaggaactcgatcgtatcattgcatgcaaaagaaaaaatcccacatcctgctcaagacgcttctaccagctcaccaagctcctgga

SEQ ID NO: 1405 - DNA, 120 bp, Homo sapiens
tcttcagcattagtaagtgcctagaagtgcagggaatgccccctgagggcacagagattcagagaggaccacttttgccattaaaacattattagggaaaagccagctcctggacatttc

SEQ ID NO: 1406 - DNA, 120 bp, Homo sapiens
gggggtcaagtctgtggtcagaaaacttggtgctttgtctaatgctccttcgtgggcatgcttcccctccccattctgtcttcatcccacatcagttccagtggatgggctgaaaaatca

SEQ ID NO: 1407 - DNA, 120 bp, Homo sapiens
agagaggaccacttttgccattaaaacattattagggaaaagccagctcctggacatttcccttcttcattccccctccccatccccactctactctctctcagcatcattttcctaaca

SEQ ID NO: 1408 - DNA, 120 bp, Homo sapiens
ccctctgggcttattgtaaacttcccctcattcctttttcctctgtgtatctccttcccaggtaccgcatgcacaagtcccggatgtacagccagtgtgtccgaatgaggcacctctctc

SEQ ID NO: 1409 - DNA, 120 bp, Homo sapiens
tgactcctttcagatcggatccagctatcctttcccctgagatctccctgacagactgaaggccccaagcacacagacttcaactaacaggaagccaagtagatggttccctgtgggggt

SEQ ID NO: 1410 - DNA, 120 bp, Homo sapiens
gagagatggagtgcggaggcttgggtgagagcacaagctggagaagtcttgagtcagagagcttacaatggtataagacatctcttgggagccctcagtgactccatggagaccatttct

SEQ ID NO: 1411 - DNA, 120 bp, Homo sapiens
gaccaaaaatcagaggttggggaagaggctagcagaggccacctccttgtcaaccctgtttttctccctcttattgttccctacagattgcgagagagctgcatcagttcacttttgacc

SEQ ID NO: 1412 - DNA, 120 bp, Homo sapiens
ctcatatggcccagtgtcaagttgtgcttgtttacagcactactctgtgccagccacacaaacgtttacttatcttatgccacgggaagtttagagagctaagattatctggggaaatca

SEQ ID NO: 1413 - DNA, 120 bp, Homo sapiens
ccattgtggctcctatctgtgttttgaatggtgttgtatgcctttaaatctgtgatgatcctcatatggcccagtgtcaagttgtgcttgtttacagcactactctgtgccagccacaca

SEQ ID NO: 1414 - DNA, 120 bp, Homo sapiens
ctgttataactctgcactactcctctgcagtgccttggggaatttcctctattgatgtacagtctgtcatgaacatgttcctgaattctatttgctgggctttttttttctctttctctc

SEQ ID NO: 1415 - DNA, 120 bp, Homo sapiens
ctgtgcaagtgcccaagatcctttctgggaaagtcaagcccatctatttccacacccagtgaagcattggaaaccctatttccccaccccagctcatgccccctttcagatgtcttctgc

SEQ ID NO: 1416 - DNA, 120 bp, Homo sapiens
gaagcattggaaaccctatttccccaccccagctcatgccccctttcagatgtcttctgcctgttataactctgcactactcctctgcagtgccttggggaatttcctctattgatgtac

SEQ ID NO: 1417 - DNA, 120 bp, Homo sapiens
ctttctttttcttcttccctccctatctaaccctcccatggcaccttcagactttgcttcccattgtggctcctatctgtgttttgaatggtgttgtatgcctttaaatctgtgatgatc

SEQ ID NO: 1418 - DNA, 120 bp, Homo sapiens
agtctgtcatgaacatgttcctgaattctatttgctgggctttttttttctctttctctcctttctttttcttcttccctccctatctaaccctcccatggcaccttcagactttgcttc

SEQ ID NO: 1419 - DNA, 120 bp, Homo sapiens
cgacctcatgggggaggaccaaggaagtacggggaagggggaggaaacaaaaggctgaaagaccaaaaatcagaggttggggaagaggctagcagaggccacctccttgtcaaccctgtt

SEQ ID NO: 1420 - DNA, 120 bp, Homo sapiens
tttctccctcttattgttccctacagattgcgagagagctgcatcagttcacttttgacctgctaatcaagtcacacatggtgagcgtggactttccggaaatgatggcagagatcatct

SEQ ID NO: 1421 - DNA, 120 bp, Homo sapiens
tgctaatcaagtcacacatggtgagcgtggactttccggaaatgatggcagagatcatctctgtgcaagtgcccaagatcctttctgggaaagtcaagcccatctatttccacacccagt

SEQ ID NO: 1422 - DNA, 23 bp, artificial
  synthetic oligonucleotide primer
cgtaagaagaagaaaatgccaga

SEQ ID NO: 1423 - DNA, 19 bp, artificial
  synthetic oligonucleotide primer
tcaaactcggcattcatgg

SEQ ID NO: 1424 - DNA, 18 bp, artificial
  synthetic oligonucleotide primer
gtgggaaggcagctgaac

SEQ ID NO: 1425 - DNA, 18 bp, artificial
  synthetic oligonucleotide primer
cacctcagccaggacctc

SEQ ID NO: 1426 - DNA, 20 bp, artificial
  synthetic oligonucleotide primer
ggtactgaagtccgggaacc

SEQ ID NO: 1427 - DNA, 21 bp, artificial
  synthetic oligonucleotide primer
gaagatccttaaggggtgcaa

SEQ ID NO: 1428 - DNA, 20 bp, artificial
  synthetic oligonucleotide primer
tggtctgccctgtatgatgt

SEQ ID NO: 1429 - DNA, 20 bp, artificial
  synthetic oligonucleotide primer
aaaagctgtggggtttttcc

SEQ ID NO: 1430 - DNA, 20 bp, artificial
  synthetic oligonucleotide primer
tctatggcgctgagattgtg

SEQ ID NO: 1431 - DNA, 20 bp, artificial
  synthetic oligonucleotide primer
cttaatgtgcccgtccttgt

SEQ ID NO: 1432 - DNA, 70 bp, Homo sapiens
gagaacagagtcaggtattataatcagattagactctagggaggttagccagccatattgctgatatgtg

SEQ ID NO: 1433 - DNA, 70 bp, Homo sapiens
gagaacagagtcaggtattataatcagattagactcttgggagccctcagtgactccatggagaccattt

SEQ ID NO: 1434 - DNA, 70 bp, Homo sapiens
ttgagtcagagagcttacaatggtataagacatctcttgggagccctcagtgactccatggagaccattt

SEQ ID NO: 1435 - DNA, 69 bp, Homo sapiens
aagcccatatgatcttttccgaaacagacccttatctttacctccttctttggagtctttctcctactt

SEQ ID NO: 1436 - DNA, 69 bp, Homo sapiens
aagcccatatgatcttttccgaaacagacccttatctgttgtacttgggacttgaataaaggctgatat

SEQ ID NO: 1437 - DNA, 69 bp, Homo sapiens
tgggtttgcaaatcagtagaattttttattgctttctgttgtacttgggacttgaataaaggctgatat

SEQ ID NO: 1438 - DNA, 130 bp, Homo sapiens
tttctttggaaactttttaccttttcattgaagcccatatgatcttttccgaaacagacccttatctgttgtacttgggacttgaataaaggctgatatttgtgtcttgctggtaaagtgcttgtaaagt

SEQ ID NO: 1439 - DNA, 22 bp, artificial
  synthetic oligonucleotide primer
tagggttgcagctactctttcc

SEQ ID NO: 1440 - DNA, 22 bp, artificial
  synthetic oligonucleotide primer
tgcttagcactcaaacccagta

SEQ ID NO: 1441 - DNA, 23 bp, artificial
  synthetic oligonucleotide primer
tgtgtgttagagagagacagcga

SEQ ID NO: 1442 - DNA, 24 bp, artificial
  synthetic oligonucleotide primer
atttggagtgggtgagtagactgg

SEQ ID NO: 1443 - DNA, 23 bp, artificial
  synthetic oligonucleotide primer
tgcttttatcagggagaacagcc

SEQ ID NO: 1444 - DNA, 20 bp, artificial
  synthetic oligonucleotide primer
agctctctgactcagacttc

SEQ ID NO: 1445 - DNA, 20 bp, artificial
  synthetic oligonucleotide primer
gtgaatgtgaaggcacatgg

SEQ ID NO: 1446 - DNA, 20 bp, artificial
  synthetic oligonucleotide primer
ccacttgccttgcctagaag

SEQ ID NO: 1447 - DNA, 20 bp, artificial
  synthetic oligonucleotide primer
gggtggaggagttgagaaca

SEQ ID NO: 1448 - DNA, 20 bp, artificial
  synthetic oligonucleotide primer
caaagaaaggccagtttgga

SEQ ID NO: 1449 - DNA, 20 bp, artificial
  synthetic oligonucleotide primer
tggatggatagctactccgg

SEQ ID NO: 1450 - DNA, 20 bp, artificial
  synthetic oligonucleotide primer
tttaccctgctgagctgtcc

SEQ ID NO: 1451 - DNA, 22 bp, artificial
  synthetic oligonucleotide primer
gcagcaaagatttccaaactgg

SEQ ID NO: 1452 - DNA, 20 bp, artificial
  synthetic oligonucleotide primer
tgggtgtggaaatagatggg

SEQ ID NO: 1453 - DNA, 19 bp, artificial
  synthetic oligonucleotide primer
gaaggtgaaggtcggagtc

SEQ ID NO: 1454 - DNA, 20 bp, artificial
  synthetic oligonucleotide primer
gaagatggtgatgggatttc

SEQ ID NO: 1455 - DNA, 20 bp, artificial
  synthetic oligonucleotide primer
aggagggaagagtcccagtg

SEQ ID NO: 1456 - DNA, 20 bp, artificial
  synthetic oligonucleotide primer
tgggaagctactggttttgc

SEQ ID NO: 1457 - DNA, 18 bp, artificial
  synthetic oligonucleotide primer
ctgaagcgtggcctgaag

SEQ ID NO: 1458 - DNA, 20 bp, artificial
  synthetic oligonucleotide primer
cttcctcacctccactgagc

SEQ ID NO: 1459 - DNA, 20 bp, artificial
  synthetic oligonucleotide primer
ttttgcttgcccatagatcc

SEQ ID NO: 1460 - DNA, 20 bp, artificial
  synthetic oligonucleotide primer
actgggactggaggaggaat

SEQ ID NO: 1461 - DNA, 20 bp, artificial
  synthetic oligonucleotide primer
atcactgcacctgctcctct

SEQ ID NO: 1462 - DNA, 35 bp, artificial
  inversion point of AR exons 5-7 in LuCaP 136 xenograft
catggaccccaaggtgaaacatccaggtctgtttt

SEQ ID NO: 1463 - DNA, 35 bp, artificial
  inversion point of AR exons 5-7 in LuCaP 136 xenograft
gttctggagaaacagagagagagagagagagagat